(19) United States
(12) Patent Application Publication
(10) Pub. No.: US 2023/0374114 A1
(43) Pub. Date: Nov. 23, 2023

(54) CORONAVIRUS ANTIBODIES AND METHODS OF USE THEREOF

(71) Applicant: Dana-Farber Cancer Institute, Inc., Boston, MA (US)

(72) Inventors: Wayne A. Marasco, Wellsley, MA (US); Matthew Chang, Brookline, MA (US)

(21) Appl. No.: 17/919,486

(22) PCT Filed: Apr. 16, 2021
(86) PCT No.: PCT/US2021/027757

§ 371 Date: Oct. 17, 2022

Related U.S. Application Data
(60) Provisional application No. 63/011,063, filed on Apr. 16, 2020.
(60) Provisional application No. 63/016,154, filed on Apr. 27, 2020.
(60) Provisional application No. 63/021,672, filed on May 7, 2020.

(51) Int. Cl. C07K 16/10; A61P 31/14

(52) U.S. Cl. CPC C07K 16/1002; A61P 31/14; C07K 2317/565; C07K 2317/622

(57) ABSTRACT

The present invention provides monoclonal antibodies that neutralize SARS-CoV2 and methods of use thereof. The antibodies described herein can be used to treat SARS-CoV2 infections and symptoms thereof.

This application is an International Application which claims priority from U.S. Provisional Patent Application Nos. 63/011,063 filed on Apr. 16, 2020, 63/016,154 filed on Apr. 27, 2020, and 63/021,672 filed on May 7, 2020, the contents of which is incorporated herein by reference in its entirety.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### SEQUENCE LISTING

The instant application contains a Sequence Listing which has been submitted electronically in ASCII format and is hereby incorporated by reference in its entirety. Said ASCII copy, created on Jun. 7, 2021, is named 5031461-102WO1_SL.txt and is 1,255,191 bytes in size.

### FIELD OF THE INVENTION

This invention relates generally to severe acute respiratory syndrome-associated coronavirus 2 (SARS-CoV2) neutralizing antibodies as well as to methods for use thereof.

### BACKGROUND

Human monoclonal antibody (mAb) therapy offers considerable advantages for prophylaxis, preemptive and acute treatment in viral outbreak settings.

### SUMMARY

An aspect of the invention is directed to isolated monoclonal antibodies directed to Severe Acute Respiratory Syndrome coronavirus (SARS-CoV2). In some embodiments, the antibody binds to an epitope in SEQ ID NO: 979. In some embodiments, the antibody binds to an epitope in the receptor binding domain (RBD) of the spike protein (S). In some embodiments, the antibody neutralizes SARS-CoV2. In some embodiments the epitope is linear. In other embodiments, the epitope is non-linear. In some embodiments, the epitope comprises a region within amino acids 319-490 of SEQ ID NO: 980 of the spike protein. In other embodiments, the epitope comprises a region within amino acids 319-541 SEQ ID NO: 980 of the spike protein. In further embodiments, the monoclonal antibody inhibits viral and cell membrane fusion. In yet other embodiments, the monoclonal antibody competes with the binding of a monoclonal antibody to the spike protein. In some embodiments, the monoclonal antibody blocks the binding of SARS-CoV2 spike protein to angiotensin converting enzyme 2 (ACE2) cell surface receptor. In another embodiment, the monoclonal antibody is a fully human antibody. In some embodiments, the monoclonal antibody comprises: (a) a heavy chain with three CDRs comprising the amino acid sequences GGSIRTHS (SEQ ID NO:93), IHHSGAT (SEQ ID NO:94), and ARGPGILSY (SEQ ID NO:95) respectively, and/or a light chain with three CDRs comprising the amino acid sequences SSNIGSNT (SEQ ID NO:227), SNN (SEQ ID NO:228), and AAWDDSLNVHYV (SEQ ID NO:229) respectively; (b) a heavy chain with three CDRs comprising the amino acid sequences GGSISSYY (SEQ ID NO:96), IYTSGST (SEQ ID NO:97), and ARDVGFGWFDR (SEQ ID NO:98) respectively and/or a light chain with three CDRs comprising the amino acid sequences SGSIASNY (SEQ ID NO:230), EDN (SEQ ID NO:231), and QSFDSASLWV (SEQ ID NO:232) respectively; (c) a heavy chain with three CDRs comprising the amino acid sequences GGSIRTHS (SEQ ID NO:99), IHHSGAT (SEQ ID NO:100), and ARGPGILSY (SEQ ID NO:101) respectively and/or a light chain with three CDRs comprising the amino acid sequences SSNIGSND (SEQ ID NO:233), SNN (SEQ ID NO:234), and ATWDDSLSAGV (SEQ ID NO:235) respectively; (d) a heavy chain with three CDRs comprising the amino acid sequences GDSVSSYSDA (SEQ ID NO:102), TYYRSKWYN (SEQ ID NO:103), and AREIVATTPFRNYYYGMDV (SEQ ID NO:104) respectively and/or a light chain with three CDRs comprising the amino acid sequences SGSIASNY (SEQ ID NO:236), QDK (SEQ ID NO:237), and QSYDSSSLWV (SEQ ID NO:238) respectively; (e) a heavy chain with three CDRs comprising the amino acid sequences GFTFSHYD (SEQ ID NO:105), IGYDGTNL (SEQ ID NO: 106), and ARAANYYDSSGYGRADAFDI (SEQ ID NO:107) respectively and/or a light chain with three CDRs comprising the amino acid sequences TGSIAGNY (SEQ ID NO:239), DDN (SEQ ID NO:240), and QSYDSGNRGV (SEQ ID NO:241) respectively; (f) a heavy chain with three CDRs comprising the amino acid sequences GFTFSDFP (SEQ ID NO:108), ISYDGNIK (SEQ ID NO:109), and AARGGSSFDI (SEQ ID NO:2780) respectively and/or a light chain with three CDRs comprising the amino acid sequences TSNIGNNA (SEQ ID NO:242), YNE (SEQ ID NO:243), and AAWDDSLSGHVV (SEQ ID NO:244) respectively; (g) a heavy chain with three CDRs comprising the amino acid sequences GFSLSTTGVG (SEQ ID NO:111), IYWNDDK (SEQ ID NO:112), and ARISGSGYFYPFDI (SEQ ID NO:113) respectively and/or a light chain with three CDRs comprising the amino acid sequences SGSIASNY (SEQ ID NO:245), EDN (SEQ ID NO:246), and QSYDSSSLWV (SEQ ID NO:247) respectively; (h) a heavy chain with three CDRs comprising the amino acid sequences GYTFSDYY (SEQ ID NO: 120), IDPNSGGT (SEQ ID NO:121), and ARDRGRGGQAGAFDY (SEQ ID NO:978) respectively and/or a light chain with three CDRs comprising the amino acid sequences KIGSKS (SEQ ID NO:254), DDS (SEQ ID NO:255), and HVWDSSSDQNV (SEQ ID NO:256) respectively; (i) a heavy chain with three CDRs comprising the amino acid sequences GFTFSSYA (SEQ ID NO:122), ISYGGSNK (SEQ ID NO:123), and AKVRGSGWYWGSAFDI (SEQ ID NO:124) respectively and/or a light chain with three CDRs comprising the amino acid sequences SLRAYF (SEQ ID NO:257), GQD (SEQ ID NO:258), and NSRDSGENHLI (SEQ ID NO:259) respectively; (j) a heavy chain with three CDRs comprising the amino acid sequences GYSFTGSH (SEQ ID NO:125), INPDSGVI (SEQ ID NO:126), and ARDKAIGYVWALDY (SEQ ID NO:127) respectively and/or a light chain with three CDRs comprising the amino acid sequences SSDVGTYNR (SEQ ID NO:260), EVS (SEQ ID NO:261), and SSYTRTFTYV (SEQ ID NO:262) respectively; (k) a heavy chain with three CDRs comprising the amino acid sequences GVSLDTIGMR (SEQ ID NO:128), IDWDDDK (SEQ ID NO: 129), and ARSGLLYDLDV (SEQ ID NO:130) respectively and/or a light chain with three CDRs comprising the amino acid sequences DSDIGANF (SEQ ID NO:263), RNT (SEQ ID NO:264), and QSYDSSLSAYV (SEQ ID NO:265) respectively; (1) a heavy chain with three CDRs comprising the amino acid sequences GYSFTSYW (SEQ ID NO:134), IYPGDSDT (SEQ ID NO:135), and ARGWQWHDY (SEQ ID NO:136) respectively and/or a light chain with three CDRs comprising the amino acid sequences SLRSYY (SEQ ID NO:269), DKD (SEQ ID NO:270), and NSRDRSDNHVV (SEQ ID NO:271) respectively; (m) a heavy chain with three CDRs comprising the amino acid sequences GDSVSSRSSA (SEQ ID NO:137), TYYRSNWNY (SEQ ID NO:138), and VRNMRPDFDL (SEQ ID NO:139) respectively and/or a light chain with three CDRs comprising the amino acid sequences QSVSNN (SEQ ID NO:272), DAT (SEQ ID NO:273), and QQYDNLPV (SEQ ID NO:274) respectively; (n) a heavy chain with three CDRs comprising the amino acid sequences GYTFTTSG (SEQ ID NO:140), ISAYNGNT (SEQ ID NO:141), and ARDFHLYYGMDV (SEQ ID NO:142) respectively and/or a light chain with three CDRs comprising the amino acid sequences SSDVGAYNY (SEQ ID NO:275), DVT (SEQ ID NO:276), and AVWDDGLNGRVV (SEQ ID NO:277) respectively; (o) a heavy chain with three CDRs comprising the amino acid sequences GGTFSSYA (SEQ ID NO:143), INPNSGGT (SEQ ID NO:144), and ARGSGGYYLG (SEQ ID NO:145) respectively and/or a light chain with three CDRs comprising the amino acid sequences SNNVGNQG (SEQ ID NO:278), MNN (SEQ ID NO:279), and SAWDSSLSRWV (SEQ ID NO:280) respectively; (p) a heavy chain with three CDRs comprising the amino acid sequences GGTFSSYT (SEQ ID NO: 146), IIPILGTP (SEQ ID NO: 147), and AVGSGWYSGFDY (SEQ ID NO:148) respectively and/or a light chain with three CDRs comprising the amino acid sequences SGSIASNY (SEQ ID NO:281), EDS (SEQ ID NO:282), and QSFHNSNPVI (SEQ ID NO:283) respectively; (q) a heavy chain with three CDRs comprising the amino acid sequences GFTFSSYW (SEQ ID NO:149), IKQDGSEK (SEQ ID NO:150), and ARGFYYYGAFDI (SEQ ID NO:151) respectively and/or a light chain with three CDRs comprising the amino acid sequences SGSIASNY (SEQ ID NO:284), EDN (SEQ ID NO:285), and QSYDSSNHWV (SEQ ID NO:286) respectively; (r) a heavy chain with three CDRs comprising the amino acid sequences GFTFDDYA (SEQ ID NO:152), IDWNSGVI (SEQ ID NO:153), and AKDAYSYGFLGAFDI (SEQ ID NO:154) respectively and/or a light chain with three CDRs comprising the amino acid sequences NIGSKS (SEQ ID NO:287), EDR (SEQ ID NO:288), and QVWDGDSDHYV (SEQ ID NO:289) respectively; (s) a heavy chain with three CDRs comprising the amino acid sequences GFTFDDYA (SEQ ID NO:155), IDWNSGVI (SEQ ID NO:156), and ARDILPSNFDGKKIIVFQPPAKRDLDNYYGMDV (SEQ ID NO:157) respectively and/or a light chain with three CDRs comprising the amino acid sequences SSDVGGYNL (SEQ ID NO:290), EGS (SEQ ID NO:291), and SSYTITDVVV (SEQ ID NO:292) respectively; or (t) a heavy chain with three CDRs comprising the amino acid sequences GYSFTSNW (SEQ ID NO:158), IFPGDSDT (SEQ ID NO:159), and ARESYNAYGS (SEQ ID NO:160) respectively and/or a light chain with three CDRs comprising the amino acid sequences SSNIGSNP (SEQ ID NO:293), SNN (SEQ ID NO:294), and AAWDDSLSGVV (SEQ ID NO:295) respectively.

In other embodiments, the monoclonal antibody comprises: (a) a heavy chain with three CDRs comprising the amino acid sequences GFTFTTYG (SEQ ID NO:114), ISYDGSIK (SEQ ID NO:115), and ARVGDSSSYYGIDA (SEQ ID NO:116) respectively, and/or a light chain with three CDRs comprising the amino acid sequences SSNIGSNS (SEQ ID NO:248), SNN (SEQ ID NO:249), and AAWDDSLTGYV (SEQ ID NO:250) respectively; (b) a heavy chain with three CDRs comprising the amino acid sequences GFTFSSHA (SEQ ID NO:117), ISYDGSYT (SEQ ID NO:118), and ARDWVNFGMDV (SEQ ID NO:119) respectively, and/or a light chain with three CDRs comprising the amino acid sequences SSDVGGYNY (SEQ ID NO:251), EVS (SEQ ID NO:252), and AAWDDSLSGPV (SEQ ID NO:253) respectively; or (c) a heavy chain with three CDRs comprising the amino acid sequences GFTFSDYP (SEQ ID NO:131), TSYDGRIK (SEQ ID NO:132), and ARDPGWLRSVGMDV (SEQ ID NO:133) respectively, and/or a light chain with three CDRs comprising the amino acid sequences SGSIARNY (SEQ ID NO:266), ADR (SEQ ID NO:267), and QSYDSSNQAAV (SEQ ID NO:268) respectively. In yet further embodiments, the monoclonal antibody comprises:
a) a heavy chain with three CDRs comprising the amino acid sequences GYTFTSYG (SEQ ID NO:161), ISAYNGNT (SEQ ID NO:162), and ARGFPQLGSDY (SEQ ID NO: 163) respectively, and/or a light chain with three CDRs comprising the amino acid sequences SGSIASNY (SEQ ID NO:296), EDN (SEQ ID NO:297), and QAWDSNSYV (SEQ ID NO:2781) respectively;
b) a heavy chain with three CDRs comprising the amino acid sequences GGTFSSYA (SEQ ID NO:164), ISGYNGNT (SEQ ID NO:165), and ARQMKDSGNYWEYYYYGMDV (SEQ ID NO:166) respectively, and/or a light chain with three CDRs comprising the amino acid sequences NIGSES (SEQ ID NO:299), EDR (SEQ ID NO:300), and QVWNPSGSLQYV (SEQ ID NO:301) respectively;
c) a heavy chain with three CDRs comprising the amino acid sequences GYTFTSYG (SEQ ID NO:167), ISTYNGNT (SEQ ID NO:168), and ARDVFGHFDY (SEQ ID NO:169) respectively, and/or a light chain with three CDRs comprising the amino acid sequences SGNIATNY (SEQ ID NO:302), EDN (SEQ ID NO:303), and KSYDDGNHV (SEQ ID NO:304) respectively;
d) a heavy chain with three CDRs comprising the amino acid sequences GFSLTTTGVS (SEQ ID NO:170), IHWDDDK (SEQ ID NO:171), and ASFIMTVYAEYFED (SEQ ID NO: 172) respectively, and/or a light chain with three CDRs comprising the amino acid sequences QSVSSN (SEQ ID NO:305), DVS (SEQ ID NO:306), and QQRGVWPLT (SEQ ID NO:307) respectively;
e) a heavy chain with three CDRs comprising the amino acid sequences GFSLSTSAMC (SEQ ID NO:173), IDWDNDR (SEQ ID NO:174), and AHSPYDSIWGSFRPSVYYFDY (SEQ ID NO:175) respectively, and/or a light chain with three CDRs comprising the amino acid sequences SGSIVSSY (SEQ ID NO:308), EHN (SEQ ID NO:309), and QSYDSQNGV (SEQ ID NO:310) respectively;
f) a heavy chain with three CDRs comprising the amino acid sequences GFTFSDYY (SEQ ID NO:176), ISSSSSDT (SEQ ID NO:177), and AMPTREPAY (SEQ ID NO:178) respectively, and/or a light chain with three CDRs comprising the amino acid sequences SSDLGTYNY (SEQ ID NO:311), DVF (SEQ ID NO:312), and SSYTSSSTYV (SEQ ID NO:313) respectively;
g) a heavy chain with three CDRs comprising the amino acid sequences GFAFSDFP (SEQ ID NO: 179), ISYDGSLK (SEQ ID NO:180), and AREGVSNSRPFDH (SEQ ID NO:181) respectively, and/or a light chain with three CDRs comprising the amino acid sequences SIGTKS (SEQ ID NO: 314), DDS (SEQ ID NO: 2782), and QVWESDDDDLV (SEQ ID NO:316) respectively;
h) a heavy chain with three CDRs comprising the amino acid sequences GFTFSSYA (SEQ ID NO:182), ISSNGGST (SEQ ID NO:183), and TRDLWSGSADSFDI (SEQ ID NO:184) respectively, and/or a light chain with three CDRs comprising the amino acid sequences SLRRYY (SEQ ID NO:317), GKN (SEQ ID NO:318), and NSRDISDNQWQWI (SEQ ID NO:319) respectively;
i) a heavy chain with three CDRs comprising the amino acid sequences GFPFNAYY (SEQ ID NO:185), INQDGSEK (SEQ ID NO:186), and ARLYWWGMDV (SEQ ID NO:187) respectively, and/or a light chain with three CDRs comprising the amino acid sequences SSDVGGYKY (SEQ ID NO:320), DVN (SEQ ID NO:321), and SSYTGRMNLYV (SEQ ID NO:322) respectively;
j) a heavy chain with three CDRs comprising the amino acid sequences GFTFDDYA (SEQ ID NO:188), IDWNSGVI (SEQ ID NO:189), and AKDAYSYGFLGAFDI (SEQ ID NO:190) respectively, and/or a light chain with three CDRs comprising the amino acid sequences NIRTKG (SEQ ID NO:323), YAS (SEQ ID NO:324), and QVWDSSSDLVV (SEQ ID NO:325) respectively;
k) a heavy chain with three CDRs comprising the amino acid sequences GFTFDDYA (SEQ ID NO:191), ISWNSGSI (SEQ ID NO:192), and ARDWWGSIDH (SEQ ID NO:193) respectively, and/or a light chain with three CDRs comprising the amino acid sequences SSDVGGYDY (SEQ ID NO:326), DVS (SEQ ID NO:327), and SSYTSSSPVV (SEQ ID NO:328) respectively;
l) a heavy chain with three CDRs comprising the amino acid sequences GGSISSSNW (SEQ ID NO:194), IYHSGST (SEQ ID NO:195), and ARRGGTYHRGAFDI (SEQ ID NO:196) respectively, and/or a light chain with three CDRs comprising the amino acid sequences SRDVGSYDL (SEQ ID NO:329), EGS (SEQ ID NO:330), and SSYTSSNSLV (SEQ ID NO:331) respectively;
m) a heavy chain with three CDRs comprising the amino acid sequences GASISNSF (SEQ ID NO:197), TSYSGNS (SEQ ID NO:198), and ARREWIKGHFDY (SEQ ID NO:199) respectively, and/or a light chain with three CDRs comprising the amino acid sequences GGSIASNY (SEQ ID NO:332), EDN (SEQ ID NO:333), and QSYDSSNPVV (SEQ ID NO:334) respectively;
n) a heavy chain with three CDRs comprising the amino acid sequences GGSFTTHS (SEQ ID NO:200), ILPGGAT (SEQ ID NO:201), and ARGPGILSY (SEQ ID NO:202) respectively, and/or a light chain with three CDRs comprising the amino acid sequences SSIGSND (SEQ ID NO:335), SNN (SEQ ID NO:336), and AWDDSLSAVV (SEQ ID NO:337) respectively;
o) a heavy chain with three CDRs comprising the amino acid sequences GGSFRTHS (SEQ ID NO:203), IHHSGAT (SEQ ID NO:204), and ARGPGILSY (SEQ ID NO:205) respectively, and/or a light chain with three CDRs comprising the amino acid sequences SSNIGSNT (SEQ ID NO:338), INN (SEQ ID NO:339), and AEWYDSLNVHYV (SEQ ID NO:340) respectively;
p) a heavy chain with three CDRs comprising the amino acid sequences GGSIRTHS (SEQ ID NO:206), IHHSGAT (SEQ ID NO:207), and ARGPGILSY (SEQ ID NO:208) respectively, and/or a light chain with three CDRs comprising the amino acid sequences SSNIGSNT (SEQ ID NO:341), INN (SEQ ID NO:342), and AECYDSLNDHYV (SEQ ID NO:343) respectively;
q) a heavy chain with three CDRs comprising the amino acid sequences GGSIRTHS (SEQ ID NO:209), IHHSGAT (SEQ ID NO:210), and GRGPGILSY (SEQ ID NO:211) respectively, and/or a light chain with three CDRs comprising the amino acid sequences SSNIGSNT (SEQ ID NO:344), SNN (SEQ ID NO:345), and AAWDDSLNVHYV (SEQ ID NO:346) respectively;
r) a heavy chain with three CDRs comprising the amino acid sequences GYSFTSYW (SEQ ID NO:212), IYPGDSDT (SEQ ID NO:213), and ARQGDGGGYDY (SEQ ID NO:214) respectively, and/or a light chain with three CDRs comprising the amino acid sequences SSNIGSNP (SEQ ID NO:347), NNN (SEQ ID NO:348), and AAWDDSLNGL (SEQ ID NO:349) respectively;
s) a heavy chain with three CDRs comprising the amino acid sequences RYSFSNYW (SEQ ID NO:215), IYPYDSDT (SEQ ID NO:216), and ARQGSSQSFDI (SEQ ID NO:217) respectively, and/or a light chain with three CDRs comprising the amino acid sequences SLRSYY (SEQ ID NO:350), QDS (SEQ ID NO: 2783), and QAWDSNSYV (SEQ ID NO: 2781) respectively;
t) a heavy chain with three CDRs comprising the amino acid sequences GYSFTSYW (SEQ ID NO:218), IYPGDSDT (SEQ ID NO:219), and ARRRGSAAAFDT (SEQ ID NO:220) respectively, and/or a light chain with three CDRs comprising the amino acid sequences SSNIGSNP (SEQ ID NO:353), DNN (SEQ ID NO:354), and EAWDDSLSGPV (SEQ ID NO:355) respectively;
u) a heavy chain with three CDRs comprising the amino acid sequences GYSFTSYW (SEQ ID NO:221), IYPGDSDT (SEQ ID NO:222), and ARTTYSYGSFDY (SEQ ID NO:223) respectively, and/or a light chain with three CDRs comprising the amino acid sequences SSNIGGNS (SEQ ID NO:356), RNN (SEQ ID NO:357), and AAWDDSLNGWV (SEQ ID NO:358) respectively; or
v) a heavy chain with three CDRs comprising the amino acid sequences GDSVTSNSAA (SEQ ID NO:224), TYYSSKWYN (SEQ ID NO:225), and ARGWLRLSFDP (SEQ ID NO:226) respectively, and/or a light chain with three CDRs comprising the amino acid sequences SGSIASNY (SEQ ID NO:359), EDN (SEQ ID NO:360), and QSYDPNNHGVV (SEQ ID NO:361) respectively.

In other embodiments, the monoclonal antibody comprises: a) a heavy chain with three CDRs comprising the amino acid sequences GFSLSTSGVG (SEQ ID NO:754), IYWDDDK (SEQ ID NO:755), and ARISGSGYFYPFDI (SEQ ID NO:756) respectively, and/or a light chain with three CDRs comprising the amino acid sequences SGSIASNY (SEQ ID NO:802), EDN (SEQ ID NO:803), and QSYDSSNLWV (SEQ ID NO:804) respectively; b) a heavy chain with three CDRs comprising the amino acid sequences GDSVSSNSAA (SEQ ID NO:757), TYYRSRWYN (SEQ ID NO:758), and AREIRGFDY (SEQ ID NO:759) respectively, and/or a light chain with three CDRs comprising the amino acid sequences SSDVGAYNF (SEQ ID NO:805), DFN (SEQ ID NO:806), and SSYAGSNNFDVV (SEQ ID NO:807) respectively; c) a heavy chain with three CDRs comprising the amino acid sequences GFTFGDYA (SEQ ID NO:760), IRSKAYGGTT (SEQ ID NO:761), and TTADDDMDV (SEQ ID NO:762) respectively, and/or a light chain with three CDRs comprising the amino acid sequences SGTIASNY (SEQ ID NO:808), EDN (SEQ ID NO:809), and QSYDTSNHYV (SEQ ID NO:810) respectively; d) a heavy chain with three CDRs comprising the amino acid sequences GFTFSNYG (SEQ ID NO:763), IWERGSKK (SEQ ID NO:764), and AREGISMTGAEYFQH (SEQ ID NO:765) respectively, and/or a light chain with three CDRs comprising the amino acid sequences SSNIGAGYD (SEQ ID NO:811), GTN (SEQ ID NO:812), and QSYDNSLTDPYV (SEQ ID NO:813) respectively; e) a heavy chain with three CDRs comprising the amino acid sequences GFTFDDYA (SEQ ID NO:766), IDWNSGVI (SEQ ID NO:767), and AKDIGPGGSGSYYAFDI (SEQ ID NO:768) respectively, and/or a light chain with three CDRs comprising the amino acid sequences SSDVGGSKY (SEQ ID NO:814), DVT (SEQ ID NO:815), and AAWDDSLNGVV (SEQ ID NO:816) respectively; f) a heavy chain with three CDRs comprising the amino acid sequences GFSFSRYG (SEQ ID NO:769), IRHDGSKK (SEQ ID NO:770), and AKDGRLEAALDD (SEQ ID NO:771) respectively, and/or a light chain with three CDRs comprising the amino acid sequences SGSIANNF (SEQ ID NO:817), EDN (SEQ ID NO:818), and QSYDSSNLV (SEQ ID NO:819) respectively; g) a heavy chain with three CDRs comprising the amino acid sequences GYSFTSYW (SEQ ID NO:772), IYPGDSDT (SEQ ID NO:773), and ARRGDLDAFDI (SEQ ID NO:774) respectively, and/or a light chain with three CDRs comprising the amino acid sequences SANIGSNA (SEQ ID NO:820), GNT (SEQ ID NO:821), and AAWDDSLNGYV (SEQ ID NO:822) respectively; h) a heavy chain with three CDRs comprising the amino acid sequences GYRLSDYY (SEQ ID NO:775), IKQDGSEK (SEQ ID NO:776), and ARVRGWSRGYFDY (SEQ ID NO:777) respectively, and/or a light chain with three CDRs comprising the amino acid sequences SGSIASNY (SEQ ID NO:823), EDN (SEQ ID NO:824), and QSYDSSNHWV (SEQ ID NO:825) respectively; i) a heavy chain with three CDRs comprising the amino acid sequences GFTFDDYA (SEQ ID NO:778), ISWNSGSI (SEQ ID NO:779), and ARDWWGSIDH (SEQ ID NO:780) respectively, and/or a light chain with three CDRs comprising the amino acid sequences SSDVGGYDY (SEQ ID NO:826), DVS (SEQ ID NO:827), and SSYTSSSPVV (SEQ ID NO:828) respectively; j) a heavy chain with three CDRs comprising the amino acid sequences GFTFSHYD (SEQ ID NO:781), IGYDGTNL (SEQ ID NO:782), and ARAANYYDSSGYGRADAF (SEQ ID NO:2784) respectively, and/or a light chain with three CDRs comprising the amino acid sequences TGSIAGNY (SEQ ID NO:829), DDN (SEQ ID NO:830), and QSYDSGNRGV (SEQ ID NO:831) respectively; k) a heavy chain with three CDRs comprising the amino acid sequences GGTFSTYG (SEQ ID NO:784), IIPSLGIP (SEQ ID NO:785), and ARENIDLATNDF (SEQ ID NO:786) respectively, and/or a light chain with three CDRs comprising the amino acid sequences SRDIGAYGY (SEQ ID NO:832), EVR (SEQ ID NO:833), and SSYTSSSTLDVV (SEQ ID NO:834) respectively; 1) a heavy chain with three CDRs comprising the amino acid sequences GGTFSSSG (SEQ ID NO:787), IIPMLGTP (SEQ ID NO:788), and ARDGGNYDY (SEQ ID NO:789) respectively, and/or a light chain with three CDRs comprising the amino acid sequences SSNIGRNA (SEQ ID NO:835), SNN (SEQ ID NO:836), and SAWDTSLSTWV (SEQ ID NO:837) respectively; m) a heavy chain with three CDRs comprising the amino acid sequences GFTFSSYW (SEQ ID NO:790), IKQDGSEK (SEQ ID NO:791), and ARGFYYYGAFDI (SEQ ID NO:792) respectively, and/or a light chain with three CDRs comprising the amino acid sequences SGSIASNY (SEQ ID NO:838), EDN (SEQ ID NO:839), and QSYDSSNHWV (SEQ ID NO:840) respectively; n) a heavy chain with three CDRs comprising the amino acid sequences GFTFDDYA (SEQ ID NO:793), IDWNSGVI (SEQ ID NO:794), and AKDAYSYGFLGAFDI (SEQ ID NO:795) respectively, and/or a light chain with three CDRs comprising the amino acid sequences NIRTKG (SEQ ID NO:841), YAS (SEQ ID NO:842), and QVWDSSSDLVV (SEQ ID NO:843) respectively; o) a heavy chain with three CDRs comprising the amino acid sequences GYSFTGSH (SEQ ID NO:796), INPDSGVI (SEQ ID NO:797), and ARDKAIGYVWALDY (SEQ ID NO:798) respectively, and/or a light chain with three CDRs comprising the amino acid sequences SSDVGTYNR (SEQ ID NO:844), EVS (SEQ ID NO:845), and SSYTRTFTYV (SEQ ID NO:846) respectively; or p) a heavy chain with three CDRs comprising the amino acid sequences GASISNSF (SEQ ID NO:799), TSYSGNS (SEQ ID NO:800), and ARREWIKGHFDY (SEQ ID NO:801) respectively, and/or a light chain with three CDRs comprising the amino acid sequences GGSIASNY (SEQ ID NO:847), EDN (SEQ ID NO:848), and QSYDSSNPVV (SEQ ID NO:849) respectively. In embodiments, the monoclonal antibody comprises a heavy chain with three CDRs comprising the amino acid sequences GFSLTTSGVS (SEQ ID NO:983), IHWDDDK (SEQ ID NO:984), and ASFIMTVYAEYFED (SEQ ID NO:985) respectively, and/or a light chain with three CDRs comprising the amino acid sequences QSVSSN (SEQ ID NO:986), DVS (SEQ ID NO:987), and QQRGAWPLT (SEQ ID NO:988) respectively.

In other embodiments, the monoclonal antibody comprises:
a. a V_{H} amino acid sequence having SEQ ID NO: 1, and a V_{L} amino acid sequence having SEQ ID NO: 2;
b. a V_{H} amino acid sequence having SEQ ID NO: 3, and a V_{L} amino acid sequence having SEQ ID NO: 4;
c. a V_{H} amino acid sequence having SEQ ID NO: 5, and a V_{L} amino acid sequence having SEQ ID NO: 6;
d. a V_{H} amino acid sequence having SEQ ID NO: 7, and a V_{L} amino acid sequence having SEQ ID NO: 8;
e. a V_{H} amino acid sequence having SEQ ID NO: 9, and a V_{L} amino acid sequence having SEQ ID NO: 10;
f. a V_{H} amino acid sequence having SEQ ID NO: 11, and a V_{L} amino acid sequence having SEQ ID NO: 12;
g. a V_{H} amino acid sequence having SEQ ID NO: 13, and a V_{L} amino acid sequence having SEQ ID NO: 14;
h. a V_{H} amino acid sequence having SEQ ID NO: 19, and a V_{L} amino acid sequence having SEQ ID NO: 20;
i. a V_{H} amino acid sequence having SEQ ID NO: 21, and a V_{L} amino acid sequence having SEQ ID NO: 22;
j. a V_{H} amino acid sequence having SEQ ID NO: 23, and a V_{L} amino acid sequence having SEQ ID NO: 24;
k. a V_{H} amino acid sequence having SEQ ID NO: 25, and a V_{L} amino acid sequence having SEQ ID NO: 26;
l. a V_{H} amino acid sequence having SEQ ID NO: 29, and a V_{L} amino acid sequence having SEQ ID NO: 30;
m. a V_{H} amino acid sequence having SEQ ID NO: 31, and a V_{L} amino acid sequence having SEQ ID NO: 32;
n. a V_{H} amino acid sequence having SEQ ID NO: 33, and a V_{L} amino acid sequence having SEQ ID NO: 34;
o. a V_{H} amino acid sequence having SEQ ID NO: 35, and a V_{L} amino acid sequence having SEQ ID NO: 36;
p. a V_{H} amino acid sequence having SEQ ID NO: 37, and a V_{L} amino acid sequence having SEQ ID NO: 38;
q. a V_{H} amino acid sequence having SEQ ID NO: 39, and a V_{L} amino acid sequence having SEQ ID NO: 40;
r. a V_{H} amino acid sequence having SEQ ID NO: 41, and a V_{L} amino acid sequence having SEQ ID NO: 42;
s. a V_{H} amino acid sequence having SEQ ID NO: 43, and a V_{L} amino acid sequence having SEQ ID NO: 44; or
t. a V_{H} amino acid sequence having SEQ ID NO: 47, and a V_{L} amino acid sequence having SEQ ID NO: 48.

In some embodiments, the antibody comprises: (a) a V_{H} amino acid sequence having SEQ ID NO: 15, and a V_{L} amino acid sequence having SEQ ID NO: 16; (b) a V_{H} amino acid sequence having SEQ ID NO: 17, and a V_{L} amino acid sequence having SEQ ID NO: 18; or (c) a V_{H} amino acid sequence having SEQ ID NO: 27, and a V_{L} amino acid sequence having SEQ ID NO: 28. In other embodiments, the monoclonal antibody comprises:
a. a V_{H} amino acid sequence having SEQ ID NO: 49, and a V_{L} amino acid sequence having SEQ ID NO: 50;
b. a V_{H} amino acid sequence having SEQ ID NO: 51, and a V_{L} amino acid sequence having SEQ ID NO: 52;
c. a V_{H} amino acid sequence having SEQ ID NO: 53, and a V_{L} amino acid sequence having SEQ ID NO: 54;
d. a V_{H} amino acid sequence having SEQ ID NO: 55, and a V_{L} amino acid sequence having SEQ ID NO: 56;
e. a V_{H} amino acid sequence having SEQ ID NO: 57, and a V_{L} amino acid sequence having SEQ ID NO: 58;
f. a V_{H} amino acid sequence having SEQ ID NO: 59, and a V_{L} amino acid sequence having SEQ ID NO: 60;
g. a V_{H} amino acid sequence having SEQ ID NO: 61, and a V_{L} amino acid sequence having SEQ ID NO: 62;
h. a V_{H} amino acid sequence having SEQ ID NO: 63, and a V_{L} amino acid sequence having SEQ ID NO: 64;
i. a V_{H} amino acid sequence having SEQ ID NO: 65, and a V_{L} amino acid sequence having SEQ ID NO: 66;
j. a V_{H} amino acid sequence having SEQ ID NO: 67, and a V_{L} amino acid sequence having SEQ ID NO: 68;
k. a V_{H} amino acid sequence having SEQ ID NO: 69, and a V_{L} amino acid sequence having SEQ ID NO: 70;
l. a V_{H} amino acid sequence having SEQ ID NO: 71, and a V_{L} amino acid sequence having SEQ ID NO: 72;
m. a V_{H} amino acid sequence having SEQ ID NO: 73, and a V_{L} amino acid sequence having SEQ ID NO: 74;
n. a V_{H} amino acid sequence having SEQ ID NO: 75, and a V_{L} amino acid sequence having SEQ ID NO: 76;
o. a V_{H} amino acid sequence having SEQ ID NO: 77, and a V_{L} amino acid sequence having SEQ ID NO: 78;
p. a V_{H} amino acid sequence having SEQ ID NO: 79, and a V_{L} amino acid sequence having SEQ ID NO: 80;
q. a V_{H} amino acid sequence having SEQ ID NO: 81, and a V_{L} amino acid sequence having SEQ ID NO: 82;
r. a V_{H} amino acid sequence having SEQ ID NO: 83, and a V_{L} amino acid sequence having SEQ ID NO: 84;
s. a V_{H} amino acid sequence having SEQ ID NO: 85, and a V_{L} amino acid sequence having SEQ ID NO: 86;
t. a V_{H} amino acid sequence having SEQ ID NO: 87, and a V_{L} amino acid sequence having SEQ ID NO: 88;
u. a V_{H} amino acid sequence having SEQ ID NO: 89, and a V_{L} amino acid sequence having SEQ ID NO: 90; or
v. a V_{H} amino acid sequence having SEQ ID NO: 91, and a V_{L} amino acid sequence having SEQ ID NO: 92.

In some embodiments, the antibody comprises: a) a VH amino acid sequence having SEQ ID NO: 722, and a VL amino acid sequence having SEQ ID NO: 723; b) a VH amino acid sequence having SEQ ID NO: 724, and a VL amino acid sequence having SEQ ID NO: 725; c) a VH amino acid sequence having SEQ ID NO: 726, and a VL amino acid sequence having SEQ ID NO: 727; d) a VH amino acid sequence having SEQ ID NO: 728, and a VL amino acid sequence having SEQ ID NO: 729; e) a VH amino acid sequence having SEQ ID NO: 730, and a VL amino acid sequence having SEQ ID NO: 731; f) a VH amino acid sequence having SEQ ID NO: 732, and a VL amino acid sequence having SEQ ID NO: 733; g) a VH amino acid sequence having SEQ ID NO: 734, and a VL amino acid sequence having SEQ ID NO: 735; h) a VH amino acid sequence having SEQ ID NO: 736, and a VL amino acid sequence having SEQ ID NO: 737; i) a VH amino acid sequence having SEQ ID NO: 738, and a VL amino acid sequence having SEQ ID NO: 739; j) a VH amino acid sequence having SEQ ID NO: 740, and a VL amino acid sequence having SEQ ID NO: 741; k) a VH amino acid sequence having SEQ ID NO: 742, and a VL amino acid sequence having SEQ ID NO: 743; 1) a VH amino acid sequence having SEQ ID NO: 744, and a VL amino acid sequence having SEQ ID NO: 745; m) a VH amino acid sequence having SEQ ID NO: 746, and a VL amino acid sequence having SEQ ID NO: 747; n) a VH amino acid sequence having SEQ ID NO: 748, and a VL amino acid sequence having SEQ ID NO: 749; o) a VH amino acid sequence having SEQ ID NO: 750, and a VL amino acid sequence having SEQ ID NO: 751; or p) a VH amino acid sequence having SEQ ID NO: 752, and a VL amino acid sequence having SEQ ID NO: 753. In embodiments, the monoclonal antibody comprises a VH amino acid sequence having SEQ ID NO: 981, and a VL amino acid sequence having SEQ ID NO: 982.

An aspect of the invention is directed to isolated scFv antibodies directed to Severe Acute Respiratory Syndrome coronavirus (SARS-CoV2). In some embodiments, the antibody binds to an epitope in SEQ ID NO: 979. In some embodiments, the scFv antibody binds to an epitope in the receptor binding domain (RBD) of the spike protein of SARS-CoV2. In other embodiments, the scFv antibody neutralizes SARS-CoV2. In some embodiments the epitope is linear. In other embodiments, the epitope is non-linear. In some embodiments, the epitope comprises a region within amino acids 319-490 of SEQ ID NO: 980 of the spike protein. In other embodiments, the epitope comprises a region within amino acids 319-541 SEQ ID NO: 980 of the spike protein. In further embodiments, the scFv antibody inhibits viral and cell membrane fusion. In yet other embodiments, the scFv antibody competes with the binding of a monoclonal antibody to the spike protein. In some embodiments, the scFv antibody blocks the binding of SARS-CoV2 spike protein to angiotensin converting enzyme 2 (ACE2) cell surface receptor. In another embodiment, the scFv antibody is a fully human antibody. In some embodiments, the scFv antibody comprises: (a) a heavy chain with three CDRs comprising the amino acid sequences GGSIRTHS (SEQ ID NO:93), IHHSGAT (SEQ ID NO:94), and ARGPGILSY (SEQ ID NO:95) respectively, and/or a light chain with three CDRs comprising the amino acid sequences SSNIGSNT (SEQ ID NO:227), SNN (SEQ ID NO:228), and AAWDDSLNVHYV (SEQ ID NO:229) respectively; (b) a heavy chain with three CDRs comprising the amino acid sequences GGSISSYY (SEQ ID NO:96), IYTSGST (SEQ ID NO:97), and ARDVGFGWFDR (SEQ ID NO:98) respectively and/or a light chain with three CDRs comprising the amino acid sequences SGSIASNY (SEQ ID NO:230), EDN (SEQ ID NO:231), and QSFDSASLWV (SEQ ID NO:232) respectively; (c) a heavy chain with three CDRs comprising the amino acid sequences GGSIRTHS (SEQ ID NO:99), IHHSGAT (SEQ ID NO:100), and ARGPGILSY (SEQ ID NO:101) respectively and/or a light chain with three CDRs comprising the amino acid sequences SSNIGSND (SEQ ID NO:233), SNN (SEQ ID NO:234), and ATWDDSLSAGV (SEQ ID NO:235) respectively; (d) a heavy chain with three CDRs comprising the amino acid sequences GDSVSSYSDA (SEQ ID NO:102), TYYRSKWYN (SEQ ID NO:103), and AREIVATTPFRNYYYGMDV (SEQ ID NO:104) respectively and/or a light chain with three CDRs comprising the amino acid sequences SGSIASNY (SEQ ID NO:236), QDK (SEQ ID NO:237), and QSYDSSSLWV (SEQ ID NO:238) respectively; (e) a heavy chain with three CDRs comprising the amino acid sequences GFTFSHYD (SEQ ID NO:105), IGYDGTNL (SEQ ID NO: 106), and ARAANYYDSSGYGRADAFDI (SEQ ID NO:107) respectively and/or a light chain with three CDRs comprising the amino acid sequences TGSIAGNY (SEQ ID NO:239), DDN (SEQ ID NO:240), and QSYDSGNRGV (SEQ ID NO:241) respectively; (f) a heavy chain with three CDRs comprising the amino acid sequences GFTFSDFP (SEQ ID NO:108), ISYDGNIK (SEQ ID NO:109), and AARGGSSFDI (SEQ ID NO:2780) respectively and/or a light chain with three CDRs comprising the amino acid sequences TSNIGNNA (SEQ ID NO:242), YNE (SEQ ID NO:243), and AAWDDSLSGHVV (SEQ ID NO:244) respectively; (g) a heavy chain with three CDRs comprising the amino acid sequences GFSLSTTGVG (SEQ ID NO:111), IYWNDDK (SEQ ID NO:112), and ARISGSGYFYPFDI (SEQ ID NO:113) respectively and/or a light chain with three CDRs comprising the amino acid sequences SGSIASNY (SEQ ID NO:245), EDN (SEQ ID NO:246), and QSYDSSSLWV (SEQ ID NO:247) respectively; (h) a heavy chain with three CDRs comprising the amino acid sequences GYTFSDYY (SEQ ID NO: 120), IDPNSGGT (SEQ ID NO:121), and ARDRGRGGQAGAFDY (SEQ ID NO:978) respectively and/or a light chain with three CDRs comprising the amino acid sequences KIGSKS (SEQ ID NO:254), DDS (SEQ ID NO:255), and HVWDSSSDQNV (SEQ ID NO:256) respectively; (i) a heavy chain with three CDRs comprising the amino acid sequences GFTFSSYA (SEQ ID NO:122), ISYGGSNK (SEQ ID NO:123), and AKVRGSGWYWGSAFDI (SEQ ID NO:124) respectively and/or a light chain with three CDRs comprising the amino acid sequences SLRAYF (SEQ ID NO:257), GQD (SEQ ID NO:258), and NSRDSGENHLI (SEQ ID NO:259) respectively; (j) a heavy chain with three CDRs comprising the amino acid sequences GYSFTGSH (SEQ ID NO:125), INPDSGVI (SEQ ID NO:126), and ARDKAIGYVWALDY (SEQ ID NO:127) respectively and/or a light chain with three CDRs comprising the amino acid sequences SSDVGTYNR (SEQ ID NO:260), EVS (SEQ ID NO:261), and SSYTRTFTYV (SEQ ID NO:262) respectively; (k) a heavy chain with three CDRs comprising the amino acid sequences GVSLDTIGMR (SEQ ID NO:128), IDWDDDK (SEQ ID NO: 129), and ARSGLLYDLDV (SEQ ID NO:130) respectively and/or a light chain with three CDRs comprising the amino acid sequences DSDIGANF (SEQ ID NO:263), RNT (SEQ ID NO:264), and QSYDSSLSAYV (SEQ ID NO:265) respectively; (1) a heavy chain with three CDRs comprising the amino acid sequences GYSFTSYW (SEQ ID NO:134), IYPGDSDT (SEQ ID NO:135), and ARGWQWHDY (SEQ ID NO:136) respectively and/or a light chain with three CDRs comprising the amino acid sequences SLRSYY (SEQ ID NO:269), DKD (SEQ ID NO:270), and NSRDRSDNHVV (SEQ ID NO:271) respectively; (m) a heavy chain with three CDRs comprising the amino acid sequences GDSVSSRSSA (SEQ ID NO:137), TYYRSNWNY (SEQ ID NO:138), and VRNMRPDFDL (SEQ ID NO:139) respectively and/or a light chain with three CDRs comprising the amino acid sequences QSVSNN (SEQ ID NO:272), DAT (SEQ ID NO:273), and QQYDNLPV (SEQ ID NO:274) respectively; (n) a heavy chain with three CDRs comprising the amino acid sequences GYTFTTSG (SEQ ID NO:140), ISAYNGNT (SEQ ID NO:141), and ARDFHLYYGMDV (SEQ ID NO:142) respectively and/or a light chain with three CDRs comprising the amino acid sequences SSDVGAYNY (SEQ ID NO:275), DVT (SEQ ID NO:276), and AVWDDGLNGRVV (SEQ ID NO:277) respectively; (o) a heavy chain with three CDRs comprising the amino acid sequences GGTFSSYA (SEQ ID NO:143), INPNSGGT (SEQ ID NO:144), and ARGSGGYYLG (SEQ ID NO:145) respectively and/or a light chain with three CDRs comprising the amino acid sequences SNNVGNQG (SEQ ID NO:278), MNN (SEQ ID NO:279), and SAWDSSLSRWV (SEQ ID NO:280) respectively; (p) a heavy chain with three CDRs comprising the amino acid sequences GGTFSSYT (SEQ ID NO: 146), IIPILGTP (SEQ ID NO: 147), and AVGSGWYSGFDY (SEQ ID NO:148) respectively and/or a light chain with three CDRs comprising the amino acid sequences SGSIASNY (SEQ ID NO:281), EDS (SEQ ID NO:282), and QSFHNSNPVI (SEQ ID NO:283) respectively; (q) a heavy chain with three CDRs comprising the amino acid sequences GFTFSSYW (SEQ ID NO:149), IKQDGSEK (SEQ ID NO:150), and ARGFYYYGAFDI (SEQ ID NO:151) respectively and/or a light chain with three CDRs comprising the amino acid sequences SGSIASNY (SEQ ID NO:284), EDN (SEQ ID NO:285), and QSYDSSNHWV (SEQ ID NO:286) respectively; (r) a heavy chain with three CDRs comprising the amino acid sequences GFTFDDYA (SEQ ID NO:152), IDWNSGVI (SEQ ID NO:153), and AKDAYSYGFLGAFDI (SEQ ID NO:154) respectively and/or a light chain with three CDRs comprising the amino acid sequences NIGSKS (SEQ ID NO:287), EDR (SEQ ID NO:288), and QVWDGDSDHYV (SEQ ID NO:289) respectively; (s) a heavy chain with three CDRs comprising the amino acid sequences GFTFDDYA (SEQ ID NO:155), IDWNSGVI (SEQ ID NO:156), and ARDILPSNFDGKKIIVFQPPAKRDLDNYYGMDV (SEQ ID NO:157) respectively and/or a light chain with three CDRs comprising the amino acid sequences SSDVGGYNL (SEQ ID NO:290), EGS (SEQ ID NO:291), and SSYTITDVVV (SEQ ID NO:292) respectively; or (t) a heavy chain with three CDRs comprising the amino acid sequences GYSFTSNW (SEQ ID NO:158), IFPGDSDT (SEQ ID NO:159), and ARESYNAYGS (SEQ ID NO:160) respectively and/or a light chain with three CDRs comprising the amino acid sequences SSNIGSNP (SEQ ID NO:293), SNN (SEQ ID NO:294), and AAWDDSLSGVV (SEQ ID NO:295) respectively.

In other embodiments, the scFv antibody comprises: (a) a heavy chain with three CDRs comprising the amino acid sequences GFTFTTYG (SEQ ID NO:114), ISYDGSIK (SEQ ID NO:115), and ARVGDSSSYYGIDA (SEQ ID NO:116) respectively, and/or a light chain with three CDRs comprising the amino acid sequences SSNIGSNS (SEQ ID NO:248), SNN (SEQ ID NO:249), and AAWDDSLTGYV (SEQ ID NO:250) respectively; (b) a heavy chain with three CDRs comprising the amino acid sequences GFTFSSHA (SEQ ID NO:117), ISYDGSYT (SEQ ID NO:118), and ARDWVNFGMDV (SEQ ID NO:119) respectively, and/or a light chain with three CDRs comprising the amino acid sequences SSDVGGYNY (SEQ ID NO:251), EVS (SEQ ID NO:252), and AAWDDSLSGPV (SEQ ID NO:253) respectively; or (c) a heavy chain with three CDRs comprising the amino acid sequences GFTFSDYP (SEQ ID NO:131), TSYDGRIK (SEQ ID NO:132), and ARDPGWLRSVGMDV (SEQ ID NO:133) respectively, and/or a light chain with three CDRs comprising the amino acid sequences SGSIARNY (SEQ ID NO:266), ADR (SEQ ID NO:267), and QSYDSSNQAAV (SEQ ID NO:268) respectively. In yet further embodiments, the scFv antibody comprises:
a) a heavy chain with three CDRs comprising the amino acid sequences GYTFTSYG (SEQ ID NO:161), ISAYNGNT (SEQ ID NO:162), and ARGFPQLGSDY (SEQ ID NO: 163) respectively, and/or a light chain with three CDRs comprising the amino acid sequences SGSIASNY (SEQ ID NO:296), EDN (SEQ ID NO:297), and QAWDSNSYV (SEQ ID NO: 2781) respectively;
b) a heavy chain with three CDRs comprising the amino acid sequences GGTFSSYA (SEQ ID NO:164), ISGYNGNT (SEQ ID NO:165), and ARQMKDSGNYWEYYYYGMDV (SEQ ID NO:166) respectively, and/or a light chain with three CDRs comprising the amino acid sequences NIGSES (SEQ ID NO:299), EDR (SEQ ID NO:300), and QVWNPSGSLQYV (SEQ ID NO:301) respectively;
c) a heavy chain with three CDRs comprising the amino acid sequences GYTFTSYG (SEQ ID NO:167), ISTYNGNT (SEQ ID NO:168), and ARDVFGHFDY (SEQ ID NO:169) respectively, and/or a light chain with three CDRs comprising the amino acid sequences SGNIATNY (SEQ ID NO:302), EDN (SEQ ID NO:303), and KSYDDGNHV (SEQ ID NO:304) respectively;
d) a heavy chain with three CDRs comprising the amino acid sequences GFSLTTTGVS (SEQ ID NO:170), IHWDDDK (SEQ ID NO:171), and ASFIMTVYAEYFED (SEQ ID NO: 172) respectively, and/or a light chain with three CDRs comprising the amino acid sequences QSVSSN (SEQ ID NO:305), DVS (SEQ ID NO:306), and QQRGVWPLT (SEQ ID NO:307) respectively;
e) a heavy chain with three CDRs comprising the amino acid sequences GFSLSTSAMC (SEQ ID NO:173), IDWDNDR (SEQ ID NO:174), and AHSPYDSIWGSFRPSVYYFDY (SEQ ID NO:175) respectively, and/or a light chain with three CDRs comprising the amino acid sequences SGSIVSSY (SEQ ID NO:308), EHN (SEQ ID NO:309), and QSYDSQNGV (SEQ ID NO:310) respectively;
f) a heavy chain with three CDRs comprising the amino acid sequences GFTFSDYY (SEQ ID NO:176), ISSSSSDT (SEQ ID NO:177), and AMPTREPAY (SEQ ID NO:178) respectively, and/or a light chain with three CDRs comprising the amino acid sequences SSDLGTYNY (SEQ ID NO:311), DVF (SEQ ID NO:312), and SSYTSSSTYV (SEQ ID NO:313) respectively;
g) a heavy chain with three CDRs comprising the amino acid sequences GFAFSDFP (SEQ ID NO: 179), ISYDGSLK (SEQ ID NO:180), and AREGVSNSRPFDH (SEQ ID NO:181) respectively, and/or a light chain with three CDRs comprising the amino acid sequences SIGTKS (SEQ ID NO: 314), DDS (SEQ ID NO: 2782), and QVWESDDDDLV (SEQ ID NO:316) respectively;
h) a heavy chain with three CDRs comprising the amino acid sequences GFTFSSYA (SEQ ID NO:182), ISSNGGST (SEQ ID NO:183), and TRDLWSGSADSFDI (SEQ ID NO:184) respectively, and/or a light chain with three CDRs comprising the amino acid sequences SLRRYY (SEQ ID NO: 317), GKN (SEQ ID NO:318), and NSRDISDNQWQWI (SEQ ID NO:319) respectively;
i) a heavy chain with three CDRs comprising the amino acid sequences GFPFNAYY (SEQ ID NO:185), INQDGSEK (SEQ ID NO:186), and ARLYWWGMDV (SEQ ID NO:187) respectively, and/or a light chain with three CDRs comprising the amino acid sequences SSDVGGYKY (SEQ ID NO:320), DVN (SEQ ID NO:321), and SSYTGRMNLYV (SEQ ID NO:322) respectively;
j) a heavy chain with three CDRs comprising the amino acid sequences GFTFDDYA (SEQ ID NO:188), IDWNSGVI (SEQ ID NO:189), and AKDAYSYGFLGAFDI (SEQ ID NO:190) respectively, and/or a light chain with three CDRs comprising the amino acid sequences NIRTKG (SEQ ID NO:323), YAS (SEQ ID NO:324), and QVWDSSSDLVV (SEQ ID NO:325) respectively;
k) a heavy chain with three CDRs comprising the amino acid sequences GFTFDDYA (SEQ ID NO:191), ISWNSGSI (SEQ ID NO:192), and ARDWWGSIDH (SEQ ID NO:193) respectively, and/or a light chain with three CDRs comprising the amino acid sequences SSDVGGYDY (SEQ ID NO:326), DVS (SEQ ID NO:327), and SSYTSSSPVV (SEQ ID NO:328) respectively;
l) a heavy chain with three CDRs comprising the amino acid sequences GGSISSSNW (SEQ ID NO:194), IYHSGST (SEQ ID NO:195), and ARRGGTYHRGAFDI (SEQ ID NO:196) respectively, and/or a light chain with three CDRs comprising the amino acid sequences SRDVGSYDL (SEQ ID NO:329), EGS (SEQ ID NO:330), and SSYTSSNSLV (SEQ ID NO:331) respectively;
m) a heavy chain with three CDRs comprising the amino acid sequences GASISNSF (SEQ ID NO:197), TSYSGNS (SEQ ID NO:198), and ARREWIKGHFDY (SEQ ID NO:199) respectively, and/or a light chain with three CDRs comprising the amino acid sequences GGSIASNY (SEQ ID NO:332), EDN (SEQ ID NO:333), and QSYDSSNPVV (SEQ ID NO:334) respectively;
n) a heavy chain with three CDRs comprising the amino acid sequences GGSFTTHS (SEQ ID NO:200), ILPGGAT (SEQ ID NO:201), and ARGPGILSY (SEQ ID NO:202) respectively, and/or a light chain with three CDRs comprising the amino acid sequences SSIGSND (SEQ ID NO:335), SNN (SEQ ID NO:336), and AWDDSLSAVV (SEQ ID NO:337) respectively;
o) a heavy chain with three CDRs comprising the amino acid sequences GGSFRTHS (SEQ ID NO:203), IHHSGAT (SEQ ID NO:204), and ARGPGILSY (SEQ ID NO:205) respectively, and/or a light chain with three CDRs comprising the amino acid sequences SSNIGSNT (SEQ ID NO:338), INN (SEQ ID NO:339), and AEWYDSLNVHYV (SEQ ID NO:340) respectively;
p) a heavy chain with three CDRs comprising the amino acid sequences GGSIRTHS (SEQ ID NO:206), IHHSGAT (SEQ ID NO:207), and ARGPGILSY (SEQ ID NO:208) respectively, and/or a light chain with three CDRs comprising the amino acid sequences SSNIGSNT (SEQ ID NO:341), INN (SEQ ID NO:342), and AECYDSLNDHYV (SEQ ID NO:343) respectively;
q) a heavy chain with three CDRs comprising the amino acid sequences GGSIRTHS (SEQ ID NO:209), IHHSGAT (SEQ ID NO:210), and GRGPGILSY (SEQ ID NO:211) respectively, and/or a light chain with three CDRs comprising the amino acid sequences SSNIGSNT (SEQ ID NO:344), SNN (SEQ ID NO:345), and AAWDDSLNVHYV (SEQ ID NO:346) respectively;
r) a heavy chain with three CDRs comprising the amino acid sequences GYSFTSYW (SEQ ID NO:212), IYPGDSDT (SEQ ID NO:213), and ARQGDGGGYDY (SEQ ID NO:214) respectively, and/or a light chain with three CDRs comprising the amino acid sequences SSNIGSNP (SEQ ID NO:347), NNN (SEQ ID NO:348), and AAWDDSLNGL (SEQ ID NO:349) respectively;
s) a heavy chain with three CDRs comprising the amino acid sequences RYSFSNYW (SEQ ID NO:215), IYPYDSDT (SEQ ID NO:216), and ARQGSSQSFDI (SEQ ID NO:217) respectively, and/or a light chain with three CDRs comprising the amino acid sequences SLRSYY (SEQ ID NO:350), QDS (SEQ ID NO: 2783), and QAWDSNSYV (SEQ ID NO: 2781) respectively;
t) a heavy chain with three CDRs comprising the amino acid sequences GYSFTSYW (SEQ ID NO:218), IYPGDSDT (SEQ ID NO:219), and ARRRGSAAAFDT (SEQ ID NO:220) respectively, and/or a light chain with three CDRs comprising the amino acid sequences SSNIGSNP (SEQ ID NO:353), DNN (SEQ ID NO:354), and EAWDDSLSGPV (SEQ ID NO:355) respectively;
u) a heavy chain with three CDRs comprising the amino acid sequences GYSFTSYW (SEQ ID NO:221), IYPGDSDT (SEQ ID NO:222), and ARTTYSYGSFDY (SEQ ID NO:223) respectively, and/or a light chain with three CDRs comprising the amino acid sequences SSNIGGNS (SEQ ID NO:356), RNN (SEQ ID NO:357), and AAWDDSLNGWV (SEQ ID NO:358) respectively; or
v) a heavy chain with three CDRs comprising the amino acid sequences GDSVTSNSAA (SEQ ID NO:224), TYYSSKWYN (SEQ ID NO:225), and ARGWLRLSFDP (SEQ ID NO:226) respectively, and/or a light chain with three CDRs comprising the amino acid sequences SGSIASNY (SEQ ID NO:359), EDN (SEQ ID NO:360), and QSYDPNNHGVV (SEQ ID NO:361) respectively.

In other embodiments, the scFv antibody comprises: a) a heavy chain with three CDRs comprising the amino acid sequences GFSLSTSGVG (SEQ ID NO:754), IYWDDDK (SEQ ID NO:755), and ARISGSGYFYPFDI (SEQ ID NO:756) respectively, and/or a light chain with three CDRs comprising the amino acid sequences SGSIASNY (SEQ ID NO:802), EDN (SEQ ID NO:803), and QSYDSSNLWV (SEQ ID NO:804) respectively; b) a heavy chain with three CDRs comprising the amino acid sequences GDSVSSNSAA (SEQ ID NO:757), TYYRSRWYN (SEQ ID NO:758), and AREIRGFDY (SEQ ID NO:759) respectively, and/or a light chain with three CDRs comprising the amino acid sequences SSDVGAYNF (SEQ ID NO:805), DFN (SEQ ID NO:806), and SSYAGSNNFDVV (SEQ ID NO:807) respectively; c) a heavy chain with three CDRs comprising the amino acid sequences GFTFGDYA (SEQ ID NO:760), IRSKAYGGTT (SEQ ID NO:761), and TTADDDMDV (SEQ ID NO:762) respectively, and/or a light chain with three CDRs comprising the amino acid sequences SGTIASNY (SEQ ID NO:808), EDN (SEQ ID NO:809), and QSYDTSNHYV (SEQ ID NO:810) respectively; d) a heavy chain with three CDRs comprising the amino acid sequences GFTFSNYG (SEQ ID NO:763), IWERGSKK (SEQ ID NO:764), and AREGISMTGAEYFQH (SEQ ID NO:765) respectively, and/or a light chain with three CDRs comprising the amino acid sequences SSNIGAGYD (SEQ ID NO:811), GTN (SEQ ID NO:812), and QSYDNSLTDPYV (SEQ ID NO:813) respectively; e) a heavy chain with three CDRs comprising the amino acid sequences GFTFDDYA (SEQ ID NO:766), IDWNSGVI (SEQ ID NO:767), and AKDIGPGGSGSYYAFDI (SEQ ID NO:768) respectively, and/or a light chain with three CDRs comprising the amino acid sequences SSDVGGSKY (SEQ ID NO:814), DVT (SEQ ID NO:815), and AAWDDSLNGVV (SEQ ID NO:816) respectively; f) a heavy chain with three CDRs comprising the amino acid sequences GFSFSRYG (SEQ ID NO:769), IRHDGSKK (SEQ ID NO:770), and AKDGRLEAALDD (SEQ ID NO:771) respectively, and/or a light chain with three CDRs comprising the amino acid sequences SGSIANNF (SEQ ID NO:817), EDN (SEQ ID NO:818), and QSYDSSNLV (SEQ ID NO:819) respectively; g) a heavy chain with three CDRs comprising the amino acid sequences GYSFTSYW (SEQ ID NO:772), IYPGDSDT (SEQ ID NO:773), and ARRGDLDAFDI (SEQ ID NO:774) respectively, and/or a light chain with three CDRs comprising the amino acid sequences SANIGSNA (SEQ ID NO:820), GNT (SEQ ID NO:821), and AAWDDSLNGYV (SEQ ID NO:822) respectively; h) a heavy chain with three CDRs comprising the amino acid sequences GYRLSDYY (SEQ ID NO:775), IKQDGSEK (SEQ ID NO:776), and ARVRGWSRGYFDY (SEQ ID NO:777) respectively, and/or a light chain with three CDRs comprising the amino acid sequences SGSIASNY (SEQ ID NO:823), EDN (SEQ ID NO:824), and QSYDSSNHWV (SEQ ID NO:825) respectively; i) a heavy chain with three CDRs comprising the amino acid sequences GFTFDDYA (SEQ ID NO:778), ISWNSGSI (SEQ ID NO:779), and ARDWWGSIDH (SEQ ID NO:780) respectively, and/or a light chain with three CDRs comprising the amino acid sequences SSDVGGYDY (SEQ ID NO:826), DVS (SEQ ID NO:827), and SSYTSSSPVV (SEQ ID NO:828) respectively; j) a heavy chain with three CDRs comprising the amino acid sequences GFTFSHYD (SEQ ID NO:781), IGYDGTNL (SEQ ID NO:782), and ARAANYYDSSGYGRADAF (SEQ ID NO:2784) respectively, and/or a light chain with three CDRs comprising the amino acid sequences TGSIAGNY (SEQ ID NO:829), DDN (SEQ ID NO:830), and QSYDSGNRGV (SEQ ID NO:831) respectively; k) a heavy chain with three CDRs comprising the amino acid sequences GGTFSTYG (SEQ ID NO:784), IIPSLGIP (SEQ ID NO:785), and ARENIDLATNDF (SEQ ID NO:786) respectively, and/or a light chain with three CDRs comprising the amino acid sequences SRDIGAYGY (SEQ ID NO:832), EVR (SEQ ID NO:833), and SSYTSSSTLDVV (SEQ ID NO:834) respectively; 1) a heavy chain with three CDRs comprising the amino acid sequences GGTFSSSG (SEQ ID NO:787), IIPMLGTP (SEQ ID NO:788), and ARDGGNYDY (SEQ ID NO:789) respectively, and/or a light chain with three CDRs comprising the amino acid sequences SSNIGRNA (SEQ ID NO:835), SNN (SEQ ID NO:836), and SAWDTSLSTWV (SEQ ID NO:837) respectively; m) a heavy chain with three CDRs comprising the amino acid sequences GFTFSSYW (SEQ ID NO:790), IKQDGSEK (SEQ ID NO:791), and ARGFYYYGAFDI (SEQ ID NO:792) respectively, and/or a light chain with three CDRs comprising the amino acid sequences SGSIASNY (SEQ ID NO:838), EDN (SEQ ID NO:839), and QSYDSSNHWV (SEQ ID NO:840) respectively; n) a heavy chain with three CDRs comprising the amino acid sequences GFTFDDYA (SEQ ID NO:793), IDWNSGVI (SEQ ID NO:794), and AKDAYSYGFLGAFDI (SEQ ID NO:795) respectively, and/or a light chain with three CDRs comprising the amino acid sequences NIRTKG (SEQ ID NO:841), YAS (SEQ ID NO:842), and QVWDSSSDLVV (SEQ ID NO:843) respectively; o) a heavy chain with three CDRs comprising the amino acid sequences GYSFTGSH (SEQ ID NO:796), INPDSGVI (SEQ ID NO:797), and ARDKAIGYVWALDY (SEQ ID NO:798) respectively, and/or a light chain with three CDRs comprising the amino acid sequences SSDVGTYNR (SEQ ID NO:844), EVS (SEQ ID NO:845), and SSYTRTFTYV (SEQ ID NO:846) respectively; or p) a heavy chain with three CDRs comprising the amino acid sequences GASISNSF (SEQ ID NO:799), TSYSGNS (SEQ ID NO:800), and ARREWIKGHFDY (SEQ ID NO:801) respectively, and/or a light chain with three CDRs comprising the amino acid sequences GGSIASNY (SEQ ID NO:847), EDN (SEQ ID NO:848), and QSYDSSNPVV (SEQ ID NO:849) respectively. In embodiments, the scFv antibody comprises a heavy chain with three CDRs comprising the amino acid sequences GFSLTTSGVS (SEQ ID NO:983), IHWDDDK (SEQ ID NO:984), and ASFIMTVYAEYFED (SEQ ID NO:985) respectively, and/or a light chain with three CDRs comprising the amino acid sequences QSVSSN (SEQ ID NO:986), DVS (SEQ ID NO:987), and QQRGAWPLT (SEQ ID NO:988) respectively

In other embodiments, the scFv antibody comprises:
a. a V_{H} amino acid sequence having SEQ ID NO: 1, and a V_{L} amino acid sequence having SEQ ID NO: 2;
b. a V_{H} amino acid sequence having SEQ ID NO: 3, and a V_{L} amino acid sequence having SEQ ID NO: 4;
c. a V_{H} amino acid sequence having SEQ ID NO: 5, and a V_{L} amino acid sequence having SEQ ID NO: 6;
d. a V_{H} amino acid sequence having SEQ ID NO: 7, and a V_{L} amino acid sequence having SEQ ID NO: 8;
e. a V_{H} amino acid sequence having SEQ ID NO: 9, and a V_{L} amino acid sequence having SEQ ID NO: 10;
f. a V_{H} amino acid sequence having SEQ ID NO: 11, and a V_{L} amino acid sequence having SEQ ID NO: 12;
g. a V_{H} amino acid sequence having SEQ ID NO: 13, and a V_{L} amino acid sequence having SEQ ID NO: 14;
h. a V_{H} amino acid sequence having SEQ ID NO: 19, and a V_{L} amino acid sequence having SEQ ID NO: 20;
i. a V_{H} amino acid sequence having SEQ ID NO: 21, and a V_{L} amino acid sequence having SEQ ID NO: 22;
j. a V_{H} amino acid sequence having SEQ ID NO: 23, and a V_{L} amino acid sequence having SEQ ID NO: 24;
k. a V_{H} amino acid sequence having SEQ ID NO: 25, and a V_{L} amino acid sequence having SEQ ID NO: 26;
l. a V_{H} amino acid sequence having SEQ ID NO: 29, and a V_{L} amino acid sequence having SEQ ID NO: 30;
m. a V_{H} amino acid sequence having SEQ ID NO: 31, and a V_{L} amino acid sequence having SEQ ID NO: 32;
n. a V_{H} amino acid sequence having SEQ ID NO: 33, and a V_{L} amino acid sequence having SEQ ID NO: 34;
o. a V_{H} amino acid sequence having SEQ ID NO: 35, and a V_{L} amino acid sequence having SEQ ID NO: 36;
p. a V_{H} amino acid sequence having SEQ ID NO: 37, and a V_{L} amino acid sequence having SEQ ID NO: 38;
q. a V_{H} amino acid sequence having SEQ ID NO: 39, and a V_{L} amino acid sequence having SEQ ID NO: 40;
r. a V_{H} amino acid sequence having SEQ ID NO: 41, and a V_{L} amino acid sequence having SEQ ID NO: 42;
s. a V_{H} amino acid sequence having SEQ ID NO: 43, and a V_{L} amino acid sequence having SEQ ID NO: 44; or
t. a V_{H} amino acid sequence having SEQ ID NO: 47, and a V_{L} amino acid sequence having SEQ ID NO: 48.

In some embodiments, the scFv antibody comprises: (a) a V_{H} amino acid sequence having SEQ ID NO: 15, and a V_{L} amino acid sequence having SEQ ID NO: 16; (b) a V_{H} amino acid sequence having SEQ ID NO: 17, and a V_{L} amino acid sequence having SEQ ID NO: 18; or (c) a V_{H} amino acid sequence having SEQ ID NO: 27, and a V_{L} amino acid sequence having SEQ ID NO: 28. In other embodiments, the scFv antibody comprises:
a. a V_{H} amino acid sequence having SEQ ID NO: 49, and a V_{L} amino acid sequence having SEQ ID NO: 50;
b. a V_{H} amino acid sequence having SEQ ID NO: 51, and a V_{L} amino acid sequence having SEQ ID NO: 52;
c. a V_{H} amino acid sequence having SEQ ID NO: 53, and a V_{L} amino acid sequence having SEQ ID NO: 54;
d. a V_{H} amino acid sequence having SEQ ID NO: 55, and a V_{L} amino acid sequence having SEQ ID NO: 56;
e. a V_{H} amino acid sequence having SEQ ID NO: 57, and a V_{L} amino acid sequence having SEQ ID NO: 58;
f. a V_{H} amino acid sequence having SEQ ID NO: 59, and a V_{L} amino acid sequence having SEQ ID NO: 60;
g. a V_{H} amino acid sequence having SEQ ID NO: 61, and a V_{L} amino acid sequence having SEQ ID NO: 62;
h. a V_{H} amino acid sequence having SEQ ID NO: 63, and a V_{L} amino acid sequence having SEQ ID NO: 64;
i. a V_{H} amino acid sequence having SEQ ID NO: 65, and a V_{L} amino acid sequence having SEQ ID NO: 66;
j. a V_{H} amino acid sequence having SEQ ID NO: 67, and a V_{L} amino acid sequence having SEQ ID NO: 68;
k. a V_{H} amino acid sequence having SEQ ID NO: 69, and a V_{L} amino acid sequence having SEQ ID NO: 70;
l. a V_{H} amino acid sequence having SEQ ID NO: 71, and a V_{L} amino acid sequence having SEQ ID NO: 72;
m. a V_{H} amino acid sequence having SEQ ID NO: 73, and a V_{L} amino acid sequence having SEQ ID NO: 74;
n. a V_{H} amino acid sequence having SEQ ID NO: 75, and a V_{L} amino acid sequence having SEQ ID NO: 76;
o. a V_{H} amino acid sequence having SEQ ID NO: 77, and a V_{L} amino acid sequence having SEQ ID NO: 78;
p. a V_{H} amino acid sequence having SEQ ID NO: 79, and a V_{L} amino acid sequence having SEQ ID NO: 80;
q. a V_{H} amino acid sequence having SEQ ID NO: 81, and a V_{L} amino acid sequence having SEQ ID NO: 82;
r. a V_{H} amino acid sequence having SEQ ID NO: 83, and a V_{L} amino acid sequence having SEQ ID NO: 84;
s. a V_{H} amino acid sequence having SEQ ID NO: 85, and a V_{L} amino acid sequence having SEQ ID NO: 86;
t. a V_{H} amino acid sequence having SEQ ID NO: 87, and a V_{L} amino acid sequence having SEQ ID NO: 88;
u. a V_{H} amino acid sequence having SEQ ID NO: 89, and a V_{L} amino acid sequence having SEQ ID NO: 90; or
v. a V_{H} amino acid sequence having SEQ ID NO: 91, and a V_{L} amino acid sequence having SEQ ID NO: 92.

In other embodiments, the scFv antibody comprises: a) a VH amino acid sequence having SEQ ID NO: 722, and a VL amino acid sequence having SEQ ID NO: 723; b) a VH amino acid sequence having SEQ ID NO: 724, and a VL amino acid sequence having SEQ ID NO: 725; c) a VH amino acid sequence having SEQ ID NO: 726, and a VL amino acid sequence having SEQ ID NO: 727; d) a VH amino acid sequence having SEQ ID NO: 728, and a VL amino acid sequence having SEQ ID NO: 729; e) a VH amino acid sequence having SEQ ID NO: 730, and a VL amino acid sequence having SEQ ID NO: 731; f) a VH amino acid sequence having SEQ ID NO: 732, and a VL amino acid sequence having SEQ ID NO: 733; g) a VH amino acid sequence having SEQ ID NO: 734, and a VL amino acid sequence having SEQ ID NO: 735; h) a VH amino acid sequence having SEQ ID NO: 736, and a VL amino acid sequence having SEQ ID NO: 737; i) a VH amino acid sequence having SEQ ID NO: 738, and a VL amino acid sequence having SEQ ID NO: 739; j) a VH amino acid sequence having SEQ ID NO: 740, and a VL amino acid sequence having SEQ ID NO: 741; k) a VH amino acid sequence having SEQ ID NO: 742, and a VL amino acid sequence having SEQ ID NO: 743; 1) a VH amino acid sequence having SEQ ID NO: 744, and a VL amino acid sequence having SEQ ID NO: 745; m) a VH amino acid sequence having SEQ ID NO: 746, and a VL amino acid sequence having SEQ ID NO: 747; n) a VH amino acid sequence having SEQ ID NO: 748, and a VL amino acid sequence having SEQ ID NO: 749; o) a VH amino acid sequence having SEQ ID NO: 750, and a VL amino acid sequence having SEQ ID NO: 751; or p) a VH amino acid sequence having SEQ ID NO: 752, and a VL amino acid sequence having SEQ ID NO: 753. In embodiments, the scFv antibody comprises a VH amino acid sequence having SEQ ID NO: 981, and a VL amino acid sequence having SEQ ID NO: 982.

An aspect of the invention is directed to methods of preventing a disease or disorder caused by a Severe Acute Respiratory Syndrome coronavirus (SARS-CoV2). In some embodiments, the method comprises administering to a subject at risk of suffering from the disease or disorder, a therapeutically effective amount of the monoclonal antibody described herein or the scFv antibody described herein. In some embodiments, the method further comprises administering an anti-viral drug, a viral entry inhibitor, a viral attachment inhibitor, or a combination thereof. In some embodiments, the method comprises administering two or more antibodies specific to SARS-CoV2. In some embodiments, the antibody is administered prior to or after exposure to SARS-CoV2. In other embodiments, the antibody is administered at a dose sufficient to neutralize the SARS-CoV2.

An aspect of the invention is directed to methods of delaying the onset of one or more symptoms of a SARS-CoV2 infection. In some embodiments, the method comprises administering to a subject at risk of suffering from the disease or disorder, a therapeutically effective amount of the monoclonal antibody described herein or the scFv antibody described herein. In some embodiments, the method further comprises administering an anti-viral drug, a viral entry inhibitor, a viral attachment inhibitor, or a combination thereof. In some embodiments, the method comprises administering two or more antibodies specific to SARS-CoV2. In some embodiments, the antibody is administered prior to or after exposure to SARS-CoV2. In other embodiments, the antibody is administered at a dose sufficient to neutralize the SARS-CoV2.

An aspect of the invention is directed to compositions comprising the monoclonal antibody described herein or the scFv antibody described herein, and a carrier.

An aspect of the invention is directed to methods of detecting the presence of SARS-CoV2 in a sample. In some embodiments, the method comprising: (a) contacting the sample with the monoclonal antibody described herein or the scFv antibody described herein; and detecting the presence or absence of an antibody-antigen complex, thereby detecting the presence of SARS-CoV2 in a sample. In some embodiments, the detecting occurs in vivo. In other embodiments, the sample is obtained from blood, hair, cheek scraping, saliva, biopsy, or semen.

Unless otherwise defined, all technical and scientific terms used herein can have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and are not intended to be limiting.

Other features and advantages of the invention will be apparent from and are encompassed by the following detailed description and claims.

### BRIEF DESCRIPTION OF THE DRAWINGS

FIG. 1A-FIG. 1P shows the amino acid sequences of the heavy chain and light chain regions of the antibodies directed to SARS-COV-2. Figure discloses SEQ ID NOS 1564-1931, respectively in order of columns (CDR1, CDR2, CDR3, full-length sequence).

FIG. 2 shows a panning strategy (soluble protein).

FIG. 3 shows screening results.

FIG. 4 shows purified phage binding curves (RBD-Fc). The curves are made by coating plates with 1 μg/ml of either SARS-COV-2 RBD-Fc or IL2-Fc (negative control) or blocking buffer only. Phage binding was detected by anti-M13-HRP; Negative curves plotted with the positive samples are against blocking buffer; Negative curves on the slides after are against IL2-Fc (1 μg/ml).

FIG. 5 shows EC50 values for purified phage against RBD-Fc. Red names had ambiguous curve fitting. Consult graphs for data reliability.

FIG. 6 shows Fc coat negative binding curves.

FIG. 7 shows purified phage binding against S1 protein. Negatives are also graphed.

FIG. 8 shows SARS-RBD-Fc ACE2 binding curve. These curves are made by coating plates with 1 μg/ml of either SARS-COV-2 RBD-Fc or IL2-Fc (negative control). Phage binding was detected by anti-M13-HRP; negative curves are plotted for each phage on the same graph.

FIG. 9 shows anti-RBD competition with ACE2. The red box on plate 1 shows exemplary clones of interest. These clones appear to demonstrate at least a partial ability to block RBD-ACE2 binding.

FIG. 10 shows a detailed look at the 7 anti-RBD clones that shows differential ELISA signal in blocking experiment. In this experiment, if the red bar is below that of the purple bar, it indicates that there is competition of the phage with ACE2.

FIG. 11 shows a RBD phage competition curve.

FIG. 12 shows the amino acid sequences of the heavy chain and light chain regions of the antibodies directed to SARS-COV-2. The asterisks are amber/stop codons. In the TG1 bacterial cells, they are mutated such that the TAG stop codon is read as a Q (glutamine). When the IMGT numbering is used to break the DNA sequence down into FW/CDRs, the system does not recognize an amber suppressor so a stop codon is assumed, but in the phage the codon is read as a Q. The sequences are later re-cloned such that the TAG is changed to the codons for Q. The periods are also from the IMGT system. In the numbering system they use, each FW/CDR needs to have a certain number of residues, so the periods are just used as gaps to make the beginning and the end of the segments fit their numbering scheme. Figure discloses SEQ ID NOS 1564-1931, respectively in order of columns (CDR1, CDR2, CDR3, full-length sequence).

FIG. 13 is a table of KD measurements. KD values were measured on Octet with SA sensors. All abs are scFv-Fc format except for CR3022. Sensors were coated with 2.5 ug/ml Biotinylated SARS-CoV-2 S1 protein (ACRO, S1N-C82E8). Abs were run at 3 concentrations, 25-12.5-6.25 nM and the kinetic parameters were calculated by linking the three curves. Control sensor was coated with biotinylated PD1 and 25 nM antibody was allowed to bind.

FIG. 14 shows graphs of kinetic measurements.

FIG. 15 shows the nucleic acid sequences of the heavy chain and light chain regions of antibodies directed to SARS-COV-2. Figure discloses SEQ ID NOS 1932-2291, respectively in order of columns (CDR1, CDR2, CDR3, full-length sequence).

FIG. 16 shows a phylogenetic tree of the coronavirus family and schematics of the viruses taken from Li, F. (2016). Structure, Function, and Evolution of Coronavirus Spike Proteins. Annu Rev Virol. http://doi.org/doi:10.1146/annurev-virology-110615-042301. The figure is an introduction to coronaviruses and their spike proteins. (a) Classification of coronaviruses. Representative coronaviruses in each genus are human coronavirus NL63 (HCoV-NL63), porcine transmissible gastroenteritis coronavirus (TGEV), porcine epidemic diarrhea coronavirus (PEDV), and porcine respiratory coronavirus (PRCV) in the genus Alphacoronavirus; severe acute respiratory syndrome coronavirus (SARS-CoV), Middle East respiratory syndrome coronavirus (MERS-CoV), bat coronavirus HKU4, mouse hepatitis coronavirus (MHV), bovine coronavirus (BCoV), and human coronavirus OC43 in the genus Betacoronavirus; avian infectious bronchitis coronavirus (IBV) in the genus Gammacoronavirus; and porcine deltacoronavirus (PdCV) in the genus Deltacoronavirus. (b) Schematic of the overall structure of prefusion coronavirus spikes. Shown are the receptor-binding subunit S1, the membrane-fusion subunit S2, the transmembrane anchor (TM), the intracellular tail (IC), and the viral envelope. (c) Schematic of the domain structure of coronavirus spikes, including the S1 N-terminal domain (S1-NTD), the S1 C-terminal domain (S1-CTD), the fusion peptide (FP), and heptad repeat regions N and C (HR-N and HR-C). Scissors indicate two proteolysis sites in coronavirus spikes. (d) Summary of the structures and functions of coronavirus spikes. Host receptors recognized by either of the S1 domains are angiotensin-converting enzyme 2 (ACE2), aminopeptidase N (APN), dipeptidyl peptidase 4 (DPP4), carcinoembryonic antigen-related cell adhesion molecule 1 (CEACAMI), and sugar. The available crystal structures of S1 domains and S2 HRs are shown. Their PDB IDs are 3KBH for HCoV-NL63 S1-CTD; 4F5C for PRCV S1-CTD; 2AJF for SARS-CoV S1-CTD; 4KR0 for MERS-CoV S1-CTD; 3R4D for MHIV S1-NTD; 4H14 for BCoV S1-NTD; 2IEQ for HCoV-NL63 HRs; 1WYY for SARS-CoV HRs; 4NJL for MERS-CoV HRs; and 1WDF for MHV HRs.

FIG. 17 is a schematic of the coronavirus structure adapted from Li, F. (2016). Structure, Function, and Evolution of Coronavirus Spike Proteins. Annu Rev Virol. http://doi.org/doi:10.1146/annurev-virology-110615-042301.

FIG. 18 is a schematic of the structure of the 2019-nCoV S in the prefusion conformation adapted from Daniel Wrapp et al. Science 2020; 367:1260-1263. (A) Schematic of 2019-nCoV S primary structure colored by domain. Domains that were excluded from the ectodomain expression construct or could not be visualized in the final map are colored white. SS, signal sequence; S2′, S2′ protease cleavage site; FP, fusion peptide; HR1, heptad repeat 1; CH, central helix; CD, connector domain; HR2, heptad repeat 2; TM, transmembrane domain; CT, cytoplasmic tail. Arrows denote protease cleavage sites. (B) Side and top views of the prefusion structure of the 2019-nCoV S protein with a single RBD in the up conformation. The two RBD down protomers are shown as cryo-EM density in either white or gray and the RBD up protomer is shown in ribbons colored corresponding to the schematic in (A).

FIG. 19 is a schematic of ribbon diagrams showing the structural comparison between 2019-nCoV S and SARS-CoV Sadapted from Daniel Wrapp et al. Science 2020; 367:1260-1263. (A) Single protomer of 2019-nCoV S with the RBD in the down conformation (left) is shown in ribbons colored according to FIG. 1 of Wrapp et al. Science 2020; 367:1260-1263. A protomer of 2019-nCoV S in the RBD up conformation is shown (center) next to a protomer of SARS-CoV S in the RBD up conformation (right), displayed as ribbons and colored white (PDB ID: 6CRZ). (B) RBDs of 2019-nCoV and SARS-CoV aligned based on the position of the adjacent NTD from the neighboring protomer. The 2019-nCoV RBD is colored green and the SARS-CoV RBD is colored white. The 2019-nCoV NTD is colored blue. (C) Structural domains from 2019-nCoV S have been aligned to their counterparts from SARS-CoV S as follows: NTD (top left), RBD (top right), SD1 and SD2 (bottom left), and S2 (bottom right).

FIG. 20 is a graph showing 2019-nCoV S binds human ACE2 with high affinity adapted from Daniel Wrapp et al. Science 2020; 367:1260-1263. Surface plasmon resonance sensorgram shows the binding kinetics for human ACE2 and immobilized 2019-nCoV S. Data are shown as black lines, and the best fit of the data to a 1:1 binding model is shown in red.

FIG. 21 shows the antigenicity of the 2019-nCoV RBD adapted from Daniel Wrapp et al. Science 2020; 367:1260-1263. (A) SARS-CoV RBD shown as a white molecular surface (PDB ID: 2AJF), with residues that vary in the 2019-nCoV RBD colored red. The ACE2-binding site is outlined with a black dashed line. (B) Biolayer interferometry sensorgram showing binding to ACE2 by the 2019-nCoV RBD-SD1. Binding data are shown as a black line, and the best fit of the data to a 1:1 binding model is shown in red. (C) Biolayer interferometry to measure cross-reactivity of the SARS-CoV RBD-directed antibodies S230, m396, and 80R. Sensor tips with immobilized antibodies were dipped into wells containing 2019-nCoV RBD-SD1, and the resulting data are shown as a black line.

FIG. 22 shows tables of the panning process conducted to identify the SARS-CoV2 antibodies described herein.

FIG. 23 shows a table of the screening process conducted to identify the SARS-CoV2 antibodies described herein. SARS2 was screened via ELISA, ACE2sol was screened against 293T-ACE2 stably transfected cells.

FIG. 24 is a binding curve showing SARS-RBD-Fc binding to ACE2.

FIG. 25 outlines the Panning plan.

FIG. 26 is a graph showing virus infection. GD03 SARS and SARS2 pseudovirus was generated by transfecting LentiX-293T cells. ACE2+ target cells were incubated with varying dilutions of the pseudovirus supernatant for 48 hours before cell lysis and luciferase detection. The SARS2 pseudovirus displays decreased infection compared to the GD03 SARS strain which could be explained by either low production titers or decreased viral entry into the target cells. However, the values for SARS2 are above baseline and can be used for introductory pseudovirus neutralization assays.

FIG. 27 shows optimization of the pseudovirus.

FIG. 28 is a graph showing SARS/SARS-CoV2 pseudovirus infection of 293T cells transduced with ACE2. Two SARS-CoV2 spike pseudovirus constructs were used, WT spike and one with the end of the intraviron domain replaced with a gp41 tail. Two preps of pseudovirus were also used, one made in 150 mm plates with 3 day transfection, the second done in 100 mm plates with 2 day incubation (cells were all floating after 2 days). Transfected with Lipofectamine 3000. 10,000 transduced 293T-ACE2 cells were cultured O/N. The next day pseudovirus supernatant was added to the sample in serial 2×dilutions, starting with straight supernatant in the top well. Plates were incubated for 48 hours before the supernatant was removed and cells were lysed with Promega passive lysis buffer. After equilibration period, Promega BioGlow luciferase reagent was added and the plate was read. Interval time was 0.5 sec, and the gain adjustment was at 40%

FIG. 29 shows a table of the germline assignments for the first set of SARS-CoV2 antibodies identified.

FIG. 30 shows sequence alignments of SARS-CoV2 antibodies identified. Figure discloses SEQ ID NOS 2292-2311, respectively in order of appearance.

FIG. 31 shows a table of the binding affinities for the first set of SARS-CoV2 antibodies identified. KD values measured on Octet with SA (streptavidin) sensors. All abs are scFv-Fc format except for CR3022. Sensors were coated with 2.5 ug/ml Biotinylated SARS-CoV-2 S1 protein (ACRO, S1N-C82E8). Abs were run at 3 concentrations, 25-12.5-6.25 nM and the kinetic parameters were calculated by linking the three curves. Control sensor was coated with biotinylated PD1 and 25 nM antibody was allowed to bind

FIG. 32 shows binding sensorgrams of the first set of SARS-CoV2 antibodies identified.

FIG. 33 outlines the competition assay protocol used for the first set of SARS-CoV2 antibodies identified.

FIG. 34 shows a graph of a saturation test. SA sensor loaded with S1-biotin (2.5 ug/ml, ACRO). Sensors were then dipped into wells containing a 250 nM ab solution and allowed to bind for 10 minutes. Following a short baseline in PBST, sensors were returned to the ab well to see if there was further binding. As demonstrated here, return to the ab well does not lead to additional binding, indicating that the antibodies are saturating the receptors at 250 nM.

FIG. 35 shows sensorgrams of the first set of SARS-CoV2 antibodies identified. Only the baseline followed by 2nd antibody step is shown here. Each sensor is saturated with an antibody (sensor key below) and after a short baseline is added to wells containing the 2nd competing antibody. The antibody listed on each graph is the competing antibody. The light green lines are sensors loaded with S1, but no 1st antibody (shows maximal binding). Each set also has a “self” competition control, i.e. in Ab 7, the pink line is the competition of a sensor saturated with 250 nM Ab 7, followed by competition in a well with 125 nM Ab 7. Based on these results, Ab 7 and 12 would fall into one bin and Ab2-2, 2-7, 2-10 would fall into the epitope recognized by CR3022.

FIG. 36 shows a table of the competition matrix. In the matrix, the vertical names are the 1st antibody, while the horizontal names are the 2nd/competing antibody. Boxes highlighted in red are considered blocking.

FIG. 37 shows a graph of ACE2 competition. Competition was conducted with ACE2, however protein quantity was limited and not a high enough concentration was used (only used ˜85 nM). No antibody control shows maximal ACE2 binding to S1 loaded sensors. The red line below that is CR3022, which is not reported to block ACE2 binding. All of the antibodies are below the CR3022 line with Ab 12, Ab 2-7, and Ab 2-10 being particularly flat.

FIG. 38 is a table of germline assignments for additional SARS-CoV2 antibodies identified.

FIG. 39 shows sequence alignments of additional SARS-CoV2 antibodies identified. Figure discloses SEQ ID NOS 2312-2331, respectively in order of appearance.

FIG. 40 is a table of the germline references for the additional SARS-CoV2 antibodies identified.

FIG. 41 is a table of the kinetics determined for the additional SARS-CoV2 antibodies identified. A couple of the antibodies bind RBD but not S1. Without wishing to be bound by theory, the differences in binding can be between the ACRO protein and Sino protein. All panning was done with Sino based proteins (e.g. Ab 15 and Ab 25). RBD is also smaller than S1, so loading more molecules when doing RBD loading can lead to increased signal.

FIG. 42 is a table of a competition matrix. These studies were conducted in two separate assays, SARS-CoV2 Abs 13 thru 20 were run together and SARS-CoV2 Abs 21 thru 28 were another group. Ab 2-2 was used as a surrogate for CR3022 in both assays. The 1st ab was used at 250 nM (vertical axis) and the 2nd ab was used at 125 nM (horizontal axis). Green shaded boxes are non-competing pairs and red shaded boxes are competing pairs. Shading was done manually since our antibodies have a range of binding characteristics and maximum, unblocked binding could have been below the threshold.

FIG. 43 shows a schematic of an epitope binning matrix for SARS-CoV2 antibodies.

FIG. 44 outlines the master competition with all groups for SARS-CoV2 antibodies.

FIG. 45 shows a schematic showing a table for the master binning for SARS-CoV2 antibodies 1 thru 28. Using data from the previous competition assays, a final competition assay with the 8 antibodies thought to be in separate bins was performed. Green shaded boxes are non-competing pairs, red shaded boxes are competing pairs, and the lighter green are debatable. Shading was done manually since the antibodies have a range of binding characteristics and maximum, unblocked binding could have been below the threshold. The master bins clarified that Ab 28 and Ab 2-10 are both competing for the same epitope. Other antibodies showed interesting binning characteristics. For example, Ab 12 blocks Ab 14 and Ab 19, but Ab 14 does not block Ab 19 in either direction. A more detailed epitope mapping with finer resolution will be conducted to parse out these differences.

FIG. 46 is a graph showing SARS-CoV2 pseudovirus neutralization by anti-SARS-CoV2 scFv-Fcs. 293T-ACE2 cells were used as targets for SARS-CoV-2 pseudovirus. For neutralization, scFv-Fc was mixed with pseudovirus and incubated at RT for 1 hour. scFv-Fcs were used at about 25 nM* and pseudovirus was diluted 2×. After mixing pseudovirus/ab with the cells, the plates were incubated at 37° C. for 48 hours. Cells were then lysed with Promega passive lysis buffer and Promega bioglo luciferase was added.

FIG. 47 shows antibody nucleotide sequences for SARS-CoV-2 antibodies. Figure discloses SEQ ID NOS 2332-2555, respectively in order of columns (CDR1, CDR2, CDR3, full-length sequence).

FIG. 48 shows antibody amino acid sequences for SARS-CoV-2 antibodies. Figure discloses SEQ ID NOS 2556-2779, respectively in order of columns (CDR1, CDR2, CDR3, full-length sequence).

FIG. 49 shows a schematic of a human antibody discovery through pathogenic CoV Outbreaks of SARS, MERS and SARS2.

FIG. 50 shows a schematic of the size and genetic complexity of the Mehta I & II Human scFv-Phage Display Libraries.

FIG. 51 shows ribbon diagrams for Structural Basis of Neutralization and In Vivo Protection by 80R Antibody.

FIG. 52 shows Mutant MERS-CoVs were assigned to three epitope groups. Four escape mutants were chosen for cross neutralization assay.

FIG. 53 shows kinetic analysis of selected scFv-Fc candidates from SARS-2 S1/RBD panning. Three rounds of panning for anti-SARS-2 S1/RBD antibodies was done using recombinantly expressed soluble protein. a large number of antibodies with varying kinetic properties. Antibodies highlighted in blue are suspected of binding S1 outside of the RBD.

FIG. 54 shows Epitope binning of anti-SARS-CoV Spike scFvFc's. Competitive binding assay was run to identify antibodies that bind different epitopes. Sensors were first saturated with Ab 1 (250 nM), then Ab 2 (125 nM) was added. If there was additional antibody binding as demonstrated in the top panel, the antibodies were considered to bind separate epitopes. Results from these competition assays were compiled in a matrix as seen in the middle panel. Once the antibodies were grouped into general clusters, a more detailed competition assay was performed to further differentiate the broader bins as seen in Bin 3. Based on this analysis, the antibodies fell into 3 major bins and 7 minor bins.

FIG. 55 is a graph showing Percent pseudovirus neutralization by Anti-Spike scFvFcs from Different Bins.

FIG. 56 is a graph showing FACS Staining of Anti-Spike scFvFc to SARS2 Spike-293T cells. 100 k 293T+/−SARS2 Spike cells were stained with 100 ul of scFv-Fc at 5 ug/ml. Binding was detected by anti-human Fc APC. CR3022 is full IgG. This is selected data from FIG. 74.

FIG. 57 is a graph showing a dose-response curve for monoclonal antibody Ab-12 neutralization activity against live SARS-CoV-2 virus.

FIG. 58 is a graph showing a dose-response curve for monoclonal antibody Ab-27 neutralization activity against live SARS-CoV-2 virus.

FIG. 59 is a graph showing a dose-response curve for monoclonal antibody Ab-14 neutralization activity against live SARS-CoV-2 virus.

FIG. 60 is a graph showing a dose-response curve for monoclonal antibody Ab-19 neutralization activity against live SARS-CoV-2 virus.

FIG. 61 is a graph showing a dose-response curve for monoclonal antibody Ab-28 neutralization activity against live SARS-CoV-2 virus.

FIG. 62 is a bar graph showing anti-SARS-CoV2 scFv-Fcs pseudovirus neutralization at 100 μg/ml.

FIG. 63 is a bar graph for anti-SARS-CoV2 scFv-Fc pseudovirus neutralization dilutions.

FIG. 64 is a bar graph for pseudovirus neutralization dilution curves for anti-SARS-CoV2 scFv-FCs. Ab 14=Ab 27>Ab 19>Ab 23>Ab 26>Ab 28

FIG. 65 is a schematic showing master binning for Abs 1-28.

FIG. 66 is a schematic showing ACE2 competition. The value in the box is the percent binding normalized to the unblocked sensor. Shading was done manually since our antibodies have a range of binding characteristics and maximum, unblocked binding could have been below the threshold.

FIG. 67 is a bar graph for pseudovirus neutralization of anti-SARS-CoV2 scFv-FCs.

FIG. 68 is a line graph for pseudovirus neutralization of anti-SARS-CoV2 scFv-FCs.

FIG. 69 is an epitope binning schematic. Based on competition matrix, Abs fell into 3 major bins which were further divided into 8 subbins.

FIG. 70 is a schematic of epitope binning of Abs 29-40, repeat Abs 1-8.

FIG. 71 is a schematic of epitope binning of further competition with Ab 12 group. Competition of the antibodies in the Ab 12 group leads to some interesting sub bins. Additionally, a few of the strong binders compete with Ab 12, but do not compete with ab 27 or ACE2 (i.e. Ab 35). Based upon the results here, Ab 27 competition is more correlated with ACE2 blockade compared to Ab 12 competition.

FIG. 72 is a schematic of epitope binning of further competition with CR3022 group. All Abs in the CR3022 bind have similar competition patterns. The only difference is that our Abs appear to block ACE2 whereas CR3022 does not. CR3022 is known to bind outside of the ACE2/RBD interface.

FIG. 73 is a schematic of epitope binning of further competition with S1 binding group. Abs 5, 23, 30 all bind to the S1 outside of RBD. Interestingly all of the abs appear to target the same epitope as they compete with each other. To confirm, they do not compete with ACE2, CR3022, Ab 12, or Ab 27.

FIG. 74 is a plot depicting FACS binding of scFv-FCs to 293T+/−SARS2 spike. FACS binding at single concentration (5 ug/ml) of scFv-Fc with transduced 293T-SARS2-Spike expressing cells. Cells were first gated for BFP (transduced cells) and then for antibody binding. Some of the background may be due to the inherent stickiness of scFv-Fcs. Binding was detected with anti-human-Fc APC from Biolegend.

FIG. 75 is a binding curve showing Ab-12 binding to SARS-2 spike expressing cells.

FIG. 76 is a binding curve showing Ab-12 binding to 293T cells. 293T cells were transduced with SARS-2 lentivirus. FACS was done with cells before sorting. Only BFP+ cells were used in the analysis of Ab binding. Untransduced 293T cells were used as the negative. IgG and scFv-Fc were detected by anti-human-Fc-APC and the Fab was detected by anti-His APC.

FIG. 77 are graphs of neutralization studies of the live SARS-CoV-2 virus.

FIG. 78 is a graph of neutralization studies of the live SARS-CoV-2 virus with mAb12 as an IgG or scFv-Fc.

FIG. 79 is a graph of neutralization studies of the live SARS-CoV-2 virus with mAb14 as an IgG or scFv-Fc.

FIG. 80 is a graph of neutralization studies of the live SARS-CoV-2 virus with mAb27. Note: Ab 27 IgG was actually Ab 2-2 IgG, for 27 data see FIG. 146.

FIG. 81 is a graph of neutralization studies of the live SARS-CoV-2 virus with mAb27.

FIG. 82 is a graph of neutralization studies of the live SARS-CoV-2 virus with mAb29 as an IgG or scFv-Fc.

FIG. 83 is a graph of neutralization studies of the live SARS-CoV-2 virus with mAb2-7 as an IgG or scFv-Fc.

FIG. 84 is a graph of neutralization studies of the live SARS-CoV-2 virus with mAb38 as an IgG or scFv-Fc.

FIG. 85 is a graph of neutralization studies of the live SARS-CoV-2 virus with mAb5 as an IgG or scFv-Fc.

FIG. 86 is a graph of neutralization studies of the live SARS-CoV-2 virus with PD-1 control as an IgG or scFv-Fc.

FIG. 87 shows graphs of neutralization studies of the live SARS-CoV-2 virus engineered to express luciferase. Neutralization was also tested with a mouse adapted variant of SARS-CoV-2 (Dinnon, K. H., Leist, S. R., Schafer, A. et al. A mouse-adapted model of SARS-CoV-2 to test COVID-19 countermeasures. Nature 586, 560-566 (2020). https://doi-org.ezp-prodl.hul.harvard.edu/10.1038/s41586-020-2708-8). Mixtures of various scFv-Fcs were tested: Ab 12+Ab 2-7 (mix 1), Ab 27+Ab 2-7 (mix 2), Ab 12+Ab 27 (mix 3), Ab 27+Ab 35 (mix 4), Ab 27+Ab 2-7+Ab 30 (mix 5).

FIG. 88 shows graphs of neutralization studies of the live SARS-CoV-2 virus engineered to express luciferase.

FIG. 89 shows graphs of neutralization studies of the live SARS-CoV-2 virus comparing WT and D614G mutants.

FIG. 90 shows graphs of weight loss in hamsters (TOP) and Viral load of lung tissues, 3 dpi, PFU/g (BOTTOM) of hamsters. Therapeutic treatment of Syrian golden hamsters post infection with Ab 12 IgG or Ab 2-7 scFv-Fc leads to a 513.9- and 5-fold reduction respectively compared to control treated animals. No significant difference in weight loss is observed.

FIG. 91 shows graphs of serum neutralization, day 3 post infection in hamsters. Serum was collected 3 days post infection and tested in vitro neutralization assays. Serum from Ab 12 treated animals is able to neutralize virus, whereas serum from Ab 2-7 and control treated animals is not.

FIG. 92 shows images of lung pathology studies and a graph depicting gross lesions Score, 3 dpi.

FIG. 93 shows images of lung pathology studies and a graph depicting gross lesions score, 3 dpi.

FIG. 94 are graphs showing lung lesion scores in hamsters treated with mAb 12.

FIG. 95 shows a graph a of serum neutralization study.

FIG. 96 are graphs showing lung lesion scores in hamsters treated with mAb 12.

FIG. 97 are fluorescent micrographs showing the visualization of antibody dependent enhancement SARS CoV-2.

FIG. 98 shows competition of the antibodies in the Ab 12 group. For example, a few of the strong binders compete with Ab 12, but do not compete with Ab 27 or ACE2 (i.e. Ab 35). Ab 27 competition is more correlated with ACE2 blockade compared to Ab 12 competition.

FIG. 99 shows further competition within CR3022 group. All Abs in the CR3022 bind have similar competition patterns. The only difference is that the Abs appear to block ACE2 whereas CR3022 does not. CR3022 is known to bind outside of the ACE2/RBD interface.

FIG. 100 shows further competition within S1 binding group. Abs 5, 23, 30 all bind to the S1 outside of RBD. Without wishing to be bound by theory, the antibodies target the same epitope as they compete with each other. To confirm, they do not compete with ACE2, CR3022, Ab 12, or Ab 27.

FIG. 101 is a schematic of epitope binning. Bin 1: S1, non RBD binding; Bin 2: RBD binding, competes with CR3022; Bin 3: RBD binding, non CR3022 competition.

FIG. 102 is a graph showing SARS-CoV-2 virus neutralization by scFv-Fc.

FIG. 103 shows graphs of IgG vs scFv-Fc virus neutralization. scFv-Fcs and IgGs were tested in parallel SARS-CoV-2 neutralization assays. Abs 12 and 38 showed minimal loss in neutralization efficacy, however Abs 14, 27, 29, and 2-7 displayed substantial loss. Of the 3 most potent scFv-Fcs, Ab 12 is an Ab that maintains its ability to neutralize as an IgG, while the IC50 for Ab 14 and Ab 27 shifts the right by 40- and 950-fold, respectively.

FIG. 104 shows FACS binding curves with 293T-Spike cells show a pronounced decrease in binding for Abs 14, 27, and 2-7 whereas Ab 12 shows an increase in binding. Kinetic measurements via BLI of selected Abs show that conversion from scFv to IgG does not have a significant effect on the KD values for these antibodies when binding.

FIG. 105 shows graphs of pathology scores for animals treated with PBS or anti-SARS-CoV2 Abs 12 or 2-7. *=p<0.05; ns=Not significant (Kruskall-Wallis test with Dunn's post-hoc correction)

FIG. 106 shows lung histology images. A) and B) are not depicted in this image. C) Control lung. Extensive consolidation with multiple foci of inflammatory infiltration. Magnified images (locations on low magnification images marked with numbers): (1) Airways are obstructed by inflammatory cells (combination of MNC and PMNs). (2) Airway epithelial hyperplasia notable. Perivascular cuffing and congestion prominent. D) Ab 2-7 lung. Extensive consolidation with multiple foci of inflammatory infiltration. (1) Pleuritis noted, but less severe. (2) Fewer inflammatory cells in airways, but hyperplasia of airway epithelia is still prominent. E) Ab 12 lung. Consolidation markedly reduced, with fewer and smaller foci of inflammatory infiltration. Infiltrating inflammatory cells present in some airways. (1) Pleuritis is moderate relative to control. (2) Airway epithelial hypertrophy still present.

FIG. 107 is a graph showing Spike shedding induced by scFv-Fcs. FACS based Spike shedding experiment comparing parental Abs with the BsAbs. A decrease in median fluorescence correlates to an increase in spike shedding whereas an increase in fluorescence indicates minimal shedding is observed. Antibodies that are grouped into a similar sub-bin show similar levels of shedding. Western blot image is also shown depicting the detection shed S1 in supernatant from Ab 12 IgG spike shedding experiment confirming decreasing fluorescence is a result of shedding and not internalization of the spike-Ab complex.

FIG. 108 is a cryo-EM image of Ab 5, starting at medium resolution. Without wishing to be bound by theory, Ab 5 is an anti-NTD binder.

FIG. 109 are cryo-EM images of Ab 38: 2D classification.

FIG. 110 shows cryo-EM images of Ab 12, at Medium resolution (5 Å) to begin. Without wishing to be by theory, the red arrow in the bottom figure points to a quaternary epitope: Glycan N165 from a different monomer can be involved in the epitope.

FIG. 111 shows a map refined to a nominal resolution of 2.97 Angstroms.

FIG. 112 shows a schematic of the refinement of a mixed population.

FIG. 113 shows images of cryoEM of the scFv-bound species and the map in the region of the RBD/scFv.

FIG. 114 shows images of a cryoEM map depicting three scFv molecules bound to a spike trimer, with 3-fold symmetry.

FIG. 115 shows images of a cryoEM map depicting a mixed interaction between heavy chain and light chain.

FIG. 116 shows cryoEM images of a further refined Ab2-7. Spike is blue, heavy chain is orange, light chain is gray.

FIG. 117 shows broad epitope binding for whole cell panning derived phage. Phage supernatant of unique clones were tested via ELISA against the different SARS-CoV-2 subunits (S1, S2, RBD) and against full length spikes from SARS-CoV-2 (D614G) and SARS-CoV. IL2-Fc was used as a negative control. Values shown are OD450 values, phage binding is detected with anti-M13-HRP. As shown here, a number of phage bind to the full length spike but not to any of the individually expressed domains. Without wishing to be bound by theory, this can be due to a conformational shift or junction epitope. These clones do not appear to non-specifically bind to the plates as the IL2 signal is negligible. One antibody appears to cross react between SARS1 and SARS2 and it binds the S2 domain, which generally is more conserved than the S1 domains.

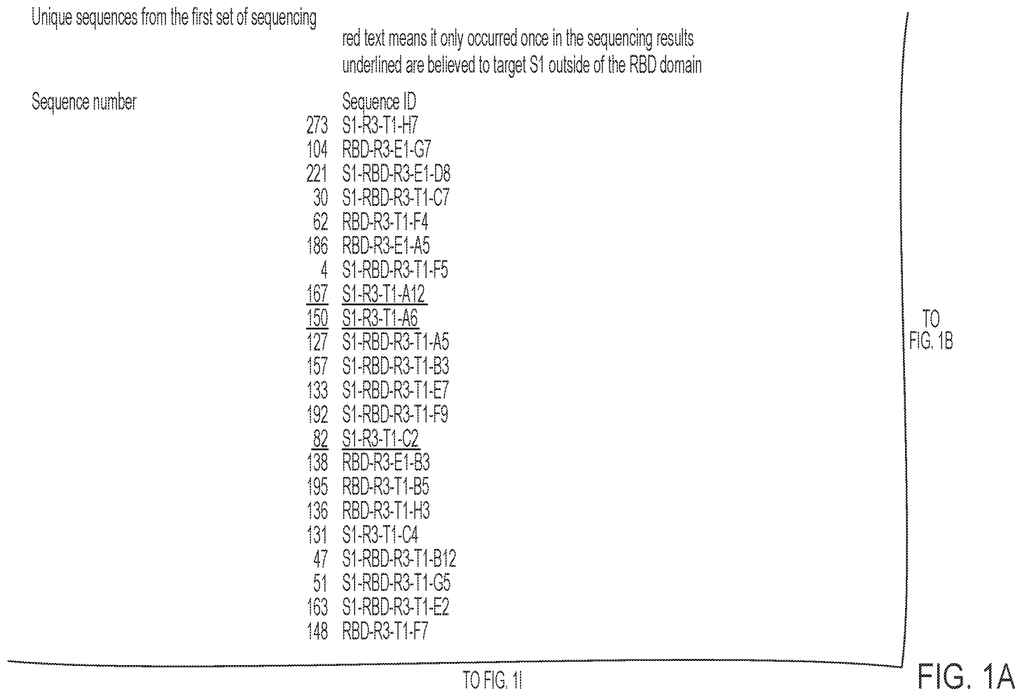

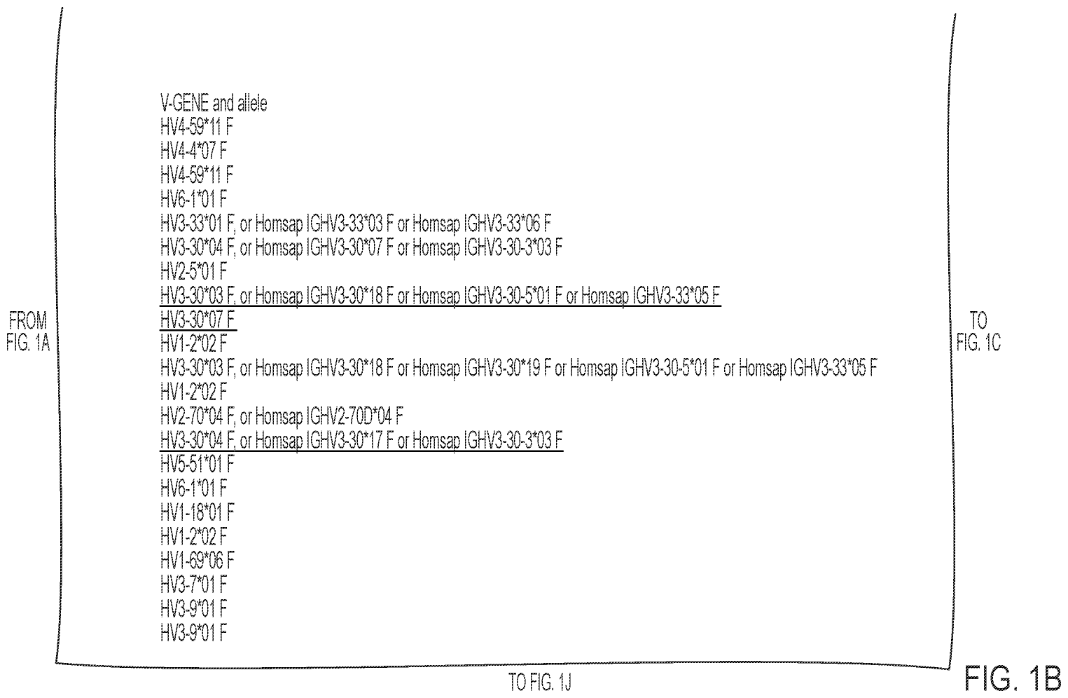

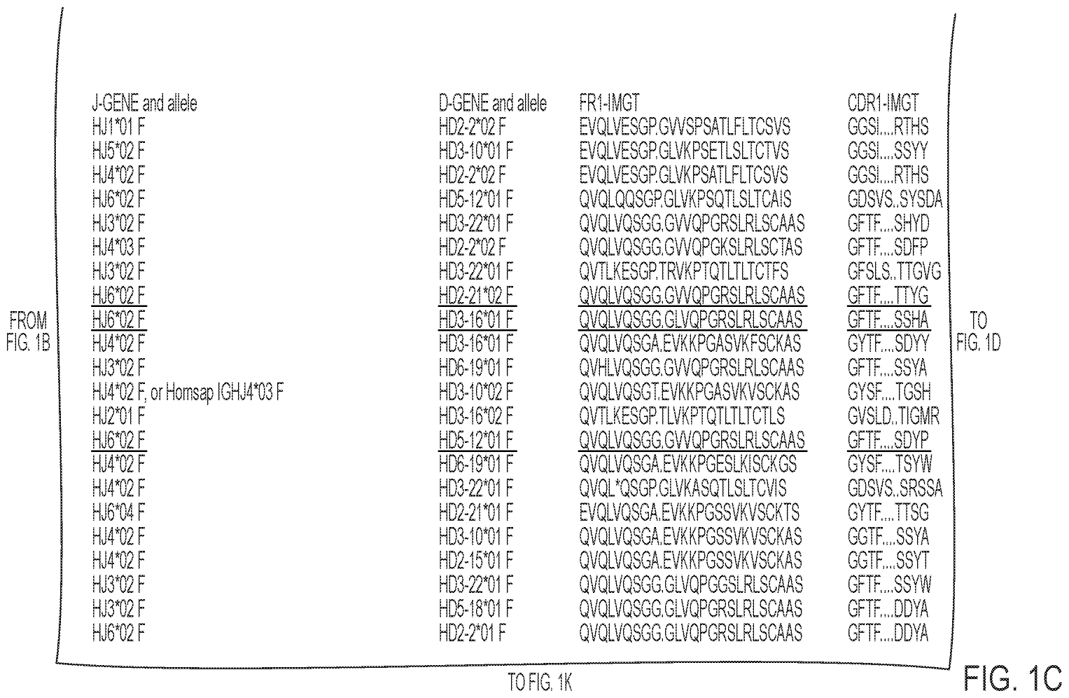

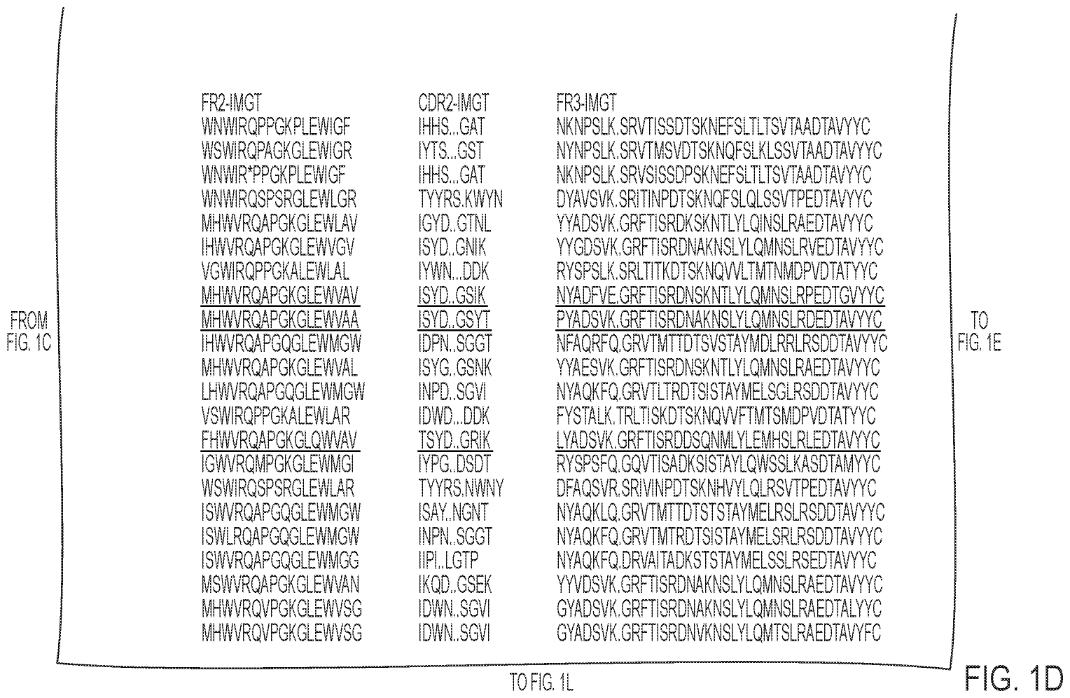

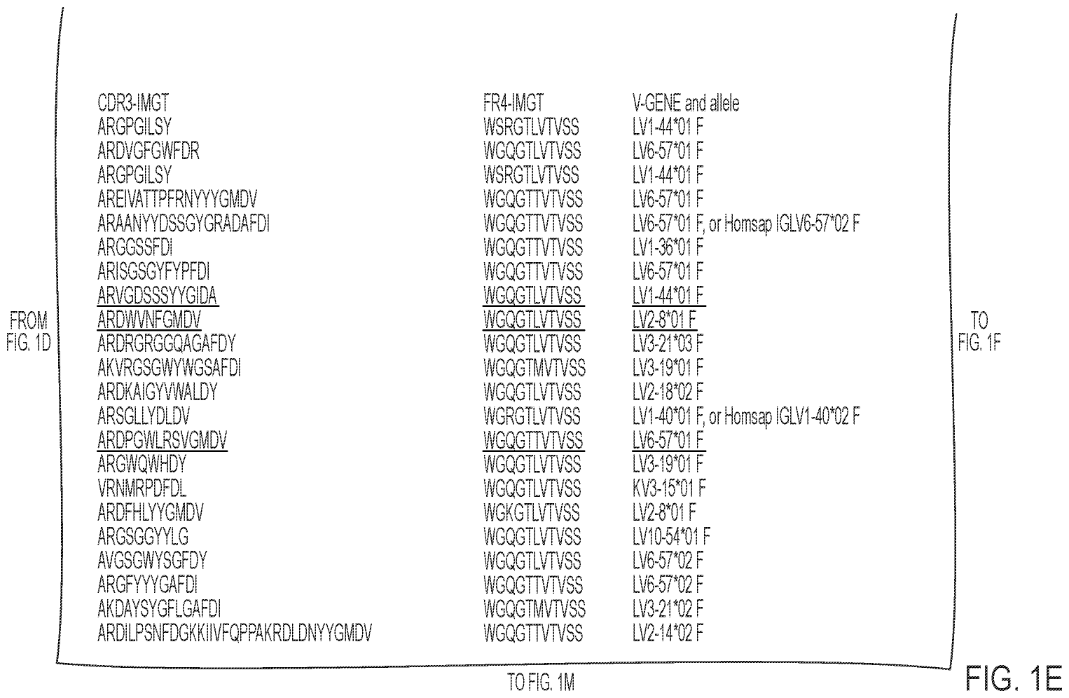

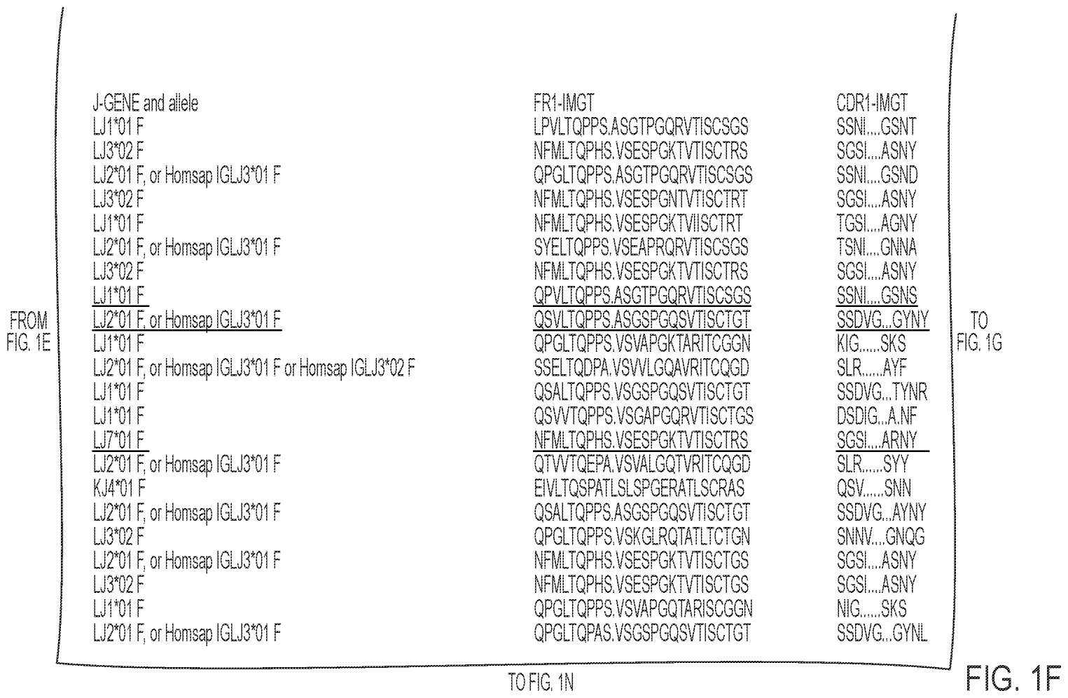

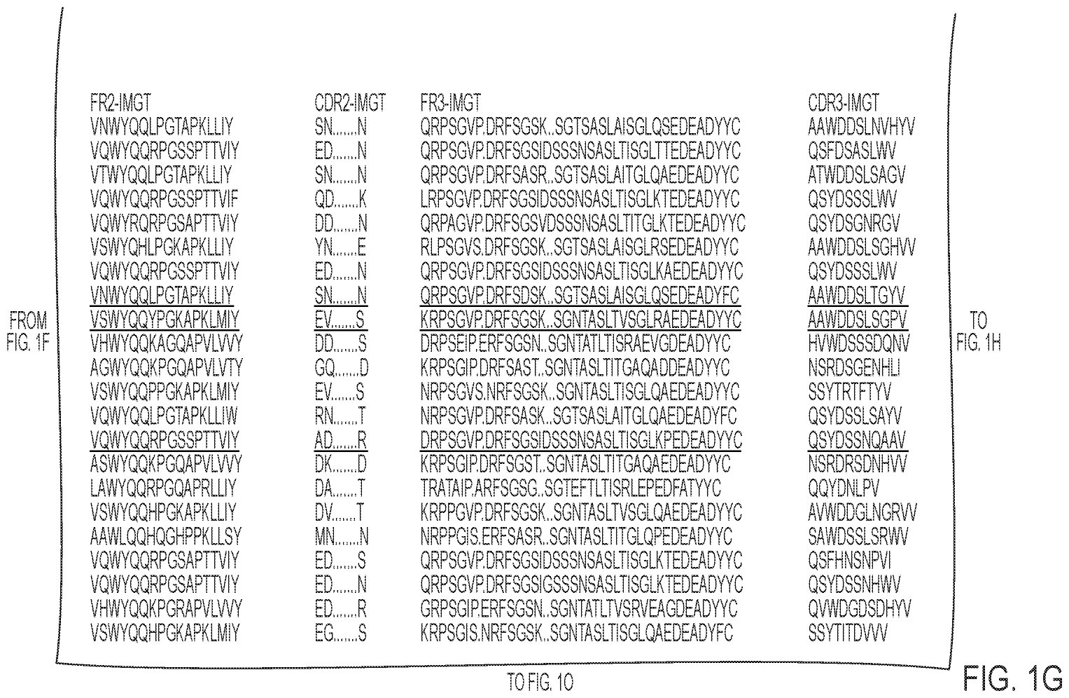

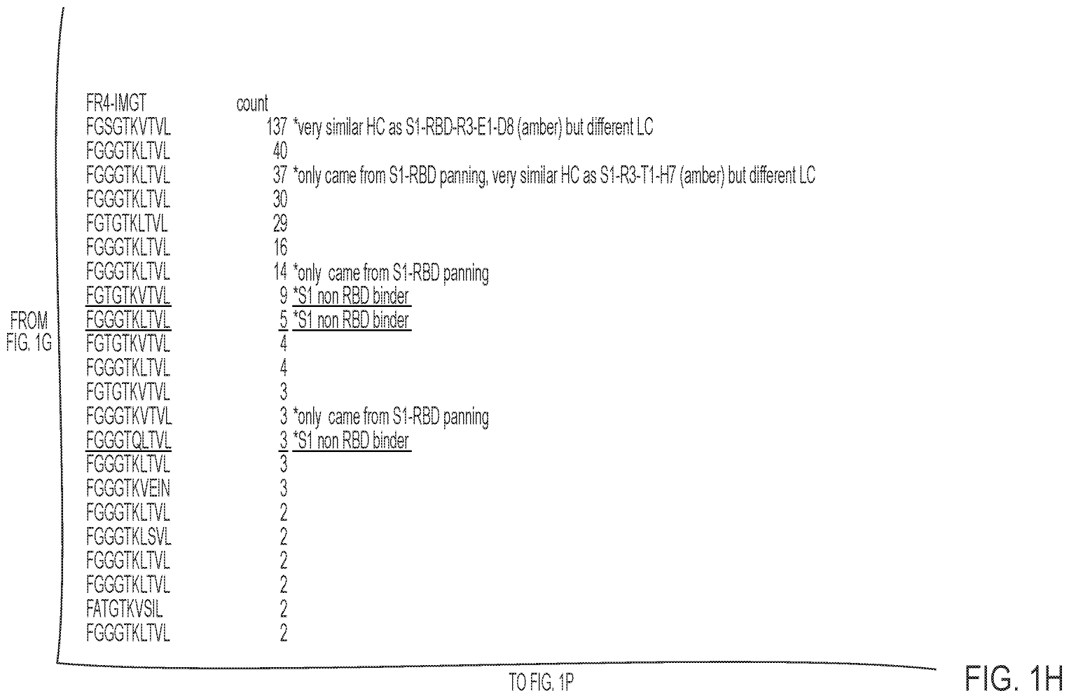

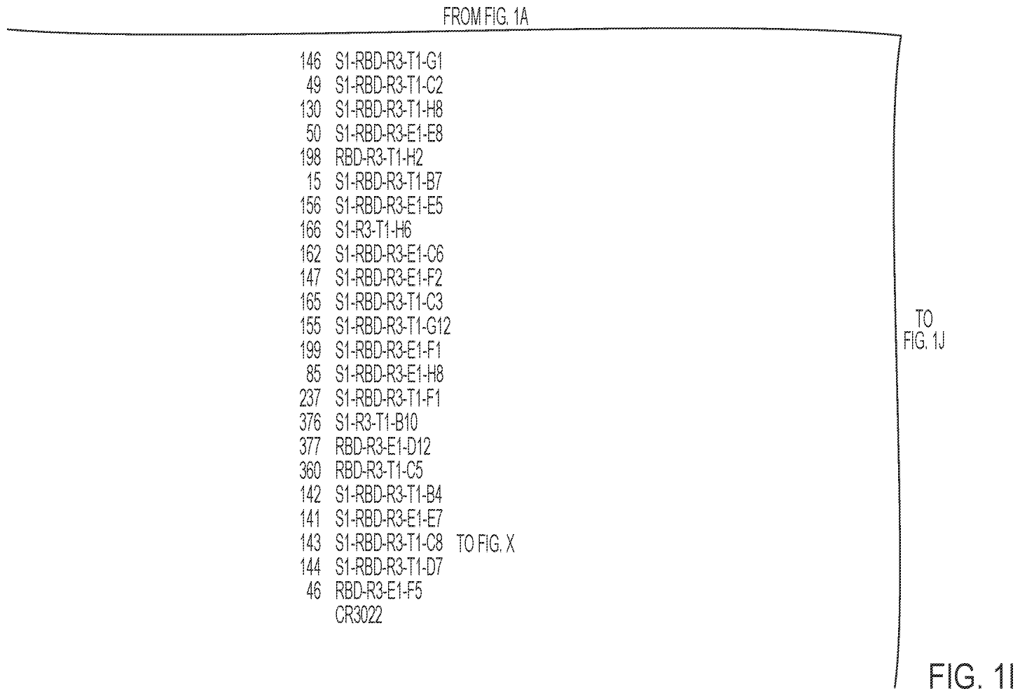

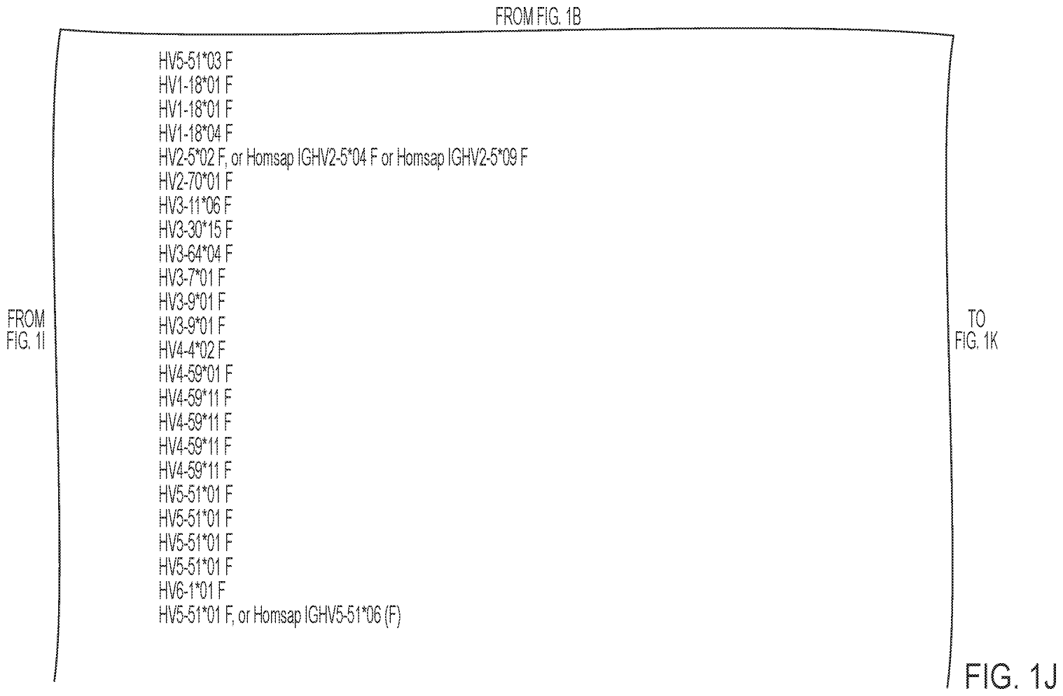

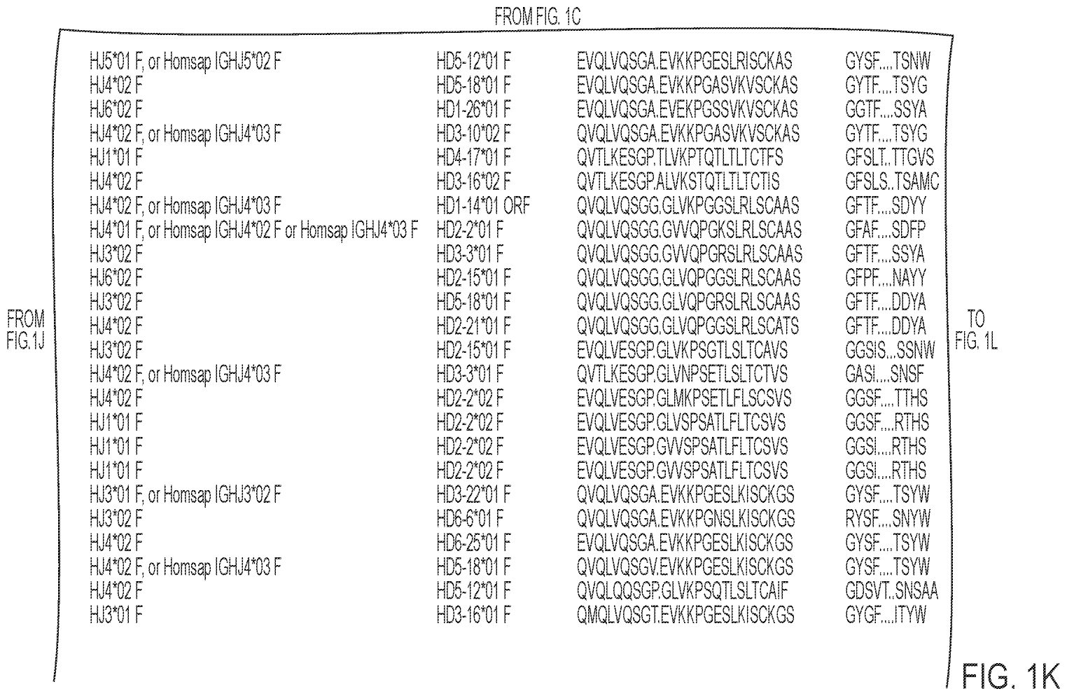

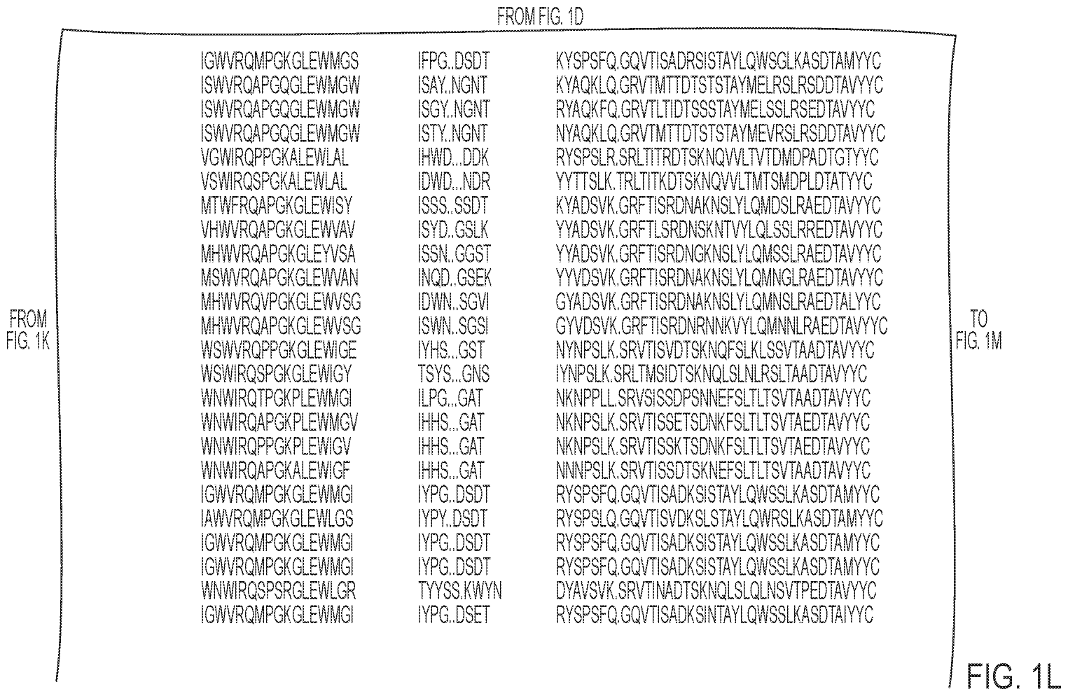

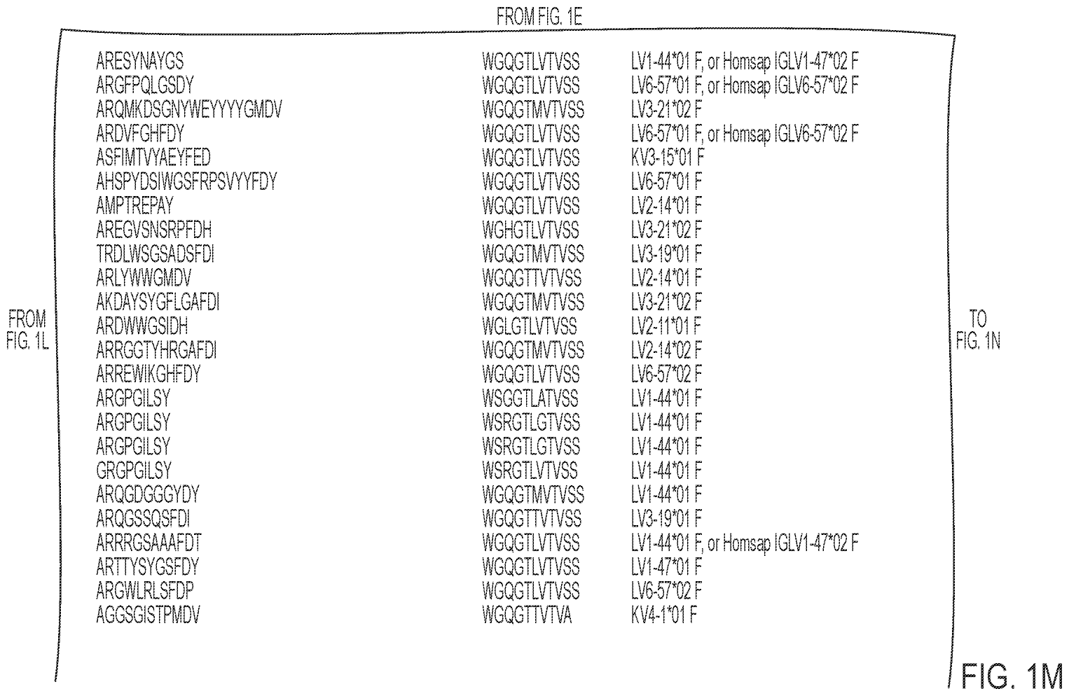

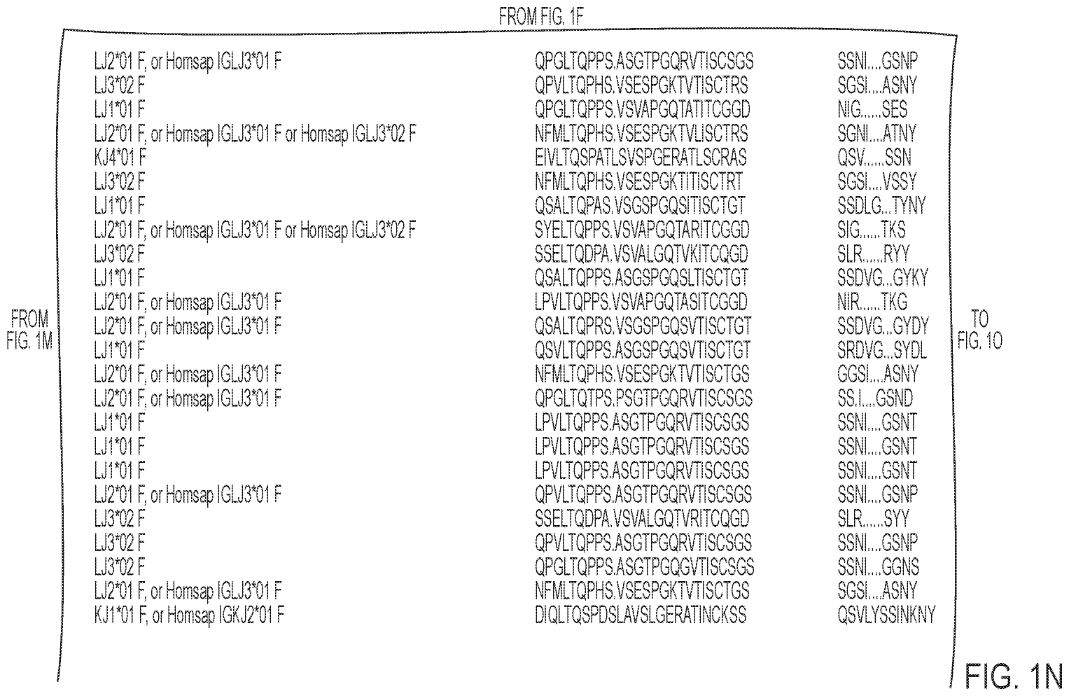

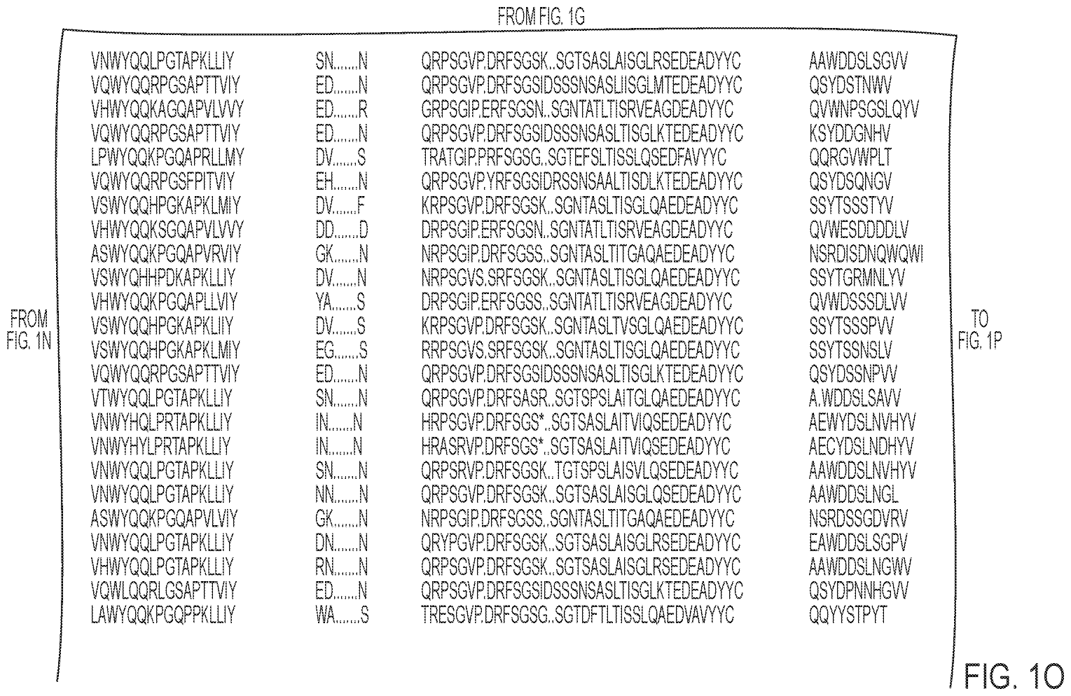

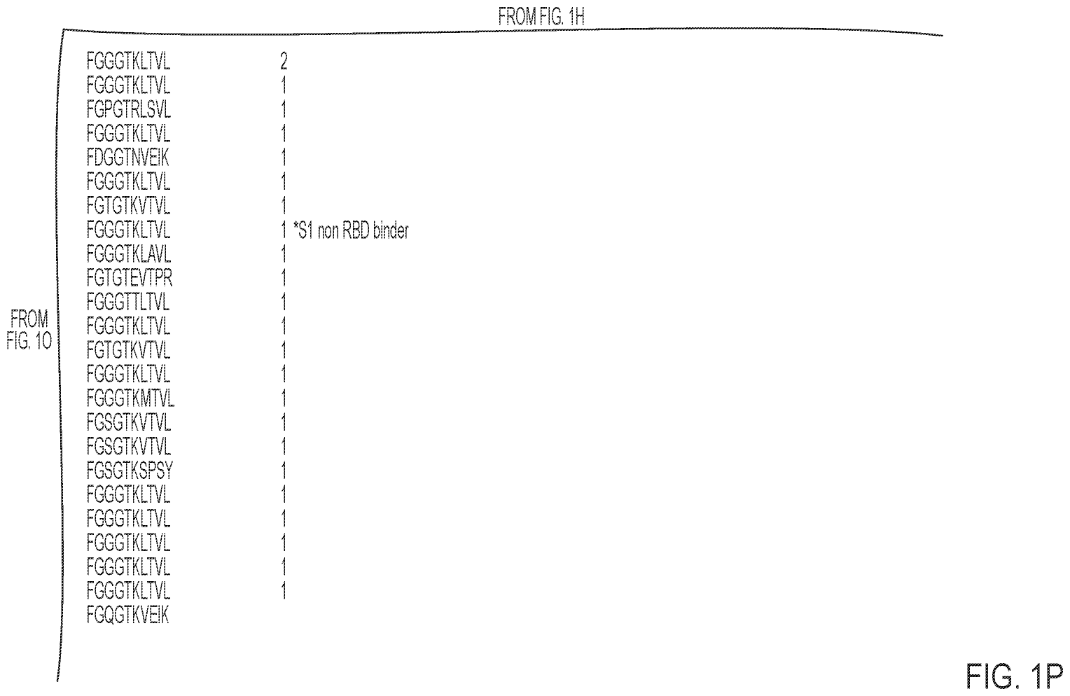

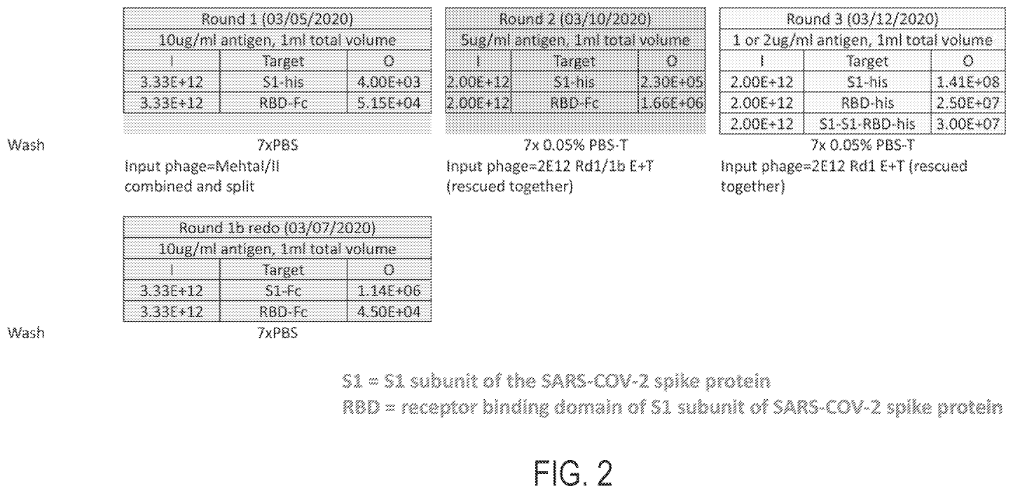

### DETAILED DESCRIPTION

The present invention provides antibodies that are directed to severe acute respiratory syndrome-associated coronavirus (SARS-CoV2). In some embodiments, the antibodies described herein can neutralize infection by severe acute respiratory syndrome-associated coronavirus (SARS-CoV2). In other embodiments, the SARS-CoV2 antibodies, for example non-neutralizing antibodies, can be useful for diagnostic purposes. Specifically, anti-SARS-CoV2 Abs were isolated from a non-immune human Ab-phage library using a panning strategy.

The amino acid sequence of the monoclonal SARS-CoV2 antibodies are provided below; the amino acid sequences of the heavy and light chain complementary determining regions CDRs of the COVID-19 antibodies are underlined (CDR1), underlined and bolded (CDR2), or [symbol] [symbol] below:

TABLE 1
S1-R3-T1-H7 (Ab12) Ab Variable
Region amino acid sequences

V_{H} chain of H7
EVQLVESGPGVVSPSATLFLTCSVSGGSIRTHSWNWIRQP
PGKPLEWIGFIHHSGATNKNPSLKSRVTISSDTSKNEFSL
TLTSVTAADTAVYYCARGPGILSYWSRGTLVTVSS
(SEQ ID NO: 1)
V_{L} chain of H7
LPVLTQPPSASGTPGQRVTISCSGSSSNIGSNTVNWYQQL
PGTAPKLLIYSNNQRPSGVP.DRFSGSKSGTSASLAISGL
QSEDEADYYCAAWDDSLNVHYVFGSGTKVTVL
(SEQ ID NO: 2)

TABLE 2
RBD-R3-E1-G7 (Ab2-10) Ab Variable
Region amino acid sequences

V_{H} chain of G7
EVQLVESGPGLVKPSETLSLTCTVSGGSISSYYWSWIRQP
AGKGLEWIGRIYTSGSTNYNPSLKSRVTMSVDTSKNQFSL
KLSSVTAADTAVYYCARDVGFGWFDRWGQGTLVTVSS
(SEQ ID NO: 3)
V_{L} chain of G7
NFMLTQPHSVSESPGKTVTISCTRSSGSIASNYVQWYQQR
PGSSPTTVIYEDNQRPSGVPDRFSGSIDSSSNSASLTISG
LTTEDEADYYCQSFDSASLWVFGGGTKLTVL
(SEQ ID NO: 4)

TABLE 3
S1-RBD-R3-E1-D8 (Ab_3) Ab Variable
Region amino acid sequences

V_{H} chain of D8
EVQLVESGPGLVKPSATLFLTCSVSGGSIRTHSWNWIR*P
PGKPLEWIGFIHHSGATNKNPSLKSRVSISSDPSKNEFSL
TLTSVTAADTAVYYCARGPGILSYWSRGTLVTVSS
SEQ ID NO: 5)
V_{L} chain of D8
QPGLTQPPSASGTPGQRVTISCSGSSSNIGSNDVTWYQQL
PGTAPKLLIYSNNQRPSGVPDRFSASRSGTSASLAITGLQ
AEDEADYYCATWDDSLSAGVFGGGTKLTVL
(SEQ ID NO: 6)

TABLE 4
S1-RBD-R3-T1-C7 Ab (Ab 29) Variable
Region amino acid sequences

V_{H} chain of C7
QVQLQQSGPGLVKPSQTLSLTCAISGDSVSSYSDAWNWIR
QSPSRGLEWLGRTYYRSKWYNDYAVSVKSRITINPDTSKN
QFSLQLSSVTPEDTAVYYCAREIVATTPFRNYYYGMDVWG
QGTTVTVSS
(SEQ ID NO: 7)
V_{L} chain of C7
NFMLTQPHSVSESPGNTVTISCTRTSGSIASNYVQWYQQR
PGSSPTTVIFQDKLRPSGVPDRFSGSIDSSSNSASLTISG
LKTEDEADYYCQSYDSSSLWVFGGGTKLTVL
(SEQ ID NO: 8)

TABLE 5
RBD-R3-T1-F4 (Ab2-2) Ab Variable
Region amino acid sequences

V_{H} chain of F4
QVQLVQSGGGVVQPGRSLRLSCAASGFTFSHYDMHWVRQA
PGKGLEWLAVIGYDGTNLYYADSVKGRFTISRDKSKNTLY
LQINSLRAEDTAVYYCARAANYYDSSGYGRADAFDIWGQG
TTVTVSS
(SEQ ID NO: 9)
V_{L} chain of F4
NFMLTQPHSVSESPGKTVIISCTRTTGSIAGNYVQWYRQR
PGSAPTTVIYDDNQRPAGVPDRFSGSVDSSSNSASLTITG
LKTEDEADYYCQSYDSGNRGVFGTGTKLTVL
(SEQ ID NO: 10)

TABLE 6
RBD-R3-E1-A5 Ab (Ab_30) Variable
Region amino acid sequences

V_{H} chain of A5
QVQLVQSGGGVVQPGKSLRLSCTASGFTFSDFPIHWVRQA
PGKGLEWVGVISYDGNIKYYGDSVKGRFTISRDNAKNSLY
LQMNSLRVEDTAVYYCARGGSSFDIWGQGTTVTVSS
(SEQ ID NO: 11)
V_{L} chain of A5
SYELTQPPSVSEAPRQRVTISCSGSTSNIGNNAVSWYQHL
PGKAPKLLIYYNERLPSGVSDRFSGSKSGTSASLAISGLR
SEDEADYYCAAWDDSLSGHVVFGGGTKLTVL
(SEQ ID NO: 12)

TABLE 7
S1-RBD-R3-T1-F5 (Ab2-7) Ab Variable
Region amino acid sequences

V_{H} chain of F5
QVTLKESGPTRVKPTQTLTLTCTFSGFSLSTTGVGVGWIR
QPPGKALEWLALIYWNDDKRYSPSLKSRLTITKDTSKNQV
VLTMTNMDPVDTATYYCARISGSGYFYPFDIWGQGTTVTV
SS
SEQ ID NO: 13)
V_{L} chain of F5
NFMLTQPHSVSESPGKTVTISCTRSSGSIASNYVQWYQQR
PGSSPTTVIYEDNQRPSGVPDRFSGSIDSSSNSASLTISG
LKAEDEADYYCQSYDSSSLWVFGGGTKLTVL
(SEQ ID NO: 14)

TABLE 8
S1-R3-T1-A12 (Ab_5) Ab Variable Region
amino acid sequences

V_{H} chain of A12
QVQLVQSGGGVVQPGRSLRLSCAASGFTFTTYGMHWVRQAPGK
GLEWVAVISYDGSIKNYADFVEGRFTISRDNSKNTLYLQMNSL
RPEDTGVYYCARVGDSSSYYGIDAWGQGTLVTVSS
(SEQ ID NO: 15)
V_{L} chain of A12
QPVLTQPPSASGTPGQRVTISCSGSSSNIGSNSVNWYQQLPGT
APKLLIYSNNQRPSGVPDRFSDSKSGTSASLAISGLQSEDEAD
YFCAAWDDSLTGYVFGTGTKVTVL
(SEQ ID NO: 16)

TABLE 9
S1-R3-T1-A6 (Ab_4) Ab Variable
Region amino acid sequences

V_{H} chain of A6
QVQLVQSGGGLVQPGRSLRLSCAASGFTFSSHAMHWVRQAPGKG
LEWVAAISYDGSYTPYADSVKGRFTISRDNAKNSLYLQMNSLRD
EDTAVYYCARDWVNFGMDVWGQGTLVTVSS
(SEQ ID NO: 17)
V_{L} chain of A6
QSVLTQPPSASGSPGQSVTISCTGTSSDVGGYNYVSWYQQYPGK
APKLMIYEVSKRPSGVPDRFSGSKSGNTASLTVSGLRAEDEADY
YCAAWDDSLSGPVFGGGTKLTVL
(SEQ ID NO: 18)

TABLE 10
S1-RBD-R3-T1-A5 Ab (Ab_33) Variable
Region amino acid sequences

V_{H} chain of T1-A5
QVQLVQSGAEVKKPGASVKFSCKASGYTFSDYYIHWVRQAPGQ
GLEWMGWIDPNSGGTNFAQRFQGRVTMTTDTSVSTAYMDLRRL
RSDDTAVYYCARDRGRGGQAGAFDYWGQGTLVTVSS
(SEQ ID NO: 19)
V_{L} chain of T1-A5
QPGLTQPPSVSVAPGKTARITCGGNKIGSKSVHWYQQKAGQAP
VLVVYDDSDRPSEIPERFSGSNSGNTATLTISRAEVGDEADYY
CHVWDSSSDQNVFGTGTKVTVL
(SEQ ID NO: 20)

TABLE 11
S1-RBD-R3-T1-B3 (Ab_1) Ab Variable
Region amino acid sequences

V_{H} chain of B3
QVHLVQSGGGVVQPGRSLRLSCAASGFTFSSYAMHWVRQAPGKG
LEWVALISYGGSNKYYAESVKGRFTISRDNSKNTLYLQMNSLRA
EDTAVYYCAKVRGSGWYWGSAFDIWGQGTMVTVSS
(SEQ ID NO: 21)
V_{L} chain of B3
SSELTQDPAVSVVLGQAVRITCQGDSLRAYFAGWYQQKPGQAPV
LVTYGQDKRPSGIPDRFSASTSGNTASLTITGAQADDEADYYCN
SRDSGENHLIFGGGTKLTVL
(SEQ ID NO: 22)

TABLE 12
S1-RBD-R3-T1-E7 Ab Variable
Region amino acid sequences

V_{H} chain of E7
QVQLVQSGTEVKKPGASVKVSCKASGYSFTGSHLHWVRQAPG
QGLEWMGWINPDSGVINYAQKFQGRVTLTRDTSISTAYMELS
GLRSDDTAVYYCARDKAIGYVWALDYWGQGTLVTVSS
(SEQ ID NO: 23)
V_{L} chain of E7
QSALTQPPSVSGSPGQSVTISCTGTSSDVGTYNRVSWYQQPP
GKAPKLMIYEVSNRPSGVSNRFSGSKSGNTASLTISGLQAED
EADYYCSSYTRTFTYVFGTGTKVTVL
(SEQ ID NO: 24)

TABLE 13
S1-RBD-R3-T1-F9 Ab (Ab_39) Variable
Region amino acid sequences

V_{H} chain of F9
QVTLKESGPTLVKPTQTLTLTCTLSGVSLDTIGMRVSWIRQPPG
KALEWLARIDWDDDKFYSTALKTRLTISKDTSKNQVVFTMTSMD
PVDTATYYCARSGLLYDLDVWGRGTLVTVSS
(SEQ ID NO: 25)
V_{L} chain of F9
QSVVTQPPSVSGAPGQRVTISCTGSDSDIGANFVQWYQQLPGTA
PKLLIWRNTNRPSGVPDRFSASKSGTSASLAITGLQAEDEADYF
CQSYDSSLSAYVFGGGTKVTVL
(SEQ ID NO: 26)

TABLE 14
S1-R3-T1-C2 (Ab_6) Ab Variable Region
amino acid sequences

V_{H} chain of C2
QVQLVQSGGGVVQPGRSLRLSCAASGFTFSDYPFHWVRQAPGK
GLQWVAVTSYDGRIKLYADSVKGRFTISRDDSQNMLYLEMHSL
RLEDTAVYYCARDPGWLRSVGMDVWGQGTTVTVSS
SEQ ID NO: 27)
V_{L} chain of C2
NFMLTQPHSVSESPGKTVTISCTRSSGSIARNYVQWYQQRPGSS
PTTVIYADRDRPSGVPDRFSGSIDSSSNSASLTISGLKPEDEAD
YYCQSYDSSNQAAVFGGGTQLTVL
(SEQ ID NO: 28)

TABLE 15
RBD-R3-E1-B3 Ab (Ab_32) Variable
Region amino acid sequences

V_{H} chain of E1-B3
QVQLVQSGAEVKKPGESLKISCKGSGYSFTSYWIGWVRQMPGK
GLEWMGIIYPGDSDTRYSPSFQGQVTISADKSISTAYLQWSSL
KASDTAMYYCARGWQWHDYWGQGTLVTVSS
(SEQ ID NO: 29)
V_{L} chain of E1-B3
QTVVTQEPAVSVALGQTVRITCQGDSLRSYYASWYQQKPGQAP
VLVVYDKDKRPSGIPDRFSGSTSGNTASLTITGAQAEDEADYY
CNSRDRSDNHVVFGGGTKLTVL
(SEQ ID NO: 30)

TABLE 16
RBD-R3-T1-B5 Ab Variable Region
amino acid sequences

V_{H} chain of B5
QVQL=QSGPGLVKASQTLSLTCVISGDSVSSRSSAWSWIRQSPS
RGLEWLARTYYRSNWNYDFAQSVRSRIVINPDTSKNHVYLQLRS
VTPEDTAVYYCVRNMRPDFDLWGQGTLVTVSS
(SEQ ID NO: 31)
V_{L} chain of B5
EIVLTQSPATLSLSPGERATLSCRASQSVSNNLAWYQQRPGQAP
RLLIYDATTRATAIPARFSGSGSGTEFTLTISRLEPEDFATYYC
QQYDNLPVFGGGTKVEIN
(SEQ ID NO: 32)

TABLE 17
RBD-R3-T1-H3 (Ab_2) Ab Variable
Region amino acid sequences

V_{H} chain of H3
EVQLVQSGAEVKKPGSSVKVSCKTSGYTFTTSGISWVRQAPGQ
GLEWMGWISAYNGNTNYAQKLQGRVTMTTDTSTSTAYMELRSL
RSDDTAVYYCARDFHLYYGMDVWGKGTLVTVSS
(SEQ ID NO: 33)
V_{L} chain of H3
QSALTQPPSASGSPGQSVTISCTGTSSDVGAYNYVSWYQQHPG
KAPKLLIYDVTKRPPGVPDRFSGSKSGNTASLTVSGLQAEDEA
DYYCAVWDDGLNGRVVFGGGTKLTVL
(SEQ ID NO: 34)

TABLE 18
S1-R3-T1-C4 (Ab_8) Ab Variable Region
amino acid sequences

V_{H} chain of C4
QVQLVQSGAEVKKPGSSVKVSCKASGGTFSSYAISWLRQAPGQG
LEWMGWINPNSGGTNYAQKFQGRVTMTRDTSISTAYMELSRLRS
DDTAVYYCARGSGGYYLGWGQGTLVTVSS
(SEQ ID NO: 35)
V_{L} chain of C4
QPGLTQPPSVSKGLRQTATLTCTGNSNNVGNQGAAWLQQHQGHP
PKLLSYMNNNRPPGISERFSASRSGNTASLTITGLQPEDEADYY
CSAWDSSLSRWVFGGGTKLSV_{L} (SEQ ID NO: 36)

TABLE 19
S1-RBD-R3-T1-B12 Ab (Ab_31) Variable Region
amino acid sequences

V_{H} chain of B12
QVQLVQSGAEVKKPGSSVKVSCKASGGTFSSYTISWVRQAPGQ
GLEWMGGIIPILGTPNYAQKFQDRVAITADKSTSTAYMELSSL
RSEDTAVYYCAVGSGWYSGFDYWGQGTLVTVSS
(SEQ ID NO: 37)
V_{L} chain of B12
NFMLTQPHSVSESPGKTVTISCTGSSGSIASNYVQWYQQRPGS
APTTVIYEDSQRPSGVPDRFSGSIDSSSNSASLTISGLKTEDE
ADYYCQSFHNSNPVTFGGGTKLTVL
(SEQ ID NO: 38)

TABLE 20
S1-RBD-R3-T1-G5 Ab Variable Region
amino acid sequences

V_{H} chain of G5
QVQLVQSGGGLVQPGGSLRLSCAASGFTFSSYWMSWVRQAPG
KGLEWVANIKQDGSEKYYVDSVKGRFTISRDNAKNSLYLQMN
SLRAEDTAVYYCARGFYYYGAFDIWGQGTTVTVSS
(SEQ ID NO: 39)
V_{L} chain of G5
NFMLTQPHSVSESPGKTVTISCTGSSGSIASNYVQWYQQRPG
SAPTTVIYEDNQRPSGVPDRFSGSIGSSSNSASLTISGLKTE
DEADYYCQSYDSSNHWVFGGGTKLTVL
(SEQ ID NO: 40)

TABLE 21
S1-RBD-R3-T1-E2 (Ab7) Ab Variable Region
amino acid sequences

V_{H} chain of E2
QVQLVQSGGGLVQPGRSLRLSCAASGFTFDDYAMHWVRQVPGKG
LEWVSGIDWNSGVIGYADSVKGRFTISRDNAKNSLYLQMNSLRA
EDTALYYCAKDAYSYGFLGAFDIWGQGTMVTVSS
SEQ ID NO: 41)
V_{L} chain of E2
QPGLTQPPSVSVAPGQTARISCGGNNIGSKSVHWYQQKPGRAPV
LVVYEDRGRPSGIPERFSGSNSGNTATLTVSRVEAGDEADYYCQ
VWDGDSDHYVFATGTKVSIL
SEQ ID NO: 42)

TABLE 22
RBD-R3-T1-F7 Ab Variable Region amino
acid sequences

V_{H} chain of F7
QVQLVQSGGGLVQPGRSLRLSCAASGFTFDDYAMHWVRQVPGKGL
EWVSGIDWNSGVIGYADSVKGRFTISRDNVKNSLYLQMTSLRAED
TAVYFCARDILPSNFDGKKIIVFQPPAKRDLDNYYGMDVWGQGTT
VTVSS
SEQ ID NO: 43)
V_{L} chain of F7
QPGLTQPASVSGSPGQSVTISCTGTSSDVGGYNLVSWYQQHPGKA
PKLMIYEGSKRPSGISNRFSGSKSGNTASLTISGLQAEDEADYFC
SSYTITDVVVFGGGTKLTVL
SEQ ID NO: 44)

TABLE 23
S1-RBD-R3-T1-G1 Ab (Ab_35) Variable Region
amino acid sequences

V_{H} chain of G1
EVQLVQSGAEVKKPGESLRISCKASGYSFTSNWIGWVRQMPGKGLEWM
GSIFPGDSDTKYSPSFQGQVTISADRSISTAYLQWSGLKASDTAMYYC
ARESYNAYGSWGQGTLVTVSS
(SEQ ID NO: 47)
V_{L} chain of G1
QPGLTQPPSASGTPGQRVTISCSGSSSNIGSNPVNWYQQLPGTAPKLLI
YSNNQRPSGVPDRFSGSKSGTSASLAISGLRSEDEADYYCAAWDDSLSG
VVFGGGTKLTVL
(SEQ ID NO: 48)

TABLE 24
S1-RBD-R3-T1-C2 Ab Variable Region amino
acid sequences

V_{H} chain of RBD-R3-T1-C2
EVQLVQSGAEVKKPGASVKVSCKASGYTFTSYGISWVRQAPGQGLEWM
GWISAYNGNTKYAQKLQGRVTMTTDTSTSTAYMELRSLRSDDTAVYYC
ARGFPQLGSDYWGQGTLVTVSS
(SEQ ID NO: 49)
V_{L} chain of RBD-R3-T1-C2
QPVLTQPHSVSESPGKTVTISCTRSSGSIASNYVQWYQQRPGSAPTTV
IYEDNQRPSGVPDRFSGSIDSSSNSASLIISGLMTEDEADYYCQSYDS
TNWVFGGGTKLTVL
(SEQ ID NO: 50)

TABLE 25
S1-RBD-R3-T1-H8 Ab Variable Region amino
acid sequences

V_{H} chain of RBD-R3-T1-H8
EVQLVQSGAEVEKPGSSVKVSCKASGGTFSSYAISWVRQAPGQGLEWM
GWISGYNGNTRYAQKFQGRVTLTIDTSSSTAYMELSSLRSEDTAVYYC
ARQMKDSGNYWEYYYYGMDVWGQGTMVTVSS
SEQ ID NO: 51)
V_{L} chain of RBD-R3-T1-H8
QPGLTQPPSVSVAPGQTATITCGGDNIGSESVHWYQQKAGQAPVLVVY
EDRGRPSGIPERFSGSNSGNTATLTISRVEAGDEADYYCQVWNPSGSL
QYVFGPGTRLSV_{L} (SEQ ID NO: 52)

TABLE 26
S1-RBD-R3-E1-E8 Ab (Ab 37) Variable Region
amino acid sequences

V_{H} chain of RBD-R3-E1-E8
QVQLVQSGAEVKKPGASVKVSCKASGYTFTSYGISWVRQAPGQGLE
WMGWISTYNGNTNYAQKLQGRVTMTTDTSTSTAYMEVRSLRSDDTA
VYYCARDVFGHFDYWGQGTLVTVSS
(SEQ ID NO: 53)
V_{L} chain of RBD-R3-E1-E8
NFMLTQPHSVSESPGKTVLISCTRSSGNIATNYVQWYQQRPGSAPT
TVIYEDNQRPSGVPDRFSGSIDSSSNSASLTISGLKTEDEADYYCK
SYDDGNHVFGGGTKLTVL
(SEQ ID NO: 54)

TABLE 27
RBD-R3-T1-H2 Ab Variable Region amino
acid sequences

V_{H} chain of RBD-R3-T1-H2
QVTLKESGPTLVKPTQTLTLTCTFSGFSLTTTGVSVGWIRQPPGKA
LEWLALIHWDDDKRYSPSLRSRLTITRDTSKNQVVLTVTDMDPADT
GTYYCASFIMTVYAEYFEDWGQGTLVTVSS
(SEQ ID NO: 55)
V_{L} chain of RBD-R3-T1-H2
EIVLTQSPATLSVSPGERATLSCRASQSVSSNLPWYQQKPGQAPRL
LMYDVSTRATGIPPRFSGSGSGTEFSLTISSLQSEDFAVYYCQQRG
VWPLTFDGGTNVEIK
(SEQ ID NO: 56)

TABLE 28
S1-RBD-R3-T1-B7 Ab Variable Region amino
acid sequences

V_{H} chain of RBD-R3-T1-B7
QVTLKESGPALVKSTQTLTLTCTISGFSLSTSAMCVSWIRQSPGKA
LEWLALIDWDNDRYYTTSLKTRLTITKDTSKNQVVLTMTSMDPLDT
ATYYCAHSPYDSIWGSFRPSVYYFDYWGQGTLVTVSS
(SEQ ID NO: 57)
V_{L} chain of RBD-R3-T1-B7
NFMLTQPHSVSESPGKTITISCTRTSGSIVSSYVQWYQQRPGSFPI
TVIYEHNQRPSGVPYRFSGSIDRSSNSAALTISDLKTEDEADYYCQ
SYDSQNGVFGGGTKLTVL
(SEQ ID NO: 58)

TABLE 29
S1-RBD-R3-E1-E5 Ab Variable Region amino
acid sequences

V_{H} chain of RBD-R3-E1-E5
QVQLVQSGGGLVKPGGSLRLSCAASGFTFSDYYMTWFRQAPGKGLE
WISYISSSSSDTKYADSVKGRFTISRDNAKNSLYLQMDSLRAEDTA
VYYCAMPTREPAYWGQGTLVTVSS
(SEQ ID NO: 59)
V_{L} chain of RBD-R3-E1-E5
QSALTQPASVSGSPGQSITISCTGTSSDLGTYNYVSWYQQHPGKAP
KLMIYDVFKRPSGVPDRFSGSKSGNTASLTISGLQAEDEADYYCSS
YTSSSTYVFGTGTKVTVL
(SEQ ID NO: 60)

TABLE 30
S1-R3-T1-H6 Ab (Ab_40) Variable Region
amino acid sequences

V_{H} chain of RBD-R3-T1-H6
QVQLVQSGGGVVQPGKSLRLSCAASGFAFSDFPVHWVRQAPGKGL
EWVAVISYDGSLKYYADSVKGRFTLSRDNSKNTVYLQLSSLRRED
TAVYYCAREGVSNSRPFDHWGHGTLVTVSS
(SEQ ID NO: 61)
V_{L} chain of RBD-R3-T1-H6
SYELTQPPSVSVAPGQTARITCGGDSIGTKSVHWYQQKSGQAPVL
VVYDDDDRPSGIPERFSGSNSGNTATLTISRVEAGDEADYYCQVW
ESDDDDLVFGGGTKLTVL
(SEQ ID NO: 62)

TABLE 31
S1-RBD-R3-E1-C6 Ab Variable Region amino
acid sequences

V_{H} chain of RBD-R3-E1-C6
QVQLVQSGGGVVQPGRSLRLSCAASGFTFSSYAMHWVRQAPGKGLEYV
SAISSNGGSTYYADSVKGRFTISRDNGKNSLYLQMSSLRAEDTAVYYC
TRDLWSGSADSFDIWGQGTMVTVSS
(SEQ ID NO: 63)
V_{L} chain of RBD-R3-E1-C6
SSELTQDPAVSVALGQTVKITCQGDSLRRYYASWYQQKPGQAPVRVI
YGKNNRPSGIPDRFSGSSSGNTASLTITGAQAEDEADYYCNSRDISD
NQWQWIFGGGTKLAVL
SEQ ID NO: 64)

TABLE 32
S1-RBD-R3-E1-F2 Ab Variable Region amino
acid sequences

V_{H} chain of RBD-R3-E1-F2
QVQLVQSGGGLVQPGGSLRLSCAASGFPFNAYYMSWVRQAPGKGLEWVAN
INQDGSEKYYVDSVKGRFTISRDNAKNSLYLQMNGLRAEDTAVYYCARLY
WWGMDVWGQGTTVTVSS
(SEQ ID NO: 65)
V_{L} chain of RBD-R3-E1-F2
QSALTQPPSASGSPGQSLTISCTGTSSDVGGYKYVSWYQHHPDKAPKLLI
YDVNNRPSGVSSRFSGSKSGNTASLTISGLQAEDEADYYCSSYTGRMNLY
VFGTGTEVTPR
SEQ ID NO: 66)

TABLE 33
S1-RBD-R3-T1-C3 Ab Variable Region amino
acid sequences

V_{H} chain of RBD-R3-T1-C3
QVQLVQSGGGLVQPGRSLRLSCAASGFTFDDYAMHWVRQVPGKGLEWVS
GIDWNSGVIGYADSVKGRFTISRDNAKNSLYLQMNSLRAEDTALYYCAK
DAYSYGFLGAFDIWGQGTMVTVSS
(SEQ ID NO: 67)
V_{L} chain of RBD-R3-T1-C3
LPVLTQPPSVSVAPGQTASITCGGDNIRTKGVHWYQQKPGQAPLLVIYYA
SDRPSGIPERFSGSSSGNTATLTISRVEAGDEADYYCQVWDSSSDLVVFG
GGTTLTVL
(SEQ ID NO: 68)

TABLE 34
S1-RBD-R3-T1-G12 Ab Variable Region amino
acid sequences

V_{H} chain of RBD-R3-T1-G12
QVQLVQSGGGLVQPGGSLRLSCATSGFTFDDYAMHWVRQAPGKGLEWVS
GISWNSGSIGYVDSVKGRFTISRDNRNNKVYLQMNNLRAEDTAVYYCAR
DWWGSIDHWGLGTLVTVSS
(SEQ ID NO: 69)
V_{L} chain of RBD-R3-T1-G12
QSALTQPRSVSGSPGQSVTISCTGTSSDVGGYDYVSWYQQHPGKAPKLI
IYDVSKRPSGVPDRFSGSKSGNTASLTVSGLQAEDEADYYCSSYTSSSP
VVFGGGTKLTVL
(SEQ ID NO: 70)

TABLE 35
S1-RBD-R3-E1-F1 Ab Variable Region amino
acid sequences

V_{H} chain of RBD-R3-E1-F1
EVQLVESGPGLVKPSGTLSLTCAVSGGSISSSNWWSWVRQPPGKGLEWI
GEIYHSGSTNYNPSLKSRVTISVDTSKNQFSLKLSSVTAADTAVYYCAR
RGGTYHRGAFDIWGQGTMVTVSS
(SEQ ID NO: 72)
V_{L} chain of RBD-R3-E1-F1
QSVLTQPPSASGSPGQSVTISCTGTSRDVGSYDLVSWYQQHPGKAPKLM
IYEGSRRPSGVSSRFSGSKSGNTASLTISGLQAEDEADYYCSSYTSSNS
LVFGTGTKVTVL
(SEQ ID NO: 71)

TABLE 36
S1-RBD-R3-E1-H8 Ab Variable Region amino
acid sequences

V_{H} chain of RBD-R3-E1-H8
QVTLKESGPGLVNPSETLSLTCTVSGASISNSFWSWIRQSPGKGLEWIGY
TSYSGNSIYNPSLKSRLTMSIDTSKNQLSLNLRSLTAADTAVYYCARREW
IKGHFDYWGQGTLVTVSS
(SEQ ID NO: 73)
V_{L} chain of RBD-R3-E1-H8
NFMLTQPHSVSESPGKTVTISCTGSGGSIASNYVQWYQQRPGSAPTTVIY
EDNQRPSGVPDRFSGSIDSSSNSASLTISGLKTEDEADYYCQSYDSSNPV
VFGGGTKLTVL
(SEQ ID NO: 74)

TABLE 37
S1-RBD-R3-T1-F1 Ab Variable Region amino
acid sequences

V_{H} chain of RBD-R3-T1-F1
EVQLVESGPGLMKPSETLFLSCSVSGGSFTTHSWNWIRQTPGKPLEWMG
IILPGGATNKNPPLLSRVSISSDPSNNEFSLTLTSVTAADTAVYYCARG
PGILSYWSGGTLATVSS
(SEQ ID NO: 75)
V_{L} chain of RBD-R3-T1-F1
QPGLTQTPSPSGTPGQRVTISCSGSSSIGSNDVTWYQQLPGTAPKLLIY
SNNQRPSGVPDRFSASRSGTSPSLAITGLQAEDEADYYCAWDDSLSAVV
FGGGTKMTVL
(SEQ ID NO: 76)

TABLE 38
S1-R3-T1-B10 Ab Variable Region amino acid sequences

V_{H} chain of R3-T1-B10
EVQLVESGPGLVSPSATLFLTCSVSGGSFRTHSWNWIRQAPGKPLEWMGVIHHSGA
TNKNPSLKSRVTISSETSDNKFSLTLTSVTAEDTAVYYC [symbol] WSRGTLGTV
SS
(SEQ ID NO: 77)
V_{L} chain of R3-T1-B10
LPVLTQPPSASGTPGQRVTISCSGSSSNIGSNTVNWYHQLPRTAPKLLIYINNHRPSG
VPDRFSGS*SGTSASLAITVIQSEDEADYYC [symbol] FGSGTKVTVL
(SEQ ID NO: 78)

TABLE 39
RBD-R3-E1-D12 Ab Variable Region amino acid sequences

V_{H} chain of RBD-R3-E1-D12
EVQLVESGPGVVSPSATLFLTCSVSGGSIRTHSWNWIRQPPGKPLEWIGVIHHSGAT
NKNPSLKSRVTISSKTSDNKFSLTLTSVTAEDTAVYYC [symbol] WSRGTLGTVS
S
(SEQ ID NO: 79)
V_{L} chain of RBD-R3-E1-D12
LPVLTQPPSASGTPGQRVTISCSGSSSNIGSNTVNWYHYLPRTAPKLLIYINNHRASR
VPDRFSGS*SGTSASLAITVIQSEDEADYYC [symbol] FGSGTKVTVL
(SEQ ID NO: 80)

TABLE 40
RBD-R3-T1-C5 Ab Variable Region amino acid sequences

V_{H} chain of RBD-R3-T1-C5
EVQLVESGPGVVSPSATLFLTCSVSGGSIRTHSWNWIRQAPGKALEWIGFIHHSGAT
NNNPSLKSRVTISSDTSKNEFSLTLTSVTAADTAVYYC [symbol] WSRGTLV
TVSS
(SEQ ID NO: 81)
V_{L} chain of RBD-R3-T1-C5
LPVLTQPPSASGTPGQRVTISCSGSSSNIGSNTVNWYQQLPGTAPKLLIYSNNQRPSR
VPDRFSGSKTGTSPSLAISVLQSEDEADYYC [symbol] FGSGTKSPSY
(SEQ ID NO: 82)

TABLE 41
S1-RBD-R3-T1-B4 Ab Variable Region amino acid sequences

V_{H} chain of RBD-R3-T1-B4
QVQLVQSGAEVKKPGESLKISCKGSGYSFTSYWIGWVRQMPGKGLEWMGIIYPGD
SDTRYSPSFQGQVTISADKSISTAYLQWSSLKASDTAMYYC [symbol] WGQ
GTMVTVSS
(SEQ ID NO: 83)
V_{L} chain of RBD-R3-T1-B4
QPVLTQPPSASGTPGQRVTISCSGSSSNIGSNPVNWYQQLPGTAPKLLIYNNNQRPS
GVPDRFSGSKSGTSASLAISGLQSEDEADYYC [symbol] FGGGTKLTVL
(SEQ ID NO: 84)

TABLE 42
S1-RBD-R3-E1-E7 Ab Variable Region amino acid sequences

V_{H} chain of RBD-R3-E1-E7
QVQLVQSGAEVKKPGNSLKISCKGSRYSFSNYWIAWVRQMPGKGLEWLGSIYPYD
SDTRYSPSLQGQVTISVDKSLSTAYLQWRSLKASDTAMYYCA [symbol] WGQ
GTTVTVSS
(SEQ ID NO: 85)
V_{L} chain of RBD-R3-E1-E7
SSELTQDPAVSVALGQTVRITCQGDSLRSYYASWYQQKPGQAPVLVIYGKNNRPS
GIPDRFSGSSSGNTASLTITGAQAEDEADYYC [symbol] FGGGTKLTVL
(SEQ ID NO: 86)

TABLE 43
S1-RBD-R3-T1-C8 Ab Variable Region amino acid sequences

V_{H} chain of RBD-R3-T1-C8
EVQLVQSGAEVKKPGESLKISCKGSGYSFTSYWIGWVRQMPGKGLEWMGIIYPGD
SDTRYSPSFQGQVTISADKSISTAYLQWSSLKASDTAMYYC [symbol] WGQ
GTLVTVSS
(SEQ ID NO: 87)
V_{L} chain of RBD-R3-T1-C8
QPVLTQPPSASGTPGQRVTISCSGSSSNIGSNPVNWYQQLPGTAPKLLIYDNNQRYP
GVPDRFSGSKSGTSASLAISGLRSEDEADYYC [symbol] FGGGTKLTVL
(SEQ ID NO: 88)

TABLE 44
S1-RBD-R3-T1-D7 Ab (Ab_34) Variable Region amino acid
sequences

V_{H} chain of RBD-R3-T1-D7
QVQLVQSGVEVKKPGESLKISCKGSGYSFTSYWIGWVRQMPGKGLEWMGIIYPGD
SDTRYSPSFQGQVTISADKSISTAYLQWSSLKASDTAMYYC [symbol] WGQ
GTLVTVSS
(SEQ ID NO: 89)
V_{L} chain of RBD-R3-T1-D7
QPGLTQPPSASGTPGQGVTISCSGSSSNIGGNSVHWYQQLPGTAPKLLIYRNNQRPS
GVPDRFSGSKSGTSASLAISGLRSEDEADYYC [symbol] FGGGTKLTVL
(SEQ ID NO: 90)

TABLE 45
RBD-R3-E1-F5 Ab (Ab_36) Variable Region amino acid
sequences

V_{H} chain of RBD-R3-E1-F5
QVQLQQSGPGLVKPSQTLSLTCAIFGDSVTSNSAAWNWIRQSPSRGLEWLGRTYYS
SKWYNDYAVSVKSRVTINADTSKNQLSLQLNSVTPEDTAVYYC [symbol]
WGQGTLVTVSS
(SEQ ID NO: 91)
V_{L} chain of RBD-R3-E1-F5
NFMLTQPHSVSESPGKTVTISCTGSSGSIASNYVQWLQQRLGSAPTTVIYEDNQRPS
GVPDRFSGSIDSSSNSASLTISGLKTEDEADYYC [symbol] FGGGTKLTVL
(SEQ ID NO: 92)

TABLE 46
Ab_13 Variable Region amino acid sequences

VH chain of Ab_13
QVTLKESGPKLVKPTQTLTLTCTFSGFSLSTSGVGVGWIRQPPGKALEWLALIYWD
DDKRYSPSLKSRLTIAKDTSKYQVVLTMTNMDPVDTATYYC [symbol] W
GQGTMVTVSA
(SEQ ID NO: 722)
VL chain of Ab_13
NFMLTQPHSVSESPGKTVTISCTRSSGSIASNYVQWYQQRPGSAPTTVIYEDNQRPS
GVPDRFSGSIDSSSNSASLTISGLKTEDEADYYC [symbol] FGGGTKLTVL
(SEQ ID NO: 723)

TABLE 47
Ab_14 Variable Region amino acid sequences

V_{H} chain of Ab_14
QVQLQQSGPGLVKPSQTLSLTCAISGDSVSSNSAAWNWIRQSPARGLEWLGRTYY
RSRWYNDYAISMKSRITINPDTSKNQFSLQLESVTPEDTAVYYC [symbol] WGQ
GTLVTVSS
(SEQ ID NO: 724)
V_{L} chain of Ab_14
QSVLTQPPSASGSPGQSVTISCTGTSSDVGAYNFVSWYQHHPGKAPKLIIYDFNKRP
SGVPDRFSGSKSGNTASLTVSGLQADDEADYYC [symbol] FGGGTKLTVL
(SEQ ID NO: 725)

TABLE 48
Ab_15 Variable Region amino acid sequences

V_{H} chain of Ab_15
QVQLVQSGGGLVKPGRSLRLSCTASGFTFGDYAMSWFRQAPGKGLEWVGFIRSKA
YGGTTGYAASVKGRFTISRDDSKSIAYLQMNSLKTEDTAVYYC [symbol] WG
KGTTVTVSS
(SEQ ID NO: 726)
V_{L} chain of Ab_15
NFMLTQPHSVSESPGKTVTISCTRSSGTIASNYVQWYQQRPGSAPTTVIYEDNQRPS
GVPDRFSGSIDRSSNSASLTISGLTPDDEADYYC [symbol] FGTGTKVTVL
(SEQ ID NO: 727)

TABLE 49
Ab_16 Variable Region amino acid sequences

V_{H} chain of Ab_16
QVQLVQSGGGVVQPGRSLRLSCAASGFTFSNYGMHWVRQAPGKGLEWVALIWER
GSKKDYADSVKGRFTVSRDNSKNTLYLQMNSLRPEDTAVYFC [symbol] [symbol]
WGQGTLVTVSS
(SEQ ID NO: 728)
V_{L} chain of Ab_16
QSVLTQPPSVSGSPGQRVTMSCTGSSSNIGAGYDVHWYQQVPGAAPRLLIYGTNNR
PSGVPDRFSGSKSGTSASLTITGLQAEDEADYYC [symbol] FGTGTKVTVL
(SEQ ID NO: 729)

TABLE 50
Ab_17 Variable Region amino acid sequences

V_{H} chain of Ab_17
QVQLVQSGGGVVQPGRSLRLSCAASGFTFDDYAMHWVRQVPGKGLEWVSGIDW
NSGVIGYADSVKGRFTISRDNVKNSLYQMNSLRTEDSALYYC [symbol] [symbol]
WGQGTTVTVSS
(SEQ ID NO: 730)
V_{L} chain of Ab_17
QSALTQPASVSGSPGQSITISCTGTSSDVGGSKYVSWYQQHPGKAPKVMIYDVTKR
PSGVPDRFSGSKSGNTASLTISGLQAEDEADYYC [symbol] FGGGTKLTVL
(SEQ ID NO: 731)

TABLE 51
Ab_18 Variable Region amino acid sequences

V_{H} chain of Ab_18
EVQLVQSGGGVVQPGRSLRVSCAASGFSFSRYGMHWVRQAPGKGLEWVAFIRHD
GSKKYYADSVEGRFTISRDNSRNTVSLEMNSLRGEDTAVYYC [symbol] W
GQGTLVTVSS
(SEQ ID NO: 732)
V_{L} chain of Ab_18
NFMLTQPHSMSGSAGKTVTVSCIRSSGSIANNFVQWYQQRPGSAPTTVIYEDNQRP
SGVPDRFSGSIDSSSNSASLTISGLKTEDEADYYC [symbol] FGGGTRLTVL
(SEQ ID NO: 733)

TABLE 52
Ab_19 Variable Region amino acid sequences

V_{H} chain of Ab_19
EVQLVESGAEVKKPGESLKISCKGSGYSFTSYWIGWVRQMPGKGLEWMGIIYPGD
SDTRYSPSFQGQVTISADKSISTAYLQWSSLKASDTAMYYC [symbol] WGQG
TMVTVSS
(SEQ ID NO: 734)
V_{L} chain of Ab_19
QPGLTQPPAASGTPGQRVTVSCSGASANIGSNAVSWFKQFPETAPRLLISGNTHRPS
GVPDRVSGSKSGTSASLTISGLQSDDEADYYC [symbol] FGSGTKVTVL
(SEQ ID NO: 735)

TABLE 53
Ab 20 Variable Region amino acid sequences

V_{H} chain of Ab 20
QVQLVQSGGGSVKPGGSLRLSCAASGYRLSDYYMHWVRQA
PGKGLEWVANIKQDGSEKYYVDSVKGRFTISRDNAKNSLY
LQMNSLRAEDTAVYYCARVRGWSRGYFDYWGQGTLVTVSS
(SEQ ID NO: 736)
V_{L} chain of Ab 20
NFMLTQPHSVSESPGKTVTISCTGSSGSIASNYVQWYQQR
PGSAPTTVIYEDNQRPSGVPDRFSGSIDSSSNSASLLISG
LKTEDEADYYCQSYDSSNHWVFGGGTKLTVL
(SEQ ID NO: 737)

TABLE 54
Ab 21 Variable Region amino acid sequences

V_{H} chain of Ab 21
QVQLVQSGGGLVQPGGSLRLSCATSGFTFDDYAMHWVRQA
PGKGLEWVSGISWNSGSIGYVDSVKGRFTISRDNRNNKVY
LQMNNLRAEDTAVYYCARDWWGSIDHWGLGTLVTVSS
(SEQ ID NO: 738)
V_{L} chain of Ab 21
QSALTQPRSVSGSPGQSVTISCTGTSSDVGGYDYVSWYQQ
HPGKAPKLIIYDVSKRPSGVPDRFSGSKSGNTASLTVSGL
QAEDEADYYCSSYTSSSPVVFGGGTKLTVL
(SEQ ID NO: 739)

TABLE 55
Ab 22 Variable Region amino acid sequences

V_{H} chain of Ab 22
QVQLVQSGGGVVQPGRSLRLSCAASGFTFSHYDMHWVRQA
PGKGLEWLAVIGYDGTNLYYADSVKGRFTISRDKSKNTLY
LQINSLRAEDTAVYYCARAANYYDSSGYGRADAFDIWGQG
TTVTVSS
(SEQ ID NO: 740)
V_{L} chain of Ab 22
NFMLTQPHSVSESPGKTVIISCTRTTGSIAGNYVQWYRQR
PGSAPTTVIYDDNQRPAGVPDRFSGSVDSSSNSASLTITG
LKTEDEADYYCQSYDSGNRGVFGTGTKLTVL
(SEQ ID NO: 741)

TABLE 56
Ab 23 Variable Region amino acid sequences

V_{H} chain of Ab 23
EVQLVQSGAEVKKPGSSVKVSCRSSGGTFSTYGITWVRQA
PGQGLEWMGRIIPSLGIPNYAQKFQGRVTITADTSVSTAW
MELSSLESDDTAIYYCARENIDLATNDFWGQGTLVTVSS
(SEQ ID NO: 742)
V_{L} chain of Ab 23
QSALTQPPSASGSPGQSVTISCTGTSRDIGAYGYVSWYQQ
VPGKAPKLIIYEVRNRPSGVSSRFSGSKSGNTASLTISGL
QAEDEADYYCSSYTSSSTLDVVFGGGTKLTVL
(SEQ ID NO: 743)

TABLE 57
Ab 24 Variable Region amino acid sequences

V_{H} chain of Ab 24
QVQLVQSGAEVKTPGSSVKVSCKASGGTFSSSGVSWVRQA
PGQGLEWMGGIIPMLGTPNYAQKFQGRITITADEATSTVY
MALSSLRSEDTAMYYCARDGGNYDYWGQGTLVTVSS
(SEQ ID NO: 744)
V_{L} chain of Ab 24
QPVLTQPPSASGTPGQRVSISCSGSSSNIGRNAVDWYHQV
PGTAPQLLIYSNNERSSGVPDRFSASRSGNTASLTIIGLQ
PEDEADYYCSAWDTSLSTWVFGGGTKLTVL
SEQ ID NO: 745)

TABLE 58
Ab 25 Variable Region amino acid sequences

V_{H} chain of Ab 25
QVQLVQSGGGLVQPGGSLRLSCAASGFTFSSYWMSWVRQA
PGKGLEWVANIKQDGSEKYYVDSVKGRFTISRDNAKNSLY
LQMNSLRAEDTAVYYCARGFYYYGAFDIWGQGTTVTVSS
(SEQ ID NO: 746)
V_{L} chain of Ab 25
NFMLTQPHSVSESPGKTVTISCTGSSGSIASNYVQWYQQR
PGSAPTTVIYEDNQRPSGVPDRFSGSIGSSSNSASLTISG
LKTEDEADYYCQSYDSSNHWVFGGGTKLTVL
SEQ ID NO: 747)

TABLE 59
Ab 26 Variable Region amino acid sequences

V_{H} chain of Ab 26
QVQLVQSGGGLVQPGRSLRLSCAASGFTFDDYAMHWVRQV
PGKGLEWVSGIDWNSGVIGYADSVKGRFTISRDNAKNSLY
LQMNSLRAEDTALYYCAKDAYSYGFLGAFDIWGQGTMVTV
SS
(SEQ ID NO: 748)
V_{L} chain of Ab 26
LPVLTQPPSVSVAPGQTASITCGGDNIRTKGVHWYQQKPG
QAPLLVIYYASDRPSGIPERFSGSSSGNTATLTISRVEAG
DEADYYCQVWDSSSDLVVFGGGTTLTVL
(SEQ ID NO: 749)

TABLE 60
Ab 27 Variable Region amino acid sequences

V_{H} chain of Ab 27
QVQLVQSGTEVKKPGASVKVSCKASGYSFTGSHLHWVRQA
PGQGLEWMGWINPDSGVINYAQKFQGRVTLTRDTSISTAY
MELSGLRSDDTAVYYCARDKAIGYVWALDYWGQGTLVTVS
S
(SEQ ID NO: 750)
V_{L} chain of Ab 27
QSALTQPPSVSGSPGQSVTISCTGTSSDVGTYNRVSWYQQ
PPGKAPKLMIYEVSNRPSGVSNRFSGSKSGNTASLTISGL
QAEDEADYYCSSYTRTFTYVFGTGTKVTVL
(SEQ ID NO: 751)

TABLE 61
Ab 28 Variable Region amino acid sequences

V_{H} chain of Ab 28
QVTLKESGPGLVNPSETLSLTCTVSGASISNSFWSWIRQS
PGKGLEWIGYTSYSGNSIYNPSLKSRLTMSIDTSKNQLSL
NLRSLTAADTAVYYCARREWIKGHFDYWGQGTLVTVSS
(SEQ ID NO: 752)
V_{L} chain of Ab 28
NFMLTQPHSVSESPGKTVTISCTGSGGSIASNYVQWYQQR
PGSAPTTVIYEDNQRPSGVPDRFSGSIDSSSNSASLTISG
LKTEDEADYYCQSYDSSNPVVFGGGTKLTVL
SEQ ID NO: 753)

TABLE 62
Ab 38 Variable Region amino acid sequences

V_{H} chain of Ab 38
QVTLKESGPTLVKPTQTLTLTCTFSGFSLTTSGVSVGWIR
QPPGKALEWLALIHWDDDKRYSPSLRSRLTITRDTSKNQV
VLTVTDMDPADTGTYYCASFIMTVYAEYFEDWGQGTLVTV
SS
SEQ ID NO: 981)
V_{L} chain of Ab 38
EIVLTQSPATLSVSPGERATLSCRASQSVSSNLAWYQQKP
GQAPRLLMYDVSTRATGIPARFSGSGSGTEFSLTISSLQS
EDFAVYYCQQRGAWPLTFGGGTKVEIK
(SEQ ID NO: 982)

TABLE 65
wcS2-T4-E7 Variable Region amino acid sequences

V_{H} chain of T4-E7
EVQLVQSGGGVVQPGRSLRLSCAASGFTFSSYAMHWVRQA
PGKGLEWVAVISYDGSNKYYADSVKGRFTISRDNSKNTLY
LQMNSLRAEDTAVYYCARVNSGSYYGAFDIWGQGTTVTVS
S
SEQ ID NO: 997)
V_{L} chain of T4-E7
NFMLTQPHSVSESPGKTVTMSCTRSSGDIATRHVQWYQQR
PGSAPTTVIYESNQRPSGVSGRFSGSIDSSSNSASLTISG
LQPEDEADYYCQSYDSTNPWVFGGGTKLTVL
(SEQ ID NO: 998)

TABLE 66
wcS2-E1-A9 Variable Region amino acid sequences

V_{H} chain of El-A9
QVQLV*SGGGLVQPGRSLRLSCAASGFTFDDYAMHWVRQA
PGKGLEWVSGISWNSGSIGYADSVKGRFTISRDNAKNSLY
LQMDSLRAEDTAVYYCARDGGIQLSSFEYWGQGTLVTVSS
SEQ ID NO: 999)
V_{L} chain of El-A9
QPVLTQPPSASGSPGQSVTISCTGTSSDVGAYNYVSWYQQ
HPDKAPKLLIYEVSKRPSGVPDRFSGSKSGNTASLTVSGL
QADDEADYYCSSYAGTRKYYVFGGGTKVTVL
SEQ ID NO: 1000)

TABLE 67
wcS2-T4-H8 Variable Region amino acid sequences

V_{H} chain of T4-H8
QV*LVQSGGGVVQPGRSLRLSCAASGFTFSSYGMHWVRQA
PGKGLEWVAVISYDGSNKYYADSVKGRFTISRDNSKNTLY
LQMNSLRAEDTAVYYCARGSGSYLTDFDYWGQGTLVTVSS
(SEQ ID NO: 1001)
V_{L} chain of T4-H8
ETTLTQSPATLSLSPGERATLSCRASQIVTNNNLAWYQQK
PGQAPRLLIYGASSRATGIPDRFSGSGSGTDFTLTISRLE
PEDFAVYYCQQYYYWPLSFGGGTKVEIK
(SEQ ID NO: 1002)

TABLE 68
wcS2-T3-F5 Variable Region amino acid sequences

V_{H} chain of T3-F5
QVQLVQSGGGVVQPGRSLRLSCAASGFTFSSYAMHWVRQA
PGKGLEWVAVMSYDGSNKYYADSVKGRFTISRDNAKNSLY
LQMNSLRDEDTAVYYCARTGGYLRPIDYWGQGTLVTVSS
SEQ ID NO: 1003)
V_{L} chain of T3-F5
QPVLTQPRSVSGSPGQSVTISCTGTSSDVGGYKYVSWYQHH
PGKAPKLMIYDVSERPSGVSSRFSGSKSGNTASLTISGLQG
EDEADYFCSSFSQTNSYVFGTGTRVAVL
(SEQ ID NO: 1004)

TABLE 69
wcS2-E2-C1 Variable Region amino acid sequences

V_{H} chain of E2-C1
EVQLVQSGGGVVQPGRSLRLSCAASGFTFSSYAMHWVRQA
PGKGLEWVAVISYDGSNKYYADSVKGRFTISRDNSKNTLY
LQMNSLRAEDTAVYYCARDRSGSYWGAFDIWGQGTLVTVS
S
(SEQ ID NO: 1005)
V_{L} chain of E2-C1
QSVLTQPPSASGSPGQSVTISCTGTSSDVGRYKYVSWYQQ
HPGKAPKPMIYEVNKRPSGVPDRFSGSKSGNTASLTVSGL
QAEDEADYYCSSYAGSNNPYVFGTGTKVTVL
(SEQ ID NO: 1006)

TABLE 70
wCS2-T4-C9 Variable Region amino acid sequences

V_{H} chain of T4-C9
QVQLVQSGGGVVQPGRSLRLSCAASGFTFSSYGMHWVRQA
PGKGLEWVAVISYDGSNKYYADSVKGRFTISRDNSKNTLY
LQMNSLRAEDTAVYYCAKGRGSYSTYFDYWGQGTLVTVSS
(SEQ ID NO: 1007)
V_{L} chain of T4-C9
EIVLTQSPATLSLSPGERATLSCRASQNVPSNSLAWYQQK
PGQAPRLLINGASSRANGIPDRFSGSGSGTDFTLTITRLE
PEDFAVYFCQLYDRSSQLAFGGGTKLEIK
SEQ ID NO: 1008)

TABLE 71
wcS2-T3-B9 Variable Region amino acid sequences

V_{H} chain of T3-B9
QVQLVQSGGGVVQPGRSLRLSCAASGFTFSGYAMHWVRQA
PGKGLEWVAVVSYDGSNKYYADSVKGRFTISRDNSKNTLY
LQMNSLRAEDTAVYYCARSEGYSSGWPLDYWGQGTLVTVS
S
(SEQ ID NO: 1009)
V_{L} chain of T3-B9
SSELTQDPAVSVALGQTVRITCQGDSLGTYYASWYQQKPG
QAPLLVIYGKNNRPSGIPDRFSGSSSGNTASLTITGAQAE
DEADYYCNSRNNSGYHEFGGGTKLTVL
(SEQ ID NO: 1010)

TABLE 72
wcS2-E3-C8 Variable Region amino acid sequences

V_{H} chain of E3-C8
QV*LVQSGAEVKKPGESLKISCKGSGYNFIDYWIAWVRQL
PGQGLEWMGIIYPGDSDARYSPSFQGQVTISADKSINTAY
LQWSRLKASDTAKYYCARGYAMDVWGQGTTVTVSS
(SEQ ID NO: 1011)
V_{L} chain of E3-C8
QSVLTQPLSASGTPGHRVTISCSGSQSNIGSNTVNWYQQV
PGTAPKLLIYVNNRRPSGVPDRFSGSKSGTSAALAISGLQ
SEDEADYYCSSYAGSNDYVFGTGTKVTVL
(SEQ ID NO: 1012)

TABLE 73
wCS2-T4-F8 Variable Region amino acid sequences

V_{H} chain of T4-F8
QVELVQSGAEVKEPGASVKVSCKDSGHTFLGHYMHWVRQA
PGQGLEWMGWINPNSGVTKYAEKFQGWVTMTRDTSISTAY
MELSRLKSDDTALYYCARERTTGGAFDIWGQGTMVTVSS
(SEQ ID NO: 1013)
V_{L} chain of T4-F8
NFMLTQPHSVSESPGKTVTISCTRSSGSIASNYVQWYQQR
PGSAPTAVIFGDNQRPSGVPDRFSGSIDSSSNSASLTISG
LRTEDEADYYCQSFDGSYHWVFGGGTKLTVL
(SEQ ID NO: 1014)

TABLE 74
wcS2-T3-F1 Variable Region amino acid sequences

V_{H} chain of T3-F1
EVQLVQSGGGVVQPGRSLRLSCAASGFTFSSYAMHWVRQA
PGKGLEWVAVISYDGSNKYYADSVKGRFTISRDNSKNTLY
LQMNSLRAEDTAVYYCARDRSGSYWGAFDIWGQGTLVTVS
S
SEQ ID NO: 1015)
V_{L} chain of T3-F1
LPVLTQPPSASGTPGQRVTISCSGSSSNIGSNTVNWYQQL
PGTAPKLLIYSNNQRPSGVPDRFSGSKSGTSASLAISGLR
SEDEADYYCATWDDGLSGRVFGGGTNLAVL
(SEQ ID NO: 1016)

TABLE 75
wcS2-E2-B1 Variable Region amino acid sequences

V_{H} chain of E2-B1
QVQLVQSGGGVVQPGRSLRLSCAASGFTFSSYAMHWVRQA
PGKGLEWVAVISYDGSNKYYADSVKGRFTISRDNSKNTLY
LQMNSLRAEDTAVYYCARGDYYYYMDVWGKGTLVTVSS
(SEQ ID NO: 1017)
V_{L} chain of E2-B1
SSELTQDPAVSVALGQTVRITCQGDSLRSYYASWYQQKPG
QAPVLVIYGKNNRPSGIPDRFSGSSSGNTASLTITGAQAE
DEADYYCNSRDSSGNHLRVFGTGTKVTVL
(SEQ ID NO: 1018)

TABLE 76
wcS2-T2-G3 Variable Region amino acid sequences

V_{H} chain of T2-G3
QVQLQESGGGVVQPGRSLRLSCAASGFTFSSYAMHWVRQA
PGKGLEWVAVISYDGSNKYYADSVKGRFTISRDNSKNTLY
LQMNSLRAEDTAVYYCARGLGGGYYYGMDVWGQGTLVTVS
S
(SEQ ID NO: 1019)
V_{L} chain of T2-G3
SYELTQPPSVSQGLRQTATLTCTGNSNNVGNQGAAWLQQH
QGHPPKLLSYRNNIRPSGISERLSASTSGNTASLTITGLQ
PEDEADYYCSAWDNSLGAWVFGEGTKLTVL
(SEQ ID NO: 1020)

TABLE 77
wCS2-T1-A6 Variable Region amino acid sequences

V_{H} chain of T1-A6
QV*LVQSGAEVKKPGESLKISCKGSGYNFIDYWIAWVRQL
PGQGLEWMGIIYPGDSDARYSPSFQGQVTISADKSINTAY
LQWSRLKASDTAKYYCARG*AMDVWGQGTTVTVSS
(SEQ ID NO: 1021)
V_{L} chain of T1-A6
QSVLTQPLSASGTPGHRVTISCSGSQSNIGSNTVNWYQQV
PGTAPKLLIYVNNRRPSGVPDRFSGSKSGTSAALAISGLQ
SEDEADYYCSSYAGSNDYVFGTGTKVTVL
(SEQ ID NO: 1022)

TABLE 78
wcS2-T4-D4 Variable Region amino acid sequences

V_{H} chain of T4-D4
EV*LVQSGGGVVQPGRSLRLSCAASGFTFSSYAMHWVRQA
PGKGLEWVAVISYDGSNKYYADSVKGRFTISRDNSKNTLY
LQMNSLRAEDTAVYYCARGNRGSYYGAFDSWGQGTTATAS
S
(SEQ ID NO: 1023)
V_{L} chain of T4-D4
NFMLTHPHSVS*SPGKTATMSCTRSSGDIATRHVQWYQKR
PGSAPTTVIYESNQRPAGVSGRFSGSIDSSSNSASLTISA
VHPEDEADYYCLTYDITNPWVFGGGTNLTVL
SEQ ID NO: 1024)

TABLE 79
wcS2-T2-D10 Variable Region amino acid sequences

V_{H} chain of T2-D10
EVQLVQSGGGVVQPGRSLRLSCAASGFTFSSYAMHWVRQA
PGKGLEWVAVISYDGSNKYYADSVKGRFTISRDNSKNTLY
LQMNSLRAEDTAVYYCARDNSGSYYGAFDIWGQGTLVTVS
S
(SEQ ID NO: 1025)
V_{L} chain of T2-D10
HFVLTQPPSASESPGKTVTMSCTGTSGDIATKHDDCCHQR
PPGAPPTAMNDNDHKPSAGSADLFAAFDSASTSALIAFSV
LHADDDDDYCCSYDDSTNPCVFGAGTKVTVL
(SEQ ID NO: 1026)

TABLE 80
wcS2-T1-G9 Variable Region amino acid sequences

V_{H} chain of T1-G9
QVQLVQSGGGVVQPGRSLRLSCAASGFTFSSYGMHWVRQA
PGKGLEWVAVISYDGSNKYYADSVKGRFTISRDNSKNTLY
LQMNSLRAEDTAVYYCAKGRGSYSTYFDYWGQGTLVTVSS
(SEQ ID NO: 1027)
V_{L} chain of T1-G9
ETTLTQSPGTLSLSPGERATLSCRASQSISSSYLAWYQQK
PGQAPRLLIYDASNRAPGIPARFSGSGSGTDFTLTISSLE
PEDFAVYYCQQRGNWPLTFGPGTKVHIK
(SEQ ID NO: 1028)

TABLE 81
wcS2-E3-H7 Variable Region amino acid sequences

V_{H} chain of E3-H7
QVQLVQSGGGVVQPGRSLRLSCAASGFTFSIYGMHWVRQA
PGKGLEWVAGISYDGSNKYYADSVKGRFTISRDNSKNTLY
LQMNSLRAEDTAVYYCAKGRGSYSTYFDYWGQGTLVTVSS
(SEQ ID NO: 1029)
V_{L} chain of E3-H7
EIVLTQSPATLALSPGERATLSCRDSQNVPSNSLAWYQQR
PGQAPRLLINGASSRANGIPYRFSGSGSGTDFTLTITRLE
PEDFAVYFCQLYDRSSQLASAERTKMEIK
(SEQ ID NO: 1030)

TABLE 82
wcS2-T2-C11 Variable Region amino acid sequences

V_{H} chain of T2-C11
QVTLKESGPTLVKPTQTLTLTCTFSGFSLSTSGVGVGWIR
QPPGKALEWLALIYWDDDKRYSPSLKSRLTISKDTSRNQV
VLTMTNMDPADTGTYYCAHRRPDWDAFDVWGQGTMVTVSS
(SEQ ID NO: 1031)
V_{L} chain of T2-C11
SSELTQDPAVSVALGQTVRITCQGDSLTIFFANWYQQKPG
QAPILVMSKDTERPSGIPERFSGSSSGTRVTLTISGVQAE
DEADYYCQSADTSGTLKVFGGGTKLTVL
SEQ ID NO: 1032)

TABLE 83
SARS2-R3-G2-P1-H9_PeIB-F_2020-06-19_D08
Variable Region amino acid sequences

V_{H} chain of G2-P1-H9
QVQLVQSGGGLVKPGGSLRLSCSASGFTFSSYAMHWVRQA
PGKGLEWVAVISYDGSNKYYADSVQGRITISRDNSKNTLY
LQMNSLRAEDTAVYFCARSDGYPYEPFDYWGQGTLVTVSS
(SEQ ID NO: 1033)
V_{L} chain of G2-P1-H9
QSALTQPASVSGSPGQSITISCTGTSSDLGGHNFVSWYQQ
HPGKAPKLMIYGVNKRPSGVPDRFSGSKSGNTASLTVSGL
QAEDEADYYCSSYEATHIYVFGTGTKVAVL
(SEQ ID NO: 1034)

TABLE 84
SARS2-R3-G3-P1-F8 PeIB-F 2020-06-19_
F11 Variable Region amino acid sequences

V_{H} chain of G3-P1-F8
QVQLVQSGGGVVQPGRSLRLSCAASGFTFSSYGMHWVRQA
PGKGLEWVAVISYDGSNKYYADSVKGRFTISRDNSKNTLY
LQMNSLRAEDTAVYYCARGSGITGAFRDWGQGTLVTVSS
(SEQ ID NO: 1035)
V_{L} chain of G3-P1-F8
QSALTQPASVSGSPGQSITISCTGTSSDVGSYNLVSWYQQ
HPGKAPKLMISEVSNRPSGVSNRFSGSKSGNTASLTISGL
QAEDEADYYCSSFSSGSIPYVFGAGTKVTVL
(SEQ ID NO: 1036)

TABLE 85
SARS2-R3-G2-P1-B4_PelB-F_2020-06-19_B02
Variable Region amino acid sequences

V_{H} chain of G2-P1-B4
QVQLVQSGGGVVQPGRSLRLSCAASGFTFSSYGMHWVRQAPGKGLEW
VAVISYDGSNKYYADSVKGRFTISRDNSKNTLYLQMNSLRTEDTAVY
YC [symbol] [symbol] WGQGTLVTVSS
(SEQ ID NO: 1037)
V_{L} chain of G2-P1-B4
EIVLTQSPATLSVSPGERATLSCRASQSVSTNLAWYQQKPGQAPRLL
IYGASTRATGIPARFSGSGSGTEFTLTVSRLEPEDFAVYYC [symbol]
FGQGTRVEIR
(SEQ ID NO: 1038)

TABLE 86
SARS2-R3-G3-P1-B2_PelB-F_2020-06-19_A08
Variable Region amino acid sequences

V_{H} chain of G3-P1-B2
QVQLVQSGGGVVQPGRSLRLSCAASGFTFSSYAMHWVRQAPGKGLEW
VAVISYDGSNKYYADSVKGRFTISRDNSKNTLYLQMNSLRAEDTAVY
YC [symbol] [symbol] WGQGTTVTVSS
(SEQ ID NO: 1039)
V_{L} chain of G3-P1-B2
QSVLTQPPSASGTPGQRVTISCSGSSSNIGSNTVNWYQQLPGTAPKL
LIYSNNQRPSGVPDRFSGSKSGTSVSLAISGLQSEDEADYYC [symbol]
FGGGTNLAVL
(SEQ ID NO: 1040)

TABLE 87
SARS2-R3-G3-P1-B3_PelB-F_2020-06-19_B08
Variable Region amino acid sequences

V_{H} chain of G3-P1-B3
QVQLQESGGGVVQPGRALRLSCRASGFNFGTFGMHWVRQAPGKGLEW
VAVISYDGSNKYYADSVKGRFTISRDNSKNTLYLQMNSLRAEDTAVY
YC [symbol] [symbol] WGQGTMVTVSS
(SEQ ID NO: 1041)
V_{L} chain of G3-P1-B3
SYELTQPPSVSVAPGKTANMTCGGNNIGSKSVHWYQQKPGQAPVLVV
YDDTDRPPGIPERFSGSNSGNTATLTISRVEVGDEADYYC [symbol]
FGTGTKVTVL
(SEQ ID NO: 1042)

TABLE 88
SARS2-R3-G1-P3-E8_PelB-F_2020-06-19_H04
Variable Region amino acid sequences

V_{H} chain of G1-P3-E8
*VQLVQSGAEVKKPGSSVKVSCKASGGTFSSYAISWVRQAPGQGLEW
MGGIIPIFGTANYAQKFQGRVTITADKSTSTAYMELSSLRSEDTAVY
YC [symbol] WGQGTMVTVSS
(SEQ ID NO: 1043)
V_{L} chain of G1-P3-E8
SSELTQDPAVSVALGQTVRITCQGDSLRSYYASWYQQKPGQAPVLVI
YGGNNRPSGIPDRISGSSSGNTASLTITGAQAEDEADYYC [symbol]
FGTGTKVTVL
(SEQ ID NO: 1044)

TABLE 89
SARS2-R3-G1-P2-D7_PelB-F_2020-06-19_H09
Variable Region amino acid sequences

V_{H} chain of G1-P2-D7
EVQLVQSGAEVKKPGASVKVSCKASGYTFTSYGISWVRQAPGQGLEW
MGWISAYNGNTNYAQKLQGRVTMTTNTSTNTAYMELRSLRSDDTAGY
YW [symbol] [symbol] WGQGTTVTVSS
(SEQ ID NO: 1045)
V_{L} chain of G1-P2-D7
QSGLTQPPSVSGAPGQRVTISCTGSSSNIGAGYDVHWYQQLPGTAPK
LIIYGNSNRPSGVPDRFSGSKSGTSASLAITGLQAEDEADYYC [symbol]
FGGGTKLTVL
(SEQ ID NO: 1046)

TABLE 90
SARS2-R3-G1-P1-C6_PelB-F_2020-06-19_H02
Variable Region amino acid sequences

V_{H} chain of G1-P1-C6
QVTLKESGPTLVKPTQTLTLTCTFSGFSLSTSGVGVGWIRQPPGKALE
WLALIYWDDDKRYSPSLKSRLTITKDTSKNQVVLTMTNMDPVDTATYY
C [symbol] WGQGTLVTVSS
(SEQ ID NO: 1047)
V_{L} chain of G1-P1-C6
SYELTQPPSVSVSPGQTARITCSGDALPNRYAYWYQQRPGQAPVLVIY
KDSERPSGIPERFSGSSSGTTVTLTISGVQAEDEADYYC [symbol]
FGGGTKVTVL
(SEQ ID NO: 1048)

TABLE 91
SARS2-R3-T-P2-E12_PelB-F_2020-06-19_D07
Variable Region amino acid sequences

V_{H} chain of T-P2-E12
QVQLVQSGGGLVQPGGSLRLSCSASGFTFSSYAMHWVRQAPGKGLEW
VEVISYDGSNKYYAYSVQGRFTISKNNSKNTLYLQMNSLKAEDTAVY
FC [symbol] RGRGTPGTASS
(SEQ ID NO: 1049)
V_{L} chain of T-P2-E12
HTVLTHPAPAAAYPGQTITISCSATSSDLVGHKFVSWYQQHPGKAPT
LVIYEINNRPSGVPDRFSGSIYGNTDSLTVSAGVEDEDDYYC [symbol]
S*TGTEVAFI
(SEQ ID NO: 1050)

TABLE 92
SARS2-R3-G2-P1-D12_PelB-F_2020-06-19_
H04 Variable Region amino acid sequences

V_{H} chain of G2-P1-D12
QVQLVQSGGGVVQPGRSLRLSCAASGFIFSSYGMHWVRQAPGKGLEW
VAVISYDGSNKYYADSVKGRFTISRDNSKNTLYLQMNSLRVEDTAVY
YC [symbol] [symbol] WGQGTMVTVSS
(SEQ ID NO: 1051)
V_{L} chain of G2-P1-D12
DIVMTQTPSTLSASVGDRVTISCRASENVNNWVAWYQQKPGKVPELL
MYKASRLEPGVPSRFSGSGSGTEFTLTISNLQPEDFATYYC [symbol]
FGQGTKVDIK
(SEQ ID NO: 1052)

TABLE 93
SARS2-R3-G1-P2-E8_PelB-F_2020-06-19_E10
Variable Region amino acid sequences

V_{H} chain of G1-P2-E8
EVQLVQSGGGVVQPGRSLRLSCAASGFTFSSYAMHWVRQAPGKGLEW
VAVISYDGSNKYYADSVKGRFTISRDNSKNTLYLQMNSLRAEDTAVY
YC [symbol] [symbol] WGQGTLVTVSS
(SEQ ID NO: 1053)
V_{L} chain of G1-P2-E8
NFMLTQPHSVSESPGKTVTISCTRSSGSIASNYVQWYQQRPGSSPTA
VIYAHNQRPSEVPDRFSGSIDISSNSASLTISGLKTEDEADYYC [symbol]
FGGGTKLTVL
(SEQ ID NO: 1054)

TABLE 94
SARS2-R3-G1-P1-A10_PelB-F_2020-06-19_H01
Variable Region amino acid sequences

V_{H} chain of G1-P1-A10
EVQLVQSGGGVVQPGRSLTLSCAASGFTFSSYAMHWVRQAPGKGLEW
VAVISYDGSNKYYADSVKGRFTISRDNSKNTLYLQMNSLRAEDTAVY
YC [symbol] [symbol] WGQGTTVTVSS
(SEQ ID NO: 1055)
V_{L} chain of G1-P1-A10
ETTLTQSPGTLSLSPGERATLSCRASQSVRSNLAWYQQKPGQAPRLL
IYGVSTRATGIPARFSGSGSGTDFTLTISRLEPEDFAVYFC [symbol]
FGQGTKLEIK
(SEQ ID NO: 1056)

TABLE 95
SARS2-R3-G1-P3-B5_PelB-F_2020-06-19_D02
Variable Region amino acid sequences

V_{H} chain of G1-P3-B5
QVQLVQSGGGVVQPGRSLRLSCAASGFTFSSYGMHWVRQAPGKGLEW
VALISYDGSNKYYADSVKGRFTISRDNAKNSLYLQMNSLRAEDTAVY
YC [symbol] WGQGTLVTVSS
(SEQ ID NO: 1057)
V_{L} chain of G1-P3-B5
QSALTQPPSASGSPGQSVTISCTGTSSDVGSYNRVSWYQQPPGTAPK
LMIYEVSNRPSGVPDRFSGSKSGNTASLTISGLQAEDEADYYC [symbol]
FGTGTKVTVL
(SEQ ID NO: 1058)

TABLE 96
SARS2-R3-G3-P1-A2_PelB-F_2020-06-19_B07
Variable Region amino acid sequences

V_{H} chain of G3-P1-A2
QVQLVQSGTEVRQPGASVKVSCKASGYTFTSYGISWVRQAPGQGLEW
MGGIIPIFGTANYAQKFQGRVTITADESTSTAYMELSSLRSEDTAVY
YC [symbol] [symbol] DVWGQGTTVTVSS
(SEQ ID NO: 1059)
V_{L} chain of G3-P1-A2
NFMLTQPHSVSGSPGKTVTISCTRDIGDIARNYVQWYQQRPGSSPTT
VIYEDDRRPSGVPDRFSGSVDRSSNSASLTISGLDTEDEADYYC [symbol]
FGGGTKLTVL
(SEQ ID NO: 1060)

TABLE 97
SARS2-R3-G3-P1-D1_PelB-F_2020-06-19_G09
Variable Region amino acid sequences

V_{H} chain of G3-P1-D1
EVQLVQSGGGVVQPGRSLRLSCAASGFTFSSYAMHWVRQAPGKGLEW
VAVISYDGSNKYYADSVKGRFTISRDNSKNTLYLQMNSLRAEDTAVY
YC [symbol] WGKGTLVTVSS
(SEQ ID NO: 1061)
V_{L} chain of G3-P1-D1
QPVLTQPPSVSGAPGQSVTISCIGSSSNIGAGYHVQWYQQVPGTAPK
LLIYGNQNRPSGVPDRFSGSKSGTSASLAISGLRSEDEADYFC [symbol]
FGTGTKVNVL
(SEQ ID NO: 1062)

TABLE 98
SARS2-R3-G1-P2-D4_PelB-F_2020-06-19_G09
Variable Region amino acid sequences

V_{H} chain of G1-P2-D4
QVQLVQSGGGVVRPGRSLRLSCAASGFTFSSYALHWVRQAPGKGLEW
VAVISYDGSHKYYADSVKGRFTISRDNAKNSLYLQMNSLRAEDTAVY
YC [symbol] [symbol] WGQGTMVTVSS
(SEQ ID NO: 1063)
V_{L} chain of G1-P2-D4
QPGLTQPPSVSKDLRQTATLTCTGNSNNVGHEGAAWLQQHQGHPPKL
LSYKNDNRPSGISERFSASTSGNTASLTITGLQPEDEADYYC [symbol]
FGGGSRLTVL
(SEQ ID NO: 1064)

TABLE 99
SARS2-R3-G1-P1-B6_PelB-F_2020-06-19_E02
Variable Region amino acid sequences

V_{H} chain of G1-P1-B6
EVQLVQSGGGVVQPGRSLRLSCAASGFTFSSYAMHWVRQAPGKGLEW
VAVISYDGSNKYYADSVKGRFTISRDNSKNTLYLQMNSLRAEDTAIY
YC [symbol] [symbol] WGQGTLVTVSS
(SEQ ID NO: 1065)
V_{L} chain of G1-P1-B6
QPVLTQPPSASGTPGQRVTISCSGGRSNIGSNTVNWYQQLPGTAPKL
LIYSNNHRPSGVPDRFSGSKSGNTASLTISGLQAEDEADYYC [symbol]
FGGGTKLTVL
(SEQ ID NO: 1066)

TABLE 100
SARS2-R3-G3-P1-G8_PelB-F_2020-06-25_A05 Variable Region
amino acid sequences

V_{H} Chain of G3-P1-G8
QVTLKESGPALVKPTQTLTLTCTFSGFSLSTSGVGVGWIRQPPGKALEWLALIYWD
DDKRYSPSLKSRLTITKDTSKNQVVLTMTNMDPVDTATYYC [symbol] WG
QGTLVTVSS
(SEQ ID NO: 1067)
V_{L} chain of G3-P1-G8
QPVLTQPPSVSVSPGQTASITCSGDNLGEIYASWYQQKPGQSPVLVIYQDKKRPSGI
PERFSGSNSGNTATLTISETQAMDEADYYC [symbol] FGTGTKVTVL
(SEQ ID NO: 1068)

TABLE 101
SARS2-R3-G1-P1-F11_PelB-F_2020-06-25_B05 Variable Region
amino acid sequences

V_{H} chain of G1-P1-F11
QVTLKESGPTLVKPTQTLTLTCTLSGFSLTTSGVGVGWIRQPPGKALEWLALIYWD
DDKRYSPSLKSRLTITKDTSKNQVVLTMTNMDPVDTATYYC [symbol] W
GQGTTVTVSS
(SEQ ID NO: 1069)
V_{L} chain of G1-P1-F11
SYELTQPPSVSVSPGQTATITCSGDALPDKYAYWYRQRPGQAPVLVIYKDSERPSGI
PERFSGSSSGTTVTLTISGVQAEDEADYYC [symbol] FGGGTKLTVL
(SEQ ID NO: 1070)

TABLE 102
SARS2-R3-T1-P3-F4_PelB-F_2020-06-20_C03 Variable Region
amino acid sequences

V_{H} chain of T1-P3-F4
QVQLVQSGGGVVQPGRSRRLSCTASGITFYSYGMHWVRQAPGKGLEWVSTISNIY
NTHYADSVKGRFTISRDNSKNTLYLQMKSLRAEDTATYYC [symbol] WGQGTL
VTVSS
(SEQ ID NO: 1071)
V_{L} chain of T1-P3-F4
QSVLTQPPSASGSPGQSVTISCTGTSSDVGGYNHVSWYQQHPGKAPKVLIYDVSKR
PSGVPDRFSGSKSGNTASLTVSGLQAEDEADYYC [symbol] FGTGTEVTVL
(SEQ ID NO: 1072)

TABLE 103
SARS2-R3-G1-P2-B8_PelB-F_2020-06-25_C06 Variable Region
amino acid sequences

V_{H} chain of G1-P2-B8
QVQLVQSGGGLVQPGGSLRLSCAASGFTFSSYAMSWVRQAPGKGLEWVSAISGSG
GSTYYADSVKGRFTISRDNSKNTLYLQMNSLRAEDTGVYYC [symbol] WGQ
GTLVTVSS
(SEQ ID NO: 1073)
V_{L} chain of G1-P2-B8
QSVLTQPPSASGSPGHSVTISCTGTSGDVGGYNSVSWYQHHPGKAPKLMIYEVTKR
PSGVPDRFSGSKSGNTASLSVSGLQAEDEADYYC [symbol] FGTGTKVTVL
(SEQ ID NO: 1074)

TABLE 104
SARS2-R3-G1-P2-D3_PelB-F_2020-06-25_C12 Variable Region
amino acid sequences

V_{H} chain of G1-P2-D3
QVQLVQSGGGVVQPGRSLRLSCAASGFTFSSYAMHWVRQAPGKGLEWVAVISYD
GSNKYYADSVKGRFTISRDNSKNTLYLQMNSLRAEDTAVYYC [symbol] [symbol]
WGQGTLVTVSS
(SEQ ID NO: 1075)
V_{L} chain of G1-P2-D3
NFMLTQPHSVSESPGKTVAISCTRSSGSIASNYVQWYQQRPGSSPTTVIYEDNQRPS
GVPDRFSGSIDSSSNSASLTISGLKTEDEADYYC [symbol] FGGGTKLTVL
(SEQ ID NO: 1076)

TABLE 105
SARS2-R3-G1-P4-C5_PelB-F_2020-06-25_C07 Variable Region
amino acid sequences

V_{H} chain of G1-P4-C5
EVQLVQSGGGVVQPGRSLRLSCAASGFTFSSYGMHWVRQAPGKGLEWVSYISSSS
KYIYYADSVKGRFTISRDNAKNSLYLQMNSLRAEDTAVYYC [symbol] W
GQGTLVTVSS
(SEQ ID NO: 1077)
V_{L} chain of G1-P4-C5
LPMLTQPPSMSGTPGQRVTISCSGSSSNIGSNTVNWYQQVPGTAPKVLIYSNNQRPS
GVPDRFSGSKSGTSASLAISGLRSEDEADYSC [symbol] IGGGTKLTVL
(SEQ ID NO: 1078)

TABLE 106
SARS2-R3-G1-P2-D6_PelB-F_2020-06-25_D02 Variable Region
amino acid sequences

V_{H} chain of G1-P2-D6
QVQLVQSGAEVKKPGASVKVSCKASGYTFSSYAFSWVRQAPGQGLEWMGWISAF
NGNTDYAQNFQGRVTMTTDTSTNTAYMELRSLRSDDTAVYYC [symbol] WG
QGTLVTVSS
(SEQ ID NO: 1079)
V_{L} chain of G1-P2-D6
SYELTQPPSVSKGLRQTATLTCTGNSNNVGHEGASWLQHHQGHPPKLLSYRNKNR
PSGISERFSASRSGNTASLTITGLQPEDEADYYC [symbol] FGTGTKVTVL
(SEQ ID NO: 1080)

TABLE 107
SARS2-R3-G1-P1-B7_PelB-F 2020-06-25_A03 Variable Region
amino acid sequences

V_{H} chain of G1-P1-B7
EVQLVQSGAEVKKPGASVKVSCKASGYTFTSYYMHWVRQAPGQGLEWMGIINPS
GGSTSYAQKFQGRVTMTRDTSTSTVYMELSSLRSEDTAVYYC [symbol]
DIWGQGTTVTVSS
(SEQ ID NO: 1081)
V_{L} chain of G1-P1-B7
SYELTQPPSVSVSPGQTATITCSGDKLEDKFVSWYQQKPGHSPLLVIYEDAKRPSGIP
ERFSGSNSGNRAILTINGTQALDEADYYC [symbol] FGGGTKLAVL
(SEQ ID NO: 1082)

TABLE 108
SARS2_95_PelB-F_2020-06-25_G11 Variable Region amino
acid sequences

V_{H} chain of G11
QVQLVQSGGGVVQPGRSLRLSCAASGFTFSNYAMHWVRQAPGKGLEWVAVISYD
GSTEYYADSVKGRFTISRDNSKKMLYLQMNSLTAEDTAVYYC [symbol] [symbol]
WGQGTTVTVSS
(SEQ ID NO: 1083)
V_{L} chain of G11
ETTLTQSPGTLSLSPGERATLSCRASQSVSSTYLAWYQQKPGQAPRLLIYGASNRAT
GIPDRFSGSGSGTDFTLTISSLQPEDFATYYC [symbol] FGQGTKLEIN
(SEQ ID NO: 1084)

TABLE 109
SARS2_73_PelB-F_2020-06-25_F09 Variable Region
amino acid sequences

V_{H} chain of F09
QVQLVQSGGGLVQPGGSLRLSCAASGFTFSSYEMNWVRQAPGKGLEWVAVISYD
GSNKYYADSVKGRFTISRDNSKNTLYLQMNSLRAEDTAVYYC [symbol] WG
QGTLVTVSS
(SEQ ID NO: 1085)
V_{L} chain of F09
DIVMTQSPATLSVSPGERATLSCRASQSVRDGYLAWYQQRPGQAPRLLISGASTRA
TDIPDRFTGSGSGTDFTLTISSLEPEDFAVYY [symbol] FGQGTKVESK
(SEQ ID NO: 1086)

TABLE 110
SARS2-R3-G3-P1-G9_PelB-F_2020-06-25_B05 Variable Region
amino acid sequences

V_{H} chain of G3-P1-G9
QVQLVQSGGGLVKPGGSLRLSCAASGFTFSSYAMHWVRQAPGKGLEWVAVISYD
GSNKYYADSVKGRFTISRDNSKNTLYLQMNSLRAEDTAVYYC [symbol]
WGQGTLVTVSS
(SEQ ID NO: 1087)
V_{L} chain of G3-P1-G9
QSVLTQPPSASGSPGQSVTVSCTGTSDDVGGFAHVSWYQQHPGKAPKLLIHDVSKR
PSGVPDRFSASKSGNTASLTISGLQPEDEGDYFC [symbol] FGGGTTLTVL
(SEQ ID NO: 1088)

TABLE 111
SARS2-R3-G1-P3-G7_PelB-F_2020-06-25_H01 Variable Region
amino acid sequences

V_{H} chain of G1-P3-G7
QVQLVQSGAEVKKPGASVKVSCKASGYTFTGYYMHWVRQAPGQGLEWMGRINP
NSGGTNYAQKFQGRVTMTRDTSISTAYMELSRLRSDDTAVYYC [symbol] [symbol]
WGQGTTVTVSS
(SEQ ID NO: 1089)
V_{L} chain of G1-P3-G7
QPVLTQPPSVSKDLRQTATLTCTGNSNNVGDQGTAWLQQHQGHPPKLVSYRNNN
RPSGVSERFSASRSGNTASLTITGLQAEDEADYYC [symbol] FGGGTKLTVL
(SEQ ID NO: 1090)

TABLE 112
SARS2-R3-G1-P4-A2_PelB-F_2020-06-25_A06 Variable Region
amino acid sequences

V_{H} chain of G1-P4-A2
QVQLQQSGAEVKKPGSSVKVSCKASGSTFNNYAVSWVRQAPGQGPEWMGRIIPIV
DIANYAQRFQGRVTITADESTNTAYMELSSLRSEDTAVYYC [symbol] W
GKGTTVTVSS
(SEQ ID NO: 1091)
V_{L} chain of G1-P4-A2
NFMLTQPHSVSESPGKTVTISCTRSSGSIASNYVQWYQQRPGSAPTTVIYEDNQRPS
GVPDRFSGSIDSSSKTASLIISGLETEDEADYYC [symbol] FGGGTKLTVP
(SEQ ID NO: 1092)

TABLE 113
SARS2-R3-G3-P1-G1_PelB-F_2020-06-25_F04 Variable Region
amino acid sequences

V_{H} chain of G3-P1-G1
EVQLVQSGAEVKKPGASVKVSCKASGYTFTSYGISWVRQAPGQGLEWMGWISAY
NGNTNYAQKLQGRVTMTTDTSTSTAYMELRSLRSDDTAVYYC [symbol] [symbol]
WGQGTTVTVSS
(SEQ ID NO: 1093)
V_{L} chain of G3-P1-G1
QSVLTQPPSVSGAPGQRVTISCTGSSSNIGAGYDVHWYQQLPGTAPKLIIYGNSNRP
SGVPDRFSGSKSGTSASLAITGLQAEDEADYYC [symbol] FGGGTKLTVL
(SEQ ID NO: 1094)

TABLE 114
SARS2-R3-G3-P1-F1_PelB-F_2020-06-25_C04 Variable Region
amino acid sequences

V_{H} chain of G3-P1-F1
QVQLVQSGGGVVQPGRSLRLSCAASGFTFSSYAMHWVRQAPGKGLEWVAVISYD
GSNKYYADSVKGRFTISRDNSKNTLYLQMNSLRDEDTAVYYC [symbol] [symbol]
WGQGTLVTVSS
(SEQ ID NO: 1095)
V_{L} chain of G3-P1-F1
QSALTQPASVSGSPGQSITISCTGTSSDLGGHNFVSWYQQHPASAPKLIIYDVYNRPS
GVSNRFSGSESGDTASLTISGLRAEDEADYFC [symbol] FGTGTKVTVL
(SEQ ID NO: 1096)

TABLE 115
SARS2-R3-G1-P3-H10_PelB-F_2020_Jun. 25_H10
Variable Region amino acid sequences

V_{H} chain of G1-P3-H10
QVQLQESGAGLLRPSETLSLTCAVYGGSFSSYHWSWIRQPPGKGLEWIGE
IDHYGSPNYNPSFQSRVAMSRDTPKNQFSLKLSSVTAADTAVYYC [symbol]
WGQGTLVTVSS
(SEQ ID NO: 1097)
V_{L} chain of G1-P3-H10
LPVLTQPPSASETPGQRVTISCSGGRSNIGINSVNWYQQLPGTAPKLLIY
RNNQRPSGVPDRFFGSKSGTSASLAISGLQSEDEADYYC [symbol]
FGGGTKLTVL
(SEQ ID NO: 1098)

TABLE 116
SARS2-85_PelB-F_2020_Jun. 25_G10 Variable Region
amino acid sequences

V_{H} chain of G10
QVQLV*SGAEVKKPGESLKISCQGSGYNFNDYWVGWVRQKPGKGLEWMGI
IYPGDSDTRKNPSFEGQVTMSVDKSLHSVYLHWTSLKVSDTAKYYC [symbol]
WGQGTLGTVSS
(SEQ ID NO: 1099)
V_{L} chain of G10
QPVLTQPPSVSGAPGQSVTISCTGSSSNIGNYGVHWYQQLPGTAPKLLIY
SNNQRPSGVPDRFSGSKSGTSASLAISGLQSEDEADYYC [symbol]
FGGGTKVTVL
(SEQ ID NO: 1100)

TABLE 117
SARS2-R3-G1-P4-A3_PelB-F_2020_Jun. 25_B06 Variable
Region amino acid sequences

V_{H} chain of G1-P4-A3
QVQLVQSGAEVKKPGASVKVSCKASGYTFSNYDINWVRQATGQGLEWMGW
MNPNSGNTGYAQSFQGRVTFTRDTSINTAYMELSSLRSEDTAVYYC [symbol]
WGQGTTVTVSS
(SEQ ID NO: 1101)
V_{L} chain of G1-P4-A3
QSVLTQPPSASGTPGGRVTISCSGSTSNIGRNKVYWYQRLPGTAPKLLIY
LNNQRPSGVPDRFSGSKSGTSASLAISGLQSEDEADYFC [symbol]
FGTGTKVTVL
(SEQ ID NO: 1102)

TABLE 118
SARS2-R3-G1-P3-H6_PelB-F_2020_Jun. 25_H08 Variable
Region amino acid sequences

V_{H} chain of G1-P3-H6
QVQLVQSGAEVKKSGESLEISCKGSGYSFTNYWISWVRQMPGKGLEWMGR
IDPRDSYTNYSPSFQGHVTITVDKSTGAAYLHWSSLKASDTGMYYC [symbol]
WGQGTMVTVSS
(SEQ ID NO: 1103)
V_{L} chain of G1-P3-H6
SSELTQDPAVSVALGQTVRIICQGDSLSRYYANWYQQKSGQAPILVMYGK
DIRPSIPDRFSGSSSGNTASLTITGAQ.EDEADYYC [symbol] VRRRI
KLTVL
(SEQ ID NO: 1104)

TABLE 119
SARS2-R3-G3-P1-G5_PelB-F_2020_Jun. 25_H04 Variable
Region amino acid sequences

V_{H} chain of G3-P1-G5
EVQLVQSGAEVKKPGESLRISCKGSGYTFTNYWIGWVRQMPGKGLEWMGV
IYPGDSDTIYSPSFQGQVTISADKSISTAYLQWSSLKASDTAIYYC [symbol]
WGQGTMVTVSS
(SEQ ID NO: 1105)
V_{L} chain of G3-P1-G5
QSALTQPASVSGSPGQSITISCTGTSSDVGGYNYVSWYQQHPGKAPKLMI
YDVNYRPSGISHRFSGSKSGNTASLTISGLQAEDEADYYC [symbol]
FGTGTKVTVL
(SEQ ID NO: 1106)

TABLE 120
SARS2-R3-G1-P4-C10_PelB-F_2020_Jun. 25_F07
Variable Region amino acid sequences

V_{H} chain of G1-P4-C10
EVQLVESGGGQVKPGGSLRISCAASGFTFENYNMHWVRQAPGKGLEWVSS
ITGHAYYTYYADSLKGRFNISRDNAKKLLYLQLSSLSAEDTALYFC [symbol]
WGHGTLVTVSS
(SEQ ID NO: 1107)
V_{L} chain of G1-P4-C10
SYELTQPPSVSVSPGQTASITCSGDGLPKHYAYWYQQRPGQAPVLLIYKD
TERSSGIPERFSGSGSGTTVTLTINGVQAEDEADYYC [symbol] FG
GGTKLTVL
(SEQ ID NO: 1108)

TABLE 121
SARS2-R3-G1-P1-F10_PelB-F_2020_Jun. 25_B04
Variable Region amino acid sequences

V_{H} chain of G1-P1-F10
QVQLVQSGAEVKKPGSSVKVSCKASGYTFTSYGISWVRQAPGQGLELMGW
ISAYNGNTNYAQKLQGRVTMTTDTSTSTAYMELRSLRSGDTAVYYC [symbol]
WGQGTLVTVSS
(SEQ ID NO: 1109)
V_{L} chain of G1-P1-F10
LPVLTQPPSVSGAPGQRVAISCTGSSSNIGRGYNVHWYQQLPGAAPKLLIY
GNSNRPSGVPDRFSGSKSGTSASLAISGLRSEDEADYYC [symbol]
FGGGTRLTVL
(SEQ ID NO: 1110)

TABLE 122
SARS2-R3-T1-P4-H4_PelB-F_2020_Jun. 20_H04_Variable
Region amino acid sequences

V_{H} chain of T1-P4-H4
EVQLVESGPGLVKPSQTLSLTCTVSGGSISSGGYYWSWIRQHPGKGLEWI
GYIYYSGSTYYNPSLKSRVTISVDTSKNQFSLKLSSVTAADTAVYYC [symbol]
WGQGTMVTVSS
SEQ ID NO: 1111)
V_{L} chain of T1-P4-H4
QSVLTQPPSVSVAPGQTARITCGGDNIGHKGVHWYQQKAGQAPVLVVHDD
SDRPSGIPERFSGSNSGNTATLTISRVEAGDEADYYC [symbol]
FGGGTKLTVL
(SEQ ID NO: 1112)

TABLE 123
SARS2-R3-G2-P1-E8_PelB-F_2020_Jun. 25_A02 Variable
Region amino acid sequences

V_{H} chain of G2-P1-E8
QVQLVQSGGGVVRPGRSLRLSCAASGFTFSSYAMHWVRQAPGKGLEWVAV
ISYDGSNKYYADSVKGRFTISRDNAKNSLYLQMNSLRAEDTAVYYC
Q [symbol] WGGTLVTVSS
(SEQ ID NO: 2785)
V_{L} chain of G2-P1-E8
DIVMTQSPLSLPVTPGEPASISCRSSQSLLHSNGYNYLDWYLQKPGQSPQ
LLIHFGSNRASGVPDRFSGSGSDTDFTLKISRVEAEDVGVYYC [symbol]
FGQGTRLDIK
(SEQ ID NO: 1344)

TABLE 124
SARS2-R3-G1-P1-C3_PelB-F_2020_Jun. 25_A07 Variable
Region amino acid sequences

V_{H} chain of G1-P1-C3
QVQLVQSGGGLVQPGGSLRLSCAASGFTFSSYGMHWVRQAPGKGLEWVAV
ISYDGSNKYYADSVKGRFTISRDNSKNTLYLQMNSLRAEDTAVYYC [symbol]
WGQGTLVTVSS
SEQ ID NO: 1113)
V_{L} chain of G1-P1-C3
QSVLTQPPSVSGAPGERVTFSCTGTTSNIGAGYDVHWYQQLPGTAPKLLI
YDNNIRPSGVPDRFSASKSGTSASLAITGLQSEDEGDYYC [symbol]
FGGGTKLTVL
(SEQ ID NO: 1114)

TABLE 125
SARS2-R3-G3-P1-G2_PelB-F_2020_Jun. 25_G04 Variable
Region amino acid sequences

V_{H} chain of G3-P1-G2
EVQLVQSGGGLVQPGGSLRLPCSASGFDFSNYDMHWVRQAPGKGLEHISI
ITRDGGRTDYAESVKGRFTISRDNSKNTLYLQMTSLREEDTAVYYC [symbol]
WGQGTLVTVSS
(SEQ ID NO: 1115)
V_{L} chain of G3-P1-G2
QSVLTQPPSASGSPGQSVTISCTGTSSDVGGYNHVSWYQQHPGKAPKVLI
YDVSKRPSGVPDRFSGSKSGNTASLTVSGLQAEDEADYYC [symbol]
FGTGTEVTVL
(SEQ ID NO: 1116)

TABLE 126
SARS2-R3-G1-P4-D1_PelB-F_2020_Jun. 25_A08 Variable
Region amino acid sequences

V_{H} chain of G1-P4-D1
QVQLVQSGAEVKKPGSSAKVSCKASGGTFNNYAISWVRQAPGQGPEWMGR
IIPIVDIANYAQRFQGRVTITADESTNTAYMELSSLRSEDTAVYYC [symbol]
WGKGTLVTVSS
(SEQ ID NO: 1117)
V_{L} chain of G1-P4-D1
NFMLTQPHSVSESPGKTVTISCTRSSGSIASNYVQWYQQRPGSAPTTVIY
EDDQRPSGVPDRFSASIDSSSNSASLTISGLKAEDEADYYC [symbol]
FGGGTKLTVL
(SEQ ID NO: 1118)

The amino acid sequences of the heavy and light chain complementary determining regions of the anti-SARS CoV antibodies are shown in Table 63A-B below:

TABLE 63A
Heavy chain (V_{H}) complementary determining regions (CDRs) of the COVID-
19 antibodies.

| Sequence ID | CDR1-IMGT | CDR2-IMGT | CDR3-IMGT |
|---|---|---|---|
| S1-R3-T1- | GGSIRTHS | IHHSGAT | ARGPGILSY |
| H7 (Ab_12) | (SEQ ID NO: 93) | (SEQ ID NO: 94) | (SEQ ID NO: 95) |
| RBD-R3-E1- | GGSISSYY | IYTSGST | ARDVGFGWFDR |
| G7 (Ab2-10) | (SEQ ID NO: 96) | (SEQ ID NO: 97) | (SEQ ID NO: 98) |
| S1-RBD-R3- | GGSIRTHS | IHHSGAT | ARGPGILSY |
| E1-D8 | (SEQ ID NO: 99) | (SEQ ID NO: 100) | (SEQ ID NO: 101) |
| (Ab_3) | | | |
| S1-RBD-R3- | GDSVSSYSDA | TYYRSKWYN | AREIVATTPFRNYYYGMDV |
| T1-C7 | (SEQ ID NO: 102) | (SEQ ID NO: 103) | (SEQ ID NO: 104) |
| RBD-R3-T1- | GFTFSHYD | IGYDGTNL | ARAANYYDSSGYGRADAF |
| F4 (Ab2-2) | (SEQ ID NO: 105) | (SEQ ID NO: 106) | DI |
| | | | (SEQ ID NO: 107) |
| RBD-R3-E1- | GFTFSDFP | ISYDGNIK | ARGGSSFDI |
| A5 | (SEQ ID NO: 108) | (SEQ ID NO: 109) | (SEQ ID NO: 110) |
| S1-RBD-R3- | GFSLSTTGVG | IYWNDDK | ARISGSGYFYPFDI |
| T1-F5 (Ab2- | (SEQ ID NO: 111) | (SEQ ID NO: 112) | (SEQ ID NO: 113) |
| 7) | | | |
| S1-R3-T1- | GFTFTTYG | ISYDGSIK | ARVGDSSSYYGIDA |
| A12 (Ab_5) | (SEQ ID NO: 114) | (SEQ ID NO: 115) | SEQ ID NO: 116) |
| S1-R3-T1- | GFTFSSHA | ISYDGSYT | ARDWVNFGMDV |
| A6 (Ab_4) | (SEQ ID NO: 117) | (SEQ ID NO: 118) | (SEQ ID NO: 119) |
| S1-RBD-R3- | GYTFSDYY | IDPNSGGT | ARDRGRGGQAGAFDY |
| T1-A5 | (SEQ ID NO: 120) | (SEQ ID NO: 121) | (SEQ ID NO: 978) |
| S1-RBD-R3- | GFTFSSYA | ISYGGSNK | AKVRGSGWYWGSAFDI |
| T1-B3 | (SEQ ID NO: 122) | (SEQ ID NO: 123) | (SEQ ID NO: 124) |
| (Ab_1) | | | |
| S1-RBD-R3- | GYSFTGSH | INPDSGVI | ARDKAIGYVWALDY |
| T1-E7 | (SEQ ID NO: 125) | (SEQ ID NO: 126) | (SEQ ID NO: 127) |
| S1-RBD-R3- | GVSLDTIGMR | IDWDDDK | ARSGLLYDLDV |
| T1-F9 | (SEQ ID NO: 128) | (SEQ ID NO: 129) | (SEQ ID NO: 130) |
| S1-R3-T1- | GFTFSDYP | TSYDGRIK | ARDPGWLRSVGMDV |
| C2 (Ab_6) | (SEQ ID NO: 131) | (SEQ ID NO: 132) | (SEQ ID NO: 133) |
| RBD-R3-E1- | GYSFTSYW | IYPGDSDT | ARGWQWHDY |
| B3 | (SEQ ID NO: 134) | (SEQ ID NO: 135) | (SEQ ID NO: 136) |
| RBD-R3-T1- | GDSVSSRSSA | TYYRSNWNY | VRNMRPDFDL |
| B5 | (SEQ ID NO: 137) | (SEQ ID NO: 138) | (SEQ ID NO: 139) |
| RBD-R3-T1- | GYTFTTSG | ISAYNGNT | ARDFHLYYGMDV |
| H3 (Ab_2) | (SEQ ID NO: 140) | (SEQ ID NO: 141) | (SEQ ID NO: 142) |
| S1-R3-T1- | GGTFSSYA | INPNSGGT | ARGSGGYYLG |
| C4 (Ab_8) | (SEQ ID NO: 143) | (SEQ ID NO: 144) | (SEQ ID NO: 145) |
| S1-RBD-R3- | GGTFSSYT | IIPILGTP | AVGSGWYSGFDY |
| T1-B12 | (SEQ ID NO: 146) | (SEQ ID NO: 147) | (SEQ ID NO: 148) |
| S1-RBD-R3- | GFTFSSYW | IKQDGSEK | ARGFYYYGAFDI |
| T1-G5 | (SEQ ID NO: 149) | (SEQ ID NO: 150) | (SEQ ID NO: 151) |
| S1-RBD-R3- | GFTFDDYA | IDWNSGVI | AKDAYSYGFLGAFDI |
| T1-E2 (Ab7) | (SEQ ID NO: 152) | (SEQ ID NO: 153) | (SEQ ID NO: 154) |
| RBD-R3-T1- | GFTFDDYA | IDWNSGVI | ARDILPSNFDGKKIIVFQPPA |
| F7 | (SEQ ID NO: 155) | (SEQ ID NO: 156) | KRDLDNYYGMDV |
| | | | (SEQ ID NO: 157) |
| S1-RBD-R3- | GYSFTSNW | IFPGDSDT | ARESYNAYGS |
| T1-G1 | (SEQ ID NO: 158) | (SEQ ID NO: 159) | (SEQ ID NO: 160) |
| S1-RBD-R3- | GYTFTSYG | ISAYNGNT | ARGFPQLGSDY |
| T1-C2 | (SEQ ID NO: 161) | (SEQ ID NO: 162) | (SEQ ID NO: 163) |
| S1-RBD-R3- | GGTFSSYA | ISGYNGNT | ARQMKDSGNYWEYYYYG |
| T1-H8 | (SEQ ID NO: 164) | (SEQ ID NO: 165) | MDV |
| | | | (SEQ ID NO: 166) |
| S1-RBD-R3- | GYTFTSYG | ISTYNGNT | ARDVFGHFDY |
| E1-E8 | (SEQ ID NO: 167) | (SEQ ID NO: 168) | (SEQ ID NO: 169) |
| RBD-R3-T1- | GFSLTTTGVS | IHWDDDK | ASFIMTVYAEYFED |
| H2 | (SEQ ID NO: 170) | (SEQ ID NO: 171) | (SEQ ID NO: 172) |
| S1-RBD-R3- | GFSLSTSAMC | IDWDNDR | AHSPYDSIWGSFRPSVYYF |
| T1-B7 | (SEQ ID NO: 173) | (SEQ ID NO: 174) | DY |
| | | | (SEQ ID NO: 175) |
| S1-RBD-R3- | GFTFSDYY | ISSSSSDT | AMPTREPAY |
| E1-E5 | (SEQ ID NO: 176) | (SEQ ID NO: 177) | (SEQ ID NO: 178) |
| S1-R3-T1- | GFAFSDFP | ISYDGSLK | AREGVSNSRPFDH |
| H6 | (SEQ ID NO: 179) | (SEQ ID NO: 180) | (SEQ ID NO: 181) |
| S1-RBD-R3- | GFTFSSYA | ISSNGGST | TRDLWSGSADSFDI |
| E1-C6 | (SEQ ID NO: 182) | (SEQ ID NO: 183) | (SEQ ID NO: 184) |
| S1-RBD-R3- | GFPFNAYY | INQDGSEK | ARLYWWGMDV |
| E1-F2 | (SEQ ID NO: 185) | (SEQ ID NO: 186) | (SEQ ID NO: 187) |
| S1-RBD-R3- | GFTFDDYA | IDWNSGVI | AKDAYSYGFLGAFDI |
| T1-C3 | (SEQ ID NO: 188) | (SEQ ID NO: 189) | (SEQ ID NO: 190) |
| S1-RBD-R3- | GFTFDDYA | ISWNSGSI | ARDWWGSIDH |
| T1-G12 | (SEQ ID NO: 191) | (SEQ ID NO: 192) | (SEQ ID NO: 193) |
| S1-RBD-R3- | GGSISSSNW | IYHSGST | ARRGGTYHRGAFDI |
| E1-F1 | (SEQ ID NO: 194) | (SEQ ID NO: 195) | (SEQ ID NO: 196) |
| S1-RBD-R3- | GASISNSF | TSYSGNS | ARREWIKGHFDY |
| E1-H8 | (SEQ ID NO: 197) | (SEQ ID NO: 198) | (SEQ ID NO: 199) |
| S1-RBD-R3- | GGSFTTHS | ILPGGAT | ARGPGILSY |
| T1-F1 | (SEQ ID NO: 200) | (SEQ ID NO: 201) | (SEQ ID NO: 202) |
| S1-R3-T1- | GGSFRTHS | IHHSGAT | ARGPGILSY |
| B10 | (SEQ ID NO: 203) | (SEQ ID NO: 204) | (SEQ ID NO: 205) |
| RBD-R3-E1- | GGSIRTHS | IHHSGAT | ARGPGILSY |
| D12 | (SEQ ID NO: 206) | (SEQ ID NO: 207) | (SEQ ID NO: 208) |
| RBD-R3-T1- | GGSIRTHS | IHHSGAT | GRGPGILSY |
| C5 | (SEQ ID NO: 209) | (SEQ ID NO: 210) | (SEQ ID NO: 211) |
| S1-RBD-R3- | GYSFTSYW | IYPGDSDT | ARQGDGGGYDY |
| T1-B4 | (SEQ ID NO: 212) | (SEQ ID NO: 213) | (SEQ ID NO: 214) |
| S1-RBD-R3- | RYSFSNYW | IYPYDSDT | ARQGSSQSFDI |
| E1-E7 | (SEQ ID NO: 215) | (SEQ ID NO: 216) | (SEQ ID NO: 217) |
| S1-RBD-R3- | GYSFTSYW | IYPGDSDT | ARRRGSAAAFDT |
| T1-C8 | (SEQ ID NO: 218) | (SEQ ID NO: 219) | (SEQ ID NO: 220) |
| S1-RBD-R3- | GYSFTSYW | IYPGDSDT | ARTTYSYGSFDY |
| T1-D7 | (SEQ ID NO: 221) | (SEQ ID NO: 222) | (SEQ ID NO: 223) |
| RBD-R3-E1- | GDSVTSNSAA | TYYSSKWYN | ARGWLRLSFDP |
| F5 | (SEQ ID NO: 224) | (SEQ ID NO: 225) | (SEQ ID NO: 226) |
| Ab_13 | GFSLSTSGVG | IYWDDDK | ARISGSGYFYPFDI |
| | (SEQ ID NO: 754) | (SEQ ID NO:755) | (SEQ ID NO: 756) |
| Ab_14 | GDSVSSNSAA | TYYRSRWYN | AREIRGFDY |
| | (SEQ ID NO: 757) | (SEQ ID NO: 758) | (SEQ ID NO: 759) |
| Ab_15 | GFTFGDYA | IRSKAYGGTT | TTADDDMDV |
| | (SEQ ID NO: 760) | (SEQ ID NO: 761) | (SEQ ID NO: 762) |
| Ab_16 | GFTFSNYG | IWERGSKK | AREGISMTGAEYFQH |
| | (SEQ ID NO: 763) | (SEQ ID NO: 764) | (SEQ ID NO: 765) |
| Ab_17 | GFTFDDYA | IDWNSGVI | AKDIGPGGSGSYYAFDI |
| | (SEQ ID NO: 766) | (SEQ ID NO: 767) | (SEQ ID NO: 768) |
| Ab_18 | GFSFSRYG | IRHDGSKK | AKDGRLEAALDD |
| | (SEQ ID NO: 769) | (SEQ ID NO: 770) | (SEQ ID NO: 771) |
| Ab_19 | GYSFTSYW | IYPGDSDT | ARRGDLDAFDI |
| | (SEQ ID NO: 772) | (SEQ ID NO: 773) | (SEQ ID NO: 774) |
| Ab_20 | GYRLSDYY | IKQDGSEK | ARVRGWSRGYFDY |
| | (SEQ ID NO: 775) | (SEQ ID NO: 776) | (SEQ ID NO: 777) |
| Ab_21 | GFTFDDYA | ISWNSGSI | ARDWWGSIDH |
| | (SEQ ID NO: 778) | (SEQ ID NO: 779) | (SEQ ID NO: 780) |
| Ab_22 | GFTFSHYD | IGYDGTNL | ARAANYYDSSGYGRADAF |
| | (SEQ ID NO: 781) | (SEQ ID NO: 782) | DI (SEQ ID NO: 783) |
| Ab_23 | GGTFSTYG | IIPSLGIP | ARENIDLATNDF |
| | (SEQ ID NO: 784) | (SEQ ID NO: 785) | (SEQ ID NO: 786) |
| Ab_24 | GGTFSSSG | IIPMLGTP | ARDGGNYDY |
| | (SEQ ID NO: 787) | (SEQ ID NO: 788) | (SEQ ID NO: 789) |
| Ab_25 | GFTFSSYW | IKQDGSEK | ARGFYYYGAFDI |
| | (SEQ ID NO: 790) | (SEQ ID NO: 791) | (SEQ ID NO: 792) |
| Ab_26 | GFTFDDYA | IDWNSGVI | AKDAYSYGFLGAFDI |
| | (SEQ ID NO: 793) | (SEQ ID NO: 794) | (SEQ ID NO: 795) |
| Ab_27 | GYSFTGSH | INPDSGVI | ARDKAIGYVWALDY |
| | (SEQ ID NO: 796) | (SEQ ID NO: 797) | (SEQ ID NO: 798) |
| Ab_28 | GASISNSF | TSYSGNS | ARREWIKGHFDY |
| | (SEQ ID NO: 799) | (SEQ ID NO: 800) | (SEQ ID NO: 801) |
| Ab_38 | GFSLTTSGVS | IHWDDDK | ASFIMTVY AEYFED |
| | (SEQ ID NO: 983) | (SEQ ID NO: 984) | (SEQ ID NO: 985) |
| wcS2-T4-E7 | GFTFSSYA | ISYDGSNK | ARVNSGSYYGAFDI |
| | (SEQ ID NO: 122) | (SEQ ID NO: | (SEQ ID NO: 1163) |
| | | 1143) | |
| wcS2-E1-A9 | GFTFDDYA | ISWNSGSI | ARDGGIQLSSFEY |
| | (SEQ ID NO: 152) | (SEQ ID NO: 192) | (SEQ ID NO: 1164) |
| wcS2-T4-H8 | GFTFSSYG | ISYDGSNK | ARGSGSYLTDFDY |
| | (SEQ ID NO: | (SEQ ID NO: | (SEQ ID NO: 1165) |
| | 1119) | 1143) | |
| wcS2-T3-F5 | GFTFSSYA | MSYDGSNK | ARTGGYLRPIDY |
| | (SEQ ID NO: 122) | (SEQ ID NO: | (SEQ ID NO: 1166) |
| | | 1144) | |
| wcS2-E2-C1 | GFTFSSYA | ISYDGSNK | ARDRSGSYWGAFDI |
| | (SEQ ID NO: 122) | (SEQ ID NO: | (SEQ ID NO: 1167) |
| | | 1143) | |
| wcS2-T4-C9 | GFTFSSYG | ISYDGSNK | AKGRGSYSTYFDY |
| | (SEQ ID NO: | (SEQ ID NO: | (SEQ ID NO: 1168) |
| | 1119) | 1143) | |
| wcS2-T3-B9 | GFTFSGYA | VSYDGSNK | ARSEGYSSGWPLDY |
| | (SEQ ID NO: | (SEQ ID NO: | (SEQ ID NO: 1169) |
| | 1120) | 1145) | |
| wcS2-E3-C8 | GYNFIDYW | IYPGDSDA | ARGYAMDV |
| | (SEQ ID NO: | (SEQ ID NO: | (SEQ ID NO: 1170) |
| | 1121) | 1146) | |
| wcS2-T4-F8 | GHTFLGHY | INPNSGVT | ARERTTGGAFDI |
| | (SEQ ID NO: | (SEQ ID NO: | (SEQ ID NO: 1171) |
| | 1122) | 1147) | |
| wcS2-T3-F1 | GFTFSSYA | ISYDGSNK | ARDRSGSYWGAFDI |
| | (SEQ ID NO: 122) | (SEQ ID NO: | (SEQ ID NO: 1167) |
| | | 1143) | |
| wcS2-E2-B1 | GFTFSSYA | ISYDGSNK | ARGDYYYYMDV |
| | (SEQ ID NO: 122) | (SEQ ID NO: | (SEQ ID NO: 1172) |
| | | 1143) | |
| wcS2-T2-G3 | GFTFSSYA | ISYDGSNK | ARGLGGGYYYGMDV |
| | (SEQ ID NO: 122) | (SEQ ID NO: | (SEQ ID NO: 1173) |
| | | 1143) | |
| wcS2-T1-A6 | GYNFIDYW | TYPGDSDA | ARG*AMDV |
| | (SEQ ID NO: | (SEQ ID NO: | (SEQ ID NO: 1174) |
| | 1121) | 1146) | |
| wcS2-T4-D4 | GFTFSSYA | ISYDGSNK | ARGNRGSYYGAFDS |
| | (SEQ ID NO: 122) | (SEQ ID NO: | (SEQ ID NO: 1175) |
| | | 1143) | |
| wcS2-T2- | GFTFSSYA | ISYDGSNK | ARDNSGSYYGAFDI |
| D10 | (SEQ ID NO: 122) | (SEQ ID NO: | (SEQ ID NO: 1176) |
| | | 1143) | |
| wcS2-T1-G9 | GFTFSSYG | ISYDGSNK | AKGRGSYSTYFDY |
| | (SEQ ID NO: | (SEQ ID NO: | (SEQ ID NO: 1168) |
| | 1119) | 1143) | |
| wcS2-E3-H7 | GFTFSIYG | ISYDGSNK | AKGRGSYSTYFDY |
| | (SEQ ID NO: | (SEQ ID NO: | (SEQ ID NO: 1168) |
| | 1123) | 1143) | |
| wcS2-T2- | GFSLSTSGVG | IYWDDDK | AHRRPDWDAFDV |
| C11 | (SEQ ID NO: 754) | (SEQ ID NO: 755) | (SEQ ID NO: 1177) |
| SARS2-R3- | GFTFSSYA | ISYDGSNK | ARSDGYPYEPFDY |
| G2-P1- | (SEQ ID NO: 122) | (SEQ ID NO: | (SEQ ID NO: 1178) |
| H9_PelB- | | 1143) | |
| F_2020_Jun. | | | |
| 19_D08 | | | |
| SARS2-R3- | GFTFSSYG | ISYDGSNK | ARGSGITGAFRD |
| G3-P1- | (SEQ ID NO: | (SEQ ID NO: | (SEQ ID NO: 1179) |
| F8_PelB- | 1119) | 1143) | |
| F_2020_Jun. | | | |
| 19_F11 | | | |
| SARS2-R3- | GFTFSSYG | ISYDGSNK | ARPQGGAYNGYFDS |
| G2-P1- | (SEQ ID NO: | (SEQ ID NO: | (SEQ ID NO: 1180) |
| B4_PelB- | 1119) | 1143) | |
| F_2020_Jun. | | | |
| 19_B02 | | | |
| SARS2-R3- | GFTFSSYA (SEQ | ISYDGSNK (SEQ | ARSDSGNYYGAFDI (SEQ |
| G3-P1- | ID NO: 122) | ID NO: 1143) | ID NO: 1181) |
| B2_PelB- | | | |
| F_2020_Jun. | | | |
| 19_A08 | | | |
| SARS2-R3- | GFNFGTFG (SEQ | ISYDGSNK (SEQ | ARVRGSGYPHAFDI (SEQ |
| G3-P1- | ID NO: 1124) | ID NO: 1143) | ID NO: 1182) |
| B3_PelB- | | | |
| F_2020_Jun. | | | |
| 19_B08 | | | |
| SARS2-R3- | GGTFSSYA (SEQ | IIPIFGTA (SEQ | ARYKWEPNVGGAFDI (SEQ |
| G1-P3- | ID NO: 143) | ID NO: 1148) | ID NO: 1183) |
| E8_PelB- | | | |
| F_2020_Jun. | | | |
| 19_H04 | | | |
| SARS2-R3- | GYTFTSYG (SEQ | ISAYNGNT (SEQ | AKGGSYGRYGMDV (SEQ |
| G1-P2- | ID NO: 161) | ID NO: 141) | ID NO: 1184) |
| D7_PelB- | | | |
| F_2020_Jun. | | | |
| 19_H09 | | | |
| SARS2-R3- | GFSLSTSGVG | IYWDDDK (SEQ | AKMGNAWTFEH (SEQ ID |
| G1-P1- | (SEQ ID NO: 754) | ID NO: 755) | NO: 1185) |
| C6_PelB- | | | |
| F_2020_Jun. | | | |
| 19_H02 | | | |
| SARS2-R3- | GFTFSSYA (SEQ | ISYDGSNK (SEQ | ARANGCPY*PFYY (SEQ ID |
| T-P2- | ID NO: 122) | ID NO: 1143) | NO: 1186) |
| E12_PelB- | | | |
| F_2020_Jun. | | | |
| 19_D07 | | | |
| SARS2-R3- | GFIFSSYG (SEQ | ISYDGSNK (SEQ | ARCSGGSCYFNGMDV |
| G2-P1- | ID NO: 1125) | ID NO: 1143) | (SEQ ID NO: 1187) |
| D12_PelB- | | | |
| F_2020_Jun. | | | |
| 19_H04 | | | |
| SARS2-R3- | GFTFSSYA (SEQ | ISYDGSNK (SEQ | ARDRSGSYWGAFDI (SEQ |
| G1-P2- | ID NO: 122) | ID NO: 1143) | ID NO: 1167) |
| E8_PelB- | | | |
| F_2020_Jun. | | | |
| 19_E10 | | | |
| SARS2-R3- | GFTFSSYA (SEQ | ISYDGSNK (SEQ | ARDRSGSYWGAFDI (SEQ |
| G1-P1- | ID NO: 122) | ID NO: 1143) | ID NO: 1167) |
| A10_PelB- | | | |
| F_2020_Jun. | | | |
| 19_H01 | | | |
| SARS2-R3- | GFTFSSYG (SEQ | ISYDGSNK (SEQ | ARDYGLRYFDY (SEQ ID |
| G1-P3- | ID NO: 1119) | ID NO: 1143) | NO: 1188) |
| B5_PelB- | | | |
| F_2020_Jun. | | | |
| 19_D02 | | | |
| SARS2-R3- | GYTFTSYG (SEQ | IIPIFGTA (SEQ | ARSMVQGVIRFYGMDV |
| G3-P1- | ID NO: 161) | ID NO: 1148) | (SEQ ID NO: 1189) |
| A2_PelB- | | | |
| F_2020_Jun. | | | |
| 19_B07 | | | |
| SARS2-R3- | GFTFSSYA (SEQ | ISYDGSNK (SEQ | ARSSNYYYMDV (SEQ ID |
| G3-P1- | ID NO: 122) | ID NO: 1143) | NO: 1190) |
| D1_PelB- | | | |
| F_2020_Jun. | | | |
| 19_G09 | | | |
| SARS2-R3- | GFTFSSYA (SEQ | ISYDGSHK (SEQ | ARVLGGSYRMGAFDI (SEQ |
| G1-P2- | ID NO: 122) | ID NO: 1149) | ID NO: 1191) |
| D4_PelB- | | | |
| F_2020_Jun. | | | |
| 19_G09 | | | |
| SARS2-R3- | GFTFSSYA (SEQ | ISYDGSNK (SEQ | SRDAVAGYRGGFDY (SEQ |
| G1-P1- | ID NO: 122) | ID NO: 1143) | ID NO: 1192) |
| B6_PelB- | | | |
| F_2020_Jun. | | | |
| 19_E02 | | | |
| SARS2-R3- | GFSLSTSGVG | IYWDDDK (SEQ | AHLDTFYGMDV (SEQ ID |
| G3-P1- | (SEQ ID NO: 754) | ID NO: 755) | NO: 1193) |
| G8_PelB- | | | |
| F_2020_Jun. | | | |
| 25_A05 | | | |
| SARS2-R3- | GFSLTTSGVG | IYWDDDK (SEQ | AHSRGLWGAFDI (SEQ ID |
| G1-P1- | (SEQ ID NO: | ID NO: 755) | NO: 1194) |
| F11_PelB- | 1126) | | |
| F_2020_Jun. | | | |
| 25_B05 | | | |
| SARS2-R3- | GITFYSYG (SEQ | ISNIYNT (SEQ ID | AKGIAAADY (SEQ ID NO: |
| T1-P3- | ID NO: 1127) | NO: 1150) | 1195) |
| F4_PelB- | | | |
| F_2020_Jun. | | | |
| 20_C03 | | | |
| SARS2-R3- | GFTFSSYA (SEQ | ISGSGGST (SEQ | ANDIQGDPVR (SEQ ID NO: |
| G1-P2- | ID NO: 122) | ID NO: 1151) | 1196) |
| B8_PelB- | | | |
| F_2020_Jun. | | | |
| 25_C06 | | | |
| SARS2-R3- | GFTFSSYA (SEQ | ISYDGSNK (SEQ | ARARGYSYGHHFDY (SEQ |
| G1-P2- | ID NO: 122) | ID NO: 1143) | ID NO: 1197) |
| D3_PelB- | | | |
| F_2020_Jun. | | | |
| 25_C12 | | | |
| SARS2-R3- | GFTFSSYG (SEQ | ISSSSKYI (SEQ | ARDADTSSSESDY (SEQ ID |
| G1-P4- | ID NO: 1119) | ID NO: 1152) | NO: 1198) |
| C5_PelB- | | | |
| F_2020_Jun. | | | |
| 25_C07 | | | |
| SARS2-R3- | GYTFSSYA (SEQ | ISAFNGNT (SEQ | ARDGGKLDY (SEQ ID NO: |
| G1-P2- | ID NO: 1128) | ID NO: 1153) | 1199) |
| D6_PelB- | | | |
| F_2020_Jun. | | | |
| 25_D02 | | | |
| SARS2-R3- | GYTFTSYY (SEQ | INPSGGST (SEQ | ARDLPLRYRPNAFDI (SEQ |
| G1-P1- | ID NO: 1129) | ID NO: 1154) | ID NO: 1200) |
| B7_PelB- | | | |
| F_2020_Jun. | | | |
| 25_A03 | | | |
| 95_PelB- | GFTFSNYA (SEQ | ISYDGSTE (SEQ | ARDPRYCSGAGCYYYGMD |
| F_2020_Jun. | ID NO: 1130) | ID NO: 1155) | V (SEQ ID NO: 1201) |
| 25_G11 | | | |
| 73_PelB- | GFTFSSYE (SEQ | ISYDGSNK (SEQ | ARDPSYGLDY (SEQ ID NO: |
| F_2020_Jun. | ID NO: 1131) | ID NO: 1143) | 1202) |
| 25_F09 | | | |
| SARS2-R3- | GFTFSSYA (SEQ | ISYDGSNK (SEQ | ARDRGGYSGYLDY (SEQ |
| G3-P1- | ID NO: 122) | ID NO: 1143) | ID NO: 1203) |
| G9_PelB- | | | |
| F_2020_Jun. | | | |
| 25_B05 | | | |
| SARS2-R3- | GYTFTGYY (SEQ | INPNSGGT (SEQ | ARESGLLWFGDYYYGMDV |
| G1-P3- | ID NO: 1132) | ID NO: 144) | (SEQ ID NO: 1204) |
| G7_PelB- | | | |
| F_2020_Jun. | | | |
| 25_H01 | | | |
| SARS2-R3- | GSTFNNYA (SEQ | IIPIVDIA (SEQ ID | ARGGSQGAYYMDV (SEQ |
| G1-P4- | ID NO: 1133) | NO: 1156) | ID NO: 1205) |
| A2_PelB- | | | |
| F_2020_Jun. | | | |
| 25_A06 | | | |
| SARS2-R3- | GYTFTSYG (SEQ | ISAYNGNT (SEQ | ARGGSYGRYGMDV (SEQ |
| G3-P1- | ID NO: 161) | ID NO: 141) | ID NO: 1206) |
| G1_PelB- | | | |
| F_2020_Jun. | | | |
| 25_F04 | | | |
| SARS2-R3- | GFTFSSYA (SEQ | ISYDGSNK (SEQ | ARGLSGTYRATTFDF (SEQ |
| G3-P1- | ID NO: 122) | ID NO: 1143) | ID NO: 1207) |
| F1_PelB- | | | |
| F_2020_Jun. | | | |
| 25_C04 | | | |
| SARS2-R3- | GGSFSSYH (SEQ | IDHYGSP (SEQ | ARGYAFDI (SEQ ID NO: |
| G1-P3- | ID NO: 1134) | ID NO: 1157) | 1208) |
| H10_PelB- | | | |
| F_2020_Jun. | | | |
| 25_H10 | | | |
| 85_PelB- | GYNFNDYW | IYPGDSDT (SEQ | ARGYAMDV (SEQ ID NO: |
| F_2020_Jun. | (SEQ ID NO: | ID NO: 135) | 1170) |
| 25_G10 | 1135) | | |
| SARS2-R3- | GYTFSNYD (SEQ | MNPNSGNT | ARGYGMDV (SEQ ID NO: |
| G1-P4- | ID NO: 1136) | (SEQ ID NO: | 1209) |
| A3_PelB- | | 1158) | |
| F_2020_Jun. | | | |
| 25_B06 | | | |
| SARS2-R3- | GYSFTNYW | IDPRDSYT (SEQ | ARHPAGGTSFDM (SEQ ID |
| G1-P3- | (SEQ ID NO: | ID NO: 1159) | NO: 1210) |
| H6_PelB- | 1137) | | |
| F_2020_Jun. | | | |
| 25_H08 | | | |
| SARS2-R3- | GYTFTNYW | TYPGDSDT (SEQ | ARRAGAQGAFDI (SEQ ID |
| G3-P1- | (SEQ ID NO: | ID NO: 135) | NO: 1211) |
| G5_PelB- | 1138) | | |
| F_2020_Jun. | | | |
| 25_H04 | | | |
| SARS2-R3- | GFTFENYN (SEQ | ITGHAYYT (SEQ | ARSNYASAGHGMDV (SEQ |
| G1-P4- | ID NO: 1139) | ID NO: 1160) | ID NO: 1212) |
| C10_PelB- | | | |
| F_2020_Jun. | | | |
| 25_F07 | | | |
| SARS2-R3- | GYTFTSYG (SEQ | ISAYNGNT (SEQ | ARTVGSLDY (SEQ ID NO: |
| G1-P1- | ID NO: 161) | ID NO: 141) | 1213) |
| F10_PelB- | | | |
| F_2020_Jun. | | | |
| 25_B04 | | | |
| SARS2-R3- | GGSISSGGYY | IYYSGST (SEQ | ARYDRDAFDI (SEQ ID NO: |
| T1-P4- | (SEQ ID NO: | ID NO: 1161) | 1214) |
| H4_PelB- | 1140) | | |
| F_2020_Jun. | | | |
| 20_H04 | | | |
| SARS2-R3- | GFTFSSYA (SEQ | ISYDGSNK (SEQ | ASGSYAQFDY (SEQ ID NO: |
| G2-P1- | ID NO: 122) | ID NO: 1143) | 1215) |
| E8_PelB- | | | |
| F_2020_Jun. | | | |
| 25_A02 | | | |
| SARS2-R3- | GFTFSSYG (SEQ | ISYDGSNK (SEQ | ATPLGFFGAFDI (SEQ ID |
| G1-P1- | ID NO: 1119) | ID NO: 1143) | NO: 1216) |
| C3_PelB- | | | |
| F_2020_Jun. | | | |
| 25_A07 | | | |
| SARS2-R3- | GFDFSNYD (SEQ | ITRDGGRT (SEQ | DKAGGE (SEQ ID NO: 1217) |
| G3-P1- | ID NO: 1141) | ID NO: 1162) | |
| G2_PelB- | | | |
| F_2020_Jun. | | | |
| 25_G04 | | | |
| SARS2-R3- | GGTFNNYA | IIPIVDIA (SEQ ID | VRGGSQGAYYMDV (SEQ |
| G1-P4- | (SEQ ID NO: | NO: 1156) | ID NO: 1218) |
| D1_PelB- | 1142) | | |
| F_2020_Jun. | | | |
| 25_A08 | | | |

TABLE 63B
Light chain (V_{L}) complementary determining regions (CDRs) of the COVID-19
antibodies

| Sequence ID | CDR1-IMGT | CDR2-IMGT | CDR3-IMGT |
|---|---|---|---|
| S1-R3-T1-H7 | SSNIGSNT | SNN | AAWDDSLNVHYV |
| (Ab12) | (SEQ ID NO: 227) | (SEQ ID NO: 228) | (SEQ ID NO: 229) |
| RBD-R3-E1- | SGSIASNY | EDN | QSFDSASLWV |
| G7 (Ab2-10) | (SEQ ID NO: 230) | (SEQ ID NO: 231) | (SEQ ID NO: 232) |
| S1-RBD-R3- | SSNIGSND | SNN | ATWDDSLSAGV |
| E1-D8 (Ab_3) | (SEQ ID NO: 233) | (SEQ ID NO: 234) | (SEQ ID NO: 235) |
| S1-RBD-R3- | SGSIASNY | QDK | QSYDSSSLWV |
| T1-C7 | (SEQ ID NO: 236) | (SEQ ID NO: 237) | (SEQ ID NO: 238) |
| RBD-R3-T1- | TGSIAGNY | DDN | QSYDSGNRGV |
| F4 (Ab2-2) | (SEQ ID NO: 239) | (SEQ ID NO: 240) | (SEQ ID NO: 241) |
| RBD-R3-E1- | TSNIGNNA | YNE | AAWDDSLSGHVV |
| A5 | (SEQ ID NO: 242) | (SEQ ID NO: 243) | (SEQ ID NO: 244) |
| S1-RBD-R3- | SGSIASNY | EDN | QSYDSSSLWV |
| T1-F5 | (SEQ ID NO: 245) | (SEQ ID NO: 246) | (SEQ ID NO: 247) |
| (Ab2-7) | | | |
| S1-R3-T1- | SSNIGSNS | SNN | AAWDDSLTGYV |
| A12 (Ab_5) | (SEQ ID NO: 248) | (SEQ ID NO: 249) | (SEQ ID NO: 250) |
| S1-R3-T1-A6 | SSDVGGYNY | EVS | AAWDDSLSGPV |
| (Ab_4) | (SEQ ID NO: 251) | (SEQ ID NO: 252) | (SEQ ID NO: 253) |
| S1-RBD-R3- | KIGSKS | DDS | HVWDSSSDQNV |
| T1-A5 | (SEQ ID NO: 254) | (SEQ ID NO: 255) | (SEQ ID NO: 256) |
| S1-RBD-R3- | SLRAYF | GQD | NSRDSGENHLI |
| T1-B3 (Ab_1) | (SEQ ID NO: 257) | (SEQ ID NO: 258) | (SEQ ID NO: 259) |
| S1-RBD-R3- | SSDVGTYNR | EVS | SSYTRTFTYV |
| T1-E7 | (SEQ ID NO: 260) | (SEQ ID NO: 261) | (SEQ ID NO: 262) |
| S1-RBD-R3- | DSDIGANF | RNT | QSYDSSLSAYV |
| T1-F9 | (SEQ ID NO: 263) | (SEQ ID NO: 264) | (SEQ ID NO: 265) |
| S1-R3-T1-C2 | SGSIARNY | ADR | QSYDSSNQAAV |
| (Ab_6) | (SEQ ID NO: 266) | (SEQ ID NO: 267) | (SEQ ID NO: 268) |
| RBD-R3-E1- | SLRSYY | DKD | NSRDRSDNHVV |
| B3 | (SEQ ID NO: 269) | (SEQ ID NO: 270) | (SEQ ID NO: 271) |
| RBD-R3-T1- | QSVSNN | DAT | QQYDNLPV |
| B5 | (SEQ ID NO: 272) | (SEQ ID NO: 273) | (SEQ ID NO: 274) |
| RBD-R3-T1- | SSDVGAYNY | DVT | AVWDDGLNGRVV |
| H3 (Ab_2) | (SEQ ID NO: 275) | (SEQ ID NO: 276) | (SEQ ID NO: 277) |
| S1-R3-T1-C4 | SNNVGNQG | MNN | SAWDSSLSRWV |
| (Ab_8) | (SEQ ID NO: 278) | (SEQ ID NO: 279) | (SEQ ID NO: 280) |
| S1-RBD-R3- | SGSIASNY | EDS | QSFHNSNPVI |
| T1-B12 | (SEQ ID NO: 281) | (SEQ ID NO: 282) | (SEQ ID NO: 283) |
| S1-RBD-R3- | SGSIASNY | EDN | QSYDSSNHWV |
| T1-G5 | (SEQ ID NO: 284) | (SEQ ID NO: 285) | (SEQ ID NO: 286) |
| S1-RBD-R3- | NIGSKS | EDR | QVWDGDSDHYV |
| T1-E2 (Ab7) | (SEQ ID NO: 287) | (SEQ ID NO: 288) | (SEQ ID NO: 289) |
| RBD-R3-T1- | SSDVGGYNL | EGS | SSYTITDVVV |
| F7 | (SEQ ID NO: 290) | (SEQ ID NO: 291) | (SEQ ID NO: 292) |
| S1-RBD-R3- | SSNIGSNP | SNN | AAWDDSLSGVV |
| T1-G1 | (SEQ ID NO: 293) | (SEQ ID NO: 294) | (SEQ ID NO: 295) |
| S1-RBD-R3- | SGSIASNY | EDN | QSYDSTNWV |
| T1-C2 | (SEQ ID NO: 296) | (SEQ ID NO: 297) | (SEQ ID NO: 298) |
| S1-RBD-R3- | NIGSES | EDR | QVWNPSGSLQYV |
| T1-H8 | (SEQ ID NO: 299) | (SEQ ID NO: 300) | (SEQ ID NO: 301) |
| S1-RBD-R3- | SGNIATNY | EDN | KSYDDGNHV |
| E1-E8 | (SEQ ID NO: 302) | (SEQ ID NO: 303) | (SEQ ID NO: 304) |
| RBD-R3-T1- | QSVSSN | DVS | QQRGVWPLT |
| H2 | (SEQ ID NO: 305) | (SEQ ID NO: 306) | (SEQ ID NO: 307) |
| S1-RBD-R3- | SGSIVSSY | EHN | QSYDSQNGV |
| T1-B7 | (SEQ ID NO: 308) | (SEQ ID NO: 309) | (SEQ ID NO: 310) |
| S1-RBD-R3- | SSDLGTYNY | DVF | SSYTSSSTYV |
| E1-E5 | (SEQ ID NO: 311) | (SEQ ID NO: 312) | (SEQ ID NO: 313) |
| S1-R3-T1-H6 | SIGTKS | DDD | QVWESDDDDLV |
| | (SEQ ID NO: 314) | (SEQ ID NO: 315) | (SEQ ID NO: 316) |
| S1-RBD-R3- | SLRRYY | GKN | NSRDISDNQWQWI |
| E1-C6 | (SEQ ID NO: 317) | (SEQ ID NO: 318) | (SEQ ID NO: 319) |
| S1-RBD-R3- | SSDVGGYKY | DVN | SSYTGRMNLYV |
| E1-F2 | (SEQ ID NO: 320) | (SEQ ID NO: 321) | (SEQ ID NO: 322) |
| S1-RBD-R3- | NIRTKG | YAS | QVWDSSSDLVV |
| T1-C3 | (SEQ ID NO: 323) | (SEQ ID NO: 324) | (SEQ ID NO: 325) |
| S1-RBD-R3- | SSDVGGYDY | DVS | SSYTSSSPVV |
| T1-G12 | (SEQ ID NO: 326) | (SEQ ID NO: 327) | (SEQ ID NO: 328) |
| S1-RBD-R3- | SRDVGSYDL | EGS | SSYTSSNSLV |
| E1-F1 | (SEQ ID NO: 329) | (SEQ ID NO: 330) | (SEQ ID NO: 331) |
| S1-RBD-R3- | GGSIASNY | EDN | QSYDSSNPVV |
| E1-H8 | (SEQ ID NO: 332) | (SEQ ID NO: 333) | (SEQ ID NO: 334) |
| S1-RBD-R3- | SSIGSND | SNN | AWDDSLSAVV |
| T1-F1 | (SEQ ID NO: 335) | (SEQ ID NO: 336) | (SEQ ID NO: 337) |
| S1-R3-T1- | SSNIGSNT | INN | AEWYDSLNVHYV |
| B10 | (SEQ ID NO: 338) | (SEQ ID NO: 339) | (SEQ ID NO: 340) |
| RBD-R3-E1- | SSNIGSNT | INN | AECYDSLNDHYV |
| D12 | (SEQ ID NO: 341) | (SEQ ID NO: 342) | (SEQ ID NO: 343) |
| RBD-R3-T1- | SSNIGSNT | SNN | AAWDDSLNVHYV |
| C5 | (SEQ ID NO: 344) | (SEQ ID NO: 345) | (SEQ ID NO: 346) |
| S1-RBD-R3- | SSNIGSNP | NNN | AAWDDSLNGL |
| T1-B4 | (SEQ ID NO: 347) | (SEQ ID NO: 348) | (SEQ ID NO: 349) |
| S1-RBD-R3- | SLRSYY | GKN | NSRDSSGDVRV |
| E1-E7 | (SEQ ID NO: 350) | (SEQ ID NO: 351) | (SEQ ID NO: 352) |
| S1-RBD-R3- | SSNIGSNP | DNN | EAWDDSLSGPV |
| T1-C8 | (SEQ ID NO: 353) | (SEQ ID NO: 354) | (SEQ ID NO: 355) |
| S1-RBD-R3- | SSNIGGNS | RNN | AAWDDSLNGWV |
| T1-D7 | (SEQ ID NO: 356) | (SEQ ID NO: 357) | (SEQ ID NO: 358) |
| RBD-R3-E1- | SGSIASNY | EDN | QSYDPNNHGVV |
| F5 | (SEQ ID NO: 359) | (SEQ ID NO: 360) | (SEQ ID NO: 361) |
| Ab_13 | SGSIASNY | EDN | QSYDSSNLWV |
| | (SEQ ID NO: 802) | (SEQ ID NO: 803) | (SEQ ID NO: 804) |
| Ab_14 | SSDVGAYNF | DFN | SSYAGSNNFDVV |
| | (SEQ ID NO: 805) | (SEQ ID NO: 806) | (SEQ ID NO: 807) |
| Ab_15 | SGTIASNY | EDN | QSYDTSNHYV |
| | (SEQ ID NO: 808) | (SEQ ID NO: 809) | (SEQ ID NO: 810) |
| Ab_16 | SSNIGAGYD | GTN | QSYDNSLTDPYV |
| | (SEQ ID NO: 811) | (SEQ ID NO: 812) | (SEQ ID NO: 813) |
| Ab_17 | SSDVGGSKY | DVT | AAWDDSLNGVV |
| | (SEQ ID NO: 814) | (SEQ ID NO: 815) | (SEQ ID NO: 816) |
| Ab_18 | SGSIANNF | EDN | QSYDSSNLV |
| | (SEQ ID NO: 817) | (SEQ ID NO: 818) | (SEQ ID NO: 819) |
| Ab_19 | SANIGSNA | GNT | AAWDDSLNGYV |
| | (SEQ ID NO: 820) | (SEQ ID NO: 821) | (SEQ ID NO: 822) |
| Ab_20 | SGSIASNY | EDN | QSYDSSNHWV |
| | (SEQ ID NO: 823) | (SEQ ID NO: 824) | (SEQ ID NO: 825) |
| Ab_21 | SSDVGGYDY | DVS | SSYTSSSPVV |
| | (SEQ ID NO: 826) | (SEQ ID NO: 827) | (SEQ ID NO: 828) |
| Ab_22 | TGSIAGNY | DDN | QSYDSGNRGV |
| | (SEQ ID NO: 829) | (SEQ ID NO: 830) | (SEQ ID NO: 831) |
| Ab_23 | SRDIGAYGY | EVR | SSYTSSSTLDVV |
| | (SEQ ID NO: 832) | (SEQ ID NO: 833) | (SEQ ID NO: 834) |
| Ab_24 | SSNIGRNA | SNN | SAWDTSLSTWV |
| | (SEQ ID NO: 835) | (SEQ ID NO: 836) | (SEQ ID NO: 837) |
| Ab_25 | SGSIASNY | EDN | QSYDSSNHWV |
| | (SEQ ID NO: 838) | (SEQ ID NO: 839) | (SEQ ID NO: 840) |
| Ab_26 | NIRTKG | YAS | QVWDSSSDLVV |
| | (SEQ ID NO: 841) | (SEQ ID NO: 842) | (SEQ ID NO: 843) |
| Ab_27 | SSDVGTYNR | EVS | SSYTRTFTYV |
| | (SEQ ID NO: 844) | (SEQ ID NO: 845) | (SEQ ID NO: 846) |
| Ab_28 | GGSIASNY | EDN | QSYDSSNPVV |
| | (SEQ ID NO: 847) | (SEQ ID NO: 848) | (SEQ ID NO: 849) |
| Ab_38 | QSVSSN | DVS | QQRGAWPLT |
| | (SEQ ID NO: 986) | (SEQ ID NO: 987) | (SEQ ID NO: 988) |
| WCS2-T4-E7 | SGDIATRH | ESN | QSYDSTNPWV |
| | (SEQ ID NO: 1219) | (SEQ ID NO: 1258) | (SEQ ID NO: 1285) |
| wcS2-E1-A9 | SSDVGAYNY | EVS | SSYAGTRKYYV |
| | (SEQ ID NO: 275) | (SEQ ID NO: 252) | (SEQ ID NO: 1286) |
| wcS2-T4-H8 | QIVTNNN | GAS | QQYYYWPLS |
| | (SEQ ID NO: 1220) | (SEQ ID NO: 1259) | (SEQ ID NO: 1287) |
| wcS2-T3-F5 | SSDVGGYKY | DVS | SSFSQTNSYV |
| | (SEQ ID NO: 320) | (SEQ ID NO: 306) | (SEQ ID NO: 1288) |
| wcS2-E2-C1 | SSDVGRYKY | EVN | SSYAGSNNPYV |
| | (SEQ ID NO: 1221) | (SEQ ID NO: 1260) | (SEQ ID NO: 1289) |
| wcS2-T4-C9 | QNVPSNS | GAS | QLYDRSSQLA |
| | (SEQ ID NO: 1222) | (SEQ ID NO: 1259) | (SEQ ID NO: 1290) |
| wcS2-T3-B9 | SLGTYY | GKN | NSRNNSGYHE |
| | (SEQ ID NO: 1223) | (SEQ ID NO: 318) | (SEQ ID NO: 1291) |
| wcS2-E3-C8 | QSNIGSNT | VNN | SSYAGSNDYV |
| | (SEQ ID NO: 1224) | (SEQ ID NO: 1261) | (SEQ ID NO: 1292) |
| wcS2-T4-F8 | SGSIASNY | GDN | QSFDGSYHWV |
| | (SEQ ID NO: 230) | (SEQ ID NO: 1262) | (SEQ ID NO: 1293) |
| wcS2-T3-F1 | SSNIGSNT | SNN | ATWDDGLSGRV |
| | (SEQ ID NO: 227) | (SEQ ID NO: 228) | (SEQ ID NO: 1294) |
| wcS2-E2-B1 | SLRSYY | GKN | NSRDSSGNHLRV |
| | (SEQ ID NO: 269) | (SEQ ID NO: 318) | (SEQ ID NO: 1295) |
| wcS2-T2-G3 | SNNVGNQG | RNN | SAWDNSLGAWV |
| | (SEQ ID NO: 278) | (SEQ ID NO: 357) | (SEQ ID NO: 1296) |
| wcS2-T1-A6 | QSNIGSNT | VNN | SSYAGSNDYV |
| | (SEQ ID NO: 1224) | (SEQ ID NO: 1261) | (SEQ ID NO: 1292) |
| wcS2-T4-D4 | SGDIATRH | ESN | LTYDITNPWV |
| | (SEQ ID NO: 1219) | (SEQ ID NO: 1258) | (SEQ ID NO: 1297) |
| wcS2-T2-D10 | SGDIATKH | DND | SYDDSTNPCV |
| | (SEQ ID NO: 1225) | (SEQ ID NO: 1263) | (SEQ ID NO: 1298) |
| wcS2-T1-G9 | QSISSSY | DAS | QQRGNWPLT |
| | (SEQ ID NO: 1226) | (SEQ ID NO: 1264) | (SEQ ID NO: 1299) |
| wcS2-E3-H7 | QNVPSNS | GAS | QLYDRSSQLA |
| | (SEQ ID NO: 1222) | (SEQ ID NO: 1259) | (SEQ ID NO: 1290) |
| wcS2-T2-C11 | SLTIFF | KDT | QSADTSGTLKV |
| | (SEQ ID NO: 1227) | (SEQ ID NO: 1265) | (SEQ ID NO: 1300) |
| SARS2-R3- | SSDLGGHNF (SEQ | GVN (SEQ ID NO: | SSYEATHIYV (SEQ ID |
| G2-P1- | ID NO: 1228) | 1266) | NO: 1301) |
| H9_PelB- | | | |
| F_2020_Jun. | | | |
| 19_D08 | | | |
| SARS2-R3- | SSDVGSYNL (SEQ | EVS (SEQ ID NO: | SSFSSGSIPYV (SEQ ID |
| G3-P1- | ID NO: 1229) | 252) | NO: 1302) |
| F8_PelB- | | | |
| F_2020_Jun. | | | |
| 19_F11 | | | |
| SARS2-R3- | QSVSTN (SEQ ID | GAS (SEQ ID NO: | QHYANWPRT (SEQ ID |
| G2-P1- | NO: 1230) | 1259) | NO: 1303) |
| B4_PelB- | | | |
| F_2020_Jun. | | | |
| 19_B02 | | | |
| SARS2-R3- | SSNIGSNT (SEQ ID | SNN (SEQ ID NO: | ATWDDSLNGPV (SEQ |
| G3-P1- | NO: 227) | 228) | ID NO: 1304) |
| B2_PelB- | | | |
| F_2020_Jun. | | | |
| 19_A08 | | | |
| SARS2-R3- | NIGSKS (SEQ ID | DDT (SEQ ID NO: | QVWDTSSDHPYV |
| G3-P1- | NO: 287) | 1267) | (SEQ ID NO: 1305) |
| B3_PelB- | | | |
| F_2020_Jun. | | | |
| 19_B08 | | | |
| SARS2-R3- | SLRSYY (SEQ ID | GGN (SEQ ID NO: | SSRATSAFYV (SEQ ID |
| G1-P3- | NO: 269) | 1268) | NO: 1306) |
| E8_PelB- | | | |
| F_2020_Jun. | | | |
| 19_H04 | | | |
| SARS2-R3- | SSNIGAGYD (SEQ | GNS (SEQ ID NO: | QSYDSSLSGWV (SEQ |
| G1-P2- | ID NO: 811) | 1269) | ID NO: 1307) |
| D7_PelB- | | | |
| F_2020_Jun. | | | |
| 19_H09 | | | |
| SARS2-R3- | ALPNRY (SEQ ID | KDS (SEQ ID NO: | QSVNTIGTYV (SEQ ID |
| G1-P1- | NO: 1231) | 1270) | NO: 1308) |
| C6_PelB- | | | |
| F_2020_Jun. | | | |
| 19_H02 | | | |
| SARS2-R3-T- | SSDLVGHKF (SEQ | EIN (SEQ ID NO: | TCYDEDHIYV (SEQ ID |
| P2-E12_PelB- | ID NO: 1232) | 1271) | NO: 1309) |
| F_2020_Jun. | | | |
| 19_D07 | | | |
| SARS2-R3- | ENVNNW (SEQ ID | KAS (SEQ ID NO: | QQYKSYS (SEQ ID |
| G2-P1- | NO: 1233) | 1272) | NO: 1310) |
| D12_PelB- | | | |
| F_2020_Jun. | | | |
| 19_H04 | | | |
| SARS2-R3- | SGSIASNY (SEQ ID | AHN (SEQ ID NO: | HSYDTNNPVV (SEQ ID |
| G1-P2- | NO: 230) | 1273) | NO: 1311) |
| E8_PelB- | | | |
| F_2020_Jun. | | | |
| 19_E10 | | | |
| SARS2-R3- | QSVRSN (SEQ ID | GVS (SEQ ID NO: | QHYGSSPLYT (SEQ ID |
| G1-P1- | NO: 1234) | 1274) | NO: 1312) |
| A10_PelB- | | | |
| F_2020_Jun. | | | |
| 19_H01 | | | |
| SARS2-R3- | SSDVGSYNR (SEQ | EVS (SEQ ID NO: | SSYTSSSPYV (SEQ ID |
| G1-P3- | ID NO: 1235) | 252) | NO: 1313) |
| B5_PelB- | | | |
| F_2020_Jun. | | | |
| 19_D02 | | | |
| SARS2-R3- | IGDIARNY (SEQ | EDD (SEQ ID NO: | QSYDTTIV (SEQ ID |
| G3-P1- | ID NO: 1236) | 1275) | NO: 1314) |
| A2_PelB- | | | |
| F_2020_Jun. | | | |
| 19_B07 | | | |
| SARS2-R3- | SSNIGAGYH (SEQ | GNQ (SEQ ID NO: | SAWDDSLGGEV (SEQ |
| G3-P1- | ID NO: 1237) | 1276) | ID NO: 1315) |
| D1_PelB- | | | |
| F_2020_Jun. | | | |
| 19_G09 | | | |
| SARS2-R3- | SNNVGHEG (SEQ | KND (SEQ ID NO: | SAWDSSLGSWV (SEQ |
| G1-P2- | ID NO: 1238) | 1277) | ID NO: 1316) |
| D4_PelB- | | | |
| F_2020_Jun. | | | |
| 19_G09 | | | |
| SARS2-R3- | RSNIGSNT (SEQ ID | SNN (SEQ ID NO: | QSYDSSVV (SEQ ID |
| G1-P1- | NO: 1239) | 228) | NO: 1317) |
| B6_PelB- | | | |
| F_2020_Jun. | | | |
| 19_E02 | | | |
| SARS2-R3- | NLGEIY (SEQ ID | QDK (SEQ ID NO: | QAWDSSTGV (SEQ ID |
| G3-P1- | NO: 1240) | 237) | NO: 1318) |
| G8_PelB- | | | |
| F_2020_Jun. | | | |
| 25_A05 | | | |
| SARS2-R3- | ALPDKY (SEQ ID | KDS (SEQ ID NO: | QSADSSGTWV (SEQ |
| G1-P1- | NO: 1241) | 1270) | ID NO: 1319) |
| F11_PelB- | | | |
| F_2020_Jun. | | | |
| 25_B05 | | | |
| SARS2-R3- | SSDVGGYNH (SEQ | DVS (SEQ ID NO: | TSY AGSNSLV (SEQ ID |
| T1-P3- | ID NO: 1242) | 306) | NO: 1320) |
| F4_PelB- | | | |
| F_2020_Jun. | | | |
| 20_C03 | | | |
| SARS2-R3- | SGDVGGYNS (SEQ | EVT (SEQ ID NO: | SSYAGSNNYV (SEQ ID |
| G1-P2- | ID NO: 1243) | 1278) | NO: 1321) |
| B8_PelB- | | | |
| F_2020_Jun. | | | |
| 25_C06 | | | |
| SARS2-R3- | SGSIASNY (SEQ ID | EDN (SEQ ID NO: | QSYDSPWV (SEQ ID |
| G1-P2- | NO: 230) | 231) | NO: 1322) |
| D3_PelB- | | | |
| F_2020_Jun. | | | |
| 25_C12 | | | |
| SARS2-R3- | SSNIGSNT (SEQ ID | SNN (SEQ ID NO: | AVWDDSLNGLV (SEQ |
| G1-P4- | NO: 227) | 228) | ID NO: 1323) |
| C5_PelB- | | | |
| F_2020_Jun. | | | |
| 25_C07 | | | |
| SARS2-R3- | SNNVGHEG (SEQ | RNK (SEQ ID NO: | SAWDGRLNGYL (SEQ |
| G1-P2- | ID NO: 1238) | 1279) | ID NO: 1324) |
| D6_PelB- | | | |
| F_2020_Jun. | | | |
| 25_D02 | | | |
| SARS2-R3- | KLEDKF (SEQ ID | EDA (SEQ ID NO: | QAWAITTEV (SEQ ID |
| G1-P1- | NO: 1244) | 1280) | NO: 1325) |
| B7_PelB- | | | |
| F_2020_Jun. | | | |
| 25_A03 | | | |
| 95_PelB- | QSVSSTY (SEQ ID | GAS (SEQ ID NO: | QHSYRTPYT (SEQ ID |
| F_2020_Jun. | NO: 1245) | 1259) | NO: 1326) |
| 25_G11 | | | |
| 73_PelB- | QSVRDGY (SEQ ID | GAS (SEQ ID NO: | QQRSNWPPT (SEQ ID |
| F_2020_Jun. | NO: 1246) | 1259) | NO: 1327) |
| 25_F09 | | | |
| SARS2-R3- | SDDVGGFAH (SEQ | DVS (SEQ ID NO: | SSYAGRNGVI (SEQ ID |
| G3-P1- | ID NO: 1247) | 306) | NO: 1328) |
| G9_PelB- | | | |
| F_2020_Jun. | | | |
| 25_B05 | | | |
| SARS2-R3- | SNNVGDQG (SEQ | RNN (SEQ ID NO: | SAWDSSLSAQV (SEQ |
| G1-P3- | ID NO: 1248) | 357) | ID NO: 1329) |
| G7_PelB- | | | |
| F_2020_Jun. | | | |
| 25_H01 | | | |
| SARS2-R3- | SGSIASNY (SEQ ID | EDN (SEQ ID NO: | QSYDNNNHVV (SEQ |
| G1-P4- | NO: 230) | 231) | ID NO: 1330) |
| A2_PelB- | | | |
| F_2020_Jun. | | | |
| 25_A06 | | | |
| SARS2-R3- | SSNIGAGYD (SEQ | GNS (SEQ ID NO: | QSYDSSLSGWV (SEQ |
| G3-P1- | ID NO: 811) | 1269) | ID NO: 1307) |
| G1_PelB- | | | |
| F_2020_Jun. | | | |
| 25_F04 | | | |
| SARS2-R3- | SSDLGGHNF (SEQ | DVY (SEQ ID NO: | SSYAGSNPYV (SEQ ID |
| G3-P1- | ID NO: 1228) | 1281) | NO: 1331) |
| F1_PelB- | | | |
| F_2020_Jun. | | | |
| 25_C04 | | | |
| SARS2-R3- | RSNIGINS (SEQ ID | RNN (SEQ ID NO: | AAWDGSLNGPL (SEQ |
| G1-P3- | NO: 1249) | 357) | ID NO: 1332) |
| H10_PelB- | | | |
| F_2020_Jun. | | | |
| 25_H10 | | | |
| 85_PelB- | SSNIGNYG (SEQ | SNN (SEQ ID NO: | AAWDDSLKGV (SEQ |
| F_2020_Jun. | ID NO: 1250) | 228) | ID NO: 1333) |
| 25_G10 | | | |
| SARS2-R3- | TSNIGRNK (SEQ | LNN (SEQ ID NO: | AAWDDSLTGYV (SEQ |
| G1-P4- | ID NO: 1251) | 1282) | ID NO: 250) |
| A3_PelB- | | | |
| F_2020_Jun. | | | |
| 25_B06 | | | |
| SARS2-R3- | SLSRYY (SEQ ID | GKD (SEQ ID NO: | NSRDSSGNL (SEQ ID |
| G1-P3- | NO: 1252) | 1283) | NO: 1334) |
| H6_PelB- | | | |
| F_2020_Jun. | | | |
| 25_H08 | | | |
| SARS2-R3- | SSDVGGYNY (SEQ | DVN (SEQ ID NO: | SSYTSSSTYV (SEQ ID |
| G3-P1- | ID NO: 251) | 321) | NO: 313) |
| G5_PelB- | | | |
| F_2020_Jun. | | | |
| 25_H04 | | | |
| SARS2-R3- | GLPKHY (SEQ ID | KDT (SEQ ID NO: | QSGDTSGPVV (SEQ ID |
| G1-P4- | NO: 1253) | 1265) | NO: 1335) |
| C10_PelB- | | | |
| F_2020_Jun. | | | |
| 25_F07 | | | |
| SARS2-R3- | SSNIGRGYN (SEQ | GNS (SEQ ID NO: | SAWDDSLNNVV (SEQ |
| G1-P1- | ID NO: 1254) | 1269) | ID NO: 1336) |
| F10_PelB- | | | |
| F_2020_Jun. | | | |
| 25_B04 | | | |
| SARS2-R3- | NIGHKG (SEQ ID | DDS (SEQ ID NO: | QVWESYGDHVV (SEQ |
| T1-P4- | NO: 1255) | 255) | ID NO: 1337) |
| H4_PelB- | | | |
| F_2020_Jun. | | | |
| 20_H04 | | | |
| SARS2-R3- | QSLLHSNGYNY | FGS (SEQ ID NO: | MQVLQTPPIT (SEQ ID |
| G2-P1- | (SEQ ID NO: 1256) | 1284) | NO: 1338) |
| E8_PelB- | | | |
| F_2020_Jun. | | | |
| 25_A02 | | | |
| SARS2-R3- | TSNIGAGYD (SEQ | DNN (SEQ ID NO: | QSYDSGLDGSV (SEQ |
| G1-P1- | ID NO: 1257) | 354) | ID NO: 1339) |
| C3_PelB- | | | |
| F_2020_Jun. | | | |
| 25_A07 | | | |
| SARS2-R3- | SSDVGGYNH (SEQ | DVS (SEQ ID NO: | TSYAGSNSLV (SEQ ID |
| G3-P1- | ID NO: 1242) | 306) | NO: 1320) |
| G2_PelB- | | | |
| F_2020_Jun. | | | |
| 25_G04 | | | |
| SARS2-R3- | SGSIASNY (SEQ ID | EDD (SEQ ID NO: | QSYDNNNAVI (SEQ ID |
| G1-P4- | NO: 230) | 1275) | NO: 1340) |
| D1_PelB- | | | |
| F_2020_Jun. | | | |
| 25_A08 | | | |

The amino acid sequences of the heavy and light chain framework regions of the COVID-19 antibodies are shown in Table 64A-B below:

TABLE 64A
Heavy chain (V_{H}) framework regions (FRs) of the COVID-19 antibodies.

| Seq ID | FR1-IMGT | FR2-IMGT | FR3-IMGT | FR4-IMGT |
|---|---|---|---|---|
| S1-R3- | EVQLVESGPGV | WNWIRQPPGKPL | NKNPSLK.SRVTIS | WSRGTL |
| T1-H7 | VSPSATLFLTCS | EWIGF | SDTSKNEFSLTLT | VTVSS |
| (Ab12) | VS | (SEQ ID NO: 363) | SVTAADTAVYYC | (SEQ ID |
| | (SEQ ID NO: 362) | | (SEQ ID NO: 364) | NO: 365) |
| RBD-R3- | EVQLVESGPGL | WSWIRQPAGKG | NYNPSLKSRVTM | WGQGTL |
| E1-G7 | VKPSETLSLTCT | LEWIGR | SVDTSKNQFSLKL | VTVSS |
| (Ab2-10) | VS | (SEQ ID NO: 367) | SSVTAADTAVYY | (SEQ ID |
| | (SEQ ID NO: 366) | | C | NO: 369) |
| | | | (SEQ ID NO: 368) | |
| S1-RBD- | EVQLVESGPGL | WNWIR*PPGKPL | NKNPSLKSRVSIS | WSRGTL |
| R3-E1- | VKPSATLFLTCS | EWIGF | SDPSKNEFSLTLT | VTVSS |
| D8 | VS | (SEQ ID NO: 371) | SVTAADTAVYYC | (SEQ ID |
| (Ab_3) | (SEQ ID NO: 370) | | (SEQ ID NO: 372) | NO: 373) |
| S1-RBD- | QVQLQQSGPGL | WNWIRQSPSRGL | DYAVSVKSRITIN | WGQGTT |
| R3-T1- | VKPSQTLSLTCA | EWLGR | PDTSKNQFSLQLS | VTVSS |
| C7 | IS | (SEQ ID NO: 375) | SVTPEDTAVYYC | (SEQ ID |
| | (SEQ ID NO: 374) | | (SEQ ID NO: 376) | NO: 377) |
| RBD-R3- | QVQLVQSGGGV | MHWVRQAPGK | YYADSVKGRFTIS | WGQGTT |
| T1-F4 | VQPGRSLRLSC | GLEWLAV | RDKSKNTLYLQIN | VTVSS |
| (Ab2-2) | AAS | (SEQ ID NO: 379) | SLRAEDTAVYYC | (SEQ ID |
| | (SEQ ID NO: 378) | | (SEQ ID NO: 380) | NO: 381) |
| RBD-R3- | QVQLVQSGG.G | IHWVRQAPGKG | YYGDSVKGRFTIS | WGQGTT |
| E1-A5 | VVQPGKSLRLS | LEWVGV | RDNAKNSLYLQM | VTVSS |
| | CTAS | (SEQ ID NO: 383) | NSLRVEDTAVYY | (SEQ ID |
| | (SEQ ID NO: 382) | | C | NO: 385) |
| | | | (SEQ ID NO: 384) | |
| S1-RBD- | QVTLKESGPTR | VGWIRQPPGKAL | RYSPSLKSRLTIT | WGQGTT |
| R3-T1- | VKPTQTLTLTCT | EWLAL | KDTSKNQVVLTM | VTVSS |
| F5 | FS | (SEQ ID NO: 387) | TNMDPVDTATYY | (SEQ ID |
| (Ab2-7) | (SEQ ID NO: 386) | | C | NO: 389) |
| | | | (SEQ ID NO: 388) | |
| S1-R3- | QVQLVQSGGGV | MHWVRQAPGK | NYADFVEGRFTIS | WGQGTL |
| T1-A12 | VQPGRSLRLSC | GLEWVAV | RDNSKNTLYLQM | VTVSS |
| (Ab_5) | AAS | (SEQ ID NO: 391) | NSLRPEDTGVYY | (SEQ ID |
| | (SEQ ID NO: 390) | | C | NO: 393) |
| | | | (SEQ ID NO: 392) | |
| S1-R3- | QVQLVQSGGGL | MHWVRQAPGK | PYADSVKGRFTIS | WGQGTL |
| T1-A6 | VQPGRSLRLSC | GLEWVAA | RDNAKNSLYLQM | VTVSS |
| (Ab_4) | AAS | (SEQ ID NO: 395) | NSLRDEDTAVYY | (SEQ ID |
| | (SEQ ID NO: 394) | | C | NO: 397) |
| | | | (SEQ ID NO: 396) | |
| S1-RBD- | QVQLVQSGAEV | IHWVRQAPGQG | NFAQRFQGRVTM | WGQGTL |
| R3-T1- | KKPGASVKFSC | LEWMGW | TTDTSVSTAYMD | VTVSS |
| A5 | KAS | (SEQ ID NO: 399) | LRRLRSDDTAVY | (SEQ ID |
| | (SEQ ID NO: 398) | | YC | NO: 401) |
| | | | (SEQ ID NO: 400) | |
| S1-RBD- | QVHLVQSGGGV | MHWVRQAPGK | YYAESVKGRFTIS | WGQGTM |
| R3-T1- | VQPGRSLRLSC | GLEWVAL | RDNSKNTLYLQM | VTVSS |
| B3 | AAS | (SEQ ID NO: 403) | NSLRAEDTAVYY | (SEQ ID |
| (Ab_1) | (SEQ ID NO: 402) | | C | NO: 405) |
| | | | (SEQ ID NO: 404) | |
| S1-RBD- | QVQLVQSGTEV | LHWVRQAPGQG | NYAQKFQGRVTL | WGQGTL |
| R3-T1- | KKPGASVKVSC | LEWMGW | TRDTSISTAYMEL | VTVSS |
| E7 | KAS | (SEQ ID NO: 407) | SGLRSDDTAVYY | (SEQ ID |
| | (SEQ ID NO: 406) | | C | NO: 409) |
| | | | (SEQ ID NO: 408) | |
| S1-RBD- | QVTLKESGPTL | VSWIRQPPGKAL | FYSTALKTRLTIS | WGRGTL |
| R3-T1- | VKPTQTLTLTCT | EWLAR | KDTSKNQVVFTM | VTVSS |
| F9 | LS | (SEQ ID NO: 411) | TSMDPVDTATYY | (SEQ ID |
| | (SEQ ID NO: 410) | | C | NO: 413) |
| | | | (SEQ ID NO: 412) | |
| S1-R3- | QVQLVQSGGGV | FHWVRQAPGKG | LYADSVKGRFTIS | WGQGTT |
| T1-C2 | VQPGRSLRLSC | LQWVAV | RDDSQNMLYLEM | VTVSS |
| (Ab_6) | AAS | (SEQ ID NO: 415) | HSLRLEDTAVYY | (SEQ ID |
| | (SEQ ID NO: 414) | | C | NO: 417) |
| | | | (SEQ ID NO: 416) | |
| RBD-R3- | QVQLVQSGAEV | IGWVRQMPGKG | RYSPSFQGQVTIS | WGQGTL |
| E1-B3 | KKPGESLKISCK | LEWMGI | ADKSISTAYLQW | VTVSS |
| | GS | (SEQ ID NO: 419) | SSLKASDTAMYY | (SEQ ID |
| | (SEQ ID NO: 418) | | C | NO: 421) |
| | | | (SEQ ID NO: 420) | |
| RBD-R3- | QVQL*QSGPGL | WSWIRQSPSRGL | DFAQSVRSRIVIN | WGQGTL |
| T1-B5 | VKASQTLSLTC | EWLAR | PDTSKNHVYLQL | VTVSS |
| | VIS | (SEQ ID NO: 423) | RSVTPEDTAVYY | (SEQ ID |
| | (SEQ ID NO: 422) | | C | NO: 425) |
| | | | (SEQ ID NO: 424) | |
| RBD-R3- | EVQLVQSGAEV | ISWVRQAPGQGL | NYAQKLQGRVT | WGKGTL |
| T1-H3 | KKPGSSVKVSC | EWMGW | MTTDTSTSTAYM | VTVSS |
| (Ab_2) | KTS | (SEQ ID NO: 427) | ELRSLRSDDTAV | (SEQ ID |
| | (SEQ ID NO: 426) | | YYC | NO: 429) |
| | | | (SEQ ID NO: 428) | |
| S1-R3- | QVQLVQSGAEV | ISWLRQAPGQGL | NYAQKFQGRVT | WGQGTL |
| T1-C4 | KKPGSSVKVSC | EWMGW | MTRDTSISTAYM | VTVSS |
| (Ab_8) | KAS | (SEQ ID NO: 431) | ELSRLRSDDTAV | (SEQ ID |
| | (SEQ ID NO: 430) | | YYC | NO: 433) |
| | | | (SEQ ID NO: 432) | |
| S1-RBD- | QVQLVQSGAEV | ISWVRQAPGQGL | NYAQKFQDRVAI | WGQGTL |
| R3-T1- | KKPGSSVKVSC | EWMGG | TADKSTSTAYME | VTVSS |
| B12 | KAS | (SEQ ID NO: 435) | LSSLRSEDTAVYY | (SEQ ID |
| | (SEQ ID NO: 434) | | C | NO: 437) |
| | | | (SEQ ID NO: 436) | |
| S1-RBD- | QVQLVQSGGGL | MSWVRQAPGKG | YYVDSVKGRFTIS | WGQGTT |
| R3-T1- | VQPGGSLRLSC | LEWVAN | RDNAKNSLYLQM | VTVSS |
| G5 5 | AAS | (SEQ ID NO: 439) | NSLRAEDTAVYY | (SEQ ID |
| | (SEQ ID NO: 438) | | C | NO: 441) |
| | | | (SEQ ID NO: 440) | |
| S1-RBD- | QVQLVQSGGGL | MHWVRQVPGK | GYADSVKGRFTIS | WGQGTM |
| R3-T1- | VQPGRSLRLSC | GLEWVSG | RDNAKNSLYLQM | VTVSS |
| E2 (Ab7) | AAS | (SEQ ID NO: 443) | NSLRAEDTALYY | (SEQ ID |
| | (SEQ ID NO: 442) | | C | NO: 445) |
| | | | (SEQ ID NO: 444) | |
| RBD-R3- | QVQLVQSGGGL | MHWVRQVPGK | GYADSVKGRFTIS | WGQGTT |
| T1-F7 | VQPGRSLRLSC | GLEWVSG | RDNVKNSLYLQM | VTVSS |
| | AAS | (SEQ ID NO: 447) | TSLRAEDTAVYF | (SEQ ID |
| | (SEQ ID NO: 446) | | C | NO: 449) |
| | | | (SEQ ID NO: 448) | |
| S1-RBD- | EVQLVQSGAEV | IGWVRQMPGKG | KYSPSFQGQVTIS | WGQGTL |
| R3-T1- | KKPGESLRISCK | LEWMGS | ADRSISTAYLQWS | VTVSS |
| G1 | AS | (SEQ ID NO: 451) | GLKASDTAMYYC | (SEQ ID |
| | (SEQ ID NO: 450) | | (SEQ ID NO: 452) | NO: 453) |
| S1-RBD- | EVQLVQSGAEV | ISWVRQAPGQGL | KYAQKLQGRVT | WGQGTL |
| R3-T1- | KKPGASVKVSC | EWMGW | MTTDTSTSTAYM | VTVSS |
| C2 | KAS | (SEQ ID NO: 455) | ELRSLRSDDTAV | (SEQ ID |
| | (SEQ ID NO: 454) | | YYC | NO: 457) |
| | | | (SEQ ID NO: 456) | |
| S1-RBD- | EVQLVQSGAEV | ISWVRQAPGQGL | RYAQKFQGRVTL | WGQGTM |
| R3-T1- | EKPGSSVKVSC | EWMGW | TIDTSSSTAYMEL | VTVSS |
| H8 | KAS | (SEQ ID NO: 459) | SSLRSEDTAVYYC | (SEQ ID |
| | (SEQ ID NO: 458) | | (SEQ ID NO: 460) | NO: 461) |
| S1-RBD- | QVQLVQSGAEV | ISWVRQAPGQGL | NYAQKLQGRVT | WGQGTL |
| R3-E1- | KKPGASVKVSC | EWMGW | MTTDTSTSTAYM | VTVSS |
| E8 | KAS | (SEQ ID NO: 463) | EVRSLRSDDTAV | (SEQ ID |
| | (SEQ ID NO: 462) | | YYC | NO: 465) |
| | | | (SEQ ID NO: 464) | |
| RBD-R3- | QVTLKESGPTL | VGWIRQPPGKAL | RYSPSLRSRLTITR | WGQGTL |
| T1-H2 | VKPTQTLTLTCT | EWLAL | DTSKNQVVLTVT | VTVSS |
| | FS | (SEQ ID NO: 467) | DMDPADTGTYYC | (SEQ ID |
| | (SEQ ID NO: 466) | | (SEQ ID NO: 468) | NO: 469) |
| S1-RBD- | QVTLKESGPAL | VSWIRQSPGKAL | YYTTSLKTRLTIT | WGQGTL |
| R3-T1- | VKSTQTLTLTCT | EWLAL | KDTSKNQVVLTM | VTVSS |
| B7 | IS | (SEQ ID NO: 471) | TSMDPLDTATYY | (SEQ ID |
| | (SEQ ID NO: 470) | | C | NO: 473) |
| | | | (SEQ ID NO: 472) | |
| S1-RBD- | QVQLVQSGGGL | MTWFRQAPGKG | KYADSVKGRFTIS | WGQGTL |
| R3-E1- | VKPGGSLRLSC | LEWISY | RDNAKNSLYLQM | VTVSS |
| E5 | AAS | (SEQ ID NO: 475) | DSLRAEDTAVYY | (SEQ ID |
| | (SEQ ID NO: 474) | | C | NO: 477) |
| | | | (SEQ ID NO: 476) | |
| S1-R3- | QVQLVQSGGGV | VHWVRQAPGKG | YYADSVKGRFTL | WGHGTL |
| T1-H6 | VQPGKSLRLSC | LEWVAV | SRDNSKNTVYLQ | VTVSS |
| | AAS | (SEQ ID NO: 479) | LSSLRREDTAVY | (SEQ ID |
| | (SEQ ID NO: 478) | | YC | NO: 481) |
| | | | (SEQ ID NO: 480) | |
| S1-RBD- | QVQLVQSGGGV | MHWVRQAPGK | YYADSVKGRFTIS | WGQGTM |
| R3-E1- | VQPGRSLRLSC | GLEYVSA | RDNGKNSLYLQM | VTVSS |
| C6 | AAS | (SEQ ID NO: 483) | SSLRAEDTAVYY | (SEQ ID |
| | (SEQ ID NO: 482) | | C | NO: 485) |
| | | | (SEQ ID NO: 484) | |
| S1-RBD- | QVQLVQSGGGL | MSWVRQAPGKG | YYVDSVKGRFTIS | WGQGTT |
| R3-E1- | VQPGGSLRLSC | LEWVAN | RDNAKNSLYLQM | VTVSS |
| F2 | AAS | (SEQ ID NO: 487) | NGLRAEDTAVYY | (SEQ ID |
| | (SEQ ID NO: 486) | | C | NO: 489) |
| | | | (SEQ ID NO: 488) | |
| S1-RBD- | QVQLVQSGGGL | MHWVRQVPGK | GYADSVKGRFTIS | WGQGTM |
| R3-T1- | VQPGRSLRLSC | GLEWVSG | RDNAKNSLYLQM | VTVSS |
| C3 | AAS | (SEQ ID NO: 491) | NSLRAEDTALYY | (SEQ ID |
| | (SEQ ID NO: 490) | | C | NO: 493) |
| | | | (SEQ ID NO: 492) | |
| S1-RBD- | QVQLVQSGGGL | MHWVRQAPGK | GYVDSVKGRFTIS | WGLGTL |
| R3-T1- | VQPGGSLRLSC | GLEWVSG | RDNRNNKVYLQ | VTVSS |
| G12 | ATS | (SEQ ID NO: 495) | MNNLRAEDTAVY | (SEQ ID |
| | (SEQ ID NO: 494) | | YC | NO: 497) |
| | | | (SEQ ID NO: 496) | |
| S1-RBD- | EVQLVESGPGL | WSWVRQPPGKG | NYNPSLKSRVTIS | WGQGTM |
| R3-E1- | VKPSGTLSLTCA | LEWIGE | VDTSKNQFSLKLS | VTVSS |
| F1 | VS | (SEQ ID NO: 499) | SVTAADTAVYYC | (SEQ ID |
| | (SEQ ID NO: 498) | | (SEQ ID NO: 500) | NO: 501) |
| S1-RBD- | QVTLKESGPGL | WSWIRQSPGKGL | IYNPSLKSRLTMSI | WGQGTL |
| R3-E1- | VNPSETLSLTCT | EWIGY | DTSKNQLSLNLRS | VTVSS |
| H8 | VS | (SEQ ID NO: 503) | LTAADTAVYYC | (SEQ ID |
| | (SEQ ID NO: 502) | | (SEQ ID NO: 504) | NO: 505) |
| S1-RBD- | EVQLVESGPGL | WNWIRQTPGKP | NKNPPLLSRVSISS | WSGGTL |
| R3-T1- | MKPSETLFLSCS | LEWMGI | DPSNNEFSLTLTS | ATVSS |
| F1 | VS | (SEQ ID NO: 507) | VTAADTAVYYC | (SEQ ID |
| | (SEQ ID NO: 506) | | (SEQ ID NO: 508) | NO: 509) |
| S1-R3- | EVQLVESGPGL | WNWIRQAPGKP | NKNPSLKSRVTIS | WSRGTL |
| T1-B10 | VSPSATLFLTCS | LEWMGV | SETSDNKFSLTLT | GTVSS |
| | VS | (SEQ ID NO: 511) | SVTAEDTAVYYC | (SEQ ID |
| | (SEQ ID NO: 510) | | (SEQ ID NO: 512) | NO: 513) |
| RBD-R3- | EVQLVESGPGV | WNWIRQPPGKPL | NKNPSLKSRVTIS | WSRGTL |
| E1-D12 | VSPSATLFLTCS | EWIGV | SKTSDNKFSLTLT | GTVSS |
| | VS | (SEQ ID NO: 515) | SVTAEDTAVYYC | (SEQ ID |
| | (SEQ ID NO: 514) | | (SEQ ID NO: 516) | NO: 517) |
| RBD-R3- | EVQLVESGPGV | WNWIRQAPGKA | NNNPSLKSRVTIS | WSRGTL |
| T1-C5 | VSPSATLFLTCS | LEWIGF | SDTSKNEFSLTLT | VTVSS |
| | VS | (SEQ ID NO: 519) | SVTAADTAVYYC | (SEQ ID |
| | (SEQ ID NO: 518) | | (SEQ ID NO: 520) | NO: 521) |
| S1-RBD- | QVQLVQSGAEV | IGWVRQMPGKG | RYSPSFQGQVTIS | WGQGTM |
| R3-T1- | KKPGESLKISCK | LEWMGI | ADKSISTAYLQW | VTVSS |
| B4 | GS | (SEQ ID NO: 523) | SSLKASDTAMYY | (SEQ ID |
| | (SEQ ID NO: 522) | | C | NO: 525) |
| | | | (SEQ ID NO: 524) | |
| S1-RBD- | QVQLVQSGAEV | IAWVRQMPGKG | RYSPSLQGQVTIS | WGQGTT |
| R3-E1- | KKPGNSLKISCK | LEWLGS | VDKSLSTAYLQW | VTVSS |
| E7 | GS | (SEQ ID NO: 527) | RSLKASDTAMYY | (SEQ ID |
| | (SEQ ID NO: 526) | | C | NO: 529) |
| | | | (SEQ ID NO: 528) | |
| S1-RBD- | EVQLVQSGAEV | IGWVRQMPGKG | RYSPSFQGQVTIS | WGQGTL |
| R3-T1- | KKPGESLKISCK | LEWMGI | ADKSISTAYLQW | VTVSS |
| C8 | GS | (SEQ ID NO: 531) | SSLKASDTAMYY | (SEQ ID |
| | (SEQ ID NO: 530) | | C | NO: 533) |
| | | | (SEQ ID NO: 532) | |
| S1-RBD- | QVQLVQSGVEV | IGWVRQMPGKG | RYSPSFQGQVTIS | WGQGTL |
| R3-T1- | KKPGESLKISCK | LEWMGI | ADKSISTAYLQW | VTVSS |
| D7 | GS | (SEQ ID NO: 535) | SSLKASDTAMYY | (SEQ ID |
| | (SEQ ID NO: 534) | | C | NO: 537) |
| | | | (SEQ ID NO: 536) | |
| RBD-R3- | QVQLQQSGPGL | WNWIRQSPSRGL | DYAVSVKSRVTI | WGQGTL |
| E1-F5 | VKPSQTLSLTCA | EWLGR | NADTSKNQLSLQ | VTVSS |
| | IF | (SEQ ID NO: 539) | LNSVTPEDTAVY | (SEQ ID |
| | (SEQ ID NO: 538) | | YC | NO: 541) |
| | | | (SEQ ID NO: 540) | |
| Ab_13 | QVTLKESGPKL | VGWIRQPPGKAL | RYSPSLKSRLTIA | WGQGTM |
| | VKPTQTLTLTCT | EWLAL | KDTSKYQVVLTM | VTVSA |
| | FS | (SEQ ID NO: 851) | TNMDPVDTATYY | (SEQ ID |
| | (SEQ ID NO: 850) | | C | NO: 853) |
| | | | (SEQ ID NO: 852) | |
| Ab_14 | QVQLQQSGPGL | WNWIRQSPARG | DYAISMKSRITINP | WGQGTL |
| | VKPSQTLSLTCA | LEWLGR | DTSKNQFSLQLES | VTVSS |
| | IS | (SEQ ID NO: 855) | VTPEDTAVYYC | (SEQ ID |
| | (SEQ ID NO: 854) | | (SEQ ID NO: 856) | NO: 857) |
| Ab_15 | QVQLVQSGGGL | MSWFRQAPGKG | GYAASVKGRFTIS | WGKGTT |
| | VKPGRSLRLSCT | LEWVGF | RDDSKSIAYLQM | VTVSS |
| | AS | (SEQ ID NO: 859) | NSLKTEDTAVYY | (SEQ ID |
| | (SEQ ID NO: 858) | | C | NO: 861) |
| | | | (SEQ ID NO: 860) | |
| Ab_16 | QVQLVQSGGGV | MHWVRQAPGK | DYADSVKGRFTV | WGQGTL |
| | VQPGRSLRLSC | GLEWVAL | SRDNSKNTLYLQ | VTVSS |
| | AAS | (SEQ ID NO: 863) | MNSLRPEDTAVY | (SEQ ID |
| | (SEQ ID NO: 862) | | FC | NO: 865) |
| | | | (SEQ ID NO: 864) | |
| Ab_17 | QVQLVQSGGGV | MHWVRQVPGK | GYADSVKGRFTIS | WGQGTT |
| | VQPGRSLRLSC | GLEWVSG | RDNVKNSLYLQM | VTVSS |
| | AAS | (SEQ ID NO: 867) | NSLRTEDSALYY | (SEQ ID |
| | (SEQ ID NO: 866) | | C | NO: 869) |
| | | | (SEQ ID NO: 868) | |
| Ab_18 | EVQLVQSGGGV | MHWVRQAPGK | YYADSVEGRFTIS | WGQGTL |
| | VQPGRSLRVSC | GLEWVAF | RDNSRNTVSLEM | VTVSS |
| | AAS | (SEQ ID NO: 871) | NSLRGEDTAVYY | (SEQ ID |
| | (SEQ ID NO: 870) | | C | NO: 873) |
| | | | (SEQ ID NO: 872) | |
| Ab_19 | EVQLVESGAEV | IGWVRQMPGKG | RYSPSFQGQVTIS | WGQGTM |
| | KKPGESLKISCK | LEWMGI | ADKSISTAYLQW | VTVSS |
| | GS | (SEQ ID NO: 875) | SSLKASDTAMYY | (SEQ ID |
| | (SEQ ID NO: 874) | | C | NO: 877) |
| | | | (SEQ ID NO: 876) | |
| Ab_20 | QVQLVQSGGGS | MHWVRQAPGK | YYVDSVKGRFTIS | WGQGTL |
| | VKPGGSLRLSC | GLEWVAN | RDNAKNSLYLQM | VTVSS |
| | AAS | (SEQ ID NO: 879) | NSLRAEDTAVYY | (SEQ ID |
| | (SEQ ID NO: 878) | | C | NO: 881) |
| | | | (SEQ ID NO: 880) | |
| Ab_21 | QVQLVQSGGGL | MHWVRQAPGK | GYVDSVKGRFTIS | WGLGTL |
| | VQPGGSLRLSC | GLEWVSG | RDNRNNKVYLQ | VTVSS |
| | ATS | (SEQ ID NO: 883) | MNNLRAEDTAVY | (SEQ ID |
| | (SEQ ID NO: 882) | | YC | NO: 885) |
| | | | (SEQ ID NO: 884) | |
| Ab_22 | QVQLVQSGGGV | MHWVRQAPGK | YYADSVKGRFTIS | WGQGTT |
| | VQPGRSLRLSC | GLEWLAV | RDKSKNTLYLQIN | VTVSS |
| | AAS | (SEQ ID NO: 887) | SLRAEDTAVYYC | (SEQ ID |
| | (SEQ ID NO: 886) | | (SEQ ID NO: 888) | NO: 889) |
| Ab_23 | EVQLVQSGAEV | ITWVRQAPGQGL | NYAQKFQGRVTI | WGQGTL |
| | KKPGSSVKVSC | EWMGR | TADTSVSTAWME | VTVSS |
| | RSS | (SEQ ID NO: 891) | LSSLESDDTAIYY | (SEQ ID |
| | (SEQ ID NO: 890) | | C | NO: 893) |
| | | | (SEQ ID NO: 892) | |
| Ab_24 | QVQLVQSGAEV | VSWVRQAPGQG | NYAQKFQGRITIT | WGQGTL |
| | KTPGSSVKVSC | LEWMGG | ADEATSTVYMAL | VTVSS |
| | KAS | (SEQ ID NO: 895) | SSLRSEDTAMYY | (SEQ ID |
| | (SEQ ID NO: 894) | | C | NO: 897) |
| | | | (SEQ ID NO: 896) | |
| Ab_25 | QVQLVQSGGGL | MSWVRQAPGKG | YYVDSVKGRFTIS | WGQGTT |
| | VQPGGSLRLSC | LEWVAN | RDNAKNSLYLQM | VTVSS |
| | AAS | (SEQ ID NO: 899) | NSLRAEDTAVYY | (SEQ ID |
| | (SEQ ID NO: 898) | | C | NO: 901) |
| | | | (SEQ ID NO: 900) | |
| Ab_26 | QVQLVQSGGGL | MHWVRQVPGK | GYADSVKGRFTIS | WGQGTM |
| | VQPGRSLRLSC | GLEWVSG | RDNAKNSLYLQM | VTVSS |
| | AAS | (SEQ ID NO: 903) | NSLRAEDTALYY | (SEQ ID |
| | (SEQ ID NO: 902) | | C | NO: 905) |
| | | | (SEQ ID NO: 904) | |
| Ab_27 | QVQLVQSGTEV | LHWVRQAPGQG | NYAQKFQGRVTL | WGQGTL |
| | KKPGASVKVSC | LEWMGW | TRDTSISTAYMEL | VTVSS |
| | KAS | (SEQ ID NO: 907) | SGLRSDDTAVYY | (SEQ ID |
| | (SEQ ID NO: 906) | | C | NO: 909) |
| | | | (SEQ ID NO: 908) | |
| Ab_28 | QVTLKESGPGL | WSWIRQSPGKGL | IYNPSLKSRLTMSI | WGQGTL |
| | VNPSETLSLTCT | EWIGY | DTSKNQLSLNLRS | VTVSS |
| | VS | (SEQ ID NO: 911) | LTAADTAVYYC | (SEQ ID |
| | (SEQ ID NO: 910) | | (SEQ ID NO: 912) | NO: 913) |
| Ab_38 | QVTLKESGPTL | VGWIRQPPGKAL | RYSPSLRSRLTITR | WGQGTL |
| | VKPTQTLTLTCT | EWLAL | DTSKNQVVLTVT | VTVSS |
| | FS | (SEQ ID NO: 990) | DMDPADTGTYYC | (SEQ ID |
| | (SEQ ID NO: 989) | | (SEQ ID NO: 991) | NO: 992) |
| wcS2- | EVQLVQSGGGV | MHWVRQAPGK | YYADSVKGRFTIS | WGQGTT |
| T4-E7 | VQPGRSLRLSC | GLEWVAV | RDNSKNTLYLQM | VTVSS |
| | AAS | (SEQ ID NO: 391) | NSLRAEDTAVYY | (SEQ ID |
| | (SEQ ID NO: | | C | NO: 377) |
| | 1341) | | (SEQ ID NO: 1385) | |
| wcS2- | QVQLV*SGGGL | MHWVRQAPGK | GYADSVKGRFTIS | WGQGTL |
| E1-A9 | VQPGRSLRLSC | GLEWVSG | RDNAKNSLYLQM | VTVSS |
| | AAS | (SEQ ID NO: 495) | DSLRAEDTAVYY | (SEQ ID |
| | (SEQ ID NO: 394) | | C | NO: 369) |
| | | | (SEQ ID NO: 1386) | |
| wcS2- | QV*LVQSGGGV | MHWVRQAPGK | YYADSVKGRFTIS | WGQGTL |
| T4-H8 | VQPGRSLRLSC | GLEWVAV | RDNSKNTLYLQM | VTVSS |
| | AAS | (SEQ ID NO: 391) | NSLRAEDTAVYY | (SEQ ID |
| | (SEQ ID NO: 378) | | C | NO: 369) |
| | | | (SEQ ID NO: 1385) | |
| wcS2- | QVQLVQSGGGV | MHWVRQAPGK | YYADSVKGRFTIS | WGQGTL |
| T3-F5 | VQPGRSLRLSC | GLEWVAV | RDNAKNSLYLQM | VTVSS |
| | AAS | (SEQ ID NO: 391) | NSLRDEDTAVYY | (SEQ ID |
| | (SEQ ID NO: 378) | | 40 | NO: 369) |
| | | | (SEQ ID NO: 1387) | |
| wcS2- | EVQLVQSGGGV | MHWVRQAPGK | YYADSVKGRFTIS | WGQGTL |
| E2-C1 | VQPGRSLRLSC | GLEWVAV | RDNSKNTLYLQM | VTVSS |
| | AAS | (SEQ ID NO: 391) | NSLRAEDTAVYY | (SEQ ID |
| | (SEQ ID NO: | | C | NO: 369) |
| | 1341) | | (SEQ ID NO: 1385) | |
| wcS2- | QVQLVQSGGGV | MHWVRQAPGK | YYADSVKGRFTIS | WGQGTL |
| T4-C9 | VQPGRSLRLSC | GLEWVAV | RDNSKNTLYLQM | VTVSS |
| | AAS | (SEQ ID NO: 391) | NSLRAEDTAVYY | (SEQ ID |
| | (SEQ ID NO: 378) | | C | NO: 369) |
| | | | (SEQ ID NO: 1385) | |
| wcS2- | QVQLVQSGGGV | MHWVRQAPGK | YYADSVKGRFTIS | WGQGTL |
| T3-B9 | VQPGRSLRLSC | GLEWVAV | RDNSKNTLYLQM | VTVSS |
| | AAS | (SEQ ID NO: 391) | NSLRAEDTAVYY | (SEQ ID |
| | (SEQ ID NO: 378) | | C | NO: 369) |
| | | | (SEQ ID NO: 1385) | |
| wcS2- | QV*LVQSGAEV | IAWVRQLPGQGL | RYSPSFQGQVTIS | WGQGTT |
| E3-C8 | KKPGESLKISCK | EWMGI | ADKSINTAYLQW | VTVSS |
| | GS | (SEQ ID NO: 1362) | SRLKASDTAKYY | (SEQ ID |
| | (SEQ ID NO: 418) | | C | NO: 377) |
| | | | (SEQ ID NO: 1388) | |
| wcS2- | QVELVQSGAEV | MHWVRQAPGQ | KYAEKFQGWVT | WGQGTM |
| T4-F8 | KEPGASVKVSC | GLEWMGW | MTRDTSISTAYM | VTVSS |
| | KDS | (SEQ ID NO: 1363) | ELSRLKSDDTALY | (SEQ ID |
| | (SEQ ID NO: | | YC | NO: 405) |
| | 1342) | | (SEQ ID NO: 1389) | |
| wcS2- | EVQLVQSGGGV | MHWVRQAPGK | YYADSVKGRFTIS | WGQGTL |
| T3-F1 | VQPGRSLRLSC | GLEWVAV | RDNSKNTLYLQM | VTVSS |
| | AAS | (SEQ ID NO: 391) | NSLRAEDTAVYY | (SEQ ID |
| | (SEQ ID NO: | | C | NO: 369) |
| | 1341) | | (SEQ ID NO: 1385) | |
| wcS2- | QVQLVQSGGGV | MHWVRQAPGK | YYADSVKGRFTIS | WGKGTL |
| E2-B1 | VQPGRSLRLSC | GLEWVAV | RDNSKNTLYLQM | VTVSS |
| | AAS | (SEQ ID NO: 391) | NSLRAEDTAVYY | (SEQ ID |
| | (SEQ ID NO: 378) | | C | NO: 429) |
| | | | (SEQ ID NO: 1385) | |
| wcS2- | QVQLQESGGGV | MHWVRQAPGK | YYADSVKGRFTIS | WGQGTL |
| T2-G3 | VQPGRSLRLSC | GLEWVAV | RDNSKNTLYLQM | VTVSS |
| | AAS | (SEQ ID NO: 391) | NSLRAEDTAVYY | (SEQ ID |
| | (SEQ ID NO: | | C | NO: 369) |
| | 1343) | | (SEQ ID NO: 1385) | |
| wcS2- | QV*LVQSGAEV | IAWVRQLPGQGL | RYSPSFQGQVTIS | WGQGTT |
| T1-A6 | KKPGESLKISCK | EWMGI | ADKSINTAYLQW | VTVSS |
| | GS | (SEQ ID NO: 1362) | SRLKASDTAKYY | (SEQ ID |
| | (SEQ ID NO: 418) | | C | NO: 377) |
| | | | (SEQ ID NO: 1388) | |
| wcS2- | EV*LVQSGGGV | MHWVRQAPGK | YYADSVKGRFTIS | WGQGTT |
| T4-D4 | VQPGRSLRLSC | GLEWVAV | RDNSKNTLYLQM | ATASS |
| | AAS | (SEQ ID NO: 391) | NSLRAEDTAVYY | (SEQ ID |
| | (SEQ ID NO: | | C | NO: 1416) |
| | 1341) | | (SEQ ID NO: 1385) | |
| wcS2- | EVQLVQSGGGV | MHWVRQAPGK | YYADSVKGRFTIS | WGQGTL |
| T2-D10 | VQPGRSLRLSC | GLEWVAV | RDNSKNTLYLQM | VTVSS |
| | AAS | (SEQ ID NO: 391) | NSLRAEDTAVYY | (SEQ ID |
| | (SEQ ID NO: | | C | NO: 369) |
| | 1341) | | (SEQ ID NO: 1385) | |
| wcS2- | QVQLVQSGGGV | MHWVRQAPGK | YYADSVKGRFTIS | WGQGTL |
| T1-G9 | VQPGRSLRLSC | GLEWVAV | RDNSKNTLYLQM | VTVSS |
| | AAS | (SEQ ID NO: 391) | NSLRAEDTAVYY | (SEQ ID |
| | (SEQ ID NO: 378) | | C | NO: 369) |
| | | | (SEQ ID NO: 1385) | |
| wcS2- | QVQLVQSGGGV | MHWVRQAPGK | YYADSVKGRFTIS | WGQGTL |
| E3-H7 | VQPGRSLRLSC | GLEWVAG | RDNSKNTLYLQM | VTVSS |
| | AAS | (SEQ ID NO: 1364) | NSLRAEDTAVYY | (SEQ ID |
| | (SEQ ID NO: 378) | | C | NO: 369) |
| | | | (SEQ ID NO: 1385) | |
| wcS2- | QVTLKESGPTL | VGWIRQPPGKAL | RYSPSLKSRLTISK | WGQGTM |
| T2-C11 | VKPTQTLTLTCT | EWLAL | DTSRNQVVLTMT | VTVSS |
| | FS | (SEQ ID NO: 387) | NMDPADTGTYYC | (SEQ ID |
| | (SEQ ID NO: 466) | | (SEQ ID NO: 1390) | NO: 405) |
| SARS2- | QVQLVQSGGGL | MHWVRQAPGK | YYADSVQGRITIS | WGQGTL |
| R3-G2- | VKPGGSLRLSCS | GLEWVAV (SEQ | RDNSKNTLYLQM | VTVSS |
| P1- | AS (SEQ ID NO: | ID NO: 391) | NSLRAEDTAVYF | (SEQ ID |
| H9_PelB- | 1345) | | C (SEQ ID NO: | NO: 369) |
| F_2020 | | | 1391) | |
| Jun. | | | | |
| 19_D08 | | | | |
| SARS2- | QVQLVQSGGGV | MHWVRQAPGK | YYADSVKGRFTIS | WGQGTL |
| R3-G3- | VQPGRSLRLSC | GLEWVAV (SEQ | RDNSKNTLYLQM | VTVSS |
| P1- | AAS (SEQ ID | ID NO: 391) | NSLRAEDTAVYY | (SEQ ID |
| F8_PelB- | NO: 378) | | C (SEQ ID NO: | NO: 369) |
| F_2020 | | | 1385) | |
| Jun. | | | | |
| 19_F11 | | | | |
| SARS2- | QVQLVQSGGGV | MHWVRQAPGK | YYADSVKGRFTIS | WGQGTL |
| R3-G2- | VQPGRSLRLSC | GLEWVAV (SEQ | RDNSKNTLYLQM | VTVSS |
| P1- | AAS (SEQ ID | ID NO: 391) | NSLRTEDTAVYY | (SEQ ID |
| B4_PelB- | NO: 378) | | C (SEQ ID NO: | NO: 369) |
| F_2020 | | | 1392) | |
| Jun. | | | | |
| 19_B02 | | | | |
| SARS2- | QVQLVQSGGGV | MHWVRQAPGK | YYADSVKGRFTIS | WGQGTT |
| R3-G3- | VQPGRSLRLSC | GLEWVAV (SEQ | RDNSKNTLYLQM | VTVSS |
| P1- | AAS (SEQ ID | ID NO: 391) | NSLRAEDTAVYY | (SEQ ID |
| B2_PelB- | NO: 378) | | C (SEQ ID NO: | NO: 377) |
| F_2020 | | | 1385) | |
| Jun. | | | | |
| 19_A08 | | | | |
| SARS2- | QVQLQESGGGV | MHWVRQAPGK | YYADSVKGRFTIS | WGQGTM |
| R3-G3- | VQPGRALRLSC | GLEWVAV (SEQ | RDNSKNTLYLQM | VTVSS |
| P1- | RAS (SEQ ID | ID NO: 391) | NSLRAEDTAVYY | (SEQ ID |
| B3_PelB- | NO: 1346) | | C (SEQ ID NO: | NO: 405) |
| F_2020 | | | 1385) | |
| Jun. | | | | |
| 19_B08 | | | | |
| SARS2- | *VQLVQSGAEV | ISWVRQAPGQG | NYAQKFQGRVTI | WGQGTM |
| R3-G1- | KKPGSSVKVSC | LEWMGG (SEQ | TADKSTSTAYME | VTVSS |
| P3- | KAS (SEQ ID | ID NO: 435) | LSSLRSEDTAVYY | (SEQ ID |
| E8_PelB- | NO: 430) | | C (SEQ ID NO: | NO: 405) |
| F_2020 | | | 1393) | |
| Jun. | | | | |
| 19_H04 | | | | |
| SARS2- | EVQLVQSGAEV | ISWVRQAPGQG | NYAQKLQGRVT | WGQGTT |
| R3-G1- | KKPGASVKVSC | LEWMGW (SEQ | MTTNTSTNTAYM | VTVSS |
| P2- | KAS (SEQ ID | ID NO: 427) | ELRSLRSDDTAG | (SEQ ID |
| D7_PelB- | NO: 454) | | YYW (SEQ ID NO: | NO: 377) |
| F_2020 | | | 1394) | |
| Jun. | | | | |
| 19_H09 | | | | |
| SARS2- | QVTLKESGPTL | VGWIRQPPGKA | RYSPSLKSRLTIT | WGQGTL |
| R3-G1- | VKPTQTLTLTCT | LEWLAL (SEQ | KDTSKNQVVLTM | VTVSS |
| P1- | FS (SEQ ID NO: | ID NO: 387) | TNMDPVDTATYY | (SEQ ID |
| C6_PelB- | 466) | | C (SEQ ID NO: | NO: 369) |
| F_2020 | | | 388) | |
| Jun. | | | | |
| 19_H02 | | | | |
| SARS2- | QVQLVQSGGGL | MHWVRQAPGK | YYAYSVQGRFTIS | RGRGTPG |
| R3-T-P2- | VQPGGSLRLSCS | GLEWVEV (SEQ | KNNSKNTLYLQM | TASS |
| E12 PelB- | AS (SEQ ID NO: | ID NO: 1365) | NSLKAEDTAVYF | (SEQ ID |
| F_2020 | 1347) | | C (SEQ ID NO: | NO: 1417) |
| Jun. | | | 1395) | |
| 19_D07 | | | | |
| SARS2- | QVQLVQSGGGV | MHWVRQAPGK | YYADSVKGRFTIS | WGQGTM |
| R3-G2- | VQPGRSLRLSC | GLEWVAV (SEQ | RDNSKNTLYLQM | VTVSS |
| P1- | AAS (SEQ ID | ID NO: 391) | NSLRVEDTAVYY | (SEQ ID |
| D12 PelB- | NO: 378) | | C (SEQ ID NO: | NO: 405) |
| F_2020 | | | 1396) | |
| Jun. | | | | |
| 19_H04 | | | | |
| SARS2- | EVQLVQSGGGV | MHWVRQAPGK | YYADSVKGRFTIS | WGQGTL |
| R3-G1- | VQPGRSLRLSC | GLEWVAV (SEQ | RDNSKNTLYLQM | VTVSS |
| P2- | AAS (SEQ ID | ID NO: 391) | NSLRAEDTAVYY | (SEQ ID |
| E8_PelB- | NO: 1341) | | C (SEQ ID NO: | NO: 369) |
| F_2020 | | | 1385) | |
| Jun. | | | | |
| 19_E10 | | | | |
| SARS2- | EVQLVQSGGGV | MHWVRQAPGK | YYADSVKGRFTIS | WGQGTT |
| R3-G1- | VQPGRSLTLSCA | GLEWVAV (SEQ | RDNSKNTLYLQM | VTVSS |
| P1- | AS (SEQ ID NO: | ID NO: 391) | NSLRAEDTAVYY | (SEQ ID |
| A10 PelB- | 1348) | | C (SEQ ID NO: | NO: 377) |
| F_2020 | | | 1385) | |
| Jun. | | | | |
| 19_H01 | | | | |
| SARS2- | QVQLVQSGGGV | MHWVRQAPGK | YYADSVKGRFTIS | WGQGTL |
| R3-G1- | VQPGRSLRLSC | GLEWVAL (SEQ | RDNAKNSLYLQM | VTVSS |
| P3- | AAS (SEQ ID | ID NO: 403) | NSLRAEDTAVYY | (SEQ ID |
| B5_PelB- | NO: 378) | | C (SEQ ID NO: | NO: 369) |
| F_2020 | | | 1397) | |
| Jun. | | | | |
| 19_D02 | | | | |
| SARS2- | QVQLVQSGTEV | ISWVRQAPGQG | NYAQKFQGRVTI | WGQGTT |
| R3-G3- | RQPGASVKVSC | LEWMGG (SEQ | TADESTSTAYME | VTVSS |
| P1- | KAS (SEQ ID | ID NO: 435) | LSSLRSEDTAVYY | (SEQ ID |
| A2_PelB- | NO: 1349) | | C (SEQ ID NO: | NO: 377) |
| F_2020 | | | 1398) | |
| Jun. | | | | |
| 19_B07 | | | | |
| SARS2- | EVQLVQSGGGV | MHWVRQAPGK | YYADSVKGRFTIS | WGKGTL |
| R3-G3- | VQPGRSLRLSC | GLEWVAV (SEQ | RDNSKNTLYLQM | VTVSS |
| P1- | AAS (SEQ ID | ID NO: 391) | NSLRAEDTAVYY | (SEQ ID |
| D1_PelB- | NO: 1341) | | C (SEQ ID NO: | NO: 429) |
| F_2020 | | | 1385) | |
| Jun. | | | | |
| 19_G09 | | | | |
| SARS2- | QVQLVQSGGGV | LHWVRQAPGKG | YYADSVKGRFTIS | WGQGTM |
| R3-G1- | VRPGRSLRLSCA | LEWVAV (SEQ | RDNAKNSLYLQM | VTVSS |
| P2- | AS (SEQ ID NO: | ID NO: 1366) | NSLRAEDTAVYY | (SEQ ID |
| D4_PelB- | 1350) | | C (SEQ ID NO: | NO: 405) |
| F_2020 | | | 1397) | |
| Jun. | | | | |
| 19_G09 | | | | |
| SARS2- | EVQLVQSGGGV | MHWVRQAPGK | YYADSVKGRFTIS | WGQGTL |
| R3-G1- | VQPGRSLRLSC | GLEWVAV (SEQ | RDNSKNTLYLQM | VTVSS |
| P1- | AAS (SEQ ID | ID NO: 391) | NSLRAEDTAIYYC | (SEQ ID |
| B6_PelB- | NO: 1341) | | (SEQ ID NO: 1399) | NO: 369) |
| F_2020 | | | | |
| Jun. | | | | |
| 19_E02 | | | | |
| SARS2- | QVTLKESGPAL | VGWIRQPPGKAL | RYSPSLKSRLTIT | WGQGTL |
| R3-G3- | VKPTQTLTLTCT | EWLAL (SEQ ID | KDTSKNQVVLTM | VTVSS |
| P1- | FS (SEQ ID NO: | NO: 387) | TNMDPVDTATYY | (SEQ ID |
| G8_PelB- | 1351) | | C (SEQ ID NO: | NO: 369) |
| F_2020 | | | 388) | |
| Jun. | | | | |
| 25_A05 | | | | |
| SARS2- | QVTLKESGPTL | VGWIRQPPGKAL | RYSPSLKSRLTIT | WGQGTT |
| R3-G1- | VKPTQTLTLTCT | EWLAL (SEQ ID | KDTSKNQVVLTM | VTVSS |
| P1- | LS (SEQ ID NO: | NO: 387) | TNMDPVDTATYY | (SEQ ID |
| F11_PelB- | 410) | | C (SEQ ID NO: | NO: 377) |
| F_2020 | | | 388) | |
| Jun. | | | | |
| 25_B05 | | | | |
| SARS2- | QVQLVQSGGGV | MHWVRQAPGK | HYADSVKGRFTIS | WGQGTL |
| R3-T1- | VQPGRSRRLSCT | GLEWVST (SEQ | RDNSKNTLYLQM | VTVSS |
| P3- | AS (SEQ ID NO: | ID NO: 1367) | KSLRAEDTATYY | (SEQ ID |
| F4_PelB- | 1352) | | C (SEQ ID NO: | NO: 369) |
| F_2020 | | | 1400) | |
| Jun. | | | | |
| 20_C03 | | | | |
| SARS2- | QVQLVQSGGGL | MSWVRQAPGKG | YYADSVKGRFTIS | WGQGTL |
| R3-G1- | VQPGGSLRLSC | LEWVSA (SEQ | RDNSKNTLYLQM | VTVSS |
| P2- | AAS (SEQ ID | ID NO: 1368) | NSLRAEDTGVYY | (SEQ ID |
| B8_PelB- | NO: 438) | | C (SEQ ID NO: | NO: 369) |
| F_2020 | | | 1401) | |
| Jun. | | | | |
| 25_C06 | | | | |
| SARS2- | QVQLVQSGGGV | MHWVRQAPGK | YYADSVKGRFTIS | WGQGTL |
| R3-G1- | VQPGRSLRLSC | GLEWVAV (SEQ | RDNSKNTLYLQM | VTVSS |
| P2- | AAS (SEQ ID | ID NO: 391) | NSLRAEDTAVYY | (SEQ ID |
| D3_PelB- | NO: 378) | | C (SEQ ID NO: | NO: 369) |
| F_2020 | | | 1385) | |
| Jun. | | | | |
| 25_C12 | | | | |
| SARS2- | EVQLVQSGGGV | MHWVRQAPGK | YYADSVKGRFTIS | WGQGTL |
| R3-G1- | VQPGRSLRLSC | GLEWVSY (SEQ | RDNAKNSLYLQM | VTVSS |
| P4- | AAS (SEQ ID | ID NO: 1369) | NSLRAEDTAVYY | (SEQ ID |
| C5_PelB- | NO: 1341) | | C (SEQ ID NO: | NO: 369) |
| F_2020 | | | 1397) | |
| Jun. | | | | |
| 25_C07 | | | | |
| SARS2- | QVQLVQSGAEV | FSWVRQAPGQG | DYAQNFQGRVT | WGQGTL |
| R3-G1- | KKPGASVKVSC | LEWMGW (SEQ | MTTDTSTNTAYM | VTVSS |
| P2- | KAS (SEQ ID | ID NO: 1370) | ELRSLRSDDTAV | (SEQ ID |
| D6_PelB- | NO: 462) | | YYC (SEQ ID NO: | NO: 369) |
| F_2020 | | | 1402) | |
| Jun. | | | | |
| 25_D02 | | | | |
| SARS2- | EVQLVQSGAEV | MHWVRQAPGQ | SYAQKFQGRVTM | WGQGTT |
| R3-G1- | KKPGASVKVSC | GLEWMGI (SEQ | TRDTSTSTVYME | VTVSS |
| P1- | KAS (SEQ ID | ID NO: 1371) | LSSLRSEDTAVYY | (SEQ ID |
| B7_PelB- | NO: 454) | | C (SEQ ID NO: | NO: 377) |
| F_2020 | | | 1403) | |
| Jun. | | | | |
| 25_A03 | | | | |
| 95_PelB- | QVQLVQSGGGV | MHWVRQAPGK | YYADSVKGRFTIS | WGQGTT |
| F_2020 | VQPGRSLRLSC | GLEWVAV (SEQ | RDNSKKMLYLQ | VTVSS |
| Jun. | AAS (SEQ ID | ID NO: 391) | MNSLTAEDTAVY | (SEQ ID |
| 25_G11 | NO: 378) | | YC (SEQ ID NO: | NO: 377) |
| | | | 1404) | |
| 73_PelB- | QVQLVQSGGGL | MNWVRQAPGK | YYADSVKGRFTIS | WGQGTL |
| F_2020 | VQPGGSLRLSC | GLEWVAV (SEQ | RDNSKNTLYLQM | VTVSS |
| Jun. | AAS (SEQ ID | ID NO: 1372) | NSLRAEDTAVYY | (SEQ ID |
| 25_F09 | NO: 438) | | C (SEQ ID NO: | NO: 369) |
| | | | 1385) | |
| SARS2- | QVQLVQSGGGL | MHWVRQAPGK | YYADSVKGRFTIS | WGQGTL |
| R3-G3- | VKPGGSLRLSC | GLEWVAV (SEQ | RDNSKNTLYLQM | VTVSS |
| P1- | AAS (SEQ ID | ID NO: 391) | NSLRAEDTAVYY | (SEQ ID |
| G9_PelB- | NO: 474) | | C (SEQ ID NO: | NO: 369) |
| F_2020 | | | 1385) | |
| Jun. | | | | |
| 25_B05 | | | | |
| SARS2- | QVQLVQSGAEV | MHWVRQAPGQ | NYAQKFQGRVT | WGQGTT |
| R3-G1- | KKPGASVKVSC | GLEWMGR (SEQ | MTRDTSISTAYM | VTVSS |
| P3- | KAS (SEQ ID | ID NO: 1373) | ELSRLRSDDTAV | (SEQ ID |
| G7_PelB- | NO: 462) | | YYC (SEQ ID NO: | NO: 377) |
| F_2020 | | | 432) | |
| Jun. | | | | |
| 25_H01 | | | | |
| SARS2- | QVQLQQSGAEV | VSWVRQAPGQG | NYAQRFQGRVTI | WGKGTT |
| R3-G1- | KKPGSSVKVSC | PEWMGR (SEQ | TADESTNTAYME | VTVSS |
| P4- | KAS (SEQ ID | ID NO: 1374) | LSSLRSEDTAVYY | (SEQ ID |
| A2_PelB- | NO: 1353) | | C (SEQ ID NO: | NO: 861) |
| F_2020 | | | 1405) | |
| Jun. | | | | |
| 25_A06 | | | | |
| SARS2- | EVQLVQSGAEV | ISWVRQAPGQGL | NYAQKLQGRVT | WGQGTT |
| R3-G3- | KKPGASVKVSC | EWMGW (SEQ ID | MTTDTSTSTAYM | VTVSS |
| P1- | KAS (SEQ ID | NO: 427) | ELRSLRSDDTAV | (SEQ ID |
| G1_PelB- | NO: 454) | | YYC (SEQ ID NO: | NO: 377) |
| F_2020 | | | 428) | |
| Jun. | | | | |
| 25_F04 | | | | |
| SARS2- | QVQLVQSGGGV | MHWVRQAPGK | YYADSVKGRFTIS | WGQGTL |
| R3-G3- | VQPGRSLRLSC | GLEWVAV (SEQ | RDNSKNTLYLQM | VTVSS |
| P1- | AAS (SEQ ID | ID NO: 391) | NSLRDEDTAVYY | (SEQ ID |
| F1_PelB- | NO: 378) | | C (SEQ ID NO: | NO: 369) |
| F_2020 | | | 1406) | |
| Jun. | | | | |
| 25_C04 | | | | |
| SARS2- | QVQLQESGAGL | WSWIRQPPGKGL | NYNPSFQSRVAM | WGQGTL |
| R3-G1- | LRPSETLSLTCA | EWIGE (SEQ ID | SRDTPKNQFSLKL | VTVSS |
| P3- | VY (SEQ ID NO: | NO: 1375) | SSVTAADTAVYY | (SEQ ID |
| H10 PelB- | 1354) | | C (SEQ ID NO: | NO: 369) |
| F_2020 | | | 1407) | |
| Jun. | | | | |
| 25_H10 | | | | |
| 85_PelB- | QVQLV*SGAEV | VGWVRQKPGKG | RKNPSFEGQVTM | WGQGTL |
| F_2020 | KKPGESLKISCQ | LEWMGI (SEQ ID | SVDKSLHSVYLH | GTVSS |
| Jun. | GS (SEQ ID NO: | NO: 1376) | WTSLKVSDTAKY | (SEQ ID |
| 25_G10 | 1355) | | YC (SEQ ID NO: | NO: 1418) |
| | | | 1408) | |
| SARS2- | QVQLVQSGAEV | INWVRQATGQG | GYAQSFQGRVTF | WGQGTT |
| R3-G1- | KKPGASVKVSC | LEWMGW (SEQ | TRDTSINTAYMEL | VTVSS |
| P4- | KAS (SEQ ID | ID NO: 1377) | SSLRSEDTAVYYC | (SEQ ID |
| A3_PelB- | NO: 462) | | (SEQ ID NO: 1409) | NO: 377) |
| F_2020 | | | | |
| Jun. | | | | |
| 25_B06 | | | | |
| SARS2- | QVQLVQSGAEV | ISWVRQMPGKG | NYSPSFQGHVTIT | WGQGTM |
| R3-G1- | KKSGESLEISCK | LEWMGR (SEQ | VDKSTGAAYLH | VTVSS |
| P3- | GS (SEQ ID NO: | ID NO: 1378) | WSSLKASDTGMY | (SEQ ID |
| H6_PelB- | 1356) | | YC (SEQ ID NO: | NO: 405) |
| F_2020 | | | 1410) | |
| Jun. | | | | |
| 25_H08 | | | | |
| SARS2- | EVQLVQSGAEV | IGWVRQMPGKG | IYSPSFQGQVTISA | WGQGTM |
| R3-G3- | KKPGESLRISCK | LEWMGV (SEQ | DKSISTAYLQWSS | VTVSS |
| P1- | GS (SEQ ID NO: | ID NO: 1379) | LKASDTAIYYC | (SEQ ID |
| G5_PelB- | 1357) | | (SEQ ID NO: 1411) | NO: 405) |
| F_2020 | | | | |
| Jun. | | | | |
| 25_H04 | | | | |
| SARS2- | EVQLVESGGGQ | MHWVRQAPGK | YYADSLKGRFNIS | WGHGTL |
| R3-G1- | VKPGGSLRISCA | GLEWVSS (SEQ | RDNAKKLLYLQL | VTVSS |
| P4- | AS (SEQ ID NO: | ID NO: 1380) | SSLSAEDTALYFC | (SEQ ID |
| C10 PelB- | 1358) | | (SEQ ID NO: 1412) | NO: 481) |
| F_2020 | | | | |
| Jun. | | | | |
| 25_F07 | | | | |
| SARS2- | QVQLVQSGAEV | ISWVRQAPGQGL | NYAQKLQGRVT | WGQGTL |
| R3-G1- | KKPGSSVKVSC | ELMGW (SEQ ID | MTTDTSTSTAYM | VTVSS |
| P1- | KAS (SEQ ID | NO: 1381) | ELRSLRSGDTAV | (SEQ ID |
| F10 PelB- | NO: 430) | | YYC (SEQ ID NO: | NO: 369) |
| F_2020 | | | 1413) | |
| Jun. | | | | |
| 25_B04 | | | | |
| SARS2- | EVQLVESGPGL | WSWIRQHPGKG | YYNPSLKSRVTIS | WGQGTM |
| R3-T1- | VKPSQTLSLTCT | LEWIGY (SEQ ID | VDTSKNQFSLKLS | VTVSS |
| P4- | VS (SEQ ID NO: | NO: 1382) | SVTAADTAVYYC | (SEQ ID |
| H4_PelB- | 1359) | | (SEQ ID NO: 1414) | NO: 405) |
| F_2020 | | | | |
| Jun. | | | | |
| 20_H04 | | | | |
| SARS2- | QVQLVQSGGGV | MHWVRQAPGK | YYADSVKGRFTIS | WGQGTL |
| R3-G2- | VRPGRSLRLSCA | GLEWVAV (SEQ | RDNAKNSLYLQM | VTVSS |
| P1- | AS (SEQ ID NO: | ID NO: 391) | NSLRAEDTAVYY | (SEQ ID |
| E8_PelB- | 1350) | | C (SEQ ID NO: | NO: 369) |
| F_2020 | | | 1397) | |
| Jun. | | | | |
| 25_A02 | | | | |
| SARS2- | QVQLVQSGGGL | MHWVRQAPGK | YYADSVKGRFTIS | WGQGTL |
| R3-G1- | VQPGGSLRLSC | GLEWVAV (SEQ | RDNSKNTLYLQM | VTVSS |
| P1- | AAS (SEQ ID | ID NO: 391) | NSLRAEDTAVYY | (SEQ ID |
| C3_PelB- | NO: 438) | | C (SEQ ID NO: | NO: 369) |
| F_2020 | | | 1385) | |
| Jun. | | | | |
| 25_A07 | | | | |
| SARS2- | EVQLVQSGGGL | MHWVRQAPGK | DYAESVKGRFTIS | WGQGTL |
| R3-G3- | VQPGGSLRLPCS | GLEHISI (SEQ ID | RDNSKNTLYLQM | VTVSS |
| P1- | AS (SEQ ID NO: | NO: 1383) | TSLREEDTAVYY | (SEQ ID |
| G2_PelB- | 1360) | | C (SEQ ID NO: | NO: 369) |
| F_2020 | | | 1415) | |
| Jun. | | | | |
| 25_G04 | | | | |
| SARS2- | QVQLVQSGAEV | ISWVRQAPGQGP | NYAQRFQGRVTI | WGKGTL |
| R3-G1- | KKPGSSAKVSC | EWMGR (SEQ ID | TADESTNTAYME | VTVSS |
| P4- | KAS (SEQ ID | NO: 1384) | LSSLRSEDTAVYY | (SEQ ID |
| D1_PelB- | NO: 1361) | | C (SEQ ID NO: | NO: 429) |
| F_2020 | | | 1405) | |
| Jun. | | | | |
| 25_A08 | | | | |

TABLE 64B
Light chain (V_{L}) framework regions (FRs) of the COVID-19_antibodies.

| Seq ID | FR1-IMGT | FR2-IMGT | FR3-IMGT | FR4-IMGT |
|---|---|---|---|---|
| S1-R3- | LPVLTQPPS.AS | VNWYQQLPGTA | QRPSGVPDRFSG | FGSGTKVT |
| T1-H7 | GTPGQRVTISCS | PKLLIY | SKSGTSASLAISG | VL |
| (Ab12) | GS | (SEQ ID NO: 543) | LQSEDEADYYC | (SEQ ID |
| | (SEQ ID NO: 542) | | (SEQ ID NO: 544) | NO: 545) |
| RBD-R3- | NFMLTQPHS.VS | VQWYQQRPGSS | QRPSGVPDRFSG | FGGGTKLT |
| E1-G7 | ESPGKTVTISCT | PTTVIY | SIDSSSNSASLTIS | VL |
| (AB2-10) | RS | (SEQ ID NO: 547) | GLTTEDEADYYC | (SEQ ID |
| | (SEQ ID NO: 546) | | (SEQ ID NO: 548) | NO: 549) |
| S1-RBD- | QPGLTQPPS.AS | VTWYQQLPGTA | QRPSGVPDRFSA | FGGGTKLT |
| R3-E1- | GTPGQRVTISCS | PKLLIY | SRSGTSASLAITG | VL |
| D8 | GS | (SEQ ID NO: 551) | LQAEDEADYYC | SEQ ID |
| (Ab_3) | (SEQ ID NO: 550) | | (SEQ ID NO: 552) | NO: 553) |
| S1-RBD- | NFMLTQPHS.VS | VQWYQQRPGSS | LRPSGVPDRFSG | FGGGTKLT |
| R3-T1- | ESPGNTVTISCT | PTTVIF | SIDSSSNSASLTIS | VL |
| C7 | RT | (SEQ ID NO: 555) | GLKTEDEADYY | (SEQ ID |
| | (SEQ ID NO: 554) | | C | NO: 557) |
| | | | (SEQ ID NO: 556) | |
| RBD-R3- | NFMLTQPHS.VS | VQWYRQRPGSA | QRPAGVPDRFSG | FGTGTKLT |
| T1-F4 | ESPGKTVIISCTR | PTTVIY | SVDSSSNSASLTI | VL |
| (Ab2-2) | T | (SEQ ID NO: 559) | TGLKTEDEADY | (SEQ ID |
| | (SEQ ID NO: 558) | | YC | NO: 561) |
| | | | (SEQ ID NO: 560) | |
| RBD-R3- | SYELTQPPS.VSE | VSWYQHLPGKA | RLPSGVSDRFSG | FGGGTKLT |
| E1-A5 | APRQRVTISCSG | PKLLIY | SKSGTSASLAISG | VL |
| | S | (SEQ ID NO: 563) | LRSEDEADYYC | (SEQ ID |
| | (SEQ ID NO: 562) | | (SEQ ID NO: 564) | NO: 565) |
| S1-RBD- | NFMLTQPHS.VS | VQWYQQRPGSS | QRPSGVPDRFSG | FGGGTKLT |
| R3-T1- | ESPGKTVTISCT | PTTVIY | SIDSSSNSASLTIS | VL |
| F5 | RS | (SEQ ID NO: 567) | GLKAEDEADYY | (SEQ ID |
| (Ab2-7) | (SEQ ID NO: 566) | | C | NO: 569) |
| | | | (SEQ ID NO: 568) | |
| S1-R3- | QPVLTQPPSASG | VNWYQQLPGTA | QRPSGVPDRFSD | FGTGTKVT |
| T1-A12 | TPGQRVTISCSG | PKLLIY | SKSGTSASLAISG | VL |
| (Ab_5) | S | (SEQ ID NO: 571) | LQSEDEADYFC | (SEQ ID |
| | (SEQ ID NO: 570) | | (SEQ ID NO: 572) | NO: 573) |
| S1-R3- | QSVLTQPPSASG | VSWYQQYPGKA | KRPSGVPDRFSG | FGGGTKLT |
| T1-A6 | SPGQSVTISCTG | PKLMIY | SKSGNTASLTVS | VL |
| (Ab_4) | T | (SEQ ID NO: 575) | GLRAEDEADYY | (SEQ ID |
| | (SEQ ID NO: 574) | | C | NO: 577) |
| | | | (SEQ ID NO: 576) | |
| S1-RBD- | QPGLTQPPSVSV | VHWYQQKAGQ | DRPSEIPERFSGS | FGTGTKVT |
| R3-T1- | APGKTARITCG | APVLVVY | NSGNTATLTISR | VL |
| A5 | GN | (SEQ ID NO: 579) | AEVGDEADYYC | (SEQ ID |
| | (SEQ ID NO: 578) | | (SEQ ID NO: 580) | NO: 581) |
| S1-RBD- | SSELTQDPAVSV | AGWYQQKPGQA | KRPSGIPDRFSAS | FGGGTKLT |
| R3-T1- | VLGQAVRITCQ | PVLVTY | TSGNTASLTITG | VL |
| B3 | GD | (SEQ ID NO: 583) | AQADDEADYYC | (SEQ ID |
| (Ab_1) | (SEQ ID NO: 582) | | (SEQ ID NO: 584) | NO: 585) |
| S1-RBD- | QSALTQPPSVSG | VSWYQQPPGKA | NRPSGVSNRFSG | FGTGTKVT |
| R3-T1- | SPGQSVTISCTG | PKLMIY | SKSGNTASLTISG | VL |
| E7 | T | (SEQ ID NO: 587) | LQAEDEADYYC | (SEQ ID |
| | (SEQ ID NO: 586) | | (SEQ ID NO: 588) | NO: 589) |
| S1-RBD- | QSVVTQPPSVSG | VQWYQQLPGTA | NRPSGVPDRFSA | FGGGTKVT |
| R3-T1- | APGQRVTISCTG | PKLLIW | SKSGTSASLAITG | VL |
| F9 | S | (SEQ ID NO: 591) | LQAEDEADYFC | (SEQ ID |
| | (SEQ ID NO: 590) | | (SEQ ID NO: 592) | NO: 593) |
| S1-R3- | NFMLTQPHSVS | VQWYQQRPGSS | DRPSGVPDRFSG | FGGGTQLT |
| T1-C2 | ESPGKTVTISCT | PTTVIY | SIDSSSNSASLTIS | VL |
| (Ab_6) | RS | (SEQ ID NO: 595) | GLKPEDEADYY | (SEQ ID |
| | (SEQ ID NO: 594) | | C | NO: 597) |
| | | | (SEQ ID NO: 596) | |
| RBD-R3- | QTVVTQEPAVS | ASWYQQKPGQA | KRPSGIPDRFSGS | FGGGTKLT |
| E1-B3 | VALGQTVRITC | PVLVVY | TSGNTASLTITG | VL |
| | QGD | (SEQ ID NO: 599) | AQAEDEADYYC | (SEQ ID |
| | (SEQ ID NO: 598) | | (SEQ ID NO: 600) | NO: 601) |
| RBD-R3- | EIVLTQSPATLS | LAWYQQRPGQA | TRATAIPARFSGS | FGGGTKVE |
| T1-B5 | LSPGERATLSCR | PRLLIY | GSGTEFTLTISRL | IN |
| | AS | (SEQ ID NO: 603) | EPEDFATYYC | (SEQ ID |
| | (SEQ ID NO: 602) | | (SEQ ID NO: 604) | NO: 605) |
| RBD-R3- | QSALTQPPSASG | VSWYQQHPGKA | KRPPGVPDRFSG | FGGGTKLT |
| T1-H3 | SPGQSVTISCTG | PKLLIY | SKSGNTASLTVS | VL |
| (Ab_2) | T | (SEQ ID NO: 607) | GLQAEDEADYY | (SEQ ID |
| | (SEQ ID NO: 606) | | C | NO: 609) |
| | | | (SEQ ID NO: 608) | |
| S1-R3- | QPGLTQPPSVSK | AAWLQQHQGHP | NRPPGISERFSAS | FGGGTKLS |
| T1-C4 | GLRQTATLTCT | PKLLSY | RSGNTASLTITGL | VL |
| (Ab_8) | GN | (SEQ ID NO: 611) | QPEDEADYYC | (SEQ ID |
| | (SEQ ID NO: 610) | | (SEQ ID NO: 612) | NO: 613) |
| S1-RBD- | NFMLTQPHSVS | VQWYQQRPGSA | QRPSGVPDRFSG | FGGGTKLT |
| R3-T1- | ESPGKTVTISCT | PTTVIY | SIDSSSNSASLTIS | VL |
| B12 | GS | (SEQ ID NO: 615) | GLKTEDEADYY | (SEQ ID |
| | (SEQ ID NO: 614) | | C | NO: 617) |
| | | | (SEQ ID NO: 616) | |
| S1-RBD- | NFMLTQPHSVS | VQWYQQRPGSA | QRPSGVPDRFSG | FGGGTKLT |
| R3-T1- | ESPGKTVTISCT | PTTVIY | SIGSSSNSASLTIS | VL |
| G5 | GS | (SEQ ID NO: 619) | GLKTEDEADYY | (SEQ ID |
| | (SEQ ID NO: 618) | | C | NO: 621) |
| | | | (SEQ ID NO: 620) | |
| S1-RBD- | QPGLTQPPSVSV | VHWYQQKPGRA | GRPSGIPERFSGS | FATGTKVS |
| R3-T1- | APGQTARISCGG | PVLVVY | NSGNTATLTVSR | IL |
| E2 (Ab7) | N | (SEQ ID NO: 623) | VEAGDEADYYC | (SEQ ID |
| | (SEQ ID NO: 622) | | (SEQ ID NO: 624) | NO: 625) |
| RBD-R3- | QPGLTQPASVS | VSWYQQHPGKA | KRPSGISNRFSGS | FGGGTKLT |
| T1-F7 | GSPGQSVTISCT | PKLMIY | KSGNTASLTISGL | VL |
| | GT | (SEQ ID NO: 627) | QAEDEADYFC | (SEQ ID |
| | (SEQ ID NO: 626) | | (SEQ ID NO: 628) | NO: 629) |
| S1-RBD- | QPGLTQPPSASG | VNWYQQLPGTA | QRPSGVPDRFSG | FGGGTKLT |
| R3-T1- | TPGQRVTISCSG | PKLLIY | SKSGTSASLAISG | VL |
| G1 | S | (SEQ ID NO: 631) | LRSEDEADYYC | (SEQ ID |
| | (SEQ ID NO: 630) | | (SEQ ID NO: 632) | NO: 633) |
| S1-RBD- | QPVLTQPHSVSE | VQWYQQRPGSA | QRPSGVPDRFSG | FGGGTKLT |
| R3-T1- | SPGKTVTISCTR | PTTVIY | SIDSSSNSASLIIS | VL |
| C2 | S | (SEQ ID NO: 635) | GLMTEDEADYY | (SEQ ID |
| | (SEQ ID NO: 634) | | C | NO: 637) |
| | | | (SEQ ID NO: 636) | |
| S1-RBD- | QPGLTQPPSVSV | VHWYQQKAGQ | GRPSGIPERFSGS | FGPGTRLS |
| R3-T1- | APGQTATITCGG | APVLVVY | NSGNTATLTISR | VL |
| H8 | D | (SEQ ID NO: 639) | VEAGDEADYYC | (SEQ ID |
| | (SEQ ID NO: 638) | | (SEQ ID NO: 640) | NO: 641) |
| S1-RBD- | NFMLTQPHSVS | VQWYQQRPGSA | QRPSGVPDRFSG | FGGGTKLT |
| R3-E1- | ESPGKTVLISCT | PTTVIY | SIDSSSNSASLTIS | VL |
| E8 | RS | (SEQ ID NO: 643) | GLKTEDEADYY | (SEQ ID |
| | (SEQ ID NO: 642) | | C | NO: 645) |
| | | | (SEQ ID NO: 644) | |
| RBD-R3- | EIVLTQSPATLS | LPWYQQKPGQA | TRATGIPPRFSGS | FDGGTNVE |
| T1-H2 | VSPGERATLSCR | PRLLMY | GSGTEFSLTISSL | IK |
| | AS | (SEQ ID NO: 647) | QSEDFAVYYC | (SEQ ID |
| | (SEQ ID NO: 646) | | (SEQ ID NO: 648) | NO: 649) |
| S1-RBD- | NFMLTQPHSVS | VQWYQQRPGSF | QRPSGVPYRFSG | FGGGTKLT |
| R3-T1- | ESPGKTITISCTR | PITVIY | SIDRSSNSAALTI | VL |
| B7 | T | (SEQ ID NO: 651) | SDLKTEDEADYY | (SEQ ID |
| | (SEQ ID NO: 650) | | C | NO: 653) |
| | | | (SEQ ID NO: 652) | |
| S1-RBD- | QSALTQPASVS | VSWYQQHPGKA | KRPSGVPDRFSG | FGTGTKVT |
| R3-E1- | GSPGQSITISCTG | PKLMIY | SKSGNTASLTISG | VL |
| E5 | T | (SEQ ID NO: 655) | LQAEDEADYYC | (SEQ ID |
| | (SEQ ID NO: 654) | | (SEQ ID NO: 656) | NO: 657) |
| S1-R3- | SYELTQPPSVSV | VHWYQQKSGQA | DRPSGIPERFSGS | FGGGTKLT |
| T1-H6 | APGQTARITCG | PVLVVY | NSGNTATLTISR | VL |
| | GD | (SEQ ID NO: 659) | VEAGDEADYYC | (SEQ ID |
| | (SEQ ID NO: 658) | | (SEQ ID NO: 660) | NO: 661) |
| S1-RBD- | SSELTQDPAVSV | ASWYQQKPGQA | NRPSGIPDRFSGS | FGGGTKLA |
| R3-E1- | ALGQTVKITCQ | PVRVIY | SSGNTASLTITGA | VL |
| C6 | GD | (SEQ ID NO: 663) | QAEDEADYYC | (SEQ ID |
| | (SEQ ID NO: 662) | | (SEQ ID NO: 664) | NO: 665) |
| S1-RBD- | QSALTQPPSASG | VSWYQHHPDKA | NRPSGVSSRFSG | FGTGTEVT |
| R3-E1- | SPGQSLTISCTG | PKLLIY | SKSGNTASLTISG | PR |
| F2 | T | (SEQ ID NO: 667) | LQAEDEADYYC | (SEQ ID |
| | (SEQ ID NO: 666) | | (SEQ ID NO: 668) | NO: 669) |
| S1-RBD- | LPVLTQPPSVSV | VHWYQQKPGQA | DRPSGIPERFSGS | FGGGTTLT |
| R3-T1- | APGQTASITCGG | PLLVIY | SSGNTATLTISRV | VL |
| C3 | D | (SEQ ID NO: 671) | EAGDEADYYC | (SEQ ID |
| | (SEQ ID NO: 670) | | (SEQ ID NO: 672) | NO: 673) |
| S1-RBD- | QSALTQPRSVSG | VSWYQQHPGKA | KRPSGVPDRFSG | FGGGTKLT |
| R3-T1- | SPGQSVTISCTG | PKLIIY | SKSGNTASLTVS | VL |
| G12 | T | (SEQ ID NO: 675) | GLQAEDEADYY | (SEQ ID |
| | (SEQ ID NO: 674) | | C | NO: 677) |
| | | | (SEQ ID NO: 676) | |
| S1-RBD- | QSVLTQPPSASG | VSWYQQHPGKA | RRPSGVSSRFSGS | FGTGTKVT |
| R3-E1- | SPGQSVTISCTG | PKLMIY | KSGNTASLTISGL | VL |
| F1 | T | (SEQ ID NO: 679) | QAEDEADYYC | (SEQ ID |
| | (SEQ ID NO: 678) | | (SEQ ID NO: 680) | NO: 681) |
| S1-RBD- | NFMLTQPHSVS | VQWYQQRPGSA | QRPSGVPDRFSG | FGGGTKLT |
| R3-E1- | ESPGKTVTISCT | PTTVIY | SIDSSSNSASLTIS | VL |
| H8 | GS | (SEQ ID NO: 683) | GLKTEDEADYY | (SEQ ID |
| | (SEQ ID NO: 682) | | C | NO: 685) |
| | | | (SEQ ID NO: 684) | |
| S1-RBD- | QPGLTQTPSPSG | VTWYQQLPGTA | QRPSGVPDRFSA | FGGGTKM |
| R3-T1- | TPGQRVTISCSG | PKLLIY | SRSGTSPSLAITG | TVL |
| F1 | S | (SEQ ID NO: 687) | LQAEDEADYYC | (SEQ ID |
| | (SEQ ID NO: 686) | | (SEQ ID NO: 688) | NO: 689) |
| S1-R3- | LPVLTQPPSASG | VNWYHQLPRTA | HRPSGVPDRFSG | FGSGTKVT |
| T1-B10 | TPGQRVTISCSG | PKLLIY | S*SGTSASLAITV | VL |
| | S | (SEQ ID NO: 691) | IQSEDEADYYC | (SEQ ID |
| | (SEQ ID NO: 690) | | (SEQ ID NO: 692) | NO: 693) |
| RBD-R3- | LPVLTQPPSASG | VNWYHYLPRTA | HRASRVPDRFSG | FGSGTKVT |
| E1-D12 | TPGQRVTISCSG | PKLLIY | S*SGTSASLAITV | VL |
| | S | (SEQ ID NO: 695) | IQSEDEADYYC | (SEQ ID |
| | (SEQ ID NO: 694) | | (SEQ ID NO: 696) | NO: 697) |
| RBD-R3- | LPVLTQPPSASG | VNWYQQLPGTA | QRPSRVPDRFSG | FGSGTKSP |
| T1-C5 | TPGQRVTISCSG | PKLLIY | SKTGTSPSLAISV | SY |
| | S | (SEQ ID NO: 699) | LOSEDEADYYC | (SEQ ID |
| | (SEQ ID NO: 698) | | (SEQ ID NO: 700) | NO: 701) |
| S1-RBD- | QPVLTQPPSASG | VNWYQQLPGTA | QRPSGVPDRFSG | FGGGTKLT |
| R3-T1- | TPGQRVTISCSG | PKLLIY | SKSGTSASLAISG | VL |
| B4 | S | (SEQ ID NO: 703) | LQSEDEADYYC | (SEQ ID |
| | (SEQ ID NO: 702) | | (SEQ ID NO: 704) | NO: 705) |
| S1-RBD- | SSELTQDPAVSV | ASWYQQKPGQA | NRPSGIPDRFSGS | FGGGTKLT |
| R3-E1- | ALGQTVRITCQ | PVLVIY | SSGNTASLTITGA | VL |
| E7 | GD | (SEQ ID NO: 707) | QAEDEADYYC | (SEQ ID |
| | (SEQ ID NO: 706) | | (SEQ ID NO: 708) | NO: 709) |
| S1-RBD- | QPVLTQPPSASG | VNWYQQLPGTA | QRYPGVPDRFSG | FGGGTKLT |
| R3-T1- | TPGQRVTISCSG | PKLLIY | SKSGTSASLAISG | VL |
| C8 | S | (SEQ ID NO: 711) | LRSEDEADYYC | (SEQ ID |
| | (SEQ ID NO: 710) | | (SEQ ID NO: 712) | NO: 713) |
| S1-RBD- | QPGLTQPPSASG | VHWYQQLPGTA | QRPSGVPDRFSG | FGGGTKLT |
| R3-T1- | TPGQGVTISCSG | PKLLIY | SKSGTSASLAISG | VL |
| D7 | S | (SEQ ID NO: 715) | LRSEDEADYYC | (SEQ ID |
| | (SEQ ID NO: 714) | | (SEQ ID NO: 716) | NO: 717) |
| RBD-R3- | NFMLTQPHSVS | VQWLQQRLGSA | QRPSGVPDRFSG | FGGGTKLT |
| E1-F5 | ESPGKTVTISCT | PTTVIY | SIDSSSNSASLTIS | VL |
| | GS | (SEQ ID NO: 719) | GLKTEDEADYY | (SEQ ID |
| | (SEQ ID NO: 718) | | C | NO: 721) |
| | | | (SEQ ID NO: 720) | |
| Ab_13 | NFMLTQPHSVS | VQWYQQRPGSA | QRPSGVPDRFSG | FGGGTKLT |
| | ESPGKTVTISCT | PTTVIY | SIDSSSNSASLTIS | VL |
| | RS | (SEQ ID NO: 915) | GLKTEDEADYY | (SEQ ID |
| | (SEQ ID NO: | | C | NO: 917) |
| | 914) | | (SEQ ID NO: 916) | |
| Ab_14 | QSVLTQPPSASG | VSWYQHHPGKA | KRPSGVPDRFSG | FGGGTKLT |
| | SPGQSVTISCTG | PKLIIY | SKSGNTASLTVS | VL |
| | T | (SEQ ID NO: 919) | GLQADDEADYY | (SEQ ID |
| | (SEQ ID NO: | | C | NO: 921) |
| | 918) | | (SEQ ID NO: 920) | |
| Ab_15 | NFMLTQPHSVS | VQWYQQRPGSA | QRPSGVPDRFSG | FGTGTKVT |
| | ESPGKTVTISCT | PTTVIY | SIDRSSNSASLTIS | VL |
| | RS | (SEQ ID NO: 923) | GLTPDDEADYY | (SEQ ID |
| | (SEQ ID NO: 922) | | C | NO: 925) |
| | | | (SEQ ID NO: 924) | |
| Ab_16 | QSVLTQPPSVSG | VHWYQQVPGAA | NRPSGVPDRFSG | FGTGTKVT |
| | SPGQRVTMSCT | PRLLIY | SKSGTSASLTITG | VL |
| | GS | (SEQ ID NO: 927) | LQAEDEADYYC | (SEQ ID |
| | (SEQ ID NO: 926) | | (SEQ ID NO: 928) | NO: 929) |
| Ab_17 | QSALTQPASVS | VSWYQQHPGKA | KRPSGVPDRFSG | FGGGTKLT |
| | GSPGQSITISCTG | PKVMIY | SKSGNTASLTISG | VL |
| | T | (SEQ ID NO: 931) | LQAEDEADYYC | (SEQ ID |
| | (SEQ ID NO: 930) | | (SEQ ID NO: 932) | NO: 933) |
| Ab_18 | NFMLTQPHSMS | VQWYQQRPGSA | QRPSGVPDRFSG | FGGGTRLT |
| | GSAGKTVTVSCI | PTTVIY | SIDSSSNSASLTIS | VL |
| | RS | (SEQ ID NO: 935) | GLKTEDEADYY | (SEQ ID |
| | (SEQ ID NO: 934) | | C | NO: 937) |
| | | | (SEQ ID NO: 936) | |
| Ab_19 | QPGLTQPPAAS | VSWFKQFPETAP | HRPSGVPDRVSG | FGSGTKVT |
| | GTPGQRVTVSC | RLLIS | SKSGTSASLTISG | VL |
| | SGA | (SEQ ID NO: 939) | LQSDDEADYYC | (SEQ ID |
| | (SEQ ID NO: 938) | | (SEQ ID NO: 940) | NO: 941) |
| Ab_20 | NFMLTQPHSVS | VQWYQQRPGSA | QRPSGVP.DRFSG | FGGGTKLT |
| | ESPGKTVTISCT | PTTVIY | SIDSSSNSASLLIS | VL |
| | GS | (SEQ ID NO: 943) | GLKTEDEADYY | (SEQ ID |
| | (SEQ ID NO: 942) | | C | NO: 945) |
| | | | (SEQ ID NO: 944) | |
| Ab_21 | QSALTQPRSVSG | VSWYQQHPGKA | KRPSGVPDRFSG | FGGGTKLT |
| | SPGQSVTISCTG | PKLIIY | SKSGNTASLTVS | VL |
| | T | (SEQ ID NO: 947) | GLQAEDEADYY | (SEQ ID |
| | (SEQ ID NO: 946) | | C | NO: 949) |
| | | | (SEQ ID NO: 948) | |
| Ab_22 | NFMLTQPHSVS | VQWYRQRPGSA | QRPAGVPDRFSG | FGTGTKLT |
| | ESPGKTVIISCTR | PTTVIY | SVDSSSNSASLTI | VL |
| | T | (SEQ ID NO: 951) | TGLKTEDEADY | (SEQ ID |
| | (SEQ ID NO: 950) | | YC | NO: 953) |
| | | | (SEQ ID NO: 952) | |
| Ab_23 | QSALTQPPSASG | VSWYQQVPGKA | NRPSGVSSRFSG | FGGGTKLT |
| | SPGQSVTISCTG | PKLIIY | SKSGNTASLTISG | VL |
| | T | (SEQ ID NO: 955) | LQAEDEADYYC | (SEQ ID |
| | (SEQ ID NO: 954) | | (SEQ ID NO: 956) | NO: 957) |
| Ab_24 | QPVLTQPPSASG | VDWYHQVPGTA | ERSSGVPDRFSA | FGGGTKLT |
| | TPGQRVSISCSG | PQLLIY | SRSGNTASLTIIG | VL |
| | S | (SEQ ID NO: 959) | LQPEDEADYYC | (SEQ ID |
| | (SEQ ID NO: 958) | | (SEQ ID NO: 960) | NO: 961) |
| Ab_25 | NFMLTQPHSVS | VQWYQQRPGSA | QRPSGVPDRFSG | FGGGTKLT |
| | ESPGKTVTISCT | PTTVIY | SIGSSSNSASLTIS | VL |
| | GS | (SEQ ID NO: 963) | GLKTEDEADYY | (SEQ ID |
| | (SEQ ID NO: 962) | | C | NO: 965) |
| | | | (SEQ ID NO: 964) | |
| Ab_26 | LPVLTQPPSVSV | VHWYQQKPGQA | DRPSGIPERFSGS | FGGGTTLT |
| | APGQTASITCGG | PLLVIY | SSGNTATLTISRV | VL |
| | D | (SEQ ID NO: 967) | EAGDEADYYC | (SEQ ID |
| | (SEQ ID NO: 966) | | (SEQ ID NO: 968) | NO: 969) |
| Ab_27 | QSALTQPPSVSG | VSWYQQPPGKA | NRPSGVSNRFSG | FGTGTKVT |
| | SPGQSVTISCTG | PKLMIY | SKSGNTASLTISG | VL |
| | T | (SEQ ID NO: 971) | LQAEDEADYYC | (SEQ ID |
| | (SEQ ID NO: 970) | | (SEQ ID NO: 972) | NO: 973) |
| Ab_28 | NFMLTQPHSVS | VQWYQQRPGSA | QRPSGVPDRFSG | FGGGTKLT |
| | ESPGKTVTISCT | PTTVIY | SIDSSSNSASLTIS | VL |
| | GS | (SEQ ID NO: 975) | GLKTEDEADYY | (SEQ ID |
| | (SEQ ID NO: 974) | | C | NO: 977) |
| | | | (SEQ ID NO: 976) | |
| Ab_38 | EIVLTQSPATLS | LAWYQQKPGQA | TRATGIPARFSGS | FGGGTKVE |
| | VSPGERATLSCR | PRLLMY | GSGTEFSLTISSL | IK |
| | AS | (SEQ ID NO: 994) | QSEDFAVYYC | (SEQ ID |
| | (SEQ ID NO: 993) | | (SEQ ID NO: 995) | NO: 996) |
| wcS2- | NFMLTQPHSVS | VQWYQQRPGSA | QRPSGVSGRFSG | FGGGTKLT |
| T4-E7 | ESPGKTVTMSC | PTTVIY | SIDSSSNSASLTIS | VL |
| | TRS | (SEQ ID NO: 615) | GLQPEDEADYY | (SEQ ID NO: |
| | (SEQ ID NO: | | C | 549) |
| | 1419) | | (SEQ ID NO: 1495) | |
| wcS2- | QPVLTQPPSASG | VSWYQQHPDKA | KRPSGVPDRFSG | FGGGTKVT |
| E1-A9 | SPGQSVTISCTG | PKLLIY | SKSGNTASLTVS | VL |
| | T | (SEQ ID NO: 1458) | GLQADDEADYY | (SEQ ID NO: |
| | (SEQ ID NO: | | C | 593) |
| | 1420) | | (SEQ ID NO: 920) | |
| wcS2- | ETTLTQSPATLS | LAWYQQKPGQA | SRATGIPDRFSGS | FGGGTKVE |
| T4-H8 | LSPGERATLSCR | PRLLIY | GSGTDFTLTISRL | IK |
| | AS | (SEQ ID NO: 1459) | EPEDFAVYYC | (SEQ ID NO: |
| | (SEQ ID NO: | | (SEQ ID NO: 1496) | 996) |
| | 1421) | | | |
| wcS2- | QPVLTQPRSVSG | VSWYQHHPGKA | ERPSGVSSRFSGS | FGTGTRVA |
| T3-F5 | SPGQSVTISCTG | PKLMIY | KSGNTASLTISGL | VL |
| | T | (SEQ ID NO: 1460) | QGEDEADYFC | (SEQ ID NO: |
| | (SEQ ID NO: | | (SEQ ID NO: 1497) | 1542) |
| | 1422) | | | |
| wcS2- | QSVLTQPPSASG | VSWYQQHPGKA | KRPSGVPDRFSG | FGTGTKVT |
| E2-C1 | SPGQSVTISCTG | PKPMIY | SKSGNTASLTVS | VL |
| | T | (SEQ ID NO: 1461) | GLQAEDEADYY | (SEQ ID NO: |
| | (SEQ ID NO: 574) | | C | 573) |
| | | | (SEQ ID NO: 676) | |
| wcS2- | EIVLTQSPATLS | LAWYQQKPGQA | SRANGIPDRFSGS | FGGGTKLE |
| T4-C9 | LSPGERATLSCR | PRLLIN | GSGTDFTLTITRL | IK |
| | AS | (SEQ ID NO: 1462) | EPEDFAVYFC | (SEQ ID NO: |
| | (SEQ ID NO: 602) | | (SEQ ID NO: 1498) | 1543) |
| wcS2- | SSELTQDPAVSV | ASWYQQKPGQA | NRPSGIPDRFSGS | FGGGTKLT |
| T3-B9 | ALGQTVRITCQ | PLLVIY | SSGNTASLTITGA | VL |
| | GD | (SEQ ID NO: 1463) | QAEDEADYYC | (SEQ ID NO: |
| | (SEQ ID NO: 706) | | (SEQ ID NO: 664) | 549) |
| wcS2- | QSVLTQPLSASG | VNWYQQVPGTA | RRPSGVPDRFSG | FGTGTKVT |
| E3-C8 | TPGHRVTISCSG | PKLLIY | SKSGTSAALAIS | VL |
| | S | (SEQ ID NO: 1464) | GLQSEDEADYY | (SEQ ID NO: |
| | (SEQ ID NO: | | C | 573) |
| | 1423) | | (SEQ ID NO: 1499) | |
| wcS2- | NFMLTQPHSVS | VQWYQQRPGSA | QRPSGVPDRFSG | FGGGTKLT |
| T4-F8 | ESPGKTVTISCT | PTAVIF | SIDSSSNSASLTIS | VL |
| | RS | (SEQ ID NO: 1465) | GLRTEDEADYY | (SEQ ID NO: |
| | (SEQ ID NO: 594) | | C | 549) |
| | | | (SEQ ID NO: 1500) | |
| wcS2- | LPVLTQPPSASG | VNWYQQLPGTA | QRPSGVPDRFSG | FGGGTNLA |
| T3-F1 | TPGQRVTISCSG | PKLLIY | SKSGTSASLAISG | VL |
| | S | (SEQ ID NO: 543) | LRSEDEADYYC | (SEQ ID NO: |
| | (SEQ ID NO: 690) | | (SEQ ID NO: 632) | 1544) |
| wcS2- | SSELTQDPAVSV | ASWYQQKPGQA | NRPSGIPDRFSGS | FGTGTKVT |
| E2-B1 | ALGQTVRITCQ | PVLVIY | SSGNTASLTITGA | VL |
| | GD | (SEQ ID NO: 707) | QAEDEADYYC | (SEQ ID NO: |
| | (SEQ ID NO: 706) | | (SEQ ID NO: 664) | 573) |
| wcS2- | SYELTQPPSVSQ | AAWLQQHQGHP | IRPSGISERLSAST | FGEGTKLT |
| T2-G3 | GLRQTATLTCT | PKLLSY | SGNTASLTITGL | VL |
| | GN | (SEQ ID NO: 611) | QPEDEADYYC | (SEQ ID NO: |
| | (SEQ ID NO: | | (SEQ ID NO: 1501) | 1545) |
| | 1424) | | | |
| wcS2- | QSVLTQPLSASG | VNWYQQVPGTA | RRPSGVPDRFSG | FGTGTKVT |
| T1-A6 | TPGHRVTISCSG | PKLLIY | SKSGTSAALAIS | VL |
| | S | (SEQ ID NO: 1464) | GLQSEDEADYY | (SEQ ID NO: |
| | (SEQ ID NO: | | C | 573) |
| | 1423) | | (SEQ ID NO: 1499) | |
| wcS2- | NFMLTHPHSVS* | VQWYQKRPGSA | QRPAGVSGRFSG | FGGGTNLT |
| T4-D4 | SPGKTATMSCT | PTTVIY | SIDSSSNSASLTIS | VL |
| | RS | (SEQ ID NO: 1466) | AVHPEDEADYY | (SEQ ID NO: |
| | (SEQ ID NO: | | C | 1546) |
| | 1425) | | (SEQ ID NO: 1502) | |
| wcS2- | HFVLTQPPSASE | DDCCHQRPPGAP | HKPSAGSADLFA | FGAGTKVT |
| T2-D10 | SPGKTVTMSCT | PTAMN | AFDSASTSALIAF | VL |
| | GT | (SEQ ID NO: 1467) | SVLHADDDDDY | (SEQ ID NO: |
| | (SEQ ID NO: | | CC | 1547) |
| | 1426) | | (SEQ ID NO: 1503) | |
| wcS2- | ETTLTQSPGTLS | LAWYQQKPGQA | NRAPGIPARFSGS | FGPGTKVH |
| T1-G9 | LSPGERATLSCR | PRLLIY | GSGTDFTLTISSL | IK |
| | AS | (SEQ ID NO: 1459) | EPEDFAVYYC | (SEQ ID NO: |
| | (SEQ ID NO: | | (SEQ ID NO: 1504) | 1548) |
| | 1427) | | | |
| wcS2- | EIVLTQSPATLA | LAWYQQRPGQA | SRANGIPYRFSGS | SAERTKME |
| E3-H7 | LSPGERATLSCR | PRLLIN | GSGTDFTLTITRL | IK |
| | DS | (SEQ ID NO: 1468) | EPEDFAVYFC | (SEQ ID NO: |
| | (SEQ ID NO: | | (SEQ ID NO: 1505) | 1549) |
| | 1428) | | | |
| wcS2- | SSELTQDPAVSV | ANWYQQKPGQA | ERPSGIPERFSGS | FGGGTKLT |
| T2-C11 | ALGQTVRITCQ | PILVMS | SSGTRVTLTISGV | VL |
| | GD | (SEQ ID NO: 1469) | QAEDEADYYC | (SEQ ID NO: |
| | (SEQ ID NO: 706) | | (SEQ ID NO: 1506) | 549) |
| SARS2- | QSALTQPASVS | VSWYQQHPGKA | KRPSGVPDRFSG | FGTGTKVA |
| R3-G2- | GSPGQSITISCTG | PKLMIY (SEQ ID | SKSGNTASLTVS | VL (SEQ ID |
| P1- | T (SEQ ID NO: | NO: 627) | GLQAEDEADYY | NO: 1550) |
| H9_PelB- | 654) | | C (SEQ ID NO: | |
| F_2020- | | | 676) | |
| 06- | | | | |
| 19_D08 | | | | |
| SARS2- | QSALTQPASVS | VSWYQQHPGKA | NRPSGVSNRFSG | FGAGTKVT |
| R3-G3- | GSPGQSITISCTG | PKLMIS (SEQ ID | SKSGNTASLTISG | VL (SEQ ID |
| P1- | T (SEQ ID NO: | NO: 1470) | LQAEDEADYYC | NO: 1547) |
| F8_PelB- | 654) | | (SEQ ID NO: 588) | |
| F_2020- | | | | |
| 06- | | | | |
| 19_F11 | | | | |
| SARS2- | EIVLTQSPATLS | LAWYQQKPGQA | TRATGIPARFSGS | FGQGTRVE |
| R3-G2- | VSPGERATLSCR | PRLLIY (SEQ ID | GSGTEFTLTVSR | IR (SEQ ID |
| P1- | AS (SEQ ID NO: | NO: 1459) | LEPEDFAVYYC | NO: 1551) |
| B4_PelB- | 646) | | (SEQ ID NO: | |
| F_2020- | | | 1507) | |
| 06- | | | | |
| 19_B02 | | | | |
| SARS2- | QSVLTQPPSASG | VNWYQQLPGTA | QRPSGVPDRFSG | FGGGTNLA |
| R3-G3- | TPGQRVTISCSG | PKLLIY (SEQ ID | SKSGTSVSLAISG | VL (SEQ ID |
| P1- | S (SEQ ID NO: | NO: 543) | LQSEDEADYYC | NO: 1544) |
| B2_PelB- | 1429) | | (SEQ ID NO: | |
| F_2020- | | | 1508) | |
| 06- | | | | |
| 19_A08 | | | | |
| SARS2- | SYELTQPPSVSV | VHWYQQKPGQA | DRPPGIPERFSGS | FGTGTKVT |
| R3-G3- | APGKTANMTCG | PVLVVY (SEQ ID | NSGNTATLTISR | VL (SEQ ID |
| P1- | GN (SEQ ID NO: | NO: 1471) | VEVGDEADYYC | NO: 573) |
| B3_PelB- | 1430) | | (SEQ ID NO: | |
| F_2020- | | | 1509) | |
| 06- | | | | |
| 19_B08 | | | | |
| SARS2- | SSELTQDPAVSV | ASWYQQKPGQA | NRPSGIPDRISGS | FGTGTKVT |
| R3-G1- | ALGQTVRITCQ | PVLVIY (SEQ ID | SSGNTASLTITGA | VL (SEQ ID |
| P3- | GD (SEQ ID NO: | NO: 707) | QAEDEADYYC | NO: 573) |
| E8_PelB- | 706) | | (SEQ ID NO: | |
| F_2020- | | | 1510) | |
| 06- | | | | |
| 19_H04 | | | | |
| SARS2- | QSGLTQPPSVSG | VHWYQQLPGTA | NRPSGVPDRFSG | FGGGTKLT |
| R3-G1- | APGQRVTISCTG | PKLIIY (SEQ ID | SKSGTSASLAITG | VL (SEQ ID |
| P2- | S (SEQ ID NO: | NO: 1472) | LQAEDEADYYC | NO: 549) |
| D7_PelB- | 1431) | | (SEQ ID NO: | |
| F_2020- | | | 1511) | |
| 06- | | | | |
| 19_H09 | | | | |
| SARS2- | SYELTQPPSVSV | AYWYQQRPGQA | ERPSGIPERFSGS | FGGGTKVT |
| R3-G1- | SPGQTARITCSG | PVLVIY (SEQ ID | SSGTTVTLTISGV | VL (SEQ ID |
| P1- | D (SEQ ID NO: | NO: 1473) | QAEDEADYYC | NO: 593) |
| C6_PelB- | 1432) | | (SEQ ID NO: | |
| F_2020- | | | 1512) | |
| 06- | | | | |
| 19_H02 | | | | |
| SARS2- | HTVLTHPAPAA | VSWYQQHPGKA | NRPSGVPDRFSG | S*TGTEVA |
| R3-T-P2- | AYPGQTITISCS | PTLVIY (SEQ ID | SIYGNTDSLTVS | FI (SEQ ID |
| E12_PelB- | AT (SEQ ID NO: | NO: 1474) | AGVEDEDDYYC | NO: 1552) |
| F_2020- | 1433) | | (SEQ ID NO: | |
| 06- | | | 1513) | |
| 19_D07 | | | | |
| SARS2- | DIVMTQTPSTLS | VAWYQQKPGKV | RLEPGVPSRFSGS | FGQGTKV |
| R3-G2- | ASVGDRVTISCR | PELLMY (SEQ ID | GSGTEFTLTISNL | DIK (SEQ |
| P1- | AS (SEQ ID NO: | NO: 1475) | QPEDFATYYC | ID NO: |
| D12_PelB- | 1434) | | (SEQ ID NO: | 1553) |
| F_2020- | | | 1514) | |
| 06- | | | | |
| 19_H04 | | | | |
| SARS2- | NFMLTQPHSVS | VQWYQQRPGSS | QRPSEVPDRFSG | FGGGTKLT |
| R3-G1- | ESPGKTVTISCT | PTAVIY (SEQ ID | SIDISSNSASLTIS | VL (SEQ ID |
| P2- | RS (SEQ ID NO: | NO: 1476) | GLKTEDEADYY | NO: 549) |
| E8_PelB- | 594) | | C (SEQ ID NO: | |
| F_2020- | | | 1515) | |
| 06- | | | | |
| 19_E10 | | | | |
| SARS2- | ETTLTQSPGTLS | LAWYQQKPGQA | TRATGIPARFSGS | FGQGTKLE |
| R3-G1- | LSPGERATLSCR | PRLLIY (SEQ ID | GSGTDFTLTISRL | IK (SEQ ID |
| P1- | AS (SEQ ID NO: | NO: 1459) | EPEDFAVYFC | NO: 1554) |
| A10_PelB- | 1427) | | (SEQ ID NO: | |
| F_2020- | | | 1516) | |
| 06- | | | | |
| 19_H01 | | | | |
| SARS2- | QSALTQPPSASG | VSWYQQPPGTA | NRPSGVPDRFSG | FGTGTKVT |
| R3-G1- | SPGQSVTISCTG | PKLMIY (SEQ ID | SKSGNTASLTISG | VL (SEQ ID |
| P3- | T (SEQ ID NO: | NO: 1477) | LQAEDEADYYC | NO: 573) |
| B5_PelB- | 606) | | (SEQ ID NO: | |
| F_2020- | | | 1517) | |
| 06- | | | | |
| 19_D02 | | | | |
| SARS2- | NFMLTQPHSVS | VQWYQQRPGSS | RRPSGVPDRFSG | FGGGTKLT |
| R3-G3- | GSPGKTVTISCT | PTTVIY (SEQ ID | SVDRSSNSASLTI | VL (SEQ ID |
| P1- | RD (SEQ ID NO: | NO: 547) | SGLDTEDEADYY | NO: 549) |
| A2_PelB- | 1435) | | C (SEQ ID NO: | |
| F_2020- | | | 1518) | |
| 06- | | | | |
| 19_B07 | | | | |
| SARS2- | QPVLTQPPSVSG | VQWYQQVPGTA | NRPSGVPDRFSG | FGTGTKVN |
| R3-G3- | APGQSVTISCIG | PKLLIY (SEQ ID | SKSGTSASLAISG | VL (SEQ ID |
| P1- | S (SEQ ID NO: | NO: 1478) | LRSEDEADYFC | NO: 1555) |
| D1_PelB- | 1436) | | (SEQ ID NO: | |
| F_2020- | | | 1519) | |
| 06- | | | | |
| 19_G09 | | | | |
| SARS2- | QPGLTQPPSVSK | AAWLQQHQGHP | NRPSGISERFSAS | FGGGSRLT |
| R3-G1- | DLRQTATLTCT | PKLLSY (SEQ ID | TSGNTASLTITGL | VL (SEQ ID |
| P2- | GN (SEQ ID NO: | NO: 611) | QPEDEADYYC | NO: 1556) |
| D4_PelB- | 1437) | | (SEQ ID NO: | |
| F_2020- | | | 1520) | |
| 06- | | | | |
| 19_G09 | | | | |
| SARS2- | QPVLTQPPSASG | VNWYQQLPGTA | HRPSGVPDRFSG | FGGGTKLT |
| R3-G1- | TPGQRVTISCSG | PKLLIY (SEQ ID | SKSGNTASLTISG | VL (SEQ ID |
| P1- | G (SEQ ID NO: | NO: 543) | LQAEDEADYYC | NO: 549) |
| B6_PelB- | 1438) | | (SEQ ID NO: | |
| F_2020- | | | 1521) | |
| 06- | | | | |
| 19_E02 | | | | |
| SARS2- | QPVLTQPPSVSV | ASWYQQKPGQS | KRPSGIPERFSGS | FGTGTKVT |
| R3-G3- | SPGQTASITCSG | PVLVIY (SEQ ID | NSGNTATLTISET | VL (SEQ ID |
| P1- | D (SEQ ID NO: | NO: 1479) | QAMDEADYYC | NO: 573) |
| G8_PelB- | 1439) | | (SEQ ID NO: | |
| F_2020- | | | 1522) | |
| 06- | | | | |
| 25_A05 | | | | |
| SARS2- | SYELTQPPSVSV | AYWYRQRPGQA | ERPSGIPERFSGS | FGGGTKLT |
| R3-G1- | SPGQTATITCSG | PVLVIY (SEQ ID | SSGTTVTLTISGV | VL (SEQ ID |
| P1- | D (SEQ ID NO: | NO: 1480) | QAEDEADYYC | NO: 549) |
| F11_PelB- | 1440) | | (SEQ ID NO: | |
| F_2020- | | | 1512) | |
| 06- | | | | |
| 25_B05 | | | | |
| SARS2- | QSVLTQPPSASG | VSWYQQHPGKA | KRPSGVPDRFSG | FGTGTEVT |
| R3-T1- | SPGQSVTISCTG | PKVLIY (SEQ ID | SKSGNTASLTVS | VL (SEQ ID |
| P3- | T (SEQ ID NO: | NO: 1481) | GLQAEDEADYY | NO: 1557) |
| F4_PelB- | 574) | | C (SEQ ID NO: | |
| F_2020- | | | 676) | |
| 06- | | | | |
| 20_C03 | | | | |
| SARS2- | QSVLTQPPSASG | VSWYQHHPGKA | KRPSGVPDRFSG | FGTGTKVT |
| R3-G1- | SPGHSVTISCTG | PKLMIY (SEQ ID | SKSGNTASLSVS | VL (SEQ ID |
| P2- | T (SEQ ID NO: | NO: 1460) | GLQAEDEADYY | NO: 573) |
| B8_PelB- | 1441) | | C (SEQ ID NO: | |
| F_2020- | | | 1523) | |
| 06- | | | | |
| 25_C06 | | | | |
| SARS2- | NFMLTQPHSVS | VQWYQQRPGSS | QRPSGVPDRFSG | FGGGTKLT |
| R3-G1- | ESPGKTVAISCT | PTTVIY (SEQ ID | SIDSSSNSASLTIS | VL (SEQ ID |
| P2- | RS (SEQ ID NO: | NO: 547) | GLKTEDEADYY | NO: 549) |
| D3_PelB- | 1442) | | C (SEQ ID NO: | |
| F_2020- | | | 616) | |
| 06- | | | | |
| 25_C12 | | | | |
| SARS2- | LPMLTQPPSMS | VNWYQQVPGTA | QRPSGVPDRFSG | IGGGTKLT |
| R3-G1- | GTPGQRVTISCS | PKVLIY (SEQ ID | SKSGTSASLAISG | VL (SEQ ID |
| P4- | GS (SEQ ID NO: | NO: 1482) | LRSEDEADYSC | NO: 1558) |
| C5_PelB- | 1443) | | (SEQ ID NO: | |
| F_2020- | | | 1524) | |
| 06- | | | | |
| 25_C07 | | | | |
| SARS2- | SYELTQPPSVSK | ASWLQHHQGHP | NRPSGISERFSAS | FGTGTKVT |
| R3-G1- | GLRQTATLTCT | PKLLSY (SEQ ID | RSGNTASLTITGL | VL (SEQ ID |
| P2- | GN (SEQ ID NO: | NO: 1483) | QPEDEADYYC | NO: 573) |
| D6_PelB- | 1444) | | (SEQ ID NO: | |
| F_2020- | | | 1525) | |
| 06- | | | | |
| 25_D02 | | | | |
| SARS2- | SYELTQPPSVSV | VSWYQQKPGHS | KRPSGIPERFSGS | FGGGTKLA |
| R3-G1- | SPGQTATITCSG | PLLVIY (SEQ ID | NSGNRAILTINGT | VL (SEQ ID |
| P1- | D (SEQ ID NO: | NO: 1484) | QALDEADYYC | NO: 665) |
| B7_PelB- | 1440) | | (SEQ ID NO: | |
| F_2020- | | | 1526) | |
| 06- | | | | |
| 25_A03 | | | | |
| 95_PelB- | ETTLTQSPGTLS | LAWYQQKPGQA | NRATGIPDRFSG | FGQGTKLE |
| F_2020- | LSPGERATLSCR | PRLLIY (SEQ ID | SGSGTDFTLTISS | IN (SEQ ID |
| 06- | AS (SEQ ID NO: | NO: 1459) | LQPEDFATYYC | NO: 1559) |
| 25_G11 | 1427) | | (SEQ ID NO: | |
| | | | 1527) | |
| 73_PelB- | DIVMTQSPATLS | LAWYQQRPGQA | TRATDIPDRFTGS | FGQGTKVE |
| F_2020- | VSPGERATLSCR | PRLLIS (SEQ ID | GSGTDFTLTISSL | SK (SEQ ID |
| 06- | AS (SEQ ID NO: | NO: 1485) | EPEDFAVYYC | NO: 1560) |
| 25_F09 | 1445) | | (SEQ ID NO: | |
| | | | 1528 | |
| SARS2- | QSVLTQPPSASG | VSWYQQHPGKA | KRPSGVPDRFSA | FGGGTTLT |
| R3-G3- | SPGQSVTVSCTG | PKLLIH (SEQ ID | SKSGNTASLTISG | VL (SEQ ID |
| P1- | T (SEQ ID NO: | NO: 1486) | LQPEDEGDYFC | NO: 673) |
| G9_PelB- | 1446) | | (SEQ ID NO: | |
| F_2020- | | | 1529) | |
| 06- | | | | |
| 25_B05 | | | | |
| SARS2- | QPVLTQPPSVSK | TAWLQQHQGHP | NRPSGVSERFSA | FGGGTKLT |
| R3-G1- | DLRQTATLTCT | PKLVSY (SEQ ID | SRSGNTASLTITG | VL (SEQ ID |
| P3- | GN (SEQ ID NO: | NO: 1487) | LQAEDEADYYC | NO: 549) |
| G7_PelB- | 1447) | | (SEQ ID NO: | |
| F_2020- | | | 1530) | |
| 06- | | | | |
| 25_H01 | | | | |
| SARS2- | NFMLTQPHSVS | VQWYQQRPGSA | QRPSGVPDRFSG | FGGGTKLT |
| R3-G1- | ESPGKTVTISCT | PTTVIY (SEQ ID | SIDSSSKTASLIIS | VP (SEQ ID |
| P4- | RS (SEQ ID NO: | NO: 615) | GLETEDEADYYC | NO: 1561) |
| A2_PelB- | 594) | | (SEQ ID NO: | |
| F_2020- | | | 1531) | |
| 06- | | | | |
| 25_A06 | | | | |
| SARS2- | QSVLTQPPSVSG | VHWYQQLPGTA | NRPSGVPDRFSG | FGGGTKLT |
| R3-G3- | APGQRVTISCTG | PKLIIY (SEQ ID | SKSGTSASLAITG | VL (SEQ ID |
| P1- | S (SEQ ID NO: | NO: 1472) | LQAEDEADYYC | NO: 549) |
| G1_PelB | 1448) | | (SEQ ID NO: | |
| -F_2020- | | | 1511) | |
| 06- | | | | |
| 25_F04 | | | | |
| SARS2- | QSALTQPASVS | VSWYQQHPASA | NRPSGVSNRFSG | FGTGTKVT |
| R3-G3- | GSPGQSITISCTG | PKLIIY (SEQ ID | SESGDTASLTISG | VL (SEQ ID |
| P1- | T (SEQ ID NO: | NO: 1488) | LRAEDEADYFC | NO: 573) |
| F1_PelB- | 654) | | (SEQ ID NO: | |
| F_2020- | | | 1532) | |
| 06- | | | | |
| 25_C04 | | | | |
| SARS2- | LPVLTQPPSASE | VNWYQQLPGTA | QRPSGVPDRFFG | FGGGTKLT |
| R3-G1- | TPGQRVTISCSG | PKLLIY (SEQ ID | SKSGTSASLAISG | VL (SEQ ID |
| P3- | G (SEQ ID NO: | NO: 543) | LQSEDEADYYC | NO: 549) |
| H10_PelB- | 1449) | | (SEQ ID NO: | |
| F_2020- | | | 1533) | |
| 06- | | | | |
| 25_H10 | | | | |
| 85_PelB- | QPVLTQPPSVSG | VHWYQQLPGTA | QRPSGVPDRFSG | FGGGTKVT |
| F_2020- | APGQSVTISCTG | PKLLIY (SEQ ID | SKSGTSASLAISG | VL (SEQ ID |
| 06- | S (SEQ ID NO: | NO: 715) | LOSEDEADYYC | NO: 593) |
| 25_G10 | 1450) | | (SEQ ID NO: 544) | |
| SARS2- | QSVLTQPPSASG | VYWYQRLPGTA | QRPSGVPDRFSG | FGTGTKVT |
| R3-G1- | TPGGRVTISCSG | PKLLIY (SEQ ID | SKSGTSASLAISG | VL (SEQ ID |
| P4- | S (SEQ ID NO: | NO: 1489) | LQSEDEADYFC | NO: 573) |
| A3_PelB- | 1451) | | (SEQ ID NO: | |
| F_2020- | | | 1534) | |
| 06- | | | | |
| 25_B06 | | | | |
| SARS2- | SSELTQDPAVSV | ANWYQQKSGQA | IRPSIPDRFSGSSS | VRRRIKLT |
| R3-G1- | ALGQTVRIICQG | PILVMY (SEQ ID | GNTASLTITGAQ. | VL (SEQ ID |
| P3- | D (SEQ ID NO: | NO: 1490) | EDEADYYC (SEQ | NO: 1562) |
| H6_PelB- | 1452) | | ID NO: 1535) | |
| F_2020- | | | | |
| 06- | | | | |
| 25_H08 | | | | |
| SARS2- | QSALTQPASVS | VSWYQQHPGKA | YRPSGISHRFSGS | FGTGTKVT |
| R3-G3- | GSPGQSITISCTG | PKLMIY (SEQ ID | KSGNTASLTISGL | VL (SEQ ID |
| P1- | T (SEQ ID NO: | NO: 627) | QAEDEADYYC | NO: 573) |
| G5_PelB- | 654) | | (SEQ ID NO: | |
| F_2020- | | | 1536) | |
| 06- | | | | |
| 25_H04 | | | | |
| SARS2- | SYELTQPPSVSV | AYWYQQRPGQA | ERSSGIPERFSGS | FGGGTKLT |
| R3-G1- | SPGQTASITCSG | PVLLIY (SEQ ID | GSGTTVTLTING | VL (SEQ ID |
| P4- | D (SEQ ID NO: | NO: 1491) | VQAEDEADYYC | NO: 549) |
| C10_PelB- | 1453) | | (SEQ ID NO: | |
| F_2020- | | | 1537) | |
| 06- | | | | |
| 25_F07 | | | | |
| SARS2- | LPVLTQPPSVSG | VHWYQQLPGAA | NRPSGVPDRFSG | FGGGTRLT |
| R3-G1- | APGQRVAISCTG | PKLLIY (SEQ ID | SKSGTSASLAISG | VL (SEQ ID |
| P1- | S (SEQ ID NO: | NO: 1492) | LRSEDEADYYC | NO: 937) |
| F10_PelB- | 1454) | | (SEQ ID NO: | |
| F_2020- | | | 1538) | |
| 06- | | | | |
| 25_B04 | | | | |
| SARS2- | QSVLTQPPSVSV | VHWYQQKAGQ | DRPSGIPERFSGS | FGGGTKLT |
| R3-T1- | APGQTARITCG | APVLVVH (SEQ | NSGNTATLTISR | VL (SEQ ID |
| P4- | GD (SEQ ID NO: | ID NO: 1493) | VEAGDEADYYC | NO: 549) |
| H4_PelB- | 1455) | | (SEQ ID NO: 660) | |
| F_2020- | | | | |
| 06- | | | | |
| 20 H04 | | | | |
| SARS2- | DIVMTQSPLSLP | LDWYLQKPGQS | NRASGVPDRFSG | FGQGTRLD |
| R3-G2- | VTPGEPASISCR | PQLLIH (SEQ ID | SGSDTDFTLKISR | IK (SEQ ID |
| P1- | SS (SEQ ID NO: | NO: 1494) | VEAEDVGVYYC | NO: 1563) |
| E8_PelB- | 1456) | | (SEQ ID NO: | |
| F_2020- | | | 1539) | |
| 06- | | | | |
| 25_A02 | | | | |
| SARS2- | QSVLTQPPSVSG | VHWYQQLPGTA | IRPSGVPDRFSAS | FGGGTKLT |
| R3-G1- | APGERVTFSCTG | PKLLIY (SEQ ID | KSGTSASLAITGL | VL (SEQ ID |
| P1- | T (SEQ ID NO: | NO: 715) | QSEDEGDYYC | NO: 549) |
| C3_PelB- | 1457) | | SEQ ID NO: | |
| F_2020- | | | 1540) | |
| 06- | | | | |
| 25_A07 | | | | |
| SARS2- | QSVLTQPPSASG | VSWYQQHPGKA | KRPSGVPDRFSG | FGTGTEVT |
| R3-G3- | SPGQSVTISCTG | PKVLIY (SEQ ID | SKSGNTASLTVS | VL (SEQ ID |
| P1- | T (SEQ ID NO: | NO: 1481) | GLQAEDEADYY | NO: 1557) |
| G2_PelB- | 574) | | C (SEQ ID NO: | |
| F_2020- | | | 676) | |
| 06- | | | | |
| 25_G04 | | | | |
| SARS2- | NFMLTQPHSVS | VQWYQQRPGSA | QRPSGVPDRFSA | FGGGTKLT |
| R3-G1- | ESPGKTVTISCT | PTTVIY (SEQ ID | SIDSSSNSASLTIS | VL (SEQ ID |
| P4- | RS (SEQ ID NO: | NO: 615) | GLKAEDEADYY | NO: 549) |
| D1_PelB- | 594) | | C (SEQ ID NO: | |
| F_2020- | | | 1541) | |
| 06- | | | | |
| 25_A08 | | | | |

The asterisks noted in the tables herein are read as a Q (glutamine) in the amino acid sequences described in the tables herein.

Antibodies

As used herein, the term “antibody” can refer to immunoglobulin molecules and immunologically active portions of immunoglobulin (Ig) molecules, i.e., molecules that contain an antigen binding site that specifically binds (immunoreacts with) an antigen. By “specifically binds” or “immunoreacts with” is meant that the antibody reacts with one or more antigenic determinants of the desired antigen and does not react with other polypeptides. Antibodies can include, but are not limited to, polyclonal, monoclonal, and chimeric antibodies. In some embodiments, the antibodies described herein are directed to SARS-CoV2.

For example, the antibodies described herein are directed to SARS-CoV2 having NCBI Reference Sequence: NC_045512 (amino acid residues 1-7116; SEQ ID NO: 979):

MESLVPGFNEKTHVQLSLPVLQVRDVLVRGFGDSVEEVLSEARQHLKDGTCGLVEVEKGVLPQLEQP
YVFIKRSDARTAPHGHVMVELVAELEGIQYGRSGETLGVLVPHVGEIPVAYRKVLLRKNGNKGAGGH
SYGADLKSFDLGDELGTDPYEDFQENWNTKHSSGVTRELMRELNGGAYTRYVDNNFCGPDGYPLECI
KDLLARAGKASCTLSEQLDFIDTKRGVYCCREHEHEIAWYTERSEKSYELQTPFEIKLAKKFDTFNG
ECPNFVFPLNSIIKTIQPRVEKKKLDGFMGRIRSVYPVASPNECNQMCLSTLMKCDHCGETSWQTGD
FVKATCEFCGTENLTKEGATTCGYLPQNAVVKIYCPACHNSEVGPEHSLAEYHNESGLKTILRKGGR
TIAFGGCVFSYVGCHNKCAYWVPRASANIGCNHTGVVGEGSEGLNDNLLEILQKEKVNINIVGDFKL
NEEIAIILASFSASTSAFVETVKGLDYKAFKQIVESCGNFKVTKGKAKKGAWNIGEQKSILSPLYAF
ASEAARVVRSIFSRTLETAQNSVRVLQKAAITILDGISQYSLRLIDAMMFTSDLATNNLVVMAYITG
GVVQLTSQWLTNIFGTVYEKLKPVLDWLEEKFKEGVEFLRDGWEIVKFISTCACEIVGGQIVTCAKE
IKESVQTFFKLVNKFLALCADSIIIGGAKLKALNLGETFVTHSKGLYRKCVKSREETGLLMPLKAPK
EIIFLEGETLPTEVLTEEVVLKTGDLQPLEQPTSEAVEAPLVGTPVCINGLMLLEIKDTEKYCALAP
NMMVTNNTFTLKGGAPTKVTFGDDTVIEVQGYKSVNITFELDERIDKVLNEKCSAYTVELGTEVNEF
ACVVADAVIKTLQPVSELLTPLGIDLDEWSMATYYLFDESGEFKLASHMYCSFYPPDEDEEEGDCEE
EEFEPSTQYEYGTEDDYQGKPLEFGATSAALQPEEEQEEDWLDDDSQQTVGQQDGSEDNQTTTIQTI
VEVQPQLEMELTPVVQTIEVNSFSGYLKLTDNVYIKNADIVEEAKKVKPTVVVNAANVYLKHGGGVA
GALNKATNNAMQVESDDYIATNGPLKVGGSCVLSGHNLAKHCLHVVGPNVNKGEDIQLLKSAYENFN
QHEVLLAPLLSAGIFGADPIHSLRVCVDTVRTNVYLAVFDKNLYDKLVSSFLEMKSEKQVEQKIAEI
PKEEVKPFITESKPSVEQRKQDDKKIKACVEEVTTTLEETKFLTENLLLYIDINGNLHPDSATLVSD
IDITFLKKDAPYIVGDVVQEGVLTAVVIPTKKAGGTTEMLAKALRKVPTDNYITTYPGQGLNGYTVE
EAKTVLKKCKSAFYILPSIISNEKQEILGTVSWNLREMLAHAEETRKLMPVCVETKAIVSTIQRKYK
GIKIQEGVVDYGARFYFYTSKTTVASLINTLNDLNETLVTMPLGYVTHGLNLEEAARYMRSLKVPAT
VSVSSPDAVTAYNGYLTSSSKTPEEHFIETISLAGSYKDWSYSGQSTQLGIEFLKRGDKSVYYTSNP
TTFHLDGEVITFDNLKTLLSLREVRTIKVFTTVDNINLHTQVVDMSMTYGQQFGPTYLDGADVTKIK
PHNSHEGKTFYVLPNDDTLRVEAFEYYHTTDPSFLGRYMSALNHTKKWKYPQVNGLTSIKWADNNCY
LATALLTLQQIELKFNPPALQDAYYRARAGEAANFCALILAYCNKTVGELGDVRETMSYLFQHANLD
SCKRVLNVVCKTCGQQQTTLKGVEAVMYMGTLSYEQFKKGVQIPCTCGKQATKYLVQQESPFVMMSA
PPAQYELKHGTFTCASEYTGNYQCGHYKHITSKETLYCIDGALLTKSSEYKGPITDVFYKENSYTTT
IKPVTYKLDGVVCTEIDPKLDNYYKKDNSYFTEQPIDLVPNQPYPNASFDNFKFVCDNIKFADDLNQ
LTGYKKPASRELKVTFFPDLNGDVVAIDYKHYTPSFKKGAKLLHKPIVWHVNNATNKATYKPNTWCI
RCLWSTKPVETSNSFDVLKSEDAQGMDNLACEDLKPVSEEVVENPTIQKDVLECNVKTTEVVGDIIL
KPANNSLKITEEVGHTDLMAAYVDNSSLTIKKPNELSRVLGLKTLATHGLAAVNSVPWDTIANYAKP
FLNKVVSTTTNIVTRCLNRVCTNYMPYFFTLLLQLCTFTRSTNSRIKASMPTTIAKNTVKSVGKFCL
EASFNYLKSPNFSKLINIIIWFLLLSVCLGSLIYSTAALGVLMSNLGMPSYCTGYREGYLNSTNVTI
ATYCTGSIPCSVCLSGLDSLDTYPSLETIQITISSFKWDLTAFGLVAEWFLAYILFTRFFYVLGLAA
IMQLFFSYFAVHFISNSWLMWLIINLVQMAPISAMVRMYIFFASFYYVWKSYVHVVDGCNSSTCMMC
YKRNRATRVECTTIVNGVRRSFYVYANGGKGFCKLHNWNCVNCDTFCAGSTFISDEVARDLSLQFKR
PINPTDQSSYIVDSVTVKNGSIHLYFDKAGQKTYERHSLSHFVNLDNLRANNTKGSLPINVIVFDGK
SKCEESSAKSASVYYSQLMCQPILLLDQALVSDVGDSAEVAVKMFDAYVNTFSSTFNVPMEKLKTLV
ATAEAELAKNVSLDNVLSTFISAARQGFVDSDVETKDVVECLKLSHQSDIEVTGDSCNNYMLTYNKV
ENMTPRDLGACIDCSARHINAQVAKSHNIALIWNVKDFMSLSEQLRKQIRSAAKKNNLPFKLTCATT
RQVVNVVTTKIALKGGKIVNNWLKQLIKVTLVFLFVAAIFYLITPVHVMSKHTDFSSEIIGYKAIDG
GVTRDIASTDTCFANKHADFDTWFSQRGGSYTNDKACPLIAAVITREVGFVVPGLPGTILRTTNGDF
LHFLPRVFSAVGNICYTPSKLIEYTDFATSACVLAAECTIFKDASGKPVPYCYDTNVLEGSVAYESL
RPDTRYVLMDGSIIQFPNTYLEGSVRVVTTFDSEYCRHGTCERSEAGVCVSTSGRWVLNNDYYRSLP
GVFCGVDAVNLLTNMFTPLIQPIGALDISASIVAGGIVAIVVTCLAYYFMRFRRAFGEYSHVVAFNT
LLFLMSFTVLCLTPVYSFLPGVYSVIYLYLTFYLTNDVSFLAHIQWMVMFTPLVPFWITIAYIICIS
TKHFYWFFSNYLKRRVVFNGVSFSTFEEAALCTFLLNKEMYLKLRSDVLLPLTQYNRYLALYNKYKY
FSGAMDTTSYREAACCHLAKALNDFSNSGSDVLYQPPQTSITSAVLQSGFRKMAFPSGKVEGCMVQV
TCGTTTLNGLWLDDVVYCPRHVICTSEDMLNPNYEDLLIRKSNHNFLVQAGNVQLRVIGHSMQNCVL
KLKVDTANPKTPKYKFVRIQPGQTFSVLACYNGSPSGVYQCAMRPNFTIKGSFLNGSCGSVGFNIDY
DCVSFCYMHHMELPTGVHAGTDLEGNFYGPFVDRQTAQAAGTDTTITVNVLAWLYAAVINGDRWFLN
RFTTTLNDFNLVAMKYNYEPLTQDHVDILGPLSAQTGIAVLDMCASLKELLQNGMNGRTILGSALLE
DEFTPFDVVRQCSGVTFQSAVKRTIKGTHHWLLLTILTSLLVLVQSTQWSLFFFLYENAFLPFAMGI
IAMSAFAMMFVKHKHAFLCLFLLPSLATVAYFNMVYMPASWVMRIMTWLDMVDTSLSGFKLKDCVMY
ASAVVLLILMTARTVYDDGARRVWTLMNVLTLVYKVYYGNALDQAISMWALIISVTSNYSGVVTTVM
FLARGIVEMCVEYCPIFFITGNTLQCIMLVYCFLGYFCTCYFGLFCLLNRYFRLTLGVYDYLVSTQE
FRYMNSQGLLPPKNSIDAFKLNIKLLGVGGKPCIKVATVQSKMSDVKCTSVVLLSVLQQLRVESSSK
LWAQCVQLHNDILLAKDTTEAFEKMVSLLSVLLSMQGAVDINKLCEEMLDNRATLQAIASEFSSLPS
YAAFATAQEAYEQAVANGDSEVVLKKLKKSLNVAKSEFDRDAAMQRKLEKMADQAMTQMYKQARSED
KRAKVTSAMQTMLFTMLRKLDNDALNNIINNARDGCVPLNIIPLTTAAKLMVVIPDYNTYKNTCDGT
TFTYASALWEIQQVVDADSKIVQLSEISMDNSPNLAWPLIVTALRANSAVKLQNNELSPVALRQMSC
AAGTTQTACTDDNALAYYNTTKGGRFVLALLSDLQDLKWARFPKSDGTGTIYTELEPPCRFVTDTPK
GPKVKYLYFIKGLNNLNRGMVLGSLAATVRLQAGNATEVPANSTVLSFCAFAVDAAKAYKDYLASGG
QPITNCVKMLCTHTGTGQAITVTPEANMDQESFGGASCCLYCRCHIDHPNPKGFCDLKGKYVQIPTT
CANDPVGFTLKNTVCTVCGMWKGYGCSCDQLREPMLQSADAQSFLNRVCGVSAARLTPCGTGTSTDV
VYRAFDIYNDKVAGFAKFLKTNCCRFQEKDEDDNLIDSYFVVKRHTFSNYQHEETIYNLLKDCPAVA
KHDFFKFRIDGDMVPHISRQRLTKYTMADLVYALRHFDEGNCDTLKEILVTYNCCDDDYFNKKDWYD
FVENPDILRVYANLGERVRQALLKTVQFCDAMRNAGIVGVLTLDNQDLNGNWYDFGDFIQTTPGSGV
PVVDSYYSLLMPILTLTRALTAESHVDTDLTKPYIKWDLLKYDFTEERLKLFDRYFKYWDQTYHPNC
VNCLDDRCILHCANFNVLFSTVFPPTSFGPLVRKIFVDGVPFVVSTGYHFRELGVVHNQDVNLHSSR
LSFKELLVYAADPAMHAASGNLLLDKRTTCFSVAALTNNVAFQTVKPGNFNKDFYDFAVSKGFFKEG
SSVELKHFFFAQDGNAAISDYDYYRYNLPTMCDIRQLLFVVEVVDKYFDCYDGGCINANQVIVNNLD
KSAGFPFNKWGKARLYYDSMSYEDQDALFAYTKRNVIPTITQMNLKYAISAKNRARTVAGVSICSTM
TNRQFHQKLLKSIAATRGATVVIGTSKFYGGWHNMLKTVYSDVENPHLMGWDYPKCDRAMPNMLRIM
ASLVLARKHTTCCSLSHRFYRLANECAQVLSEMVMCGGSLYVKPGGTSSGDATTAYANSVFNICQAV
TANVNALLSTDGNKIADKYVRNLQHRLYECLYRNRDVDTDFVNEFYAYLRKHFSMMILSDDAVVCFN
STYASQGLVASIKNFKSVLYYQNNVFMSEAKCWTETDLTKGPHEFCSQHTMLVKQGDDYVYLPYPDP
SRILGAGCFVDDIVKTDGTLMIERFVSLAIDAYPLTKHPNQEYADVFHLYLQYIRKLHDELTGHMLD
MYSVMLTNDNTSRYWEPEFYEAMYTPHTVLQAVGACVLCNSQTSLRCGACIRRPFLCCKCCYDHVIS
TSHKLVLSVNPYVCNAPGCDVTDVTQLYLGGMSYYCKSHKPPISFPLCANGQVFGLYKNTCVGSDNV
TDFNAIATCDWTNAGDYILANTCTERLKLFAAETLKATEETFKLSYGIATVREVLSDRELHLSWEVG
KPRPPLNRNYVFTGYRVTKNSKVQIGEYTFEKGDYGDAVVYRGTTTYKLNVGDYFVLTSHTVMPLSA
PTLVPQEHYVRITGLYPTLNISDEFSSNVANYQKVGMQKYSTLQGPPGTGKSHFAIGLALYYPSARI
VYTACSHAAVDALCEKALKYLPIDKCSRIIPARARVECFDKFKVNSTLEQYVFCTVNALPETTADIV
VFDEISMATNYDLSVVNARLRAKHYVYIGDPAQLPAPRTLLTKGTLEPEYFNSVCRLMKTIGPDMFL
GTCRRCPAEIVDTVSALVYDNKLKAHKDKSAQCFKMFYKGVITHDVSSAINRPQIGVVREFLTRNPA
WRKAVFISPYNSQNAVASKILGLPTQTVDSSQGSEYDYVIFTQTTETAHSCNVNRFNVAITRAKVGI
LCIMSDRDLYDKLQFTSLEIPRRNVATLQAENVTGLFKDCSKVITGLHPTQAPTHLSVDTKFKTEGL
CVDIPGIPKDMTYRRLISMMGFKMNYQVNGYPNMFITREEAIRHVRAWIGFDVEGCHATREAVGTNL
PLQLGFSTGVNLVAVPTGYVDTPNNTDFSRVSAKPPPGDQFKHLIPLMYKGLPWNVVRIKIVQMLSD
TLKNLSDRVVFVLWAHGFELTSMKYFVKIGPERTCCLCDRRATCFSTASDTYACWHHSIGFDYVYNP
FMIDVQQWGFTGNLQSNHDLYCQVHGNAHVASCDAIMTRCLAVHECFVKRVDWTIEYPIIGDELKIN
AACRKVQHMVVKAALLADKFPVLHDIGNPKAIKCVPQADVEWKFYDAQPCSDKAYKIEELFYSYATH
SDKFTDGVCLFWNCNVDRYPANSIVCRFDTRVLSNLNLPGCDGGSLYVNKHAFHTPAFDKSAFVNLK
QLPFFYYSDSPCESHGKQVVSDIDYVPLKSATCITRCNLGGAVCRHHANEYRLYLDAYNMMISAGFS
LWVYKQFDTYNLWNTFTRLQSLENVAFNVVNKGHFDGQQGEVPVSIINNTVYTKVDGVDVELFENKT
TLPVNVAFELWAKRNIKPVPEVKILNNLGVDIAANTVIWDYKRDAPAHISTIGVCSMTDIAKKPTET
ICAPLTVFFDGRVDGQVDLFRNARNGVLITEGSVKGLQPSVGPKQASLNGVTLIGEAVKTQFNYYKK
VDGVVQQLPETYFTQSRNLQEFKPRSQMEIDFLELAMDEFIERYKLEGYAFEHIVYGDFSHSQLGGL
HLLIGLAKRFKESPFELEDFIPMDSTVKNYFITDAQTGSSKCVCSVIDLLLDDFVEIIKSQDLSVVS
KVVKVTIDYTEISFMLWCKDGHVETFYPKLQSSQAWQPGVAMPNLYKMQRMLLEKCDLQNYGDSATL
PKGIMMNVAKYTQLCQYLNTLTLAVPYNMRVIHFGAGSDKGVAPGTAVLRQWLPTGTLLVDSDLNDF
VSDADSTLIGDCATVHTANKWDLIISDMYDPKTKNVTKENDSKEGFFTYICGFIQQKLALGGSVAIK
ITEHSWNADLYKLMGHFAWWTAFVTNVNASSSEAFLIGCNYLGKPREQIDGYVMHANYIFWRNTNPI
QLSSYSLFDMSKFPLKLRGTAVMSLKEGQINDMILSLLSKGRLIIRENNRVVISSDVLVNN

In some embodiments, the antibodies described herein can be useful against SARS-CoV2 variants. For example, the variants can be: the UK variant B.1.1.7 (such as B.1.1.7 with S:E484K); the South African variant B.1.351; the California variant B.1.427; the California variant B.1.429; the Brazilian variant P.1; the Brazilian variant P.2; the New York variant B.1.526 (such as B.1.526 with S:E484K or B.1.526 with S:S477N); the New York variant B.1.526.1; the New York variant B.1.526.2, the amino acid mutations of each strain which can be accessed at https://outbreak.info/situation-reports #Lineage_Mutation, and is incorporated by reference in their entireties. For example, a variant of SARS-CoV2 has accession number YP_009724390.1. For example, a variant of SARS-CoV2 has accession number QHD43416.1.

The SARS-CoV2 variants can comprise, for instance, amino acid sequences having an identity to SEQ ID NO: 980 of at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%.

Antibody molecules obtained from humans relate to any of the classes IgG, IgM, IgA, IgE and IgD, which differ from one another by the nature of the heavy chain present in the molecule. Certain classes have subclasses as well, such as IgG₁, IgG₂, IgG₃, IgG₄. Furthermore, in humans, the light chain may be a kappa chain or a lambda chain. The term “antigen-binding site,” or “binding portion” refers to the part of the immunoglobulin molecule that participates in antigen binding. The antigen binding site is formed by amino acid residues of the N-terminal variable (“V”) regions of the heavy (“H”) and light (“L”) chains. Three highly divergent stretches within the V regions of the heavy and light chains, referred to as “hypervariable regions,” are interposed between more conserved flanking stretches known as “framework regions,” or “FRs”. Thus, the term “FR” can refer to amino acid sequences which are naturally found between, and adjacent to, hypervariable regions in immunoglobulins. In an antibody molecule, the three hypervariable regions of a light chain and the three hypervariable regions of a heavy chain are disposed relative to each other in three-dimensional space to form an antigen-binding surface. The antigen-binding surface is complementary to the three-dimensional surface of a bound antigen, and the three hypervariable regions of each of the heavy and light chains are referred to as “complementarity-determining regions,” or “CDRs.”

Minor variations in the amino acid sequences of proteins are provided by the antibodies described herein. The variations in the amino acid sequence can be when the sequence maintains at least about 30%, at least about 40%, at least about 50%, at least about 60%, at least about 70%, at least about 75%, at least about 80%, at least about 90%, at least about 95%, or at least about 99% amino acid identity to the SEQ ID NOS of the antibodies described herein. For example, conservative amino acid replacements can be utilized. Conservative replacements are those that take place within a family of amino acids that are related in their side chains, wherein the interchangeability of residues have similar side chains In certain embodiments, the antibodies described herein include variants. Such variants can include those having at least from about 46% to about 50% amino acid identity to the SEQ ID NOS of the antibodies described herein, or having at least from about 50.1% to about 55% amino acid identity to the SEQ ID NOS of the antibodies described herein, or having at least from about 55.10% to about 60% amino acid identity to the SEQ ID NOS of the antibodies described herein, or having from at least about 60.10% to about 65% amino acid identity to the SEQ ID NOS of the antibodies described herein, or having from about 65.10% to about 70% amino acid identity to the SEQ ID NOS of the antibodies described herein, or having at least from about 70.10% to about 75% amino acid identity to the SEQ ID NOS of the antibodies described herein, or having at least from about 75.10% to about 80% amino acid identity to the SEQ ID NOS of the antibodies described herein, or having at least from about 80.1% to about 85% amino acid identity to the SEQ ID NOS of the antibodies described herein, or having at least from about 85.10% to about 90% amino acid identity to the SEQ ID NOS of the antibodies described herein, or having at least from about 90.1% to about 95% amino acid identity to the SEQ ID NOS of the antibodies described herein, or having at least from about 95.1% to about 97% amino acid identity to the SEQ ID NOS of the antibodies described herein, or having at least from about 97.1% to about 99% amino acid identity to the SEQ ID NOS of the antibodies described herein.

The term “epitope” can include any protein determinant capable of specific binding to an immunoglobulin, a scFv, or a T-cell receptor. Epitopic determinants can consist of chemically active surface groupings of molecules such as amino acids or sugar side chains and usually have specific three-dimensional structural characteristics, as well as specific charge characteristics. For example, antibodies can be raised against N-terminal or C-terminal peptides of a polypeptide, for example the C terminal domain (CTD) of the spike protein SARS-CoV2. The spike protein of SARS-CoV2 has NCBI Reference Sequence: YP_009724390 (amino acid residues 1-1273; SEQ ID NO: 980) comprising sequence:

MFVFLVLLPLVSSQCVNLTTRTQLPPAYTNSFTRGVYYPDKVFRSSVLHSTQDLFLPFFSN
VTWFHAIHVSGTNGTKRFDNPVLPFNDGVYFASTEKSNIIRGWIFGTTLDSKTQSLLIVNN
ATNVVIKVCEFQFCNDPFLGVYYHKNNKSWMESEFRVYSSANNCTFEYVSQPFLMDLEGKQ
GNFKNLREFVFKNIDGYFKIYSKHTPINLVRDLPQGFSALEPLVDLPIGINITRFQTLLAL
HRSYLTPGDSSSGWTAGAAAYYVGYLQPRTFLLKYNENGTITDAVDCALDPLSETKCTLKS
FTVEKGIYQTSNFRVQPTESIVRFPNITNLCPFGEVFNATRFASVYAWNRKRISNCVADYS

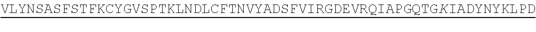

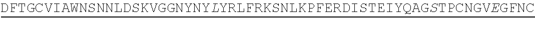

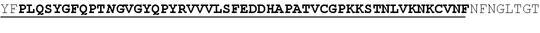

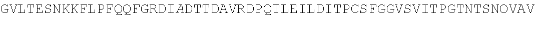

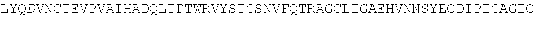

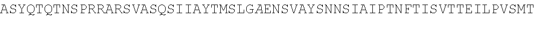

KTSVDCTMYICGDSTECSNLLLQYGSFCTQLNRALTGIAVEQDKNTQEVFAQVKQIYKTPP
IKDFGGFNFSQILPDPSKPSKRSFIEDLLFNKVTLADAGFIKQYGDCLGDIAARDLICAQK
FNGLTVLPPLLTDEMIAQYTSALLAGTITSGWTFGAGAALQIPFAMQMAYRFNGIGVTQNV
LYENQKLIANQFNSAIGKIQDSLSSTASALGKLQDVVNQNAQALNTLVKQLSSNFGAISSV
LNDILSRLDKVEAEVQIDRLITGRLQSLQTYVTQQLIRAAEIRASANLAATKMSECVLGQS
KRVDFCGKGYHLMSFPQSAPHGVVFLHVTYVPAQEKNFTTAPAICHDGKAHFPREGVFVSN
GTHWFVTQRNFYEPQIITTDNTFVSGNCDVVIGIVNNTVYDPLQPELDSFKEELDKYFKNH
TSPDVDLGDISGINASVVNIQKEIDRLNEVAKNLNESLIDLQELGKYEQYIKWPWYIWLGF
IAGLIAIVMVTIMLCCMTSCCSCLKGCCSCGSCCKFDEDDSEPVLKGVKLHYT

In some embodiments, the epitope comprises a region within amino acids 319-490 of the spike protein of SARS-CoV2 having NCBI Reference Sequence YP_009724390. In some embodiments, the epitope comprises a region within amino acids 319-541 of the spike protein of SARS-CoV2 having NCBI Reference Sequence YP_009724390. The exemplary, italicized shadowed amino acid residues of SEQ ID NO: 980 correspond to amino acid mutations found in SARS-CoV2 variant strains (e.g., K417N or K417T, L452R, S477N, E484K, N501Y, A570D, D614G, A701V).

The terms “immunological binding,” and “immunological binding properties” can refer to the non-covalent interactions of the type which occur between an immunoglobulin molecule and an antigen for which the immunoglobulin is specific. The strength, or affinity of immunological binding interactions can be expressed in terms of the dissociation constant (K_{d}) of the interaction, wherein a smaller K_{d} represents a greater affinity. Immunological binding properties of selected polypeptides can be quantified using methods well known in the art. One such method entails measuring the rates of antigen-binding site/antigen complex formation and dissociation, wherein those rates depend on the concentrations of the complex partners, the affinity of the interaction, and geometric parameters that equally influence the rate in both directions. Thus, both the “on rate constant” (Kₒₙ) and the “off rate constant” (K_{off}) can be determined by calculation of the concentrations and the actual rates of association and dissociation. (See Nature 361:186-87 (1993)). The ratio of K_{off}/Kₒₙ allows the cancellation of all parameters not related to affinity, and is equal to the dissociation constant K_{d}. (See, generally, Davies et al. (1990) Annual Rev Biochem 59:439-473). An antibody of the present invention can specifically bind to a SARS-CoV2 epitope when the equilibrium binding constant (K_{D}) is ≤1 μM, ≤10 μM, ≤10 nM, ≤10 pM, or ≤100 pM to about 1 pM, as measured by assays such as radioligand binding assays or similar assays known to those skilled in the art, such as BIAcore or Octet (BLI). For example, in some embodiments, the K_{D} is between about 1E-12 M and a K_{D} about 1E-11 M. In some embodiments, the K_{D} is between about 1E-11 M and a K_{D} about 1E-10 M. In some embodiments, the K_{D} is between about 1E-10 M and a K_{D} about 1E-9 M. In some embodiments, the K_{D} is between about 1E-9 M and a K_{D} about 1E-8 M. In some embodiments, the K_{D} is between about 1E-8 M and a K_{D} about 1E-7 M. In some embodiments, the K_{D} is between about 1E-7 M and a K_{D} about 1E-6 M. For example, in some embodiments, the K_{D} is about 1E-12 M while in other embodiments the K_{D} is about 1E-11 M. In some embodiments, the K_{D} is about 1E-10 M while in other embodiments the K_{D} is about 1E-9 M. In some embodiments, the K_{D} is about 1E-8 M while in other embodiments the K_{D} is about 1E-7 M. In some embodiments, the K_{D} is about 1E-6 M while in other embodiments the K_{D} is about 1E-5 M. In some embodiments, for example, the K_{D} is about 3 E-11 M, while in other embodiments the K_{D} is about 3E-12 M. In some embodiments, the K_{D} is about 6E-11 M. “Specifically binds” or “has specificity to,” can refer to an antibody that binds to an epitope via its antigen-binding domain, and that the binding entails some complementarity between the antigen-binding domain and the epitope. For example, an antibody is said to “specifically bind” to an epitope when it binds to that epitope, via its antigen-binding domain more readily than it would bind to a random, unrelated epitope.

For example, the SARS-CoV2 antibody can be monovalent or bivalent, and comprises a single or double chain. Functionally, the binding affinity of the SARS-CoV2 antibody is within the range of 10⁻⁵M to 10⁻¹² M. For example, the binding affinity of the SARS-CoV2 antibody is from 10⁻⁶ M to 10⁻¹² M, from 10⁻⁷ M to 10⁻¹² M, from 10⁻⁸ M to 10⁻¹² M, from 10⁻⁹ M to 10⁻¹² M, from 10⁻⁵ M to 10⁻¹¹ M, from 10⁻⁶ M to 10⁻¹¹ M, from 10⁻⁷ M to 10⁻¹¹ M, from 10⁻⁸ M to 10⁻¹¹ M, from 10⁻⁹ M to 10⁻¹¹ M, from 10⁻¹⁰ M to 10⁻¹¹ M, from 10⁻⁵ M to 10⁻¹⁰M, from 10⁻⁶ M to 10⁻¹⁰ M, from 10⁻⁷ M to 10⁻¹⁰ M, from 10⁻⁸ M to 10⁻¹⁰M, from 10⁻⁹ M to 10⁻¹⁰ M, from 10⁻⁵ M to 10⁻⁹ M, from 10⁻⁶ M to 10⁻⁹M, from 10⁻⁷ M to 10⁻⁹ M, from 10⁻⁸ M to 10⁻⁹ M, from 10⁻⁵ M to 10⁻⁸ M, from 10⁻⁶ M to 10⁻⁸ M, from 10⁻⁷ M to 10⁻⁸ M, from 10⁻⁵ M to 10⁻⁷ M, from 10⁻⁶ M to 10⁻⁷ M, or from 10⁻⁵ M to 10⁻⁶ M.

A SARS-CoV2 protein or a derivative, fragment, analog, homolog or ortholog thereof, can be utilized as an immunogen in the generation of antibodies that immunospecifically bind these protein components.

Those skilled in the art will recognize that it is possible to determine, without undue experimentation, if a human monoclonal antibody has the same specificity as a human monoclonal antibody of the invention by ascertaining whether the former prevents the latter from binding to SARS-CoV2. If the human monoclonal antibody being tested competes with the human monoclonal antibody of the invention, as shown by a decrease in binding by the human monoclonal antibody of the invention, then it is likely that the two monoclonal antibodies bind to the same, or to a closely related, epitope.

Another way to determine whether a human monoclonal antibody has the specificity of a human monoclonal antibody of the invention is to pre-incubate the human monoclonal antibody of the invention with the SARS-CoV2 with which it is normally reactive, and then add the human monoclonal antibody being tested to determine if the human monoclonal antibody being tested is inhibited in its ability to bind SARS-CoV2. If the human monoclonal antibody being tested is inhibited then, in all likelihood, it has the same, or functionally equivalent, epitopic specificity as the monoclonal antibody of the invention.

Various procedures known within the art may be used for the production of polyclonal or monoclonal antibodies directed against a protein of the invention, or against derivatives, fragments, analogs homologs or orthologs thereof (See, for example, Antibodies: A Laboratory Manual, Harlow E, and Lane D, 1988, Cold Spring Harbor Laboratory Press, Cold Spring Harbor, NY, incorporated herein by reference).

Antibodies can be purified by well-known techniques, such as affinity chromatography using protein A or protein G, which provide primarily the IgG fraction of immune serum. Subsequently, or alternatively, the specific antigen which is the target of the immunoglobulin sought, or an epitope thereof, may be immobilized on a column to purify the immune specific antibody by immunoaffinity chromatography. Purification of immunoglobulins is discussed, for example, by D. Wilkinson (The Scientist, published by The Scientist, Inc., Philadelphia PA, Vol. 14, No. 8 (Apr. 17, 2000), pp. 25-28).

The term “monoclonal antibody” or “MAb” or “monoclonal antibody composition”, as used herein, can refer to a population of antibody molecules that contain only one molecular species of antibody molecule consisting of a unique light chain gene product and a unique heavy chain gene product. For example, the complementarity determining regions (CDRs) of the monoclonal antibody are identical in all the molecules of the population. MAbs contain an antigen binding site capable of immunoreacting with an epitope of the antigen characterized by a unique binding affinity for it.

Monoclonal antibodies can be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes can be immunized in vitro.

The immunizing agent can include the protein antigen, a fragment thereof or a fusion protein thereof. For example, either peripheral blood lymphocytes are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, Monoclonal Antibodies: Principles and Practice, Academic Press, (1986) pp. 59-103). Immortalized cell lines are usually transformed mammalian cells, such as myeloma cells of rodent, bovine and human origin. Usually, rat or mouse myeloma cell lines are employed. The hybridoma cells can be cultured in a suitable culture medium that contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Immortalized cell lines include those that fuse efficiently, support stable high level expression of antibody by the selected antibody-producing cells, and are sensitive to a medium such as HAT medium. Immortalized cell lines can also include murine myeloma lines, which can be obtained, for instance, from the Salk Institute Cell Distribution Center, San Diego, California and the American Type Culture Collection, Manassas, Virginia. Human myeloma and mouse-human heteromyeloma cell lines also have been described for the production of human monoclonal antibodies. (See Kozbor, J. Immunol., 133:3001 (1984); Brodeur et al., Monoclonal Antibody Production Techniques and Applications, Marcel Dekker, Inc., New York, (1987) pp. 51-63)).

The culture medium in which the hybridoma cells are cultured can then be assayed for the presence of monoclonal antibodies directed against the antigen. The binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980). Moreover, in therapeutic applications of monoclonal antibodies, it is important to identify antibodies having a high degree of specificity and a high binding affinity for the target antigen.

After the desired hybridoma cells are identified, the clones can be subcloned by limiting dilution procedures and grown by standard methods. (See Goding, Monoclonal Antibodies: Principles and Practice, Academic Press, (1986) pp. 59-103). Suitable culture media for this purpose include, for example, Dulbecco's Modified Eagle's Medium and RPMI-1640 medium. Alternatively, the hybridoma cells can be grown in vivo as ascites in a mammal.

The monoclonal antibodies secreted by the subclones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

Monoclonal antibodies can also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences (see U.S. Pat. No. 4,816,567; Morrison, Nature 368, 812-13 (1994)) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

Fully human antibodies are antibody molecules in which the entire sequence of both the light chain and the heavy chain, including the CDRs, arise from human genes. Such antibodies are termed “human antibodies”, or “fully human antibodies” herein. Human monoclonal antibodies can be prepared by using trioma technique; the human B-cell hybridoma technique (see Kozbor, et al., 1983 Immunol Today 4: 72); and the EBV hybridoma technique to produce human monoclonal antibodies (see Cole, et al., 1985 In: MONOCLONAL ANTIBODIES AND CANCER THERAPY, Alan R. Liss, Inc., pp. 77-96). Human monoclonal antibodies may be utilized and may be produced by using human hybridomas (see Cote, et al., 1983. Proc Natl Acad Sci USA 80: 2026-2030) or by transforming human B-cells with Epstein Barr Virus in vitro (see Cole, et al., 1985 In: MONOCLONAL ANTIBODIES AND CANCER THERAPY, Alan R. Liss, Inc., pp. 77-96).

“Humanized antibodies” can be antibodies from a non-human species (such as mouse), whose amino acid sequences (for example, in the CDR regions) have been modified to increase their similarity to antibody variants produced in humans. Antibodies can be humanized by methods known in the art, such as CDR-grafting. See also, Safdari et al., (2013) Biotechnol Genet Eng Rev.; 29:175-86. In addition, humanized antibodies can be produced in transgenic plants, as an inexpensive production alternative to existing mammalian systems. For example, the transgenic plant may be a tobacco plant, i.e., Nicotiana benthamiana, and Nicotiana tabaccum. The antibodies are purified from the plant leaves. Stable transformation of the plants can be achieved through the use of Agrobacterium tumefaciens or particle bombardment. For example, nucleic acid expression vectors containing at least the heavy and light chain sequences are expressed in bacterial cultures, i.e., A. tumefaciens strain BLA4404, via transformation. Infiltration of the plants can be accomplished via injection. Soluble leaf extracts can be prepared by grinding leaf tissue in a mortar and by centrifugation. Isolation and purification of the antibodies can be readily be performed by many of the methods known to the skilled artisan in the art. Other methods for antibody production in plants are described in, for example, Fischer et al., Vaccine, 2003, 21:820-5; and Ko et al, Current Topics in Microbiology and Immunology, Vol. 332, 2009, pp. 55-78. As such, the present invention further provides any cell or plant comprising a vector that encodes the antibody of the present invention, or produces the antibody of the present invention.

In addition, human antibodies can also be produced using additional techniques, including phage display libraries. (See Hoogenboom and Winter, J. Mol. Biol., 227:381 (1991); Marks et al., J. Mol. Biol., 222:581 (1991)). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in Marks et al., Bio/Technology 10, 779-783 (1992); Lonberg et al., Nature 368 856-859 (1994); Morrison, Nature 368, 812-13 (1994); Fishwild et al, Nature Biotechnology 14, 845-51 (1996); Neuberger, Nature Biotechnology 14, 826 (1996); and Lonberg and Huszar, Intern. Rev. Immunol. 73 65-93 (1995).

Human antibodies can additionally be produced using transgenic nonhuman animals which are modified so as to produce fully human antibodies rather than the animal's endogenous antibodies in response to challenge by an antigen. (See PCT publication WO94/02602 and U.S. Pat. No. 6,673,986). The endogenous genes encoding the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal which provides all the desired modifications is then obtained as progeny by crossbreeding intermediate transgenic animals containing fewer than the full complement of the modifications. The embodiment of such a nonhuman animal is a mouse, and is termed the Xenomouse™ as disclosed in PCT publications WO 96/33735 and WO 96/34096. This animal produces B cells which secrete fully human immunoglobulins. The antibodies can be obtained directly from the animal after immunization with an immunogen of interest, as, for example, a preparation of a polyclonal antibody, or alternatively from immortalized B cells derived from the animal, such as hybridomas producing monoclonal antibodies. Additionally, the genes encoding the immunoglobulins with human variable regions can be recovered and expressed to obtain the antibodies directly, or can be further modified to obtain analogs of antibodies such as, for example, single chain Fv (scFv) molecules.

Thus, using such a technique, therapeutically useful IgG, IgA, IgM and IgE antibodies can be produced. For an overview of this technology for producing human antibodies, see Lonberg and Huszar Int. Rev. Immunol. 73:65-93 (1995). For a detailed discussion of this technology for producing human antibodies and human monoclonal antibodies and protocols for producing such antibodies, see, e.g., PCT publications WO 98/24893; WO 96/34096; WO 96/33735; U.S. Pat. Nos. 5,413,923; 5,625,126; 5,633,425; 5,569,825; 5,661,016; 5,545,806; 5,814,318; and 5,939,598, which are incorporated by reference herein in their entirety. In addition, companies such as Creative BioLabs (Shirley, NY) can be engaged to provide human antibodies directed against a selected antigen using technology similar to that described herein.

An example of a method of producing a nonhuman host, exemplified as a mouse, lacking expression of an endogenous immunoglobulin heavy chain is disclosed in U.S. Pat. No. 5,939,598. It can be obtained by a method, which includes deleting the J segment genes from at least one endogenous heavy chain locus in an embryonic stem cell to prevent rearrangement of the locus and to prevent formation of a transcript of a rearranged immunoglobulin heavy chain locus, the deletion being effected by a targeting vector containing a gene encoding a selectable marker; and producing from the embryonic stem cell a transgenic mouse whose somatic and germ cells contain the gene encoding the selectable marker.

One method for producing an antibody of interest, such as a human antibody, is disclosed in U.S. Pat. No. 5,916,771. This method includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

In a further improvement on this procedure, a method for identifying a clinically relevant epitope on an immunogen and a correlative method for selecting an antibody that binds immunospecifically to the relevant epitope with high affinity, are disclosed in PCT publication WO 99/53049.

The antibody can be expressed by a vector containing a DNA segment encoding the single chain antibody described herein.

These vectors can include liposomes, naked DNA, adjuvant-assisted DNA, gene gun, catheters, etc. Vectors can include chemical conjugates such as described in WO 93/64701, which has targeting moiety (e.g. a ligand to a cellular surface receptor), and a nucleic acid binding moiety (e.g. polylysine), viral vectors (e.g. a DNA or RNA viral vector), fusion proteins such as described in PCT/US 95/02140 (WO 95/22618) which is a fusion protein containing a target moiety (e.g. an antibody specific for a target cell) and a nucleic acid binding moiety (e.g. a protamine), plasmids, phage, etc. The vectors can be chromosomal, non-chromosomal or synthetic. Retroviral vectors can also be used, and include moloney murine leukemia viruses.

DNA viral vectors can also be used, and include pox vectors such as orthopox or avipox vectors, herpesvirus vectors such as a herpes simplex I virus (HSV) vector (see Geller, A. I. et al., J. Neurochem, 64:487 (1995); Lim, F., et al., in DNA Cloning: Mammalian Systems, D. Glover, Ed. (Oxford Univ. Press, Oxford England) (1995); Geller, A. I. et al., Proc Natl. Acad. Sci.: U.S.A. 90:7603 (1993); Geller, A. I., et al., Proc Natl. Acad. Sci USA 87:1149 (1990), Adenovirus Vectors (see LeGal LaSalle et al., Science, 259:988 (1993); Davidson, et al., Nat. Genet 3:219 (1993); Yang, et al., J. Virol. 69:2004 (1995) and Adeno-associated Virus Vectors (see Kaplitt, M. G. et al., Nat. Genet. 8:148 (1994).

Pox viral vectors introduce the gene into the cell's cytoplasm. Avipox virus vectors result in only a short-term expression of the nucleic acid. Adenovirus vectors, adeno-associated virus vectors and herpes simplex virus (HSV) vectors are useful for introducing the nucleic acid into neural cells. The adenovirus vector results in a shorter-term expression (about 2 months) than adeno-associated virus (about 4 months), which in turn is shorter than HSV vectors. The vector chosen will depend upon the target cell and the condition being treated. The introduction can be by standard techniques, e.g. infection, transfection, transduction or transformation. Examples of modes of gene transfer include e.g., naked DNA, CaPO₄ precipitation, DEAE dextran, electroporation, protoplast fusion, lipofection, cell microinjection, and viral vectors.

The vector can be employed to target essentially any desired target cell. For example, stereotaxic injection can be used to direct the vectors (e.g. adenovirus, HSV) to a desired location. Additionally, the particles can be delivered by intracerebroventricular (icv) infusion using a minipump infusion system, such as a SynchroMed Infusion System. A method based on bulk flow, termed convection, has also proven effective at delivering large molecules to extended areas of the brain and may be useful in delivering the vector to the target cell. (See Bobo et al., Proc. Natl. Acad. Sci. USA 91:2076-2080 (1994); Morrison et al., Am. J. Physiol. 266:292-305 (1994)). Other methods that can be used include catheters, intravenous, parenteral, intraperitoneal and subcutaneous injection, and oral or other known routes of administration.

These vectors can be used to express large quantities of antibodies that can be used in a variety of ways. For example, to detect the presence of SARS-CoV2 in a sample. The antibody can also be used to try to bind to and disrupt SARS-CoV2.

In an embodiment, the antibodies of the present invention are full-length antibodies, containing an Fc region similar to wild-type Fc regions that bind to Fc receptors.

Heteroconjugate antibodies are also within the scope of the present invention. Heteroconjugate antibodies are composed of two covalently joined antibodies. It is contemplated that the antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

In embodiments, the antibody of the invention can be modified with respect to effector function, so as to enhance, e.g., the effectiveness of the antibody in neutralizing or preventing viral infection. For example, cysteine residue(s) can be introduced into the Fc region, thereby allowing interchain disulfide bond formation in this region. The homodimeric antibody thus generated can have improved internalization capability and/or increased complement-mediated cell killing and antibody-dependent cellular cytotoxicity (ADCC). (See Caron et al., J. Exp Med., 176: 1191-1195 (1992) and Shopes, J. Immunol., 148: 2918-2922 (1992)). Alternatively, an antibody can be engineered that has dual Fc regions and can thereby have enhanced complement lysis and ADCC capabilities. (See Stevenson et al., Anti-Cancer Drug Design, 3: 219-230 (1989)). In one embodiment, the antibody of the present invention has modifications of the Fc region, such that the Fc region does not bind to the Fc receptors. For example, the Fc receptor is Fcγ receptor. Antibodies with modification of the Fc region such that the Fc region does not bind to Fcγ, but still binds to neonatal Fc receptor are useful as described herein.

In certain embodiments, an antibody of the invention can comprise an Fc variant comprising an amino acid substitution which alters the antigen-independent effector functions of the antibody, in particular the circulating half-life of the antibody. Such antibodies exhibit either increased or decreased binding to FcRn when compared to antibodies lacking these substitutions, therefore, have an increased or decreased half-life in serum, respectively. Fc variants with improved affinity for FcRn are anticipated to have longer serum half-lives, and such molecules have useful applications in methods of treating mammals where long half-life of the administered antibody is desired, e.g., to treat a chronic disease or disorder. In contrast, Fc variants with decreased FcRn binding affinity are expected to have shorter halt-lives, and such molecules are also useful, for example, for administration to a mammal where a shortened circulation time can be advantageous, e.g., for in vivo diagnostic imaging or in situations where the starting antibody has toxic side effects when present in the circulation for prolonged periods. Fc variants with decreased FcRn binding affinity are also less likely to cross the placenta and, thus, are also useful in the treatment of diseases or disorders in pregnant women. In addition, other applications in which reduced FcRn binding affinity can be desired include those applications in which localization to the brain, kidney, and/or liver is desired. In one embodiment, the Fc variant-containing antibodies can exhibit reduced transport across the epithelium of kidney glomeruli from the vasculature. In another embodiment, the Fc variant-containing antibodies can exhibit reduced transport across the blood brain barrier (BBB) from the brain, into the vascular space. In one embodiment, an antibody with altered FcRn binding comprises an Fc domain having one or more amino acid substitutions within the “FcRn binding loop” of an Fc domain. The FcRn binding loop is comprised of amino acid residues 280-299 (according to EU numbering). Exemplary amino acid substitutions with altered FcRn binding activity are disclosed in PCT Publication No. WO05/047327 which is incorporated by reference herein. In certain exemplary embodiments, the antibodies, or fragments thereof, of the invention comprise an Fc domain having one or more of the following substitutions: V284E, H285E, N286D, K290E and S304D (EU numbering).

In some embodiments, mutations are introduced to the constant regions of the mAb such that the antibody dependent cell-mediated cytotoxicity (ADCC) activity of the mAb is altered. For example, the mutation is a LALA mutation in the CH2 domain. In one embodiment, the antibody (e.g., a human mAb, or a bispecific Ab) contains mutations on one scFv unit of the heterodimeric mAb, which reduces the ADCC activity. In another embodiment, the mAb contains mutations on both chains of the heterodimeric mAb, which completely ablates the ADCC activity. For example, the mutations introduced into one or both scFv units of the mAb are LALA mutations in the CH2 domain. These mAbs with variable ADCC activity can be optimized such that the mAbs exhibits maximal selective killing towards cells that express one antigen that is recognized by the mAb, however exhibits minimal killing towards the second antigen that is recognized by the mAb.

In other embodiments, antibodies of the invention for use in the diagnostic and treatment methods described herein have a constant region, e.g., an IgG₁ or IgG₄ heavy chain constant region, which can be altered to reduce or eliminate glycosylation. For example, an antibody of the invention can also comprise an Fc variant comprising an amino acid substitution which alters the glycosylation of the antibody. For example, the Fc variant can have reduced glycosylation (e.g., N- or O-linked glycosylation). In some embodiments, the Fc variant comprises reduced glycosylation of the N-linked glycan normally found at amino acid position 297 (EU numbering). In another embodiment, the antibody has an amino acid substitution near or within a glycosylation motif, for example, an N-linked glycosylation motif that contains the amino acid sequence NXT or NXS. In one embodiment, the antibody comprises an Fc variant with an amino acid substitution at amino acid position 228 or 299 (EU numbering). In more particular embodiments, the antibody comprises an IgG1 or IgG4 constant region comprising an S228P and a T299A mutation (EU numbering).

Exemplary amino acid substitutions which confer reduced or altered glycosylation are described in PCT Publication No, WO05/018572, which is incorporated by reference herein in its entirety. In some embodiments, the antibodies of the invention, or fragments thereof, are modified to eliminate glycosylation. Such antibodies, or fragments thereof, can be referred to as “agly” antibodies, or fragments thereof, (e.g. “agly” antibodies). While not wishing to be bound by theory “agly” antibodies, or fragments thereof, can have an improved safety and stability profile in vivo. Exemplary agly antibodies, or fragments thereof, comprise an aglycosylated Fc region of an IgG₄ antibody which is devoid of Fc-effector function thereby eliminating the potential for Fc mediated toxicity to the normal vital tissues. In yet other embodiments, antibodies of the invention, or fragments thereof, comprise an altered glycan. For example, the antibody can have a reduced number of fucose residues on an N-glycan at Asn297 of the Fc region, i.e., is afucosylated. In another embodiment, the antibody can have an altered number of sialic acid residues on the N-glycan at Asn297 of the Fc region.

The invention also pertains to immunoconjugates comprising an antibody conjugated to a cytotoxic agent such as a toxin (e.g., an enzymatically active toxin of bacterial, fungal, plant, or animal origin, or fragments thereof), or a radioactive isotope (i.e., a radioconjugate).

Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes. A variety of radionuclides are available for the production of radioconjugated antibodies. Examples include ²¹²Bi, ¹³¹I, ¹³¹In, ⁹⁰Y, and ¹⁸⁶Re.

Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. (See WO94/11026).

Those of ordinary skill in the art will recognize that a large variety of moieties can be coupled to the resultant antibodies or to other molecules of the invention. (See, for example, “Conjugate Vaccines”, Contributions to Microbiology and Immunology, J. M. Cruse and R. E. Lewis, Jr (eds), Carger Press, New York, (1989), the entire contents of which are incorporated herein by reference).

Coupling may be accomplished by any chemical reaction that will bind the two molecules so long as the antibody and the other moiety retain their respective activities. This linkage can include many chemical mechanisms, for instance covalent binding, affinity binding, intercalation, coordinate binding and complexation. The binding is, however, covalent binding. Covalent binding can be achieved either by direct condensation of existing side chains or by the incorporation of external bridging molecules. Many bivalent or polyvalent linking agents are useful in coupling protein molecules, such as the antibodies of the present invention, to other molecules. For example, representative coupling agents can include organic compounds such as thioesters, carbodiimides, succinimide esters, diisocyanates, glutaraldehyde, diazobenzenes and hexamethylene diamines. This listing is not intended to be exhaustive of the various classes of coupling agents known in the art but, rather, is exemplary of the more common coupling agents. (See Killen and Lindstrom, Jour. Immun. 133:1335-2549 (1984); Jansen et al., Immunological Reviews 62:185-216 (1982); and Vitetta et al., Science 238:1098 (1987)). Examples of linkers are described in the literature. (See, for example, Ramakrishnan, S. et al., Cancer Res. 44:201-208 (1984) describing use of MBS (M-maleimidobenzoyl-N-hydroxysuccinimide ester). See also, U.S. Pat. No. 5,030,719, describing use of halogenated acetyl hydrazide derivative coupled to an antibody by way of an oligopeptide linker. Useful linkers include: (i) EDC (1-ethyl-3-(3-dimethylamino-propyl) carbodiimide hydrochloride; (ii) SMPT (4-succinimidyloxycarbonyl-alpha-methyl-alpha-(2-pridyl-dithio)-toluene (Pierce Chem. Co., Cat. (21558G); (iii) SPDP (succinimidyl-6 [3-(2-pyridyldithio) propionamido]hexanoate (Pierce Chem. Co., Cat #21651G); (iv) Sulfo-LC-SPDP (sulfosuccinimidyl 6 [3-(2-pyridyldithio)-propianamide] hexanoate (Pierce Chem. Co. Cat. #2165-G); and (v) sulfo-NHS (N-hydroxysulfo-succinimide: Pierce Chem. Co., Cat. #24510) conjugated to EDC.

The linkers described above contain components that have different attributes, thus leading to conjugates with differing physio-chemical properties. For example, sulfo-NHS esters of alkyl carboxylates are more stable than sulfo-NHS esters of aromatic carboxylates. NHS-ester containing linkers are less soluble than sulfo-NHS esters. Further, the linker SMPT contains a sterically hindered disulfide bond, and can form conjugates with increased stability. Disulfide linkages, are in general, less stable than other linkages because the disulfide linkage is cleaved in vitro, resulting in less conjugate available. Sulfo-NHS, for example, can enhance the stability of carbodimide couplings. Carbodimide couplings (such as EDC) when used in conjunction with sulfo-NHS, forms esters that are more resistant to hydrolysis than the carbodimide coupling reaction alone.

The antibodies disclosed herein can also be formulated as immunoliposomes. Liposomes containing the antibody are prepared by methods known in the art, such as described in Epstein et al., Proc. Natl. Acad. Sci. USA, 82: 3688 (1985); Hwang et al., Proc. Natl Acad. Sci. USA, 77: 4030 (1980); and U.S. Pat. Nos. 4,485,045 and 4,544,545. Liposomes with enhanced circulation time are disclosed in U.S. Pat. No. 5,013,556.

Non-limiting example of useful liposomes can be generated by the reverse-phase evaporation method with a lipid composition comprising phosphatidylcholine, cholesterol, and PEG-derivatized phosphatidylethanolamine (PEG-PE). Liposomes are extruded through filters of defined pore size to yield liposomes with the desired diameter. Fab′ fragments of the antibody of the present invention can be conjugated to the liposomes as described in Martin et al., J. Biol. Chem., 257: 286-288 (1982) via a disulfide-interchange reaction.

The generation of neutralization escape mutants can be a helpful tool for identifying residues critical for neutralization and for investigating virus evolution under immune pressure (37, 49). Like other RNA viruses, CoVs have high mutation rates, especially during cross-species transmission, which is important for virus adaptation to new host receptors (5, 6, 50). Immune pressure is another force selecting virus mutation (37, 49). In addition, in view of current recommendations by the International Severe Acute Respiratory and Emerging Infection Consortium (ISARIC) and the increasing recognition that human Abs may have a role in the management of infectious diseases, the therapeutic potential of these nAbs should be considered for the prophylaxis and treatment of SARS (53). While escape from neutralization is a concern with therapeutic Abs, our study provides reagents and a strategy to mitigate this potential problem.

In another embodiment, the antibodies that neutralize infection by Severe Acute Respiratory Syndrome-associated coronavirus (SARS-CoV2) can be belong to various kinds of antibody classes and isotypes. For example, the neutralizing antibodies can be IgG₁, IgG₂, IgG₃ and/or IgG₄ isotype antibodies.

In another embodiment, the neutralizing antibodies can also contain LALA mutations in the Fc region. The LALA double mutants are characterized by the L234A L235A amino acid substitutions.

The humanized antibodies described herein can be produced in mammalian expression systems, such as hybridomas. The humanized antibodies described herein may also be produced by non-mammalian expression systems, for example, by transgenic plants. For example, the antibodies described herein are produced in transformed tobacco plants (N. benthamiana and N. tabaccum).

Use of Antibodies Against SARS-CoV2

Methods for the screening of antibodies that possess the desired specificity include, but are not limited to, enzyme linked immunosorbent assay (ELISA) and other immunologically mediated techniques known within the art.

Antibodies directed against a SARS-CoV2 protein disclosed herein can be useful in treatment of chronic infections, diseases, or medical conditions associated with COVID-19._Antibodies directed against a SARS-CoV2 protein, such as the spike protein, can be used in methods known within the art relating to the localization and/or quantitation of SARS-CoV2 (e.g., for use in measuring levels of the SARS-CoV2 protein within appropriate physiological samples, for use in diagnostic methods, for use in imaging the protein, and the like). In a given embodiment, antibodies specific to a SARS-CoV2, or derivative, fragment, analog or homolog thereof, that contain the antibody derived antigen binding domain, are utilized as pharmacologically active compounds (referred to hereinafter as “Therapeutics”).

An antibody specific for a SARS-CoV2 protein can be used to isolate a SARS-CoV2 polypeptide by standard techniques, such as immunoaffinity, chromatography or immunoprecipitation. Antibodies directed against a SARS-CoV2 protein (or a fragment thereof) can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

Antibodies of the invention, including polyclonal, monoclonal, humanized and fully human antibodies, can be used as therapeutic agents. Such agents will generally be employed to treat or prevent a SARS-CoV2 related disease or pathology in a subject. An antibody preparation, for example, one having high specificity and high affinity for its target antigen, is administered to the subject and will generally have an effect due to its binding with the target. Administration of the antibody may abrogate or inhibit or interfere with the internalization of the virus into a cell. In this case, the antibody binds to the target and prevents SARS-CoV2 binding the ACE2 receptor.

A therapeutically effective amount of an antibody of the invention relates generally to the amount needed to achieve a therapeutic objective. As noted above, this can be a binding interaction between the antibody and its target antigen that, in certain cases, interferes with the functioning of the target. The amount required to be administered will furthermore depend on the binding affinity of the antibody for its specific antigen, and will also depend on the rate at which an administered antibody is depleted from the free volume other subject to which it is administered. Common ranges for therapeutically effective dosing of an antibody or antibody fragment of the invention may be, by way of nonlimiting example, from about 0.1 mg/kg body weight to about 50 mg/kg body weight. Common dosing frequencies may range, for example, from twice daily to once a week.

Antibodies specifically binding a SARS-CoV2 protein or a fragment thereof of the invention, as well as other molecules identified by the screening assays disclosed herein, can be administered for the treatment of SARS-CoV2-related disorders in the form of pharmaceutical compositions. Principles and considerations involved in preparing such compositions, as well as guidance in the choice of components are provided, for example, in Remington: The Science And Practice Of Pharmacy 19th ed. (Alfonso R. Gennaro, et al., editors) Mack Pub. Co., Easton, Pa., 1995; Drug Absorption Enhancement: Concepts, Possibilities, Limitations, And Trends, Harwood Academic Publishers, Langhorne, Pa., 1994; and Peptide And Protein Drug Delivery (Advances In Parenteral Sciences, Vol. 4), 1991, M. Dekker, New York.

Embodiments of the present invention can comprise antibody fragments, such as antibody fragments lacking an Fc region. Peptide molecules can be designed that retain the ability to bind the target protein sequence. Such peptides can be synthesized chemically and/or produced by recombinant DNA technology. (See, e.g., Marasco et al., Proc. Natl. Acad. Sci. USA, 90: 7889-7893 (1993)). The formulation can also contain more than one active compound as necessary for the indication being treated, such as those with complementary activities that do not adversely affect each other. Alternatively, or in addition, the composition can comprise an agent that enhances its function, such as, for example, a cytotoxic agent, cytokine, chemotherapeutic agent, or growth-inhibitory agent. Such molecules are suitably present in combination in amounts that are effective for the purpose intended.

The active ingredients can also be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles, and nanocapsules) or in macroemulsions.

The formulations to be used for in vivo administration must be sterile. This is readily accomplished by filtration through sterile filtration membranes.

Sustained-release preparations can be prepared. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid allows for release of molecules for over 100 days, certain hydrogels release proteins for shorter time periods.

An antibody according to the invention can be used as an agent for detecting the presence of a SARS-CoV2 (or a protein or a protein fragment thereof) in a sample. In embodiments, the antibody contains a detectable label. Antibodies can be polyclonal, or for example, monoclonal. In embodiments, the antibody is an intact antibody. The term “labeled”, with regard to the probe or antibody, can encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” can include tissues, cells and biological fluids isolated from a subject, as well as tissues, cells and fluids present within a subject. Included within the usage of the term “biological sample”, therefore, is blood and a fraction or component of blood including blood serum, blood plasma, or lymph. That is, the detection method of the invention can be used to detect an analyte mRNA, protein, or genomic DNA in a biological sample in vitro as well as in vivo. For example, in vitro techniques for detection of an analyte mRNA include Northern hybridizations and in situ hybridizations. In vitro techniques for detection of an analyte protein include enzyme linked immunosorbent assays (ELISAs), Western blots, immunoprecipitations, and immunofluorescence. In vitro techniques for detection of an analyte genomic DNA include Southern hybridizations. Procedures for conducting immunoassays are described, for example in “ELISA: Theory and Practice: Methods in Molecular Biology”, Vol. 42, J. R. Crowther (Ed.) Human Press, Totowa, N J, 1995; “Immunoassay”, E. Diamandis and T. Christopoulus, Academic Press, Inc., San Diego, C A, 1996; and “Practice and Theory of Enzyme Immunoassays”, P. Tijssen, Elsevier Science Publishers, Amsterdam, 1985. Furthermore, in vivo techniques for detection of an analyte protein include introducing into a subject a labeled anti-analyte protein antibody. For example, the antibody can be labeled with a radioactive marker whose presence and location in a subject can be detected by standard imaging techniques.

Chimeric Antigen Receptor (CAR) T-Cell Therapies

Cellular therapies, such as chimeric antigen receptor (CAR) T-cell therapies, are also provided herein. CAR T-cell therapies redirect a patient's T-cells to kill tumor cells by the exogenous expression of a CAR on a T-cell, for example. A CAR can be a membrane spanning fusion protein that links the antigen recognition domain of an antibody to the intracellular signaling domains of the T-cell receptor and co-receptor. A suitable cell can be used, for example, that can secrete an anti-SARS-CoV2 antibody of the present invention (or alternatively engineered to express an anti-SARS-CoV2 antibody as described herein to be secreted). The anti-SARS-CoV2 “payloads” to be secreted, can be, for example, minibodies, scFvs, IgG molecules, bispecific fusion molecules, and other antibody fragments as described herein. Upon contact or engineering, the cell described herein can then be introduced to a patient in need of a treatment by infusion therapies known to one of skill in the art. The patient may have a SARS-CoV2 disease, such as COVID-19. The cell (e.g., a T cell) can be, for instance, T lymphocyte, a CD4+ T cell, a CD8+ T cell, or the combination thereof, without limitation. Exemplary CARs and CAR factories useful in aspects of the invention include those disclosed in, for example, PCT/US2015/067225 and PCT/US2019/022272, each of which are hereby incorporated by reference in their entireties. In one embodiment, the SARS-CoV2 antibodies discussed herein can be used in the construction of the payload for a CAR-T cell. For example, in one embodiment, the anti-SARS-CoV2 antibodies discussed herein can be used for the targeting of the CARS (i.e., as the targeting moiety). In another embodiment, the anti-SARS-CoV2 antibodies discussed herein can be used as the targeting moiety, and a different SARS-CoV2 antibody that targets a different epitope can be used as the payload. In another embodiment, the payload can be an immunomodulatory antibody payload.

Pharmaceutical Compositions

The antibodies or agents of the invention (also referred to herein as “active compounds”), and derivatives, fragments, analogs and homologs thereof, can be incorporated into pharmaceutical compositions suitable for administration. Such compositions typically comprise the antibody or agent and a pharmaceutically acceptable carrier. As used herein, the term “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Non-limiting examples of such carriers or diluents include, but are not limited to, water, saline, ringer's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be useful to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Dispersions can be prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

In embodiments, compositions, such as oral or parenteral compositions, can be formulated in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the therapeutic effect to be achieved, and the limitations inherent in the art of compounding such an active compound for the treatment of individuals.

The pharmaceutical compositions can be included in a container, pack, or dispenser together with instructions for administration.

Screening Methods

The invention provides methods (also referred to herein as “screening assays”) for identifying modulators, i.e., candidate or test compounds or agents (e.g., peptides, peptidomimetics, small molecules or other drugs) that modulate or otherwise interfere with the fusion of a SARS-CoV2 to the cell membrane. Also provided are methods of identifying compounds useful to treat SARS-CoV2 infection. The invention also encompasses compounds identified using the screening assays described herein.

For example, the invention provides assays for screening candidate or test compounds which modulate the interaction between the SARS-CoV2 and the cell membrane. The test compounds of the invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds. (See, e.g., Lam, 1997. Anticancer Drug Design 12: 145).

A “small molecule” as used herein, is meant to refer to a composition that has a molecular weight of less than about 5 kD, for example less than about 4 kD. Small molecules can be, e.g., nucleic acids, peptides, polypeptides, peptidomimetics, carbohydrates, lipids or other organic or inorganic molecules. Libraries of chemical and/or biological mixtures, such as fungal, bacterial, or algal extracts, are known in the art and can be screened with any of the assays of the invention.

Examples of methods for the synthesis of molecular libraries can be found in the art, for example in: DeWitt, et al., 1993. Proc. Natl. Acad. Sci. U.S.A. 90: 6909; Erb, et al., 1994. Proc. Natl. Acad. Sci. U.S.A. 91: 11422; Zuckermann, et al., 1994. J. Med. Chem. 37: 2678; Cho, et al., 1993. Science 261: 1303; Carrell, et al., 1994. Angew. Chem. Int. Ed. Engl. 33: 2059; Carell, et al., 1994. Angew. Chem. Int. Ed. Engl. 33: 2061; and Gallop, et al., 1994. J. Med. Chem. 37: 1233.

Libraries of compounds can be presented in solution (see e.g., Houghten, 1992, Biotechniques 13: 412-421), or on beads (see Lam, 1991. Nature 354: 82-84), on chips (see Fodor, 1993. Nature 364: 555-556), bacteria (see U.S. Pat. No. 5,223,409), spores (see U.S. Pat. No. 5,233,409), plasmids (see Cull, et al., 1992, Proc. Natl. Acad. Sci. USA 89: 1865-1869) or on phage (see Scott and Smith, 1990, Science 249: 386-390; Devlin, 1990, Science 249: 404-406; Cwirla, et al., 1990, Proc. Natl. Acad. Sci. U.S.A. 87: 6378-6382; Felici, 1991, J. Mol. Biol. 222: 301-310; and U.S. Pat. No. 5,233,409.).

In one embodiment, a candidate compound is introduced to an antibody-antigen complex and determining whether the candidate compound disrupts the antibody-antigen complex, wherein a disruption of this complex indicates that the candidate compound modulates the interaction between a SARS-CoV2 and the cell membrane.

In another embodiment, at least one SARS-CoV2 protein is provided, which is exposed to at least one neutralizing monoclonal antibody. Formation of an antibody-antigen complex is detected, and one or more candidate compounds are introduced to the complex. If the antibody-antigen complex is disrupted following introduction of the one or more candidate compounds, the candidate compounds is useful to treat a SARS-CoV2-related disease or disorder. For example, the at least one SARS-CoV2 protein can be provided as a SARS-CoV2 molecule.

Determining the ability of the test compound to interfere with or disrupt the antibody-antigen complex can be accomplished, for example, by coupling the test compound with a radioisotope or enzymatic label such that binding of the test compound to the antigen or biologically-active portion thereof can be determined by detecting the labeled compound in a complex. For example, test compounds can be labeled with ¹²¹I, ³⁵S, ¹⁴C, or ³H, either directly or indirectly, and the radioisotope detected by direct counting of radioemission or by scintillation counting. Alternatively, test compounds can be enzymatically-labeled with, for example, horseradish peroxidase, alkaline phosphatase, or luciferase, and the enzymatic label detected by determination of conversion of an appropriate substrate to product.

In one embodiment, the assay comprises contacting an antibody-antigen complex with a test compound, and determining the ability of the test compound to interact with the antigen or otherwise disrupt the existing antibody-antigen complex. In this embodiment, determining the ability of the test compound to interact with the antigen and/or disrupt the antibody-antigen complex comprises determining the ability of the test compound to bind to the antigen or a biologically-active portion thereof, as compared to the antibody.

In another embodiment, the assay comprises contacting an antibody-antigen complex with a test compound and determining the ability of the test compound to modulate the antibody-antigen complex. Determining the ability of the test compound to modulate the antibody-antigen complex can be accomplished, for example, by determining the ability of the antigen to bind to or interact with the antibody, in the presence of the test compound.

Those skilled in the art will recognize that, in any of the screening methods disclosed herein, the antibody can be a SARS-CoV2 neutralizing antibody or any variant thereof wherein the Fc region is modified such that it has reduced binding or does not bind to the Fc-gamma receptor. Additionally, the antigen can be a SARS-CoV2 protein, or a portion thereof.

The screening methods disclosed herein can be performed as a cell-based assay or as a cell-free assay. The cell-free assays of the invention are amenable to use of both the soluble form or the membrane-bound form of the proteins and fragments thereof. In the case of cell-free assays comprising the membrane-bound forms of the proteins, it may be desirable to utilize a solubilizing agent such that the membrane-bound form of the proteins are maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, N-dodecyl-N,N-dimethyl-3-ammonio-1-propane sulfonate, 3-(3-cholamidopropyl) dimethylamminiol-1-propane sulfonate (CHAPS), or 3-(3-cholamidopropyl)dimethylamminiol-2-hydroxy-1-propane sulfonate (CHAPSO).

In more than one embodiment, it can be desirable to immobilize either the antibody or the antigen to facilitate separation of complexed from uncomplexed forms of one or both following introduction of the candidate compound, as well as to accommodate automation of the assay. Observation of the antibody-antigen complex in the presence and absence of a candidate compound can be accomplished in any vessel suitable for containing the reactants. Examples of such vessels include microtiter plates, test tubes, and micro-centrifuge tubes. In one embodiment, a fusion protein can be provided that adds a domain that allows one or both of the proteins to be bound to a matrix. For example, GST-antibody fusion proteins or GST-antigen fusion proteins can be adsorbed onto glutathione Sepharose beads (Sigma Chemical, St. Louis, MO) or glutathione derivatized microtiter plates, that are then combined with the test compound, and the mixture is incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly. Alternatively, the complexes can be dissociated from the matrix, and the level of antibody-antigen complex formation can be determined using standard techniques.

Other techniques for immobilizing proteins on matrices can also be used in the screening assays of the invention. For example, either the antibody or the antigen (e.g. the can be immobilized utilizing conjugation of biotin and streptavidin. Biotinylated antibody or antigen molecules can be prepared from biotin-NHS (N-hydroxy-succinimide) using techniques well-known within the art (e.g., biotinylation kit, Pierce Chemicals, Rockford, Ill.), and immobilized in the wells of streptavidin-coated 96 well plates (Pierce Chemical). Alternatively, other antibodies reactive with the antibody or antigen of interest, but which do not interfere with the formation of the antibody-antigen complex of interest, can be derivatized to the wells of the plate, and unbound antibody or antigen trapped in the wells by antibody conjugation. Methods for detecting such complexes, in addition to those described above for the GST-immobilized complexes, include immunodetection of complexes using such other antibodies reactive with the antibody or antigen.

The invention further pertains to new agents identified by any of the aforementioned screening assays and uses thereof for treatments as described herein.

Diagnostic Assays

Antibodies of the present invention can be detected by or used for detection purposes by appropriate assays, e.g., conventional types of immunoassays such as sandwich ELISAs. For example, an assay can be performed in which a SARS-CoV2 or fragment thereof is affixed to a solid phase. Incubation is maintained for a sufficient period of time to allow the antibody in the sample to bind to the immobilized polypeptide on the solid phase. After this first incubation, the solid phase is separated from the sample. The solid phase is washed to remove unbound materials and interfering substances such as non-specific proteins which may also be present in the sample. The solid phase containing the antibody of interest bound to the immobilized polypeptide is subsequently incubated with a second, labeled antibody or antibody bound to a coupling agent such as biotin or avidin. This second antibody can be another anti-SARS-CoV2 antibody or another antibody. Labels for antibodies are well-known in the art and include radionuclides, enzymes (e.g. maleate dehydrogenase, horseradish peroxidase, glucose oxidase, catalase), fluors (fluorescein isothiocyanate, rhodamine, phycocyanin, fluorescarmine), biotin, and the like. The labeled antibodies are incubated with the solid and the label bound to the solid phase is measured. These and other immunoassays can be easily performed by those of ordinary skill in the art.

An exemplary method for detecting the presence or absence of a SARS-CoV2 in a biological sample involves obtaining a biological sample from a test subject and contacting the biological sample with a labeled monoclonal antibody according to the invention such that the presence of the SARS-CoV2 is detected in the biological sample.

As used herein, the term “labeled”, with regard to the probe or antibody, can refer to direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” can refer to tissues, cells and biological fluids isolated from a subject, as well as tissues, cells and fluids present within a subject. That is, the detection method of the invention can be used to detect a SARS-CoV2 in a biological sample in vitro as well as in vivo. For example, in vitro techniques for detection of a SARS-CoV2 include enzyme linked immunosorbent assays (ELISAs), Western blots, immunoprecipitations, and immunofluorescence. Furthermore, in vivo techniques for detection of a SARS-CoV2 include introducing into a subject a labeled anti-SARS-CoV2 antibody. For example, the antibody can be labeled with a radioactive marker whose presence and location in a subject can be detected by standard imaging techniques.

In one embodiment, the biological sample contains protein molecules from the test subject. For example, one biological sample is a peripheral blood leukocyte sample isolated by conventional means from a subject.

The invention also encompasses kits for detecting the presence of a SARS-CoV2 in a biological sample. For example, the kit can comprise: a labeled compound or agent capable of detecting a SARS-CoV2 (e.g., an anti-SARS-CoV2 monoclonal antibody) in a biological sample; means for determining the amount of a SARS-CoV2 in the sample; and means for comparing the amount of a SARS-CoV2 in the sample with a standard. The compound or agent can be packaged in a suitable container. The kit can further comprise instructions for using the kit to detect a SARS-CoV2 in a sample.

Passive Immunization

Passive immunization has proven to be an effective and safe strategy for the prevention and treatment of viral diseases. (See Keller et al., Clin. Microbiol. Rev. 13:602-14 (2000); Casadevall, Nat. Biotechnol. 20:114 (2002); Shibata et al., Nat. Med. 5:204-10 (1999); and Igarashi et al., Nat. Med. 5:211-16 (1999), each of which are incorporated herein by reference)). Passive immunization using neutralizing human monoclonal antibodies could provide an immediate treatment strategy for emergency prophylaxis and treatment of SARS-CoV2 infection and related diseases and disorders while the alternative and more time-consuming development of vaccines and new drugs in underway.

Subunit vaccines potentially offer significant advantages over conventional immunogens. They avoid the safety hazards inherent in production, distribution, and delivery of conventional killed or attenuated whole-pathogen vaccines. Furthermore, they can be rationally designed to include only confirmed protective epitopes, thereby avoiding suppressive T epitopes (see Steward et al., J. Virol. 69:7668 (1995)) or immunodominant B epitopes that subvert the immune system by inducing futile, non-protective responses (e.g. “decoy” epitopes). (See Garrity et al., J. Immunol. 159:279 (1997)).

Moreover, those skilled in the art will recognize that good correlation exists between the antibody neutralizing activity in vitro and the protection in vivo for many different viruses, challenge routes, and animal models. (See Burton, Natl. Rev. Immunol. 2:706-13 (2002); Parren et al., Adv. Immunol. 77:195-262 (2001)).

Antigen-Ig Chimeras in Vaccination

It has been over a decade since the first antibodies were used as scaffolds for the efficient presentation of antigenic determinants to the immune systems. (See Zanetti, Nature 355:476-77 (1992); Zaghouani et al., Proc. Natl. Acad. Sci. USA 92:631-35 (1995)). When a peptide is included as an integral part of an IgG molecule (e.g., the 11A or 256 IgG1 monoclonal antibody described herein), the antigenicity and immunogenicity of the peptide epitopes are greatly enhanced as compared to the free peptide. Such enhancement is possibly due to the antigen-IgG chimeras longer half-life, better presentation and constrained conformation, which mimic their native structures.

Moreover, an added advantage of using an antigen-Ig chimera is that either the variable or the Fc region of the antigen-Ig chimera can be used for targeting professional antigen-presenting cells (APCs). To date, recombinant Igs have been generated in which the complementarity-determining regions (CDRs) of the heavy chain variable gene (V_{H}) are replaced with various antigenic peptides recognized by B or T cells. Such antigen-Ig chimeras have been used to induce both humoral and cellular immune responses. (See Bona et al., Immunol. Today 19:126-33 (1998)).

Chimeras with specific epitopes engrafted into the CDR3 loop have been used to induce humoral responses to either HIV-1 gp120 V3-loop or the first extracellular domain (D1) of human CD4 receptor. (See Lanza et al., Proc. Natl. Acad. Sci. USA 90:11683-87 (1993); Zaghouani et al., Proc. Natl. Acad. Sci. USA 92:631-35 (1995)). The immune sera were able to prevent infection of CD4 SupTI cells by HIV-1MN (anti-gp120 V3C) or inhibit syncytia formation (anti-CD4-D1). The CDR2 and CDR3 can be replaced with peptide epitopes simultaneously, and the length of peptide inserted can be up to 19 amino acids long.

Alternatively, one group has developed a “troybody” strategy in which peptide antigens are presented in the loops of the Ig constant (C) region and the variable region of the chimera can be used to target IgD on the surface of B-cells or MHC class II molecules on professional APCs including B-cells, dendritic cells (DC) and macrophages. (See Lunde et al., Biochem. Soc. Trans. 30:500-6 (2002)).

An antigen-Ig chimera can also be made by directly fusing the antigen with the Fc portion of an IgG molecule. You et al., Cancer Res. 61:3704-11 (2001) were able to obtain all arms of specific immune response, including very high levels of antibodies to hepatitis B virus core antigen using this method.

DNA Vaccination

DNA vaccines are stable, can provide the antigen an opportunity to be naturally processed, and can induce a longer-lasting response. Although a very attractive immunization strategy, DNA vaccines often have very limited potency to induce immune responses. Poor uptake of injected DNA by professional APCs, such as dendritic cells (DCs), may be the main cause of such limitation. Combined with the antigen-Ig chimera vaccines, a promising new DNA vaccine strategy based on the enhancement of APC antigen presentation has been reported (see Casares, et al., Viral Immunol. 10:129-36 (1997); Gerloni et al., Nat. Biotech. 15:876-81 (1997); Gerloni et al., DNA Cell Biol. 16:611-25 (1997); You et al., Cancer Res. 61:3704-11 (2001)), which takes advantage of the presence of Fc receptors (FcγRs) on the surface of DCs.

An embodiment comprises a DNA vaccine encoding an antigen (Ag)-Ig chimera. Upon immunization, Ag-Ig fusion proteins will be expressed and secreted by the cells taking up the DNA molecules. The secreted Ag-Ig fusion proteins, while inducing B-cell responses, can be captured and internalized by interaction of the Fc fragment with FcγRs on DC surface, which will promote efficient antigen presentation and greatly enhance antigen-specific immune responses. Applying the same principle, DNA encoding antigen-Ig chimeras carrying a functional anti-MHC II specific scFv region gene can also target the immunogens to all three types of APCs. The immune responses could be further boosted with use of the same protein antigens generated in vitro (i.e., “prime and boost”), if necessary. Using this strategy, specific cellular and humoral immune responses against infection of SARS-CoV2 were accomplished through intramuscular (i.m.) injection of a DNA vaccine. (See Casares et al., Viral. Immunol. 10:129-36 (1997)).

Vaccine Compositions

Therapeutic or prophylactic compositions are provided herein, which generally comprise mixtures of one or more monoclonal antibodies or ScFvs and combinations thereof. The prophylactic vaccines can be used to prevent a SARS-CoV2 infection and the therapeutic vaccines can be used to treat individuals following a SARS-CoV2 infection. Prophylactic uses include the provision of increased antibody titer to a SARS-CoV2 in a vaccination subject. In this manner, subjects at high risk of contracting SARS-CoV2 can be provided with passive immunity to a SARS-CoV2.

These vaccine compositions can be administered in conjunction with ancillary immunoregulatory agents. For example, cytokines, lymphokines, and chemokines, including, but not limited to, IL-2, modified IL-2 (Cys125→Ser125), GM-CSF, IL-12, γ-interferon, IP-10, MIP1β, and RANTES.

Methods of Immunization

The vaccines of the present invention have superior immunoprotective and immunotherapeutic properties over other anti-viral vaccines.

The invention provides a method of immunization, e.g., inducing an immune response, of a subject. A subject is immunized by administration to the subject a composition containing a membrane fusion protein of a pathogenic spike protein. The fusion protein is coated or embedded in a biologically compatible matrix.

The fusion protein is glycosylated, e.g. contains a carbohydrate moiety. The carbohydrate moiety may be in the form of a monosaccharide, disaccharide(s). oligosaccharide(s), polysaccharide(s), or their derivatives (e.g. sulfo- or phospho-substituted). The carbohydrate is linear or branched. The carbohydrate moiety is N-linked or O-linked to a polypeptide. N-linked glycosylation is to the amide nitrogen of asparagine side chains and O-linked glycosylation is to the hydroxy oxygen of serine and threonine side chains.

The carbohydrate moiety is endogenous to the subject being vaccinated. Alternatively, the carbohydrate moiety is exogenous to the subject being vaccinated. The carbohydrate moiety is a carbohydrate moieties that are not typically expressed on polypeptides of the subject being vaccinated. For example, the carbohydrate moieties are plant-specific carbohydrates. Plant specific carbohydrate moieties include for example N-linked glycan having a core bound α1,3 fucose or a core bound β1,2 xylose. Alternatively, the carbohydrate moiety are carbohydrate moieties that are expressed on polypeptides or lipids of the subject being vaccinate. For example, many host cells have been genetically engineered to produce human proteins with human-like sugar attachments.

The subject is at risk of developing or suffering from a viral infection. For example, the subject has traveled to regions or countries in which other SARS-CoV2 infections have been reported.

The methods described herein lead to a reduction in the severity or the alleviation of one or more symptoms of a viral infection. Infections are diagnosed and or monitored, typically by a physician using standard methodologies. A subject requiring immunization is identified by methods know in the art. For example, subjects are immunized as outlined in the CDC's General Recommendation on Immunization (51(RR02) pp 1-36).

The subject is e.g., any mammal, e.g., a human, a primate, mouse, rat, dog, cat, camel, cow, horse, pig, a fish or a bird.

The treatment is administered prior to diagnosis of the infection. Alternatively, treatment is administered after diagnosis. Efficaciousness of treatment is determined in association with any known method for diagnosing or treating the disorder or infection. Alleviation of one or more symptoms of the disorder indicates that the compound confers a clinical benefit.

Methods of Treatment

As used herein, the terms “treat” or “treatment” refer to both therapeutic treatment and prophylactic or preventative measures, wherein the object is to prevent or slow down (lessen) an undesired physiological change or disorder, such as the progression of COVID. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can refer to prolonging survival as compared to expected survival if not receiving treatment. Those in need of treatment include those already with the condition or disorder as well as those prone to have the condition or disorder or those in which the condition or disorder is to be prevented. The invention provides for both prophylactic and therapeutic methods of treating a subject at risk of (or susceptible to) a SARS-CoV2-related disease or disorder.

Prophylactic Methods

In one aspect, the invention provides methods for preventing a SARS-CoV2-related disease or disorder in a subject by administering to the subject a monoclonal antibody of the invention or an agent identified according to the methods of the invention. For example, monoclonal antibodies of the invention, and any variants thereof, can be administered in therapeutically effective amounts. Optionally, two or more anti-SARS-CoV2 antibodies are co-administered. For example, the invention provides for both prophylactic and therapeutic methods of treating a subject at risk of (or susceptible to) COVID.

Subjects at risk for a SARS-CoV2-related diseases or disorders include patients who have been exposed to the SARS-CoV2. For example, the subjects have traveled to regions or countries of the world in which other SARS-CoV2 infections have been reported and confirmed. Administration of a prophylactic agent can occur prior to the manifestation of symptoms characteristic of the SARS-CoV2-related disease or disorder, such that a disease or disorder is prevented or, alternatively, delayed in its progression.

The appropriate agent can be determined based on screening assays described herein. Alternatively, or in addition, the agent to be administered is a monoclonal antibody that neutralizes a SARS-CoV2 that has been identified according to the methods of the invention. In some embodiments, the antibody of the present invention can be administered with other antibodies or antibody fragments known to neutralize SARS-CoV2. Administration of said antibodies can be sequential, concurrent, or alternating.

Therapeutic Methods

Another aspect of the invention pertains to methods of treating a SARS-CoV2-related disease or disorder in a patient. In one embodiment, the method involves administering an agent (e.g., an agent identified by a screening assay described herein and/or monoclonal antibody identified according to the methods of the invention), or combination of agents that neutralize the SARS-CoV2 to a patient suffering from the disease or disorder.

Combinatory Methods

The invention provides treating a SARS-CoV2-related disease or disorder, in a patient by administering two or more antibodies wherein the Fc region of said variant does not bind or has reduced binding to the Fc gamma receptor, with other SARS-CoV2 neutralizing antibodies known in the art. In another embodiment, the invention provides methods for treating a SARS-CoV2-related disease or disorder in a patient by administering an antibody of the present invention with any anti-viral agent known in the art. Anti-viral agents can be peptides, nucleic acids, small molecules, inhibitors, or RNAi.

The invention will be further described in the following examples, which do not limit the scope of the invention described in the claims.

### EXAMPLES

### Example 1

Purified Phage Binding Curves (RBD-Fc)

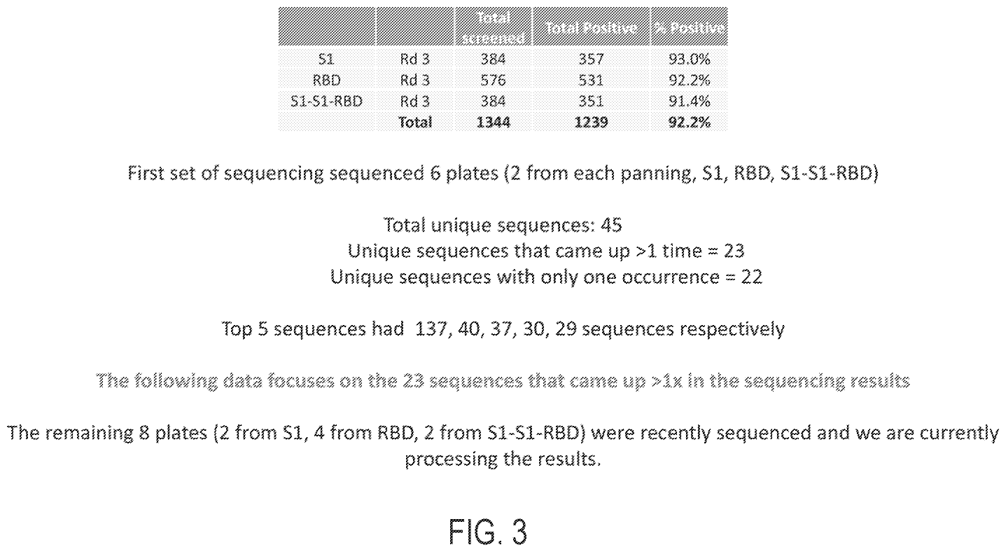

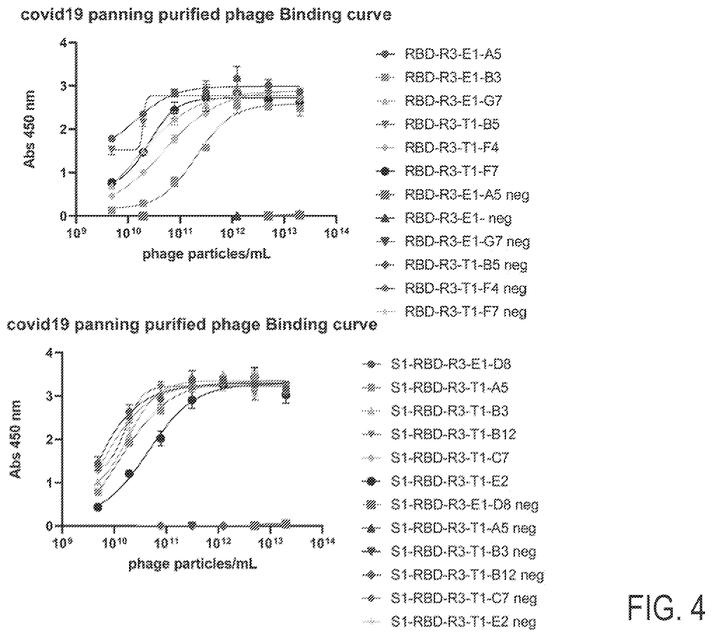

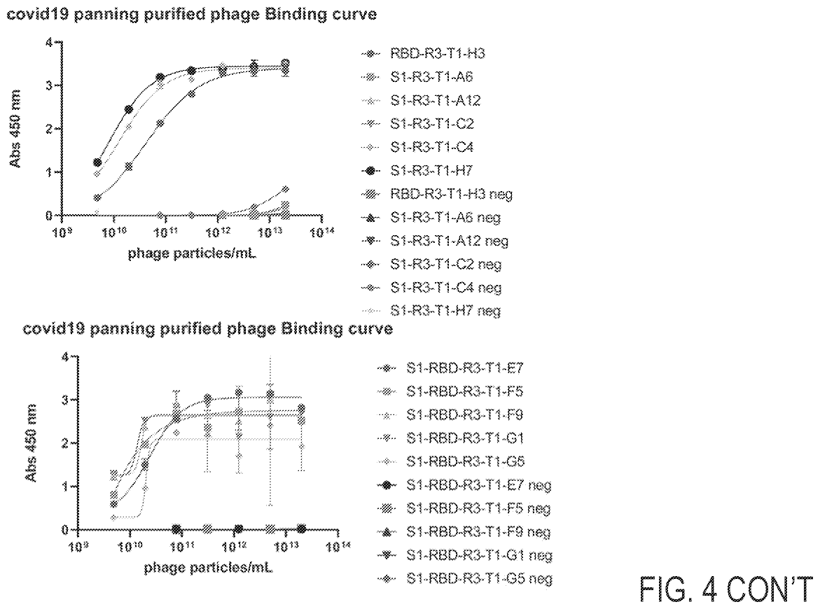

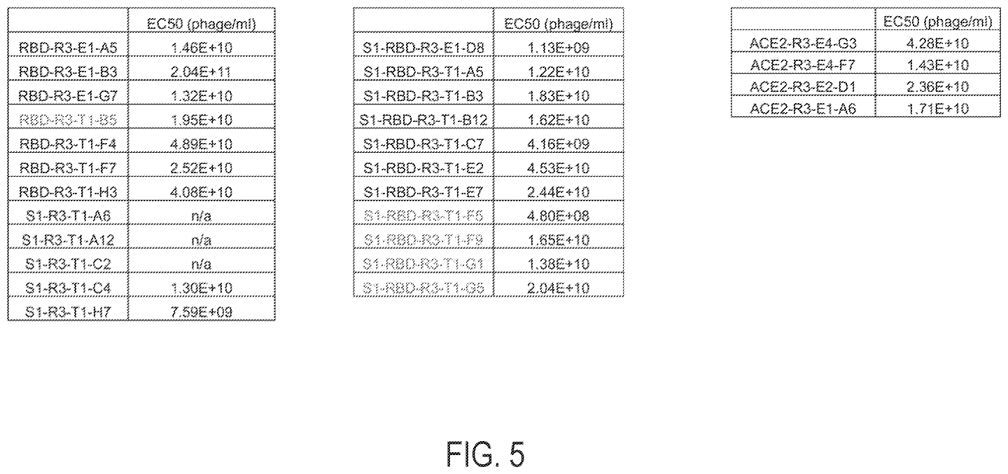

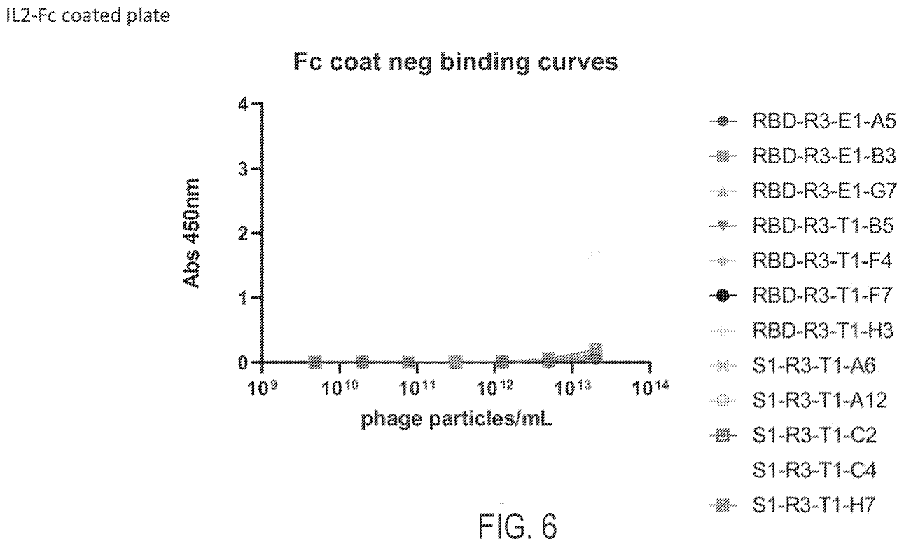

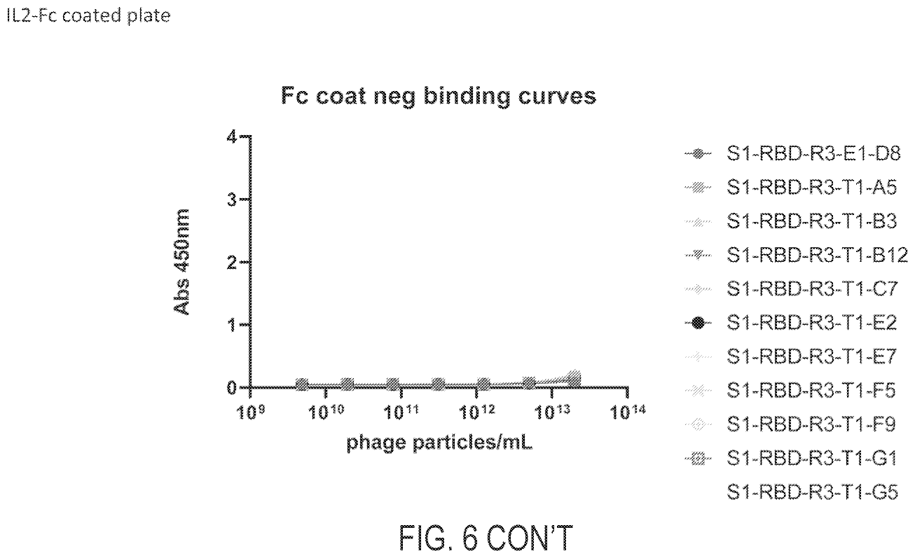

Based on the binding curves of FIG. 3-5, we have antibodies against the RBD with a variety of affinities. There are also 3 clones which do not bind to the RBD. Looking at the sequencing, there were multiple copies of each of these clones, but they only came from S1 panning plates. This indicates that they are S1 specific but not directed to the RBD So binding curves for those 3 were generated against S1 proteins.

### Example 2

Anti-RBD Competition with ACE2

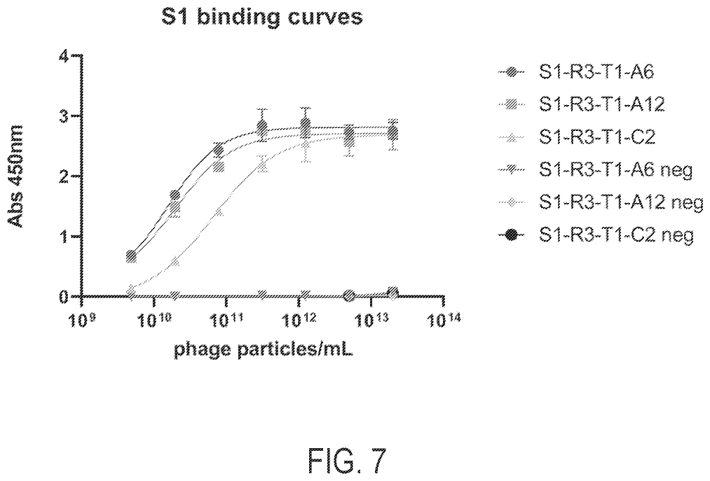

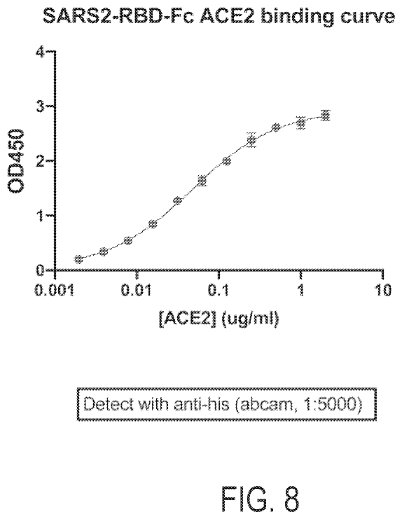

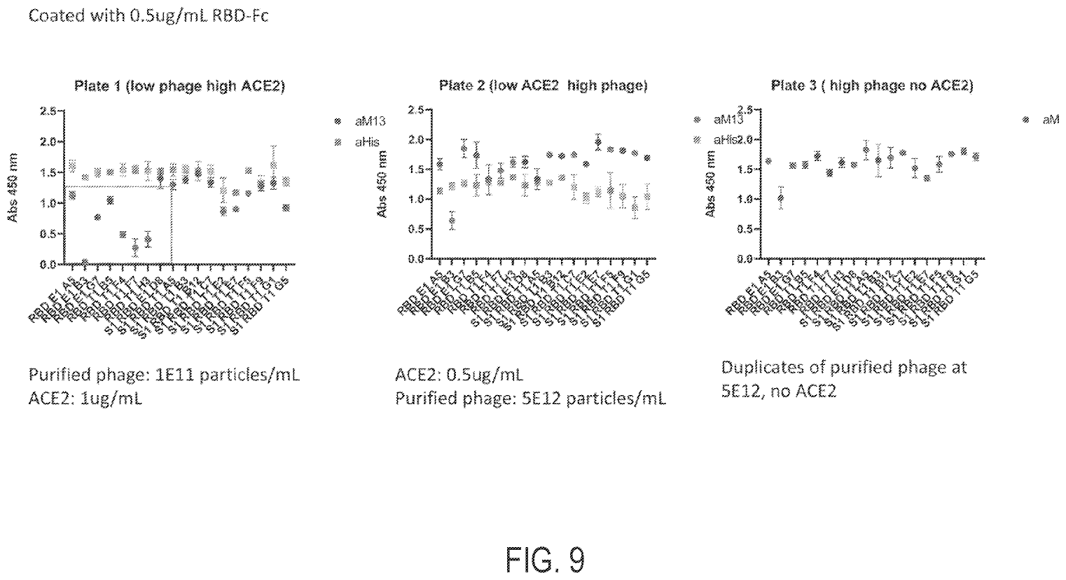

See, for example, FIG. 7-9.

Plates are coated with RBD-Fc at 0.5 ug/ml.

Plate 1: a low concentration of purified phage (on upper shoulder of binding curve) is first added to the plate, before a high concentration of ACE2 (1 μg/ml) is added

Plate 2: a low concentration of ACE2 (0.5 μg/ml) is first added to the plate, before a high concentration of purified phage is added

Samples were run in quadruplicate so that both phage binding (anti-M13) and ACE2 binding (anti-his) could be detected in duplicate

Based on the data shown in FIG. 7-9, for example, three clones were chosen for purified phage competition curves with ACE2.

Plates were coated with 0.5 μg/ml RBD-Fc. A constant amount of phage was added to each well (top shoulder of binding curve) followed by serial dilutions of ACE2. The remaining phage were then detected by anti-M13.

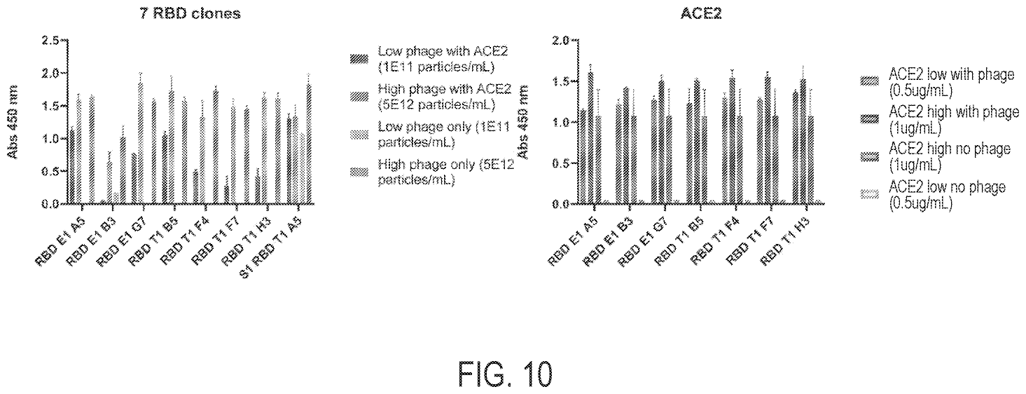

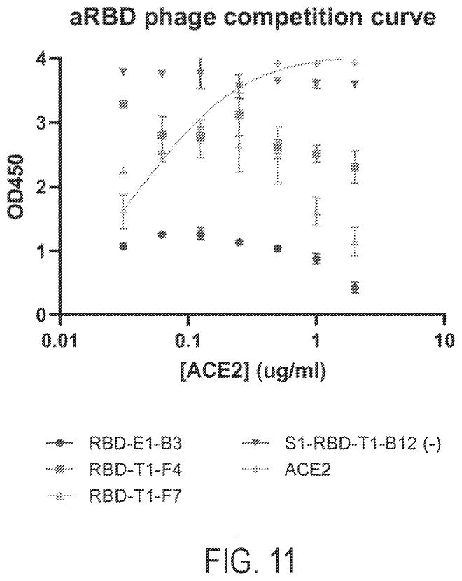

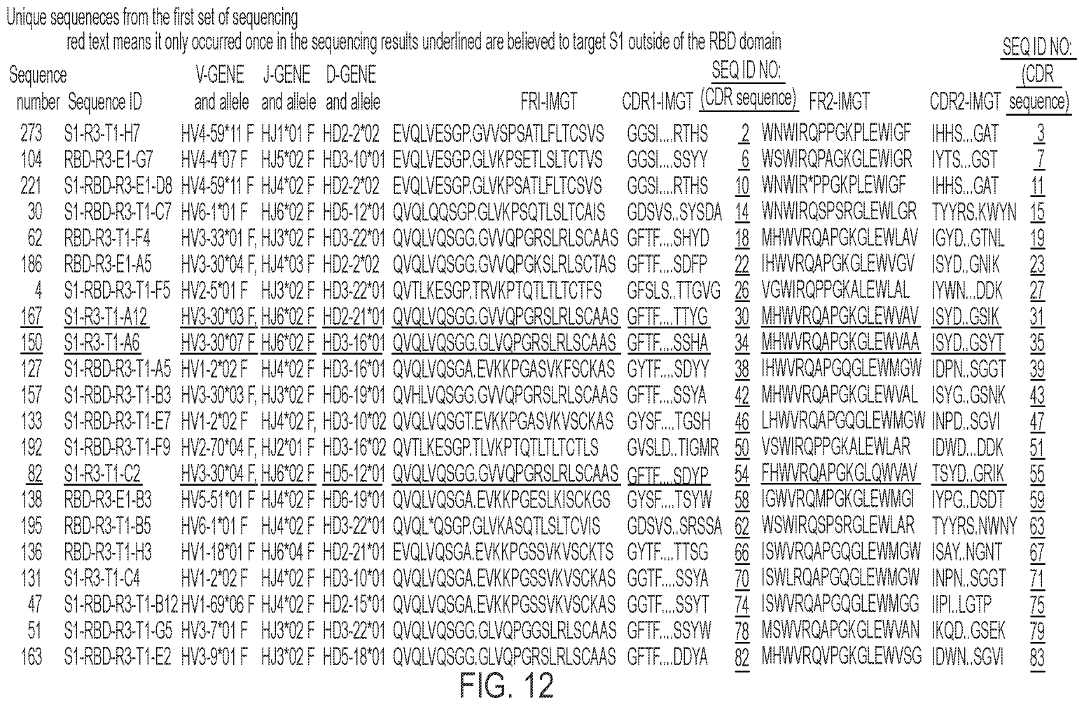

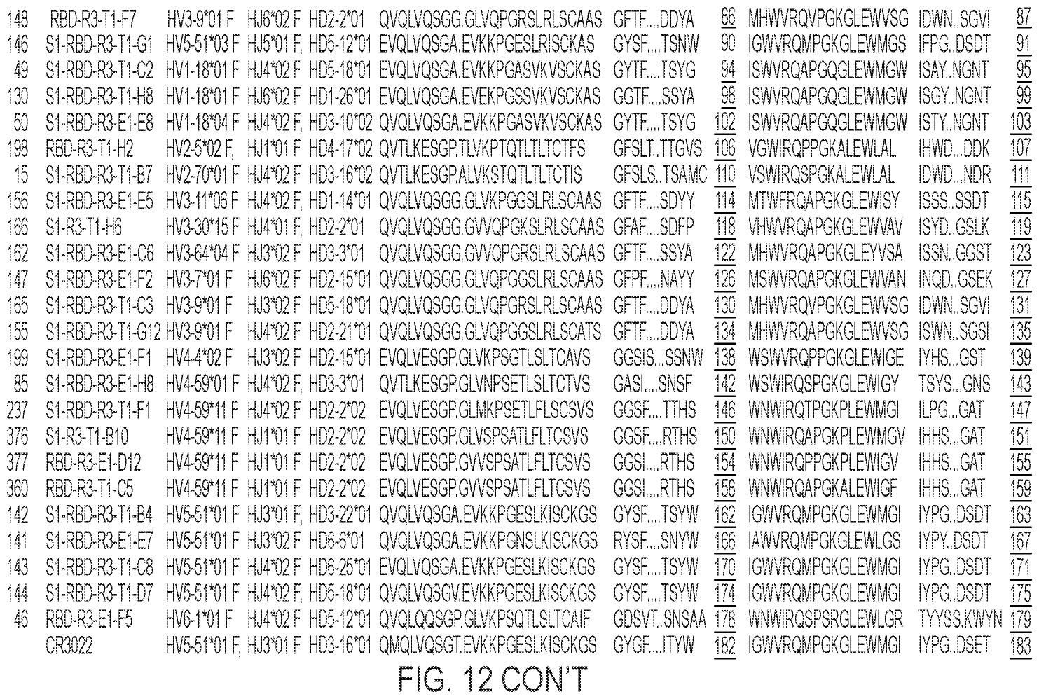

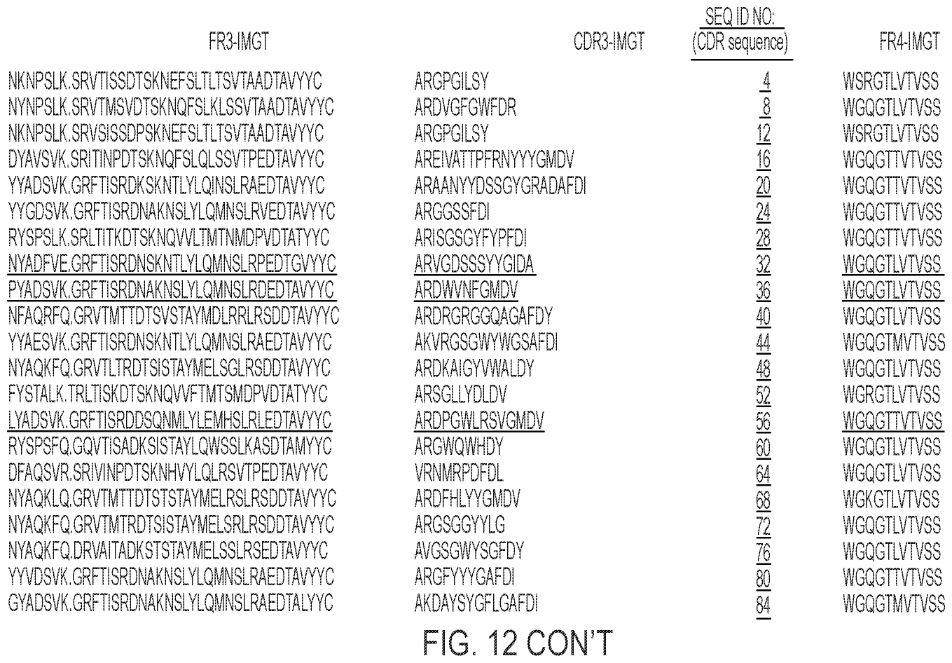

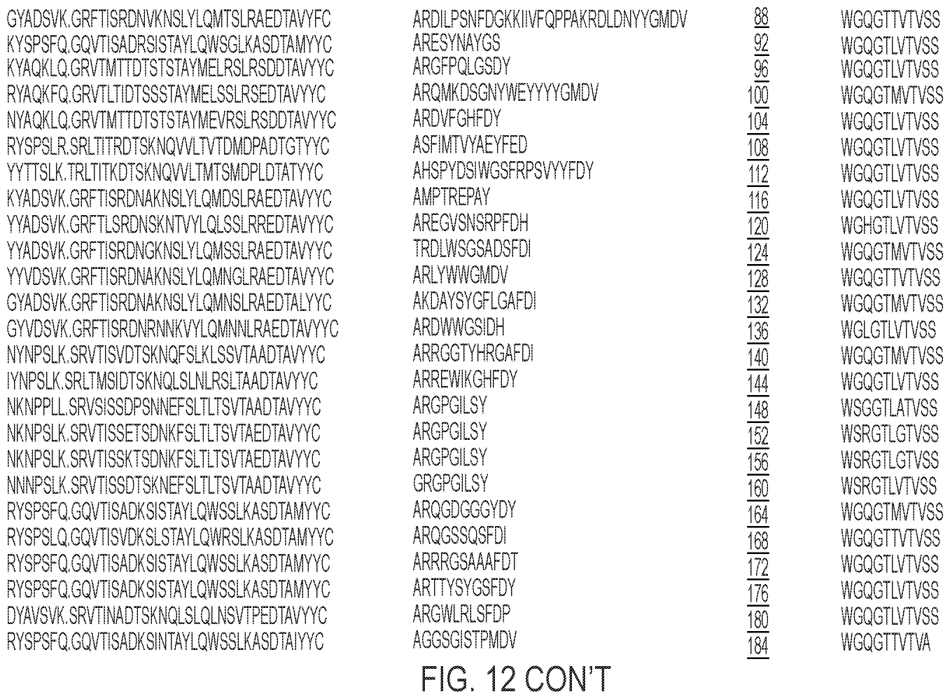

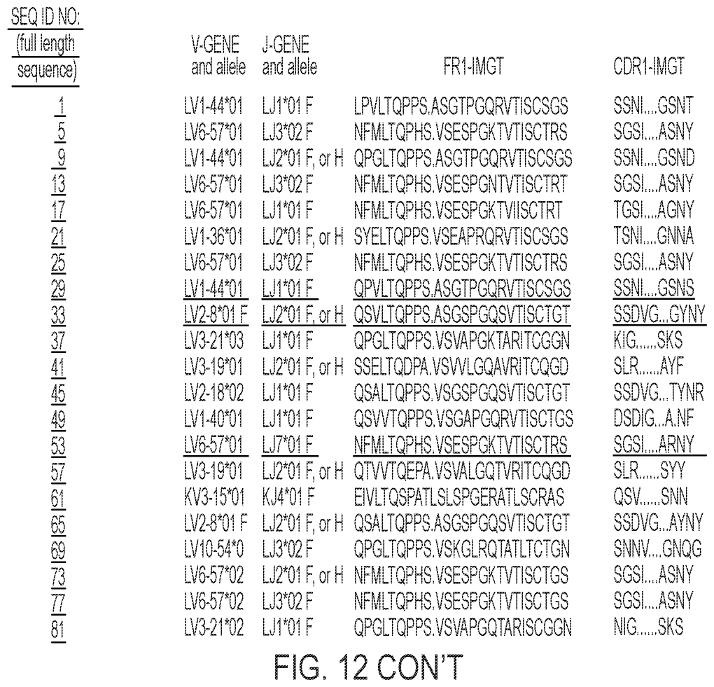

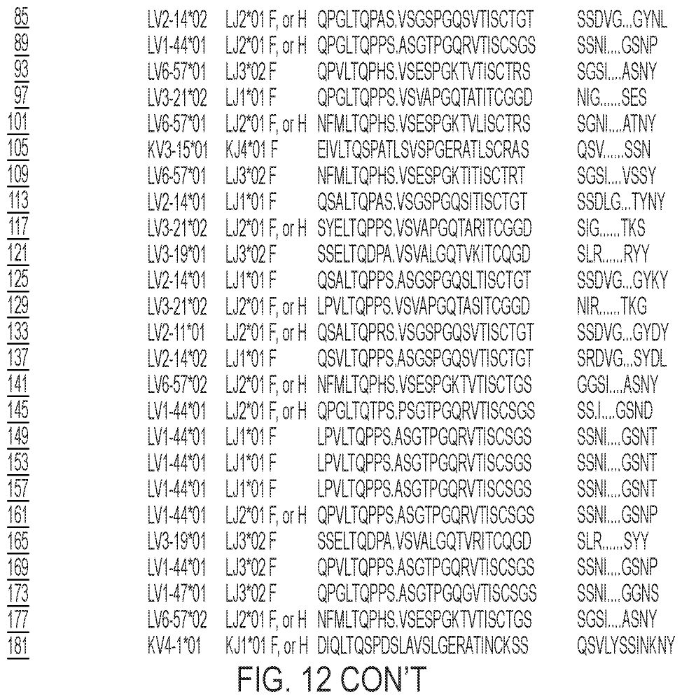

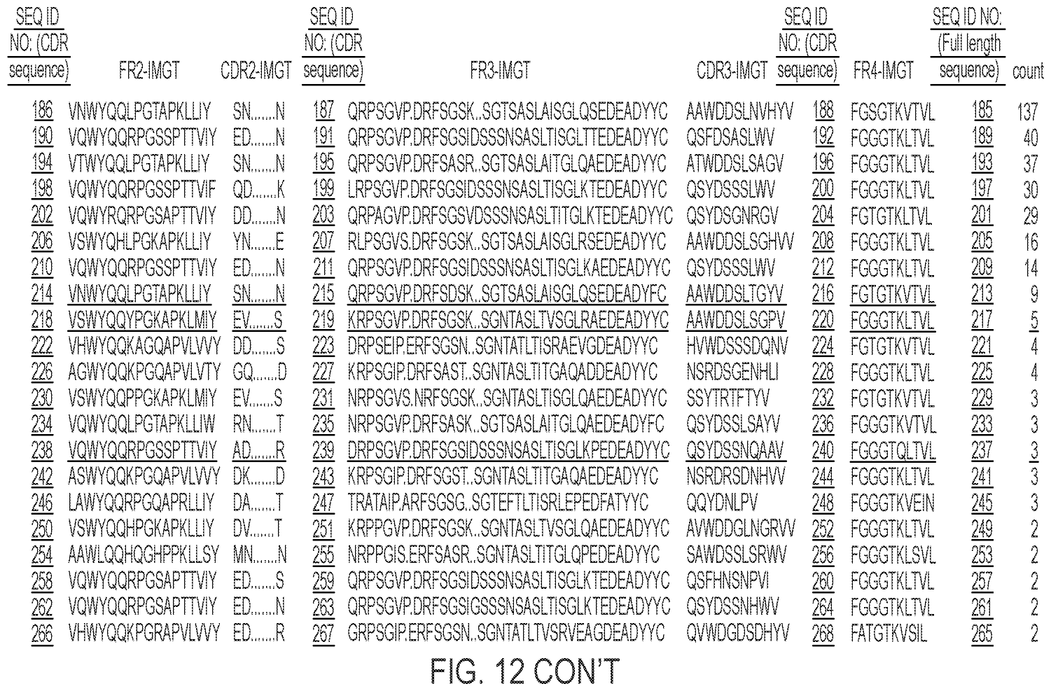

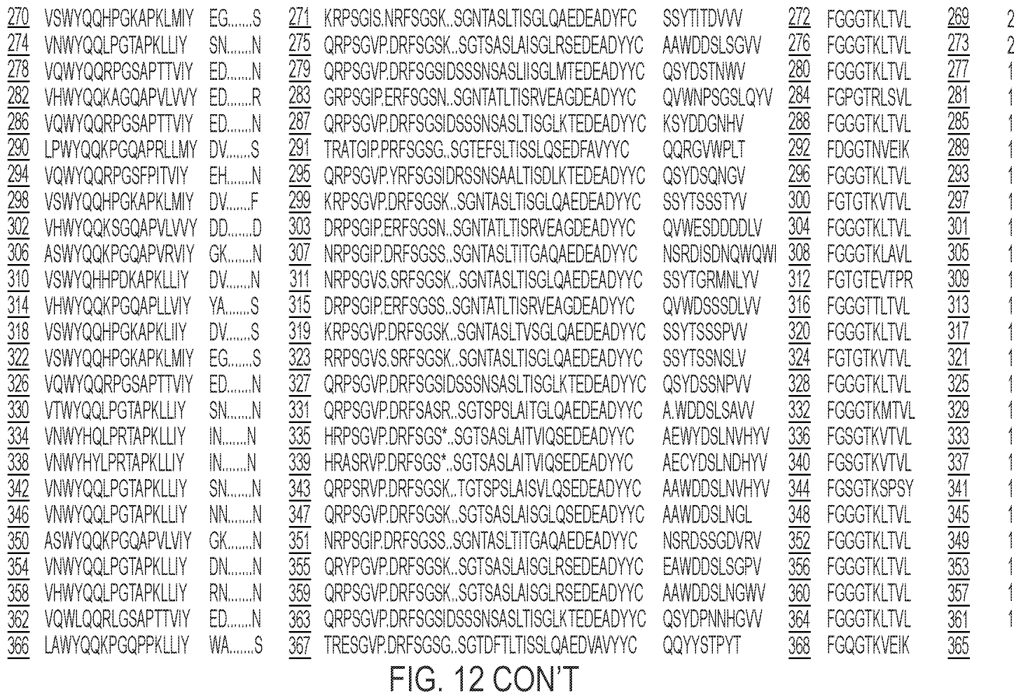

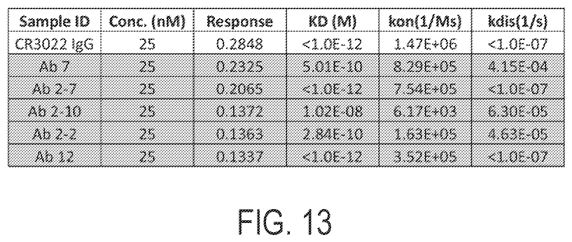

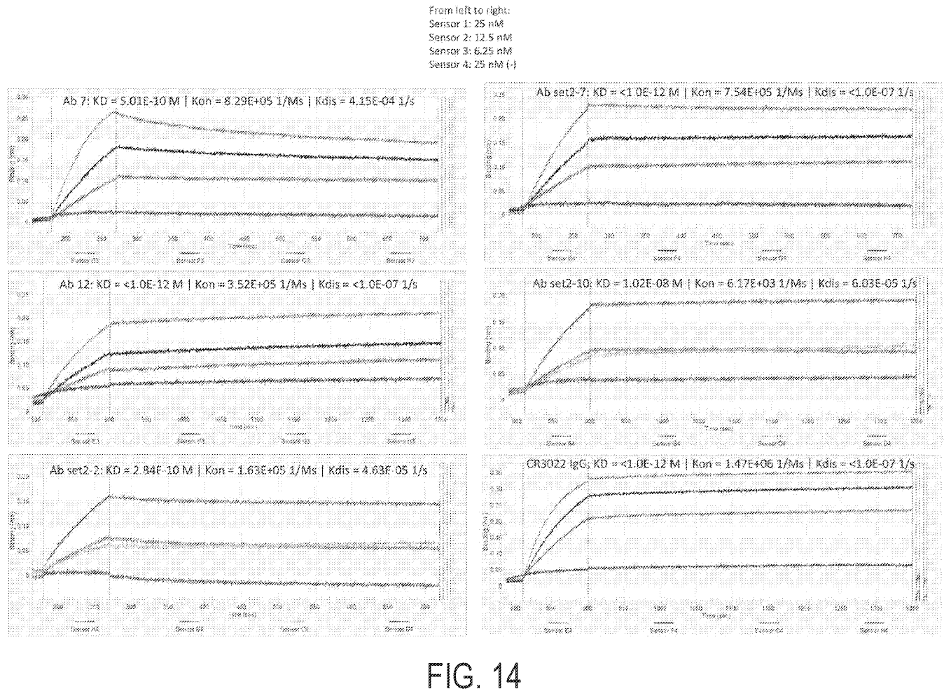

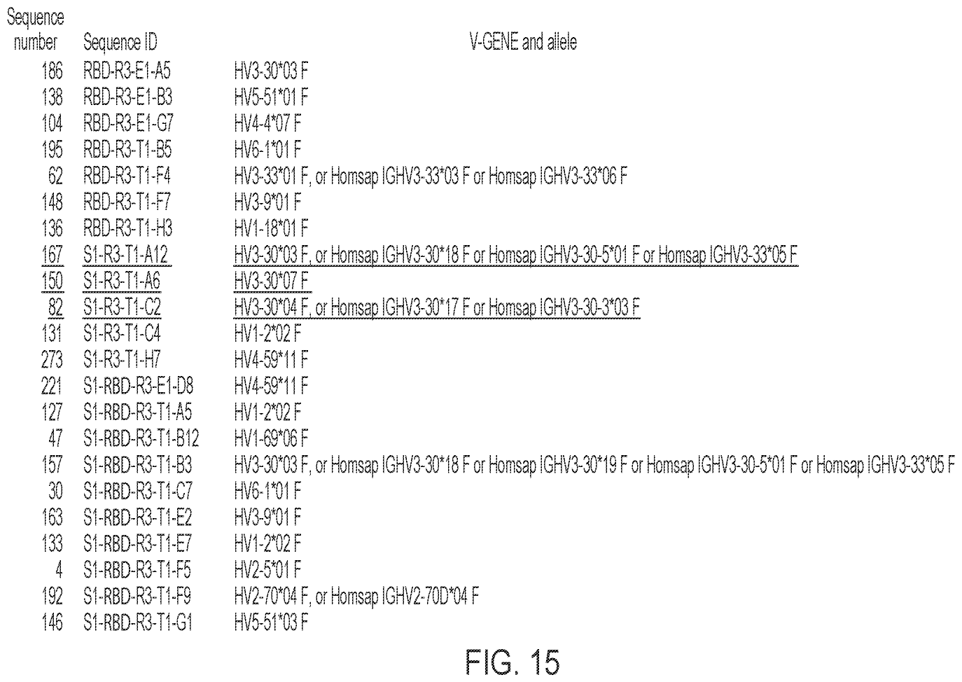

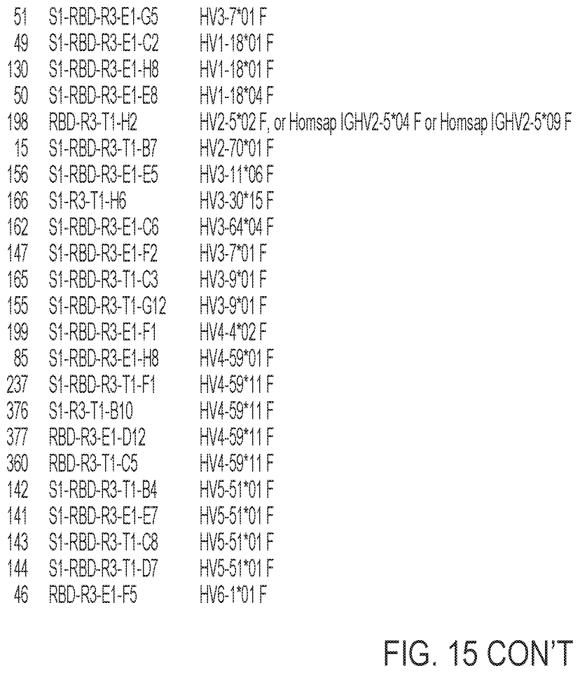

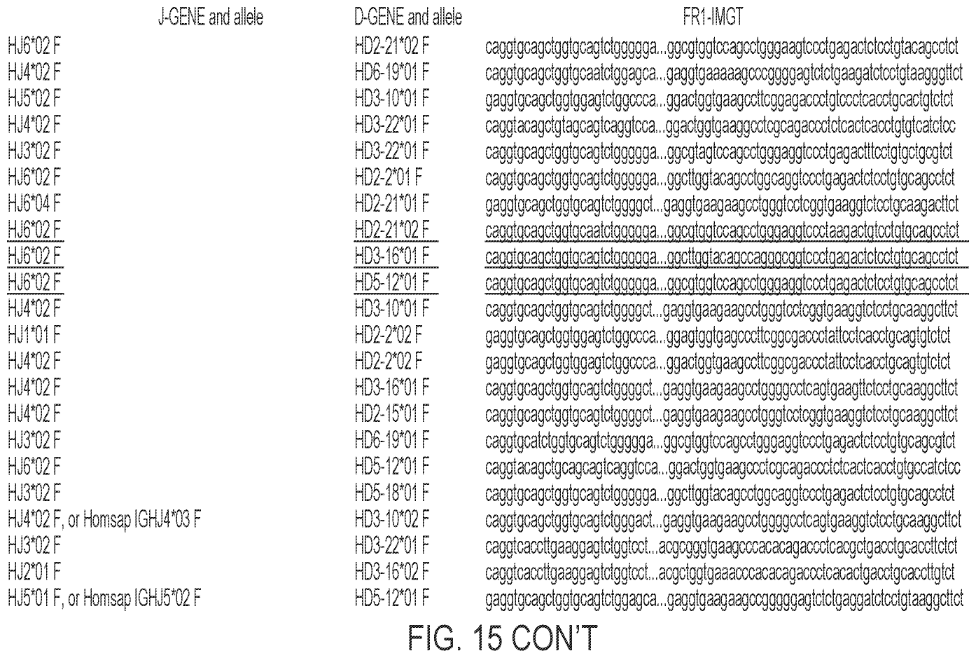

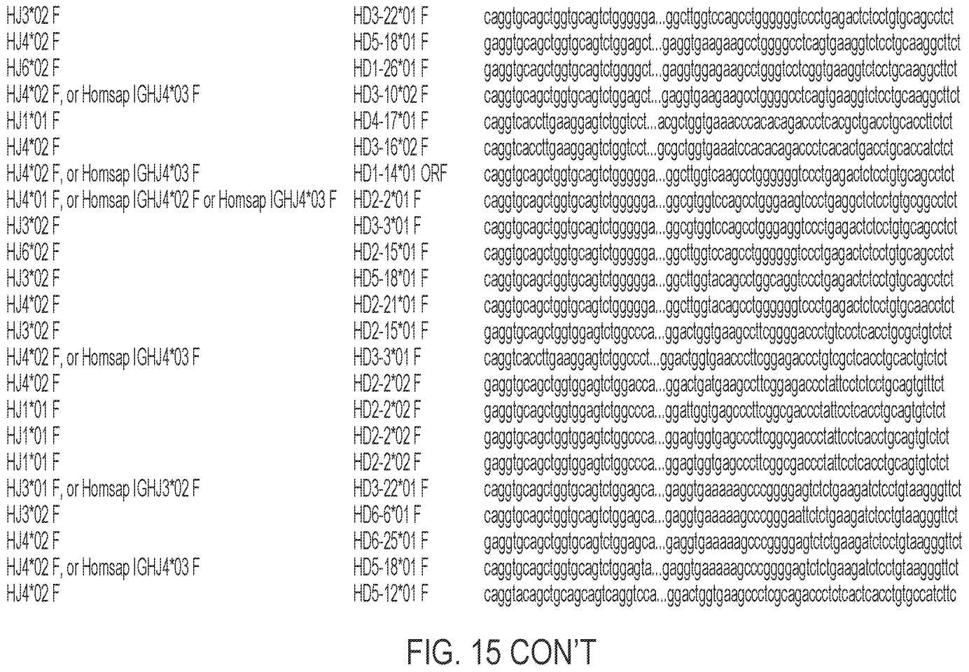

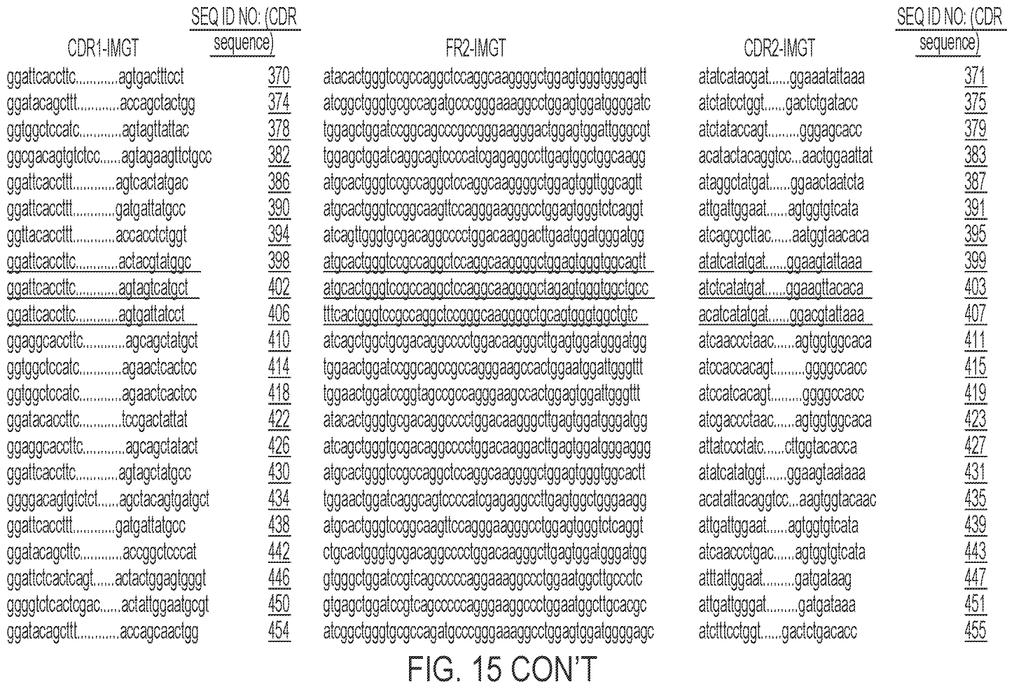

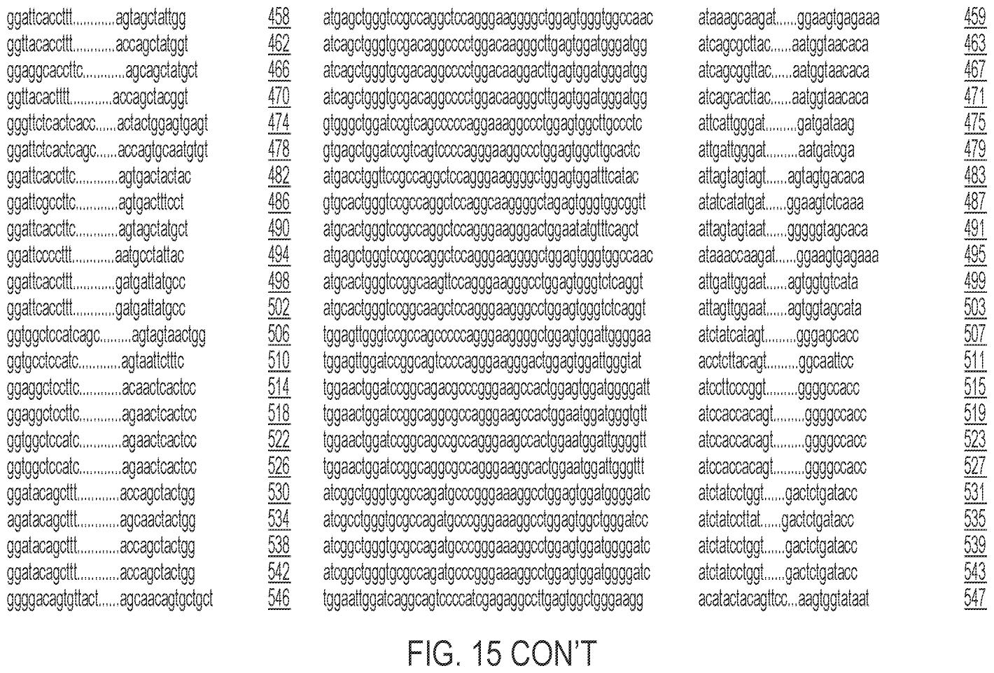

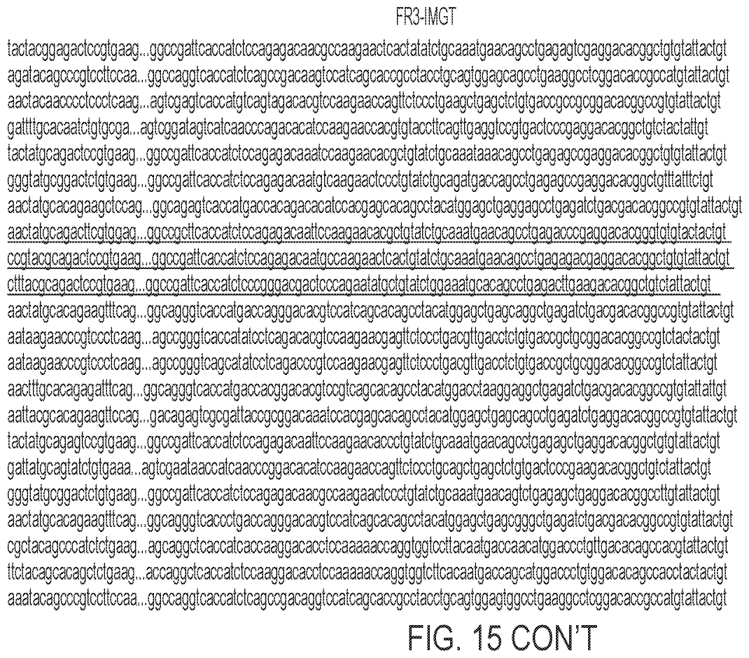

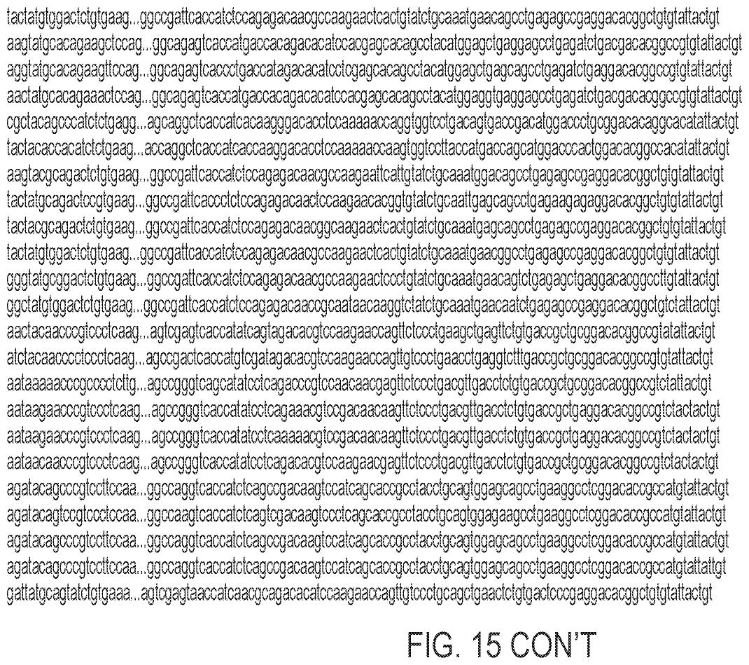

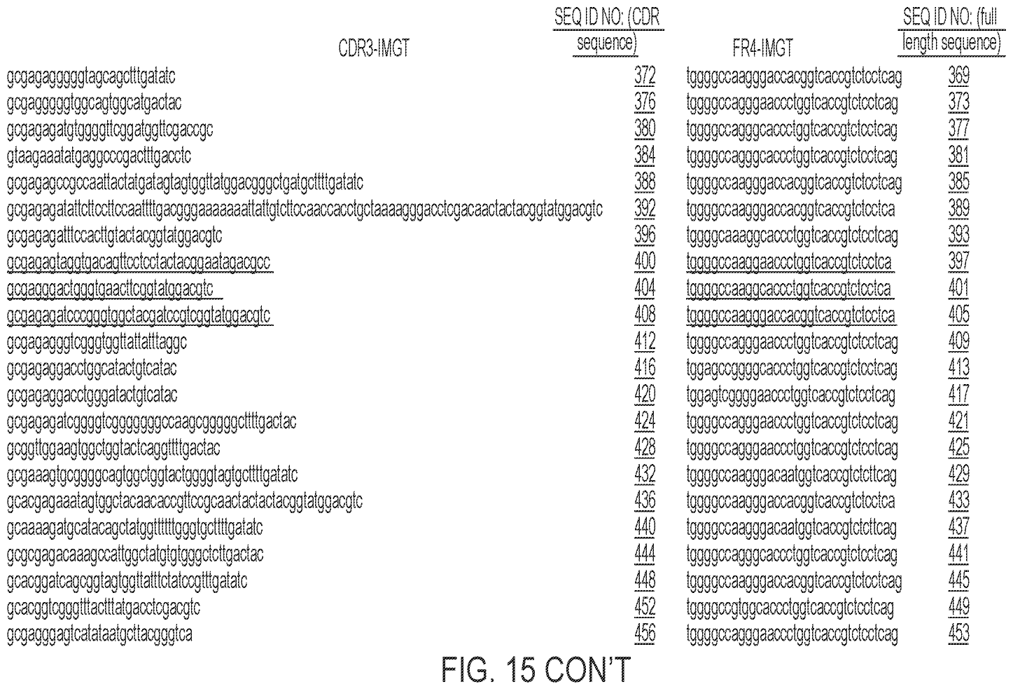

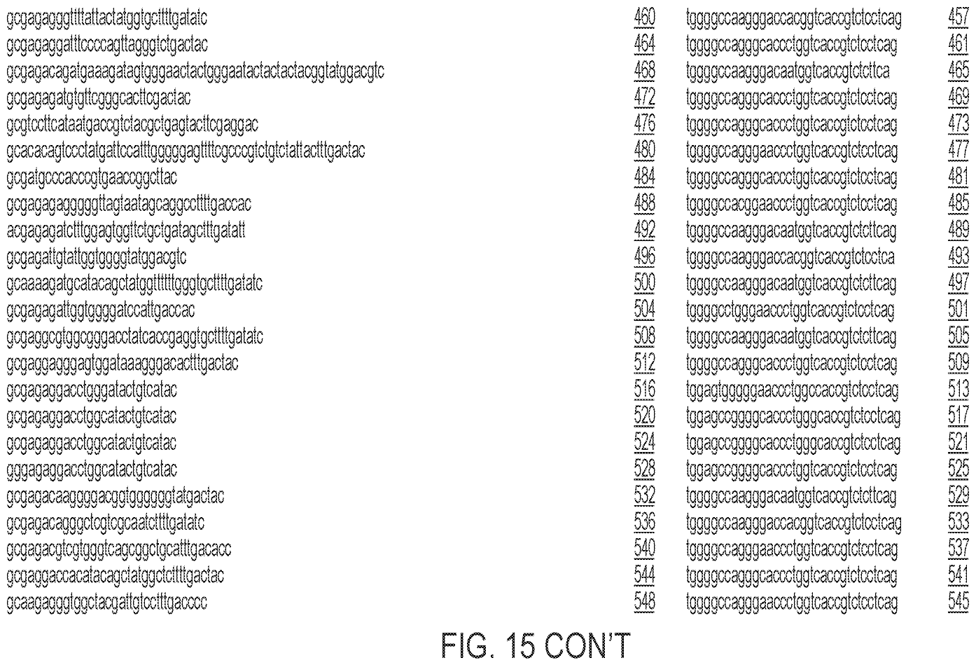

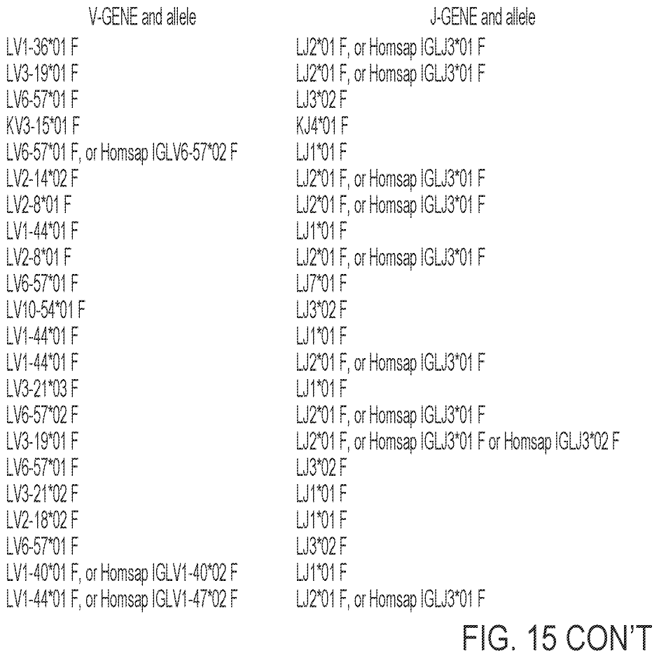

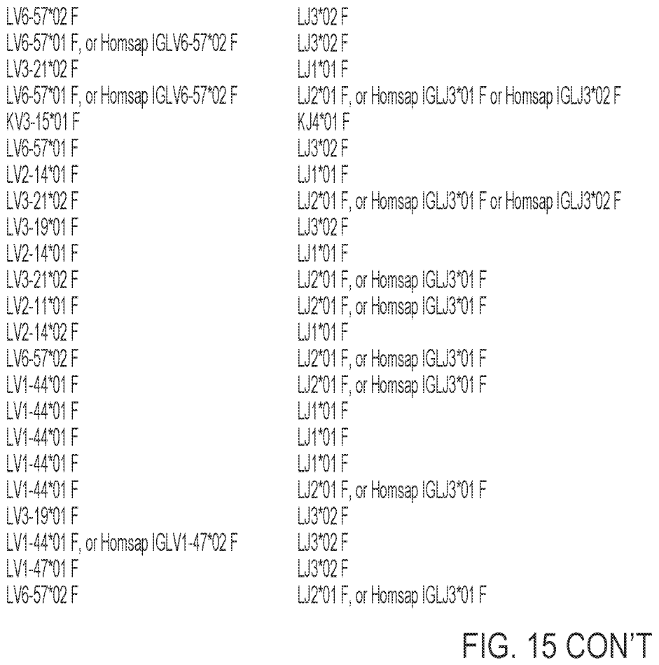

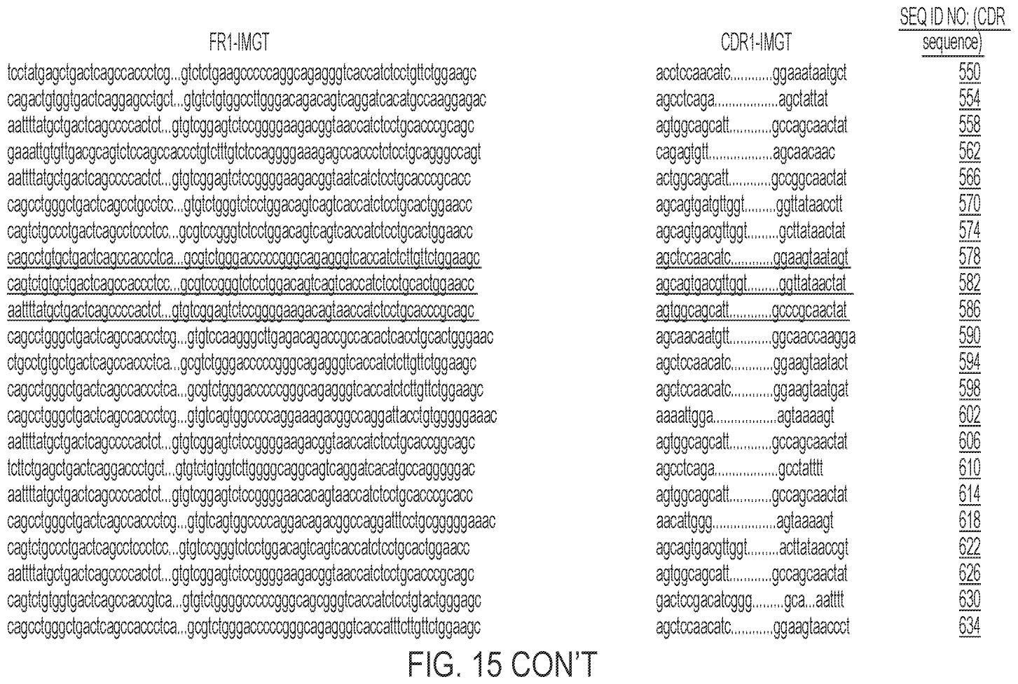

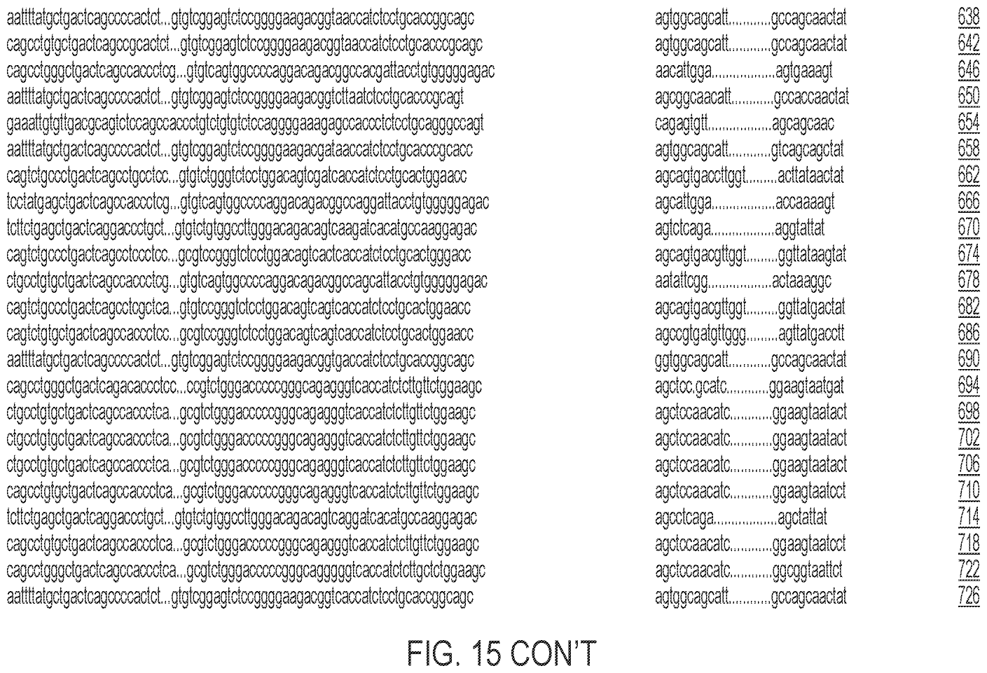

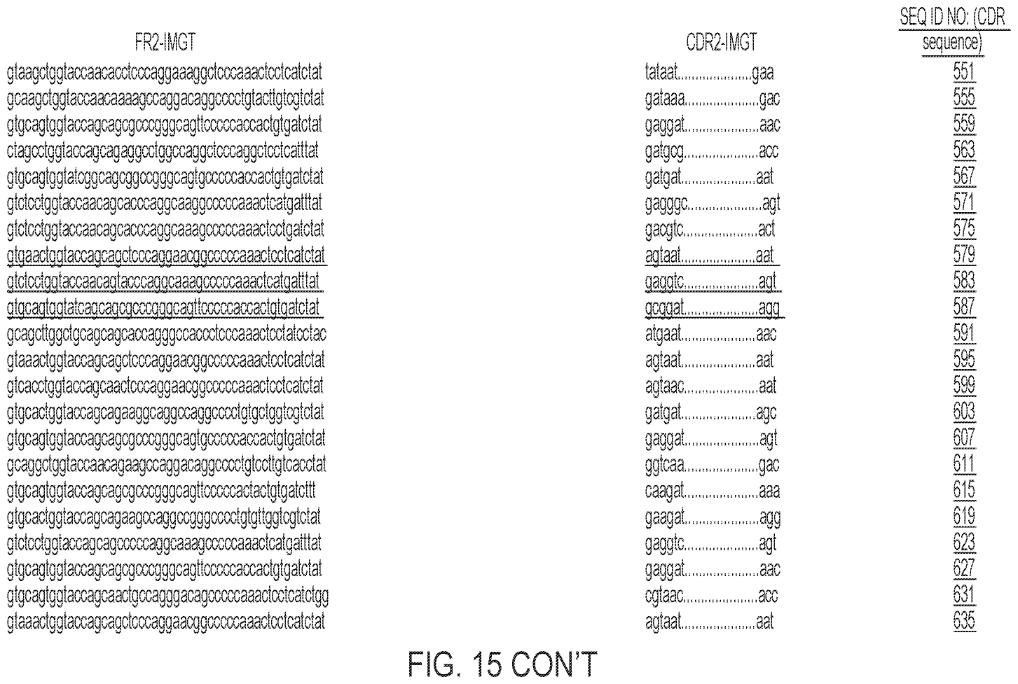

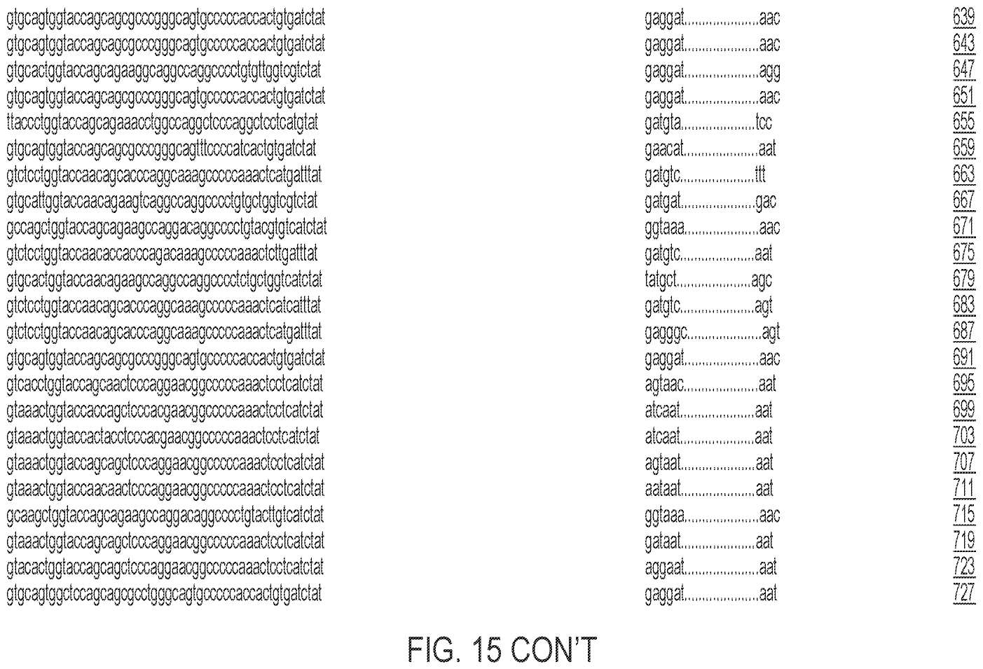

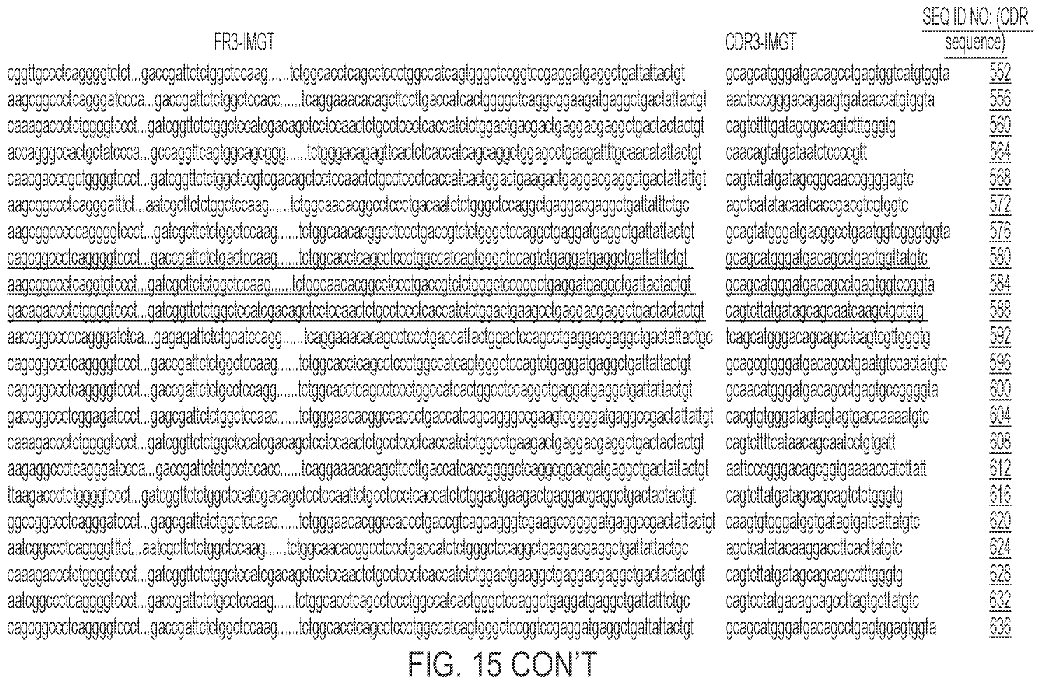

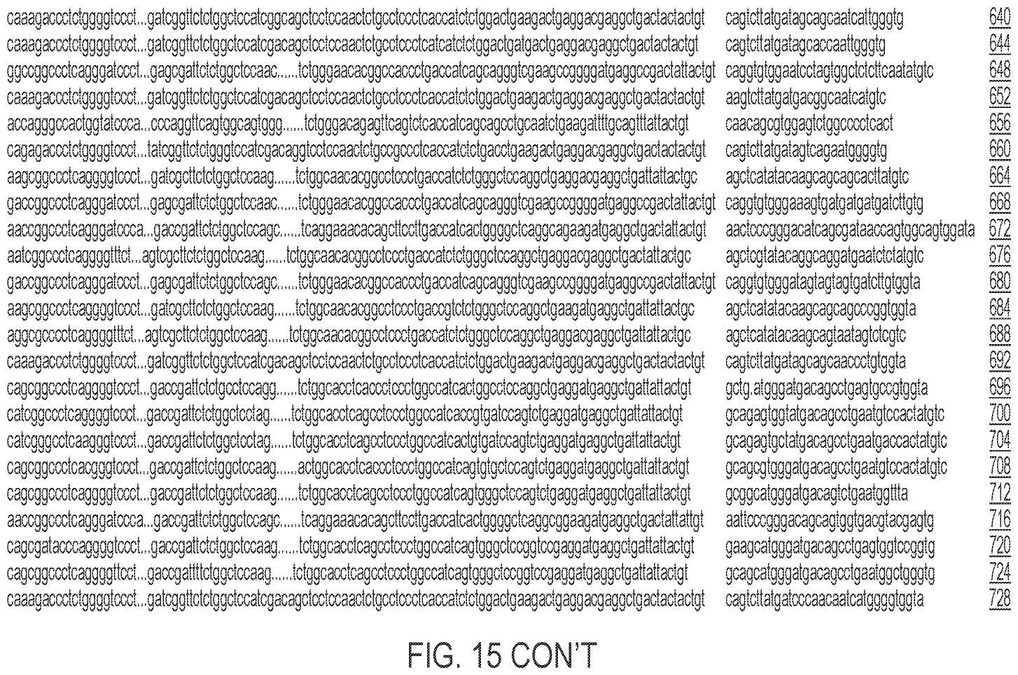

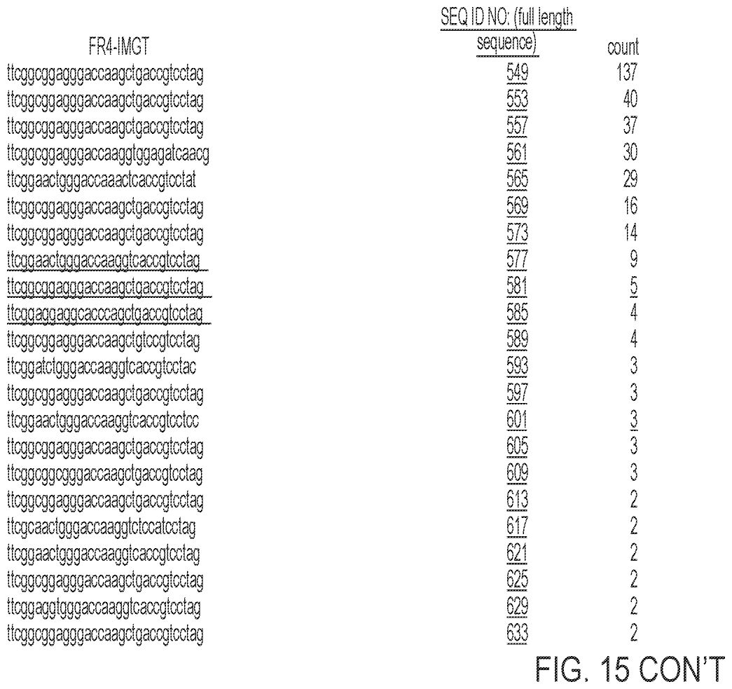

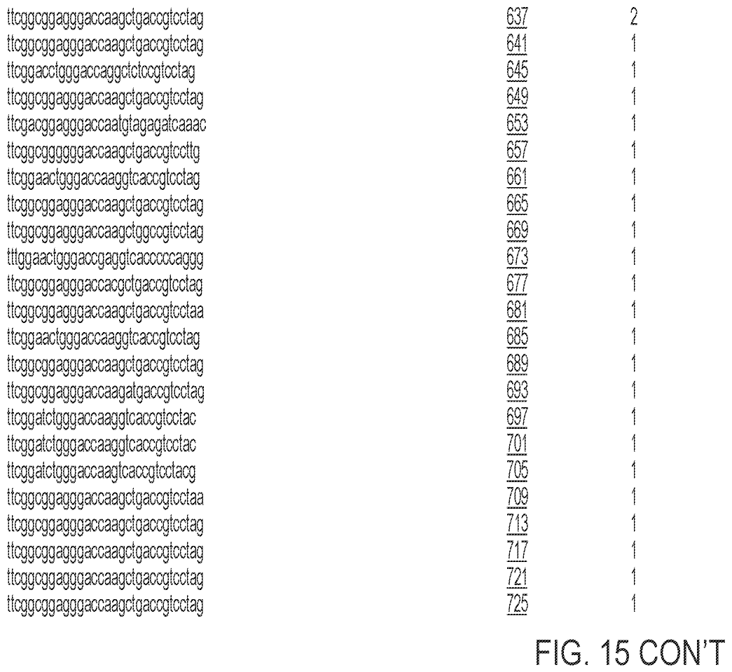

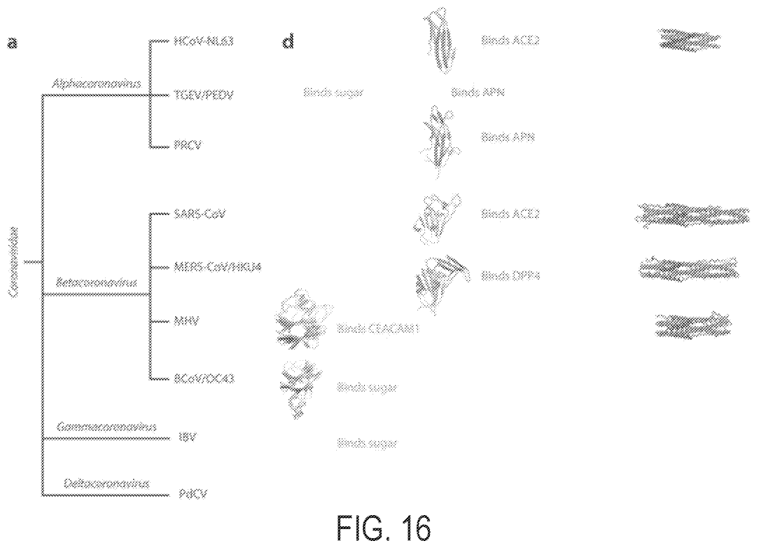

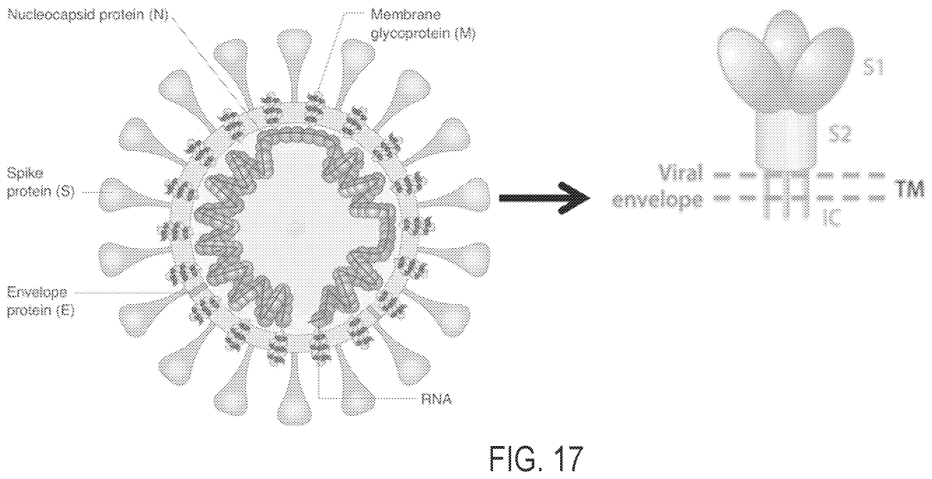

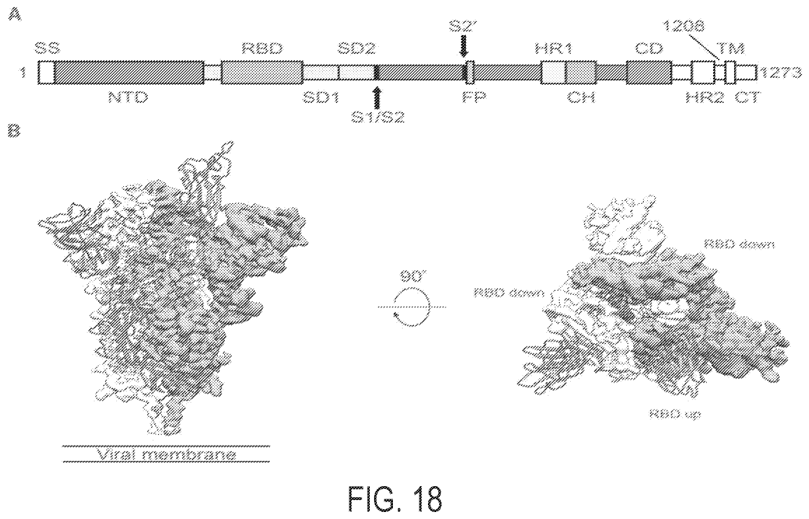

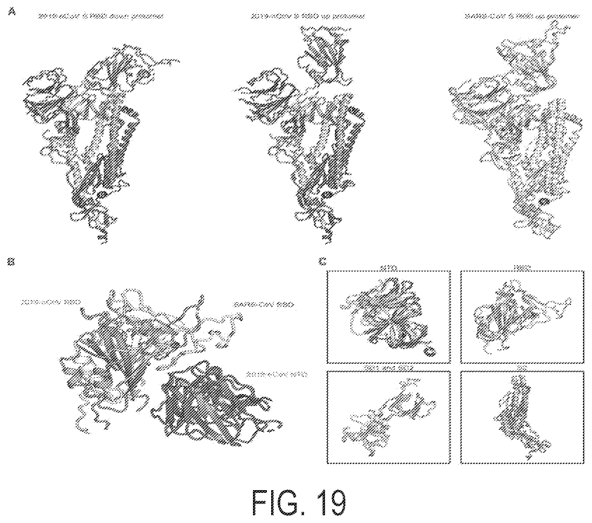

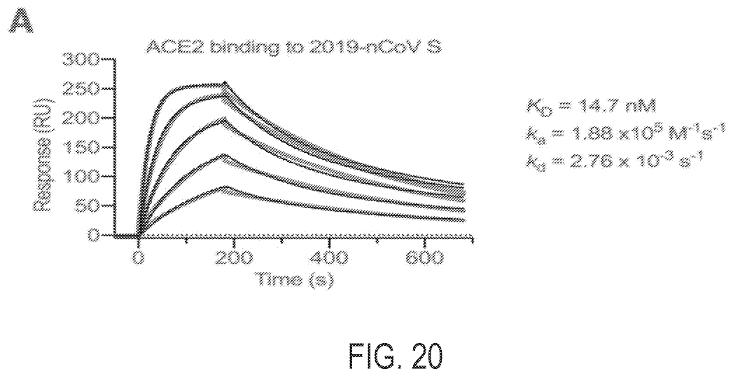

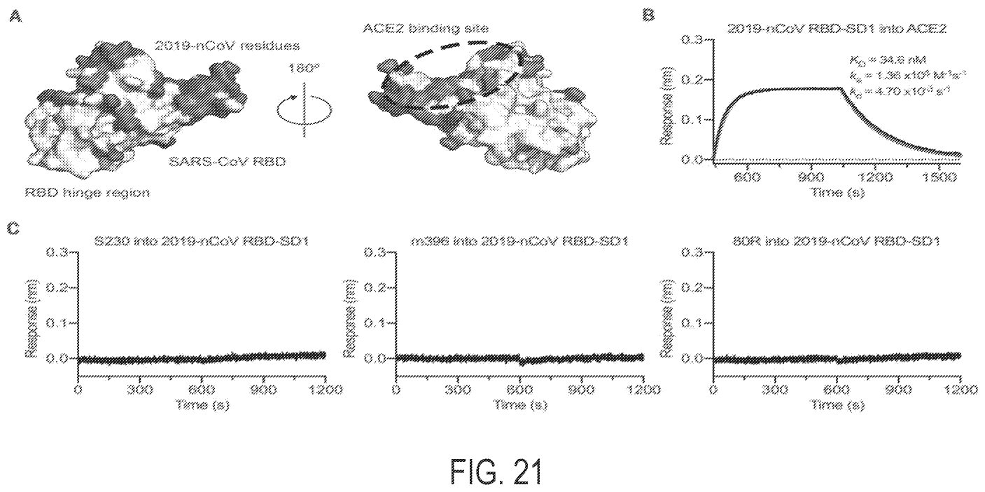

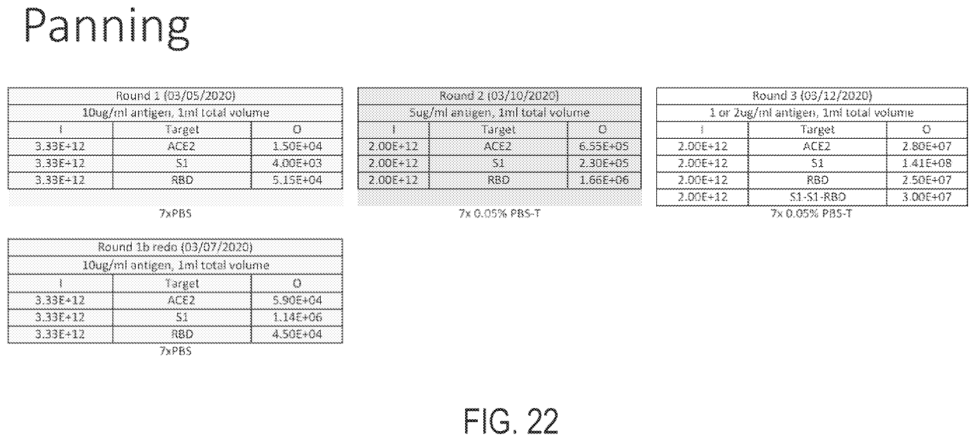

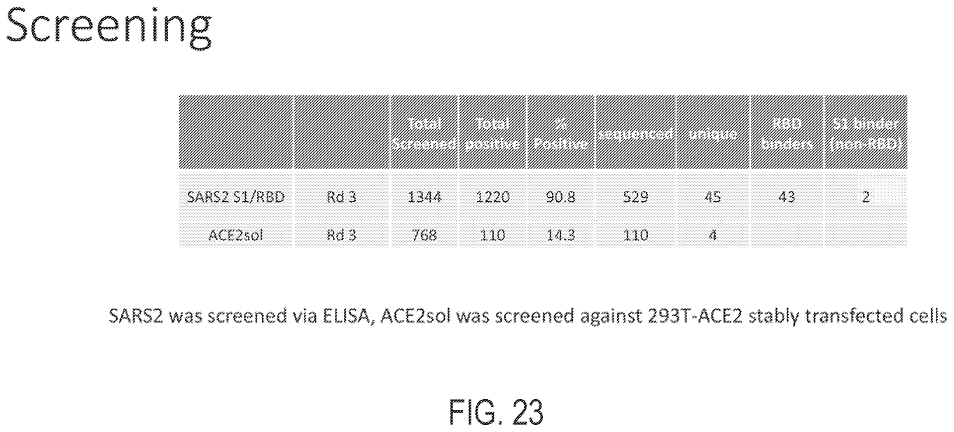

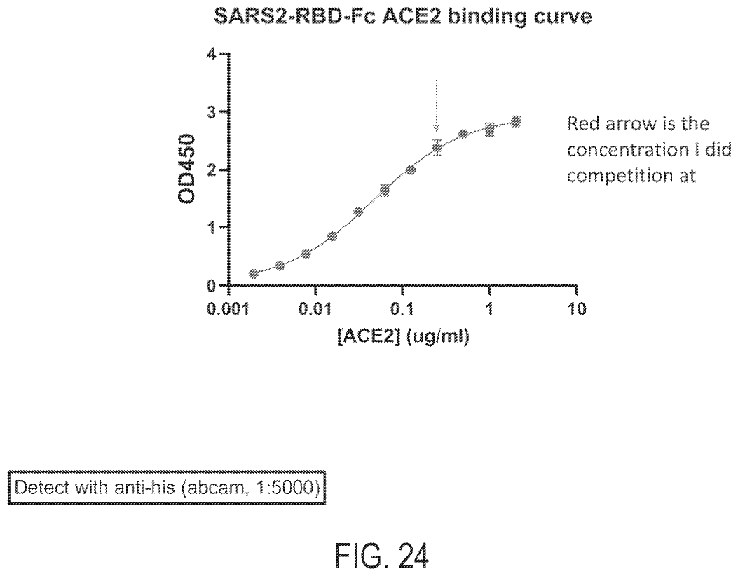

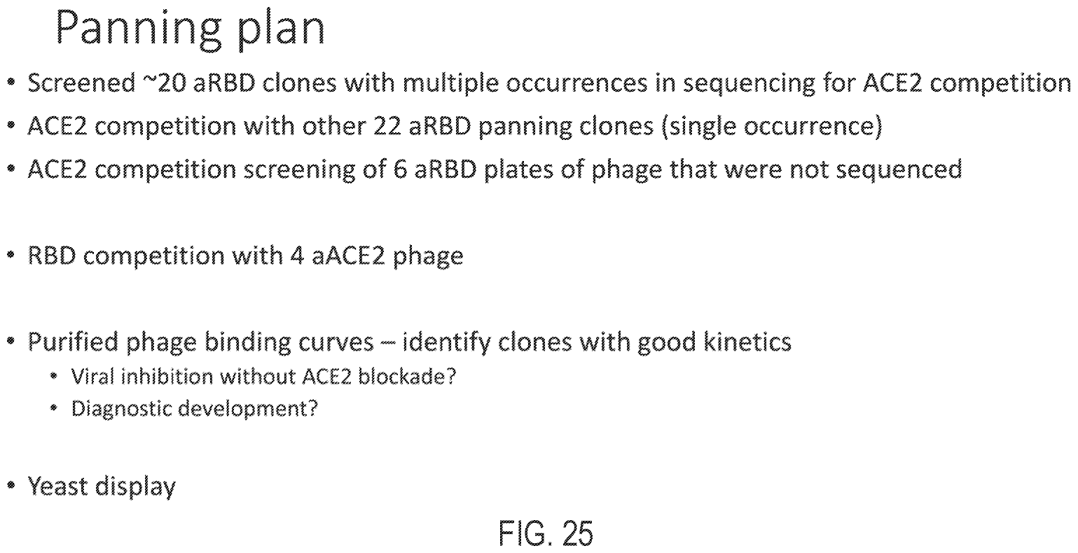

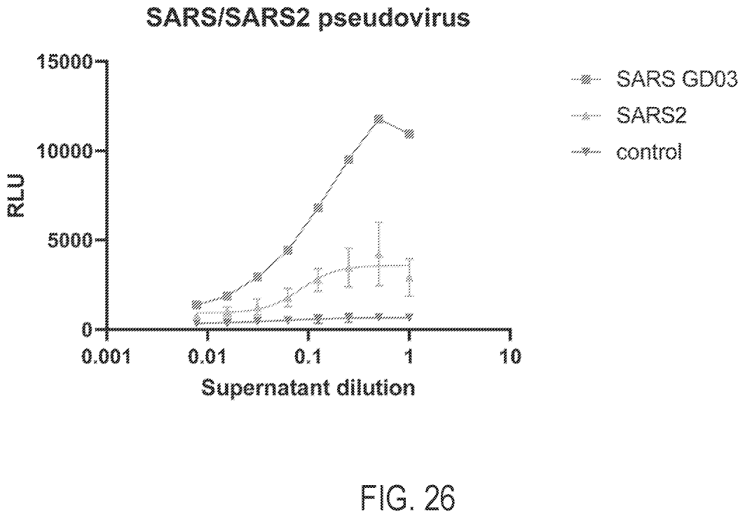

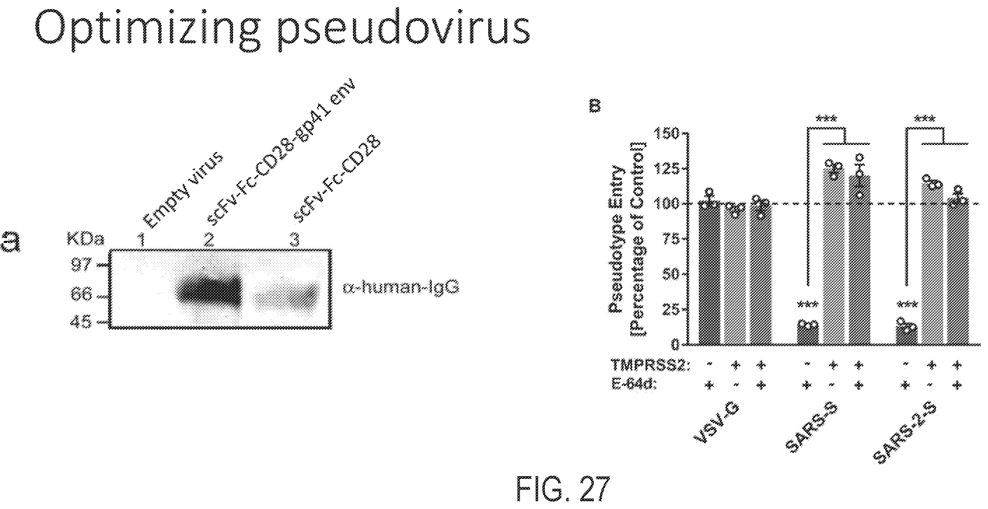

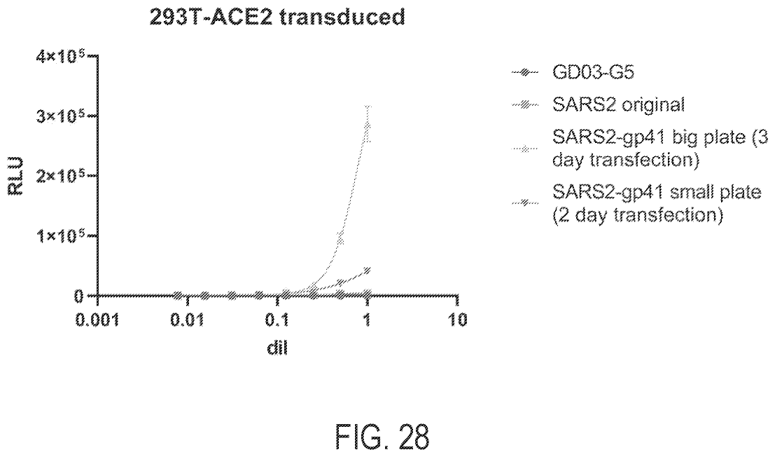

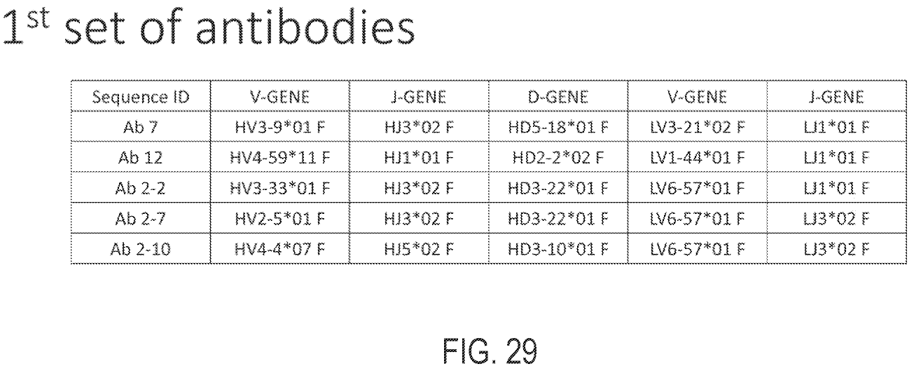

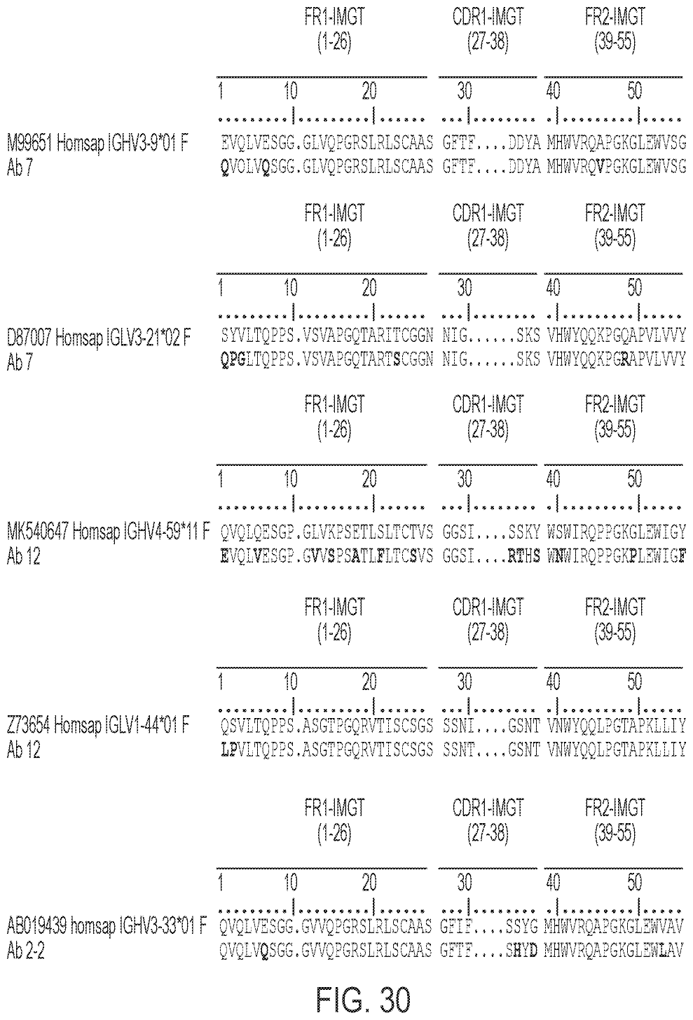

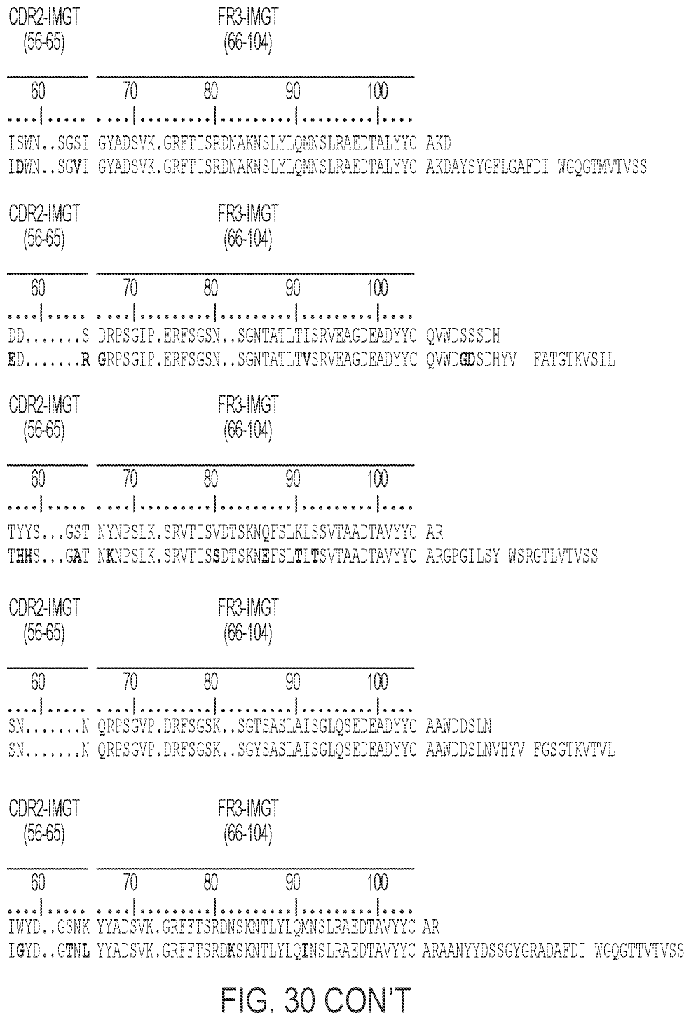

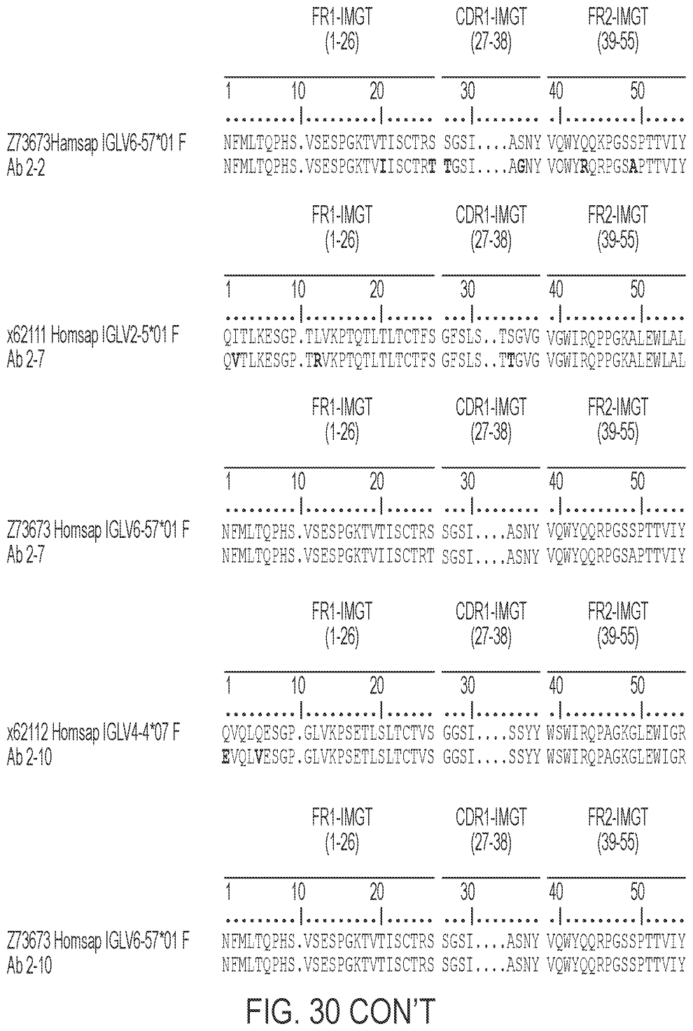

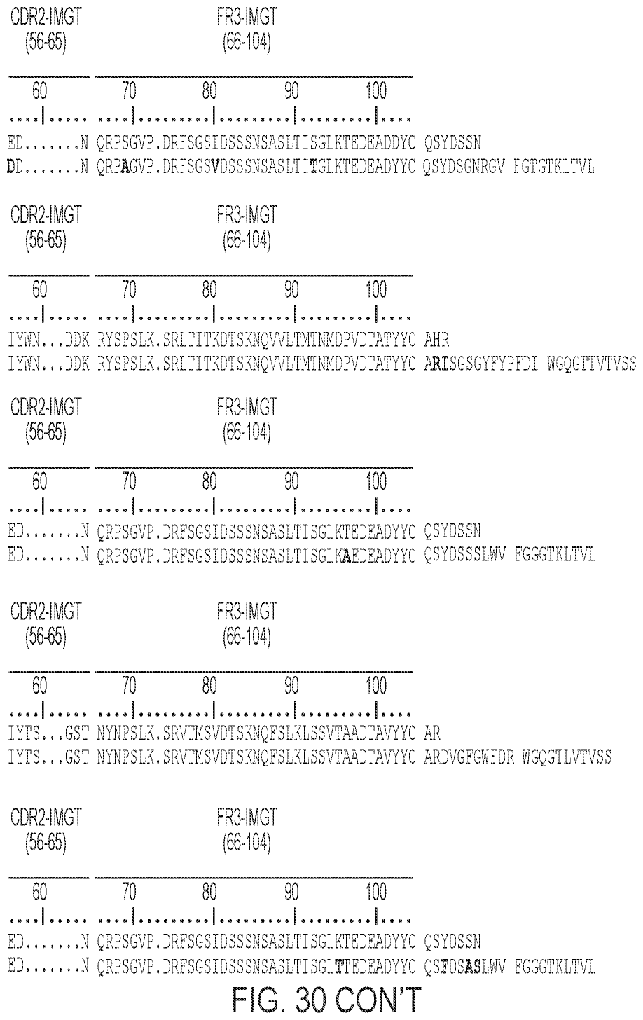

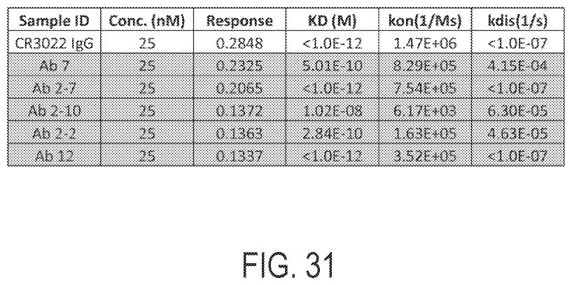

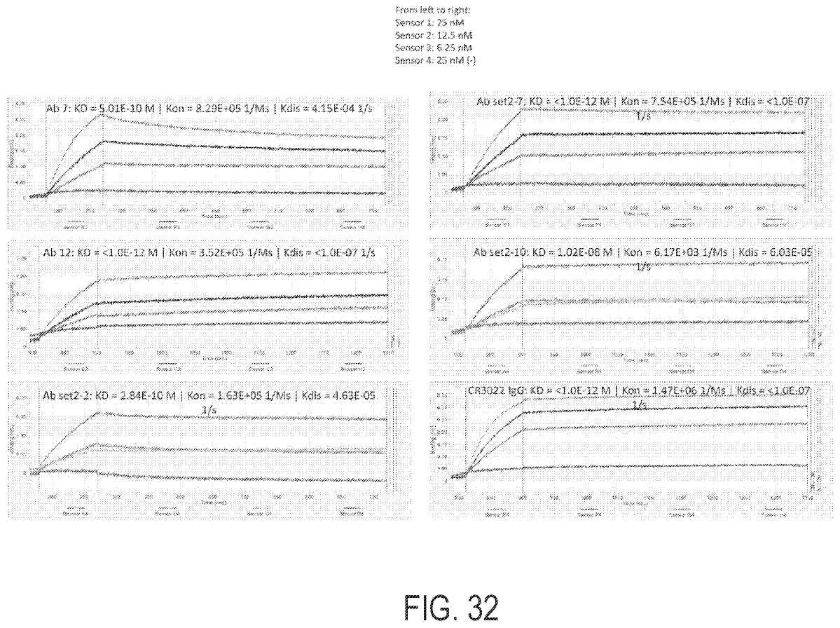

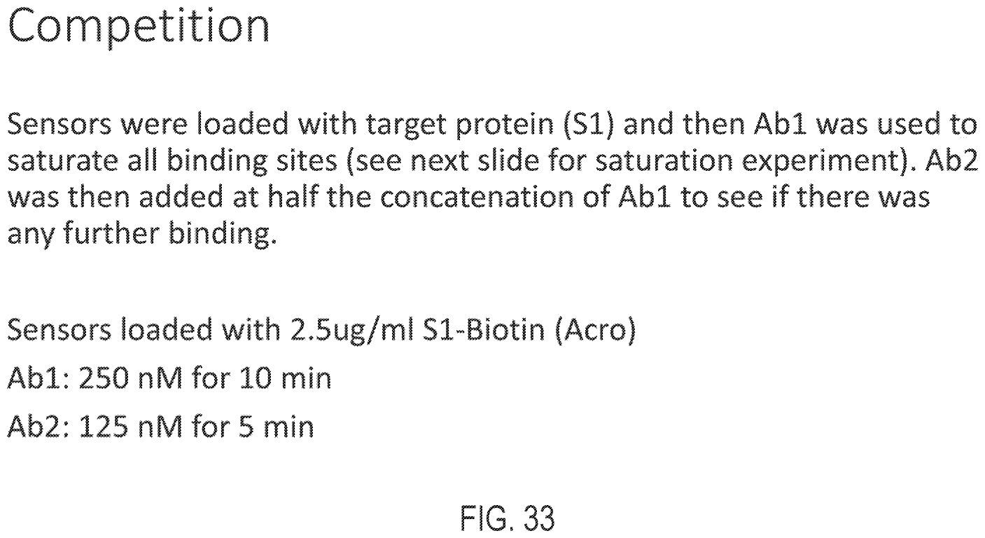

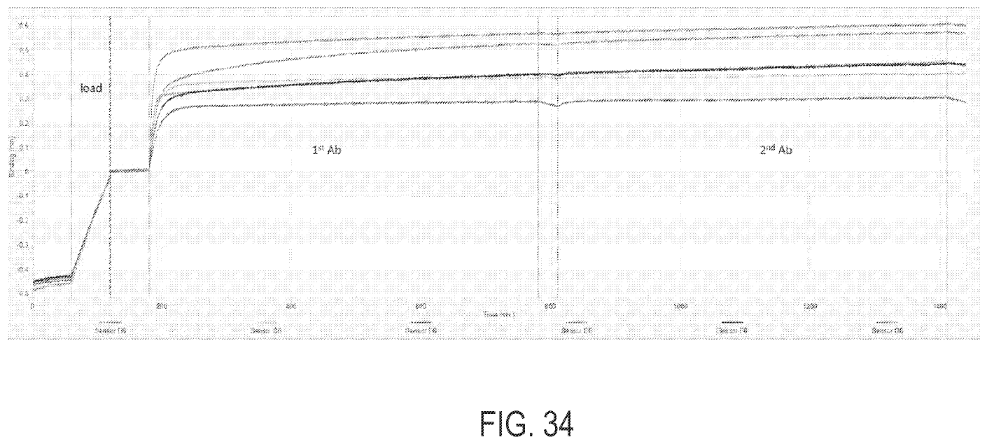

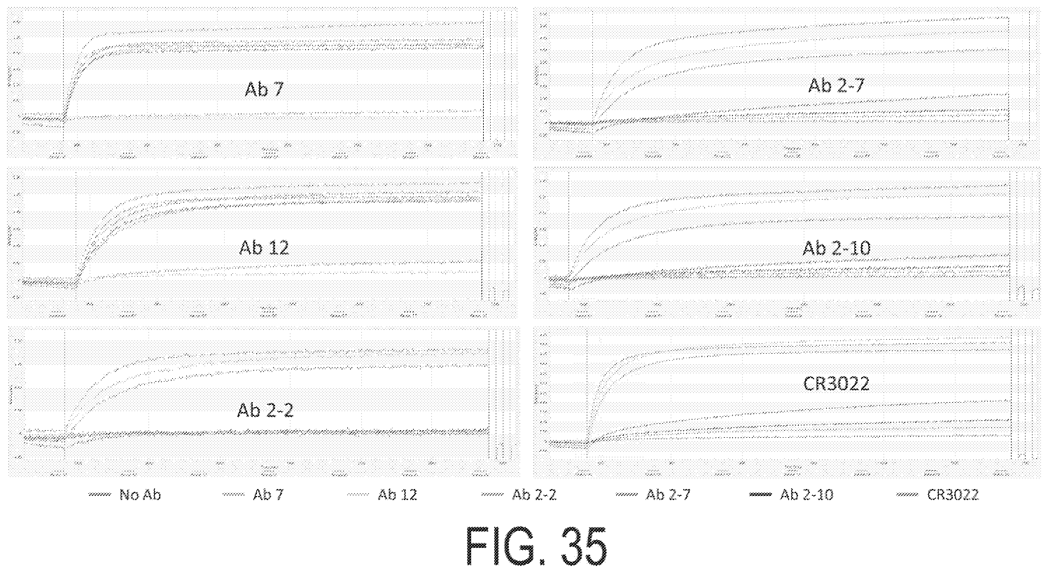

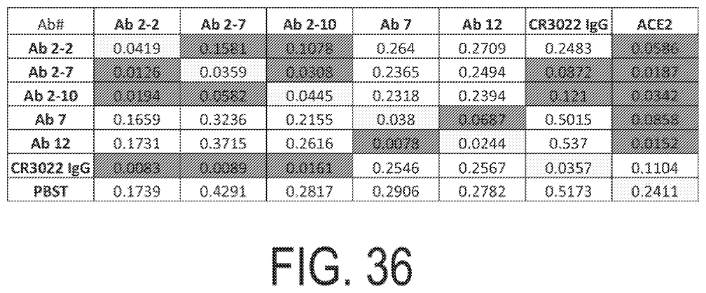

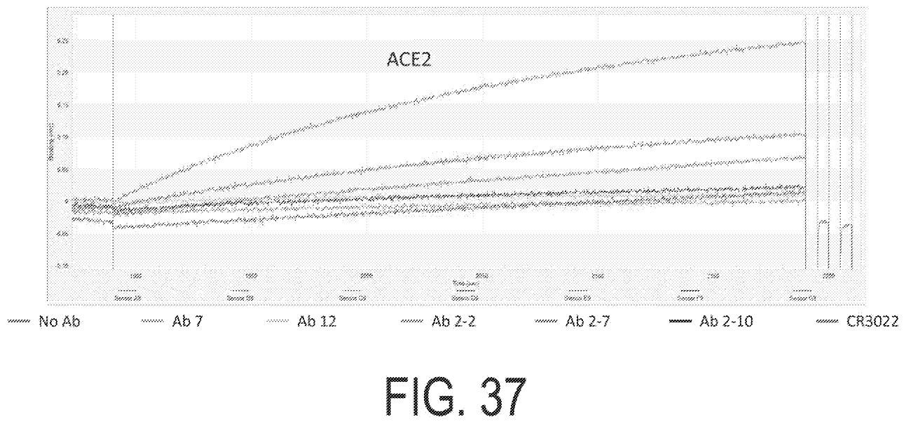

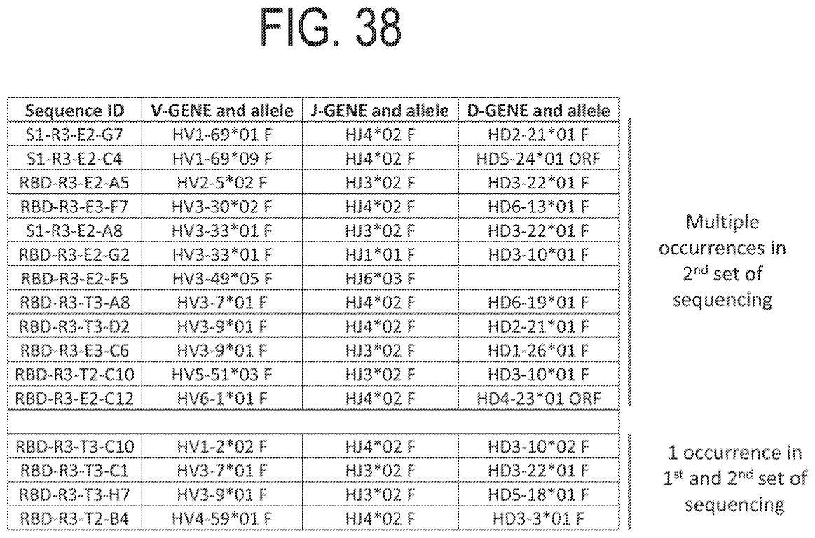

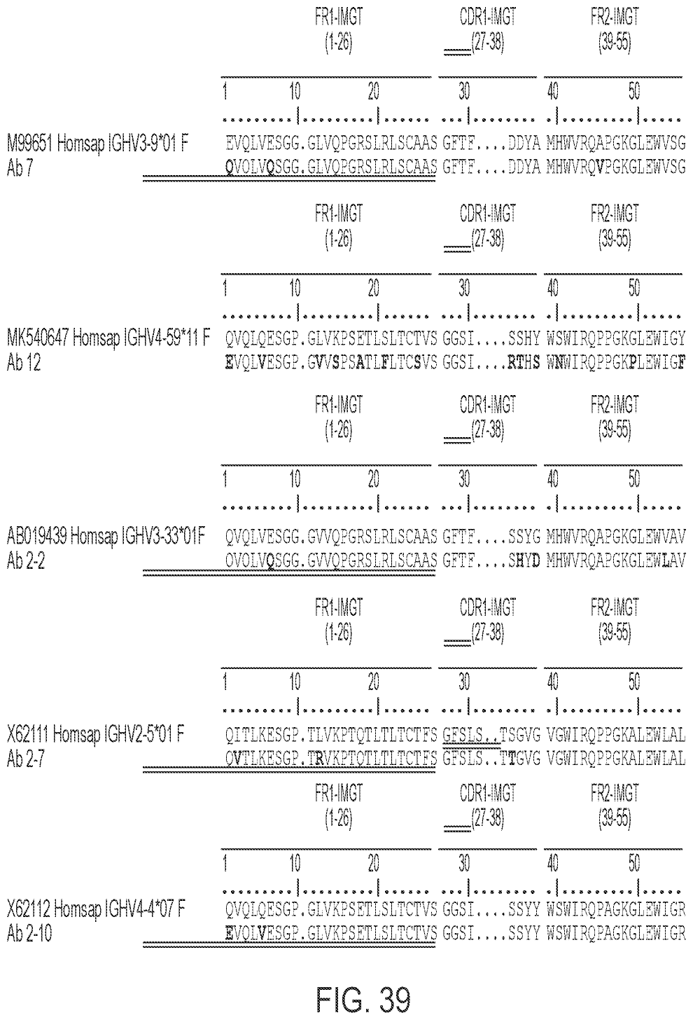

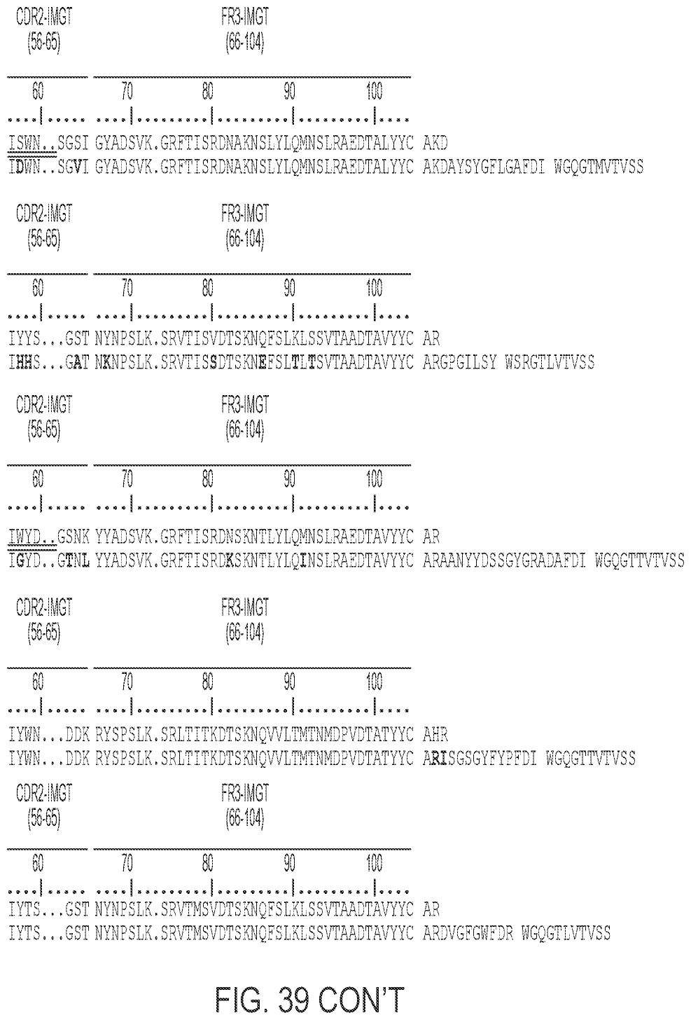

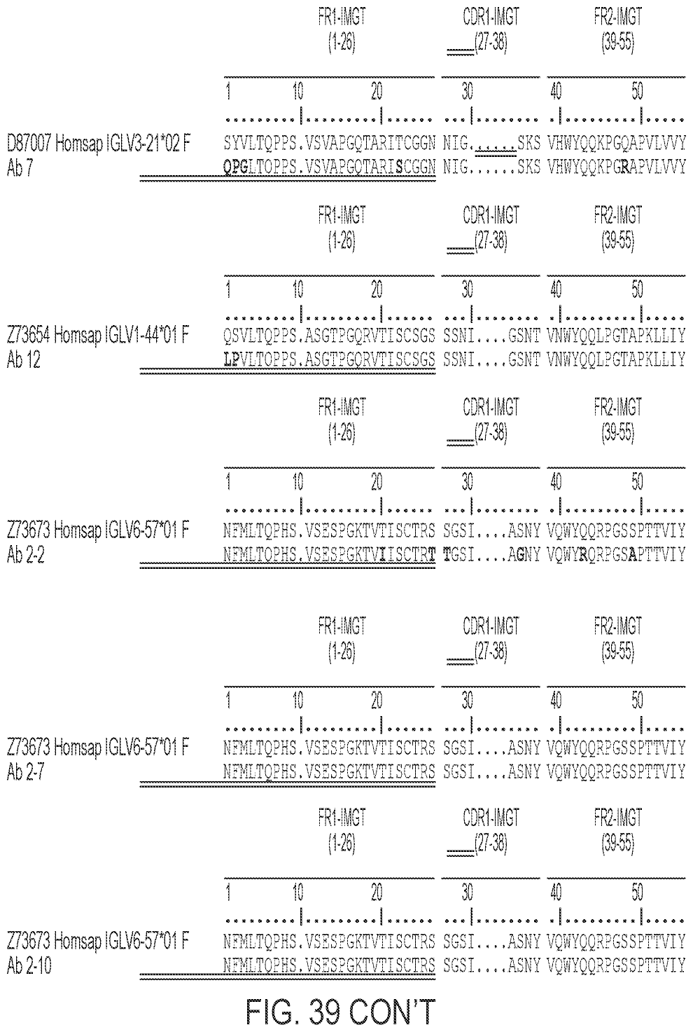

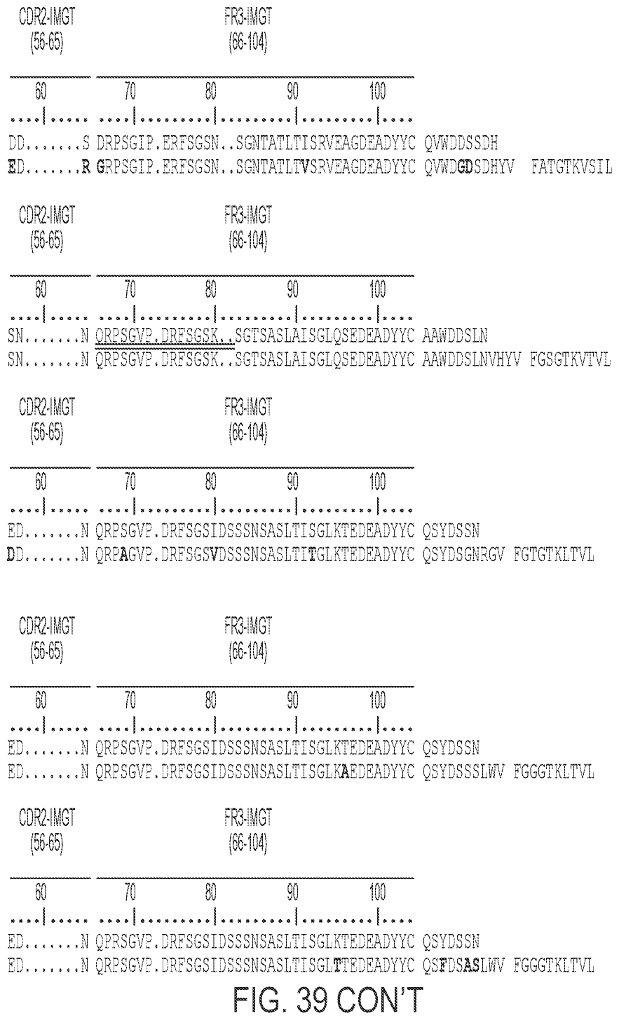

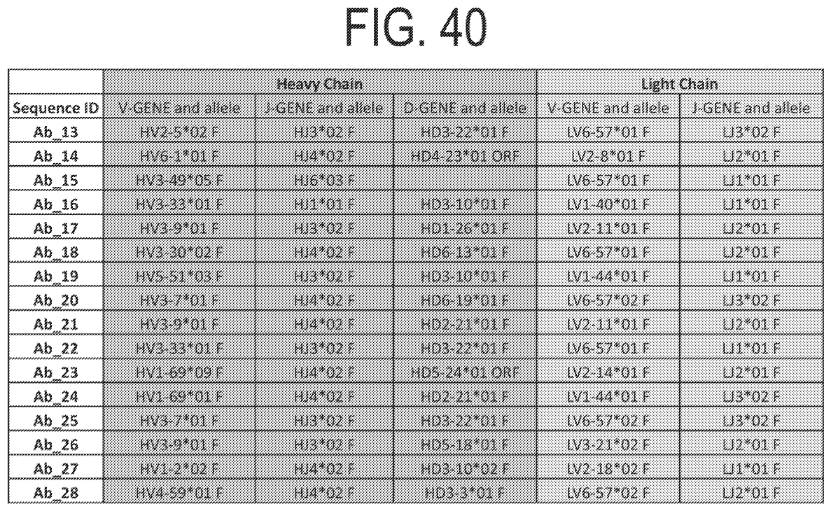

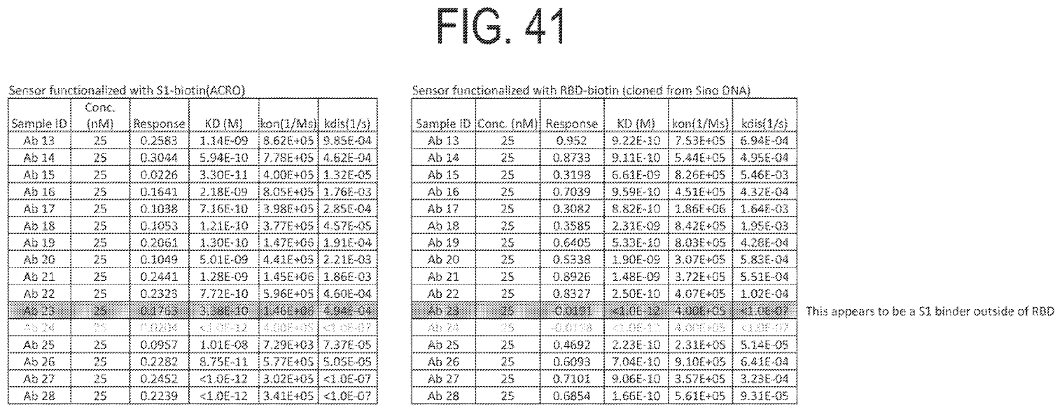

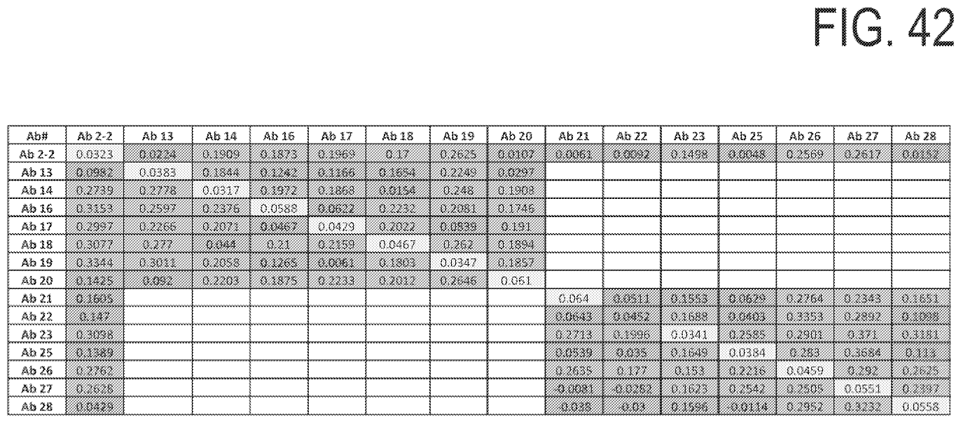

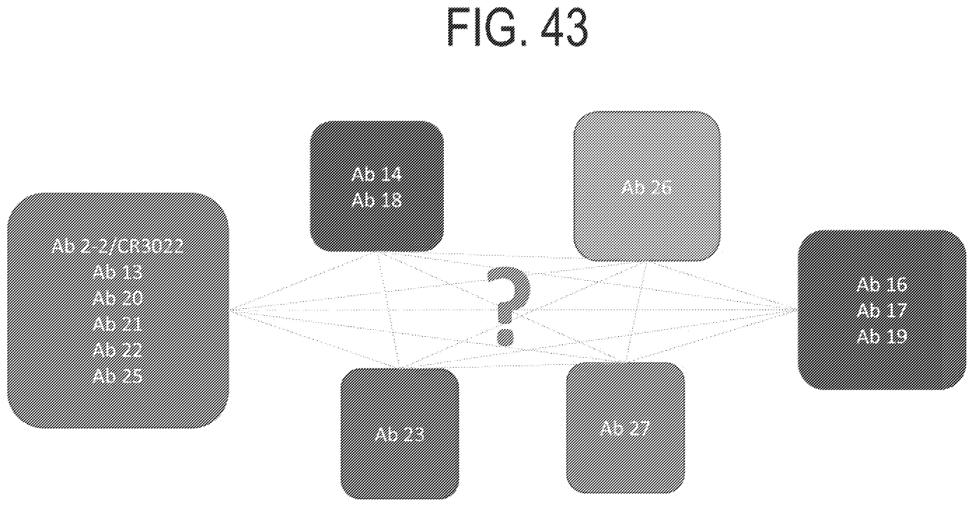

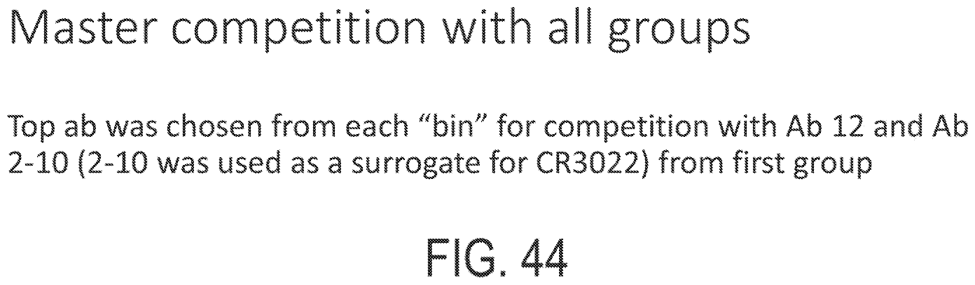

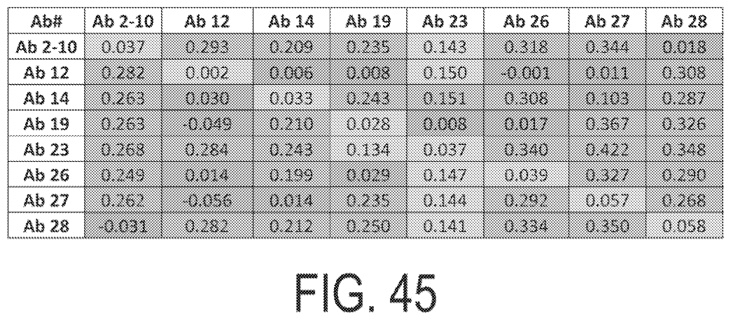

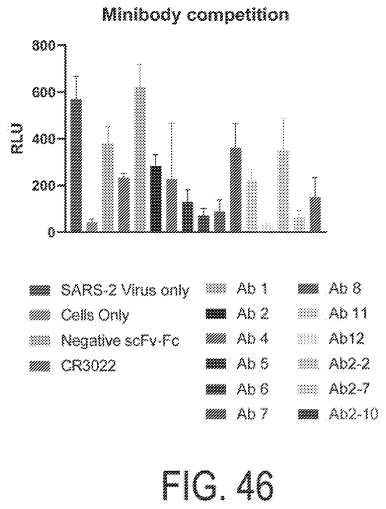

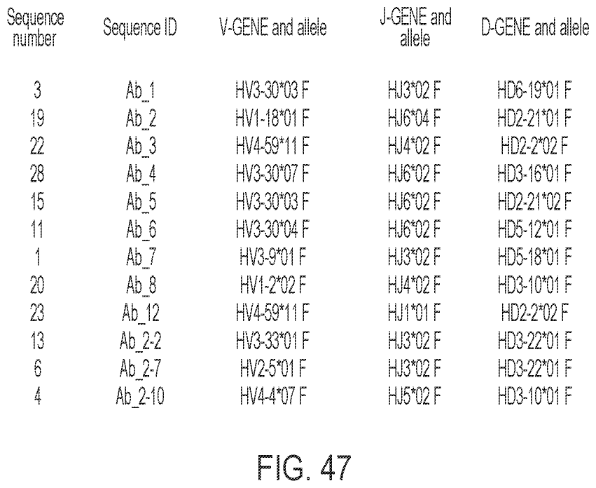

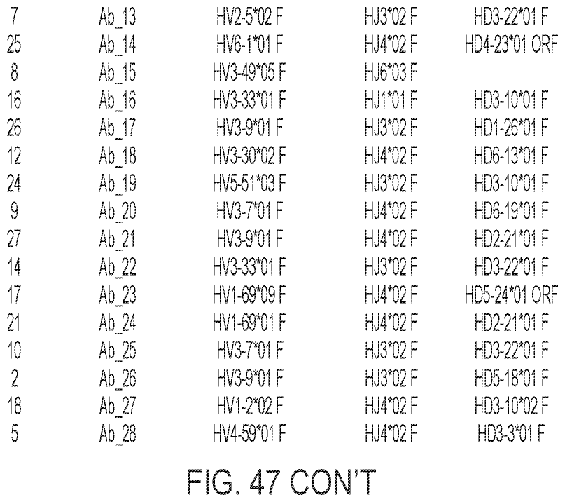

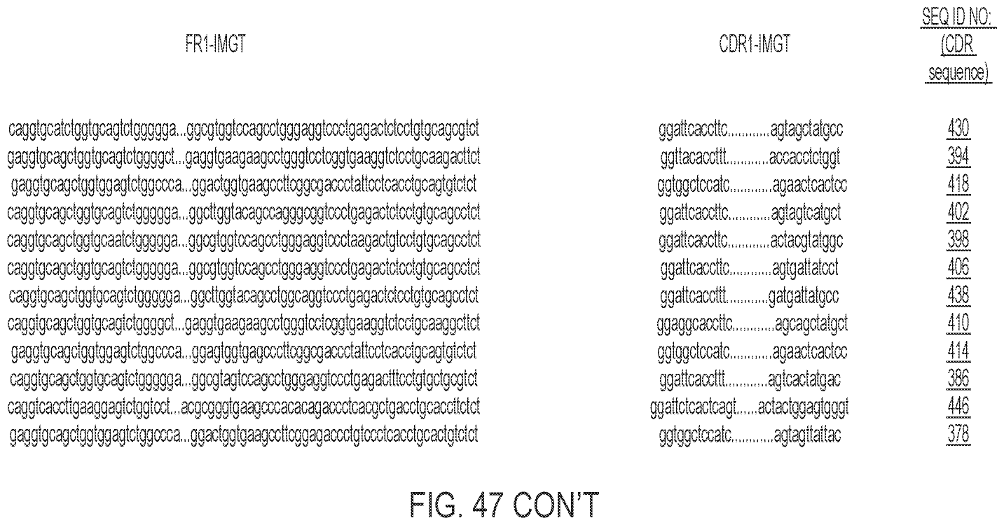

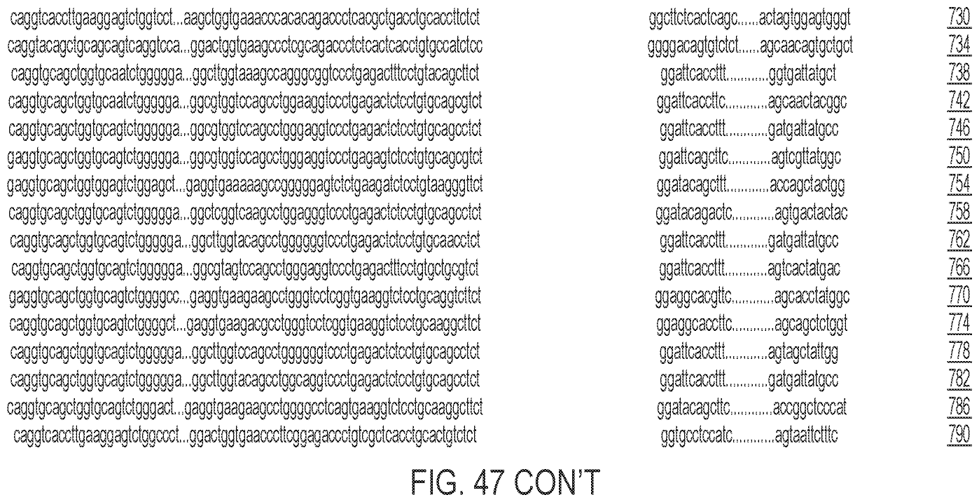

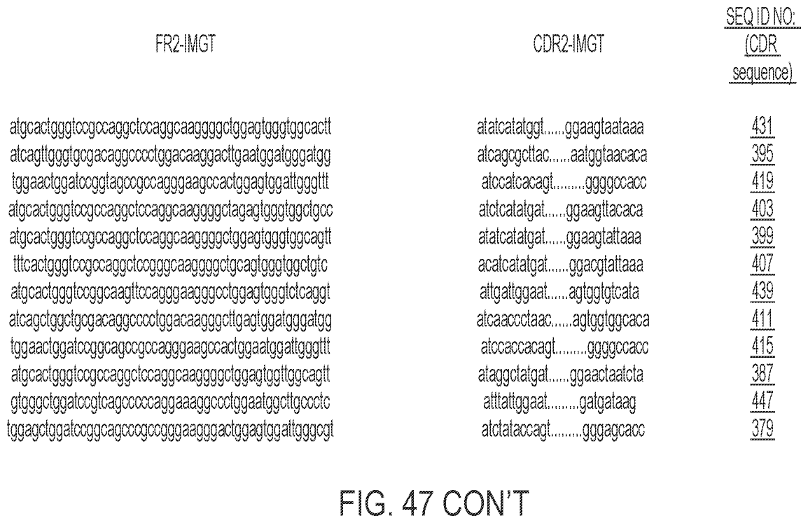

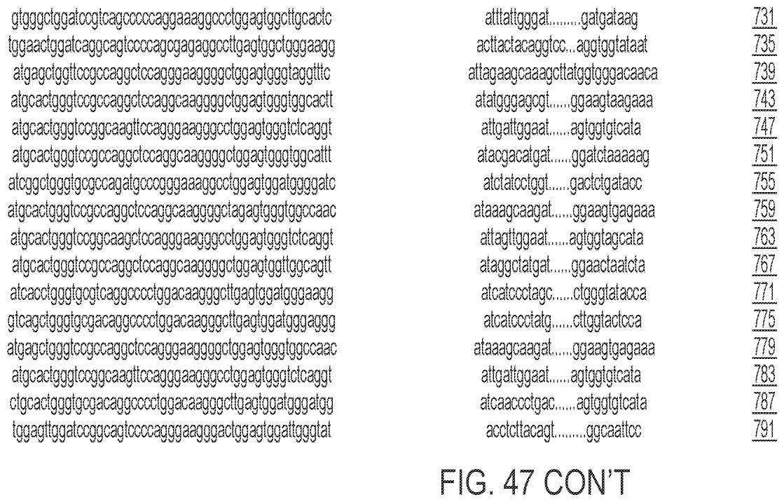

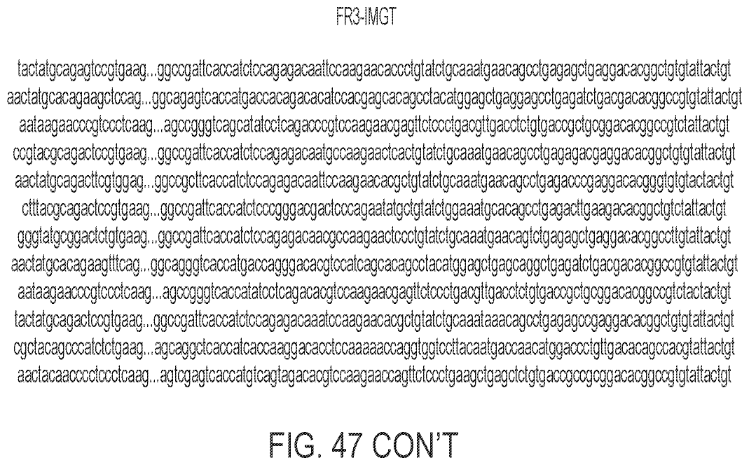

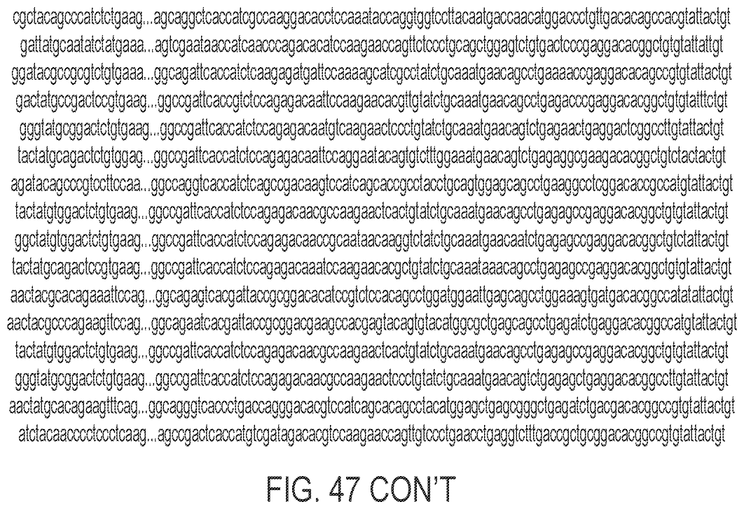

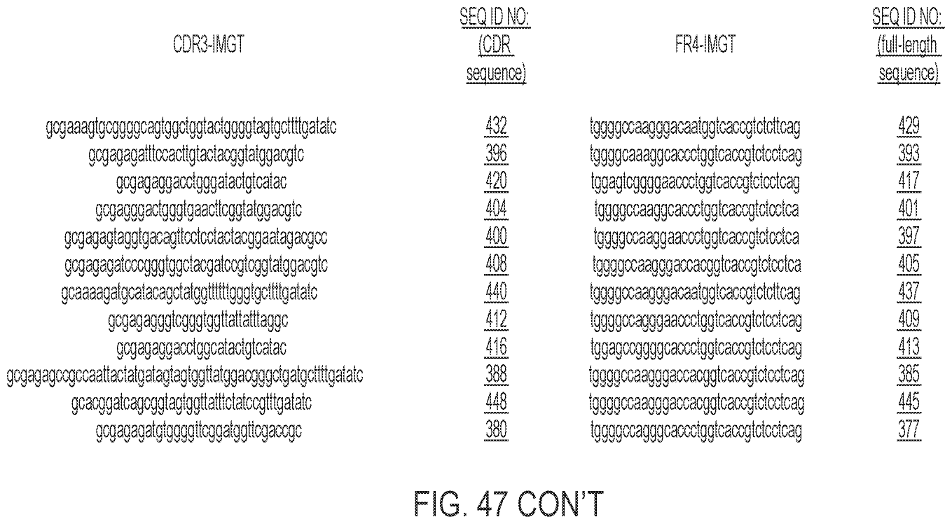

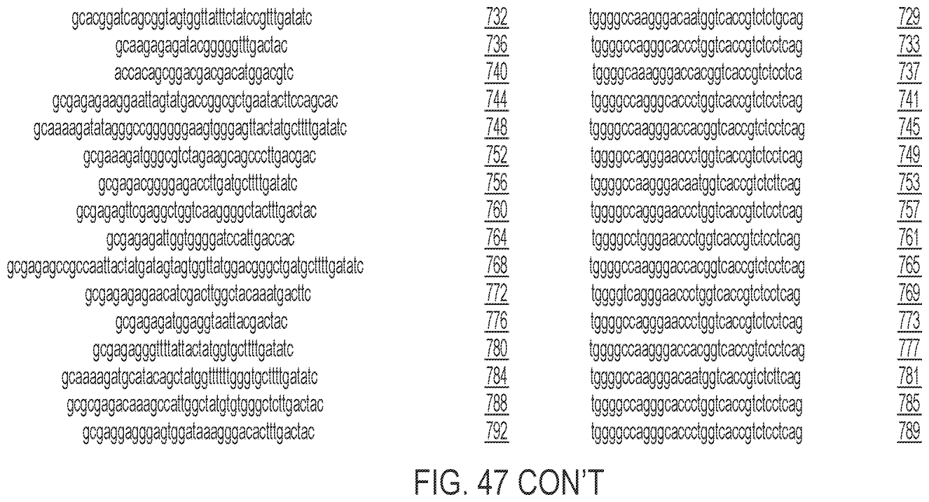

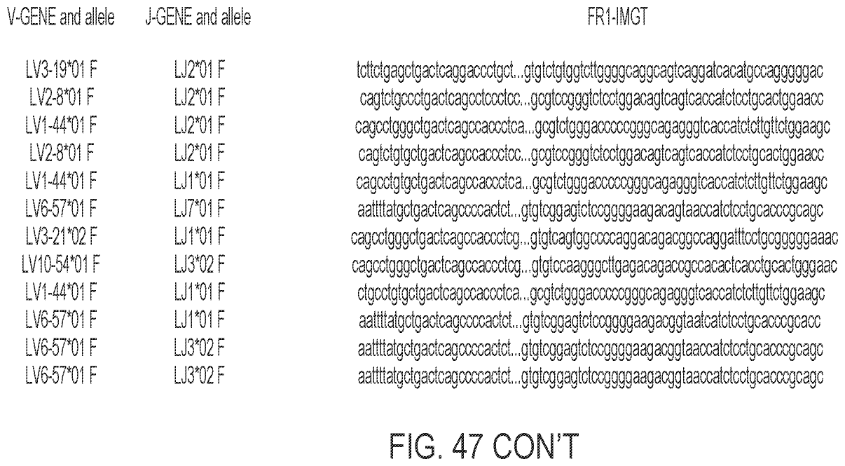

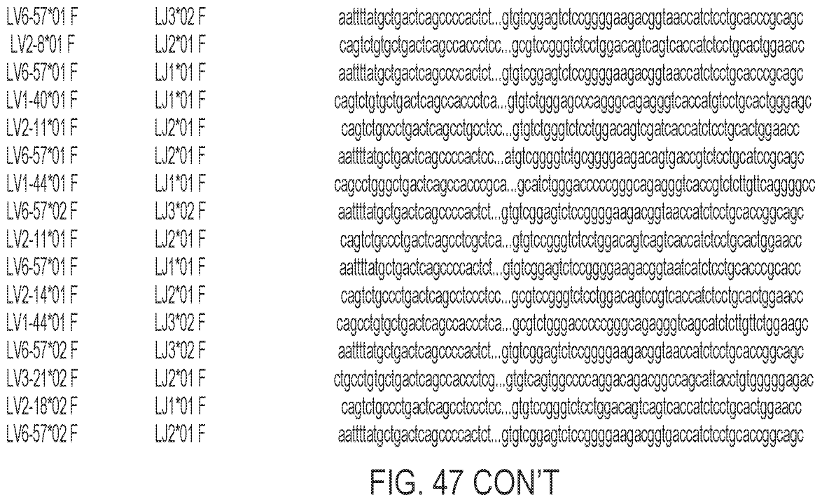

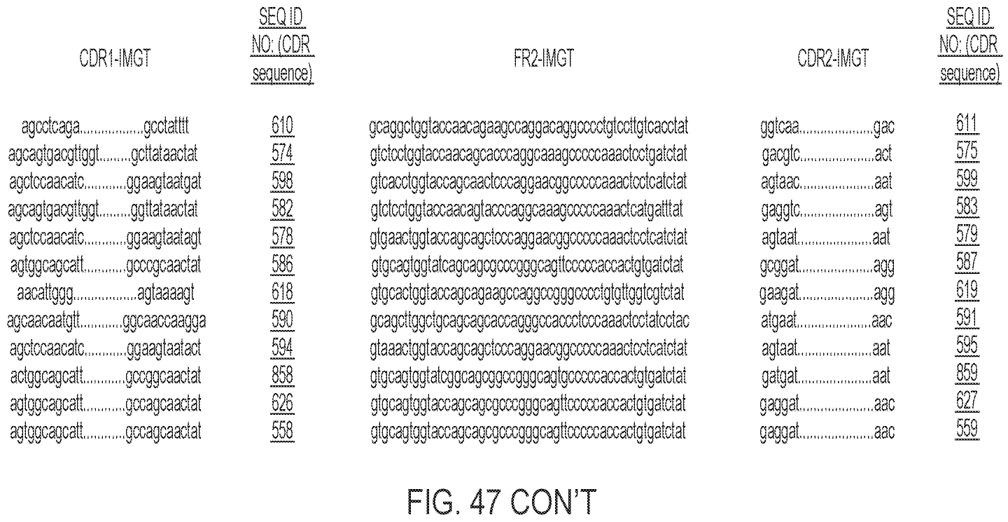

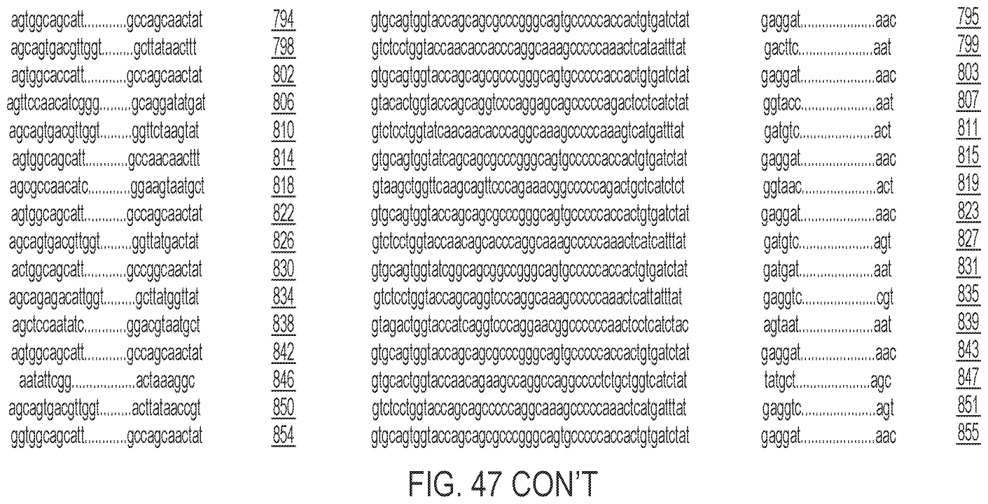

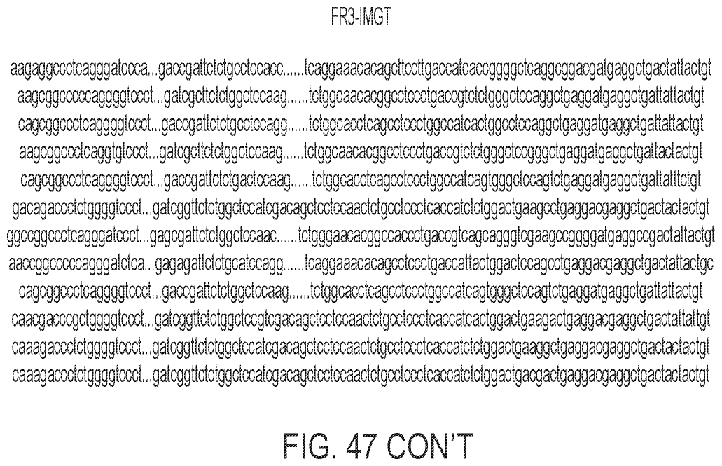

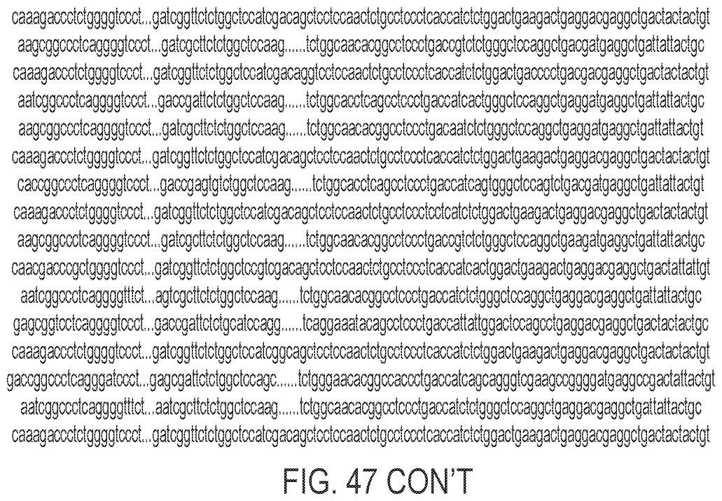

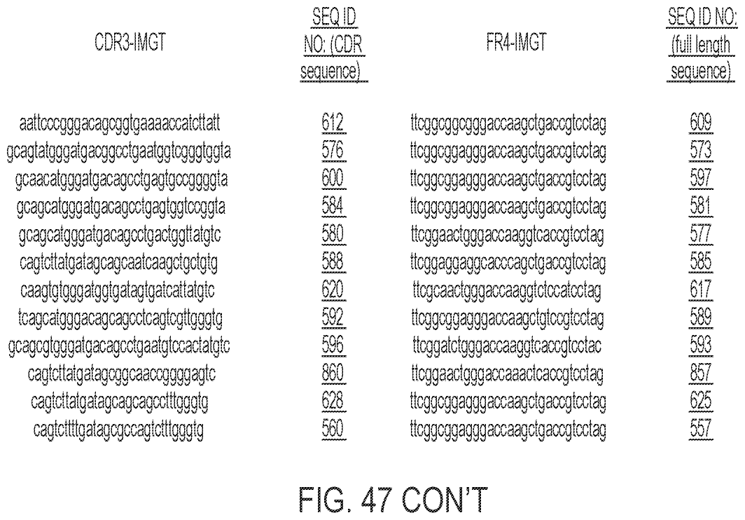

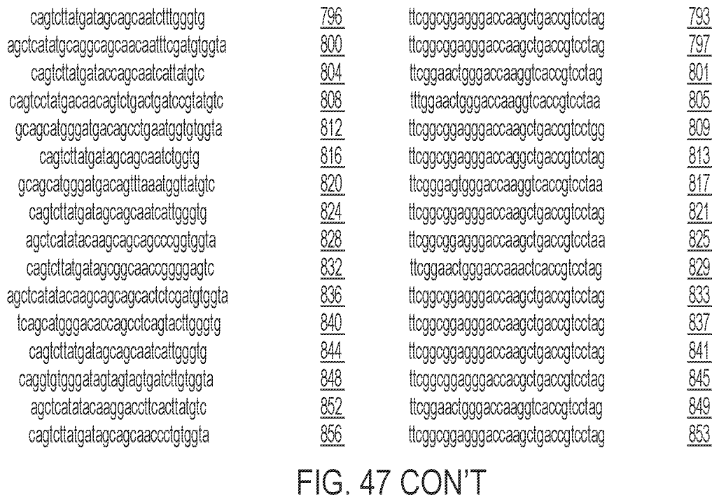

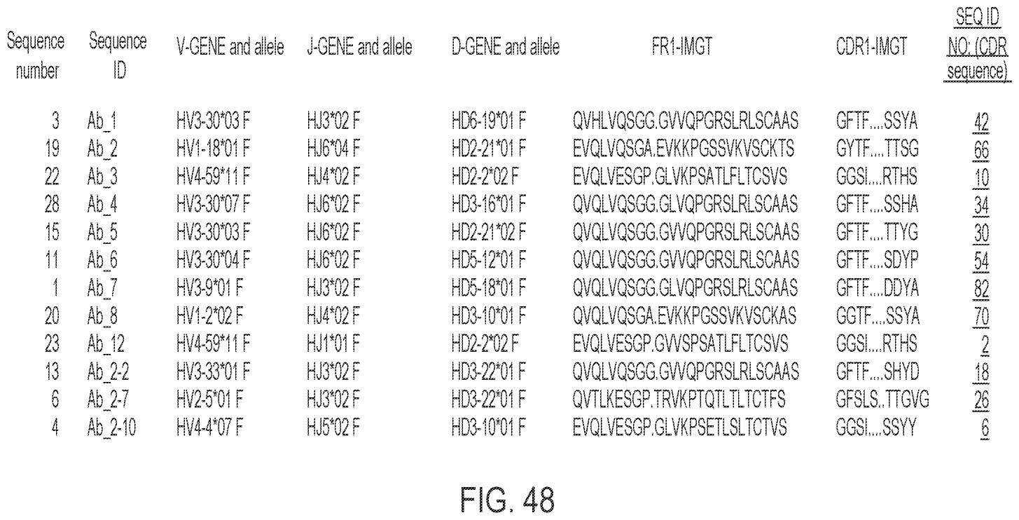

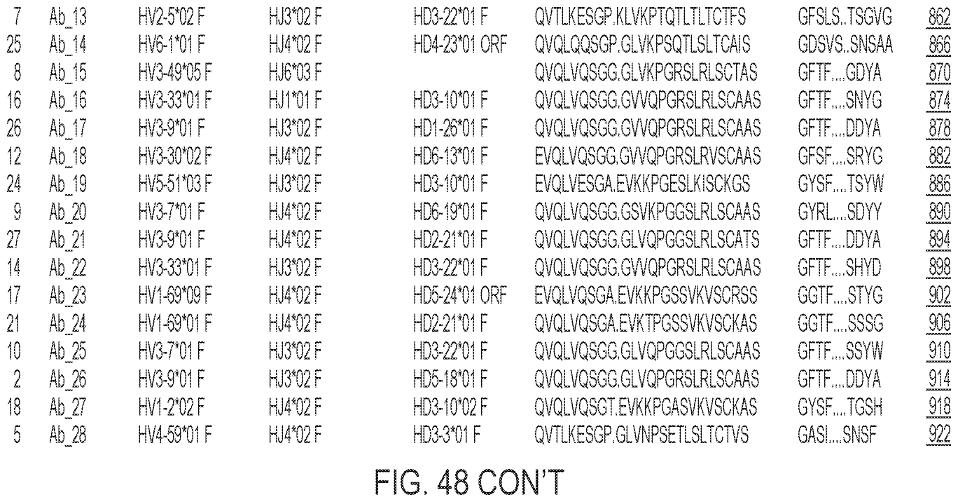

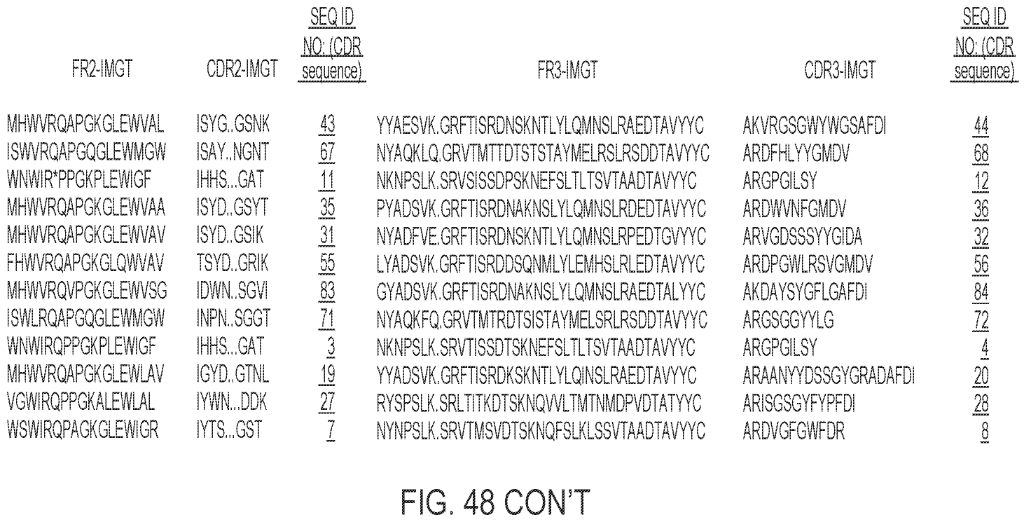

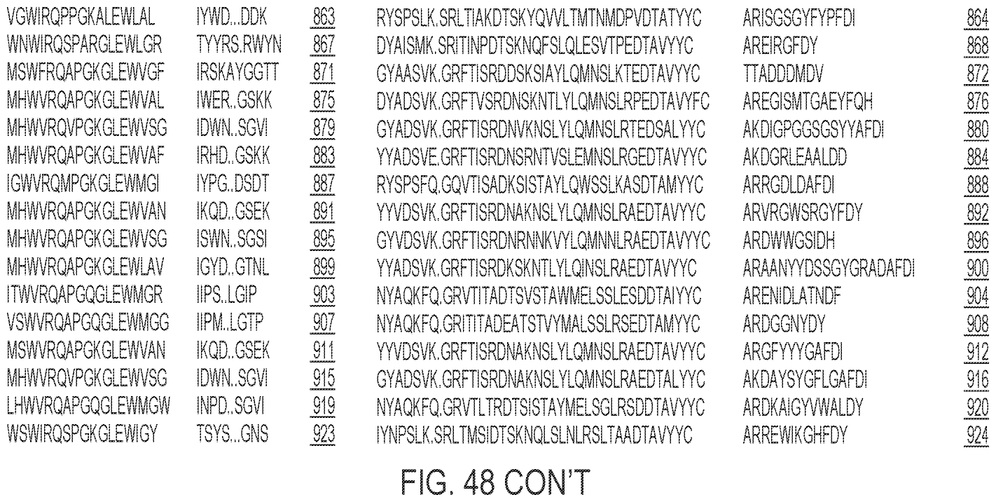

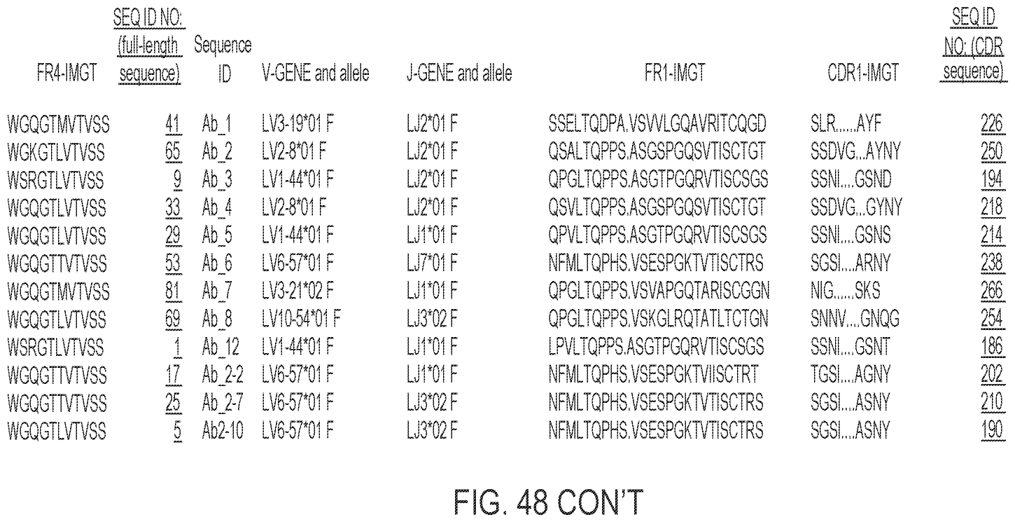

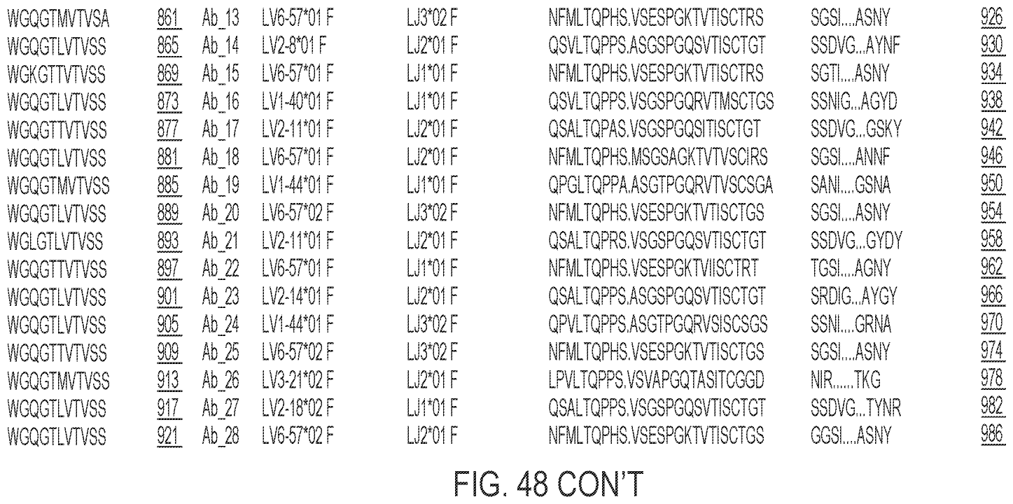

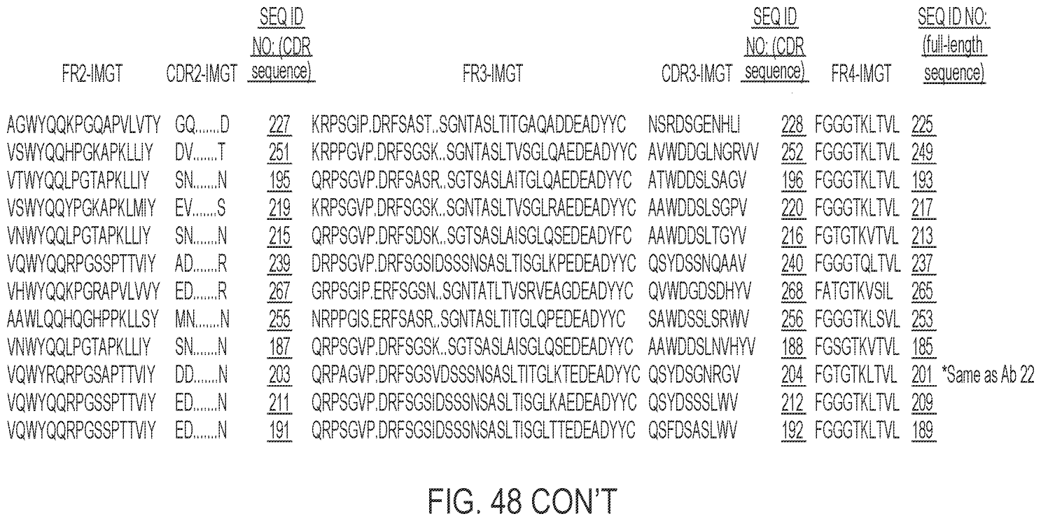

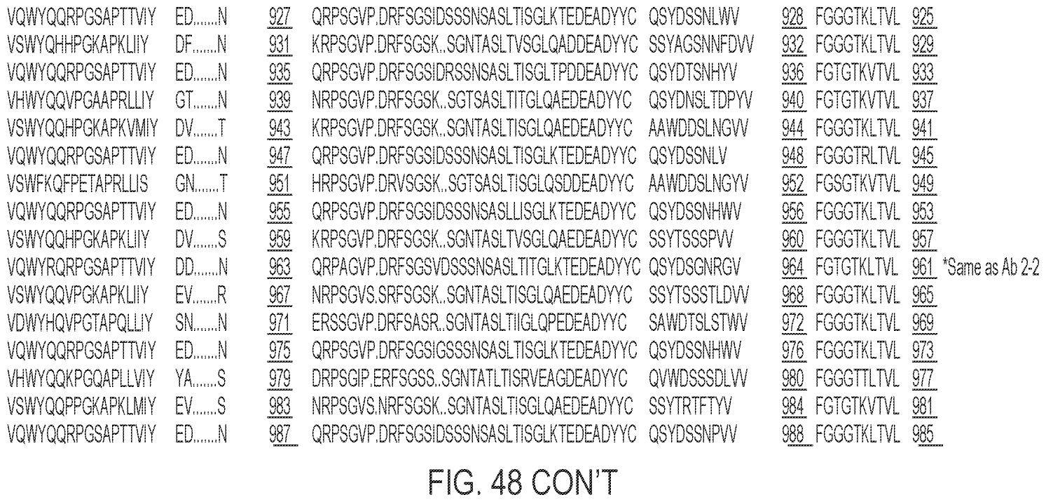

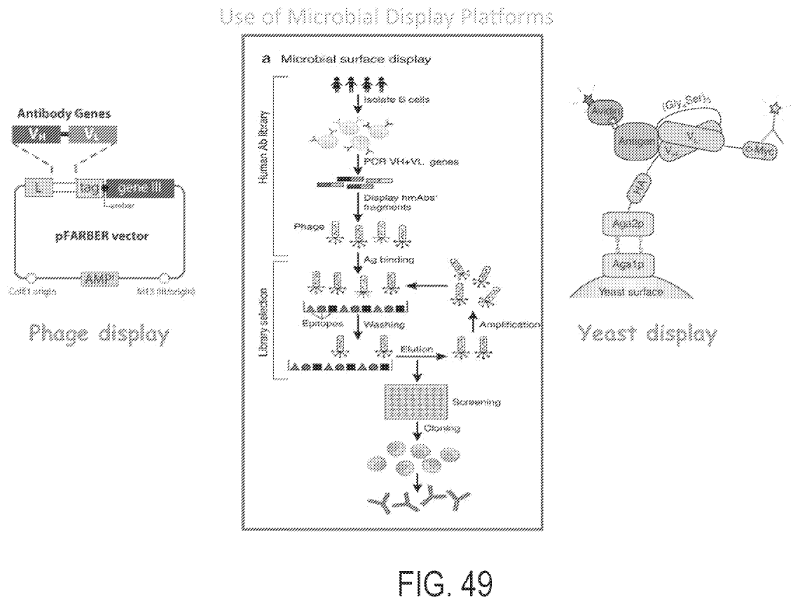

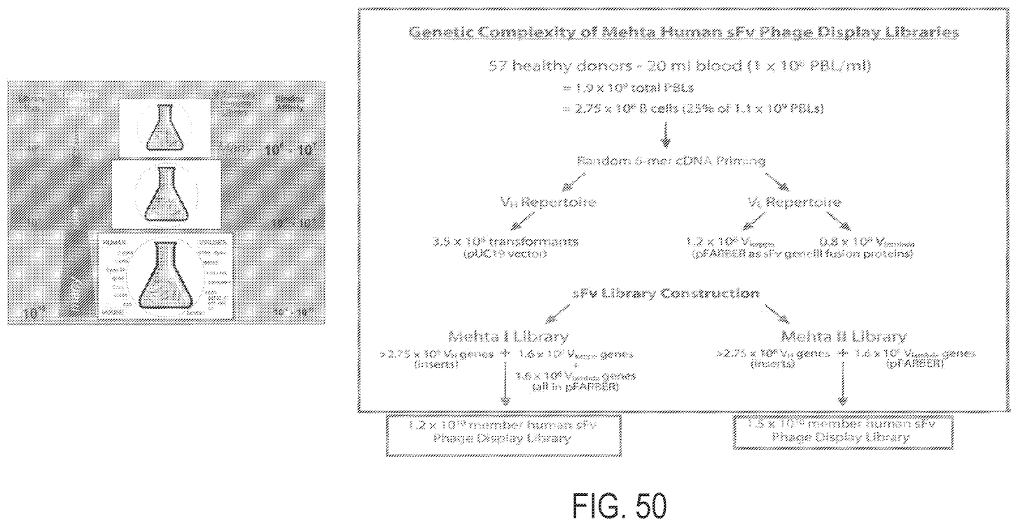

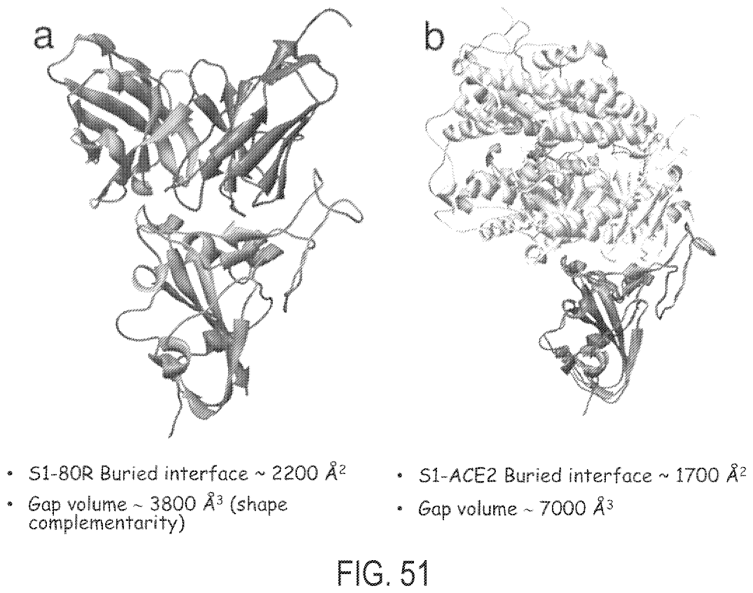

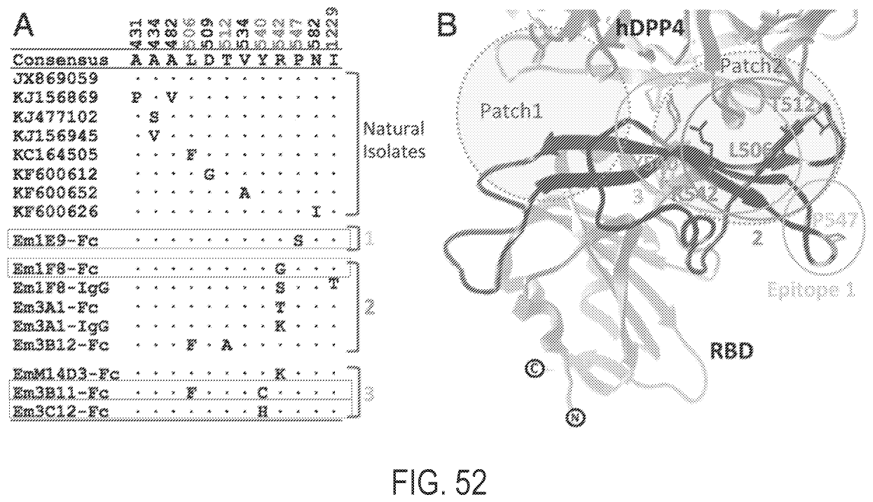

Referring to FIG. 10, for example, plate coated with RBD-Fc (0.5 μg/ml, 100 μl)

Step 1: phage added at 5E11 particles/ml, except RBD-E1-B3 was at 1E12 to move to shoulder of binding curve

Step 2: ACE2-his was added in 2×serial dilutions starting at 2 μg/ml

Step 3: phage binding was detected by anti M13-HRP; (ACE2 curve is detected via anti-his-hrp, no phage added)

S1-RBD-T1-B12 was used as a negative control as it was not expected to block RBD-ACE2 binding. As expected, the anti-M13 signal is flat here, showing that ACE2 did not have any effect on phage binding.

E1-B3 and T1-F7 show distinct decreases in phage binding at higher concentrations of ACE2 indicating that there is competition for the binding site. T1-F4 shows a small decrease in signal at higher concentrations but is not as clear.

### Example 3—Kinetic Analysis of Selected scFv-Fc Candidates

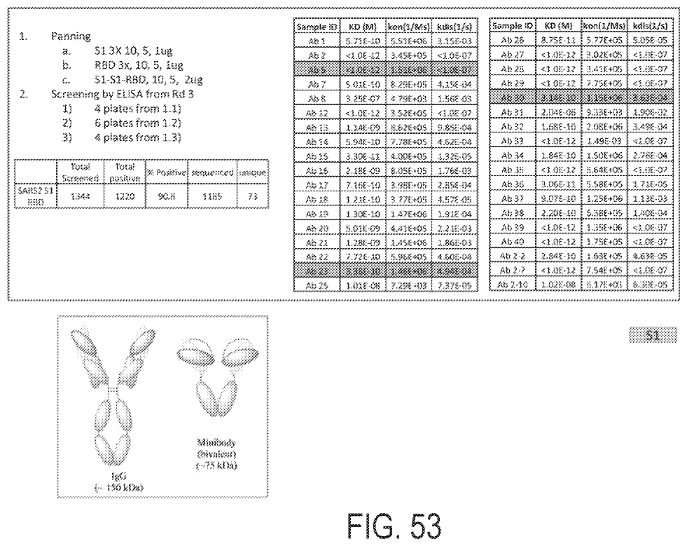

FIG. 53 shows result from SARS-2 S1/RBD panning. Three rounds of panning for anti-SARS-2 S1/RBD antibodies was done using recombinantly expressed soluble protein. a large number of antibodies with varying kinetic properties. The concentration of the coating protein was decreased with each round to increase the affinity of the antibodies. Two campaigns were straight panning with all three rounds against the same target protein (with different purification tags). The third campaign started with two rounds against S1, followed by a 3rd panning against the RBD protein to enrich for antibodies against the RBD. Screening was performed by picking 1344 colonies and culturing them in 2×YT media. The phage supernatants were then tested via ELISA against RBD-Fc protein (the S1 panning was also screened against S1). From our screens, >90% of the selected colonies were positive for binding to either S1 or RBD. Sequencing of all positive samples yielded 73 unique clones. Kinetic analysis was performed via BLI. Octet sensors were coated with low density of biotinylated S1 protein to minimize scFv-Fc cross walking. Antibodies with low levels of binding here were found to bind biotinylated RBD coated sensors significantly better. Without wishing to be bound by theory, this could be due to the size difference of S1 versus RBD, the RBD coated sensors have a larger number of RBD molecules available for binding. Additionally, the large size of the S1 protein forces the binding even further from the sensor surface which also contributes to lower signal.

Pseudovirus neutralization was performed with SARS-2 spike pseudovirus and 293T-ACE2 transduced cells. Kinetic data for both of these abs reveal tight binding antibodies with minimal disassociation. Additionally, epitope mapping reveals that they have similar but slightly different competition patterns (FIG. 54). Ab 12 successfully competes with Abs 14, 15, 19, 26, and 27. While Ab 27 also competes with Abs 12, 14, and 15, it does not compete with Ab 19 or Ab 26. Without wishing to be bound by theory, the antibodies bind similar epitopes but have a different angle of approach.

### Example 4—Neutralization Studies

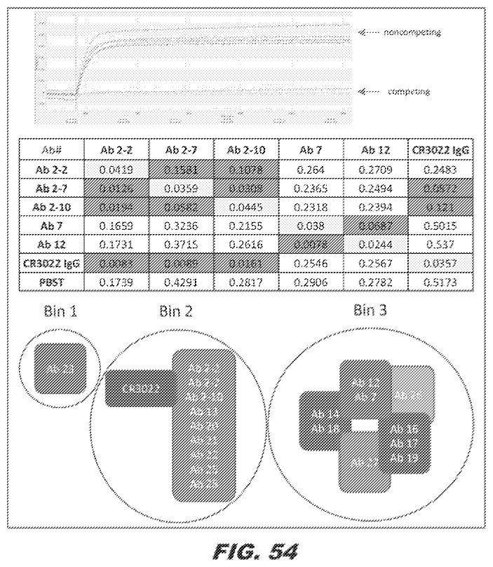

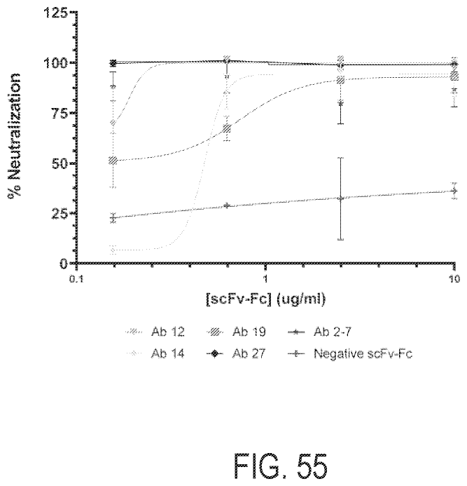

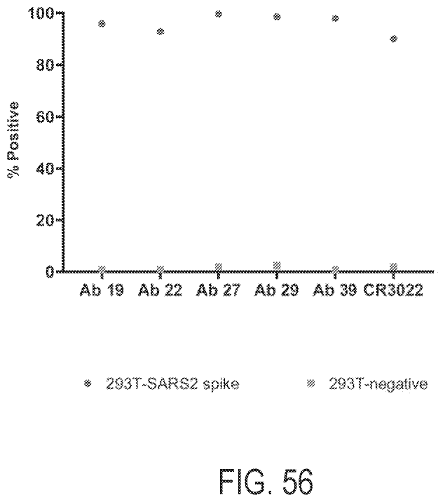

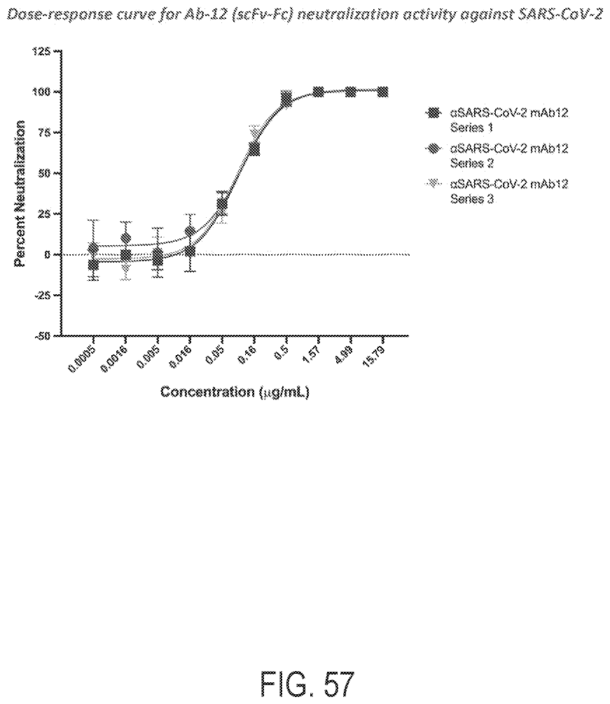

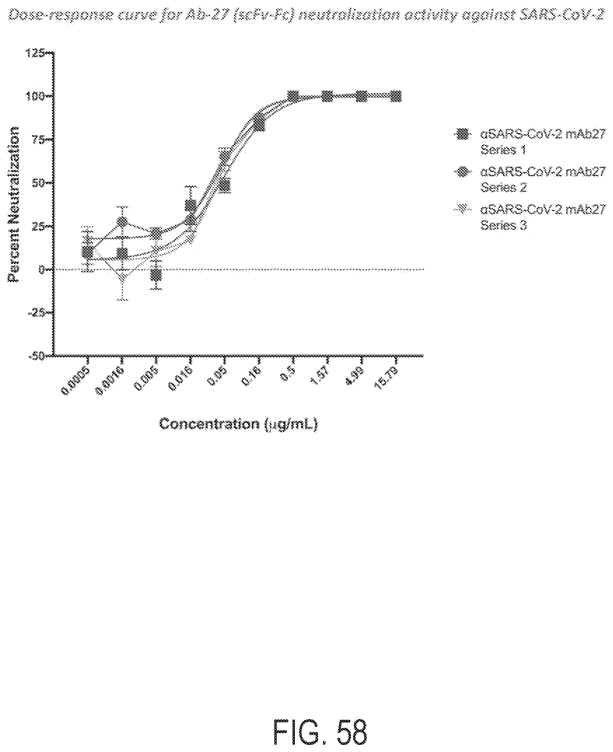

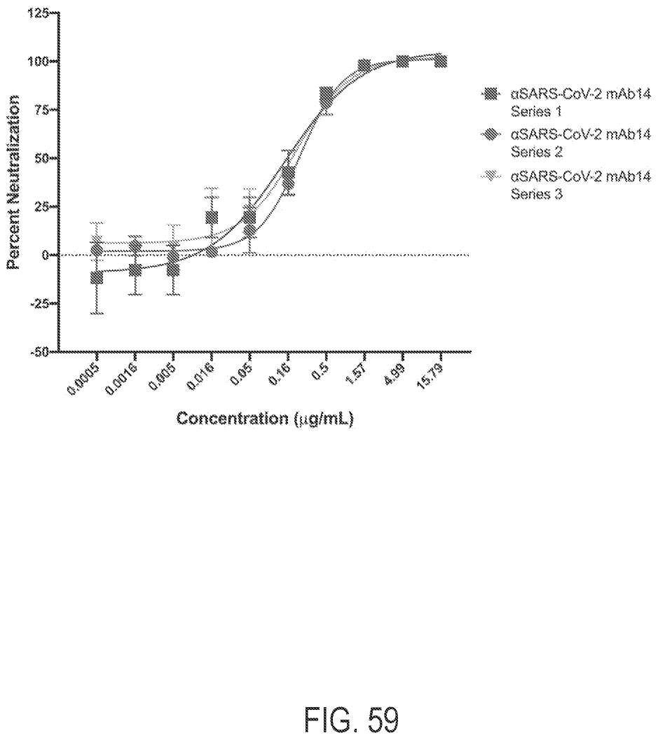

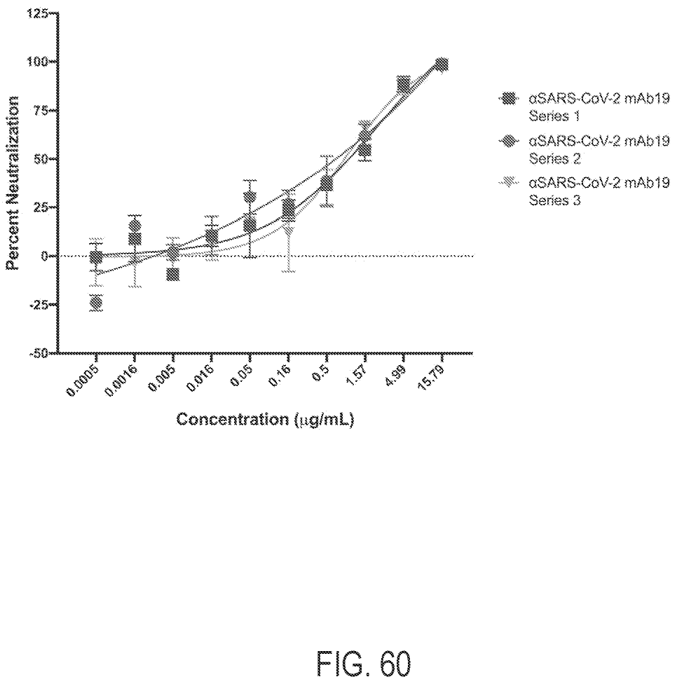

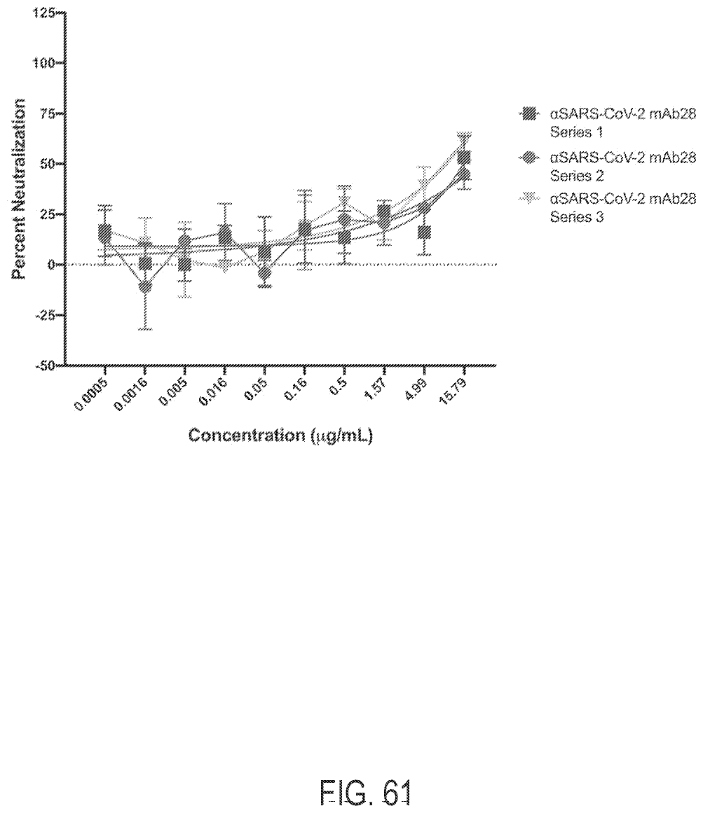

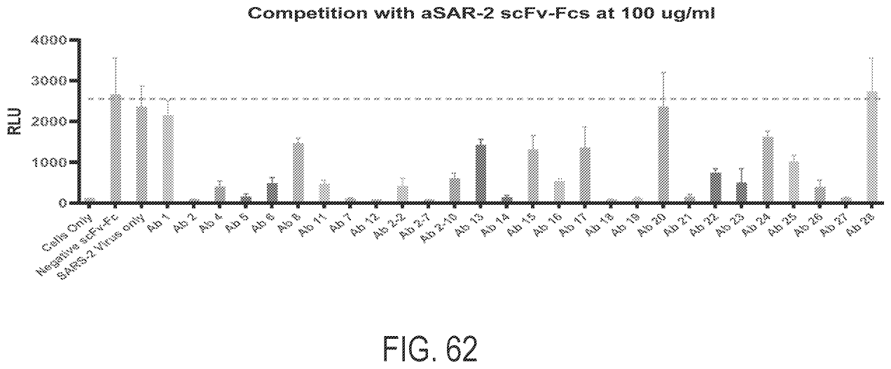

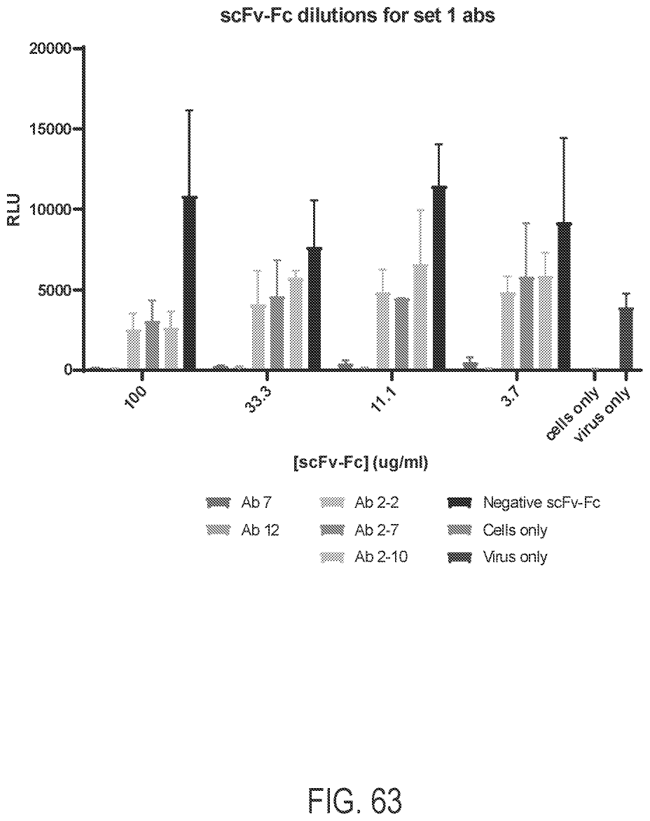

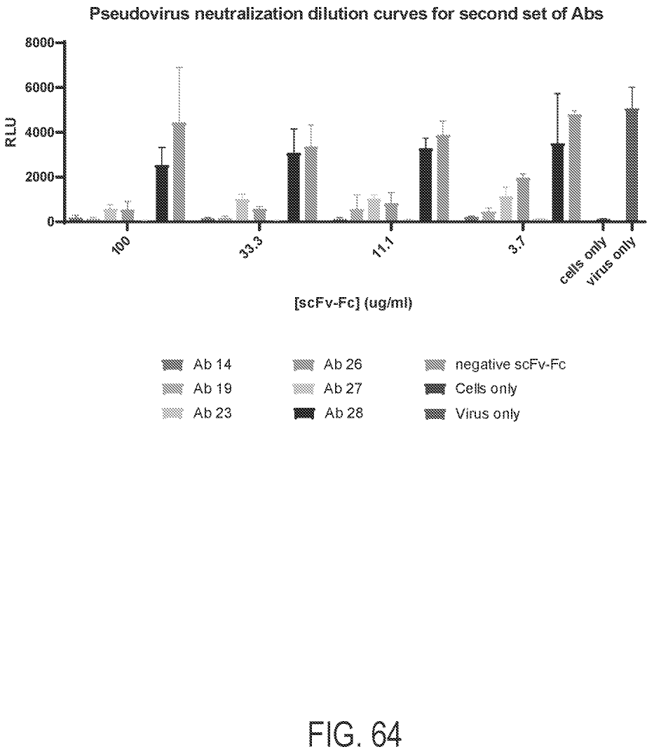

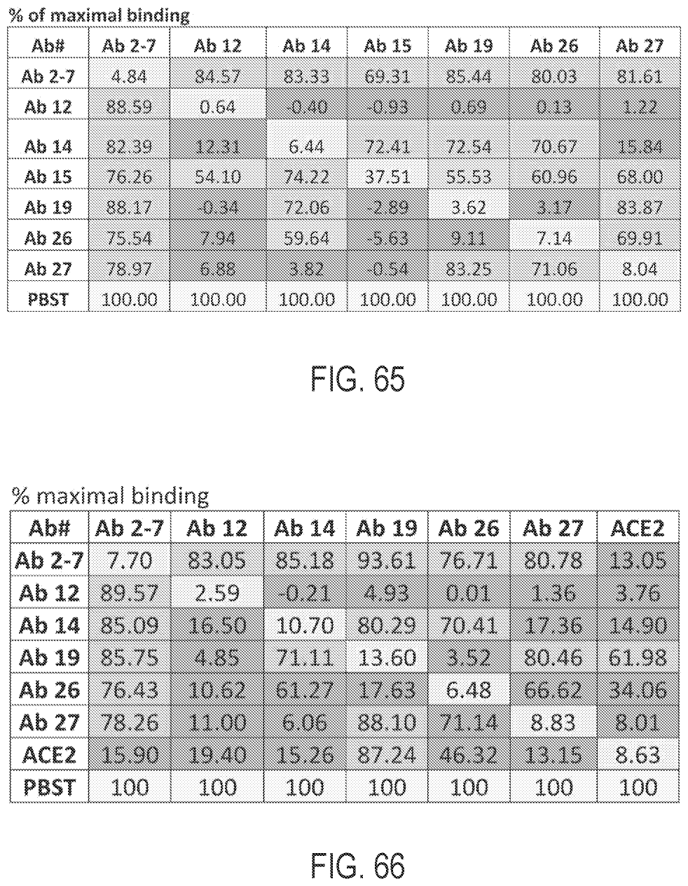

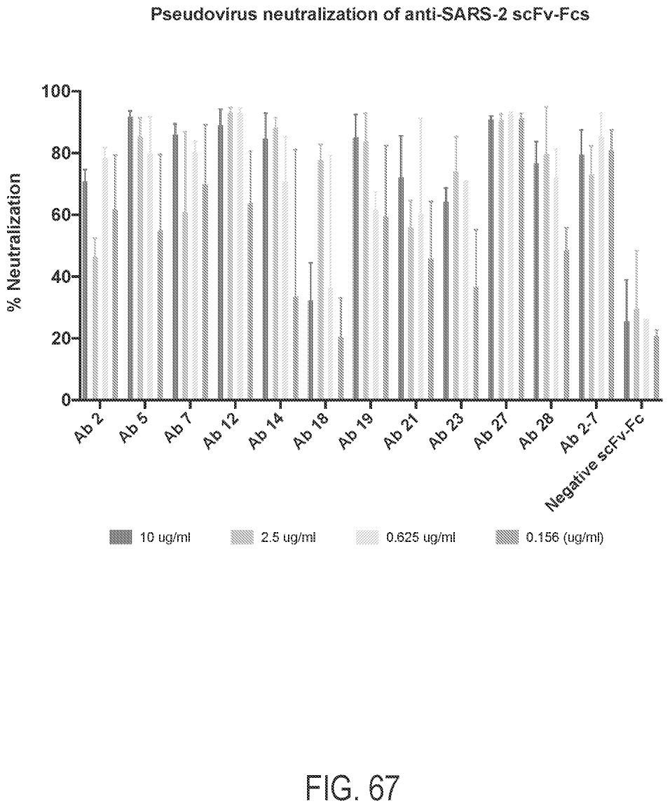

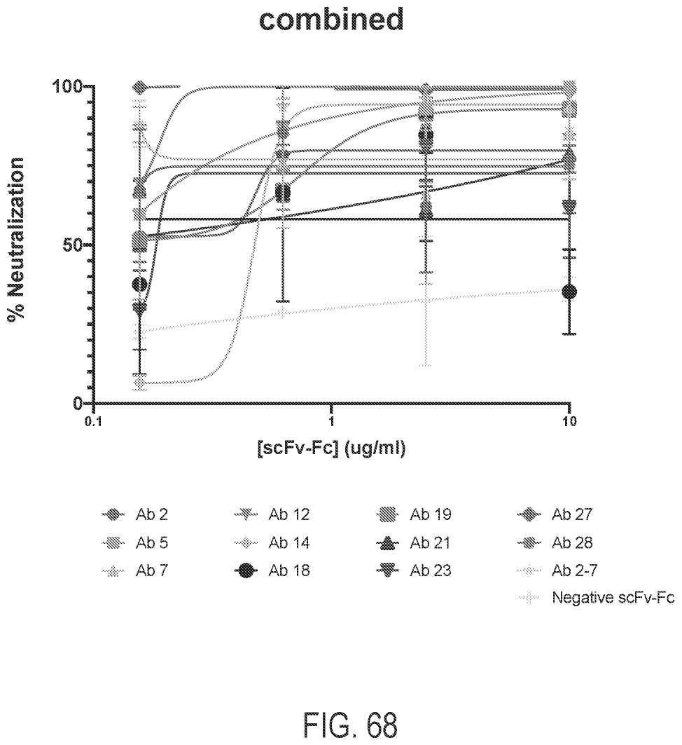

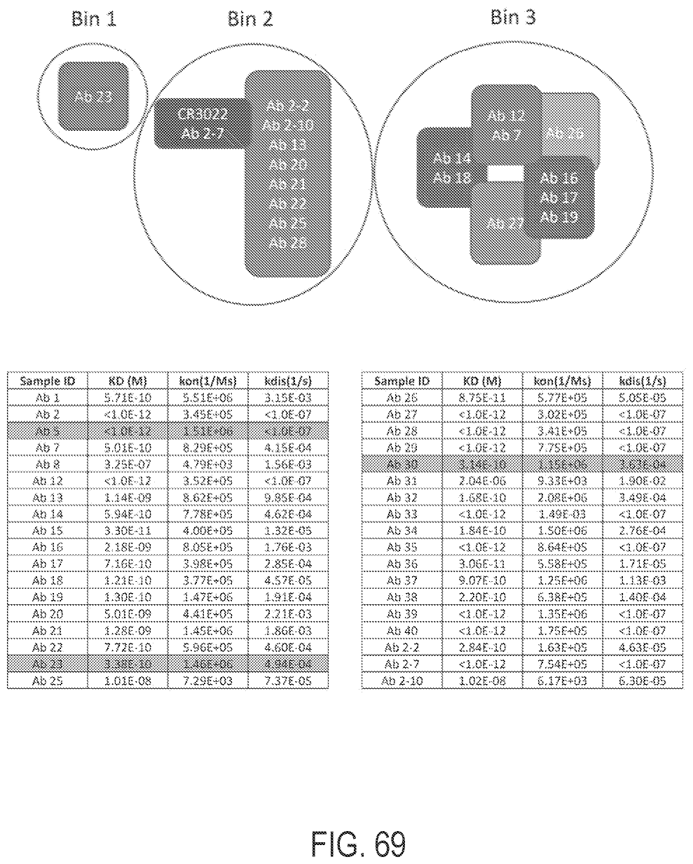

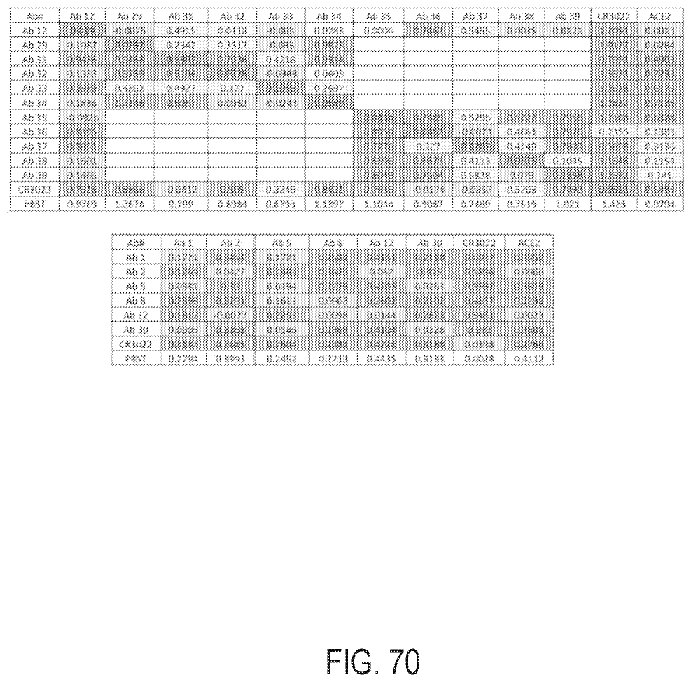

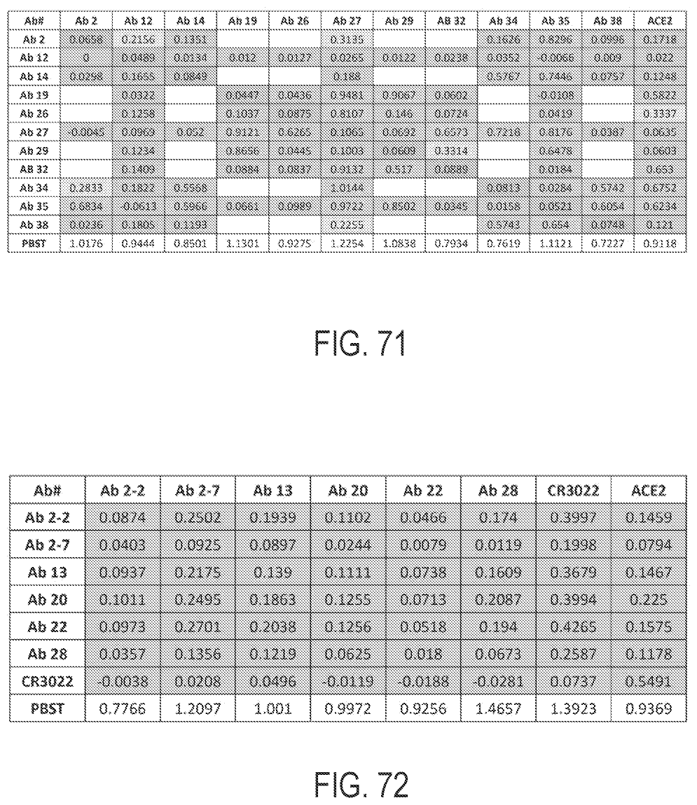

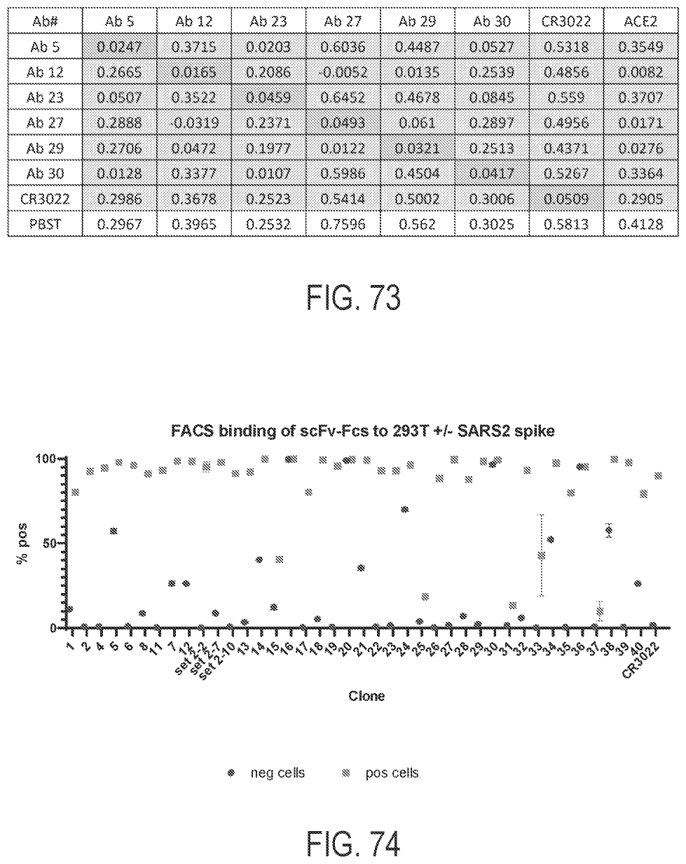

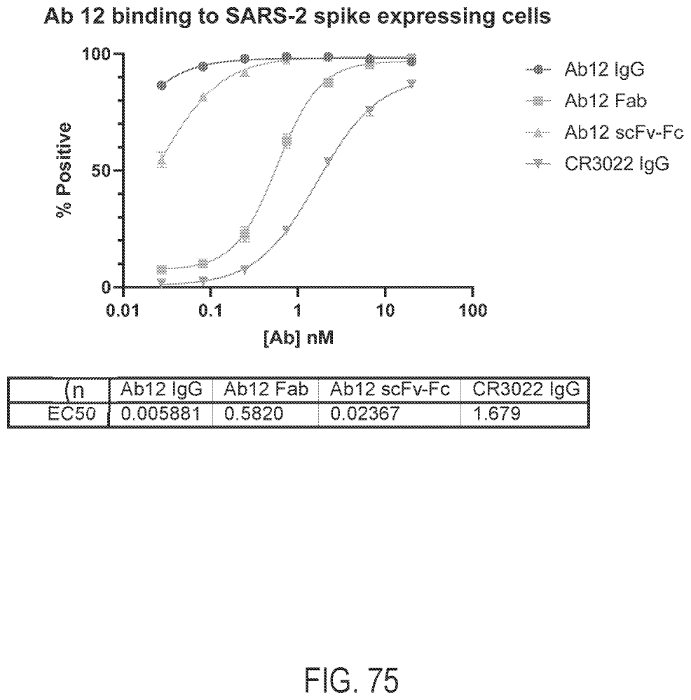

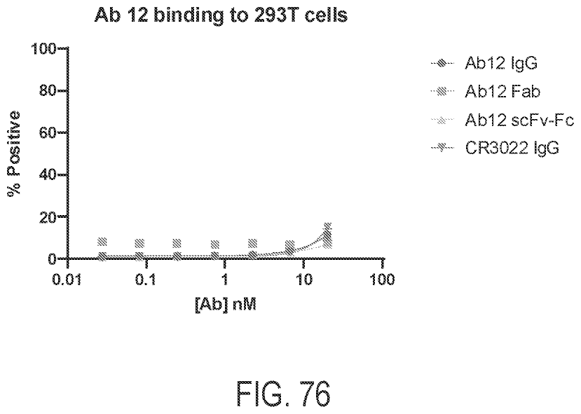

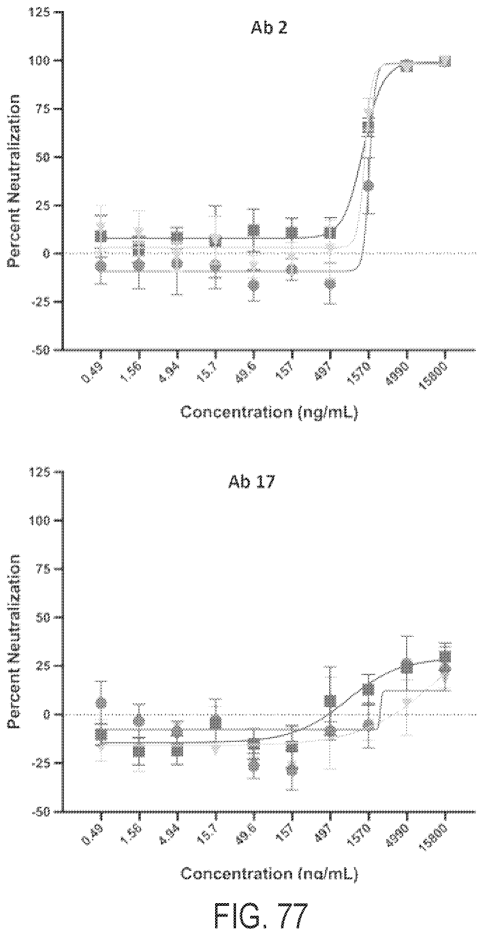

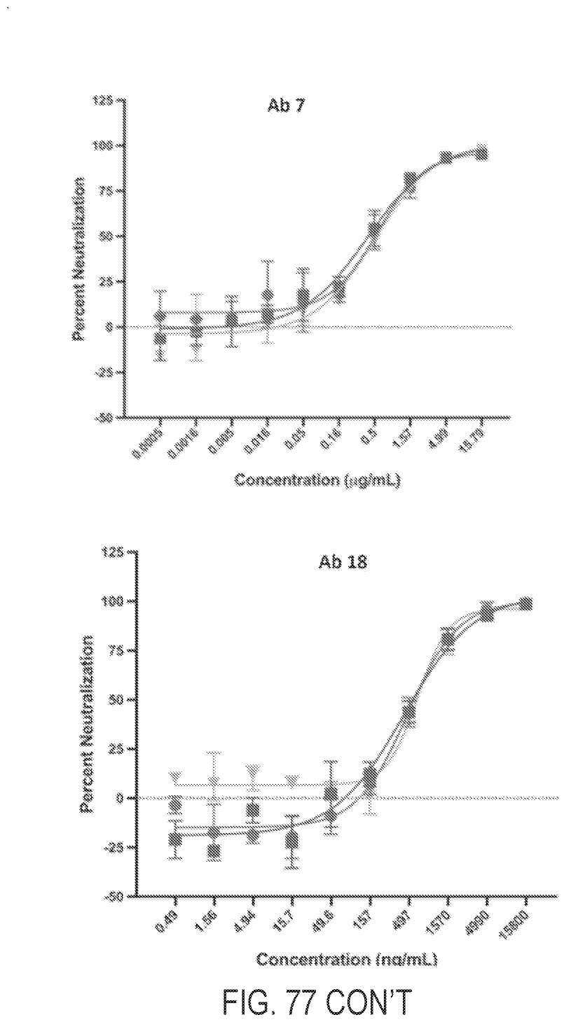

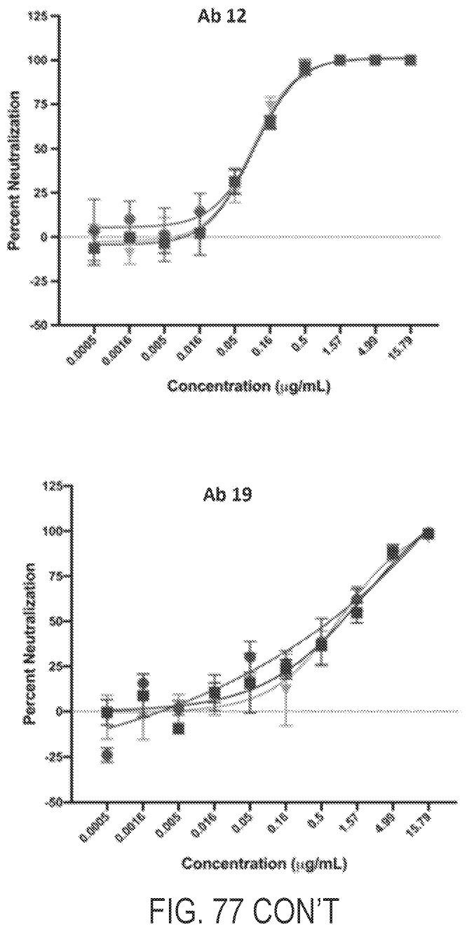

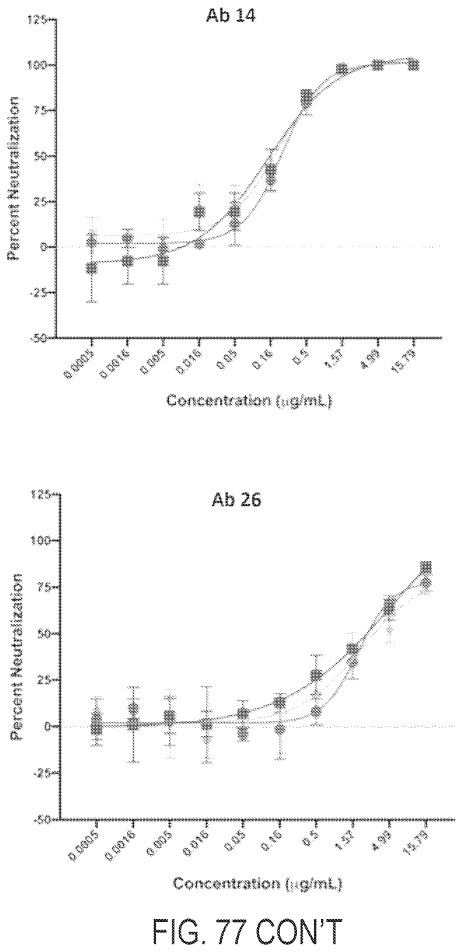

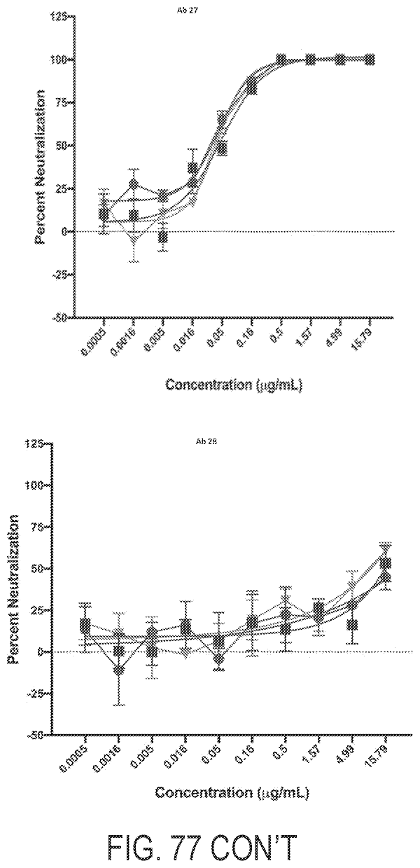

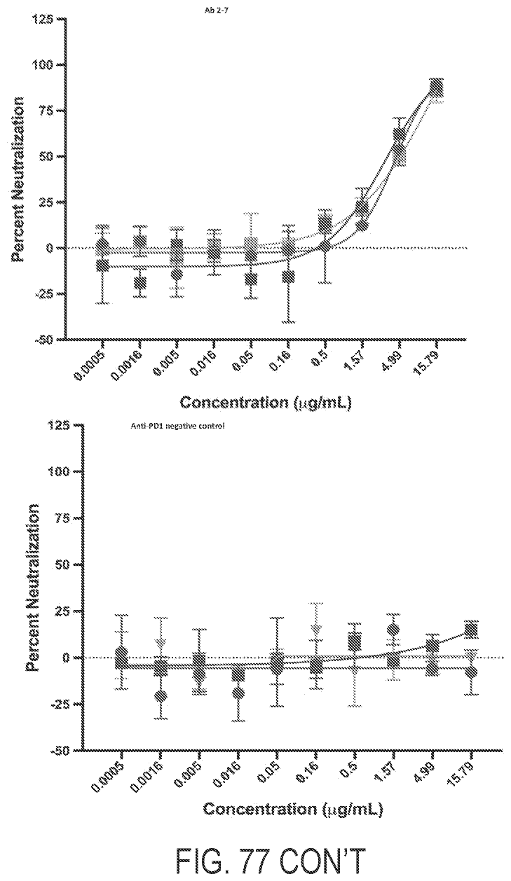

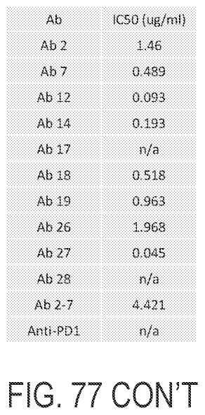

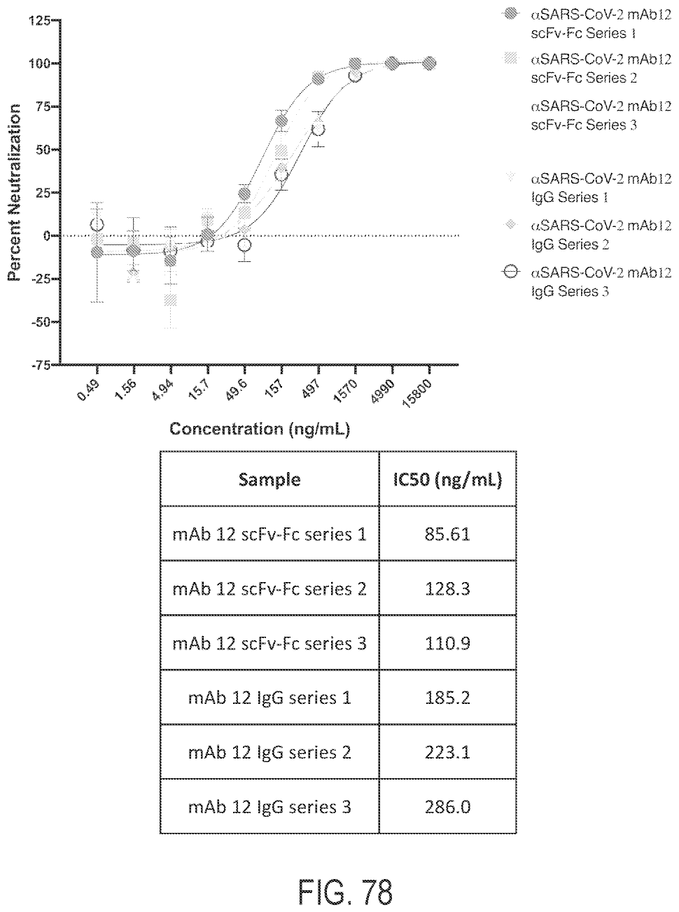

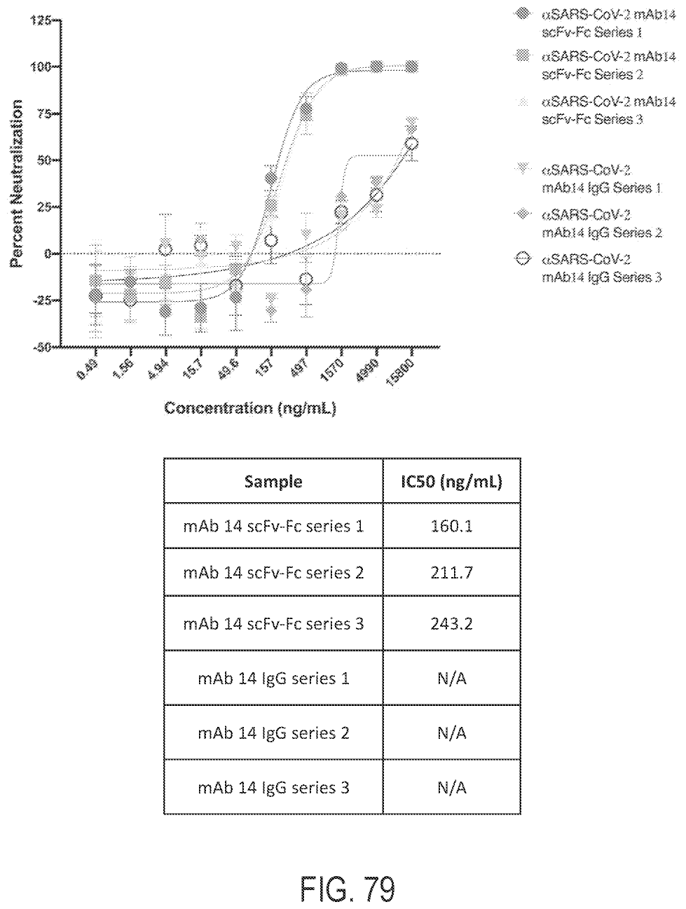

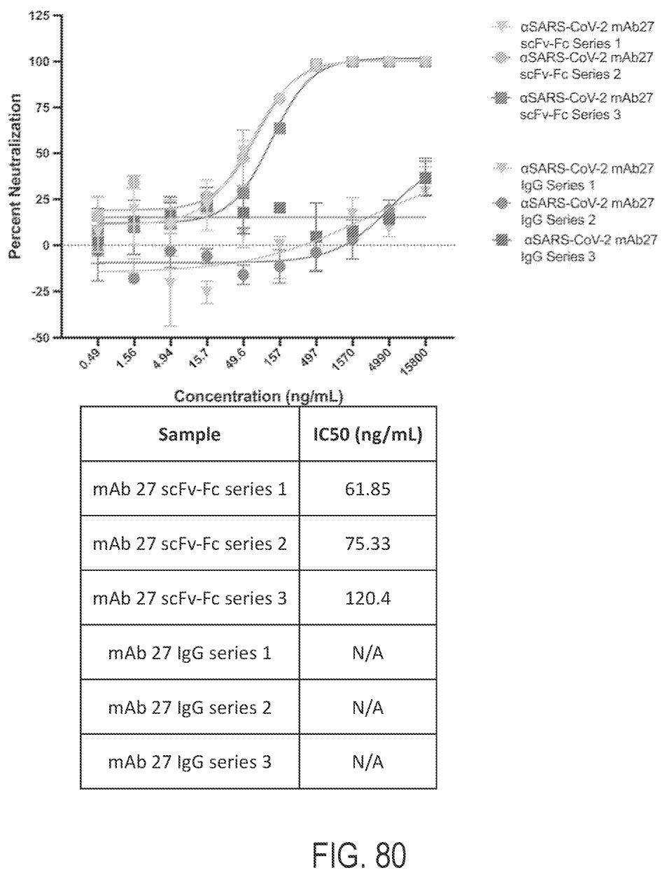

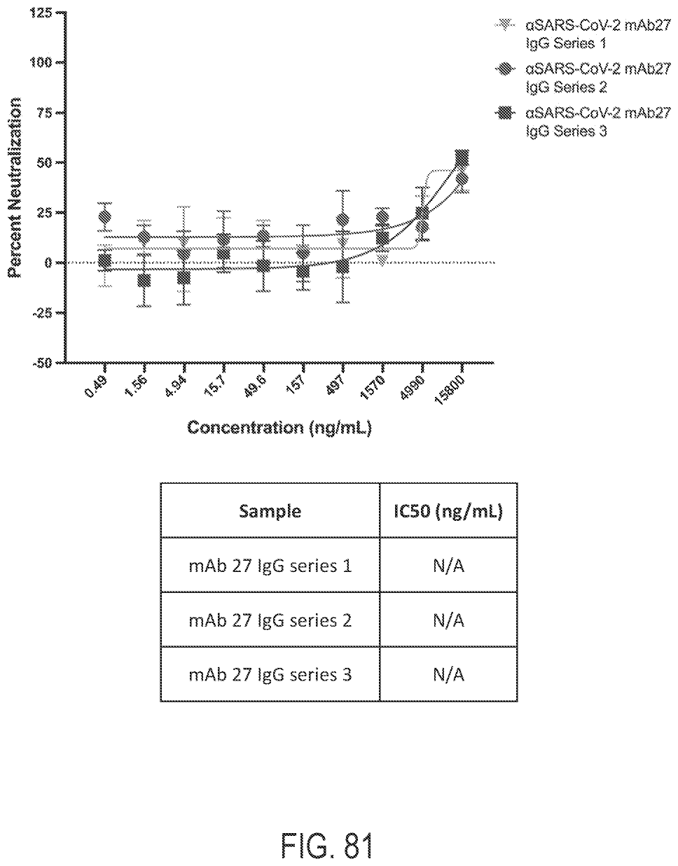

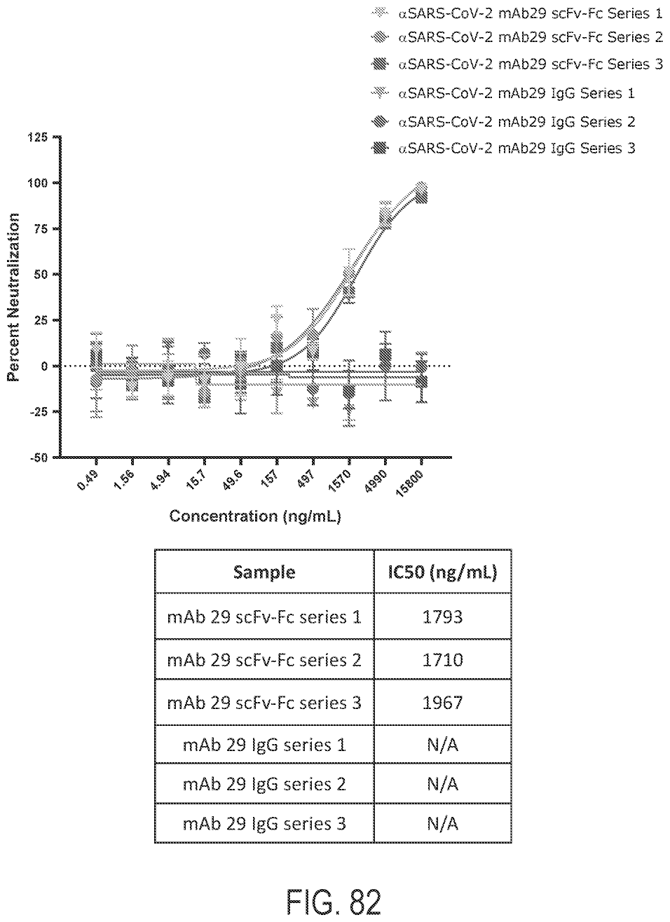

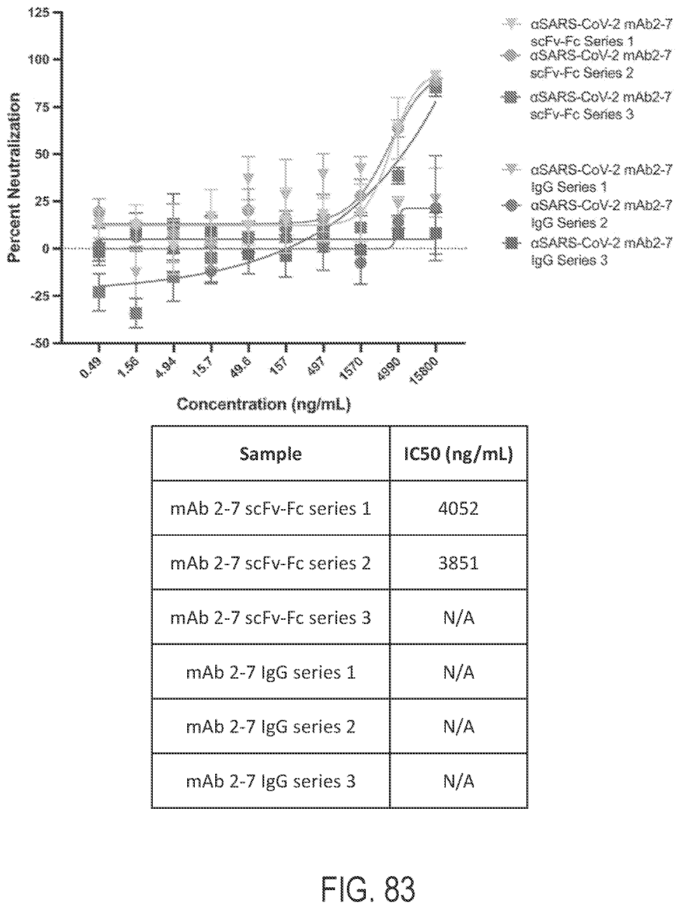

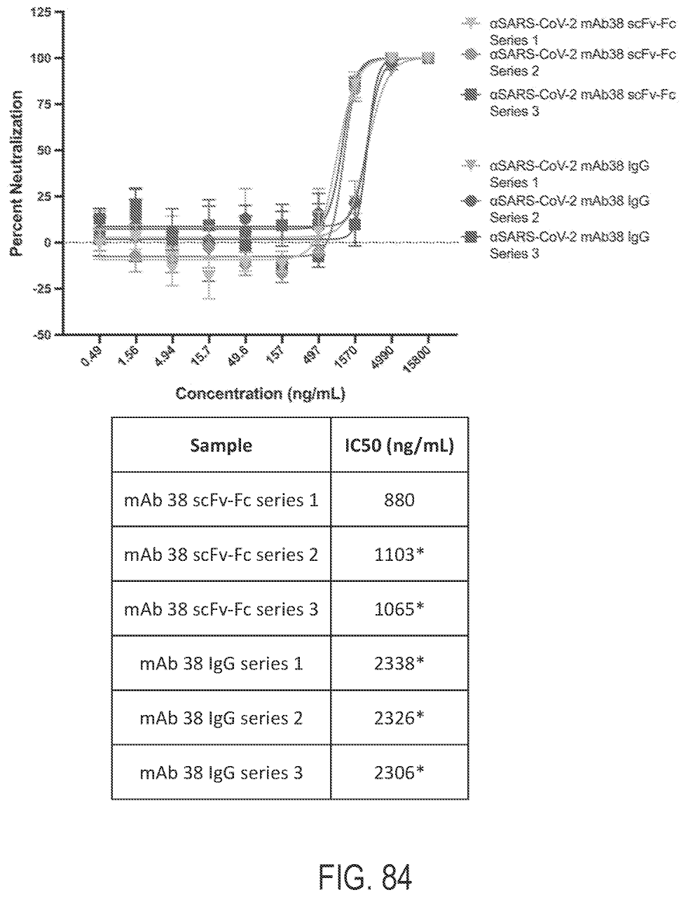

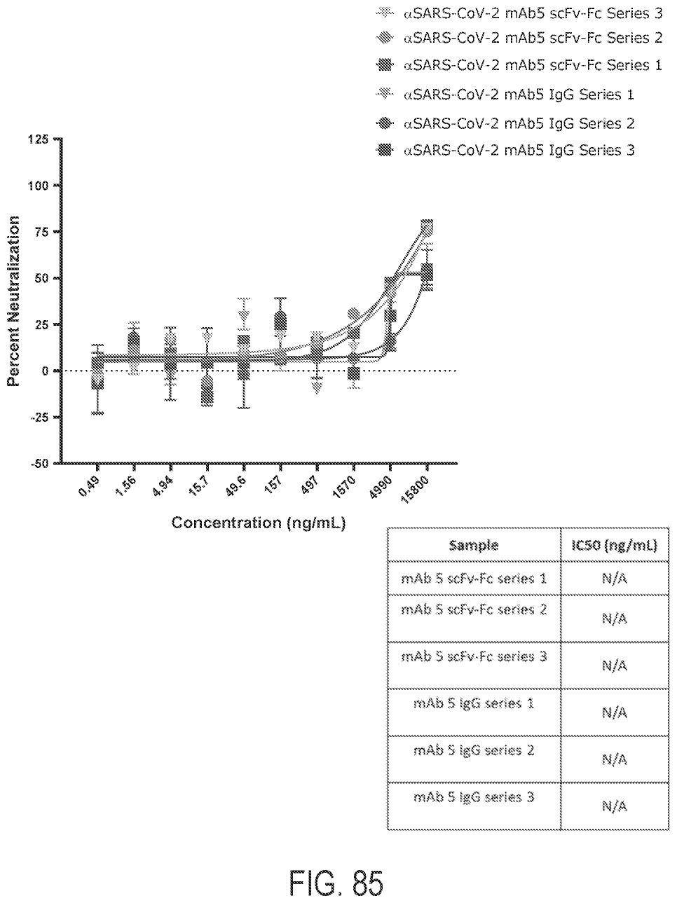

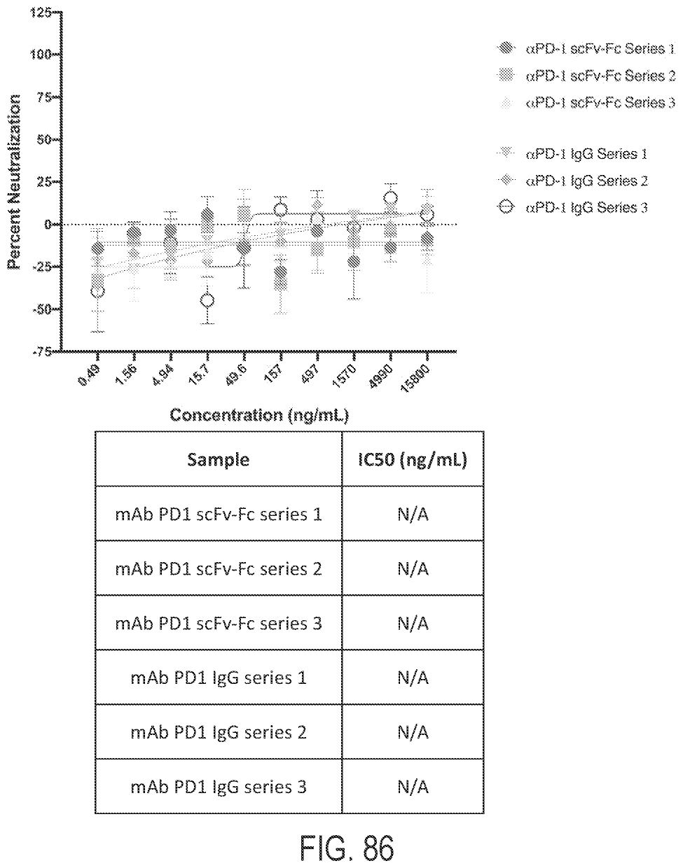

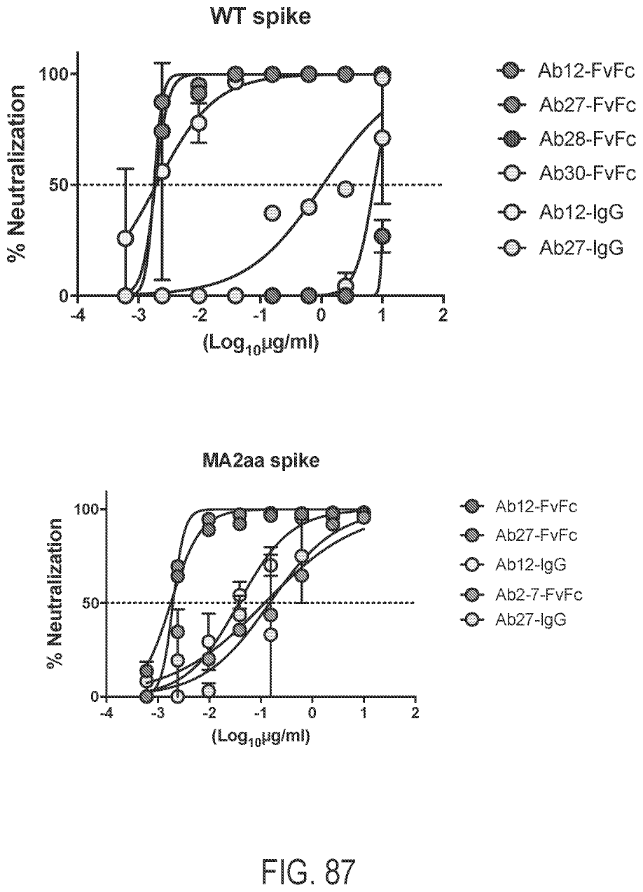

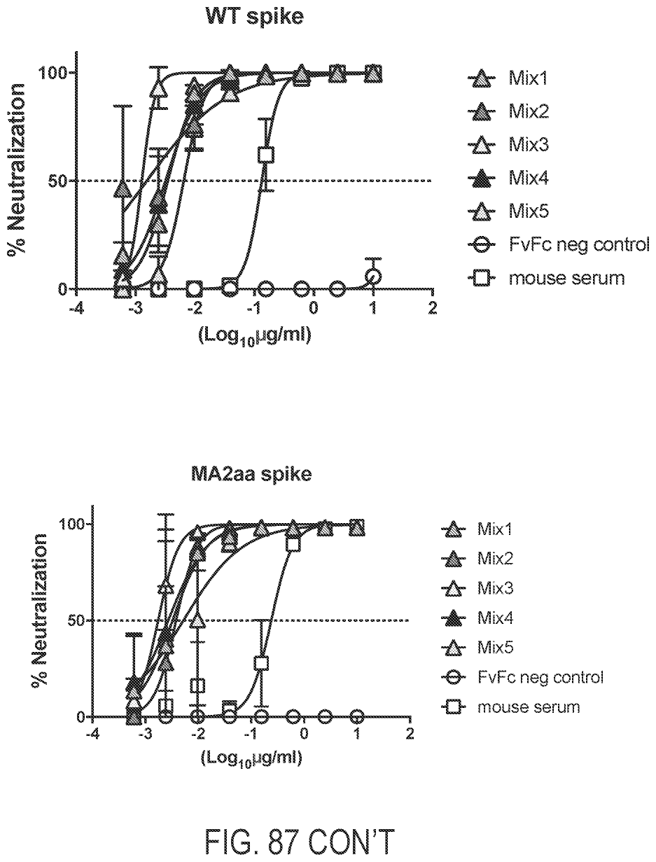

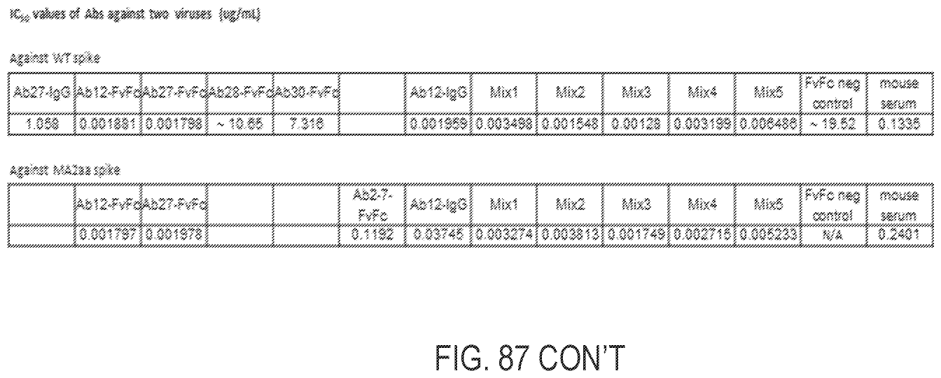

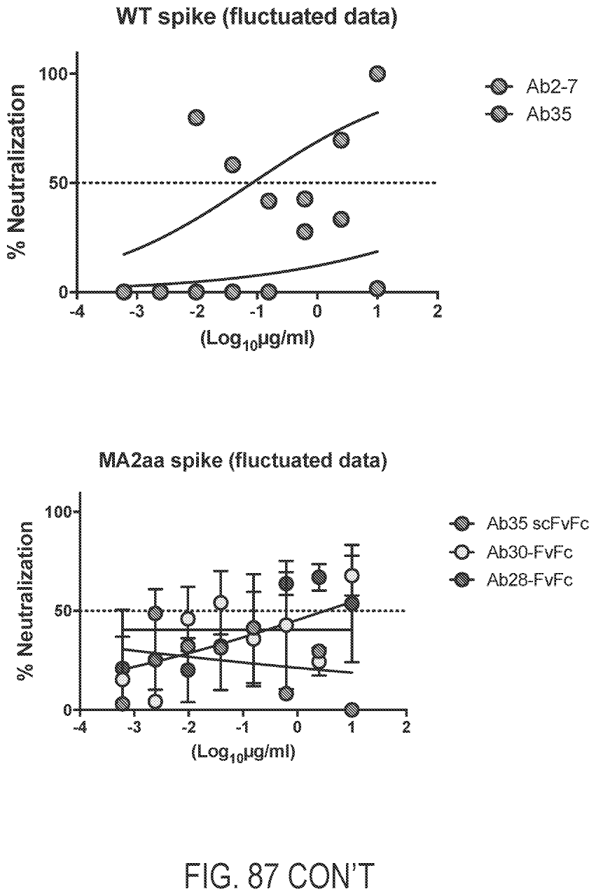

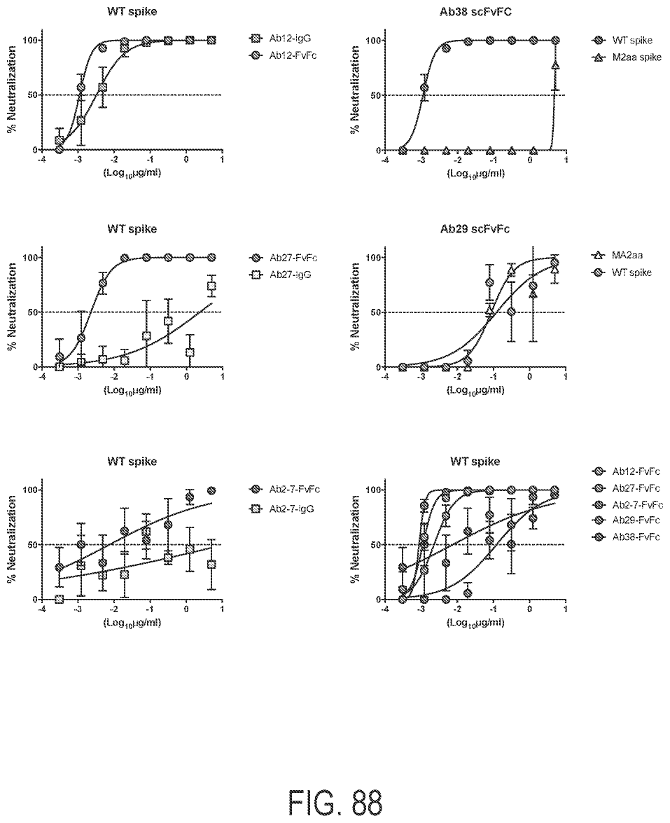

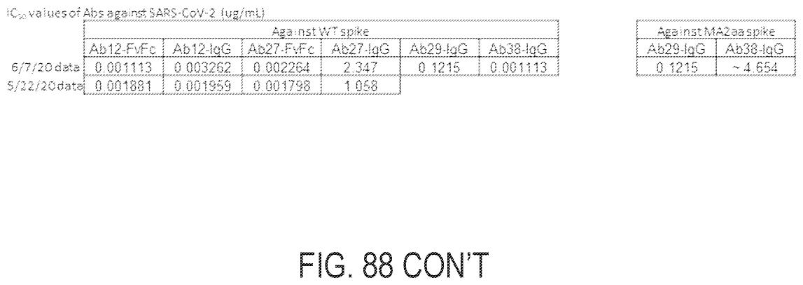

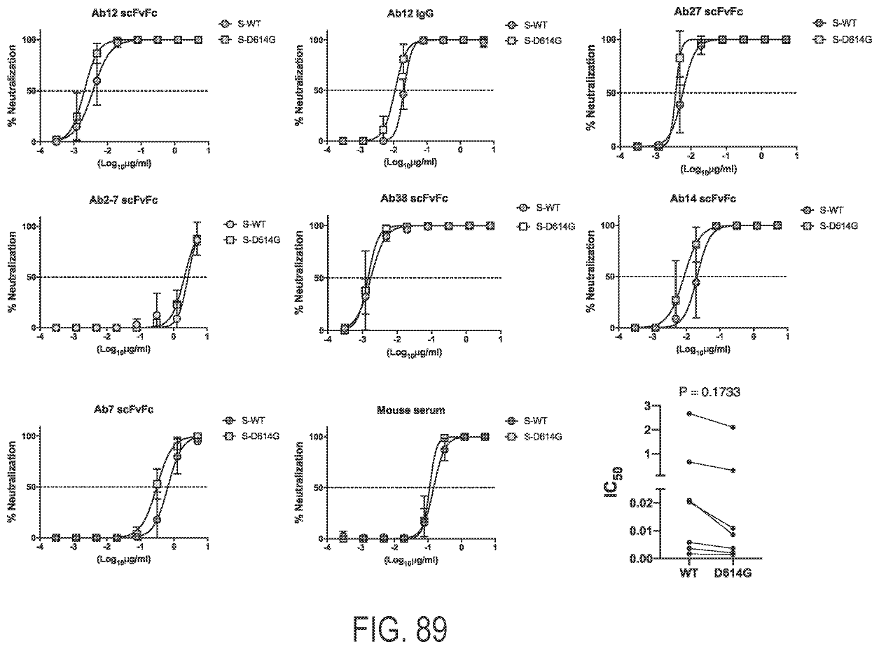

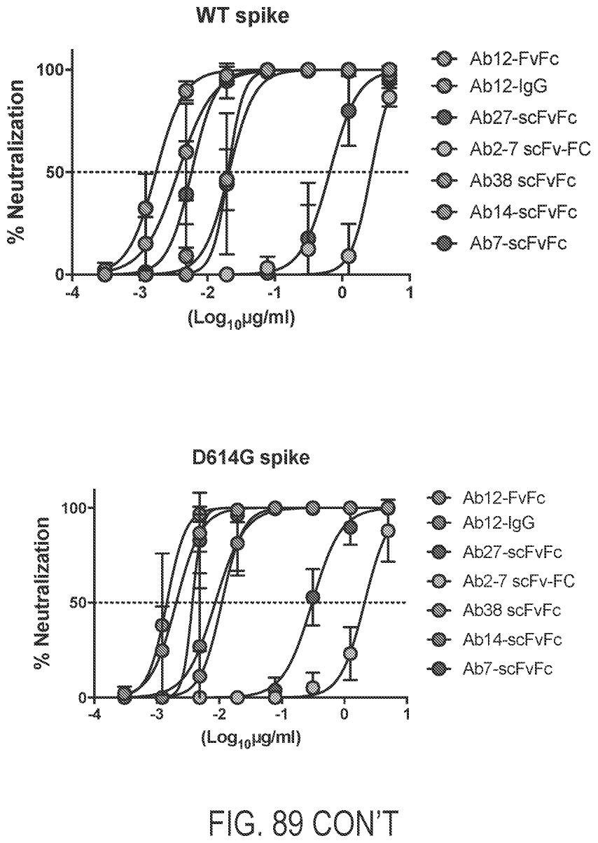

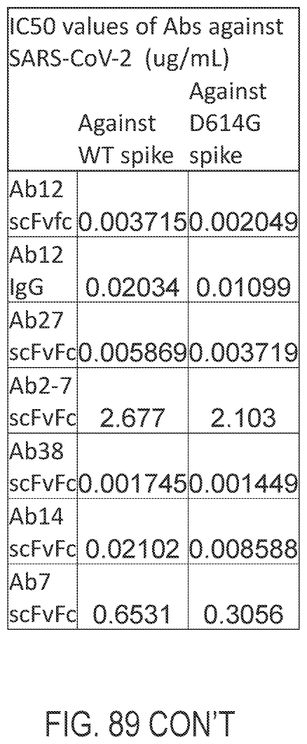

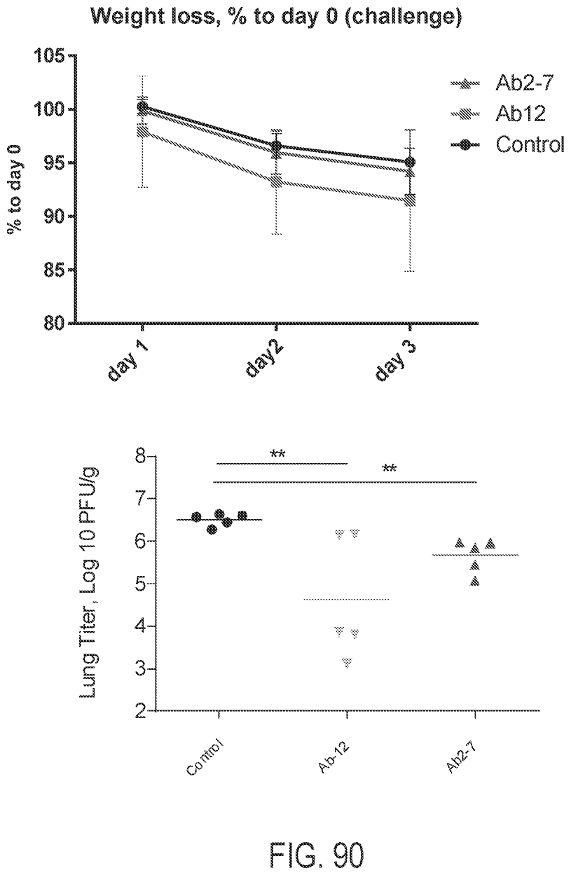

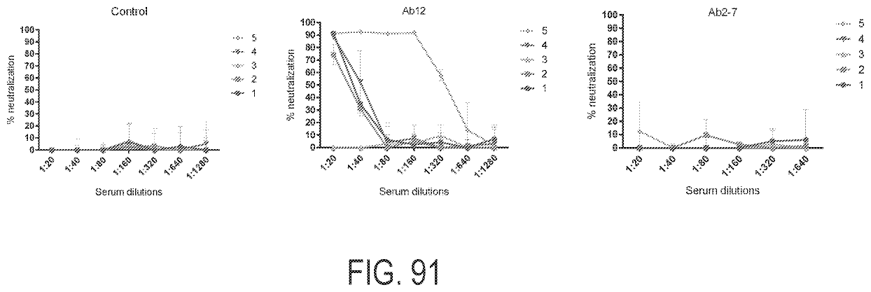

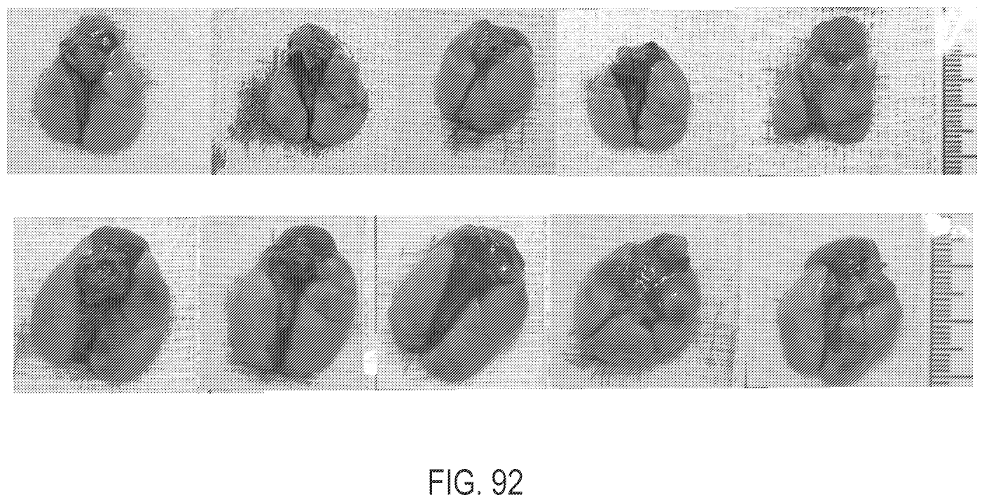

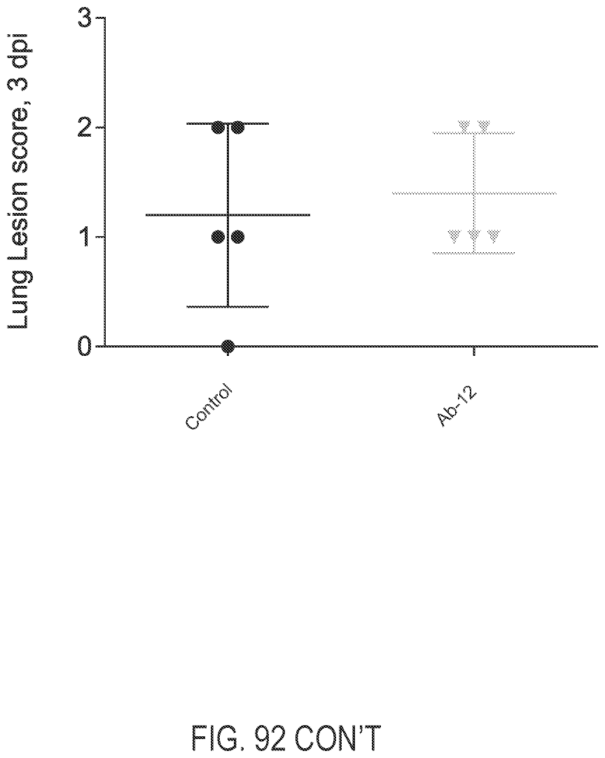

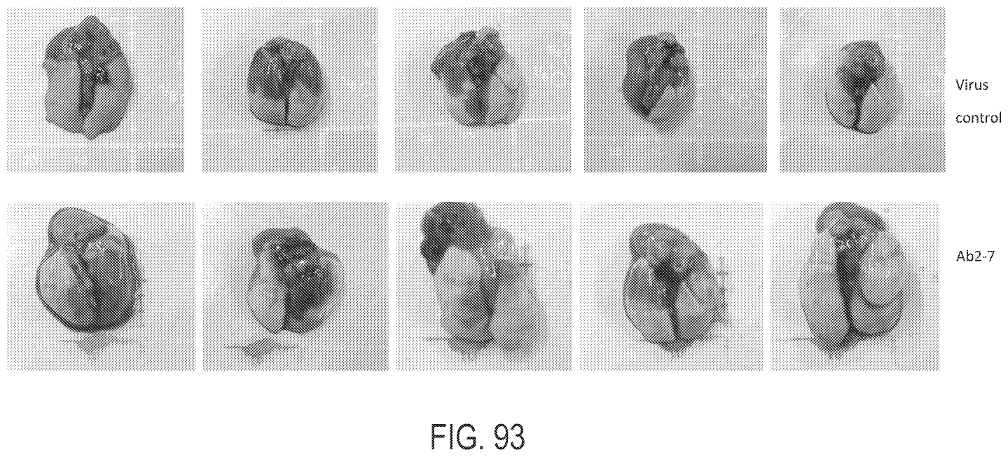

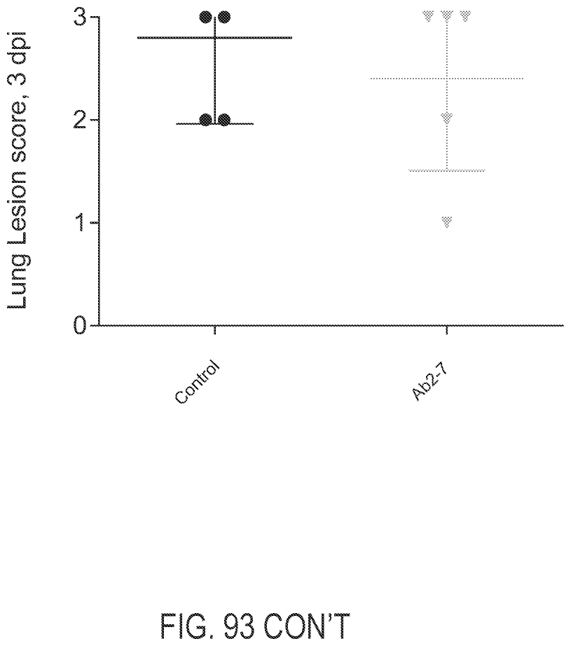

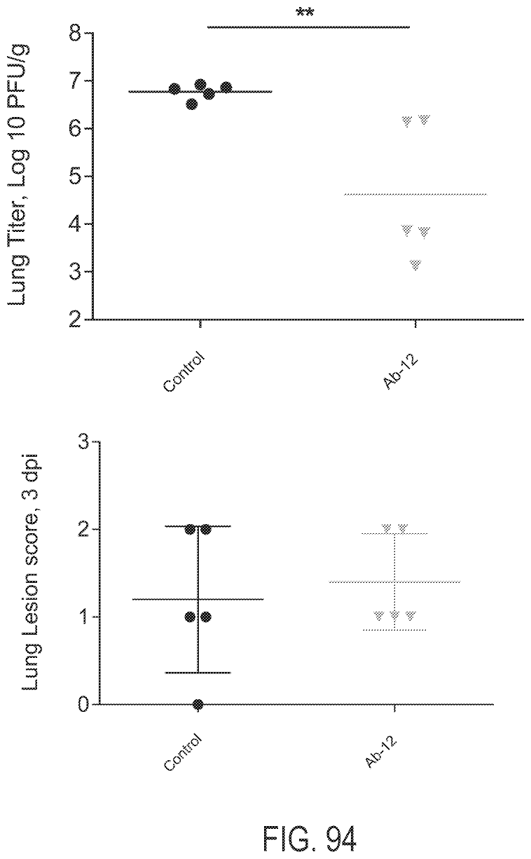

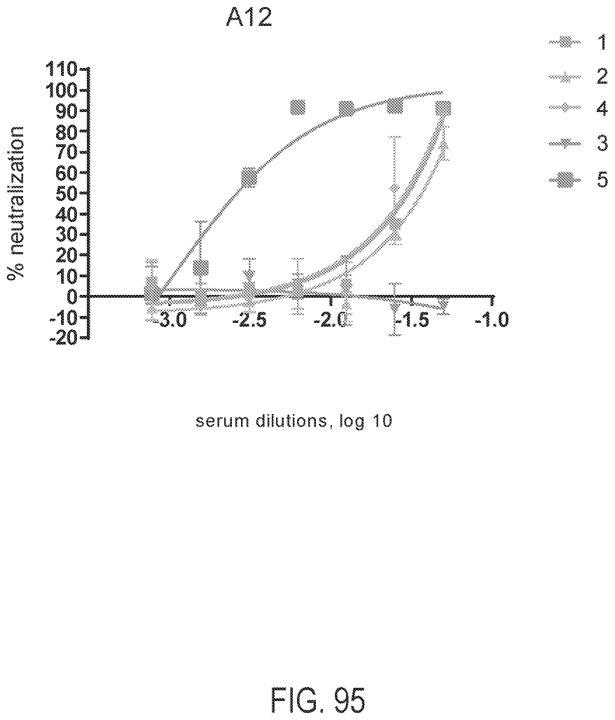

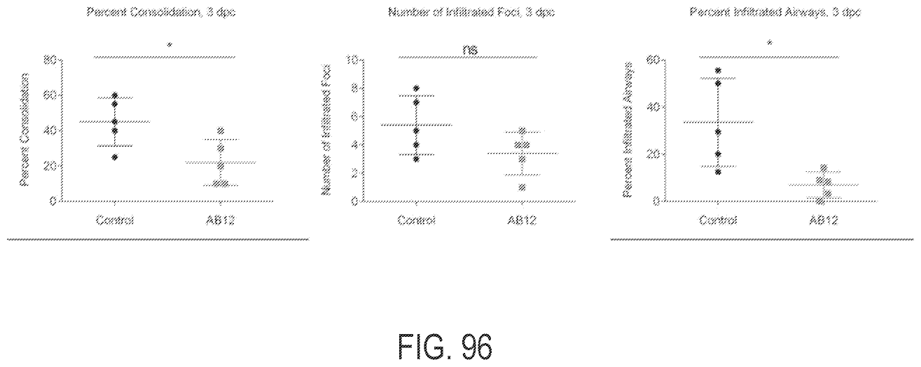

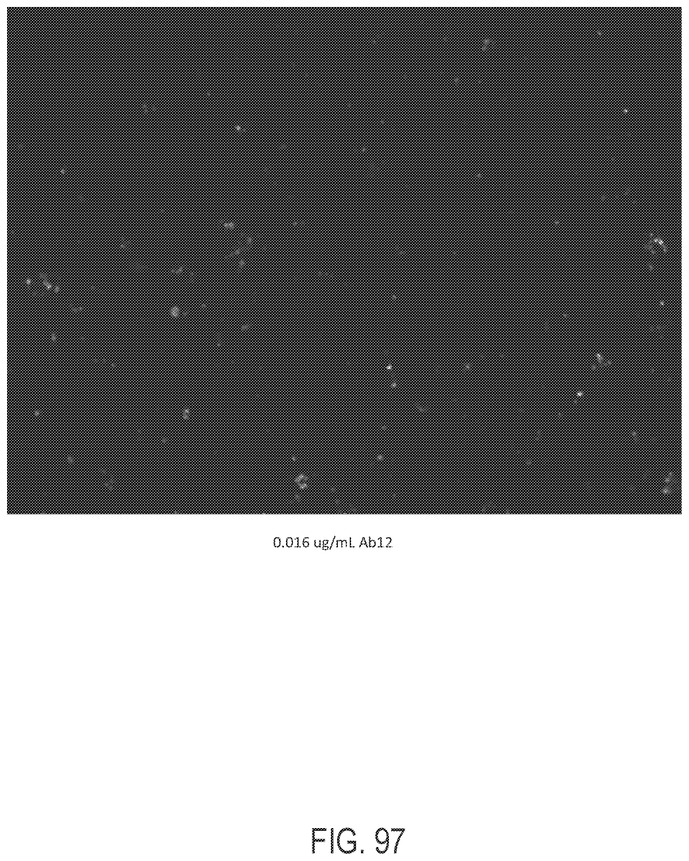

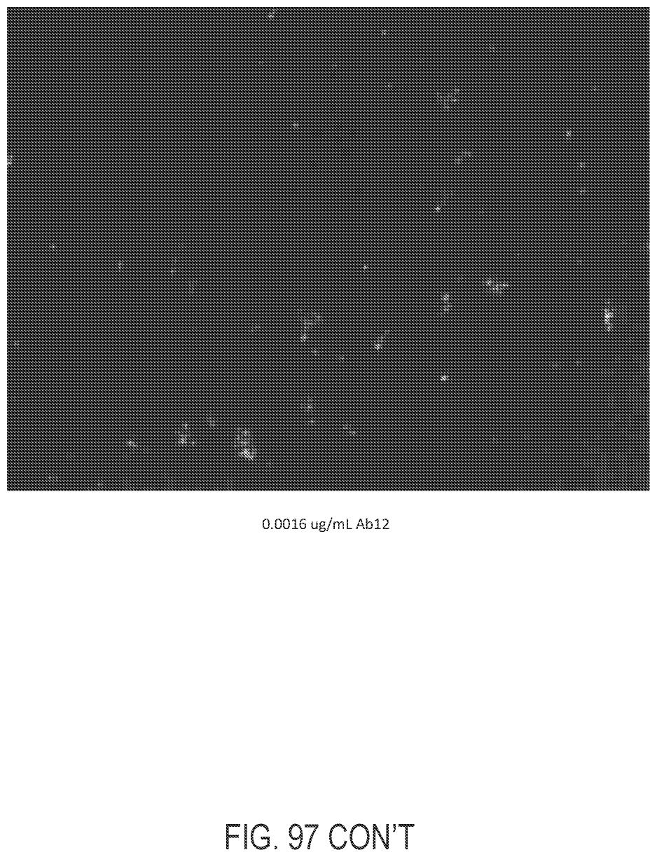

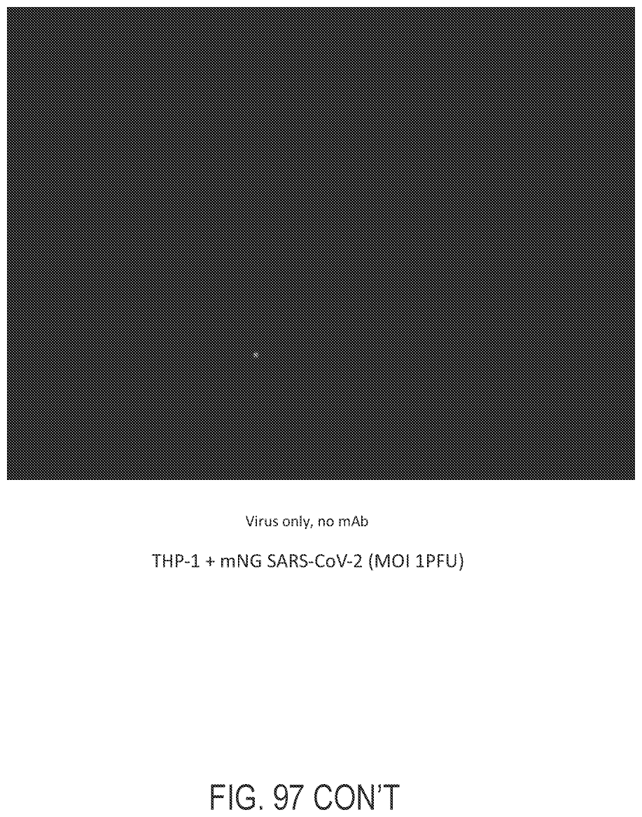

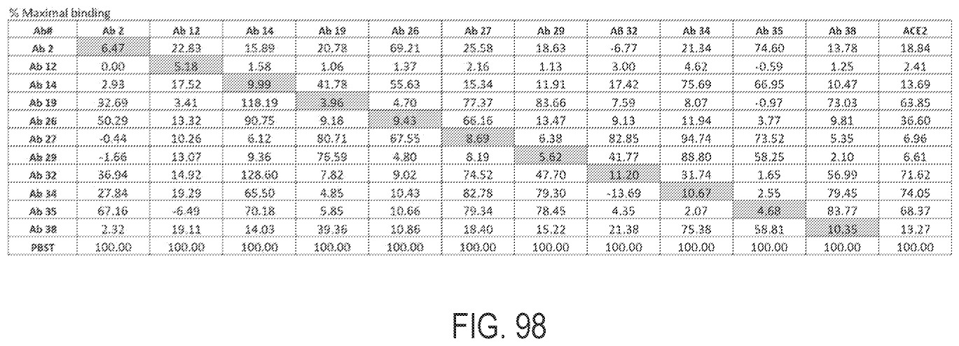

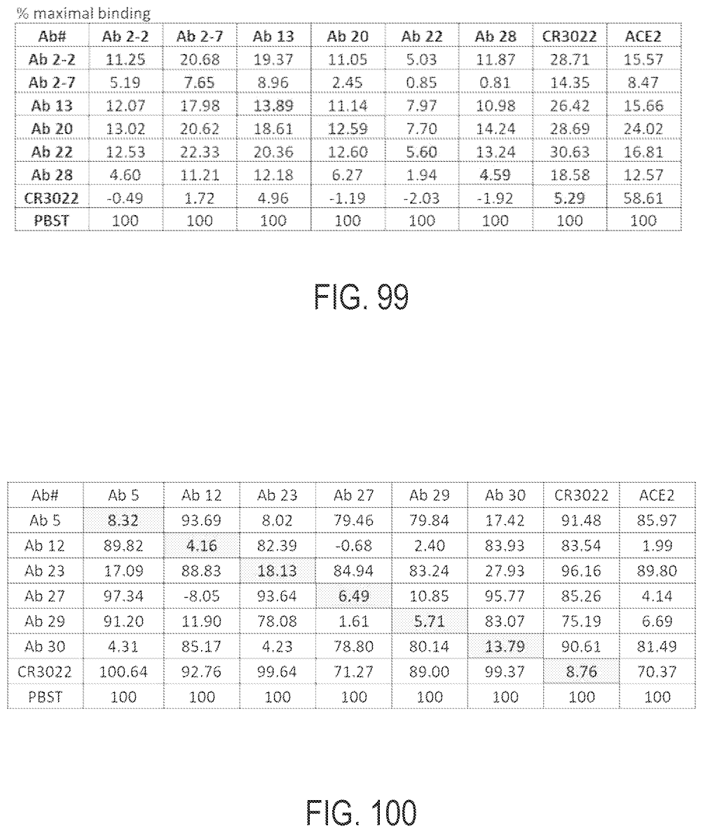

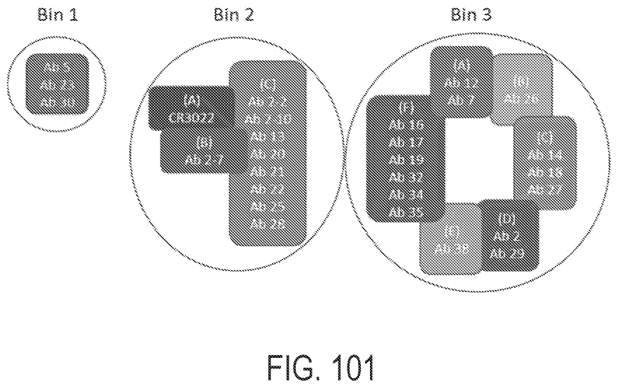

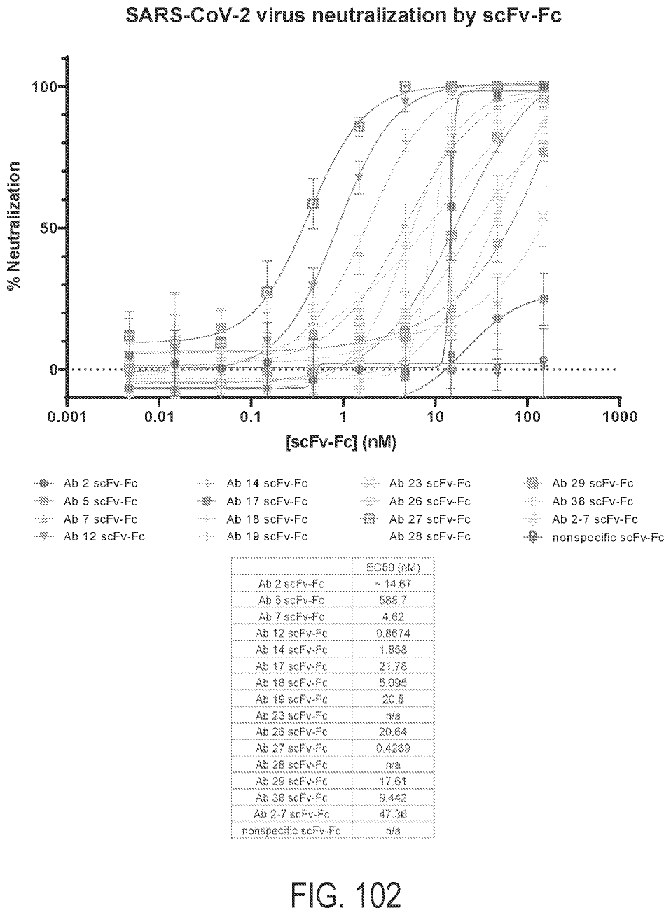

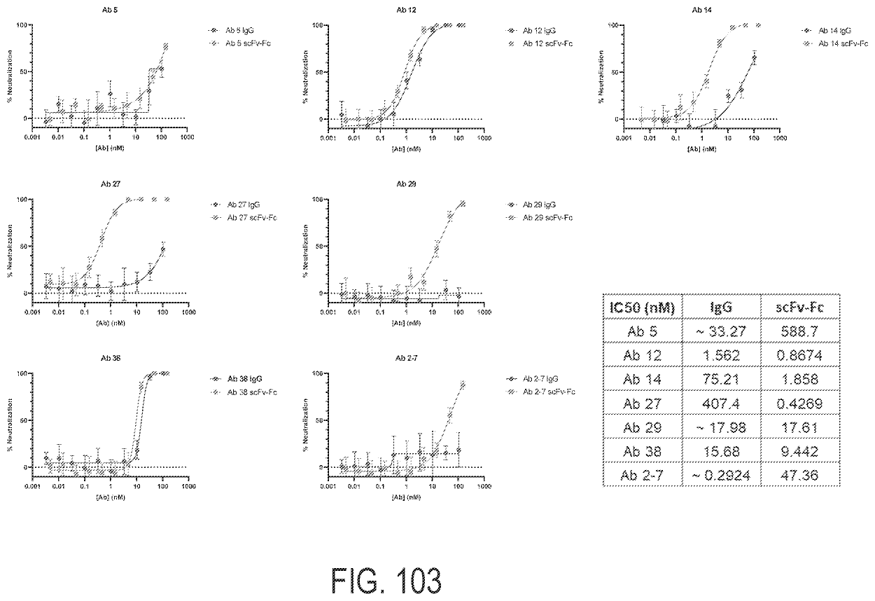

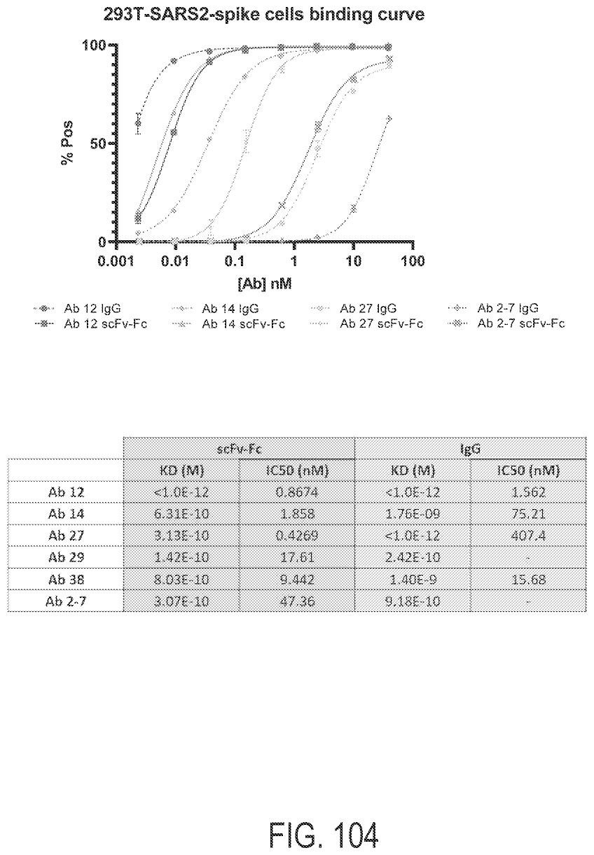

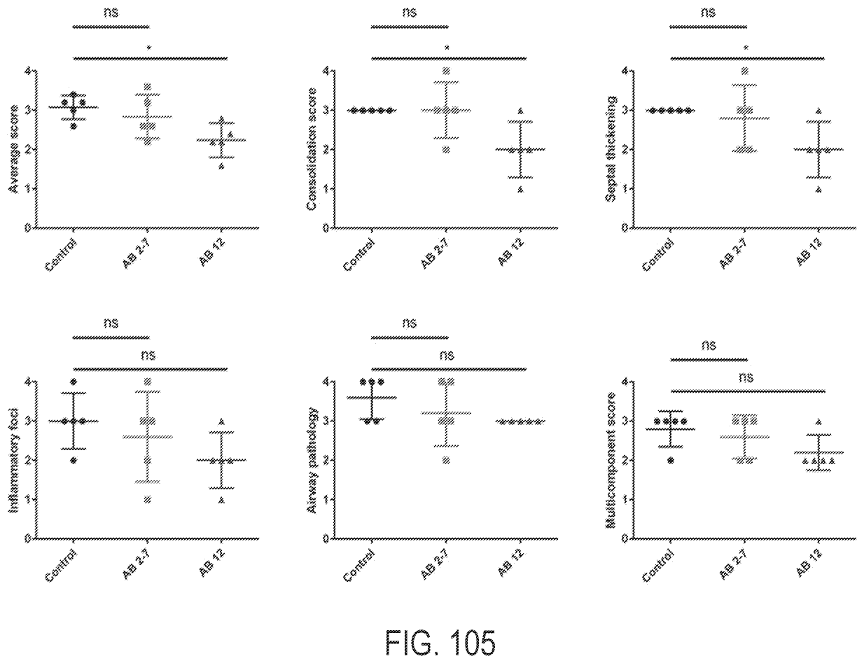

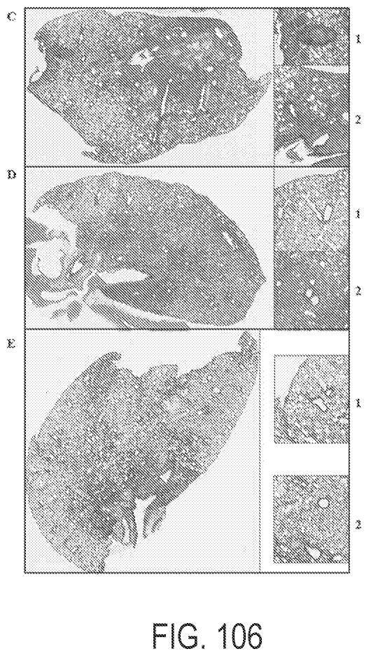

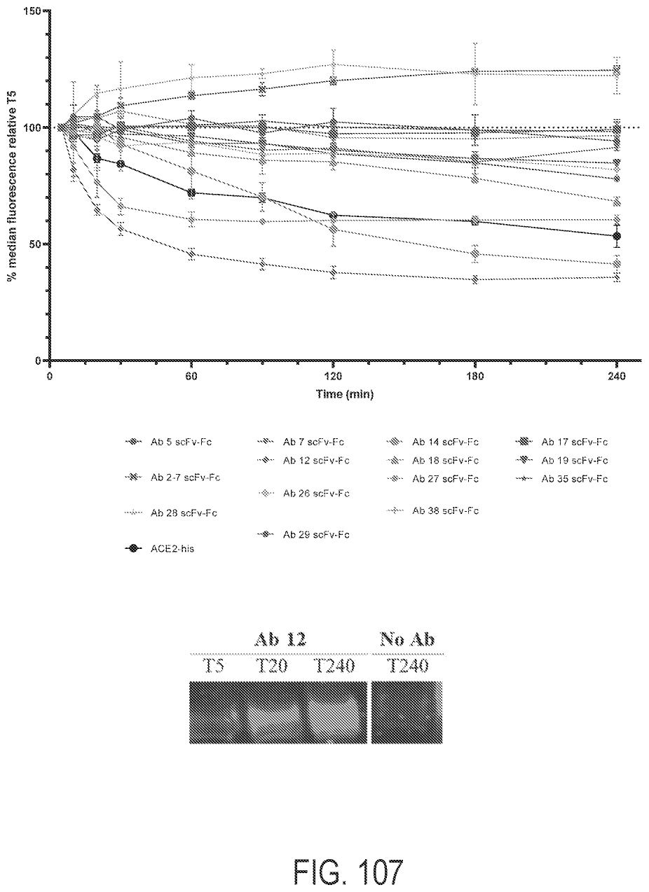

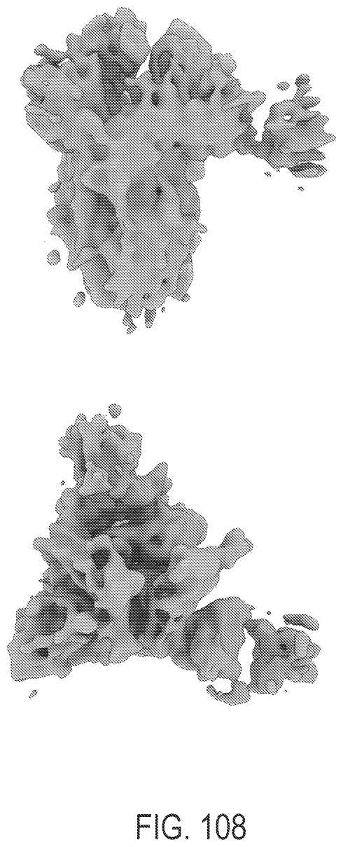

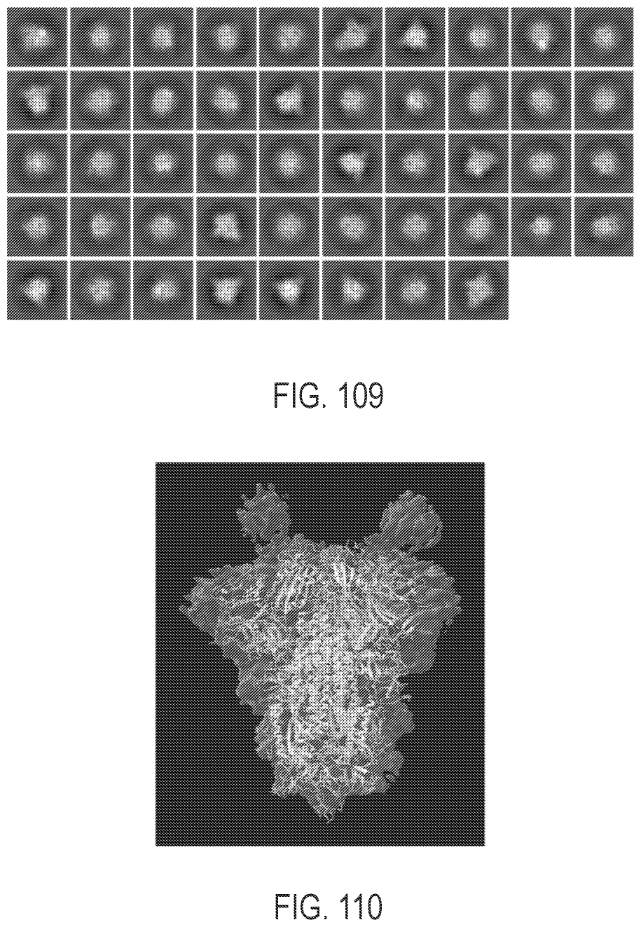

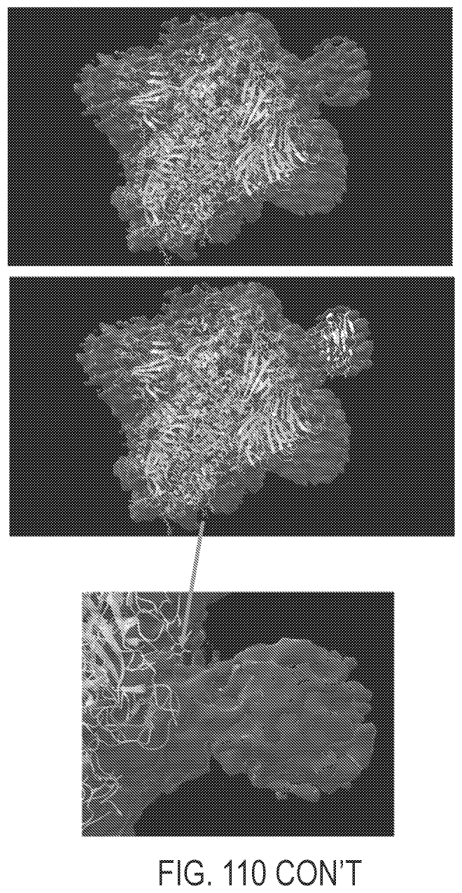

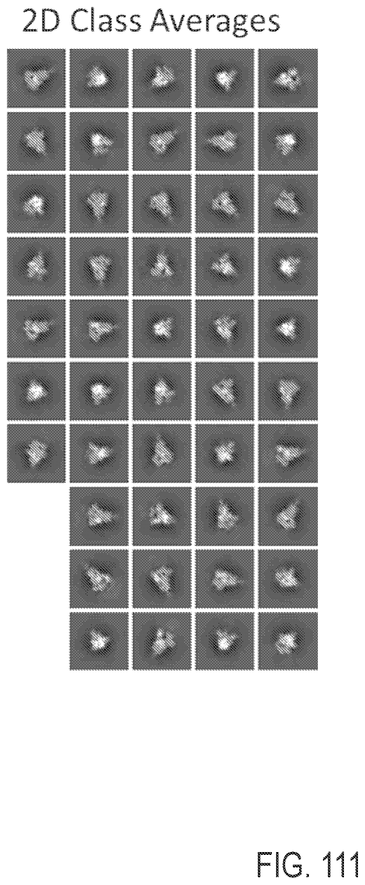

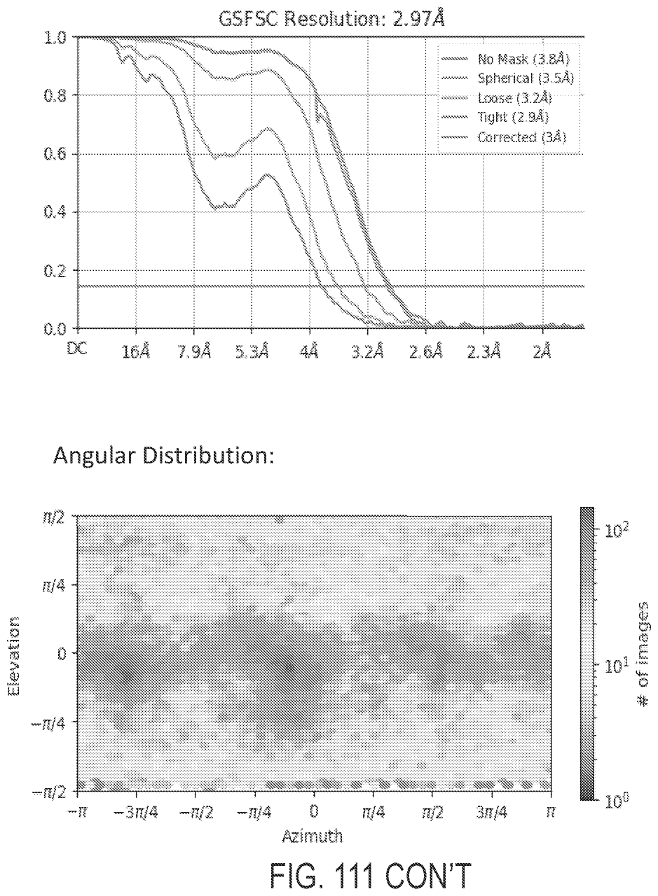

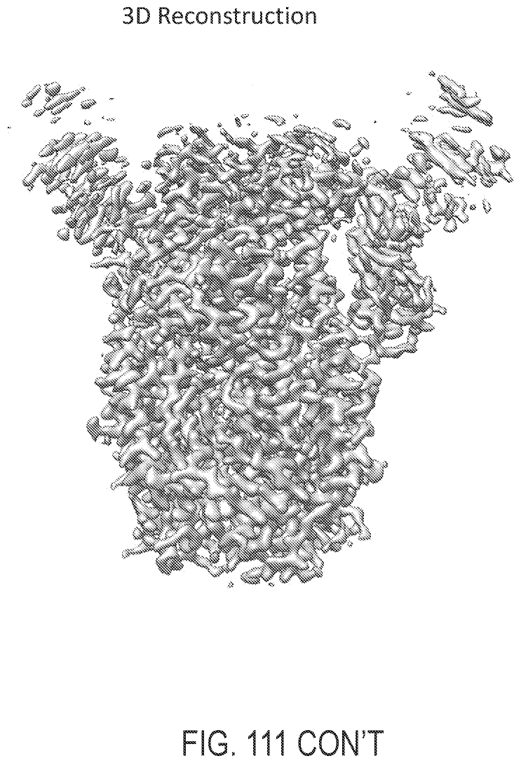

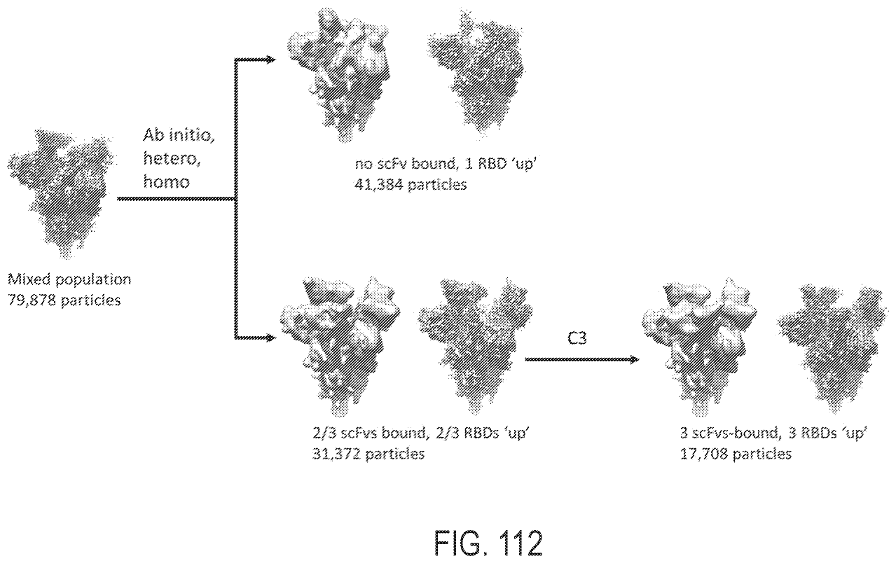

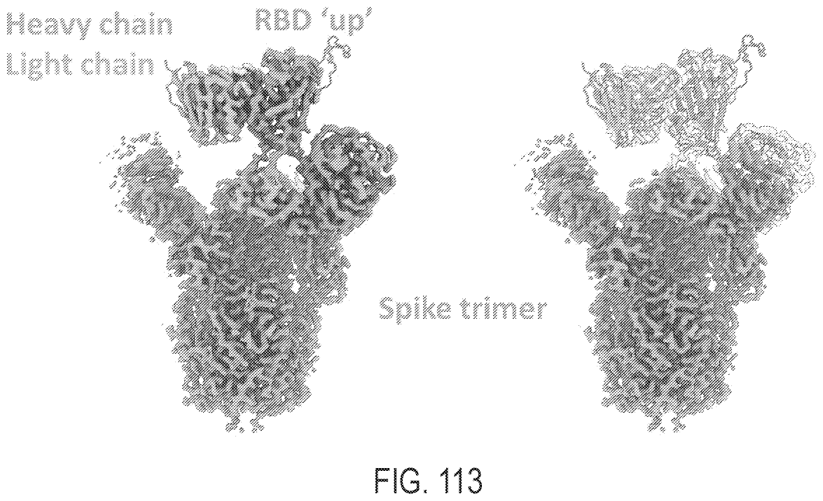

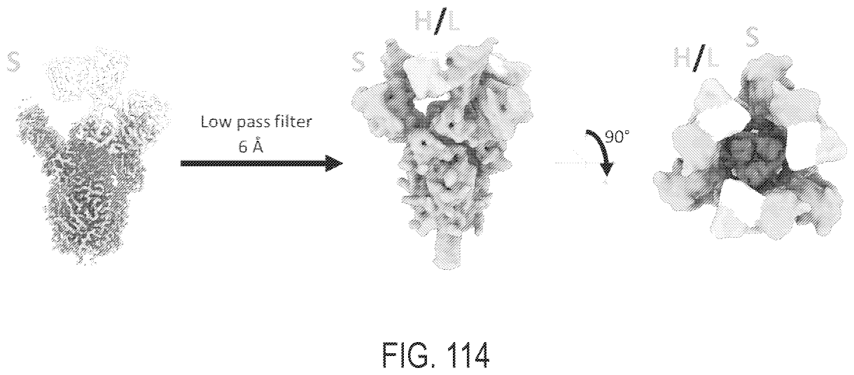

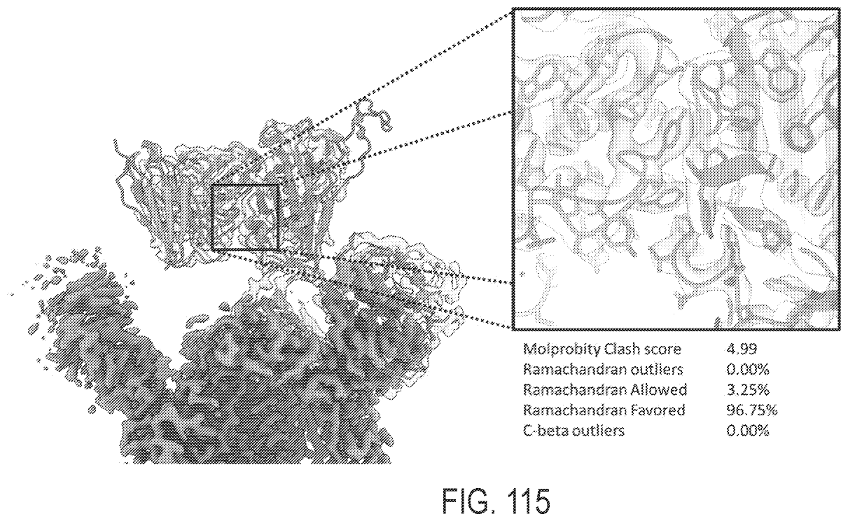

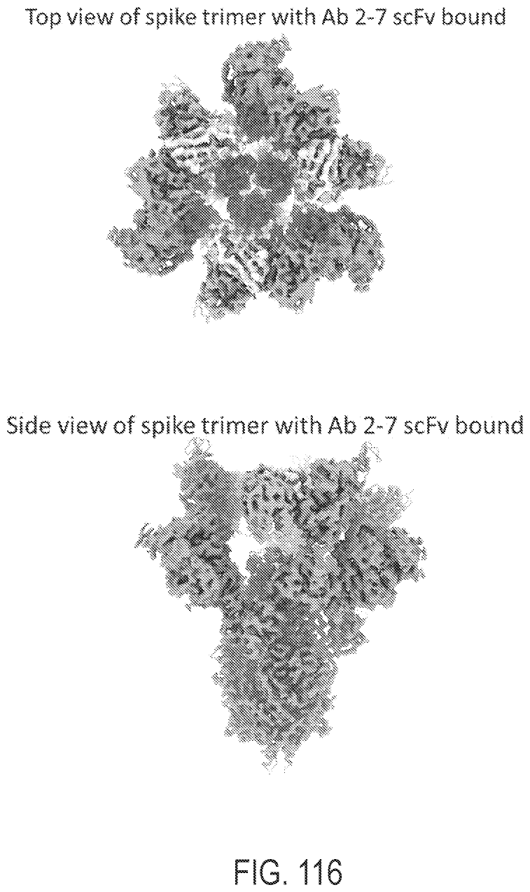

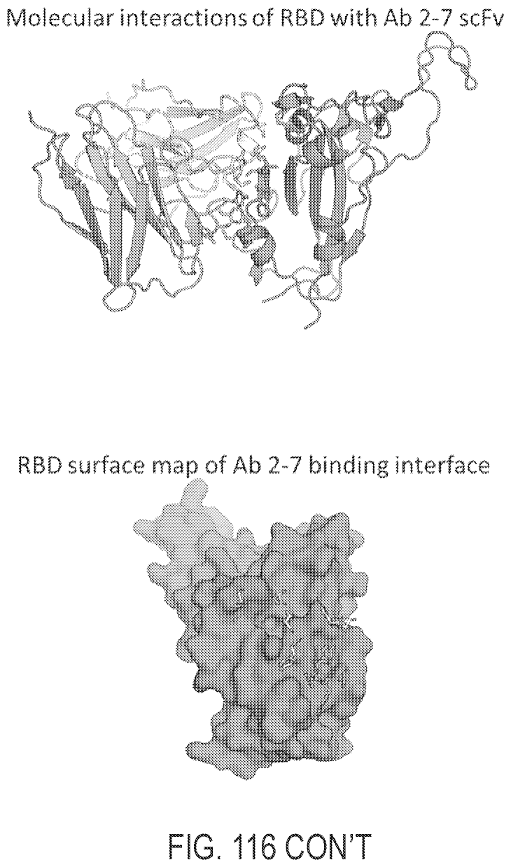

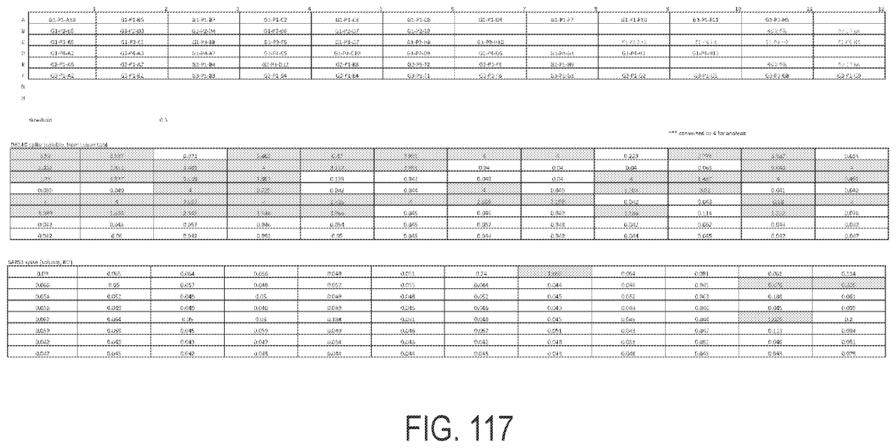

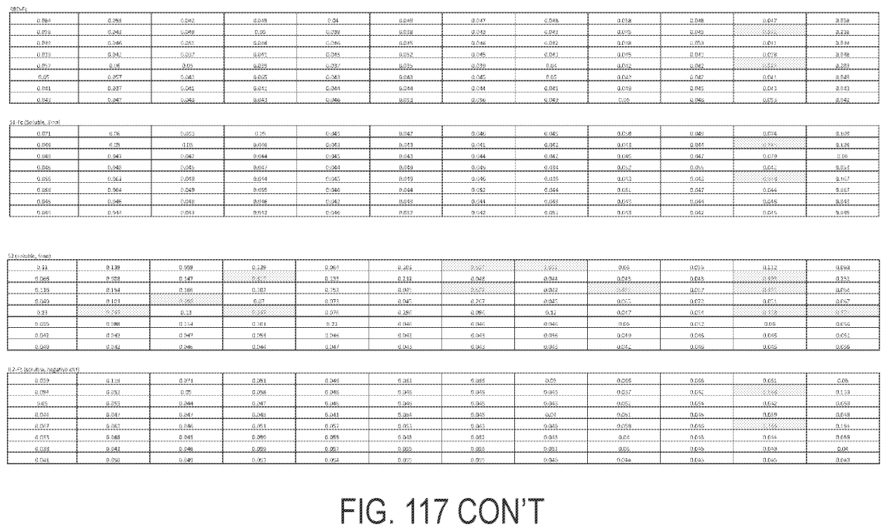

Pseudovirus neutralization was performed with SARS-2 spike pseudovirus and 293T-ACE2 transduced cells. As shown in FIG. 55, a number of neutralizing antibodies were identified with Ab 12 and Ab 27 being the most potent.

Kinetic data for both of these abs reveal tight binding antibodies with minimal disassociation. Additionally, epitope mapping reveals that they have similar but slightly different competition patterns. Ab 12 successfully competes with Abs 14, 15, 19, 26, and 27. While Ab 27 also competes with Abs 12, 14, and 15, it does not compete with Ab 19 or Ab 26. This indicates that they bind similar epitopes but might have a different angle of approach.

### Example 5—Live Virus Neutralization

Monoclonal antibodies were diluted to 100 ug/ml by adding 50 μl of 1 mg/ml antibody to 450 μl Dulbecco's Phosphate Buffered Saline (DPBS)(Gibco™). A series of 10 half-log dilutions was then prepared in triplicate for each antibody in DPBS. Each dilution was incubated at 37° C. and 5% C02 for 1 hour with 1000 plaque forming units/ml (PFU/ml) of SARS-CoV-2 (isolate USA-WA1/2020), diluted in Dulbecco's Modified Eagle Medium (DMEM) (Gibco™) containing 2% fetal bovine serum (Gibco™) and antibiotic-antimycotic (Gibco™). Controls included DMEM containing 2% fetal bovine serum and antibiotic-antimycotic only as a negative control, 1000 PFU/ml SARS-CoV-2 incubated with DPBS, and 1000 PFU/ml SARS-CoV-2 incubated with DMEM. Two hundred microliters of each dilution or control were added to confluent monolayers of NR-596 Vero E6 cells in triplicate and incubated for 1 hour at 37° C. and 5% C02. The plates were gently rocked every 5-10 minutes to prevent monolayer drying. The monolayers were then overlaid with a 1:1 mixture of 2.5% Avicel® RC-591 microcrystalline cellulose and carboxymethylcellulose sodium (DuPont Nutrition & Biosciences, Wilmington, DE) and 2×Modified Eagle Medium (Temin's modification, Gibco™) supplemented with 2×antibiotic-antimycotic (Gibco™), 2×GlutaMAX (Gibco™) and 10% fetal bovine serum (Gibco™). Plates were incubated at 37° C. and 5% CO² for 2 days. The monolayers were fixed with 10% neutral buffered formalin and stained with 0.2% aqueous Gentian Violet (RICCA Chemicals, Arlington, TX) in 10% neutral buffered formalin for 30 min, followed by rinsing and plaque counting. The half maximal inhibitory concentrations (IC50) were calculated using GraphPad Prism 8.

TABLE I
IC₅₀ values for monoclonal antibody neutralization
activity against SARS-CoV-2.

| | | | 95% Confidence |
|---|---|---|---|
| Antibody | Series | IC₅₀ (μg/ml) | Interval |
| Ab-12 | 1 | 0.09022 | 0.07418-0.1096 |
| | 2 | 0.1035 | 0.07667-0.1387 |
| | 3 | 0.08541 | 0.07014-0.1034 |
| Ab-14 | 1 | 0.1443 | 0.08394-0.2362 |
| | 2 | 0.2298 | 0.1977-0.2673 |
| | 3 | 0.2055 | 0.1467-0.2841 |
| Ab-19 | 1 | NA | NA |
| | 2 | NA | NA |
| | 3 | 0.9627 | 0.5487-4.851 |
| Ab-27 | 1 | 0.04904 | 0.03376-0.07059 |
| | 2 | 0.04447 | 0.03555-0.05574 |
| | 3 | 0.04227 | 0.03366-0.05350 |
| Ab-28 | 1 | NA | NA |
| | 2 | NA | NA |
| | 3 | NA | NA |

### Example 6—Pseudovirus Neutralization

Pseudovirus was made my transfecting LentiX cells with CMV-d8.2, HIV-luc, and pcDNA3.4-SARS2-spike-gp41 tail with lipofectamine 3000. The cells were incubated at 37° C. for 3 days before harvest and filtration (0.45 μm). Pseudovirus is either stored at 4° C. or used immediately.

Target cells: 293T-ACE2 transduced cells, seeded 10,000 cells/well in 100 μl day before.

For neutralization assay, 120 μl pseudovirus was incubated with 120 μl ab dilution at RT for 1 hour. Growth media was removed from the plate and replaced with 60 μl pseudovirus/Ab mixture (done in triplicate). The plates were incubated at 37° C. for 48 hours before the cells were lysed with Promega passive lysis buffer followed by luciferase measurement via Promega Bio-Glow.

Plate 2: single dilution at 100 μg/ml scFv-Fc for all 28 antibodies

Plate 4: titration curves of scFv-Fcs from set 1 (Ab 7, Ab 12, Ab2-2, Ab 2-7, Ab2-10)

Plate 6: titration curves of scFv-Fcs from set 2 (Ab 14, Ab 19, Ab 23, Ab 26, Ab 27, Ab 28)
*antibodies from second set were chosen based on competition assay, best binder was chosen for each bin

### Example 7—Thermal Shift Assay

Thermal shift assays (or differential scanning calorimetry/DSC) is used to measure the unfolding of a protein in real time via hydrophobic interactions. Protein is incubated in the presence of a fluorescent dye (SYPRO Orange) and in the folded state, there is low binding of the dye to the protein. As the temperature of the sample increases, the protein will unfold, gradually exposing the hydrophobic core of the protein to the SYPRO Orange in solution increasing the fluorescent signal. SYPRO orange fluorescence is quenched by H2O, hence there is lower fluorescence with a folded protein. (See Huynh, Kathy, and Carrie L Partch. “Analysis of protein stability and ligand interactions by thermal shift assay.” Current protocols in protein science vol. 79 28.9.1-28.9.14. 2 Feb. 2015, doi:10.1002/0471140864.ps2809s79; King, Amy C et al. “High-throughput measurement, correlation analysis, and machine-learning predictions for pH and thermal stabilities of Pfizer-generated antibodies.” Protein science: a publication of the Protein Society vol. 20, 9 (2011): 1546-57. doi:10.1002/pro.680).

### Example 8—Live Virus Neutralization Data

Monoclonal antibodies were diluted to 100 ug/ml by adding 50 μl of 1 mg/ml antibody to 450 μl Dulbecco's Phosphate Buffered Saline (DPBS)(Gibco™). A series of 10 half-log dilutions was then prepared in triplicate for each antibody in DPBS. Each dilution was incubated at 37° C. and 5% C02 for 1 hour with 1000 plaque forming units/ml (PFU/ml) of SARS-CoV-2 (isolate USA-WA1/2020), diluted in Dulbecco's Modified Eagle Medium (DMEM) (Gibco™) containing 2% fetal bovine serum (Gibco™) and antibiotic-antimycotic (Gibco™). Controls included DMEM containing 2% fetal bovine serum and antibiotic-antimycotic only as a negative control, 1000 PFU/ml SARS-CoV-2 incubated with DPBS, and 1000 PFU/ml SARS-CoV-2 incubated with DMEM. Two hundred microliters of each dilution or control were added to confluent monolayers of NR-596 Vero E6 cells in triplicate and incubated for 1 hour at 37° C. and 5% C02. The plates were gently rocked every 5-10 minutes to prevent monolayer drying. The monolayers were then overlaid with a 1:1 mixture of 2.5% Avicel® RC-591 microcrystalline cellulose and carboxymethylcellulose sodium (DuPont Nutrition & Biosciences, Wilmington, DE) and 2×Modified Eagle Medium (Temin's modification, Gibco™) supplemented with 2×antibiotic-antimycotic (Gibco™), 2×GlutaMAX (Gibco™) and 10% fetal bovine serum (Gibco™). Plates were incubated at 37° C. and 5% CO² for 2 days. The monolayers were fixed with 10% neutral buffered formalin and stained with 0.2% aqueous Gentian Violet (RICCA Chemicals, Arlington, TX) in 10% neutral buffered formalin for 30 min, followed by rinsing and plaque counting. The half maximal inhibitory concentrations (IC50) were calculated using GraphPad Prism 8.

### Example 9—Lung Lesion Analyses

Tissues originated from Syrian golden hamsters infected with SARS-CoV-2 and either treated with monoclonal antibodies or left untreated. Photographs of whole plucks (lungs and heart) taken at necropsy were provided. One H&E stained slide was presented for each animal. All tissues examined were fixed in formalin for at least 96 hours prior to preparation. Embedding, slide preparation and staining were conducted per standard protocol. Only lung tissue was presented for examination. Lung consolidation percentages were determined as a function of the total observed area affected by consolidation, defined as collapsed alveoli, infiltration of mononuclear inflammatory cells, and darkened (plum colored) staining. Infiltrated foci are regions with significant numbers of infiltrating inflammatory mononuclear cells. These are often readily identifiable as blue/purple patches in the tissue section. Infiltrated airways were defined as large or small airways fully or partly (>10%) occluded by mononuclear inflammatory cells.

Gross and clinical pathology findings: Patchy consolidation was observed on all lungs, with some apparent improvement in treated animals. However, it was difficult to assess the degree of consolidation, as it was difficult to differentiate consolidation from blood on the surface of the organ in the photographs provided.

Lung lesion score:
0: no lesions observed
1: 25% and under area of lesion coverage
2: 26%-49% area of lesion coverage
3: 50%-74% area of lesion coverage
4: 75% and above area of lesion coverage

TABLE II
Lung Lesion Scoring Table

| | Gross lung lesion | |
|---|---|---|
| ID# | score | Full Lung lobe appearance |
| Control 1 | 2 | Yes, rounded edges of lobes |
| Control 2 | 1 | Yes, rounded edges of lobes |
| Control 3 | 1 | Yes, rounded edges of lobes |
| Control 4 | 0 | Yes, rounded edges of lobes |
| Control 5 | 2 | Yes, rounded edges of lobes |
| A12 | 2 | No rounding of lobe edges |
| A12 | 1 | No rounding of lobe edges |
| A12 | 1 | No rounding of lobe edges |
| A12 | 2 | No rounding of lobe edges |
| A12 | 1 | No rounding of lobe edges |

Virus-Only:

General. Changes observed are consistent with viral interstitial pneumonia, namely alveolar wall thickening, alveolar collapse, and inflammatory cell infiltration. Airway obstruction by inflammatory cells common, and present in all sections.

| | % Infiltrated | No. of Infiltration | | |
|---|---|---|---|---|
| Animal | Airways | Foci | Consolidation | Comments |
| 1 | 45% | 8 | 13% | None. |
| 2 | 60% | 7 | 29% | None. |
| 3 | 25% | 3 | 56% | None. |
| 4 | 40% | 4 | 50% | None. |
| 5 | 55% | 5 | 20% | None. |

Tissues from untreated animals displayed gross pathology and histopathology consistent with viral interstitial pneumonia. Inflammatory cell infiltration present in all sections, along with infiltration of large and small airways and perivascular cuffing. Severity of pathology was variable between animals.

Lung tissues from animals that received monoclonal antibodies displayed effectively identical pathology, though certain sections had notable infiltration of inflammatory cells into large airways. Pathology was moderately variable between animals.

Consolidation and airway infiltration were significantly improved in animals treated with AB12 (p<0.05, Mann-Whitney test). No improvement was found in the number of observed foci of inflammatory infiltration.

Antibody 12:

General. Signs of typical histopathology associated with viral interstitial pneumonia (discussed previously) noted in all sections. Significantly improved consolidation relative to untreated animals. Some sections had notable infiltration of inflammatory cells into large airways.

| | % Infiltrated | No. of Infiltration | | |
|---|---|---|---|---|
| Animal | Airways | Foci | Consolidation | Comments |
| 1 | 10% | 1 | 0% | None. |
| 2 | 10% | 3 | 9% | None. |
| 3 | 30% | 4 | 8% | ** |
| 4 | 20% | 4 | 14% | None. |
| 5 | 40% | 5 | 3% | None. |

** Large airways with significant inflammatory cell infiltration noted

### Example 10—Syrian Golden Hamster Experiments

Syrian hamster SARS CoV virus challenge study. Animal challenge studies were conducted. 1 day before the challenge hamsters were microchipped. On day 0, hamsters were anesthetized with ketamine/xylazine and challenged with SARS-CoV-2 by the intranasal (IN) route with up to 10\hat{ }7 TCID50 (or 10\hat{ }6 PFU/ml) in a total volume up to 100 μL. The viral strain used is Wuhan Hu-1 strain, SARS-CoV strain 2019-nCoV/USA_WA1/2020 (WA1); GenBank: MN985325; GISAID: EPI_ISL_404895. For animal experiments passage 5 was used. The final challenge dose was 10000 plaque forming units diluted in sterile PBS. Body weight and body temperature were measured each day, starting at day 0. On day 1 post challenge (dpc) hamsters were treated with 5 mg/kg of monoclonal antibodies diluted in 0.5 ml of sterile PBS via intraperitoneal route (IP). The control group received an equal volume of sterile PBS via the same IP route. On day 3 post challenge all animals were sacrificed. At necropsy, terminal blood was collected into a labeled 3.5 mL SST vacutainer from all animals. Lungs were harvested for all groups.

Syrian golden hamster tissue processing and viral load determination. For the pathogenicity study, animals from each study group were euthanized on day 3 post challenge, and the lungs were harvested. Right lungs were placed in L15 medium supplemented with 10% fetal bovine serum (Gibco) and Antibiotic-Antimycotic solution (Gibco), flash-frozen in dry ice and stored at −80C until processing. Tissues were thawed and homogenized using the TissueLyser II system (Qiagen). Tissue homogenates were titrated on Vero E6 cell monolayers in 96-well plates to determine viral loads. 10× fold dilutions of the lung supematants were incubated for 1 hour and replaced with 100 μLs of 0.9% methylcellulose in minimal essential medium (MEM) containing 10% fetal bovine serum (Quality Biologicals) and 0.1% gentamicin sulfate (Mediatech), followed by incubation at 37 C. Plates were fixed with 10% buffered formalin (Thermo Fisher) with subsequent removal from the biocontainment laboratories. Foci were visualized by staining monolayers with a mixture of 37 SARS-CoV-2 specific human antibodies kindly provided by Distributed Bio. As the secondary antibody, HRP-labeled goat anti-human IgG (SeraCare) was used at dilution 1:500. Primary and secondary antibodies were diluted in 1×DPBS with 5% milk. Plaques were revealed by AEC substrate (enQuire Bioreagents).

Syrian golden hamster histopathology. During necropsy, gross lesions were noted and representative lung tissues from the left lobe were collected in 10% formalin. After a 24-hour initial fixation at 4C, the lung tissues were transferred to fresh 10% formalin for an additional 48-hour fixation, prior to removal from containment. Formalin-fixed tissues were processed by standard histological procedures by the UTMB Anatomic Pathology Core. About 4 μm-thick sections were cut and stained with hematoxylin and eosin (HE). Sections of lungs were examined for the extent of inflammation, type of inflammatory foci, and changes in alveoli/alveolar septa/airways/blood vessels in parallel with sections from uninfected or control animals. The blinded tissue sections were semi-quantitatively scored for pathological lesions using the criteria described in Table S1 (51). All slides were scored by a trained staff member. Significance was assessed using a Kruskall-Wallis test with Dunn's post-hoc correction.

TABLE III
Lung Lesion Scores

| | Gross lung lesion | |
|---|---|---|
| ID# | score | Full Lung lobe appearance |
| Control 1 | 2 | Yes, rounded edges of lobes |
| Control 2 | 1 | Yes, rounded edges of lobes |
| Control 3 | 1 | Yes, rounded edges of lobes |
| Control 4 | 0 | Yes, rounded edges of lobes |
| Control 5 | 2 | Yes, rounded edges of lobes |
| A12 | 2 | No rounding of lobe edges |
| A12 | 1 | No rounding of lobe edges |
| A12 | 1 | No rounding of lobe edges |
| A12 | 2 | No rounding of lobe edges |
| A12 | 1 | No rounding of lobe edges |

0: no lesions observed; 1: 25% and under area of lesion coverage; 2: 26%-49% area of lesion coverage; 3: 50%-74% area of lesion coverage; 4: 75% and above area of lesion coverage.

### Example 11—Spike Mutant Binding Studies

The table below shows examples of spike mutant binding to the SARS-CoV-2 antibodies described herein.

| | | Loading | | Loading | | | | |
|---|---|---|---|---|---|---|---|---|
| Sample | Dissoc. | Well | Loading | Conc. | | Conc. | | KD |
| ID | Loc. | Location | Sample ID | (μg/ml) | Cycle | (nM) | Response | (M) |
| WT Spike | p1A12 | A1 | Ab 2 scFv-Fc | 1 | 1 | 48.3 | 0.0652 | 1.99E−10 |
| WT Spike | p1A12 | A2 | Ab 5 scFv-Fc | 1 | 2 | 48.3 | 0.0828 | 6.31E−12 |
| WT Spike | p1A12 | A3 | Ab 7 scFv-Fc | 1 | 3 | 48.3 | 0.2424 | 7.00E−12 |
| WT Spike | p1A12 | A4 | Ab 8 scFv-Fc | 1 | 4 | 48.3 | 0.118 | 7.15E−12 |
| D614G | p1B12 | B1 | Ab 2 scFv-Fc | 1 | 1 | 48.3 | 0.1499 | 1.67E−10 |
| Spike | | | | | | | | |
| D614G | p1B12 | B2 | Ab 5 scFv-Fc | 1 | 2 | 48.3 | 0.1913 | 3.14E−12 |
| Spike | | | | | | | | |
| D614G | p1B12 | B3 | Ab 7 scFv-Fc | 1 | 3 | 48.3 | 0.345 | 2.74E−12 |
| Spike | | | | | | | | |
| D614G | p1B12 | B4 | Ab 8 scFv-Fc | 1 | 4 | 48.3 | 0.1871 | 7.30E−12 |
| Spike | | | | | | | | |
| B.1.1.7 | p1C12 | C1 | Ab 2 scFv-Fc | 1 | 1 | 48.4 | 0.1608 | 6.57E−10 |
| Spike | | | | | | | | |
| B.1.1.7 | p1C12 | C2 | Ab 5 scFv-Fc | 1 | 2 | 48.4 | 0.185 | 3.26E−10 |
| Spike | | | | | | | | |
| B.1.1.7 | p1C12 | C3 | Ab 7 scFv-Fc | 1 | 3 | 48.4 | 0.3565 | 1.10E−12 |
| Spike | | | | | | | | |
| B.1.1.7 | p1C12 | C4 | Ab 8 scFv-Fc | 1 | 4 | 48.4 | 0.2023 | 2.29E−12 |
| Spike | | | | | | | | |
| B.1.351 | p1D12 | D1 | Ab 2 scFv-Fc | 1 | 1 | 48.5 | 0.0439 | 5.03E−10 |
| Spike | | | | | | | | |
| B.1.351 | p1D12 | D2 | Ab 5 scFv-Fc | 1 | 2 | 48.5 | 0.1247 | 1.81E−11 |
| Spike | | | | | | | | |
| B.1.351 | p1D12 | D3 | Ab 7 scFv-Fc | 1 | 3 | 48.5 | 0.2329 | 1.67E−12 |
| Spike | | | | | | | | |
| B.1.351 | p1D12 | D4 | Ab 8 scFv-Fc | 1 | 4 | 48.5 | 0.0872 | 1.09E−09 |
| Spike | | | | | | | | |
| P.1 Spike | p1F12 | F1 | Ab 2 scFv-Fc | 1 | 1 | 48.3 | 0.0281 | 9.13E−12 |
| P.1 Spike | p1F12 | F2 | Ab 5 scFv-Fc | 1 | 2 | 48.3 | 0.1146 | 7.71E−12 |
| P.1 Spike | p1F12 | F3 | Ab 7 scFv-Fc | 1 | 3 | 48.3 | 0.242 | 1.09E−11 |
| P.1 Spike | p1F12 | F4 | Ab 8 scFv-Fc | 1 | 4 | 48.3 | 0.0784 | 1.06E−11 |
| WT Spike | p1A12 | A5 | Ab 2-2 scFv-Fc | 1 | 1 | 48.3 | 0.0747 | 9.01E−12 |
| WT Spike | p1A12 | A6 | Ab 2-7 scFv-Fc | 1 | 2 | 48.3 | 0.0672 | 7.85E−12 |
| WT Spike | p1A12 | A7 | Ab 2-10 scFv-Fc | 1 | 3 | 48.3 | 0.0423 | 5.65E−12 |
| WT Spike | p1A12 | A8 | Ab 12 scFv-Fc | 1 | 4 | 48.3 | 0.1857 | 8.31E−12 |
| WT Spike | p1A12 | A9 | Ab 13 scFv-Fc | 1 | 5 | 48.3 | 0.0732 | 8.55E−12 |
| D614G | p1B12 | B5 | Ab 2-2 scFv-Fc | 1 | 1 | 48.3 | 0.1324 | 8.91E−12 |
| Spike | | | | | | | | |
| D614G | p1B12 | B6 | Ab 2-7 scFv-Fc | 1 | 2 | 48.3 | 0.1161 | 9.93E−12 |
| Spike | | | | | | | | |
| D614G | p1B12 | B7 | Ab 2-10 scFv-Fc | 1 | 3 | 48.3 | 0.0723 | 8.87E−12 |
| Spike | | | | | | | | |
| D614G | p1B12 | B8 | Ab 12 scFv-Fc | 1 | 4 | 48.3 | 0.2182 | 1.12E−11 |
| Spike | | | | | | | | |
| D614G | p1B12 | B9 | Ab 13 scFv-Fc | 1 | 5 | 48.3 | 0.1071 | 8.10E−12 |
| Spike | | | | | | | | |
| B.1.1.7 | p1C12 | C5 | Ab 2-2 scFv-Fc | 1 | 1 | 48.4 | 0.1348 | 3.06E−12 |
| Spike | | | | | | | | |
| B.1.1.7 | p1C12 | C6 | Ab 2-7 scFv-Fc | 1 | 2 | 48.4 | 0.1272 | 3.79E−10 |
| Spike | | | | | | | | |
| B.1.1.7 | p1C12 | C7 | Ab 2-10 scFv-Fc | 1 | 3 | 48.4 | 0.0939 | 6.82E−10 |
| Spike | | | | | | | | |
| B.1.1.7 | p1C12 | C8 | Ab 12 scFv-Fc | 1 | 4 | 48.4 | 0.1773 | 2.73E−12 |
| Spike | | | | | | | | |
| B.1.1.7 | p1C12 | C9 | Ab 13 scFv-Fc | 1 | 5 | 48.4 | 0.1134 | 1.91E−12 |
| Spike | | | | | | | | |
| B.1.351 | p1D12 | D5 | Ab 2-2 scFv-Fc | 1 | 1 | 48.5 | 0.1185 | 6.53E−12 |
| Spike | | | | | | | | |
| B.1.351 | p1D12 | D6 | Ab 2-7 scFv-Fc | 1 | 2 | 48.5 | 0.1298 | 3.60E−10 |
| Spike | | | | | | | | |
| B.1.351 | p1D12 | D7 | Ab 2-10 scFv-Fc | 1 | 3 | 48.5 | 0.1032 | 5.23E−10 |
| Spike | | | | | | | | |
| B.1.351 | p1D12 | D8 | Ab 12 scFv-Fc | 1 | 4 | 48.5 | 0.0737 | 1.45E−09 |
| Spike | | | | | | | | |
| B.1.351 | p1D12 | D9 | Ab 13 scFv-Fc | 1 | 5 | 48.5 | 0.1027 | 7.35E−10 |
| Spike | | | | | | | | |
| P.1 Spike | p1F12 | F5 | Ab 2-2 scFv-Fc | 1 | 1 | 48.3 | 0.1434 | 8.94E−12 |
| P.1 Spike | p1F12 | F6 | Ab 2-7 scFv-Fc | 1 | 2 | 48.3 | 0.152 | 7.04E−12 |
| P.1 Spike | p1F12 | F7 | Ab 2-10 scFv-Fc | 1 | 3 | 48.3 | 0.0969 | 6.67E−12 |
| P.1 Spike | p1F12 | F8 | Ab 12 scFv-Fc | 1 | 4 | 48.3 | 0.0718 | 8.55E−12 |
| P.1 Spike | p1F12 | F9 | Ab 13 scFv-Fc | 1 | 5 | 48.3 | 0.1122 | 4.88E−12 |
| WT Spike | p1A12 | A1 | Ab 14 scFv-Fc | 1 | 1 | 48.3 | 0.3126 | 8.22E−12 |
| WT Spike | p1A12 | A2 | Ab 16 scFv-Fc | 1 | 2 | 48.3 | 0.346 | 1.18E−11 |
| WT Spike | p1A12 | A3 | Ab 17 scFv-Fc | 1 | 3 | 48.3 | 0.2478 | 6.35E−12 |
| WT Spike | p1A12 | A4 | Ab 18 scFv-Fc | 1 | 4 | 48.3 | 0.2165 | 9.07E−12 |
| D614G | p1B12 | B1 | Ab 14 scFv-Fc | 1 | 1 | 48.3 | 0.4015 | 8.12E−12 |
| Spike | | | | | | | | |
| D614G | p1B12 | B2 | Ab 16 scFv-Fc | 1 | 2 | 48.3 | 0.434 | 8.35E−12 |
| Spike | | | | | | | | |
| D614G | p1B12 | B3 | Ab 17 scFv-Fc | 1 | 3 | 48.3 | 0.3573 | 6.80E−12 |
| Spike | | | | | | | | |
| D614G | p1B12 | B4 | Ab 18 scFv-Fc | 1 | 4 | 48.3 | 0.2909 | 6.33E−12 |
| Spike | | | | | | | | |
| B.1.1.7 | p1C12 | C1 | Ab 14 scFv-Fc | 1 | 1 | 48.4 | 0.355 | 7.98E−12 |
| Spike | | | | | | | | |
| B.1.1.7 | p1C12 | C2 | Ab 16 scFv-Fc | 1 | 2 | 48.4 | 0.3876 | 2.36E−12 |
| Spike | | | | | | | | |
| B.1.1.7 | p1C12 | C3 | Ab 17 scFv-Fc | 1 | 3 | 48.4 | 0.3312 | 1.85E−12 |
| Spike | | | | | | | | |
| B.1.1.7 | p1C12 | C4 | Ab 18 scFv-Fc | 1 | 4 | 48.4 | 0.2559 | 9.28E−12 |
| Spike | | | | | | | | |
| B.1.351 | p1D12 | D1 | Ab 14 scFv-Fc | 1 | 1 | 48.5 | 0.2534 | 3.10E−12 |
| Spike | | | | | | | | |
| B.1.351 | p1D12 | D2 | Ab 16 scFv-Fc | 1 | 2 | 48.5 | 0.4045 | <1.0E−12 |
| Spike | | | | | | | | |
| B.1.351 | p1D12 | D3 | Ab 17 scFv-Fc | 1 | 3 | 48.5 | 0.1994 | 5.62E−10 |
| Spike | | | | | | | | |
| B.1.351 | p1D12 | D4 | Ab 18 scFv-Fc | 1 | 4 | 48.5 | 0.1204 | 1.34E−09 |
| Spike | | | | | | | | |
| P.1 Spike | p1F12 | F1 | Ab 14 scFv-Fc | 1 | 1 | 48.3 | 0.2699 | 9.75E−12 |
| P.1 Spike | p1F12 | F2 | Ab 16 scFv-Fc | 1 | 2 | 48.3 | 0.3497 | 7.93E−12 |
| P.1 Spike | p1F12 | F3 | Ab 17 scFv-Fc | 1 | 3 | 48.3 | 0.1772 | 8.00E−12 |
| P.1 Spike | p1F12 | F4 | Ab 18 scFv-Fc | 1 | 4 | 48.3 | 0.1088 | 2.97E−12 |
| WT Spike | p1A12 | A5 | Ab 19 scFv-Fc | 1 | 1 | 48.3 | 0.1214 | 8.90E−12 |
| WT Spike | p1A12 | A6 | Ab 20 scFv-Fc | 1 | 2 | 48.3 | 0.0985 | 9.01E−12 |
| WT Spike | p1A12 | A7 | Ab 21 scFv-Fc | 1 | 3 | 48.3 | 0.1473 | 9.25E−12 |
| WT Spike | p1A12 | A8 | Ab 22 scFv-Fc | 1 | 4 | 48.3 | 0.1001 | 8.87E−12 |
| WT Spike | p1A12 | A9 | Ab 23 scFv-Fc | 1 | 5 | 48.3 | 0.0845 | 8.84E−12 |
| D614G | p1B12 | B5 | Ab 19 scFv-Fc | 1 | 1 | 48.3 | 0.2371 | 1.09E−11 |
| Spike | | | | | | | | |
| D614G | p1B12 | B6 | Ab 20 scFv-Fc | 1 | 2 | 48.3 | 0.1899 | 8.64E−12 |
| Spike | | | | | | | | |
| D614G | p1B12 | B7 | Ab 21 scFv-Fc | 1 | 3 | 48.3 | 0.2275 | 8.00E−12 |
| Spike | | | | | | | | |
| D614G | p1B12 | B8 | Ab 22 scFv-Fc | 1 | 4 | 48.3 | 0.1713 | 8.89E−12 |
| Spike | | | | | | | | |
| D614G | p1B12 | B9 | Ab 23 scFv-Fc | 1 | 5 | 48.3 | 0.1623 | 8.82E−12 |
| Spike | | | | | | | | |
| B.1.1.7 | p1C12 | C5 | Ab 19 scFv-Fc | 1 | 1 | 48.4 | 0.2032 | 1.03E−11 |
| Spike | | | | | | | | |
| B.1.1.7 | p1C12 | C6 | Ab 20 scFv-Fc | 1 | 2 | 48.4 | 0.1733 | 2.60E−12 |
| Spike | | | | | | | | |
| B.1.1.7 | p1C12 | C7 | Ab 21 scFv-Fc | 1 | 3 | 48.4 | 0.1989 | 1.47E−12 |
| Spike | | | | | | | | |
| B.1.1.7 | p1C12 | C8 | Ab 22 scFv-Fc | 1 | 4 | 48.4 | 0.1485 | 9.13E−12 |
| Spike | | | | | | | | |
| B.1.1.7 | p1C12 | C9 | Ab 23 scFv-Fc | 1 | 5 | 48.4 | 0.1348 | 9.11E−12 |
| Spike | | | | | | | | |
| B.1.351 | p1D12 | D5 | Ab 19 scFv-Fc | 1 | 1 | 48.5 | 0.2584 | 1.18E−11 |
| Spike | | | | | | | | |
| B.1.351 | p1D12 | D6 | Ab 20 scFv-Fc | 1 | 2 | 48.5 | 0.2131 | 7.03E−12 |
| Spike | | | | | | | | |
| B.1.351 | p1D12 | D7 | Ab 21 scFv-Fc | 1 | 3 | 48.5 | 0.1483 | 3.69E−10 |
| Spike | | | | | | | | |
| B.1.351 | p1D12 | D8 | Ab 22 scFv-Fc | 1 | 4 | 48.5 | 0.1833 | 3.18E−12 |
| Spike | | | | | | | | |
| B.1.351 | p1D12 | D9 | Ab 23 scFv-Fc | 1 | 5 | 48.5 | 0.1892 | 7.58E−12 |
| Spike | | | | | | | | |
| P.1 Spike | p1F12 | F5 | Ab 19 scFv-Fc | 1 | 1 | 48.3 | 0.2044 | 8.94E−12 |
| P.1 Spike | p1F12 | F6 | Ab 20 scFv-Fc | 1 | 2 | 48.3 | 0.1695 | 1.12E−11 |
| P.1 Spike | p1F12 | F7 | Ab 21 scFv-Fc | 1 | 3 | 48.3 | 0.1031 | 9.52E−12 |
| P.1 Spike | p1F12 | F8 | Ab 22 scFv-Fc | 1 | 4 | 48.3 | 0.1363 | 8.85E−12 |
| P.1 Spike | p1F12 | F9 | Ab 23 scFv-Fc | 1 | 5 | 48.3 | 0.1228 | 8.79E−12 |
| WT Spike | p1A12 | A1 | Ab 25 scFv-Fc | 1 | 1 | 48.3 | 0.0219 | 8.90E−12 |
| WT Spike | p1A12 | A2 | Ab 26 scFv-Fc | 1 | 2 | 48.3 | 0.1853 | 1.10E−11 |
| WT Spike | p1A12 | A3 | Ab 27 scFv-Fc | 1 | 3 | 48.3 | 0.2015 | 8.78E−12 |
| WT Spike | p1A12 | A4 | Ab 28 scFv-Fc | 1 | 4 | 48.3 | 0.1318 | 8.90E−12 |
| D614G | p1B12 | B1 | Ab 25 scFv-Fc | 1 | 1 | 48.3 | 0.0425 | 8.56E−12 |
| Spike | | | | | | | | |
| D614G | p1B12 | B2 | Ab 26 scFv-Fc | 1 | 2 | 48.3 | 0.3036 | 9.97E−12 |
| Spike | | | | | | | | |
| D614G | p1B12 | B3 | Ab 27 scFv-Fc | 1 | 3 | 48.3 | 0.2756 | 7.53E−12 |
| Spike | | | | | | | | |
| D614G | p1B12 | B4 | Ab 28 scFv-Fc | 1 | 4 | 48.3 | 0.1986 | 9.41E−12 |
| Spike | | | | | | | | |
| B.1.1.7 | p1C12 | C1 | Ab 25 scFv-Fc | 1 | 1 | 48.4 | 0.0668 | 4.27E−10 |
| Spike | | | | | | | | |
| B.1.1.7 | p1C12 | C2 | Ab 26 scFv-Fc | 1 | 2 | 48.4 | 0.3357 | 1.37E−12 |
| Spike | | | | | | | | |
| B.1.1.7 | p1C12 | C3 | Ab 27 scFv-Fc | 1 | 3 | 48.4 | 0.2805 | 1.57E−12 |
| Spike | | | | | | | | |
| B.1.1.7 | p1C12 | C4 | Ab 28 scFv-Fc | 1 | 4 | 48.4 | 0.2057 | 1.49E−12 |
| Spike | | | | | | | | |
| B.1.351 | p1D12 | D1 | Ab 25 scFv-Fc | 1 | 1 | 48.5 | 0.0577 | 1.13E−12 |
| Spike | | | | | | | | |
| B.1.351 | p1D12 | D2 | Ab 26 scFv-Fc | 1 | 2 | 48.5 | 0.2143 | 1.78E−12 |
| Spike | | | | | | | | |
| B.1.351 | p1D12 | D3 | Ab 27 scFv-Fc | 1 | 3 | 48.5 | 0.0974 | 1.25E−12 |
| Spike | | | | | | | | |
| B.1.351 | p1D12 | D4 | Ab 28 scFv-Fc | 1 | 4 | 48.5 | 0.1197 | 6.38E−12 |
| Spike | | | | | | | | |
| P.1 Spike | p1F12 | F1 | Ab 25 scFv-Fc | 1 | 1 | 48.3 | 0.0396 | 8.72E−12 |
| P.1 Spike | p1F12 | F2 | Ab 26 scFv-Fc | 1 | 2 | 48.3 | 0.1877 | 1.03E−11 |
| P.1 Spike | p1F12 | F3 | Ab 27 scFv-Fc | 1 | 3 | 48.3 | 0.0795 | 8.90E−12 |
| P.1 Spike | p1F12 | F4 | Ab 28 scFv-Fc | 1 | 4 | 48.3 | 0.1023 | 8.91E−12 |
| WT Spike | p1A12 | A5 | Ab 29 scFv-Fc | 1 | 1 | 48.3 | 0.126 | 1.49E−10 |
| WT Spike | p1A12 | A6 | Ab 30 scFv-Fc | 1 | 2 | 48.3 | 0.1075 | 1.02E−12 |
| WT Spike | p1A12 | A7 | Ab 31 scFv-Fc | 1 | 3 | 48.3 | 0.1009 | <1.0E−12 |
| WT Spike | p1A12 | A8 | Ab 32 scFv-Fc | 1 | 4 | 48.3 | 0.1305 | 6.32E−12 |
| WT Spike | p1A12 | A9 | Ab 34 scFv-Fc | 1 | 5 | 48.3 | 0.1002 | 6.58E−12 |
| D614G | p1B12 | B5 | Ab 29 scFv-Fc | 1 | 1 | 48.3 | 0.2015 | 1.04E−12 |
| Spike | | | | | | | | |
| D614G | p1B12 | B6 | Ab 30 scFv-Fc | 1 | 2 | 48.3 | 0.1789 | 7.43E−12 |
| Spike | | | | | | | | |
| D614G | p1B12 | B7 | Ab 31 scFv-Fc | 1 | 3 | 48.3 | 0.1679 | 9.03E−12 |
| Spike | | | | | | | | |
| D614G | p1B12 | B8 | Ab 32 scFv-Fc | 1 | 4 | 48.3 | 0.2042 | 7.26E−12 |
| Spike | | | | | | | | |
| D614G | p1B12 | B9 | Ab 34 scFv-Fc | 1 | 5 | 48.3 | 0.19 | 9.15E−12 |
| Spike | | | | | | | | |
| B.1.1.7 | p1C12 | C5 | Ab 29 scFv-Fc | 1 | 1 | 48.4 | 0.1185 | 6.93E−10 |
| Spike | | | | | | | | |
| B.1.1.7 | p1C12 | C6 | Ab 30 scFv-Fc | 1 | 2 | 48.4 | 0.1389 | <1.0E−12 |
| Spike | | | | | | | | |
| B.1.1.7 | p1C12 | C7 | Ab 31 scFv-Fc | 1 | 3 | 48.4 | 0.1345 | <1.0E−12 |
| Spike | | | | | | | | |
| B.1.1.7 | p1C12 | C8 | Ab 32 scFv-Fc | 1 | 4 | 48.4 | 0.1506 | 2.02E−12 |
| Spike | | | | | | | | |
| B.1.1.7 | p1C12 | C9 | Ab 34 scFv-Fc | 1 | 5 | 48.4 | 0.1487 | 3.71E−12 |
| Spike | | | | | | | | |
| B.1.351 | p1D12 | D5 | Ab 29 scFv-Fc | 1 | 1 | 48.5 | 0.0418 | 3.71E−10 |
| Spike | | | | | | | | |
| B.1.351 | p1D12 | D6 | Ab 30 scFv-Fc | 1 | 2 | 48.5 | 0.0883 | <1.0E−12 |
| Spike | | | | | | | | |
| B.1.351 | p1D12 | D7 | Ab 31 scFv-Fc | 1 | 3 | 48.5 | 0.09 | <1.0E−12 |
| Spike | | | | | | | | |
| B.1.351 | p1D12 | D8 | Ab 32 scFv-Fc | 1 | 4 | 48.5 | 0.1152 | 7.11E−12 |
| Spike | | | | | | | | |
| B.1.351 | p1D12 | D9 | Ab 34 scFv-Fc | 1 | 5 | 48.5 | 0.1062 | 2.69E−12 |
| Spike | | | | | | | | |
| P.1 Spike | p1F12 | F5 | Ab 29 scFv-Fc | 1 | 1 | 48.3 | 0.0232 | 8.45E−12 |
| P.1 Spike | p1F12 | F6 | Ab 30 scFv-Fc | 1 | 2 | 48.3 | 0.0766 | 9.02E−12 |
| P.1 Spike | p1F12 | F7 | Ab 31 scFv-Fc | 1 | 3 | 48.3 | 0.0789 | 8.97E−12 |
| P.1 Spike | p1F12 | F8 | Ab 32 scFv-Fc | 1 | 4 | 48.3 | 0.1032 | 8.87E−12 |
| P.1 Spike | p1F12 | F9 | Ab 34 scFv-Fc | 1 | 5 | 48.3 | 0.0973 | 8.85E−12 |
| WT Spike | p1A12 | A1 | Ab 35 scFv-Fc | 1 | 1 | 48.3 | 0.1085 | 8.90E−12 |
| WT Spike | p1A12 | A2 | Ab 36 scFv-Fc | 1 | 2 | 48.3 | 0.076 | 9.01E−12 |
| WT Spike | p1A12 | A3 | Ab 37 scFv-Fc | 1 | 3 | 48.3 | 0.0801 | 9.00E−12 |
| WT Spike | p1A12 | A4 | Ab 38 scFv-Fc | 1 | 4 | 48.3 | 0.2736 | 7.86E−12 |
| WT Spike | p1A12 | A5 | Ab 39 scFv-Fc | 1 | 5 | 48.3 | 0.3695 | 2.04E−12 |
| D614G | p1B12 | B1 | Ab 35 scFv-Fc | 1 | 1 | 48.3 | 0.2332 | 8.69E−12 |
| Spike | | | | | | | | |
| D614G | p1B12 | B2 | Ab 36 scFv-Fc | 1 | 2 | 48.3 | 0.1619 | 7.42E−12 |
| Spike | | | | | | | | |
| D614G | p1B12 | B3 | Ab 37 scFv-Fc | 1 | 3 | 48.3 | 0.1568 | 8.54E−12 |
| Spike | | | | | | | | |
| D614G | p1B12 | B4 | Ab 38 scFv-Fc | 1 | 4 | 48.3 | 0.3275 | 3.37E−12 |
| Spike | | | | | | | | |
| D614G | p1B12 | B5 | Ab 39 scFv-Fc | 1 | 5 | 48.3 | 0.4581 | <1.0E−12 |
| Spike | | | | | | | | |
| B.1.1.7 | p1C12 | C1 | Ab 35 scFv-Fc | 1 | 1 | 48.4 | 0.1902 | 7.96E−12 |
| Spike | | | | | | | | |
| B.1.1.7 | p1C12 | C2 | Ab 36 scFv-Fc | 1 | 2 | 48.4 | 0.1462 | 5.16E−12 |
| Spike | | | | | | | | |
| B.1.1.7 | p1C12 | C3 | Ab 37 scFv-Fc | 1 | 3 | 48.4 | 0.1477 | 1.28E−12 |
| Spike | | | | | | | | |
| B.1.1.7 | p1C12 | C4 | Ab 38 scFv-Fc | 1 | 4 | 48.4 | 0.2667 | 1.47E−12 |
| Spike | | | | | | | | |
| B.1.1.7 | p1C12 | C5 | Ab 39 scFv-Fc | 1 | 5 | 48.4 | 0.2933 | <1.0E−12 |
| Spike | | | | | | | | |
| B.1.351 | p1D12 | D1 | Ab 35 scFv-Fc | 1 | 1 | 48.5 | 0.165 | 9.16E−12 |
| Spike | | | | | | | | |
| B.1.351 | p1D12 | D2 | Ab 36 scFv-Fc | 1 | 2 | 48.5 | 0.1342 | 8.07E−12 |
| Spike | | | | | | | | |
| B.1.351 | p1D12 | D3 | Ab 37 scFv-Fc | 1 | 3 | 48.5 | 0.1182 | 6.69E−12 |
| Spike | | | | | | | | |
| B.1.351 | p1D12 | D4 | Ab 38 scFv-Fc | 1 | 4 | 48.5 | 0.2343 | 7.62E−12 |
| Spike | | | | | | | | |
| B.1.351 | p1D12 | D5 | Ab 39 scFv-Fc | 1 | 5 | 48.5 | 0.1666 | 1.83E−12 |
| Spike | | | | | | | | |
| P.1 Spike | p1F12 | F1 | Ab 35 scFv-Fc | 1 | 1 | 48.3 | 0.1546 | 8.90E−12 |
| P.1 Spike | p1F12 | F2 | Ab 36 scFv-Fc | 1 | 2 | 48.3 | 0.1128 | 8.89E−12 |
| P.1 Spike | p1F12 | F3 | Ab 37 scFv-Fc | 1 | 3 | 48.3 | 0.1083 | 8.99E−12 |
| P.1 Spike | p1F12 | F4 | Ab 38 scFv-Fc | 1 | 4 | 48.3 | 0.2283 | 7.23E−12 |
| P.1 Spike | p1F12 | F5 | Ab 39 scFv-Fc | 1 | 5 | 48.3 | 0.1577 | 1.12E−11 |

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed:

1. An isolated monoclonal antibody, wherein the monoclonal antibody binds to an epitope in the receptor binding domain (RBD) of the spike protein (S) of a Severe Acute Respiratory Syndrome coronavirus (SARS-CoV2), and neutralizes SARS-CoV2.

2. The monoclonal antibody of claim 1, wherein the epitope is non-linear.

3. The monoclonal antibody of claim 1, wherein the epitope comprises a region within amino acids 319-490 of the spike protein (SEQ ID NO: 980).

4. The monoclonal antibody of claim 1, wherein the epitope comprises a region within amino acids 319-541 of the spike protein (SEQ ID NO: 980).

5. The monoclonal antibody of claim 1, wherein the monoclonal antibody inhibits viral and cell membrane fusion.

6. The monoclonal antibody of claim 1, wherein the monoclonal antibody competes with the binding of a monoclonal antibody to the spike protein.

7. The monoclonal antibody of claim 1, wherein the monoclonal antibody is a fully human antibody.

8. The monoclonal antibody of claim 1, wherein the monoclonal antibody blocks the binding of SARS-CoV2 spike protein to angiotensin converting enzyme 2 (ACE2) cell surface receptor.

9. The monoclonal antibody of claim 1, wherein the monoclonal antibody comprises:
a) a heavy chain with three CDRs comprising the amino acid sequences GGSIRTHS (SEQ ID NO:93), IHHSGAT (SEQ ID NO:94), and ARGPGILSY (SEQ ID NO:95) respectively, and/or a light chain with three CDRs comprising the amino acid sequences SSNIGSNT (SEQ ID NO:227), SNN (SEQ ID NO:228), and AAWDDSLNVHYV (SEQ ID NO:229) respectively;
b) a heavy chain with three CDRs comprising the amino acid sequences GGSISSYY (SEQ ID NO:96), IYTSGST (SEQ ID NO:97), and ARDVGFGWFDR (SEQ ID NO:98) respectively and/or a light chain with three CDRs comprising the amino acid sequences SGSIASNY (SEQ ID NO:230), EDN (SEQ ID NO:231), and QSFDSASLWV (SEQ ID NO:232) respectively;
c) a heavy chain with three CDRs comprising the amino acid sequences GGSIRTHS (SEQ ID NO:99), IHHSGAT (SEQ ID NO:100), and ARGPGILSY (SEQ ID NO:101) respectively and/or a light chain with three CDRs comprising the amino acid sequences SSNIGSND (SEQ ID NO:233), SNN (SEQ ID NO:234), and ATWDDSLSAGV (SEQ ID NO:235) respectively;
d) a heavy chain with three CDRs comprising the amino acid sequences GDSVSSYSDA (SEQ ID NO:102), TYYRSKWYN (SEQ ID NO:103), and AREIVATTPFRNYYYGMDV (SEQ ID NO: 104) respectively and/or a light chain with three CDRs comprising the amino acid sequences SGSIASNY (SEQ ID NO:236), QDK (SEQ ID NO:237), and QSYDSSSLWV (SEQ ID NO:238) respectively;
e) a heavy chain with three CDRs comprising the amino acid sequences GFTFSHYD (SEQ ID NO:105), IGYDGTNL (SEQ ID NO:106), and ARAANYYDSSGYGRADAFDI (SEQ ID NO:107) respectively and/or a light chain with three CDRs comprising the amino acid sequences TGSIAGNY (SEQ ID NO:239), DDN (SEQ ID NO:240), and QSYDSGNRGV (SEQ ID NO:241) respectively;
f) a heavy chain with three CDRs comprising the amino acid sequences GFTFSDFP (SEQ ID NO: 108), ISYDGNIK (SEQ ID NO:109), and A ARGGSSFDI (SEQ ID NO:110) respectively and/or a light chain with three CDRs comprising the amino acid sequences TSNIGNNA (SEQ ID NO:242), YNE (SEQ ID NO:243), and AAWDDSLSGHVV (SEQ ID NO:244) respectively;
g) a heavy chain with three CDRs comprising the amino acid sequences GFSLSTTGVG (SEQ ID NO:111), IYWNDDK (SEQ ID NO:112), and ARISGSGYFYPFDI (SEQ ID NO:113) respectively and/or a light chain with three CDRs comprising the amino acid sequences SGSIASNY (SEQ ID NO:245), EDN (SEQ ID NO:246), and QSYDSSSLWV (SEQ ID NO:247) respectively;
h) a heavy chain with three CDRs comprising the amino acid sequences GYTFSDYY (SEQ ID NO:120), IDPNSGGT (SEQ ID NO:121), and ARDRGRGGQAGAFDY (SEQ ID NO:978) respectively and/or a light chain with three CDRs comprising the amino acid sequences KIGSKS (SEQ ID NO:254), DDS (SEQ ID NO:255), and HVWDSSSDQNV (SEQ ID NO:256) respectively;
i) a heavy chain with three CDRs comprising the amino acid sequences GFTFSSYA (SEQ ID NO:122), ISYGGSNK (SEQ ID NO:123), and AKVRGSGWYWGSAFDI (SEQ ID NO:124) respectively and/or a light chain with three CDRs comprising the amino acid sequences SLRAYF (SEQ ID NO:257), GQD (SEQ ID NO:258), and NSRDSGENHLI (SEQ ID NO:259) respectively;
j) a heavy chain with three CDRs comprising the amino acid sequences GYSFTGSH (SEQ ID NO:125), INPDSGVI (SEQ ID NO:126), and ARDKAIGYVWALDY (SEQ ID NO:127) respectively and/or a light chain with three CDRs comprising the amino acid sequences SSDVGTYNR (SEQ ID NO:260), EVS (SEQ ID NO:261), and SSYTRTFTYV (SEQ ID NO:262) respectively;
k) a heavy chain with three CDRs comprising the amino acid sequences GVSLDTIGMR (SEQ ID NO:128), IDWDDDK (SEQ ID NO: 129), and ARSGLLYDLDV (SEQ ID NO: 130) respectively and/or a light chain with three CDRs comprising the amino acid sequences DSDIGANF (SEQ ID NO:263), RNT (SEQ ID NO:264), and QSYDSSLSAYV (SEQ ID NO:265) respectively;
l) a heavy chain with three CDRs comprising the amino acid sequences GYSFTSYW (SEQ ID NO:134), IYPGDSDT (SEQ ID NO:135), and ARGWQWHDY (SEQ ID NO:136) respectively and/or a light chain with three CDRs comprising the amino acid sequences SLRSYY (SEQ ID NO:269), DKD (SEQ ID NO:270), and NSRDRSDNHVV (SEQ ID NO:271) respectively;
m) a heavy chain with three CDRs comprising the amino acid sequences GDSVSSRSSA (SEQ ID NO:137), TYYRSNWNY (SEQ ID NO:138), and VRNMRPDFDL (SEQ ID NO: 139) respectively and/or a light chain with three CDRs comprising the amino acid sequences QSVSNN (SEQ ID NO:272), DAT (SEQ ID NO:273), and QQYDNLPV (SEQ ID NO:274) respectively;
n) a heavy chain with three CDRs comprising the amino acid sequences GYTFTTSG (SEQ ID NO: 140), ISAYNGNT (SEQ ID NO:141), and ARDFHLYYGMDV (SEQ ID NO:142) respectively and/or a light chain with three CDRs comprising the amino acid sequences SSDVGAYNY (SEQ ID NO:275), DVT (SEQ ID NO:276), and AVWDDGLNGRVV (SEQ ID NO:277) respectively;
o) a heavy chain with three CDRs comprising the amino acid sequences GGTFSSYA (SEQ ID NO:143), INPNSGGT (SEQ ID NO:144), and ARGSGGYYLG (SEQ ID NO:145) respectively and/or a light chain with three CDRs comprising the amino acid sequences SNNVGNQG (SEQ ID NO:278), MNN (SEQ ID NO:279), and SAWDSSLSRWV (SEQ ID NO:280) respectively;
p) a heavy chain with three CDRs comprising the amino acid sequences GGTFSSYT (SEQ ID NO:146), IIPILGTP (SEQ ID NO:147), and AVGSGWYSGFDY (SEQ ID NO:148) respectively and/or a light chain with three CDRs comprising the amino acid sequences SGSIASNY (SEQ ID NO:281), EDS (SEQ ID NO:282), and QSFHNSNPVI (SEQ ID NO:283) respectively;
q) a heavy chain with three CDRs comprising the amino acid sequences GFTFSSYW (SEQ ID NO:149), IKQDGSEK (SEQ ID NO:150), and ARGFYYYGAFDI (SEQ ID NO:151) respectively and/or a light chain with three CDRs comprising the amino acid sequences SGSIASNY (SEQ ID NO:284), EDN (SEQ ID NO:285), and QSYDSSNHWV (SEQ ID NO:286) respectively;
r) a heavy chain with three CDRs comprising the amino acid sequences GFTFDDYA (SEQ ID NO:152), IDWNSGVI (SEQ ID NO:153), and AKDAYSYGFLGAFDI (SEQ ID NO: 154) respectively and/or a light chain with three CDRs comprising the amino acid sequences NIGSKS (SEQ ID NO:287), EDR (SEQ ID NO:288), and QVWDGDSDHYV (SEQ ID NO:289) respectively;
s) a heavy chain with three CDRs comprising the amino acid sequences GFTFDDYA (SEQ ID NO:155), IDWNSGVI (SEQ ID NO:156), and ARDILPSNFDGKKIIVFQPPAKRDLDNYYGMDV (SEQ ID NO:157) respectively and/or a light chain with three CDRs comprising the amino acid sequences SSDVGGYNL (SEQ ID NO:290), EGS (SEQ ID NO:291), and SSYTITDVVV (SEQ ID NO:292) respectively; or
t) a heavy chain with three CDRs comprising the amino acid sequences GYSFTSNW (SEQ ID NO:158), IFPGDSDT (SEQ ID NO:159), and ARESYNAYGS (SEQ ID NO:160) respectively and/or a light chain with three CDRs comprising the amino acid sequences SSNIGSNP (SEQ ID NO:293), SNN (SEQ ID NO:294), and AAWDDSLSGVV (SEQ ID NO:295) respectively.

10. The monoclonal antibody of claim 1, wherein the monoclonal antibody comprises:
a) a heavy chain with three CDRs comprising the amino acid sequences GFTFTTYG (SEQ ID NO:114), ISYDGSIK (SEQ ID NO:115), and ARVGDSSSYYGIDA (SEQ ID NO:116) respectively, and/or a light chain with three CDRs comprising the amino acid sequences SSNIGSNS (SEQ ID NO:248), SNN (SEQ ID NO:249), and AAWDDSLTGYV (SEQ ID NO:250) respectively;
b) a heavy chain with three CDRs comprising the amino acid sequences GFTFSSHA (SEQ ID NO:117), ISYDGSYT (SEQ ID NO:118), and ARDWVNFGMDV (SEQ ID NO:119) respectively, and/or a light chain with three CDRs comprising the amino acid sequences SSDVGGYNY (SEQ ID NO:251), EVS (SEQ ID NO:252), and AAWDDSLSGPV (SEQ ID NO:253) respectively; or
c) a heavy chain with three CDRs comprising the amino acid sequences GFTFSDYP (SEQ ID NO:131), TSYDGRIK (SEQ ID NO:132), and ARDPGWLRSVGMDV (SEQ ID NO:133) respectively, and/or a light chain with three CDRs comprising the amino acid sequences SGSIARNY (SEQ ID NO:266), ADR (SEQ ID NO:267), and QSYDSSNQAAV (SEQ ID NO:268) respectively.

11. The monoclonal antibody of claim 1, wherein the monoclonal antibody comprises:
a) a heavy chain with three CDRs comprising the amino acid sequences GYTFTSYG (SEQ ID NO:161), ISAYNGNT (SEQ ID NO:162), and ARGFPQLGSDY (SEQ ID NO: 163) respectively, and/or a light chain with three CDRs comprising the amino acid sequences SGSIASNY (SEQ ID NO:296), EDN (SEQ ID NO:297), and QAWDSNSYV (SEQ ID NO:298) respectively;
b) a heavy chain with three CDRs comprising the amino acid sequences GGTFSSYA (SEQ ID NO:164), ISGYNGNT (SEQ ID NO:165), and ARQMKDSGNYWEYYYYGMDV (SEQ ID NO:166) respectively, and/or a light chain with three CDRs comprising the amino acid sequences NIGSES (SEQ ID NO:299), EDR (SEQ ID NO:300), and QVWNPSGSLQYV (SEQ ID NO:301) respectively;
c) a heavy chain with three CDRs comprising the amino acid sequences GYTFTSYG (SEQ ID NO:167), ISTYNGNT (SEQ ID NO:168), and ARDVFGHFDY (SEQ ID NO:169) respectively, and/or a light chain with three CDRs comprising the amino acid sequences SGNIATNY (SEQ ID NO:302), EDN (SEQ ID NO:303), and KSYDDGNHV (SEQ ID NO:304) respectively;
d) a heavy chain with three CDRs comprising the amino acid sequences GFSLTTTGVS (SEQ ID NO:170), IHWDDDK (SEQ ID NO:171), and ASFIMTVYAEYFED (SEQ ID NO: 172) respectively, and/or a light chain with three CDRs comprising the amino acid sequences QSVSSN (SEQ ID NO:305), DVS (SEQ ID NO:306), and QQRGVWPLT (SEQ ID NO:307) respectively;
e) a heavy chain with three CDRs comprising the amino acid sequences GFSLSTSAMC (SEQ ID NO:173), IDWDNDR (SEQ ID NO:174), and AHSPYDSIWGSFRPSVYYFDY (SEQ ID NO:175) respectively, and/or a light chain with three CDRs comprising the amino acid sequences SGSIVSSY (SEQ ID NO:308), EHN (SEQ ID NO:309), and QSYDSQNGV (SEQ ID NO:310) respectively;
f) a heavy chain with three CDRs comprising the amino acid sequences GFTFSDYY (SEQ ID NO:176), ISSSSSDT (SEQ ID NO:177), and AMPTREPAY (SEQ ID NO:178) respectively, and/or a light chain with three CDRs comprising the amino acid sequences SSDLGTYNY (SEQ ID NO:311), DVF (SEQ ID NO:312), and SSYTSSSTYV (SEQ ID NO:313) respectively;
g) a heavy chain with three CDRs comprising the amino acid sequences GFAFSDFP (SEQ ID NO: 179), ISYDGSLK (SEQ ID NO:180), and AREGVSNSRPFDH (SEQ ID NO:181) respectively, and/or a light chain with three CDRs comprising the amino acid sequences SIGTKS (SEQ ID NO:314), DDS (SEQ ID NO:315), and QVWESDDDDLV (SEQ ID NO:316) respectively;
h) a heavy chain with three CDRs comprising the amino acid sequences GFTFSSYA (SEQ ID NO:182), ISSNGGST (SEQ ID NO:183), and TRDLWSGSADSFDI (SEQ ID NO:184) respectively, and/or a light chain with three CDRs comprising the amino acid sequences SLRRYY (SEQ ID NO:317), GKN (SEQ ID NO:318), and NSRDISDNQWQWI (SEQ ID NO:319) respectively;
i) a heavy chain with three CDRs comprising the amino acid sequences GFPFNAYY (SEQ ID NO:185), INQDGSEK (SEQ ID NO:186), and ARLYWWGMDV (SEQ ID NO:187) respectively, and/or a light chain with three CDRs comprising the amino acid sequences SSDVGGYKY (SEQ ID NO:320), DVN (SEQ ID NO:321), and SSYTGRMNLYV (SEQ ID NO:322) respectively;
j) a heavy chain with three CDRs comprising the amino acid sequences GFTFDDYA (SEQ ID NO:188), IDWNSGVI (SEQ ID NO:189), and AKDAYSYGFLGAFDI (SEQ ID NO:190) respectively, and/or a light chain with three CDRs comprising the amino acid sequences NIRTKG (SEQ ID NO:323), YAS (SEQ ID NO:324), and QVWDSSSDLVV (SEQ ID NO:325) respectively;
k) a heavy chain with three CDRs comprising the amino acid sequences GFTFDDYA (SEQ ID NO:191), ISWNSGSI (SEQ ID NO:192), and ARDWWGSIDH (SEQ ID NO:193) respectively, and/or a light chain with three CDRs comprising the amino acid sequences SSDVGGYDY (SEQ ID NO:326), DVS (SEQ ID NO:327), and SSYTSSSPVV (SEQ ID NO:328) respectively;
l) a heavy chain with three CDRs comprising the amino acid sequences GGSISSSNW (SEQ ID NO:194), IYHSGST (SEQ ID NO:195), and ARRGGTYHRGAFDI (SEQ ID NO:196) respectively, and/or a light chain with three CDRs comprising the amino acid sequences SRDVGSYDL (SEQ ID NO:329), EGS (SEQ ID NO:330), and SSYTSSNSLV (SEQ ID NO:331) respectively;
m) a heavy chain with three CDRs comprising the amino acid sequences GASISNSF (SEQ ID NO:197), TSYSGNS (SEQ ID NO:198), and ARREWIKGHFDY (SEQ ID NO:199) respectively, and/or a light chain with three CDRs comprising the amino acid sequences GGSIASNY (SEQ ID NO:332), EDN (SEQ ID NO:333), and QSYDSSNPVV (SEQ ID NO:334) respectively;
n) a heavy chain with three CDRs comprising the amino acid sequences GGSFTTHS (SEQ ID NO:200), ILPGGAT (SEQ ID NO:201), and ARGPGILSY (SEQ ID NO:202) respectively, and/or a light chain with three CDRs comprising the amino acid sequences SSIGSND (SEQ ID NO:335), SNN (SEQ ID NO:336), and AWDDSLSAVV (SEQ ID NO:337) respectively;
o) a heavy chain with three CDRs comprising the amino acid sequences GGSFRTHS (SEQ ID NO:203), IHHSGAT (SEQ ID NO:204), and ARGPGILSY (SEQ ID NO:205) respectively, and/or a light chain with three CDRs comprising the amino acid sequences SSNIGSNT (SEQ ID NO:338), INN (SEQ ID NO:339), and AEWYDSLNVHYV (SEQ ID NO:340) respectively;
p) a heavy chain with three CDRs comprising the amino acid sequences GGSIRTHS (SEQ ID NO:206), IHHSGAT (SEQ ID NO:207), and ARGPGILSY (SEQ ID NO:208) respectively, and/or a light chain with three CDRs comprising the amino acid sequences SSNIGSNT (SEQ ID NO:341), INN (SEQ ID NO:342), and AECYDSLNDHYV (SEQ ID NO:343) respectively;
q) a heavy chain with three CDRs comprising the amino acid sequences GGSIRTHS (SEQ ID NO:209), IHHSGAT (SEQ ID NO:210), and GRGPGILSY (SEQ ID NO:211) respectively, and/or a light chain with three CDRs comprising the amino acid sequences SSNIGSNT (SEQ ID NO:344), SNN (SEQ ID NO:345), and AAWDDSLNVHYV (SEQ ID NO:346) respectively;
r) a heavy chain with three CDRs comprising the amino acid sequences GYSFTSYW (SEQ ID NO:212), IYPGDSDT (SEQ ID NO:213), and ARQGDGGGYDY (SEQ ID NO:214) respectively, and/or a light chain with three CDRs comprising the amino acid sequences SSNIGSNP (SEQ ID NO:347), NNN (SEQ ID NO:348), and AAWDDSLNGL (SEQ ID NO:349) respectively;
s) a heavy chain with three CDRs comprising the amino acid sequences RYSFSNYW (SEQ ID NO:215), IYPYDSDT (SEQ ID NO:216), and ARQGSSQSFDI (SEQ ID NO:217) respectively, and/or a light chain with three CDRs comprising the amino acid sequences SLRSYY (SEQ ID NO:350), QDS (SEQ ID NO:351), and QAWDSNSYV (SEQ ID NO:352) respectively;
t) a heavy chain with three CDRs comprising the amino acid sequences GYSFTSYW (SEQ ID NO:218), IYPGDSDT (SEQ ID NO:219), and ARRRGSAAAFDT (SEQ ID NO:220) respectively, and/or a light chain with three CDRs comprising the amino acid sequences SSNIGSNP (SEQ ID NO:353), DNN (SEQ ID NO:354), and EAWDDSLSGPV (SEQ ID NO:355) respectively;
u) a heavy chain with three CDRs comprising the amino acid sequences GYSFTSYW (SEQ ID NO:221), IYPGDSDT (SEQ ID NO:222), and ARTTYSYGSFDY (SEQ ID NO:223) respectively, and/or a light chain with three CDRs comprising the amino acid sequences SSNIGGNS (SEQ ID NO:356), RNN (SEQ ID NO:357), and AAWDDSLNGWV (SEQ ID NO:358) respectively;
v) a heavy chain with three CDRs comprising the amino acid sequences GDSVTSNSAA (SEQ ID NO:224), TYYSSKWYN (SEQ ID NO:225), and ARGWLRLSFDP (SEQ ID NO:226) respectively, and/or a light chain with three CDRs comprising the amino acid sequences SGSIASNY (SEQ ID NO:359), EDN (SEQ ID NO:360), and QSYDPNNHGVV (SEQ ID NO:361) respectively; or
w) a heavy chain with three CDRs comprising the amino acid sequences GFSLTTSGVS (SEQ ID NO:983), IHWDDDK (SEQ ID NO:984), and ASFIMTVYAEYFED (SEQ ID NO:985) respectively, and/or a light chain with three CDRs comprising the amino acid sequences QSVSSN (SEQ ID NO:986), DVS (SEQ ID NO:987), and QQRGAWPLT (SEQ ID NO:988) respectively.

12. The monoclonal antibody of claim 1, wherein the monoclonal antibody comprises:
a) a V_{H} amino acid sequence having SEQ ID NO: 1, and a V_{L} amino acid sequence having SEQ ID NO: 2;
b) a V_{H} amino acid sequence having SEQ ID NO: 3, and a V_{L} amino acid sequence having SEQ ID NO: 4;
c) a V_{H} amino acid sequence having SEQ ID NO: 5, and a V_{L} amino acid sequence having SEQ ID NO: 6;
d) a V_{H} amino acid sequence having SEQ ID NO: 7, and a V_{L} amino acid sequence having SEQ ID NO: 8;
e) a V_{H} amino acid sequence having SEQ ID NO: 9, and a V_{L} amino acid sequence having SEQ ID NO: 10;
f) a V_{H} amino acid sequence having SEQ ID NO: 11, and a V_{L} amino acid sequence having SEQ ID NO: 12;
g) a V_{H} amino acid sequence having SEQ ID NO: 13, and a V_{L} amino acid sequence having SEQ ID NO: 14;
h) a V_{H} amino acid sequence having SEQ ID NO: 19, and a V_{L} amino acid sequence having SEQ ID NO: 20;
i) a V_{H} amino acid sequence having SEQ ID NO: 21, and a V_{L} amino acid sequence having SEQ ID NO: 22;
j) a V_{H} amino acid sequence having SEQ ID NO: 23, and a V_{L} amino acid sequence having SEQ ID NO: 24;
k) a V_{H} amino acid sequence having SEQ ID NO: 25, and a V_{L} amino acid sequence having SEQ ID NO: 26;
1) a V_{H} amino acid sequence having SEQ ID NO: 29, and a V_{L} amino acid sequence having SEQ ID NO: 30;
m) a V_{H} amino acid sequence having SEQ ID NO: 31, and a V_{L} amino acid sequence having SEQ ID NO: 32;
n) a V_{H} amino acid sequence having SEQ ID NO: 33, and a V_{L} amino acid sequence having SEQ ID NO: 34;
o) a V_{H} amino acid sequence having SEQ ID NO: 35, and a V_{L} amino acid sequence having SEQ ID NO: 36;
p) a V_{H} amino acid sequence having SEQ ID NO: 37, and a V_{L} amino acid sequence having SEQ ID NO: 38;
q) a V_{H} amino acid sequence having SEQ ID NO: 39, and a V_{L} amino acid sequence having SEQ ID NO: 40;
r) a V_{H} amino acid sequence having SEQ ID NO: 41, and a V_{L} amino acid sequence having SEQ ID NO: 42;
s) a V_{H} amino acid sequence having SEQ ID NO: 43, and a V_{L} amino acid sequence having SEQ ID NO: 44; or
t) a V_{H} amino acid sequence having SEQ ID NO: 47, and a V_{L} amino acid sequence having SEQ ID NO: 48.

13. The monoclonal antibody of claim 1, wherein the monoclonal antibody comprises:
a) a V_{H} amino acid sequence having SEQ ID NO: 15, and a V_{L} amino acid sequence having SEQ ID NO: 16;
b) a V_{H} amino acid sequence having SEQ ID NO: 17, and a V_{L} amino acid sequence having SEQ ID NO: 18; or
c) a V_{H} amino acid sequence having SEQ ID NO: 27, and a V_{L} amino acid sequence having SEQ ID NO: 28.

14. The monoclonal antibody of claim 1, wherein the monoclonal antibody comprises:
a) a V_{H} amino acid sequence having SEQ ID NO: 49, and a V_{L} amino acid sequence having SEQ ID NO: 50;
b) a V_{H} amino acid sequence having SEQ ID NO: 51, and a V_{L} amino acid sequence having SEQ ID NO: 52;
c) a V_{H} amino acid sequence having SEQ ID NO: 53, and a V_{L} amino acid sequence having SEQ ID NO: 54;
d) a V_{H} amino acid sequence having SEQ ID NO: 55, and a V_{L} amino acid sequence having SEQ ID NO: 56;
e) a V_{H} amino acid sequence having SEQ ID NO: 57, and a V_{L} amino acid sequence having SEQ ID NO: 58;
f) a V_{H} amino acid sequence having SEQ ID NO: 59, and a V_{L} amino acid sequence having SEQ ID NO: 60;
g) a V_{H} amino acid sequence having SEQ ID NO: 61, and a V_{L} amino acid sequence having SEQ ID NO: 62;
h) a V_{H} amino acid sequence having SEQ ID NO: 63, and a V_{L} amino acid sequence having SEQ ID NO: 64;
i) a V_{H} amino acid sequence having SEQ ID NO: 65, and a V_{L} amino acid sequence having SEQ ID NO: 66;
j) a V_{H} amino acid sequence having SEQ ID NO: 67, and a V_{L} amino acid sequence having SEQ ID NO: 68;
k) a V_{H} amino acid sequence having SEQ ID NO: 69, and a V_{L} amino acid sequence having SEQ ID NO: 70;
1) a V_{H} amino acid sequence having SEQ ID NO: 71, and a V_{L} amino acid sequence having SEQ ID NO: 72;
m) a V_{H} amino acid sequence having SEQ ID NO: 73, and a V_{L} amino acid sequence having SEQ ID NO: 74;
n) a V_{H} amino acid sequence having SEQ ID NO: 75, and a V_{L} amino acid sequence having SEQ ID NO: 76;
o) a V_{H} amino acid sequence having SEQ ID NO: 77, and a V_{L} amino acid sequence having SEQ ID NO: 78;
p) a V_{H} amino acid sequence having SEQ ID NO: 79, and a V_{L} amino acid sequence having SEQ ID NO: 80;
q) a V_{H} amino acid sequence having SEQ ID NO: 81, and a V_{L} amino acid sequence having SEQ ID NO: 82;
r) a V_{H} amino acid sequence having SEQ ID NO: 83, and a V_{L} amino acid sequence having SEQ ID NO: 84;
s) a V_{H} amino acid sequence having SEQ ID NO: 85, and a V_{L} amino acid sequence having SEQ ID NO: 86;
t) a V_{H} amino acid sequence having SEQ ID NO: 87, and a V_{L} amino acid sequence having SEQ ID NO: 88;
u) a V_{H} amino acid sequence having SEQ ID NO: 89, and a V_{L} amino acid sequence having SEQ ID NO: 90;
v) a V_{H} amino acid sequence having SEQ ID NO: 91, and a V_{L} amino acid sequence having SEQ ID NO: 92; or
w) a V_{H} amino acid sequence having SEQ ID NO: 981, and a V_{L} amino acid sequence having SEQ ID NO: 982.

15. An isolated scFv antibody, wherein the antibody binds to an epitope in the receptor binding domain (RBD) of the spike protein of a Severe Acute Respiratory Syndrome coronavirus (SARS-CoV2), and neutralizes SARS-CoV2.

16. The antibody of claim 15, wherein the epitope is non-linear.

17. The antibody of claim 16, wherein the epitope comprises a region within amino acids 319-490 of the spike protein (SEQ ID NO: 980).

18. The antibody of claim 16, wherein the epitope comprises a region within amino acids 319-541 of the spike protein (SEQ ID NO: 980).

19. The antibody of claim 15, wherein the antibody inhibits viral and cell membrane fusion.

20. The antibody of claim 15, wherein the antibody competes with the binding of a monoclonal antibody to the spike protein.

21. The antibody of claim 15, wherein the antibody is a fully human antibody.

22. The antibody of claim 15, wherein the antibody blocks the binding of SARS-CoV2 spike protein to angiotensin converting enzyme 2 (ACE2) cell surface receptor.

23. The antibody of claim 15, wherein the antibody comprises:
a) a heavy chain with three CDRs comprising the amino acid sequences GGSIRTHS (SEQ ID NO:93), IHHSGAT (SEQ ID NO:94), and ARGPGILSY (SEQ ID NO:95) respectively, and/or a light chain with three CDRs comprising the amino acid sequences SSNIGSNT (SEQ ID NO:227), SNN (SEQ ID NO:228), and AAWDDSLNVHYV (SEQ ID NO:229) respectively;
b) a heavy chain with three CDRs comprising the amino acid sequences GGSISSYY (SEQ ID NO:96), IYTSGST (SEQ ID NO:97), and ARDVGFGWFDR (SEQ ID NO:98) respectively and/or a light chain with three CDRs comprising the amino acid sequences SGSIASNY (SEQ ID NO:230), EDN (SEQ ID NO:231), and QSFDSASLWV (SEQ ID NO:232) respectively;
c) a heavy chain with three CDRs comprising the amino acid sequences GGSIRTHS (SEQ ID NO:99), IHHSGAT (SEQ ID NO:100), and ARGPGILSY (SEQ ID NO:101) respectively and/or a light chain with three CDRs comprising the amino acid sequences SSNIGSND (SEQ ID NO:233), SNN (SEQ ID NO:234), and ATWDDSLSAGV (SEQ ID NO:235) respectively;
d) a heavy chain with three CDRs comprising the amino acid sequences GDSVSSYSDA (SEQ ID NO:102), TYYRSKWYN (SEQ ID NO:103), and AREIVATTPFRNYYYGMDV (SEQ ID NO: 104) respectively and/or a light chain with three CDRs comprising the amino acid sequences SGSIASNY (SEQ ID NO:236), QDK (SEQ ID NO:237), and QSYDSSSLWV (SEQ ID NO:238) respectively;
e) a heavy chain with three CDRs comprising the amino acid sequences GFTFSHYD (SEQ ID NO:105), IGYDGTNL (SEQ ID NO:106), and ARAANYYDSSGYGRADAFDI (SEQ ID NO:107) respectively and/or a light chain with three CDRs comprising the amino acid sequences TGSIAGNY (SEQ ID NO:239), DDN (SEQ ID NO:240), and QSYDSGNRGV (SEQ ID NO:241) respectively;
f) a heavy chain with three CDRs comprising the amino acid sequences GFTFSDFP (SEQ ID NO: 108), ISYDGNIK (SEQ ID NO:109), and A ARGGSSFDI (SEQ ID NO:110) respectively and/or a light chain with three CDRs comprising the amino acid sequences TSNIGNNA (SEQ ID NO:242), YNE (SEQ ID NO:243), and AAWDDSLSGHVV (SEQ ID NO:244) respectively;
g) a heavy chain with three CDRs comprising the amino acid sequences GFSLSTTGVG (SEQ ID NO:111), IYWNDDK (SEQ ID NO:112), and ARISGSGYFYPFDI (SEQ ID NO:113) respectively and/or a light chain with three CDRs comprising the amino acid sequences SGSIASNY (SEQ ID NO:245), EDN (SEQ ID NO:246), and QSYDSSSLWV (SEQ ID NO:247) respectively;
h) a heavy chain with three CDRs comprising the amino acid sequences GYTFSDYY (SEQ ID NO:120), IDPNSGGT (SEQ ID NO:121), and ARDRGRGGQAGAFDY (SEQ ID NO:978) respectively and/or a light chain with three CDRs comprising the amino acid sequences KIGSKS (SEQ ID NO:254), DDS (SEQ ID NO:255), and HVWDSSSDQNV (SEQ ID NO:256) respectively;
i) a heavy chain with three CDRs comprising the amino acid sequences GFTFSSYA (SEQ ID NO:122), ISYGGSNK (SEQ ID NO:123), and AKVRGSGWYWGSAFDI (SEQ ID NO:124) respectively and/or a light chain with three CDRs comprising the amino acid sequences SLRAYF (SEQ ID NO:257), GQD (SEQ ID NO:258), and NSRDSGENHLI (SEQ ID NO:259) respectively;
j) a heavy chain with three CDRs comprising the amino acid sequences GYSFTGSH (SEQ ID NO:125), INPDSGVI (SEQ ID NO:126), and ARDKAIGYVWALDY (SEQ ID NO:127) respectively and/or a light chain with three CDRs comprising the amino acid sequences SSDVGTYNR (SEQ ID NO:260), EVS (SEQ ID NO:261), and SSYTRTFTYV (SEQ ID NO:262) respectively;
k) a heavy chain with three CDRs comprising the amino acid sequences GVSLDTIGMR (SEQ ID NO:128), IDWDDDK (SEQ ID NO: 129), and ARSGLLYDLDV (SEQ ID NO:130) respectively and/or a light chain with three CDRs comprising the amino acid sequences DSDIGANF (SEQ ID NO:263), RNT (SEQ ID NO:264), and QSYDSSLSAYV (SEQ ID NO:265) respectively;
l) a heavy chain with three CDRs comprising the amino acid sequences GYSFTSYW (SEQ ID NO:134), IYPGDSDT (SEQ ID NO:135), and ARGWQWHDY (SEQ ID NO:136) respectively and/or a light chain with three CDRs comprising the amino acid sequences SLRSYY (SEQ ID NO:269), DKD (SEQ ID NO:270), and NSRDRSDNHVV (SEQ ID NO:271) respectively;
m) a heavy chain with three CDRs comprising the amino acid sequences GDSVSSRSSA (SEQ ID NO:137), TYYRSNWNY (SEQ ID NO:138), and VRNMRPDFDL (SEQ ID NO: 139) respectively and/or a light chain with three CDRs comprising the amino acid sequences QSVSNN (SEQ ID NO:272), DAT (SEQ ID NO:273), and QQYDNLPV (SEQ ID NO:274) respectively;
n) a heavy chain with three CDRs comprising the amino acid sequences GYTFTTSG (SEQ ID NO: 140), ISAYNGNT (SEQ ID NO:141), and ARDFHLYYGMDV (SEQ ID NO:142) respectively and/or a light chain with three CDRs comprising the amino acid sequences SSDVGAYNY (SEQ ID NO:275), DVT (SEQ ID NO:276), and AVWDDGLNGRVV (SEQ ID NO:277) respectively;
o) a heavy chain with three CDRs comprising the amino acid sequences GGTFSSYA (SEQ ID NO:143), INPNSGGT (SEQ ID NO:144), and ARGSGGYYLG (SEQ ID NO:145) respectively and/or a light chain with three CDRs comprising the amino acid sequences SNNVGNQG (SEQ ID NO:278), MNN (SEQ ID NO:279), and SAWDSSLSRWV (SEQ ID NO:280) respectively;
p) a heavy chain with three CDRs comprising the amino acid sequences GGTFSSYT (SEQ ID NO:146), IIPILGTP (SEQ ID NO:147), and AVGSGWYSGFDY (SEQ ID NO:148) respectively and/or a light chain with three CDRs comprising the amino acid sequences SGSIASNY (SEQ ID NO:281), EDS (SEQ ID NO:282), and QSFHNSNPVI (SEQ ID NO:283) respectively;
q) a heavy chain with three CDRs comprising the amino acid sequences GFTFSSYW (SEQ ID NO:149), IKQDGSEK (SEQ ID NO:150), and ARGFYYYGAFDI (SEQ ID NO:151) respectively and/or a light chain with three CDRs comprising the amino acid sequences SGSIASNY (SEQ ID NO:284), EDN (SEQ ID NO:285), and QSYDSSNHWV (SEQ ID NO:286) respectively;
r) a heavy chain with three CDRs comprising the amino acid sequences GFTFDDYA (SEQ ID NO:152), IDWNSGVI (SEQ ID NO:153), and AKDAYSYGFLGAFDI (SEQ ID NO:154) respectively and/or a light chain with three CDRs comprising the amino acid sequences NIGSKS (SEQ ID NO:287), EDR (SEQ ID NO:288), and QVWDGDSDHYV (SEQ ID NO:289) respectively;
s) a heavy chain with three CDRs comprising the amino acid sequences GFTFDDYA (SEQ ID NO:155), IDWNSGVI (SEQ ID NO:156), and ARDILPSNFDGKKIIVFQPPAKRDLDNYYGMDV (SEQ ID NO:157) respectively and/or a light chain with three CDRs comprising the amino acid sequences SSDVGGYNL (SEQ ID NO:290), EGS (SEQ ID NO:291), and SSYTITDVVV (SEQ ID NO:292) respectively; or
t) a heavy chain with three CDRs comprising the amino acid sequences GYSFTSNW (SEQ ID NO:158), IFPGDSDT (SEQ ID NO:159), and ARESYNAYGS (SEQ ID NO:160) respectively and/or a light chain with three CDRs comprising the amino acid sequences SSNIGSNP (SEQ ID NO:293), SNN (SEQ ID NO:294), and AAWDDSLSGVV (SEQ ID NO:295) respectively.

24. The antibody of claim 15, wherein the antibody comprises:
a) a heavy chain with three CDRs comprising the amino acid sequences GFTFTTYG (SEQ ID NO:114), ISYDGSIK (SEQ ID NO:115), and ARVGDSSSYYGIDA (SEQ ID NO:116) respectively, and/or a light chain with three CDRs comprising the amino acid sequences SSNIGSNS (SEQ ID NO:248), SNN (SEQ ID NO:249), and AAWDDSLTGYV (SEQ ID NO:250) respectively;
b) a heavy chain with three CDRs comprising the amino acid sequences GFTFSSHA (SEQ ID NO:117), ISYDGSYT (SEQ ID NO:118), and ARDWVNFGMDV (SEQ ID NO:119) respectively, and/or a light chain with three CDRs comprising the amino acid sequences SSDVGGYNY (SEQ ID NO:251), EVS (SEQ ID NO:252), and AAWDDSLSGPV (SEQ ID NO:253) respectively; or
c) a heavy chain with three CDRs comprising the amino acid sequences GFTFSDYP (SEQ ID NO:131), TSYDGRIK (SEQ ID NO:132), and ARDPGWLRSVGMDV (SEQ ID NO:133) respectively, and/or a light chain with three CDRs comprising the amino acid sequences SGSIARNY (SEQ ID NO:266), ADR (SEQ ID NO:267), and QSYDSSNQAAV (SEQ ID NO:268) respectively.

25. The antibody of claim 15, wherein the antibody comprises:
a) a heavy chain with three CDRs comprising the amino acid sequences GYTFTSYG (SEQ ID NO:161), ISAYNGNT (SEQ ID NO:162), and ARGFPQLGSDY (SEQ ID NO: 163) respectively, and/or a light chain with three CDRs comprising the amino acid sequences SGSIASNY (SEQ ID NO:296), EDN (SEQ ID NO:297), and QAWDSNSYV (SEQ ID NO:298) respectively;
b) a heavy chain with three CDRs comprising the amino acid sequences GGTFSSYA (SEQ ID NO:164), ISGYNGNT (SEQ ID NO:165), and ARQMKDSGNYWEYYYYGMDV (SEQ ID NO:166) respectively, and/or a light chain with three CDRs comprising the amino acid sequences NIGSES (SEQ ID NO:299), EDR (SEQ ID NO:300), and QVWNPSGSLQYV (SEQ ID NO:301) respectively;
c) a heavy chain with three CDRs comprising the amino acid sequences GYTFTSYG (SEQ ID NO:167), ISTYNGNT (SEQ ID NO:168), and ARDVFGHFDY (SEQ ID NO:169) respectively, and/or a light chain with three CDRs comprising the amino acid sequences SGNIATNY (SEQ ID NO:302), EDN (SEQ ID NO:303), and KSYDDGNHV (SEQ ID NO:304) respectively;
d) a heavy chain with three CDRs comprising the amino acid sequences GFSLTTTGVS (SEQ ID NO:170), IHWDDDK (SEQ ID NO:171), and ASFIMTVYAEYFED (SEQ ID NO: 172) respectively, and/or a light chain with three CDRs comprising the amino acid sequences QSVSSN (SEQ ID NO:305), DVS (SEQ ID NO:306), and QQRGVWPLT (SEQ ID NO:307) respectively;
e) a heavy chain with three CDRs comprising the amino acid sequences GFSLSTSAMC (SEQ ID NO:173), IDWDNDR (SEQ ID NO:174), and AHSPYDSIWGSFRPSVYYFDY (SEQ ID NO:175) respectively, and/or a light chain with three CDRs comprising the amino acid sequences SGSIVSSY (SEQ ID NO:308), EHN (SEQ ID NO:309), and QSYDSQNGV (SEQ ID NO:310) respectively;
f) a heavy chain with three CDRs comprising the amino acid sequences GFTFSDYY (SEQ ID NO:176), ISSSSSDT (SEQ ID NO:177), and AMPTREPAY (SEQ ID NO:178) respectively, and/or a light chain with three CDRs comprising the amino acid sequences SSDLGTYNY (SEQ ID NO:311), DVF (SEQ ID NO:312), and SSYTSSSTYV (SEQ ID NO:313) respectively;
g) a heavy chain with three CDRs comprising the amino acid sequences GFAFSDFP (SEQ ID NO: 179), ISYDGSLK (SEQ ID NO:180), and AREGVSNSRPFDH (SEQ ID NO:181) respectively, and/or a light chain with three CDRs comprising the amino acid sequences SIGTKS (SEQ ID NO:314), DDS (SEQ ID NO:315), and QVWESDDDDLV (SEQ ID NO:316) respectively;
h) a heavy chain with three CDRs comprising the amino acid sequences GFTFSSYA (SEQ ID NO:182), ISSNGGST (SEQ ID NO:183), and TRDLWSGSADSFDI (SEQ ID NO:184) respectively, and/or a light chain with three CDRs comprising the amino acid sequences SLRRYY (SEQ ID NO:317), GKN (SEQ ID NO:318), and NSRDISDNQWQWI (SEQ ID NO:319) respectively;
i) a heavy chain with three CDRs comprising the amino acid sequences GFPFNAYY (SEQ ID NO:185), INQDGSEK (SEQ ID NO:186), and ARLYWWGMDV (SEQ ID NO:187) respectively, and/or a light chain with three CDRs comprising the amino acid sequences SSDVGGYKY (SEQ ID NO:320), DVN (SEQ ID NO:321), and SSYTGRMNLYV (SEQ ID NO:322) respectively;
j) a heavy chain with three CDRs comprising the amino acid sequences GFTFDDYA (SEQ ID NO:188), IDWNSGVI (SEQ ID NO:189), and AKDAYSYGFLGAFDI (SEQ ID NO:190) respectively, and/or a light chain with three CDRs comprising the amino acid sequences NIRTKG (SEQ ID NO:323), YAS (SEQ ID NO:324), and QVWDSSSDLVV (SEQ ID NO:325) respectively;
k) a heavy chain with three CDRs comprising the amino acid sequences GFTFDDYA (SEQ ID NO:191), ISWNSGSI (SEQ ID NO:192), and ARDWWGSIDH (SEQ ID NO:193) respectively, and/or a light chain with three CDRs comprising the amino acid sequences SSDVGGYDY (SEQ ID NO:326), DVS (SEQ ID NO:327), and SSYTSSSPVV (SEQ ID NO:328) respectively;
l) a heavy chain with three CDRs comprising the amino acid sequences GGSISSSNW (SEQ ID NO:194), IYHSGST (SEQ ID NO:195), and ARRGGTYHRGAFDI (SEQ ID NO:196) respectively, and/or a light chain with three CDRs comprising the amino acid sequences SRDVGSYDL (SEQ ID NO:329), EGS (SEQ ID NO:330), and SSYTSSNSLV (SEQ ID NO:331) respectively;
m) a heavy chain with three CDRs comprising the amino acid sequences GASISNSF (SEQ ID NO:197), TSYSGNS (SEQ ID NO:198), and ARREWIKGHFDY (SEQ ID NO:199) respectively, and/or a light chain with three CDRs comprising the amino acid sequences GGSIASNY (SEQ ID NO:332), EDN (SEQ ID NO:333), and QSYDSSNPVV (SEQ ID NO:334) respectively;
n) a heavy chain with three CDRs comprising the amino acid sequences GGSFTTHS (SEQ ID NO:200), ILPGGAT (SEQ ID NO:201), and ARGPGILSY (SEQ ID NO:202) respectively, and/or a light chain with three CDRs comprising the amino acid sequences SSIGSND (SEQ ID NO:335), SNN (SEQ ID NO:336), and AWDDSLSAVV (SEQ ID NO:337) respectively;
o) a heavy chain with three CDRs comprising the amino acid sequences GGSFRTHS (SEQ ID NO:203), IHHSGAT (SEQ ID NO:204), and ARGPGILSY (SEQ ID NO:205) respectively, and/or a light chain with three CDRs comprising the amino acid sequences SSNIGSNT (SEQ ID NO:338), INN (SEQ ID NO:339), and AEWYDSLNVHYV (SEQ ID NO:340) respectively;
p) a heavy chain with three CDRs comprising the amino acid sequences GGSIRTHS (SEQ ID NO:206), IHHSGAT (SEQ ID NO:207), and ARGPGILSY (SEQ ID NO:208) respectively, and/or a light chain with three CDRs comprising the amino acid sequences SSNIGSNT (SEQ ID NO:341), INN (SEQ ID NO:342), and AECYDSLNDHYV (SEQ ID NO:343) respectively;
q) a heavy chain with three CDRs comprising the amino acid sequences GGSIRTHS (SEQ ID NO:209), IHHSGAT (SEQ ID NO:210), and GRGPGILSY (SEQ ID NO:211) respectively, and/or a light chain with three CDRs comprising the amino acid sequences SSNIGSNT (SEQ ID NO:344), SNN (SEQ ID NO:345), and AAWDDSLNVHYV (SEQ ID NO:346) respectively;
r) a heavy chain with three CDRs comprising the amino acid sequences GYSFTSYW (SEQ ID NO:212), IYPGDSDT (SEQ ID NO:213), and ARQGDGGGYDY (SEQ ID NO:214) respectively, and/or a light chain with three CDRs comprising the amino acid sequences SSNIGSNP (SEQ ID NO:347), NNN (SEQ ID NO:348), and AAWDDSLNGL (SEQ ID NO:349) respectively;
s) a heavy chain with three CDRs comprising the amino acid sequences RYSFSNYW (SEQ ID NO:215), IYPYDSDT (SEQ ID NO:216), and ARQGSSQSFDI (SEQ ID NO:217) respectively, and/or a light chain with three CDRs comprising the amino acid sequences SLRSYY (SEQ ID NO:350), QDS (SEQ ID NO:351), and QAWDSNSYV (SEQ ID NO:352) respectively;
t) a heavy chain with three CDRs comprising the amino acid sequences GYSFTSYW (SEQ ID NO:218), IYPGDSDT (SEQ ID NO:219), and ARRRGSAAAFDT (SEQ ID NO:220) respectively, and/or a light chain with three CDRs comprising the amino acid sequences SSNIGSNP (SEQ ID NO:353), DNN (SEQ ID NO:354), and EAWDDSLSGPV (SEQ ID NO:355) respectively;
u) a heavy chain with three CDRs comprising the amino acid sequences GYSFTSYW (SEQ ID NO:221), IYPGDSDT (SEQ ID NO:222), and ARTTYSYGSFDY (SEQ ID NO:223) respectively, and/or a light chain with three CDRs comprising the amino acid sequences SSNIGGNS (SEQ ID NO:356), RNN (SEQ ID NO:357), and AAWDDSLNGWV (SEQ ID NO:358) respectively;
v) a heavy chain with three CDRs comprising the amino acid sequences GDSVTSNSAA (SEQ ID NO:224), TYYSSKWYN (SEQ ID NO:225), and ARGWLRLSFDP (SEQ ID NO:226) respectively, and/or a light chain with three CDRs comprising the amino acid sequences SGSIASNY (SEQ ID NO:359), EDN (SEQ ID NO:360), and QSYDPNNHGVV (SEQ ID NO:361) respectively; or
w) a heavy chain with three CDRs comprising the amino acid sequences GFSLTTSGVS (SEQ ID NO:983), IHWDDDK (SEQ ID NO:984), and ASFIMTVYAEYFED (SEQ ID NO:985) respectively, and/or a light chain with three CDRs comprising the amino acid sequences QSVSSN (SEQ ID NO:986), DVS (SEQ ID NO:987), and QQRGAWPLT (SEQ ID NO:988) respectively.

26. The antibody of claim 15, wherein the antibody comprises:
a) a V_{H} amino acid sequence having SEQ ID NO: 1, and a V_{L} amino acid sequence having SEQ ID NO: 2;
b) a V_{H} amino acid sequence having SEQ ID NO: 3, and a V_{L} amino acid sequence having SEQ ID NO: 4;
c) a V_{H} amino acid sequence having SEQ ID NO: 5, and a V_{L} amino acid sequence having SEQ ID NO: 6;
d) a V_{H} amino acid sequence having SEQ ID NO: 7, and a V_{L} amino acid sequence having SEQ ID NO: 8;
e) a V_{H} amino acid sequence having SEQ ID NO: 9, and a V_{L} amino acid sequence having SEQ ID NO: 10;
f) a V_{H} amino acid sequence having SEQ ID NO: 11, and a V_{L} amino acid sequence having SEQ ID NO: 12;
g) a V_{H} amino acid sequence having SEQ ID NO: 13, and a V_{L} amino acid sequence having SEQ ID NO: 14;
h) a V_{H} amino acid sequence having SEQ ID NO: 19, and a V_{L} amino acid sequence having SEQ ID NO: 20;
i) a V_{H} amino acid sequence having SEQ ID NO: 21, and a V_{L} amino acid sequence having SEQ ID NO: 22;
j) a V_{H} amino acid sequence having SEQ ID NO: 23, and a V_{L} amino acid sequence having SEQ ID NO: 24;
k) a V_{H} amino acid sequence having SEQ ID NO: 25, and a V_{L} amino acid sequence having SEQ ID NO: 26;
l) a V_{H} amino acid sequence having SEQ ID NO: 29, and a V_{L} amino acid sequence having SEQ ID NO: 30;
m) a V_{H} amino acid sequence having SEQ ID NO: 31, and a V_{L} amino acid sequence having SEQ ID NO: 32;
n) a V_{H} amino acid sequence having SEQ ID NO: 33, and a V_{L} amino acid sequence having SEQ ID NO: 34;
o) a V_{H} amino acid sequence having SEQ ID NO: 35, and a V_{L} amino acid sequence having SEQ ID NO: 36;
p) a V_{H} amino acid sequence having SEQ ID NO: 37, and a V_{L} amino acid sequence having SEQ ID NO: 38;
q) a V_{H} amino acid sequence having SEQ ID NO: 39, and a V_{L} amino acid sequence having SEQ ID NO: 40;
r) a V_{H} amino acid sequence having SEQ ID NO: 41, and a V_{L} amino acid sequence having SEQ ID NO: 42;
s) a V_{H} amino acid sequence having SEQ ID NO: 43, and a V_{L} amino acid sequence having SEQ ID NO: 44; or
t) a V_{H} amino acid sequence having SEQ ID NO: 47, and a V_{L} amino acid sequence having SEQ ID NO: 48.

27. The antibody of claim 15, wherein the antibody comprises:
a) a V_{H} amino acid sequence having SEQ ID NO: 15, and a V_{L} amino acid sequence having SEQ ID NO: 16;
b) a V_{H} amino acid sequence having SEQ ID NO: 17, and a V_{L} amino acid sequence having SEQ ID NO: 18; or
c) a V_{H} amino acid sequence having SEQ ID NO: 27, and a V_{L} amino acid sequence having SEQ ID NO: 28.

28. The antibody of claim 15, wherein the antibody comprises:
a) a V_{H} amino acid sequence having SEQ ID NO: 49, and a V_{L} amino acid sequence having SEQ ID NO: 50;
b) a V_{H} amino acid sequence having SEQ ID NO: 51, and a V_{L} amino acid sequence having SEQ ID NO: 52;
c) a V_{H} amino acid sequence having SEQ ID NO: 53, and a V_{L} amino acid sequence having SEQ ID NO: 54;
d) a V_{H} amino acid sequence having SEQ ID NO: 55, and a V_{L} amino acid sequence having SEQ ID NO: 56;
e) a V_{H} amino acid sequence having SEQ ID NO: 57, and a V_{L} amino acid sequence having SEQ ID NO: 58;
f) a V_{H} amino acid sequence having SEQ ID NO: 59, and a V_{L} amino acid sequence having SEQ ID NO: 60;
g) a V_{H} amino acid sequence having SEQ ID NO: 61, and a V_{L} amino acid sequence having SEQ ID NO: 62;
h) a V_{H} amino acid sequence having SEQ ID NO: 63, and a V_{L} amino acid sequence having SEQ ID NO: 64;
i) a V_{H} amino acid sequence having SEQ ID NO: 65, and a V_{L} amino acid sequence having SEQ ID NO: 66;
j) a V_{H} amino acid sequence having SEQ ID NO: 67, and a V_{L} amino acid sequence having SEQ ID NO: 68;
k) a V_{H} amino acid sequence having SEQ ID NO: 69, and a V_{L} amino acid sequence having SEQ ID NO: 70;
1) a V_{H} amino acid sequence having SEQ ID NO: 71, and a V_{L} amino acid sequence having SEQ ID NO: 72;
m) a V_{H} amino acid sequence having SEQ ID NO: 73, and a V_{L} amino acid sequence having SEQ ID NO: 74;
n) a V_{H} amino acid sequence having SEQ ID NO: 75, and a V_{L} amino acid sequence having SEQ ID NO: 76;
o) a V_{H} amino acid sequence having SEQ ID NO: 77, and a V_{L} amino acid sequence having SEQ ID NO: 78;
p) a V_{H} amino acid sequence having SEQ ID NO: 79, and a V_{L} amino acid sequence having SEQ ID NO: 80;
q) a V_{H} amino acid sequence having SEQ ID NO: 81, and a V_{L} amino acid sequence having SEQ ID NO: 82;
r) a V_{H} amino acid sequence having SEQ ID NO: 83, and a V_{L} amino acid sequence having SEQ ID NO: 84;
s) a V_{H} amino acid sequence having SEQ ID NO: 85, and a V_{L} amino acid sequence having SEQ ID NO: 86;
t) a V_{H} amino acid sequence having SEQ ID NO: 87, and a V_{L} amino acid sequence having SEQ ID NO: 88;
u) a V_{H} amino acid sequence having SEQ ID NO: 89, and a V_{L} amino acid sequence having SEQ ID NO: 90; or
v) a V_{H} amino acid sequence having SEQ ID NO: 91, and a V_{L} amino acid sequence having SEQ ID NO: 92.

29. The monoclonal antibody of claim 1, wherein the monoclonal antibody comprises:
a) a heavy chain with three CDRs comprising the amino acid sequences GFSLSTSGVG (SEQ ID NO:754), IYWDDDK (SEQ ID NO:755), and ARISGSGYFYPFDI (SEQ ID NO:756) respectively, and/or a light chain with three CDRs comprising the amino acid sequences SGSIASNY (SEQ ID NO:802), EDN (SEQ ID NO:803), and QSYDSSNLWV (SEQ ID NO:804) respectively;
b) a heavy chain with three CDRs comprising the amino acid sequences GDSVSSNSAA (SEQ ID NO:757), TYYRSRWYN (SEQ ID NO:758), and AREIRGFDY (SEQ ID NO:759) respectively, and/or a light chain with three CDRs comprising the amino acid sequences SSDVGAYNF (SEQ ID NO:805), DFN (SEQ ID NO:806), and SSYAGSNNFDVV (SEQ ID NO:807) respectively;
c) a heavy chain with three CDRs comprising the amino acid sequences GFTFGDYA (SEQ ID NO:760), IRSKAYGGTT (SEQ ID NO:761), and TTADDDMDV (SEQ ID NO:762) respectively, and/or a light chain with three CDRs comprising the amino acid sequences SGTIASNY (SEQ ID NO:808), EDN (SEQ ID NO:809), and QSYDTSNHYV (SEQ ID NO:810) respectively;
d) a heavy chain with three CDRs comprising the amino acid sequences GFTFSNYG (SEQ ID NO:763), IWERGSKK (SEQ ID NO:764), and AREGISMTGAEYFQH (SEQ ID NO:765) respectively, and/or a light chain with three CDRs comprising the amino acid sequences SSNIGAGYD (SEQ ID NO:811), GTN (SEQ ID NO:812), and QSYDNSLTDPYV (SEQ ID NO:813) respectively;
e) a heavy chain with three CDRs comprising the amino acid sequences GFTFDDYA (SEQ ID NO:766), IDWNSGVI (SEQ ID NO:767), and AKDIGPGGSGSYYAFDI (SEQ ID NO:768) respectively, and/or a light chain with three CDRs comprising the amino acid sequences SSDVGGSKY (SEQ ID NO:814), DVT (SEQ ID NO:815), and AAWDDSLNGVV (SEQ ID NO:816) respectively;
f) a heavy chain with three CDRs comprising the amino acid sequences GFSFSRYG (SEQ ID NO:769), IRHDGSKK (SEQ ID NO:770), and AKDGRLEAALDD (SEQ ID NO:771) respectively, and/or a light chain with three CDRs comprising the amino acid sequences SGSIANNF (SEQ ID NO:817), EDN (SEQ ID NO:818), and QSYDSSNLV (SEQ ID NO:819) respectively;
g) a heavy chain with three CDRs comprising the amino acid sequences GYSFTSYW (SEQ ID NO:772), IYPGDSDT (SEQ ID NO:773), and ARRGDLDAFDI (SEQ ID NO:774) respectively, and/or a light chain with three CDRs comprising the amino acid sequences SANIGSNA (SEQ ID NO:820), GNT (SEQ ID NO:821), and AAWDDSLNGYV (SEQ ID NO:822) respectively;
h) a heavy chain with three CDRs comprising the amino acid sequences GYRLSDYY (SEQ ID NO:775), IKQDGSEK (SEQ ID NO:776), and ARVRGWSRGYFDY (SEQ ID NO:777) respectively, and/or a light chain with three CDRs comprising the amino acid sequences SGSIASNY (SEQ ID NO:823), EDN (SEQ ID NO:824), and QSYDSSNHWV (SEQ ID NO:825) respectively;
i) a heavy chain with three CDRs comprising the amino acid sequences GFTFDDYA (SEQ ID NO:778), ISWNSGSI (SEQ ID NO:779), and ARDWWGSIDH (SEQ ID NO:780) respectively, and/or a light chain with three CDRs comprising the amino acid sequences SSDVGGYDY (SEQ ID NO:826), DVS (SEQ ID NO:827), and SSYTSSSPVV (SEQ ID NO:828) respectively;
j) a heavy chain with three CDRs comprising the amino acid sequences GFTFSHYD (SEQ ID NO:781), IGYDGTNL (SEQ ID NO:782), and ARAANYYDSSGYGRADAF (SEQ ID NO:783) respectively, and/or a light chain with three CDRs comprising the amino acid sequences TGSIAGNY (SEQ ID NO:829), DDN (SEQ ID NO:830), and QSYDSGNRGV (SEQ ID NO:831) respectively;
k) a heavy chain with three CDRs comprising the amino acid sequences GGTFSTYG (SEQ ID NO:784), IIPSLGIP (SEQ ID NO:785), and ARENIDLATNDF (SEQ ID NO:786) respectively, and/or a light chain with three CDRs comprising the amino acid sequences SRDIGAYGY (SEQ ID NO:832), EVR (SEQ ID NO:833), and SSYTSSSTLDVV (SEQ ID NO:834) respectively;
l) a heavy chain with three CDRs comprising the amino acid sequences GGTFSSSG (SEQ ID NO:787), IIPMLGTP (SEQ ID NO:788), and ARDGGNYDY (SEQ ID NO:789) respectively, and/or a light chain with three CDRs comprising the amino acid sequences SSNIGRNA (SEQ ID NO:835), SNN (SEQ ID NO:836), and SAWDTSLSTWV (SEQ ID NO:837) respectively;
m) a heavy chain with three CDRs comprising the amino acid sequences GFTFSSYW (SEQ ID NO:790), IKQDGSEK (SEQ ID NO:791), and ARGFYYYGAFDI (SEQ ID NO:792) respectively, and/or a light chain with three CDRs comprising the amino acid sequences SGSIASNY (SEQ ID NO:838), EDN (SEQ ID NO:839), and QSYDSSNHWV (SEQ ID NO:840) respectively;
n) a heavy chain with three CDRs comprising the amino acid sequences GFTFDDYA (SEQ ID NO:793), IDWNSGVI (SEQ ID NO:794), and AKDAYSYGFLGAFDI (SEQ ID NO:795) respectively, and/or a light chain with three CDRs comprising the amino acid sequences NIRTKG (SEQ ID NO:841), YAS (SEQ ID NO:842), and QVWDSSSDLVV (SEQ ID NO:843) respectively;
o) a heavy chain with three CDRs comprising the amino acid sequences GYSFTGSH (SEQ ID NO:796), INPDSGVI (SEQ ID NO:797), and ARDKAIGYVWALDY (SEQ ID NO:798) respectively, and/or a light chain with three CDRs comprising the amino acid sequences SSDVGTYNR (SEQ ID NO:844), EVS (SEQ ID NO:845), and SSYTRTFTYV (SEQ ID NO:846) respectively; or
p) a heavy chain with three CDRs comprising the amino acid sequences GASISNSF (SEQ ID NO:799), TSYSGNS (SEQ ID NO:800), and ARREWIKGHFDY (SEQ ID NO:801) respectively, and/or a light chain with three CDRs comprising the amino acid sequences GGSIASNY (SEQ ID NO:847), EDN (SEQ ID NO:848), and QSYDSSNPVV (SEQ ID NO:849) respectively.

30. The monoclonal antibody of claim 1, wherein the monoclonal antibody comprises:
a) a V_{H} amino acid sequence having SEQ ID NO: 722, and a V_{L} amino acid sequence having SEQ ID NO: 723;
b) a V_{H} amino acid sequence having SEQ ID NO: 724, and a V_{L} amino acid sequence having SEQ ID NO: 725;
c) a V_{H} amino acid sequence having SEQ ID NO: 726, and a V_{L} amino acid sequence having SEQ ID NO: 727;
d) a V_{H} amino acid sequence having SEQ ID NO: 728, and a V_{L} amino acid sequence having SEQ ID NO: 729;
e) a V_{H} amino acid sequence having SEQ ID NO: 730, and a V_{L} amino acid sequence having SEQ ID NO: 731;
f) a V_{H} amino acid sequence having SEQ ID NO: 732, and a V_{L} amino acid sequence having SEQ ID NO: 733;
g) a V_{H} amino acid sequence having SEQ ID NO: 734, and a V_{L} amino acid sequence having SEQ ID NO: 735;
h) a V_{H} amino acid sequence having SEQ ID NO: 736, and a V_{L} amino acid sequence having SEQ ID NO: 737;
i) a V_{H} amino acid sequence having SEQ ID NO: 738, and a V_{L} amino acid sequence having SEQ ID NO: 739;
j) a V_{H} amino acid sequence having SEQ ID NO: 740, and a V_{L} amino acid sequence having SEQ ID NO: 741;
k) a V_{H} amino acid sequence having SEQ ID NO: 742, and a V_{L} amino acid sequence having SEQ ID NO: 743;
l) a V_{H} amino acid sequence having SEQ ID NO: 744, and a V_{L} amino acid sequence having SEQ ID NO: 745;
m) a V_{H} amino acid sequence having SEQ ID NO: 746, and a V_{L} amino acid sequence having SEQ ID NO: 747;
n) a V_{H} amino acid sequence having SEQ ID NO: 748, and a V_{L} amino acid sequence having SEQ ID NO: 749;
o) a V_{H} amino acid sequence having SEQ ID NO: 750, and a V_{L} amino acid sequence having SEQ ID NO: 751; or
p) a V_{H} amino acid sequence having SEQ ID NO: 752, and a V_{L} amino acid sequence having SEQ ID NO: 753.

31. The antibody of claim 15, wherein the antibody comprises:
a) a heavy chain with three CDRs comprising the amino acid sequences GFSLSTSGVG (SEQ ID NO:754), IYWDDDK (SEQ ID NO:755), and ARISGSGYFYPFDI (SEQ ID NO:756) respectively, and/or a light chain with three CDRs comprising the amino acid sequences SGSIASNY (SEQ ID NO:802), EDN (SEQ ID NO:803), and QSYDSSNLWV (SEQ ID NO:804) respectively;
b) a heavy chain with three CDRs comprising the amino acid sequences GDSVSSNSAA (SEQ ID NO:757), TYYRSRWYN (SEQ ID NO:758), and AREIRGFDY (SEQ ID NO:759) respectively, and/or a light chain with three CDRs comprising the amino acid sequences SSDVGAYNF (SEQ ID NO:805), DFN (SEQ ID NO:806), and SSYAGSNNFDVV (SEQ ID NO:807) respectively;
c) a heavy chain with three CDRs comprising the amino acid sequences GFTFGDYA (SEQ ID NO:760), IRSKAYGGTT (SEQ ID NO:761), and TTADDDMDV (SEQ ID NO:762) respectively, and/or a light chain with three CDRs comprising the amino acid sequences SGTIASNY (SEQ ID NO:808), EDN (SEQ ID NO:809), and QSYDTSNHYV (SEQ ID NO:810) respectively;
d) a heavy chain with three CDRs comprising the amino acid sequences GFTFSNYG (SEQ ID NO:763), IWERGSKK (SEQ ID NO:764), and AREGISMTGAEYFQH (SEQ ID NO:765) respectively, and/or a light chain with three CDRs comprising the amino acid sequences SSNIGAGYD (SEQ ID NO:811), GTN (SEQ ID NO:812), and QSYDNSLTDPYV (SEQ ID NO:813) respectively;
e) a heavy chain with three CDRs comprising the amino acid sequences GFTFDDYA (SEQ ID NO:766), IDWNSGVI (SEQ ID NO:767), and AKDIGPGGSGSYYAFDI (SEQ ID NO:768) respectively, and/or a light chain with three CDRs comprising the amino acid sequences SSDVGGSKY (SEQ ID NO:814), DVT (SEQ ID NO:815), and AAWDDSLNGVV (SEQ ID NO:816) respectively;
f) a heavy chain with three CDRs comprising the amino acid sequences GFSFSRYG (SEQ ID NO:769), IRHDGSKK (SEQ ID NO:770), and AKDGRLEAALDD (SEQ ID NO:771) respectively, and/or a light chain with three CDRs comprising the amino acid sequences SGSIANNF (SEQ ID NO:817), EDN (SEQ ID NO:818), and QSYDSSNLV (SEQ ID NO:819) respectively;
g) a heavy chain with three CDRs comprising the amino acid sequences GYSFTSYW (SEQ ID NO:772), IYPGDSDT (SEQ ID NO:773), and ARRGDLDAFDI (SEQ ID NO:774) respectively, and/or a light chain with three CDRs comprising the amino acid sequences SANIGSNA (SEQ ID NO:820), GNT (SEQ ID NO:821), and AAWDDSLNGYV (SEQ ID NO:822) respectively;
h) a heavy chain with three CDRs comprising the amino acid sequences GYRLSDYY (SEQ ID NO:775), IKQDGSEK (SEQ ID NO:776), and ARVRGWSRGYFDY (SEQ ID NO:777) respectively, and/or a light chain with three CDRs comprising the amino acid sequences SGSIASNY (SEQ ID NO:823), EDN (SEQ ID NO:824), and QSYDSSNHWV (SEQ ID NO:825) respectively;
i) a heavy chain with three CDRs comprising the amino acid sequences GFTFDDYA (SEQ ID NO:778), ISWNSGSI (SEQ ID NO:779), and ARDWWGSIDH (SEQ ID NO:780) respectively, and/or a light chain with three CDRs comprising the amino acid sequences SSDVGGYDY (SEQ ID NO:826), DVS (SEQ ID NO:827), and SSYTSSSPVV (SEQ ID NO:828) respectively;
j) a heavy chain with three CDRs comprising the amino acid sequences GFTFSHYD (SEQ ID NO:781), IGYDGTNL (SEQ ID NO:782), and ARAANYYDSSGYGRADAF (SEQ ID NO:783) respectively, and/or a light chain with three CDRs comprising the amino acid sequences TGSIAGNY (SEQ ID NO:829), DDN (SEQ ID NO:830), and QSYDSGNRGV (SEQ ID NO:831) respectively;
k) a heavy chain with three CDRs comprising the amino acid sequences GGTFSTYG (SEQ ID NO:784), IIPSLGIP (SEQ ID NO:785), and ARENIDLATNDF (SEQ ID NO:786) respectively, and/or a light chain with three CDRs comprising the amino acid sequences SRDIGAYGY (SEQ ID NO:832), EVR (SEQ ID NO:833), and SSYTSSSTLDVV (SEQ ID NO:834) respectively;
l) a heavy chain with three CDRs comprising the amino acid sequences GGTFSSSG (SEQ ID NO:787), IIPMLGTP (SEQ ID NO:788), and ARDGGNYDY (SEQ ID NO:789) respectively, and/or a light chain with three CDRs comprising the amino acid sequences SSNIGRNA (SEQ ID NO:835), SNN (SEQ ID NO:836), and SAWDTSLSTWV (SEQ ID NO:837) respectively;
m) a heavy chain with three CDRs comprising the amino acid sequences GFTFSSYW (SEQ ID NO:790), IKQDGSEK (SEQ ID NO:791), and ARGFYYYGAFDI (SEQ ID NO:792) respectively, and/or a light chain with three CDRs comprising the amino acid sequences SGSIASNY (SEQ ID NO:838), EDN (SEQ ID NO:839), and QSYDSSNHWV (SEQ ID NO:840) respectively;
n) a heavy chain with three CDRs comprising the amino acid sequences GFTFDDYA (SEQ ID NO:793), IDWNSGVI (SEQ ID NO:794), and AKDAYSYGFLGAFDI (SEQ ID NO:795) respectively, and/or a light chain with three CDRs comprising the amino acid sequences NIRTKG (SEQ ID NO:841), YAS (SEQ ID NO:842), and QVWDSSSDLVV (SEQ ID NO:843) respectively;
o) a heavy chain with three CDRs comprising the amino acid sequences GYSFTGSH (SEQ ID NO:796), INPDSGVI (SEQ ID NO:797), and ARDKAIGYVWALDY (SEQ ID NO:798) respectively, and/or a light chain with three CDRs comprising the amino acid sequences SSDVGTYNR (SEQ ID NO:844), EVS (SEQ ID NO:845), and SSYTRTFTYV (SEQ ID NO:846) respectively; or
p) a heavy chain with three CDRs comprising the amino acid sequences GASISNSF (SEQ ID NO:799), TSYSGNS (SEQ ID NO:800), and ARREWIKGHFDY (SEQ ID NO:801) respectively, and/or a light chain with three CDRs comprising the amino acid sequences GGSIASNY (SEQ ID NO:847), EDN (SEQ ID NO:848), and QSYDSSNPVV (SEQ ID NO:849) respectively.

32. The monoclonal antibody of claim 1, wherein the monoclonal antibody comprises:
a) a V_{H} amino acid sequence having SEQ ID NO: 722, and a V_{L} amino acid sequence having SEQ ID NO: 723;
b) a V_{H} amino acid sequence having SEQ ID NO: 724, and a V_{L} amino acid sequence having SEQ ID NO: 725;
c) a V_{H} amino acid sequence having SEQ ID NO: 726, and a V_{L} amino acid sequence having SEQ ID NO: 727;
d) a V_{H} amino acid sequence having SEQ ID NO: 728, and a V_{L} amino acid sequence having SEQ ID NO: 729;
e) a V_{H} amino acid sequence having SEQ ID NO: 730, and a V_{L} amino acid sequence having SEQ ID NO: 731;
f) a V_{H} amino acid sequence having SEQ ID NO: 732, and a V_{L} amino acid sequence having SEQ ID NO: 733;
g) a V_{H} amino acid sequence having SEQ ID NO: 734, and a V_{L} amino acid sequence having SEQ ID NO: 735;
h) a V_{H} amino acid sequence having SEQ ID NO: 736, and a V_{L} amino acid sequence having SEQ ID NO: 737;
i) a V_{H} amino acid sequence having SEQ ID NO: 738, and a V_{L} amino acid sequence having SEQ ID NO: 739;
j) a V_{H} amino acid sequence having SEQ ID NO: 740, and a V_{L} amino acid sequence having SEQ ID NO: 741;
k) a V_{H} amino acid sequence having SEQ ID NO: 742, and a V_{L} amino acid sequence having SEQ ID NO: 743;
l) a V_{H} amino acid sequence having SEQ ID NO: 744, and a V_{L} amino acid sequence having SEQ ID NO: 745;
m) a V_{H} amino acid sequence having SEQ ID NO: 746, and a V_{L} amino acid sequence having SEQ ID NO: 747;
n) a V_{H} amino acid sequence having SEQ ID NO: 748, and a V_{L} amino acid sequence having SEQ ID NO: 749;
o) a V_{H} amino acid sequence having SEQ ID NO: 750, and a V_{L} amino acid sequence having SEQ ID NO: 751; or
p) a V_{H} amino acid sequence having SEQ ID NO: 752, and a V_{L} amino acid sequence having SEQ ID NO: 753.

33. A method of preventing a disease or disorder caused by Severe Acute Respiratory Syndrome coronavirus (SARS-CoV2), the method comprising administering to a subject at risk of suffering from the disease or disorder, a therapeutically effective amount of the monoclonal antibody of claim 1 or the scFv antibody of claim 15.

34. The method of claim 33, wherein the method further comprises administering an anti-viral drug, a viral entry inhibitor, a viral attachment inhibitor, or a combination thereof.

35. The method of claim 33, wherein the method comprises administering two or more antibodies specific to SARS-CoV2.

36. The method of claim 33, wherein the antibody is administered prior to or after exposure to SARS-CoV2.

37. The method of claim 33, wherein the antibody is administered at a dose sufficient to neutralize the SARS-CoV2.

38. A method of delaying the onset of one or more symptoms of a SARS-CoV2 infection, the method comprising administering to a subject at risk of suffering from the infection, a therapeutically effective amount of the monoclonal antibody of claim 1 or the scFv antibody of claim 15.

39. A composition comprising the monoclonal antibody of claim 1 or the scFv antibody of claim 15 and a carrier.

40. A method of detecting the presence of SARS-CoV2 in a sample, the method comprising:
a) contacting the sample with the monoclonal antibody of claim 1 or the scFv antibody of claim 15; and
b) detecting the presence or absence of an antibody-antigen complex, thereby detecting the presence of SARS-CoV2 in a sample.

41. The method of claim 40, wherein the detecting occurs in vivo.

42. The method of claim 40, wherein the sample is obtained from blood, hair, cheek scraping, saliva, biopsy, or semen.

43. The monoclonal antibody of claim 1, wherein the monoclonal antibody blocks the binding of SARS-CoV2 spike protein to angiotensin converting enzyme 2 (ACE2) cell surface receptor.

44. The monoclonal antibody of claim 1, wherein the monoclonal antibody comprises a heavy chain and/or light chain listed in any one of Table 65 to Table 126.

45. The monoclonal antibody of claim 1, wherein the monoclonal antibody comprises a heavy chain with three CDRs comprising any one of the amino acid sequences described in Table 63, and/or a light chain with three CDRs comprising any one of the amino acid sequences described in Table 63.

46. The antibody of claim 15, wherein the antibody comprises a heavy chain and/or light chain listed in any one of Table 65 to Table 126.

47. The antibody of claim 15, wherein the antibody comprises a heavy chain with three CDRs comprising any one of the amino acid sequences described in Table 63, and/or a light chain with three CDRs comprising any one of the amino acid sequences described in Table 63.
